(19) United States
(12) Patent Application Publication
(10) Pub. No.: US 2024/0300972 A1
(43) Pub. Date: Sep. 12, 2024

(54) THIOPHENE-BASED LIPIDS

(71) Applicant: Oregon State University, Corvallis, OR (US)

(72) Inventors: Gaurav Sahay, Corvallis, OR (US); Mohit Gupta, Corvallis, OR (US); Jeonghwan Kim, Corvallis, OR (US); Yulia V. Eygeris, Corvallis, OR (US); Milan Gautam, Corvallis, OR (US); Jonas Renner, Corvallis, OR (US)

(73) Assignee: Oregon State University, Corvallis, OR (US)

(21) Appl. No.: 18/617,043

(22) Filed: Mar. 26, 2024

Related U.S. Application Data
(63) Continuation of application No. PCT/US22/44835, filed on Sep. 27, 2022.
(60) Provisional application No. 63/248,638, filed on Sep. 27, 2021.
(60) Provisional application No. 63/336,800, filed on Apr. 29, 2022.

(51) Int. Cl. C07D 495/04; A61K 9/51; A61K 31/381; A61K 31/4365; A61K 45/06; C07D 333/26

(52) U.S. Cl. CPC C07D 495/04; A61K 9/5123; A61K 9/5146; A61K 31/381; A61K 31/4365; A61K 45/06; C07D 333/26

(57) ABSTRACT

Disclosed herein are compounds according to Formula A

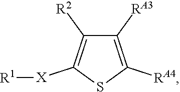

wherein the compound comprises at least one C₆₋₃₀aliphatic moiety. Also disclosed are compositions comprising the compounds that may be useful for delivering agents such as therapeutic and/or prophylactic agents, for example, nucleic acids such as, but not limited to, DNA or RNA, small molecules, proteins, polypeptides or peptides. In some embodiments, the composition is a lipid nanoparticle. Also disclosed herein are lipid nanoparticles comprising the compounds and methods for making and using the nanoparticles.

### RELATED APPLICATIONS

This application is a continuation of PCT/US2022/044835, filed Sep. 27, 2022, which claims the benefit under 35 U.S.C. § 119(e) of U.S. provisional application No. 63/248,638, filed Sep. 27, 2021, and U.S. provisional application No. 63/336,800, filed Apr. 29, 2022, the entire contents of each of which are incorporated herein by reference.

### ACKNOWLEDGMENT OF GOVERNMENT SUPPORT

This invention was made with government support under Grant Nos. R21 EY031066 and R01 HL146736 awarded by the National Institutes of Health. The government has certain rights in the invention.

### FIELD

Disclosed herein are novel thiophene-based compounds, compositions comprising the compounds, and methods for making and using the compounds and compositions.

### BACKGROUND

With the U.S. FDA approval of mRNA vaccines for COVID-19, there has been an unprecedented global interest to develop nucleic acid-based therapeutics. Vectors play an instrumental role in the delivery of gene of interest. Viral, polymer, and cationic lipid-based vector suffers from lack of efficacy and/or toxicity issues. Ionizable lipids are currently the choice for vectors for gene delivery. However, not many advancements have been made with ionizable lipids due to the lack of structural diversity. The current ionizable lipids (ILs) are also, for the major part, difficult to synthesize, are symmetrical in structure, and are non-modular in nature.

### SUMMARY

Herein we report a novel class of thiophene based ionizable/cationic lipids with unprecedented structural diversity and modularity. The thiophene based lipids are easy to synthesize and can deliver a gene of interest.

Disclosed herein is compounds according to Formula A:

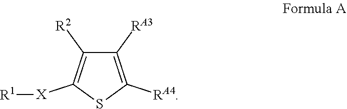

With respect to Formula A, X is a bond, —C(O)O—, —OC(O)—, —NH—, —N(R¹)—, —C(O)N(R¹)—, —N(R¹)C(O)—, —C(O)NH—, —NHC(O)—, —C≡C—, —CH═CH—, —O—, —S—, —NHC(O)NH—, —OC(O)O—, —OC(O)NH—, —NHC(O)O—, —O(CH₂)ₘ— or —(CH₂)ₘO— where m is an integer from 0 to 6, such as from 1 to 6, and in some embodiments, m is 1; R¹ is H, halogen, CN, or aliphatic, and may be aliphatic, such as C₆₋₃₀ aliphatic or C₆₋₁₀ aliphatic; and R² is H, halogen, aromatic, or aliphatic, such as H or C₆₋₃₀ aliphatic. In some embodiments, R¹ and R² are both C₆₋₃₀ aliphatic, and are different. In some embodiments, R¹ and R² are both C₆₋₃₀ aliphatic, and are the same. Alternatively, X is a bond and R¹ and R², together with the atoms to which there are attached, form a 4 to 8-membered nitrogen-containing or non-nitrogen containing non-aromatic heterocyclyl. In further alternative embodiments, X is a bond and R¹ is H, —CN or halogen, such as X is a bond and R¹ is halogen, or X is a bond and R¹ is CN.

R^{A3} is halogen, —R³, —C(O)N(R⁶)(R⁷), —C(O)R³, —CN, —(CH₂)ᵣN(R⁶)(R⁷), —N(R⁶)(R⁷), —S(O)R^{d}, —S(O)R³, —CH₂SR^{d}, —CH₂R³, —S(O)N(R⁶)(R⁷), or —X¹(CH₂)ᵣN(R^{a})₂, and R³ is aliphatic, —O-aliphatic or —N(R⁶)(R⁷). R^{A4} is halogen, boronic acid, —N(R⁴)(R⁵), —NHC(O)N(R⁴)(R⁵), —N═CHR^{d}, —NHC(S)N(R⁴)(R⁵), —NHC(O)(CH₂)_{z}—N(R⁴)(R⁵), —NHSO₂R^{d}, —NHS(O)R^{d}, —NHC(O)C(O)NH(CH₂)_{z}—N(R⁴)(R⁵), or —OR⁴, where z is from 1 to 6. R^{d} is aliphatic, and R⁴ is aliphatic or —C(O)aliphatic, R⁵ is H, aliphatic, or —C(O)aliphatic, R⁶ is H or aliphatic, and R⁷ is aliphatic or —C(O)aliphatic, or R⁶ and R⁷, together with the atom to which there are attached, form a 4- to 7-membered nitrogen-containing heterocyclyl. In certain embodiments, R⁶ is H or aliphatic; and R⁷ is aliphatic or C(O)aliphatic.

Also, with respect to Formula A, the compound comprises at least one C₆₋₃₀ aliphatic moiety.

In some embodiments, the compound has a Formula:

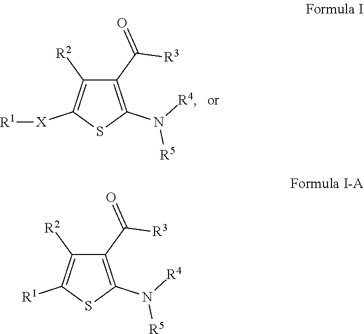

where R¹, R², R³, R⁴ and R⁵ are as defined herein, and X if present is a bond, —NH—, —N(R¹)—, or —OC(O)—.

In some embodiments, e.g., of Formulas A, I and I-A, R¹ is C₁₋₆alkyl and may be substituted with —N(R^{a})₂, where each R^{a} independently is H or C₁₋₆alkyl, or both R^{a}s together with the nitrogen to which they are attached form an optionally substituted 5- or 6-membered heterocyclyl. R¹ may be —(CH₂)₁₋₄N(R^{a})₂, such as —CH₂NH₂.

The compound may have a structure according to the following formula:

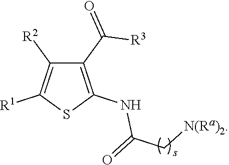

In some embodiments, each of R¹ and R² independently is C₆₋₃₀aliphatic; each R^{a} independently is H or C₁₋₆alkyl, or both R^{a}s together with the nitrogen to which they are attached form an optionally substituted 5- or 6-membered heterocyclyl; and s is from 1 to 8, such as 1, 2, 3, or 4, or 2 or 3. In certain embodiments, s is an integer from 1, 2, 3 or 4. In some embodiments, R³ is —O—C₁₋₈alkyl.

Alternatively, the compound may have a structure according to Formula II:

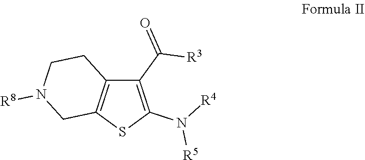

where R⁸ is H or R^{e}; and R^{c} is -aliphatic, -aliphatic-amino, —C(O)-aliphatic, or —C(O)-aliphatic-amino or any nitrogen protecting groups like Boc, Cbz, acetyl, trityl, benzylidenamine amongst others. R^{c} may be —C₆₋₃₀aliphatic, —C₁₋₆aliphatic-amino, —C(O)—C₆₋₃₀aliphatic, or —C(O)—C₁₋₆aliphatic-amino, such as —(CH₂)₁₋₄N(R^{a})₂, where each R^{a} independently is H or C₁₋₆alkyl, or both R^{a}s together with the nitrogen to which they are attached form an optionally substituted 5- or 6-membered heterocyclyl. In some embodiments, R⁸ is —C(O)—C₆₋₃₀aliphatic.

Additionally, or alternatively, the compound may have a formula:

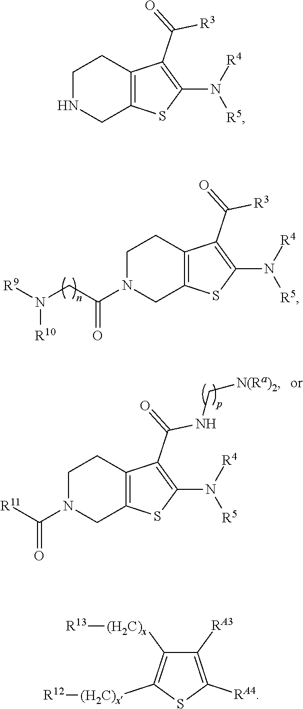

With respect to these formulas, each of R⁹ and R¹⁰ independently is H or C₁₋₆alkyl; or R⁹ and R¹⁰ together with the nitrogen to which they are attached, form an optionally substituted 5- or 6-membered non-aromatic heterocyclyl moiety. n is from 1 to 8. R¹¹ is C₆₋₃₀aliphatic. Each R^{a} independently is H or C₁₋₆alkyl, or both R^{a}s together with the nitrogen to which they are attached form an optionally substituted 5- or 6-membered heterocyclyl. p is from 1 to 8. In certain embodiments, R⁹ is unsubstituted C₁₋₆ alkyl. In certain embodiments, R¹⁰ is unsubstituted C₁₋₆ alkyl. In certain embodiments, p is 1, 2, 3, or 4. Each of R¹² and R¹³ independently is —C(O)O—R^{f}, —OC(O)—R^{f}, —C(O)NH—R^{f}, or —NH(C(O)—R^{f}. Each R^{f} independently is a linear or branched C₆₋₃₀aliphatic. And each of x and x′ independently is from 1 to 8.

Also, disclosed herein is a composition comprising one or more compounds disclosed herein. In some embodiments, the composition is a nanoparticle. The composition, such as a nanoparticle, may further comprise one or more agents, such as therapeutic and/or prophylactic agents. In some embodiments, each agent is selected from a nucleic acid, such as a single stranded DNA, single stranded RNA, double-stranded DNA, RNA-RNA hybrid, DNA-RNA hybrid, shortmer, antagomir, antisense, ribozyme, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), or a combination thereof. Additionally or alternatively, the agent(s) may be selected from a chemotherapeutic drug, small molecule drug, protein, polypeptide, antibody, peptide or a combination thereof.

The disclosed compound may be present in the composition, such as a nanoparticle, an amount of from 0.1 mol % to 100 mol %. And/or the composition may further comprise a phospholipid, a structural lipid, a polymer-conjugated lipid, or a combination thereof. In certain embodiments, the phospholipid is a glycerophospholipid. In certain embodiments, the phospholipid is a phosphatidic acid, phosphatidylethanolamine, phosphatidylcholine, phosphatidylserine, or phosphoinositide. In certain embodiments, the phospholipid is a phosphatidylinositol, phosphatidylinositol phosphate, phosphatidylinositol bisphosphate, or phosphatidylinositol trisphosphate. In certain embodiments, the phospholipid is a sphingolipid. In certain embodiments, the phospholipid is a ceramide phosphorylcholine, ceramide phosphorylethanolamine, or ceramide phosphoryllipid. The structural lipid may be a sterol, for example, cholesterol, beta-sitosterol, fucosterol, campesterol, stigmastanol, or a combination thereof, and/or the polymer-conjugated lipid may be selected from PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. In certain embodiments, the sterol is a phytosterol. In certain embodiments, the sterol is a zoosterol. In certain embodiments, the sterol is a cholesterol. In certain embodiments, the sterol is a campesterol, sitosterol (e.g., beta sitosterol), stigmasterol, or ergosterol. In certain embodiments, the sterol is a hopanoid, hydroxysteroid, or steroid. For example, a PEG lipid may be PEG-c-DOMG, PEG-DMG, PEG-DLPE, PEG-DMPE, PEG-DPPC, a PEG-DSPE lipid, or a combination thereof. The polymer-conjugated lipid may comprise a PEG moiety having a molecular weight of from 1000 daltons to 20,000 daltons.

In some embodiments, the composition, such as a nanoparticle, comprises from 0 to 30 mol % of the phospholipid, from 0 to 60 mol % of the structural lipid, and from 0 to 10 mol % of the polymer-conjugated lipid, with a proviso that at least one of the phospholipid, the structural lipid, and the polymer-conjugated lipid is present in the composition. In some embodiments, the molar ratio of the nucleic acid to the compound is from 2:1 to 30:1. And in some embodiments, a wt/wt ratio of total lipid component to nucleic acid is from 5:1 to 60:1.

In certain embodiments, the polymer-conjugated lipid is (distearoyl-phosphatidyl-ethanolamine)-PEG-C(O)OH or (distearoyl-phosphatidyl-ethanolamine)-PEG-carboxy-NHS. In certain embodiments, the composition further comprises a sterol, carboxy-PEG, 1,2-distearoyl-sn-glycero-3-phosphocholine or a negatively charged phospholipid, and a PEG-lipid. In certain embodiments, the concentration of the compound is 50%±its 30%;
the concentration of the sterol is 38.5%±its 30%;
the concentration of the carboxy-PEG is between 0 and 0.36%, e.g., between 0.15% and 0.35%, inclusive;
the concentration of 1,2-distearoyl-sn-glycero-3-phosphocholine or the negatively charged phospholipid is 10%±its 30%; and/or
the concentration of the PEG-lipid is 1.2%±its 30%;
provided that the combined concentrations of the compound, sterol, carboxy-PEG, 1,2-distearoyl-sn-glycero-3-phosphocholine or negatively charged phospholipid, and PEG-lipid is 100%.

In certain embodiments, the concentration of the compound is 50%±its 20%;
the concentration of the sterol is 38.5%±its 20%;
the concentration of the carboxy-PEG is between 0 and 0.36%, e.g., between 0.15% and 0.35%, inclusive;
the concentration of 1,2-distearoyl-sn-glycero-3-phosphocholine or the negatively charged phospholipid is 10%±its 20%; and/or
the concentration of the PEG-lipid is 1.2%±its 20%;
provided that the combined concentrations of the compound, sterol, carboxy-PEG, 1,2-distearoyl-sn-glycero-3-phosphocholine or negatively charged phospholipid, and PEG-lipid is 100%.

In certain embodiments, the concentration of the compound is 50%±its 10%;
the concentration of the sterol is 38.5%±its 10%;
the concentration of the carboxy-PEG is between 0 and 0.36%, e.g., between 0.15% and 0.35%, inclusive;
the concentration of 1,2-distearoyl-sn-glycero-3-phosphocholine or the negatively charged phospholipid is 10%±its 10%; and/or
the concentration of the PEG-lipid is 1.2%±its 10%;
provided that the combined concentrations of the compound, sterol, carboxy-PEG, 1,2-distearoyl-sn-glycero-3-phosphocholine or negatively charged phospholipid, and PEG-lipid is 100%. In certain embodiments, the sterol is cholesterol or beta-sitosterol.

A method for making the compounds of Formula II-a also is disclosed herein, the method comprises:
when R⁵ is H: reacting a compound of the formula:

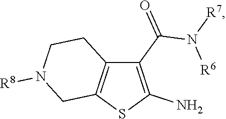

with a compound of the formula: R⁴-(leaving group) or R⁴—OH under suitable conditions to provide the compound; or
when R⁵ is aliphatic or —C(O)aliphatic: reacting a compound of the formula:

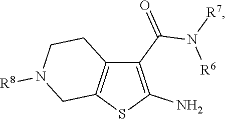

with a compound of the formula: R⁴-(leaving group) or R⁴—OH, and a compound of the formula: R⁵-(leaving group) or R⁵—OH, in the same step or separate steps, under suitable conditions to provide the compound.

A method for making the composition also is disclosed herein. In certain embodiments, the method comprises:
providing a first solution comprising a compound of the present disclosure;
providing a second solution comprising an agent; and
mixing the first and second solutions to form a mixture comprising the nanoparticle.
   The method may comprise providing a first solution comprising a compound or more compounds as disclosed herein, providing a second solution comprising an agent to be delivered, for example, a nucleic acid and/or therapeutic agent, and mixing the first and second solutions to form a mixture comprising the composition. The method may also comprise providing a phospholipid, a structural lipid, a polymer-conjugated lipid, or a combination thereof, and where mixing the first and second solutions further comprises mixing the phospholipid, the structural lipid, and/or the polymer-conjugated lipid to form the mixture. The phospholipid, the structural lipid, the polymer-conjugated lipid, or a combination thereof, may be provided in the first solution. In some embodiments, the composition comprises a nanoparticle. In some embodiments, the composition is a nanoparticle. In some embodiments, the composition comprises a microparticle. In some embodiments, the composition comprises a liposome. In some embodiments, the composition comprises a micelle.

In another aspect, the present disclosure provides a method for making the nanoparticle, the method comprises:
providing a first solution comprising a compound of the present disclosure;
providing a third solution; and
mixing the first and third solutions to form a mixture comprising the nanoparticle.
   In certain embodiments, the third solution is an aqueous buffer solution. In certain embodiments, the method further comprises:
providing a fourth solution comprising an agent; and
mixing the mixture comprising the nanoparticle with the fourth solution to form a composition.

In certain embodiments, the agent is a nucleic acid.

Further disclosed herein are embodiments of a method for using the composition, such as a nanoparticle composition. The method may comprise administering an effective amount of the composition to a subject, such as by a suitable administration route, for example, by an intravenous, intramuscular, or intradermal route. Administering to the subject may comprise administering to lung, liver and/or spleen tissue, and in some embodiments, administering to the subject comprises administering to lung tissue.

In another aspect, the present disclosure provides a method of delivering a pharmaceutical agent to a subject in need thereof comprising administering to or implanting in the subject in need thereof an effective amount of:
a compound or pharmaceutical composition described herein; and
a pharmaceutical agent.

In another aspect, the present disclosure provides a method of treating a disease comprising administering to or implanting in a subject in need thereof an effective amount of:
a compound or pharmaceutical composition described herein; and
a therapeutic agent.

In another aspect, the present disclosure provides a method of preventing a disease comprising administering to or implanting in a subject in need thereof an effective amount of:
a compound or pharmaceutical composition described herein; and
a prophylactic agent.

In another aspect, the present disclosure provides a method of diagnosing a disease comprising administering to or implanting in a subject in need thereof an effective amount of:
a compound or pharmaceutical composition described herein; and
a diagnostic agent.

Also disclosed herein is a use a compound disclosed herein, or a composition comprising the same, in the preparation of a medicament for administration to a subject.

Also disclosed herein is a kit comprising:
a compound or composition described herein; and
instructions for using the compound or composition.

The foregoing and other objects, features, and advantages of the invention will become more apparent from the following detailed description, which proceeds with reference to the accompanying figures.

### BRIEF DESCRIPTION OF THE DRAWINGS

FIG. 1 is a graph of size and polydispersity index (PdI or PDI) versus exemplary lipid nanoparticles (LNPs), illustrating the size and polydispersity of nanoparticles comprising certain exemplary disclosed compounds.

FIG. 2 is a graph of RNA encapsulation versus exemplary lipid nanoparticles (LNPs) illustrating the encapsulation efficiency of nanoparticles made by exemplary lipids disclosed herein.

FIG. 3 is a graph of in vitro transfection efficacy of the LNPs versus mRNA dose, illustrating the dose response for nanoparticles made from disclosed lipids 19da, 26a, and 26b.

FIG. 4 is a graph of in vitro transfection efficacy of the LNPs made from lipid versus mRNA dose, illustrating the dose response for nanoparticles made from disclosed lipids 29d, 34a, and 34b.

FIG. 5 is a graph of in vitro transfection efficacy of the LNPs made from lipid versus mRNA dose, illustrating the dose response for nanoparticles made from disclosed lipid 35a.

FIG. 6 is a graph of total flux versus lipids, illustrating the luminescent signals detected by in vivo imaging after in vivo administration of the nanoparticles, and indicating the respective levels of successful transfection.

FIG. 7 is a digital image illustrating the location of the luminescent signal in animals administered with nanoparticles containing lipid 34a.

FIG. 8 is a digital image illustrating that the luminescence shown in FIG. 7 was located in the liver and spleen.

FIG. 9 is a digital image illustrating that the luminescent signal in animals administered with nanoparticles containing lipid 29d was located in the lungs and spleen.

FIG. 10 is a digital image illustrating the location of the luminescent signals in animals administered with nanoparticles containing lipids 19da, 26b, and 26a.

FIG. 11 is a graph of total flux versus lipid, illustrating the luminescent signal resulting from intramuscular administration of nanoparticles containing lipids 19da and 26a.

FIG. 12 is a digital image illustrating the locations of the luminescence following intramuscular injection of nanoparticles containing lipids 19da and 26a.

FIG. 13 is a graph of total flux versus time, illustrating the luminescent signal resulting from subretinal injection of nanoparticles containing lipids 19da, 26a and 26b.

FIG. 14 is a digital image illustrating the locations of the luminescence following administration of nanoparticles containing lipids 19da and 26a.

FIGS. 15A to 15F show the design and characterization of Cre mRNA encapsulated LNP variants. FIG. 15A is a schematic of the structural organization of different LNPs containing mRNAs. FIG. 15B is a graph of lipid composition of LNPs and DSPE-PEG functionalized LNPs (R-LNPs). FIG. 15C is a graph of hydrodynamic size and PDI of Cre mRNA cargo loaded LNPs determined by DLS. FIG. 15D is a graph of Cre mRNA encapsulation of different functional PEG containing LNPs. FIG. 15E is a graph of changes in surface charge of Cre mRNA cargo loaded LNP systems with changing the functional PEG determined by ZetaView instrument. Statistical comparison was done against the DMG-PEG2k added LNP system. All Data are presented as mean±SD. An ordinary one-way ANOVA, with Tukey's correction for multiple comparisons test was used for comparisons. *p<0.05, **p<0.01, ***p<0.001. FIG. 15F shows digital cryo-TEM images of functional PEG-modified LNPs.

FIGS. 16A to 16E show in-vivo injection, tdTomato expression in the fundus and retina of Ai9 mice after subretinal administration of lipid nanoparticles. FIG. 16A is a schematic illustration of lipid nanoparticles administration in Cre mouse and tdTomato expression in the eye. FIG. 16B shows representative bright field and tdTomato expression in fundus images taken at 7-day post-injection and confocal images of immunohistochemistry showing tdTomato expression in the RPE, the PR, and the Müller glia for all groups following sub-retinal injection. FIG. 16C shows low magnification confocal images of LNPx treated group. Arrowhead indicates tdTomato expressions. FIG. 16D is a graph of flow cytometry quantitation of cellular uptake of Cy5-tagged lipid nanoparticles by 661 w cone cells for 24 hours. Fold changes values are relative to untreated control. FIG. 16E is a graph of fundus quantification of tdTomato intensity, represented as a fold change compared to PBS treated group. All Data are presented as mean±SD. An ordinary one-way ANOVA, with Tukey's correction for multiple comparisons test, was used for comparisons. n.s.—not significant, *p<0.05, **p<0.01, ***p<0.001 ****p<0.0001. n=3-6. RPE: RPE: retinal pigment epithelium, PR: photoreceptor, ONL: outer nuclear layer, OPL: outer plexiform layer, INL: inner nuclear layer.

FIGS. 17A to 171 show recoverin co-localization with tdTomato in the mouse retina. Images of retinal sections from Ai9 mice co-stained with rabbit anti-recoverin antibody detected with Alexa-700-conjugated anti-rabbit secondary antibody and tdTomato expression post-7-day administration via subretinal route. Nuclei were counter-stained with DAPI. FIGS. 17A to 17F show representative confocal images of immunohistochemistry showing expression of tdTomato in the RPE, PR, ONL, and INL for all groups. Arrowhead indicates tdTomato expressions. Quantification of tdTomato expression in six different sections of the retina using Fiji ImageJ software in the (FIG. 17G) photoreceptor layer (inset: the magnified confocal image of LNPx treated group showing photoreceptor and photoreceptor nuclei), (FIG. 17H) outer nuclear layer (inset: A higher magnification of confocal images of eLNPx treated group, showing photoreceptor nuclei), and (FIG. 17I) inner nuclear layer. All Data are presented as mean±SD. An ordinary one-way ANOVA, with Tukey's correction for multiple comparisons test, was used for comparisons. n.s.—not significant, *p<0.05, n=6. RPE: retinal pigment epithelium, PR: photoreceptor, ONL: outer nuclear layer, INL: inner nuclear layer.

FIGS. 18 and 19 show intracellular uptake of Cy-5 tagged LNP and R-LNPs. FIG. 18 shows laser confocal microscope images of 661 W cone cells transfected with Cy-5 tagged LNPs and R-LNPs at 24 hours treatment time. All the cells were treated with Cy5-tagged lipid nanoparticles dose equivalent to 100 ng/well of mRNA cargo (Scale bar: 25 um). FIG. 19 is a graph of relative fluorescence fold changes of Cy-5 tagged LNPs obtained by quantitative estimation of confocal images against untreated control. All Data are presented as mean±SD. An ordinary one-way ANOVA, with Tukey's correction for multiple comparisons test was used for comparisons. **p<0.01, ***p<0.001.

FIGS. 20A and 20B show efficient cell uptake of Cy-5 tagged LNP and R-LNPs. FIG. 20A shows a representative FACS plot for uptake of 661 W cone cells transfected with various LNPs including PBS control at 24 hours after single dose 100 ng/well treatment of Cy-5 tagged LNP and R-LNPs in-vitro (representative of n=3 samples). FIG. 20B is a representative FACS histogram of Cy-5 tagged LNPs and R-LNPs obtained form same uptake study.

FIGS. 21A to 21D show in-vitro gene transfection efficiency of the LNP and R-LNPs. Graph depicts the cell viability and transfection efficiencies (normalized to PBS treated group) of the LNPs and R-LNPs in the 661 w cone cells at (FIGS. 21A to 21B) 24 hours and (FIGS. 21C to 21D) 48 hours treatment intervals. All Data are presented as mean±SD. An ordinary one-way ANOVA, with Tukey's correction for multiple comparisons test was used for comparisons. *p<0.05, **p<0.01, ***p<0.001 ****p<0.0001. (n=5).

FIGS. 22A to 22D show in-vitro gene transfection efficiency of the LNP and R-LNPs. Graph depicts the cell viability and transfection efficiencies (normalized to PBS treated group) of the LNPs and R-LNPs in the HeLa cells at (FIGS. 22A to 22B) 24 hours and (FIGS. 22C to 22D) 48 hours treatment intervals. All Data are presented as mean±SD. An ordinary one-way ANOVA, with Tukey's correction for multiple comparisons test was used for comparisons. *p<0.05, **p<0.01, ****p<0.0001. (n=5).

FIGS. 23A to 23D show in-vitro gene transfection efficiency of the LNP and R-LNPs. Graph depicts the cell viability and transfection efficiencies (normalized to PBS treated group) of the LNPs and R-LNPs in the HEK293T cells at (FIGS. 23A to 23B) 24 hours and (FIGS. 23C to 23D) 48 hours treatment intervals. All Data are presented as mean±SD. An ordinary one-way ANOVA, with Tukey's correction for multiple comparisons test was used for comparisons. ****p<0.0001. (n=5).

FIG. 24 shows representative bright field and tdTomato expression in fundus images taken at 7-day post-injection and confocal images of immunohistochemistry showing tdTomato expression in the RPE, the PR and the Müller glia for all groups following sub-retinal injection in Ai9 mice. Arrowhead indicates tdTomato expressions n=3-6. RPE: RPE: retinal pigment epithelium, PR: photoreceptor, ONL: outer nuclear layer, INL: inner nuclear layer.

FIGS. 25A to 25C show in-vivo tdTomato expression in the retina of Ai9 mice with subretinal administration of LNPs and R-LNPs at 7-day post-injection. Confocal microscopic images of retina cryo-sections showing tdTomato expression in the RPE, the PR and the Müller glia following sub-retinal injection of (FIG. 25A) PBS, (FIG. 25B) LNP and (FIG. 25C) LNPa. Arrowhead indicates tdTomato expressions.

FIGS. 26A to 26C show in-vivo tdTomato expression in the retina of Ai9 mice with subretinal administration of LNPs and R-LNPs at 7-day post-injection. Confocal microscopic images of retina cryo-sections showing tdTomato expression in the RPE, the PR and the Müller glia following sub-retinal injection of (FIG. 26A) LNPx, (FIG. 26B) eLNPx and (FIG. 26C) LNPz. Arrowhead indicates tdTomato expressions.

FIGS. 27A to 27B show Ai9 mouse retinal sections co-stained with rabbit anti-recoverin antibody detected with Alexa-700-conjugated anti-rabbit secondary antibody and tdTomato expression post 7-day administration via subretinal route. Nuclei were counter stained with DAPI. Confocal images of immunohistochemistry showing expression of tdTomato in the RPE for (FIG. 27A) PBS, (FIG. 27B) LNP administered group. The arrowhead indicates tdTomato expression in RPE. (PR: photoreceptor, ONL: outer nuclear layer, INL: inner nuclear layer).

FIGS. 28A to 28B show Ai9 mouse retinal sections co-stained with rabbit anti-recoverin antibody detected with Alexa-700-conjugated anti-rabbit secondary antibody and tdTomato expression post 7-day administration via subretinal route. Nuclei were counter stained with DAPI. Confocal images of immunohistochemistry showing expression of tdTomato in the RPE, PR, ONL and INL for (FIG. 28A) LNPs, and (FIG. 28B) LNPx administered group. Arrowhead indicates tdTomato expressions. (PR: photoreceptor, ONL: outer nuclear layer, INL: inner nuclear layer).

FIGS. 29A to 29B show Ai9 mouse retinal sections co-stained with rabbit anti-recoverin antibody detected with Alexa-700-conjugated anti-rabbit secondary antibody and tdTomato expression post 7-day administration via subretinal route. Nuclei were counter stained with DAPI. Confocal images of immunohistochemistry showing expression of tdTomato in the RPE, PR, ONL and INL for (FIG. 29A) eLNPx, and (FIG. 29B) LNPz administered group. Arrowhead indicates tdTomato expressions. (PR: photoreceptor, ONL: outer nuclear layer, INL: inner nuclear layer).

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B” may mean “including A, B, or A and B,” without excluding additional elements (e.g., C). All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited. Unless otherwise indicated, ranges also include the end points. A range in the format “AA %±its BB %,” wherein AA and BB are a number between 0 and 100, exclusive, refers to the range between AA %(1−BB %) and AA %×(1+BB %), inclusive.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include implicit hydrogens such that each carbon conforms to a valence of four. For example, in the structure on the left-hand side of the schematic below there are nine hydrogen atoms implied. The nine hydrogen atoms are depicted in the right-hand structure.

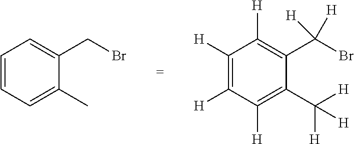

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

The compounds according to the present disclosure may be in a free base form, i.e., not in a salt form, or the compounds may be in a salt form, such as a pharmaceutically acceptable salt as defined herein. In certain embodiments, the compound is not in a pharmaceutically acceptable salt form. A person of ordinary skill in the art will understand that in such a salt form the compound may have either a negative or a positive charge, and/or may include a counter ion, such as an organic and/or inorganic counter ion as known to a person of ordinary skill in the art, and/or as described herein. In some embodiments, the compound may be in a zwitterion form having both a positive and negative charge. The overall change of such a compound may be zero and/or it may not have a separate counter ion.

Additionally, or alternatively, the disclosed compound may be in a non-solvated form or it may be solvated, as defined herein.

The compounds of the present disclosure may also contain unnatural proportions of atomic isotopes at one or more of atoms that constitute such compounds. For example, the compounds may be radiolabeled with radioactive isotopes, such as, for example, tritium (3H), iodine-125 (125I) or carbon-14 (14C), and/or contain an unnatural proportion of non-radioactive isotopes, such as deuterium, or carbon-13 (C¹³). All isotopic variations of the compounds of the present disclosure, whether radioactive or not, are encompassed within the scope of the present disclosure.

In some embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl may be C₂H₅ or C₂H₅ where from 1 to 5 hydrogens are replaced by deuterium, such as in C₂DₓH₅₋ₓ.

In some embodiments, one or more carbon atoms present in a compound may be replaced with a carbon-13 or carbon-14. Thus, a recitation of alkyl includes carbon-13 and/or carbon-14 alkyl, where from one to the maximum number of carbon atoms present may be replaced by either carbon-13 or carbon-14. For example, ethyl may be C₂H₅ or C₂H₅ where one or both carbons are replaced by carbon-13, carbon-14, or a mixture thereof.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms.

Additionally, or alternatively, the disclosed compound may be in a non-co-crystal form or it may be in a co-crystal form, as defined herein.

Additionally, or alternatively, the disclosed compound may be in a polymorph form (e.g., being amorphous or crystalline), as defined herein.

Any group or moiety may be optionally substitute, i.e., may be substituted or unsubstituted, unless otherwise specified, for example, as “unsubstituted” or “substituted.” In particular embodiments, the group or moiety may or may not be expressly defined as substituted, but is still contemplated to be optionally substituted. For example, “aliphatic” may be substituted or unsubstituted aliphatic, and “heterocyclyl” may be substituted or unsubstituted heterocyclyl. A substituted group or moiety has at least one, and may be two or more, hydrogen atoms of the specified group or moiety independently replaced with the same or different substituents groups. In certain embodiments, suitable substituents include halogen, ═O, —C(O)OR, —SH, —SSR, —NHC(O)NR₂, P(═O)(OR)₂, —OR, —OC(O)NR₂, —NHC(O)OR, ═NNHC(O)R, —SO₂NR₂, ketal, thioketal, —OC(O)OR, —NHCSNR₂, ═NOR, —C(OR)₃, hydroxyl, amine, carbonyl (C═O), aldehyde, or aliphatic, such as alkyl, alkenyl, alkynyl, and straight chain, cyclic and branched versions thereof, where each R independently is H, aliphatic, aromatic, or 2 R's together with the atoms to which they are attached, form a 3- to 8-membered heterocyclyl comprising from 1, 2, or 3 heteroatoms selected from N, O and S. Typically, each R independently is H, C₁₋₈aliphatic, C₆aryl, 3- to 8-membered heteroaryl comprising from 1, 2, or 3 heteroatoms selected from N, O and S, or 2 R's together with the atoms to which they are attached, form a 3- to 8-membered heterocyclyl. In some embodiments, each R independently is H or C₁₋₆alkyl. In certain embodiments, a straight chain is unbranched unsubstituted aliphatic (e.g., alkyl, alkenyl, alkynyl). In certain embodiments, a straight chain is unbranched aliphatic (e.g., alkyl, alkenyl, alkynyl) optionally substituted only with one or more substituents that are not aliphatic.

Carbon atoms of the groups and moieties described herein are substituted or unsubstituted, as valency permits. Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)₃⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents are joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

Nitrogen atoms of the groups and moieties described herein are substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, 5-14 membered heteroaryl, or oxo, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

Oxygen atoms of the groups and moieties described herein are substituted or unsubstituted as valency permits. In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R′)₃, —P(R^{cc})₂, —P(R^{c})₃⁺X⁻, —P(OR^{cc})₂, —P(OR^{c})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

Sulfur atoms of the groups and moieties described herein are substituted or unsubstituted as valency permits. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group. In certain embodiments, at least one sulfur atom substituent is oxo.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{c})₃⁺X⁻, —P(OR^{cc})₂, —P(OR^{cc})₃⁺X⁻, —P(═O)(R′)₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. In some embodiments, an anionic counterion is monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

In some embodiments, a group that is substituted has at least one substituent up to the number of substituents possible for a particular moiety, such as 1 substituent, 2 substituents, 3 substituents, or 4 substituents.

Additionally, in embodiments where a group or moiety is substituted with a substituted substituent, the nesting of such substituted substituents is limited to three, thereby preventing the formation of polymers. Thus, in a group or moiety comprising a first group that is a substituent on a second group that is itself a substituent on a third group, which is attached to the parent structure, the first (outermost) group can only be substituted with unsubstituted substituents. For example, in a group comprising -(aryl-1)-(aryl-2)-(aryl-3), aryl-3 can only be substituted with substituents that are not themselves substituted.

And the term “substituted” refers to all subsequent modifiers in a term, for example in the term “substituted “-alkylaryl,” substitution may occur on the “alkyl” portion, the “aryl” portion or both portions of the alkylaryl group.

“Aliphatic” refers to a substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and all stereo and position isomers as well. In some embodiments, an aliphatic group is linear or branched but includes a cyclic moiety within a linear or branched. Unless expressly stated otherwise, an aliphatic group contains from one to thirty carbon atoms (C₁₋₃₀) or more; for example, from one to twenty five (C₁₋₂₅), from one to twenty (C₁₋₂₀), one to fifteen (C₁₋₁₅), from one to ten (C₁₋₁₀), from one to six (C₁₋₆), or from one to four carbon atoms (C₁₋₄) for a saturated acyclic aliphatic group or moiety, from two to thirty carbon atoms (C₂₋₃₀); for example, from two to twenty five (C₂₋₂₅), from two to twenty (C₂₋₂₀), two to fifteen (C₂₋₁₅), from two to ten (C₂₋₁₀), from two to six (C₂₋₆), or from two to four carbon atoms (C₂₋₄) for an unsaturated acyclic aliphatic group or moiety, or from three to fifteen (C₃₋₁₅) from three to ten (C₃₋₁₀), from three to six (C₃₋₆), or from three to four (C₃₋₄) carbon atoms for a cycloaliphatic group or moiety. In some embodiments, a saturated aliphatic moiety, such as an alkyl or cycloalkyl moiety, may have from 1-8 carbon atoms, such as from 1-6, or 1-4 carbon atoms; an unsaturated aliphatic moiety may have from 2-8 carbon atoms, such as 2-6 or 2-4 carbon atoms; or a cyclic aliphatic moiety may have from 3 to 8 carbon atoms, such as from 3-6 carbon atoms. But certain other aliphatic moieties may have from 6-30 carbon atoms, such as from 6-25 carbon atoms, from 6-22 carbon atoms, from 6-20 carbon atoms, from 6-18 carbon atoms, from 8-20 carbon atoms, from 8-18 carbon atoms, from 10-20 carbon atoms, from 12-20 carbon atoms, from 14-20 carbon atoms, from 14-18 carbon atoms, or from 16-18 carbon atoms.

An aliphatic group may be substituted or unsubstituted, i.e., optionally substituted, unless expressly referred to as an “unsubstituted aliphatic” or a “substituted aliphatic.” An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Substituents on an aliphatic group or moiety may be any substituents understood by a person of ordinary skill in the art to be compatible with the synthesis and/or use of the ionizable lipid compounds.

“Alkyl” refers to a saturated aliphatic hydrocarbyl group having, unless otherwise specified, from 1 to 30 (C₁₋₃₀) or more carbon atoms, such as from 1 to 10 (C₁₋₁₀) carbon atoms, from 1 to 6 (C₁₋₆) carbon atoms, or from 6 to 30 carbon atoms such as from 6-25 carbon atoms, from 6-22 carbon atoms, from 6-20 carbon atoms, from 6-18 carbon atoms, from 8-20 carbon atoms, from 8-18 carbon atoms, from 10-20 carbon atoms, from 12-20 carbon atoms, from 14-20 carbon atoms, from 14-18 carbon atoms, or from 16-18 carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), neopentyl (—CH₂C(CH₃)₃), hexyl (C₆H₁₃), heptyl (C₇H₁₅), octyl (C₈H₁₇), decyl (C₁₀H₂₁), dodecyl (C₁₂H₂₅), tetradecyl (C₁₄H₂₉), hexadecyl (C₁₆H₃₃), heptadecyl (C₁₇H₃₅), octadecyl (C₁₈H₃₇), or eicosanyl (C₂₀H₄₁).

“Alkenyl” refers to an unsaturated aliphatic hydrocarbyl group having, unless otherwise specified, at least 1 double bond and from 2 to 30 (C₂₋₃₀) or more carbon atoms, such as from 2 to 10 (C₂₋₁₀) carbon atoms, from 2 to 6 (C₂₋₆) carbon atoms, or from 6 to carbon atoms such as from 6-25 carbon atoms, from 6-22 carbon atoms, from 6-20 carbon atoms, from 6-18 carbon atoms, from 8-20 carbon atoms, from 8-18 carbon atoms, from 10-20 carbon atoms, from 12-20 carbon atoms, from 14-20 carbon atoms, from 14-18 carbon atoms, or from 16-18 carbon atoms. An alkenyl moiety may be substituted or unsubstituted. This term includes, by way of example, linear and branched hydrocarbyl groups, such as vinyl, allyl, but-3-en-1-yl. Also included within this term are the cis and trans isomers or mixtures of these isomers, unless otherwise specified.

“Alkynyl” refers to an unsaturated aliphatic hydrocarbyl group having, unless otherwise specified, at least 1 triple bond and from 2 to 30 (C₂₋₃₀) or more carbon atoms, such as from 2 to 10 (C₂₋₁₀) carbon atoms, from 2 to 6 (C₂₋₆) carbon atoms, or from 6 to 30 carbon atoms such as from 6-25 carbon atoms, from 6-22 carbon atoms, from 6-20 carbon atoms, from 6-18 carbon atoms, from 8-20 carbon atoms, from 8-18 carbon atoms, from 10-20 carbon atoms, from 12-20 carbon atoms, from 14-20 carbon atoms, from 14-18 carbon atoms, or from 16-18 carbon atoms. An alkynyl moiety may be substituted or unsubstituted. This term includes, by way of example, linear and branched hydrocarbyl groups, such as ethynyl, 1-propynyl and 2-propynyl.

“Amino” refers to a —N(R)R′ moiety where R and R′ are independently H, aliphatic, such as alkyl, alkenyl or alkynyl, or cyclic versions thereof, or R and R′ together with the nitrogen to which they are attached form a 5- to 7-membered heterocyclic ring, optionally containing one, two or three further heteroatoms selected from O, N, and S, and/or optionally substituted with one, two or three aliphatic groups, such as alkyl groups.

A “basic nitrogen atom” refers to the nitrogen atom to which R and R′ are attached in an amino moiety, wherein the nitrogen atom is not attached to any one of —C(O)—, —S(O)—, —S(O)₂—, —P(O), —P(O)₂, and

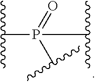 [SMILES: *P(*)(*)=O]

In certain embodiments, R and R′ together with the nitrogen atom to which they are attached do not form a heteroaryl ring.

“Aromatic” refers to a cyclic, conjugated group or moiety of, unless specified otherwise, from 5 to 15 ring atoms having a single ring (e.g., phenyl, pyridinyl, or pyrazolyl) or multiple condensed (e.g., fused) rings in which at least one ring is aromatic (e.g., naphthyl, indolyl, or pyrazolopyridinyl), that is at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

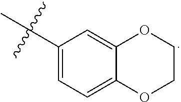 [SMILES: *c1ccc2c(c1)OCCO2]

However, in certain examples, context or express disclosure may indicate that the point of attachment is through a non-aromatic portion of the condensed ring system. For example,

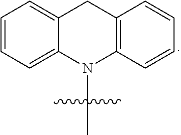 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Unless otherwise stated, an aromatic group may be substituted or unsubstituted.

“Aryl” refers to an aromatic carbocyclic group of, unless specified otherwise, from 6 to 15 carbon atoms having a single ring (e.g., phenyl) or multiple condensed rings in which at least one ring is aromatic (e.g., 1,2,3,4-tetrahydroquinoline, benzodioxole, and the like). If any aromatic ring portion contains a heteroatom, the group is heteroaryl and not aryl. Aryl groups may be, for example, monocyclic, bicyclic, tricyclic or tetracyclic. Unless otherwise stated, an aryl group may be substituted or unsubstituted.

“Boronic acid” refers to a —B(OR)₂ moiety where each R independently is H, aliphatic or aromatic, or both R's together with the atoms to which they are attached, form a heterocyclyl moiety.

“Halogen” or “halo” refers to F, Cl, Br or I.

“Heteroaryl” refers to an aromatic group or moiety of, unless specified otherwise, from 5 to 15 ring atoms comprising at least one carbon atom and at least one heteroatom, such as N, S, or O. A heteroaryl group or moiety may comprise a single ring (e.g., pyridinyl, pyrimidinyl or pyrazolyl) or multiple condensed rings (e.g., indolyl, benzopyrazolyl, or pyrazolopyridinyl). Heteroaryl groups or moiety may be, for example, monocyclic, bicyclic, tricyclic or tetracyclic. Unless otherwise stated, a heteroaryl group or moiety may be substituted or unsubstituted.

“Heterocyclyl” refer to both aromatic and non-aromatic ring systems, and more specifically refer to a stable three- to fifteen-membered ring moiety comprising carbon atoms and at least one, such as from one to five heteroatoms selected from O, N, and S. The heterocyclyl moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems; and the nitrogen, phosphorus, carbon, silicon or sulfur atoms in the heterocyclyl moiety can be optionally oxidized to various oxidation states. For convenience, nitrogens, particularly but not exclusively, those defined as annular aromatic nitrogens, are meant to include their corresponding N-oxide form, although not explicitly defined as such in a particular example. Unless specified as “aromatic” or “non-aromatic,” heterocycle includes heteroaryl moieties, and heterocycloaliphatic moieties, which are non-aromatic heterocyclyl rings that are partially or fully saturated. Exemplary heterocyclyl groups include, but are not limited to, 4-piperidonyl, pyrrolidinyl, pyrazolidinyl, imidazolinyl, piperazinyl, piperidinyl, morpholinyl, homopiperazinyl, homopiperidinyl, aziridine, azetidine, oxirane, thiirane, lactam, lactone, thietane, tetrahydopyran, azocane, oxepane, quinuclidine, azaadmantane, indoline, dihydroquinoline, thiomorpholine, thiane, tetrahydrofuran, tetrahydropyran, 1,3-dioxalane, thienyl, furanyl, pyrrolyl, imidazolyl, pyrazolyl, triazolyl, pyridinyl, pyrimidinyl, pyrazinyl, oxazolyl, thiazolyl, oxadiazolyl, thiadiazolyl, quinolyl, and indolyl.

A heterocyclyl group may be substituted or unsubstituted, i.e., optionally substituted, unless expressly referred to as an “unsubstituted heterocyclyl” or a “substituted heterocyclyl.”

A “leaving group” is given its ordinary meaning in the art of synthetic organic chemistry and refers to an atom or a group capable of being displaced by a nucleophile. Examples of suitable leaving groups include halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate.

“Lipid” refers to an organic compound that is readily soluble in nonpolar solvents such as hydrocarbons, but typically is sparingly or non-soluble in water, and may be poorly soluble in other polar solvents. Ionizable lipids are lipids that can be ionized, for example, with pH-dependent ionization. The lipid may be anionic and/or cationic, for example, it may form an anion and/or a cation depending on pH. In some embodiments, an ionizable lipid may be positive at low pH, and may be substantially neutral at physiological or neutral pH.

“MG-lipid” refers to a compound of the present disclosure, e.g., a compound of Formula A.

“Nanoparticle” as used herein refers to a composition, such as a pharmaceutical formulation, having a particle size (for example, a diameter) of from 1 to 1000 nanometers, such as from 1 to 500 nanometers or from 1 to 100 nanometers, and incorporating one or more lipid compounds disclosed herein. In certain embodiments, the average (e.g., mean) dimension (e.g., diameter or length) of a nanoparticle is between 1 and 3, between 3 and 10, between 10 and 30, between 30 and 100, between 100 and 300, or between 300 and 1,000, nm, inclusive. In certain embodiments, the average dimension is determined with dynamic light scattering. Nanoparticle compositions include, but are not limited to, lipid nanoparticles (LNPs), liposomes (e.g., lipid vesicles), and lipoplexes.

“Microparticle” refers to a particle having an average (e.g., mean) dimension (e.g., diameter or length) of between 1 and 3, between 3 and 10, between 10 and 30, between 30 and 100, between 100 and 300, or between 300 and 1,000, μm, inclusive. In certain embodiments, the average dimension is determined with dynamic light scattering.

“Lipid nanoparticle” (LNP) refers to a nanoparticle comprising one or more lipid compounds. Typically, the lipid compound(s) will be a major component of the nanoparticle. LNPs may be substantially spherical in shape. Disclosed LNPs may be positively charged in low pH and substantially neutral at physiological pH. Alternatively, the LNP may be uncharged, even if the lipids themselves are charged. In some embodiments, the ionizable lipid is contained in the core and its charge may be shielded by other lipid components.

“Nucleic acid” refers to a polynucleotide molecule. The polynucleotide may be a naturally occurring polynucleotide or a synthetic polynucleotide. A nucleic acid may be a DNA, RNA or mixture of DNA and RNA nucleotides. Typically, the nucleic acid contains from 20 to 10,000 nucleotides or more, such as from 100, 200, 300, 400, 500, 600, 700, 800, 900, 1000, 2000, 3000, 4000, or 5000 nucleotides to 10,000 nucleotides.

Exemplary nucleic acids include, but are not limited to, single stranded DNA, single stranded RNA, double stranded DNA, RNA-RNA hybrid, DNA-RNA hybrid, shortmer, antagomir, antisense, ribozyme, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), or a combination thereof.

“Peptide” refers to a compound comprising amino acid residues connected by peptide bonds. Typically a peptide compound has from 2 to about 50 amino acid residues.

“Polypeptide” refers to a compound comprising amino acid residues connected by peptide bonds. When the amino acids are alpha-amino acids, either the L-optical isomer or the D-optical isomer can be used. In some embodiments, a polypeptide has from about 50 amino acid residues to 2000 or more amino acid residues.

“Protein” refers to a molecule or complex comprising one or more polypeptides having secondary, tertiary and/or quaternary structure. The secondary, tertiary and/or quaternary structure of a protein typically is stabilized using non-covalent bonds, such as ionic bonds, hydrogen bonds, hydrophobic interactions, and/or van der Walls interactions. Additionally, or alternatively, a protein may include disulfide bonds, such as between the thiol groups of cysteine residues. In certain embodiments, a peptide, polypeptide, or protein comprises (e.g., consists essentially of) between 3 and 10, between 10 and 30, between 30 and 100, between 100 and 300, between 300 and 1,000, between 1,000 and 3,000, or between 3,000 and 10,000, inclusive, amino acids. In certain embodiments, the amino acids are natural amino acids. In certain embodiments, the amino acids are unnatural amino acids. In certain embodiments, the amino acids are a combination of natural amino acids and unnatural amino acids (e.g., unnatural alpha-amino acids).

“Small Molecule” refers to an organic molecule having a molecular weight of about 2000 Daltons or less. In some embodiments, the term “small molecule” refers to a compound that is not a polypeptide, protein, or nucleic acid molecule. A small molecule may be a small molecule therapeutic and/or prophylactic, such as an antibiotic, anti-inflammatory, anticancer, antiviral, immunosuppressant, analgesic, antifungal, antiparasitic, anticonvulsants, antidepressant, anti-anxiety, anti-psychotic, and the like. In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 g/mol, not more than 900 g/mol, not more than 800 g/mol, not more than 700 g/mol, not more than 600 g/mol, not more than 500 g/mol, not more than 400 g/mol, or not more than 300 g/mol. In certain embodiments, the molecular weight of a small molecule is between 200 and 300 g/mol, between 300 and 400 g/mol, between 400 and 600 g/mol, between 600 and 800 g/mol, between 800 and 1,000 g/mol, between 1,000 and 1,300 g/mol, between 1,300 and 1,600 g/mol, or between 1,600 and 2,000 g/mol,

“Pharmaceutically acceptable excipient” refers to a substantially physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, as a carrier, flavoring, thickener, diluent, buffer, preservative, or surface active agent and/or to modify properties of a pharmaceutical composition. Examples of excipients include, but are not limited, to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

“Pharmaceutically acceptable salt” refers to pharmaceutically acceptable salts of a compound that are derived from a variety of organic and inorganic counter ions as will be known to a person of ordinary skill in the art and typically include, by way of example only, sodium, potassium, calcium, magnesium, ammonium, tetraalkylammonium, hydrochloride, hydrobromide, tartrate, mesylate, acetate, maleate, oxalate, and the like. In particular, the disclosed compounds may form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, benzene sulfonic acid, isethionic acid, methanesulfonic acid, ethanesulfonic acid, p-toluenesulfonic acid, salicylic acid, and the like. (See, for example, S. M. Berge, et al., “Pharmaceutical Salts,” J. Pharm. Sci., 1977; 66:1-19 which is incorporated herein by reference.)

“Solvate” refers to a complex formed by combination of solvent molecules with molecules or ions of the solute. The solvate may be a hydrate.

The term “hydrate” refers to a compound, or a salt thereof, that is associated with water. Typically, the number of the water molecules contained in a hydrate of a compound, or a salt thereof, is in a definite ratio to the number of the compound molecules in the hydrate. Therefore, in some embodiments, a hydrate of a compound, or a salt thereof, is represented, for example, by the general formula R·x H₂O, wherein R is the compound, or a salt thereof, and x is a number greater than 0. A given compound, or a salt thereof, may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. In some embodiments, co-crystals are useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. In some embodiments, various polymorphs of a compound (or a salt, hydrate, or solvate thereof) are prepared by crystallization under different conditions.

“Subject” refers to mammals and other animals, particularly humans. Thus disclosed methods are applicable to both human therapy and veterinary applications. In certain embodiments, the human is male or female, of any age group, e.g., pediatric subject (e.g., infant, child, or adolescent) or adult subject (e.g., young adult, middle-aged adult, or senior adult). In certain embodiments, the mammal is a primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal is a male or female at any stage of development. In some embodiments, the non-human animal is a transgenic animal or genetically engineered animal.

The term “tissue” refers to any biological tissue of a subject (including a group of cells, a body part, or an organ) or a part thereof, including blood and/or lymph vessels. In some embodiments, “tissue” is the object to which a compound, particle, and/or composition of the disclosure is delivered. In some embodiments, a tissue is an abnormal or unhealthy tissue, which may need to be treated. A tissue may also be a normal or healthy tissue that is under a higher than normal risk of becoming abnormal or unhealthy, which may need to be prevented.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound or pharmaceutical composition described herein in a single dose. In certain embodiments, an effective amount is the combined amounts of a compound or pharmaceutical composition described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher's disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The cancer may be a solid tumor. The cancer may be a hematological malignancy. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, dermatitis (e.g., stasis dermatitis, allergic contact dermatitis, atopic dermatitis, irritant contact dermatitis, neurodermatitis perioral dermatitis, seborrheic dermatitis), hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, inflammatory rosacea. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “metabolic disease” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

The term “psychiatric disorder” refers to a condition or disorder relating to the functioning of the brain and the cognitive processes or behavior. Psychiatric disorders may be further classified based on the type of neurological disturbance affecting the mental faculties. Psychiatric disorders are expressed primarily in abnormalities of thought, feeling, emotion, and/or behavior producing either distress or impairment of function (for example, impairment of mental function such with dementia or senility). The term “psychiatric disorder” is, accordingly, sometimes used interchangeably with the term “mental disorder” or the term “mental illness”. A psychiatric disorder is often characterized by a psychological or behavioral pattern that occurs in an individual and is thought to cause distress or disability that is not expected as part of normal development or culture. Definitions, assessments, and classifications of mental disorders can vary, but guideline criteria listed in the International Classification of Diseases and Related Health Problems (ICD, published by the World Health Organization, WHO), or the Diagnostic and Statistical Manual of Mental Disorders (DSM, published by the American Psychiatric Association, APA) and other manuals are widely accepted by mental health professionals. Individuals may be evaluated for various psychiatric disorders using criteria set forth in these and other publications accepted by medical practitioners in the field and the manifestation and severity of a psychiatric disorder may be determined in an individual using these publications.

### II. Compounds

Disclosed herein, in one aspect, are compounds that may be suitable for use in delivery applications for one or more therapeutic agents, such as nucleic acid agents such as mRNA, protein, polypeptide, small molecule therapeutics and the like. In some embodiments, the compound is a lipid, and may be an ionizable lipid. And/or in some embodiments, the therapeutic agent is mRNA.

In some embodiments, delivery applications comprising the disclosed compounds demonstrate improved in vitro transfection, compared to delivery applications comprising existing lipids, such as MC3. And the disclosed compounds exhibit low toxicity in a cytotoxicity assay. And in some embodiments, compositions comprising the disclosed compounds may selectively target organs, such as the liver, lungs and/or spleen, or they may be systemic.

In some embodiments, the ionizable lipids are compounds have a structure according to Formula A:

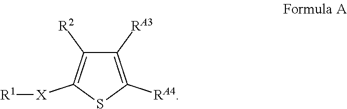

With respect to Formula A:
X is a bond, —C(O)O—, —OC(O)—, —NH—, —N(R¹)—, —C(O)N(R¹)—, —N(R¹)C(O)—, —C(O)NH—, —NHC(O)—, —C≡C—, —CH═CH—, —O—, —S—, —NHC(O)NH—, —OC(O)O—, —OC(O)NH—, —NHC(O)O—, —O(CH₂)ₘ— or —(CH₂)ₘO— where m is an integer from 0 to 6, such as from 1 to 6, and in some embodiments, m is 1.

R¹ is H, halogen, —CN, or aliphatic. In some embodiment, R¹ is aliphatic, such as C₁₋₃₀aliphatic. In some embodiments, R¹ is C₆₋₃₀aliphatic, such as C₆₋₂₅aliphatic, C₆₋₂₀aliphatic, C₆₋₁₈aliphatic, C₆₋₁₆aliphatic, C₆₋₁₄aliphatic, or C₆₋₁₀aliphatic, and may be saturated (i.e., alkyl), or unsaturated (i.e., alkenyl or alkynyl, typically alkenyl, and may have 1, 2 or 3 unsaturated bonds), and/or may include cyclic moieties, such as C₅₋₇cycloalkyl moieties. In certain embodiments, R¹ is C₇₋₈alkyl, such as C₇alkyl. In other embodiments, R¹ is C₁₋₆aliphatic, such as C₁₋₆alkyl, or C₁₋₄alkyl, and may be methyl, ethyl, propyl or isopropyl.

In any embodiments, R¹ may be a straight chain, branched and/or cyclic aliphatic, such as a straight chain or branched alkyl or alkenyl, or cyclic version thereof, or R¹ may be a straight chain or branched moiety that also comprises a cyclic moiety.

And in any embodiments, R¹ may be unsubstituted or substituted, such as with amino, C(O)OR^{b}, or OC(O)R^{b}, where R^{b} is C₁₋₂₀aliphatic, such as C₁₋₁₈aliphatic, C₅₋₁₈aliphatic, or C₁₀₋₁₈ aliphatic, and may be saturated (i.e., alkyl), or unsaturated (i.e., alkenyl or alkynyl, typically alkenyl, and may have 1, 2 or 3 unsaturated bonds), and/or may include cyclic moieties, such as C₅₋₇cycloalkyl moieties. In some embodiments, R^{b} is C₁₂₋₁₆alkyl, and maybe C₁₅alkyl.

In certain embodiments, R¹ is C₁₋₆ alkyl substituted with amino, such as —(CH₂)₁₋₄N(R^{a})₂ where each R^{a} independently is H; C₁₋₆alkyl, such as C₁₄alkyl, and may be methyl, ethyl, propyl, or isopropyl; C₃₋₆cycloalkyl, such as cyclopropyl, cyclobutyl, cyclopentyl, or cyclohexyl; or both R^{a}s together with the nitrogen to which they are attached form a 5- or 6-membered heterocyclyl, optionally interrupted by one or more heteroatoms, such as O, N or S, and optionally substituted with C₁₋₆alkyl. In certain embodiments, R^{a} is methyl. In some embodiments, the alkyl of R^{a} is unsubstituted. In some embodiments, the alkyl of R^{a} is substituted at least or only with halogen (e.g., F). In some embodiments, the alkyl of R^{a} comprises no unsaturated CC bonds. In some embodiments, the alkyl of R^{a} comprises only one unsaturated CC bond. In some embodiments, the alkyl of R^{a} comprises only two unsaturated CC bonds. In some embodiments, the alkyl of R^{a} is unbranched. In some embodiments, the alkyl of R^{a} comprises only one branch. In some embodiments, the alkyl of R^{a} comprises only two branches.

In some embodiments, X is a bond and R¹ is H, —CN or halogen.

In particular embodiments, X is a bond, and R¹ is —CH₂NH₂.

R² is H, halogen, such as F, Cl, Br or I, aliphatic, or aromatic, such as aryl or heteroaryl. In some embodiments, R² is H or aliphatic, such as C₁₋₃₀aliphatic. In some embodiments, R² is H, but in other embodiments, R² is aliphatic, such as C₁₋₃₀aliphatic, and may be C₆₋₃₀aliphatic, C₆₋₂₅aliphatic, C₆₋₂₀aliphatic, C₆₋₁₈aliphatic, C₆₋₁₆aliphatic, C₆₋₁₄aliphatic, or C₆₋₁₀aliphatic. R² may be saturated (i.e., alkyl), or unsaturated (i.e., alkenyl or alkynyl, typically alkenyl and may have 1, 2 or 3 unsaturated bonds), and/or may include cyclic moieties, such as C₅₋₇cycloalkyl moieties. In some embodiments, if R² is aliphatic, such as C₆₋₃₀aliphatic, then R¹ also is C₆₋₃₀aliphatic.

In other embodiments, R² is C₁₋₆aliphatic, such as C₁₋₆alkyl, and may be methyl, ethyl, propyl or isopropyl, butyl or pentyl.

R² may be substituted with —OC(O)R^{b} or —C(O)OR^{b}, where R^{b} is as defined above with respect to R¹.

In alternative embodiments, X is a bond, and R¹ and R², together with the atoms to which there are attached, form a 4- to 8-membered non-aromatic heterocyclyl, such as a 5- to 7-membered non-aromatic heterocyclyl, and may be a nitrogen-containing or non-nitrogen-containing non-aromatic heterocyclyl. In some embodiments, the non-aromatic heterocyclyl may be a 6-membered nitrogen-containing non-aromatic heterocyclyl, and/or may be optionally substituted. In some embodiments, the nitrogen-containing non-aromatic heterocyclyl may be substituted on a ring nitrogen with R^{c} where R^{c} is -aliphatic, -aliphatic-amino, —C(O)-aliphatic, or —C(O)-aliphatic-amino, such as —C₆₋₃₀aliphatic, —C₁₋₆aliphatic-amino (for example, —C₁₋₆alkyl-amino), —C(O)—C₆₋₃₀aliphatic or —C(O)—C₁₋₆aliphatic-amino (for example, —C(O)—C₁₋₆alkyl-amino). In any embodiments, R^{c} may comprise an aliphatic moiety that is a straight chain, branched and/or may include a cyclic moiety, for example, a C₅₋₇cyclic moiety, and/or may be saturated or may include 1, 2 or 3 double and/or triple bonds. In some embodiments, R^{c} is —C(O)—C₇aliphatic (e.g., caproyl), —C(O)—C₁₁aliphatic (e.g., lauroyl), —C(O)—C₁₅aliphatic (e.g., palmitoyl), —C(O)—C₁₇aliphatic (e.g., stearoyl, oleoyl, or linoleoyl), C₈aliphatic (e.g., capryl), C₁₂aliphatic (e.g., lauryl), C₁₆aliphatic (e.g., (palmityl), or C₁₈aliphatic (e.g., stearyl, oleyl, or linoleyl). In other embodiments, R^{c} is —(CH₂)₁₋₄N(R^{a})₂ where R^{a} is as defined herein. R^{c} may be optionally substituted as defined herein. In some embodiments where R^{c} is -aliphatic or —C(O)-aliphatic, R^{c} may be unsubstituted or R^{c} may be substituted with —C(O)OR^{d} or —OC(O)R^{d}, where R^{d} is as defined herein.

R^{A3} is halogen, R³, —C(O)N(R⁶)(R⁷), —C(O)R³, —CN, —(CH₂)ᵣN(R⁶)(R⁷), —N(R⁶)(R⁷), —S(O)R^{d}, —S(O)R³, —CH₂SR^{d}, —CH₂R³, —S(O)N(R⁶)(R⁷), or —X¹(CH₂)ᵣN(R^{a})₂, where X¹ is O, S or NH, r is from 1 to 8, and each R^{a} independently is H; C₁₋₆alkyl, such as C₁₋₄alkyl, and may be methyl, ethyl, propyl, or isopropyl; C₃₋₆cycloalkyl, such as cyclopropyl, cyclobutyl, cyclopentyl, or cyclohexyl; or both R^{a}s together with the nitrogen to which they are attached form a 5- or 6-membered heterocyclyl, optionally interrupted by one or more heteroatoms, such as O, N or S, and optionally substituted with C₁₋₆alkyl. In some embodiments, R^{A3} is —C(O)R³, —CN, —(CH₂)ᵣN(R⁶)(R⁷), —N(R⁶)(R⁷), —S(O)R^{d}, —S(O)R³, —CH₂SR^{d}, —CH₂R³, —S(O)N(R⁶)(R⁷), or —X¹(CH₂)ᵣN(R^{a})₂. In some embodiments, R^{A3} is C₇₋₁₀ aliphatic, C₁₋₄ aliphatic, C₁₅₋₁₈ aliphatic, C₁₉₋₂₃ aliphatic, or C₂₄₋₃₀ aliphatic. In some embodiments, the aliphatic of R^{A3} is unsubstituted. In some embodiments, the aliphatic of R^{A3} is substituted at least or only with halogen (e.g., F). In some embodiments, R^{A3} is —C(O)(C₇₋₁₀ aliphatic), —C(O)(C₁₁₋₁₄ aliphatic), —C(O)(C₁₅₋₁₈ aliphatic), —C(O)(C₁₉₋₂₃ aliphatic), or —C(O)(C₂₄₋₃₀ aliphatic). In some embodiments, the aliphatic of R^{A3} comprises no unsaturated CC bonds. In some embodiments, the aliphatic of R^{A3} comprises only one unsaturated CC bond. In some embodiments, the aliphatic of R^{A3} comprises only two unsaturated CC bonds. In some embodiments, the aliphatic of R^{A3} is unbranched. In some embodiments, the aliphatic of R^{A3} comprises only one branch. In some embodiments, the aliphatic of R^{A3} comprises only two branches.

R³ is aliphatic, such as C₁₋₃₀aliphatic; —O-aliphatic, such as —O—C₁₋₃₀aliphatic; or —N(R⁶)(R⁷). In some embodiments, R³ is —O—C₁₋₈aliphatic, such as —O—C₁₋₆aliphatic or —O—C₁₋₄aliphatic, and/or may be saturated, such as, —O—C₁₋₆alkyl or —O—C₁₋₄alkyl. In such embodiments, R³ may be a straight chain, branched, or cyclic —O-aliphatic, and in particular embodiments, R³ is —O-methyl, —O-ethyl, —O-propyl, —O-isopropyl or —O-cyclopropyl.

In other embodiments, R³ is —O—C₆₋₃₀aliphatic, such as —O—C₆₋₂₅aliphatic, —O—C₈₋₂₀aliphatic, —O—C₁₂₋₂₀aliphatic, —O—C₁₄₋₂₀aliphatic, —O—C₁₆₋₂₀aliphatic, or —O—C₁₆₋₁₈aliphatic, and may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties. In particular examples of such embodiments, R³ is —O—C₁₆₋₂₀alkyl, such as —O—C₁₆₋₁₈alkyl, or —O—C₁₆alkyl, —O—C₁₇alkyl or —O—C₁₈alkyl, or R³ is —O—C₁₆₋₂₀alkenyl, such as —O—C₁₆₋₁₈alkenyl, or —O—C₁₆alkenyl, —O—C₁₇alkenyl, or —O—C₁₈alkenyl.

Alternatively, R³ is —N(R⁶)(R⁷).

R⁶ is H or aliphatic, such as H or C₁₋₃₀aliphatic, and R⁷ is aliphatic or C(O)aliphatic, such as C₁₋₃₀aliphatic or C(O)—C₁₋₃₀aliphatic, or R⁶ and R⁷, together with the atom to which there are attached, form a 4- to 7-membered nitrogen-containing heterocyclyl. In certain embodiments, R⁶ is H or aliphatic, such as H or C₁₋₃₀aliphatic, and R⁷ is aliphatic or C(O)aliphatic, such as C₁₋₃₀aliphatic or C(O)—C₁₋₃₀aliphatic. In some embodiments, R⁶ is H, but in other embodiments, R⁶ is C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic, and may be saturated (i.e., alkyl), or unsaturated (i.e., alkenyl or alkynyl, typically alkenyl with 1, 2, or 3 double bonds), and/or may be linear or branched and/or include cyclic moieties, such as C₅₋₇cycloalkyl moieties. In some embodiments, R⁶ is C₈aliphatic (e.g., capryl), C₁₂aliphatic (e.g., lauryl), C₁₆aliphatic (e.g., (palmityl), or C₁₈aliphatic (e.g., stearyl, oleyl, or linoleyl). In some embodiments, R⁶ is C₇₋₁₀ aliphatic, C₁₁₋₁₄ aliphatic, C₁₅₋₁₈ aliphatic, C₁₉₋₂₃ aliphatic, or C₂₄₋₃₀ aliphatic. In some embodiments, the aliphatic of R⁶ is unsubstituted. In some embodiments, the aliphatic of R⁶ is substituted at least or only with halogen (e.g., F). In some embodiments, the aliphatic of R⁶ comprises no unsaturated CC bonds. In some embodiments, the aliphatic of R⁶ comprises only one unsaturated CC bond. In some embodiments, the aliphatic of R⁶ comprises only two unsaturated CC bonds. In some embodiments, the aliphatic of R⁶ is unbranched. In some embodiments, the aliphatic of R⁶ comprises only one branch. In some embodiments, the aliphatic of R⁶ comprises only two branches.

R⁷ may be aliphatic or —C(O)aliphatic. In some embodiments, R⁷ is C₁₋₈aliphatic, such as C₁₋₆aliphatic or C₁₄aliphatic, optionally substituted, such as with amino, but in other embodiments, R⁷ is C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic, and may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁷ is C₈aliphatic (e.g., capryl), C₁₂aliphatic (e.g., lauryl), C₁₆aliphatic (e.g., (palmityl), or C₁₈aliphatic (e.g., stearyl, oleyl, or linoleyl). Alternatively, R⁷ is —C(O)aliphatic, and may be —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic, and may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁷ is —C(O)—C₇aliphatic (e.g., caproyl), —C(O)—C₁₁aliphatic (e.g., lauroyl), —C(O)—C₁₅aliphatic (e.g., palmitoyl), or —C(O)—C₁₇aliphatic (e.g., stearoyl, oleoyl, or linoleoyl). In some embodiments, R⁷ is C₇₋₁₀ aliphatic, C₁₁₋₁₄ aliphatic, C₁₅₋₁₈ aliphatic, C₁₉₋₂₃ aliphatic, or C₂₄₋₃₀ aliphatic. In some embodiments, the aliphatic of R⁷ is unsubstituted. In some embodiments, the aliphatic of R⁷ is substituted at least or only with halogen (e.g., F). In some embodiments, R⁷ is —C(O)(C₇₋₁₀ aliphatic), —C(O)(C₁₁₋₁₄ aliphatic), —C(O)(C₁₅₋₁₈ aliphatic), —C(O)(C₁₉₋₂₃ aliphatic), or —C(O)(C₂₄₋₃₀ aliphatic). In some embodiments, the aliphatic of R⁷ comprises no unsaturated CC bonds. In some embodiments, the aliphatic of R⁷ comprises only one unsaturated CC bond. In some embodiments, the aliphatic of R⁷ comprises only two unsaturated CC bonds. In some embodiments, the aliphatic of R⁷ is unbranched. In some embodiments, the aliphatic of R⁷ comprises only one branch. In some embodiments, the aliphatic of R⁷ comprises only two branches.

In any embodiments, R³ may be substituted, such as by amino.

In some embodiments, R³ is —N(R⁶)(R⁷) where R⁶ is as defined above and R⁷ is C₁₋₆aliphatic substituted with amino, such as —(CH₂)₁₋₆N(R^{a})₂ or —(CH₂)₁₋₄N(R^{a})₂, where each R^{a} independently is as defined above with respect to R¹. In certain embodiments, R³ is —NH(CH₂)₂₋₄N(C₁₋₆alkyl)₂, and may be —NHCH₂CH₂CH₂NMe₂.

R^{A4} is halogen, boronic acid, —N(R⁴)(R⁵), NHC(O)N(R⁴)(R⁵), —N═CHR^{d}, —NHC(S)N(R⁴)(R⁵), —NHC(O)(CH₂)_{z}—N(R⁴)(R⁵), —NHSO₂R^{d}, —NHS(O)R^{d}, —NHC(O)C(O)NH(CH₂)_{z}—N(R⁴)(R⁵), or —OR⁴, where z is from 1 to 6. In some embodiments, R^{A4} is —NHC(O)(C₇₋₃₀ aliphatic). In some embodiments, R^{A4} is —NHC(O)(C₇₋₁₀ aliphatic), —NHC(O)(C₁₁₋₁₄ aliphatic), —NHC(O)(C₁₅₋₁₈ aliphatic), —NHC(O)(C₁₉₋₂₃ aliphatic), or —NHC(O)(C₂₄₋₃₀ aliphatic). In some embodiments, R^{A4} is —N[C(O)(C₇₋₃₀ aliphatic)]₂. In some embodiments, R^{A4} is —N[C(O)(C₇₋₁₀ aliphatic)]₂, —N[C(O)(C₁₁₋₁₄ aliphatic)]₂, —N[C(O)(C₁₅₋₁₈ aliphatic)]₂, —N[C(O)(C₁₉₋₂₃ aliphatic)]₂, or —N[C(O)(C₂₄₋₃₀ aliphatic)]₂. In some embodiments, R^{A4} comprises no unsaturated CC bonds. In some embodiments, R^{A4} comprises only one unsaturated CC bond. In some embodiments, R^{A4} comprises only two unsaturated CC bonds.

R^{d} is aliphatic, such as C₁₋₃₀aliphatic. R^{d} may be a straight chain, branched, or cyclic aliphatic. In some embodiments, R^{d} is C₁₋₈aliphatic, such as C₁₋₆aliphatic or C₁₄aliphatic, and/or may be saturated, such as C₁₋₆alkyl or C₁₄alkyl. In particular embodiments, R^{d} is methyl, ethyl, propyl, isopropyl or cyclopropyl. In other embodiments, R^{d} is C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic, and may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties. In some embodiments, at least one R^{d} is C₇₋₁₀ aliphatic, C₁₁₄ aliphatic, C₁₅₋₁₈ aliphatic, C₁₉₋₂₃ aliphatic, or C₂₄₋₃₀ aliphatic. In some embodiments, the aliphatic of R^{d} is unsubstituted. In some embodiments, the aliphatic of R^{d} is substituted at least or only with halogen (e.g., F). In some embodiments, the aliphatic of R^{d} comprises no unsaturated CC bonds. In some embodiments, the aliphatic of R^{d} comprises only one unsaturated CC bond. In some embodiments, the aliphatic of R^{d} comprises only two unsaturated CC bonds. In some embodiments, the aliphatic of R^{d} is unbranched. In some embodiments, the aliphatic of R^{d} comprises only one branch. In some embodiments, the aliphatic of R^{d} comprises only two branches.

R⁴ is aliphatic or —C(O)aliphatic, such as C₁₋₃₀aliphatic or —C(O)C₁₋₃₀aliphatic, optionally substituted, such as with amino, and R⁵ is H, aliphatic or —C(O)aliphatic, optionally substituted, such as with amino, —OC(O)R^{d} or —C(O)OR^{d}.

R⁴ may be C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic; or —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic. R⁴ may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁴ is —C(O)—C₇aliphatic (e.g., caproyl), —C(O)—C₁₁aliphatic (e.g., lauroyl), —C(O)—C₁₅aliphatic (e.g., palmitoyl), or —C(O)—C₁₇aliphatic (e.g., stearoyl, oleoyl, or linoleoyl).

In other embodiments, R⁴ is C₁₋₈aliphatic, such as C₁₋₆alkyl, C₁₋₄alkyl or C₂₄alkyl; or —C(O)C₁₋₈aliphatic, such as —C(O)C₁₋₆alkyl, —C(O)C₁₋₄alkyl or —C(O)C₂₋₄alkyl. In such embodiments, R⁵ is substituted with N(R^{a})₂, where each R^{a} independently is as defined above with respect to R¹. In some embodiments, R⁴ is —C(O)(CH₂)₁₋₃N(Me)₂. In some embodiments, R⁴ is C₇₋₁₀ aliphatic, C₁₁₋₁₄aliphatic, C₁₅₋₁₈ aliphatic, C₁₉₋₂₃ aliphatic, or C₂₄₋₃₀ aliphatic. In some embodiments, R⁴ is —C(O)(C₇₋₁₀ aliphatic), —C(O)(C₁₁₋₁₄ aliphatic), —C(O)(C₁₅₋₁₈ aliphatic), —C(O)(C₁₉₋₂₃ aliphatic), or —C(O)(C₂₄₋₃₀ aliphatic). In some embodiments, the aliphatic of R⁴ is unsubstituted. In some embodiments, the aliphatic of R⁴ is substituted at least or only with halogen (e.g., F). In some embodiments, the aliphatic of R⁴ comprises no unsaturated CC bonds. In some embodiments, the aliphatic of R⁴ comprises only one unsaturated CC bond. In some embodiments, the aliphatic of R⁴ comprises only two unsaturated CC bonds. In some embodiments, the aliphatic of R⁴ is unbranched. In some embodiments, the aliphatic of R⁴ comprises only one branch. In some embodiments, the aliphatic of R⁴ comprises only two branches.

In some embodiments, R⁵ is H, but in other embodiments, R⁵ is C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic; or R⁵ is —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic. R⁵ may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁵ is —C(O)—C₇aliphatic (e.g., caproyl), —C(O)—C₁₁aliphatic (e.g., lauroyl), —C(O)—C₁₅aliphatic (e.g., palmitoyl), or —C(O)—C₁₇aliphatic (e.g., stearoyl, oleoyl, or linoleoyl). In some embodiments, R⁵ is C₇₋₁₀ aliphatic, C₁₋₁₄ aliphatic, C₁₅₋₁₈ aliphatic, C₁₉₋₂₃ aliphatic, or C₂₄₋₃₀ aliphatic. In some embodiments, R⁵ is —C(O)(C₇₋₁₀ aliphatic), —C(O)(C₁₁₋₁₄ aliphatic), —C(O)(C₁₅₋₁₈ aliphatic), —C(O)(C₁₉₋₂₃ aliphatic), or —C(O)(C₂₄₋₃₀ aliphatic). In some embodiments, the aliphatic of R⁵ is unsubstituted. In some embodiments, the aliphatic of R⁵ is substituted at least or only with halogen (e.g., F). In some embodiments, the aliphatic of R⁵ comprises no unsaturated CC bonds. In some embodiments, the aliphatic of R⁵ comprises only one unsaturated CC bond. In some embodiments, the aliphatic of R⁵ comprises only two unsaturated CC bonds. In some embodiments, the aliphatic of R⁵ is unbranched. In some embodiments, the aliphatic of R⁵ comprises only one branch. In some embodiments, the aliphatic of R⁵ comprises only two branches.

In certain disclosed examples, R⁴ is —C(O)C₁₋₈aliphatic substituted with N(R^{a})₂ and R⁵ is H. In other disclosed examples, R⁴ is —C(O)—C₆₋₃₀aliphatic and R⁵ is H or —C(O)—C₆₋₃₀aliphatic.

Also with respect to Formula A, the compound comprises at least one C₆₋₃₀aliphatic moiety. In some embodiments of Formula A one of the following conditions applies:
if R² is H, then at least one of R¹, R^{A3}, or R^{A4} is or comprises C₆₋₃₀aliphatic. In certain embodiments, when R² is H, then R^{A4} comprises C₆₋₃₀aliphatic;
if R² is aliphatic, such as C₆₋₃₀aliphatic, then R¹ also is C₆₋₃₀aliphatic; or
if R¹ and R², together with the atoms to which they are attached, form a heterocyclyl moiety, then at least one of R^{A3} and R^{A4} is or comprises C₆₋₃₀aliphatic, or the heterocyclic moiety formed by R¹ and R² is substituted with a substituent that comprises a C₆₋₃₀aliphatic moiety.

In some embodiments, the R¹—X— moiety is R¹—, R¹—NH—, R¹—N(R¹)—, or R¹—OC(O)—; R^{A3} is C(O)R³; and R^{A4} is N(R⁴)(R⁵).

In some embodiments, the compounds have a structure according to the formula:

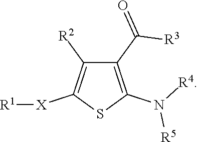

In some embodiments, the compounds have a structure according to Formula I:

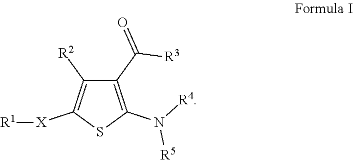

With respect to Formula I, R¹, R², R³, R⁴ and R⁵ are as defined above with respect to Formula A, and X is a bond, —NH—, —N(R¹)—, or —OC(O)—. In certain embodiments, X is a bond.

In certain embodiments, R¹ is aliphatic;
R² is H or aliphatic;
or R¹ and R², together with the atoms to which there are attached, form a 5- to 7-membered nitrogen-containing non-aromatic heterocyclyl;
R³ is aliphatic, —O-aliphatic or N(R⁶)(R⁷);
R⁴ is aliphatic or —C(O)aliphatic;
R⁵ is H, aliphatic or —C(O)aliphatic;
R⁶ is H or aliphatic;
R⁷ is aliphatic or C(O)aliphatic; and
wherein the compound comprises at least one C₆₋₃₀aliphatic moiety.

In some embodiments, R¹ is aliphatic, such as C₁₋₃₀aliphatic. In some embodiments, R¹ is C₆₋₃₀aliphatic, such as C₆₋₂₅aliphatic, C₆₋₂₀aliphatic, C₆₋₁₈aliphatic, C₆₋₁₆aliphatic, C₆₋₁₄aliphatic, or C₆₋₁₀aliphatic, and may be saturated (i.e., alkyl), or unsaturated (i.e., alkenyl or alkynyl, typically alkenyl, and may have 1, 2 or 3 unsaturated bonds), and/or may include cyclic moieties, such as C₅₋₇cycloalkyl moieties. In certain embodiments, R¹ is C₇₋₈alkyl, such as C₇alkyl. In other embodiments, R¹ is C₁₋₆aliphatic, such as C₁₋₆alkyl, or C₁₋₄alkyl, and may be methyl, ethyl, propyl or isopropyl. In some embodiments, R¹ is C₇₋₁₀ aliphatic, C₁₋₁₄ aliphatic, C₁₅₋₁₈ aliphatic, C₁₉₋₂₃ aliphatic, or C₂₄₋₃₀ aliphatic. In some embodiments, the aliphatic of R¹ is unsubstituted. In some embodiments, the aliphatic of R¹ is substituted at least or only with halogen (e.g., F). In some embodiments, the aliphatic of R¹ comprises no unsaturated CC bonds. In some embodiments, the aliphatic of R¹ comprises only one unsaturated CC bond. In some embodiments, the aliphatic of R¹ comprises only two unsaturated CC bonds. In some embodiments, the aliphatic of R¹ is unbranched. In some embodiments, the aliphatic of R¹ comprises only one branch. In some embodiments, the aliphatic of R¹ comprises only two branches. In any embodiments, R¹ may be a straight chain, branched and/or cyclic aliphatic, such as a straight chain or branched alkyl, or alkenyl, or is, or comprises, a cycloalkyl.

And in any embodiments, R¹ may be unsubstituted or substituted, such as with amino, C(O)OR^{b} or OC(O)R^{b}, where R^{b} is C₁₋₂₀aliphatic, such as C₁₋₁₈aliphatic, C₅₋₁₈aliphatic, or C₁₀₋₁₈aliphatic, and may be saturated (i.e., alkyl), or unsaturated (i.e., alkenyl or alkynyl, typically alkenyl, and may have 1, 2 or 3 unsaturated bonds), and/or may include cyclic moieties, such as C₅₋₇ cycloalkyl moieties. In some embodiments, R^{b} is C₁₂₋₁₆alkyl, and maybe C₁₅alkyl. In some embodiments, R^{b} is C₁₋₃ aliphatic, C₄₋₆ aliphatic, C₇₋₉ aliphatic, C₁₀₋₁₂ aliphatic, C₁₃₋₁₆ aliphatic, or C₁₇₋₂₀ aliphatic. In some embodiments, the aliphatic of R^{b} is unsubstituted. In some embodiments, the aliphatic of R^{b} is substituted at least or only with halogen (e.g., F). In some embodiments, the aliphatic of R^{b} comprises no unsaturated CC bonds. In some embodiments, the aliphatic of R^{b} comprises only one unsaturated CC bond. In some embodiments, the aliphatic of R^{b} comprises only two unsaturated CC bonds. In some embodiments, the aliphatic of R^{b} is unbranched. In some embodiments, the aliphatic of R^{b} comprises only one branch. In some embodiments, the aliphatic of R^{b} comprises only two branches.

In certain embodiments, R¹ is C₁₋₆alkyl substituted with amino, such as —(CH₂)₁₋₄N(R^{a})₂ where each R^{a} independently is H; C₁₋₆alkyl, such as C₁₄alkyl, and may be methyl, ethyl, propyl, or isopropyl; C₃₋₆cycloalkyl, such as cyclopropyl, cyclobutyl, cyclopentyl, or cyclohexyl; or both R^{a}s together with the nitrogen to which they are attached form a 5- or 6-membered heterocyclyl, optionally interrupted by one or more heteroatoms, such as O, N or S, and optionally substituted with C₁₋₆alkyl. In certain embodiments, R^{a} is methyl.

In particular embodiments, R¹ is —CH₂NH₂.

R² is H or aliphatic, such as C₁₋₃₀aliphatic. In some embodiments, R² is H, but in other embodiments, R² is aliphatic, such as C₁₋₃₀aliphatic, and may be C₆₋₃₀aliphatic, C₆₋₂₅aliphatic, C₆₋₂₀aliphatic, C₆₋₁₈aliphatic, C₆₋₁₆aliphatic, C₆₋₁₄aliphatic, or C₆₋₁₀aliphatic. R² may be saturated (i.e., alkyl), or unsaturated (i.e., alkenyl or alkynyl, typically alkenyl and may have 1, 2 or 3 unsaturated bonds), and/or may include cyclic moieties, such as C₅₋₇cycloalkyl moieties. In some embodiments, if R² is aliphatic, such as C₆₋₃₀aliphatic, then R¹ also is C₆₋₃₀aliphatic. In some embodiments, R² is C₇₋₁₀ aliphatic, C₁₋₁₄ aliphatic, C₁₅₋₁₈ aliphatic, C₁₉₋₂₃ aliphatic, or C₂₄₋₃₀ aliphatic. In some embodiments, the aliphatic of R² is unsubstituted. In some embodiments, the aliphatic of R² is substituted at least or only with halogen (e.g., F). In some embodiments, the aliphatic of R² comprises no unsaturated CC bonds. In some embodiments, the aliphatic of R² comprises only one unsaturated CC bond. In some embodiments, the aliphatic of R² comprises only two unsaturated CC bonds. In some embodiments, the aliphatic of R² is unbranched. In some embodiments, the aliphatic of R² comprises only one branch. In some embodiments, the aliphatic of R² comprises only two branches.

In other embodiments, R² is C₁₋₆aliphatic, such as C₁₋₆alkyl, and may be methyl, ethyl, propyl or isopropyl, butyl or pentyl.

R² may be substituted with —OC(O)R^{b}, —C(O)OR^{b}, —C(O)N(R^{b})₂, —C(O)NHR^{b}, —OR^{b}, —SSR^{b}, or —NHC(O)R^{b} where each R^{b} independently is as defined above with respect to R¹.

In alternative embodiments, R¹ and R², together with the atoms to which there are attached, form a 5- to 7-membered nitrogen-containing non-aromatic heterocyclyl, such as a 6-membered nitrogen-containing non-aromatic heterocyclyl, and may be optionally substituted, for example, on a ring nitrogen, with R^{c} where R^{c} is -aliphatic, -aliphatic-amino, —C(O)-aliphatic, or —C(O)-aliphatic-amino, such as —C₆₋₃₀aliphatic, —C₁₋₆aliphatic-amino (for example, —C₁₋₆alkyl-amino), —C(O)—C₆₋₃₀aliphatic or —C(O)—C₁₋₆aliphatic-amino (for example, —C(O)—C₁₋₆alkyl-amino). In any embodiments, R^{c} may comprise an aliphatic moiety that is a straight chain, branched and/or may include a cyclic moiety, for example, a C₅₋₇cyclic moiety, and/or may be saturated or may include 1, 2 or 3 double and/or triple bonds. In certain embodiments, R^{c} is —C₆₋₃₀aliphatic, —C₁₋₆aliphatic-amino, —C(O)—C₆₋₃₀aliphatic or —C(O)—C₁₋₆aliphatic-amino. In certain embodiments, R^{c} is —C(O)—C₆₋₃₀aliphatic. In some embodiments, R^{c} is —C(O)—C₇aliphatic (e.g., caproyl), —C(O)—C₁₁aliphatic (e.g., lauroyl), —C(O)—C₁₅aliphatic (e.g., palmitoyl), —C(O)—C₁₇aliphatic (e.g., stearoyl, oleoyl, or linoleoyl), C₈aliphatic (e.g., capryl), C₁₂aliphatic (e.g., lauryl), C₁₆aliphatic (e.g., (palmityl), or C₁₈aliphatic (e.g., stearyl, oleyl, or linoleyl). In other embodiments, R^{c} is —(CH₂)₁₋₄N(R^{a})₂, where R^{a} is as defined herein.

R³ is —O-aliphatic, such as —O—C₁₋₃₀aliphatic, or N(R⁶)(R⁷) where R⁶ is H or aliphatic, such as H or C₁₋₃₀aliphatic, and R⁷ is aliphatic or C(O)aliphatic, such as C₁₋₃₀aliphatic or C(O)—C₁₋₁₄ aliphatic. In some embodiments, R³ is —O—C₁₋₈aliphatic, such as —O—C₁₋₆aliphatic or C₁₄aliphatic, and/or may be saturated, such as, C₁₋₆alkyl or C₁₄alkyl. In such embodiments, R³ may be a straight chain, branched, or cyclic aliphatic, and in particular embodiments, R³ is methyl, ethyl, propyl, isopropyl or cyclopropyl. In some embodiments, R³ is C₇₋₁₀ aliphatic, C₁₁₋₁₄ aliphatic, C₁₅₋₁₈ aliphatic, C₁₉₋₂₃ aliphatic, or C₂₄₋₃₀ aliphatic. In some embodiments, R³ is —O(C₇₋₁₀ aliphatic), —O(C₁₁ aliphatic), —O(C₁₅₋₁₈ aliphatic), —O(C₁₉₋₂₃ aliphatic), or —O(C₂₄₋₃₀ aliphatic). In some embodiments, the aliphatic of R³ is unsubstituted. In some embodiments, the aliphatic of R³ is substituted at least or only with halogen (e.g., F). In some embodiments, the aliphatic of R³ comprises no unsaturated CC bonds. In some embodiments, the aliphatic of R³ comprises only one unsaturated CC bond. In some embodiments, the aliphatic of R³ comprises only two unsaturated CC bonds. In some embodiments, the aliphatic of R³ is unbranched. In some embodiments, the aliphatic of R³ comprises only one branch. In some embodiments, the aliphatic of R³ comprises only two branches.

In other embodiments, R³ is —O—C₆₋₃₀aliphatic, such as —O—C₆₋₂₅aliphatic, —O—C₈₋₂₀aliphatic, —O—C₁₂₋₂₀aliphatic, —O—C₁₄₋₂₀aliphatic, —O—C₁₆₋₂₀aliphatic, or —O—C₁₆₋₁₈aliphatic, and may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties. In particular examples of such embodiments, R³ is —O—C₁₆₋₂₀alkyl, such as —O—C₁₆₋₁₈alkyl, or —O—C₁₆alkyl, —O—C₁₇alkyl or —O—C₁₈alkyl, or R³ is —O—C₁₆₋₂₀alkenyl, such as —O—C₁₆₋₁₈alkenyl, or —O—C₁₆alkenyl, —O—C₁₇alkenyl or —O—C₁₈alkenyl.

Alternatively, R³ is —N(R⁶)(R⁷). In some embodiments, R⁶ may be H or R⁶ may be C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic, and may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁶ is C₈aliphatic (e.g., capryl), C₁₂aliphatic (e.g., lauryl), C₁₆aliphatic (e.g., (palmityl), or C₁₈aliphatic (e.g., stearyl, oleyl, or linoleyl).

In certain embodiments, the C(O)R³ moiety is C(O)O—C₆₋₃₀aliphatic, and may be a straight chain or branched and/or may include 1, 2 or 3 double bonds. In certain embodiments, R³ is —O— linolenyl.

R⁷ may be aliphatic or —C(O)aliphatic. In some embodiments, R⁷ is C₁₋₈aliphatic, such as C₁₋₆aliphatic or C₁₄aliphatic, but in other embodiments, R⁷ is C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic, and may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁷ is C₈aliphatic (e.g., capryl), C₁₂aliphatic (e.g., lauryl), C₁₆aliphatic (e.g., (palmityl), or C₁₈aliphatic (e.g., stearyl, oleyl, or linoleyl). Alternatively, R⁷ is —C(O)aliphatic, and may be —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic, and may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁷ is —C(O)—C₇aliphatic (e.g., caproyl), —C(O)—C₁₁aliphatic (e.g., lauroyl), —C(O)—C₁₅aliphatic (e.g., palmitoyl), or —C(O)—C₁₇aliphatic (e.g., stearoyl, oleoyl, or linoleoyl).

In any embodiments, R³ may be substituted, such as by amino. In certain embodiments, R³ is N(R⁶)(R⁷). In some embodiments, R³ is N(R⁶)(R⁷), where R⁶ is as defined above, and R⁷ is C₁₋₆aliphatic substituted with amino, such as —(CH₂)₁₋₆N(R^{a})₂ or —(CH₂)₁₋₄N(R^{a})₂, where each R^{a} independently is as defined above with respect to R¹. In certain embodiments, R³ is —NH(substituted C₁₋₆alkyl). In certain embodiments, R³ is —NH(C₁₋₆alkyl substituted at least with —OH, and may be -

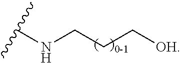

In certain embodiments, R³ is —NH(CH₂)₂₋₄N(C₁₋₆alkyl)₂, and may be —NHCH₂CH₂CH₂NMe₂. In certain embodiments, R³ is —NH(CH₂)₂₋₄N(C₁₋₆alkyl substituted at least with —OH)₂, and may be

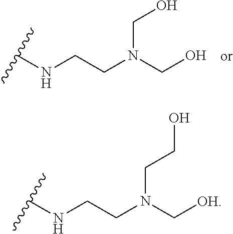 [SMILES: *NCCN(CO)CO ; *NCCN(CO)CCO]

In certain embodiments, R³ is —N[(CH₂)₂₋₄N(C₁₋₆alkyl)₂]₂, and may be

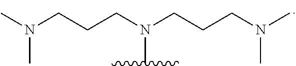

In certain embodiments, R³ is —NH(CH₂)₂₋₄-(5- or 6-membered non-aromatic heterocyclyl), and may be

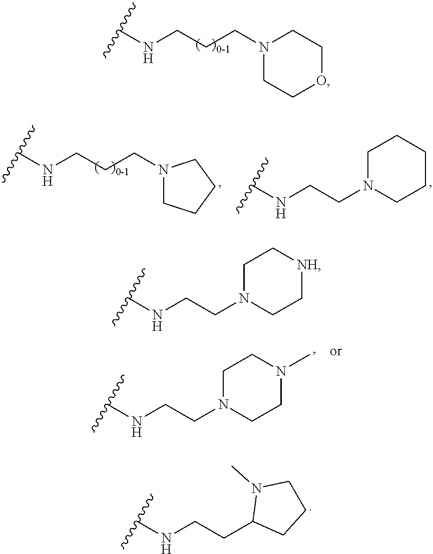

In certain embodiments, R³ is —NH(CH₂)₂₋₄-(5- or 6-membered heteroaryl), and may be

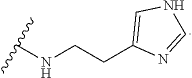 [SMILES: *NCCc1c[nH]cn1]

In certain embodiments, R³ is —NH—(CH₂)ₚ—N(R^{a})₂ or —N[(CH₂)ₚ—N(R^{a})₂]₂, optionally wherein at least one —CH₂— of —(CH₂)ₚ— is replaced with arylene or heteroarylene;
each R^{a} independently is H or C₁₋₆alkyl, or both R^{a}s together with the nitrogen to which they are attached form an 5- or 6-membered heterocyclyl; and
each p is independently an integer from 1 to 8.
   In certain embodiments, R³ is —NH-phenylene-N(R^{a})₂, and may be

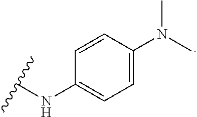 [SMILES: *Nc1ccc(N(C)C)cc1]

In certain embodiments, R³ is N(R⁶)(R⁷), wherein R⁶ and R⁷, together with the atom to which there are attached, form a 4- to 7-membered nitrogen-containing heterocyclyl. In certain embodiments, R³ is N(R⁶)(R⁷), wherein R⁶ and R⁷, together with the atom to which there are attached, form piperazinyl. In certain embodiments, R³ is piperazinyl substituted at least at the 4-position. In certain embodiments, R³ is piperazinyl substituted at least at the 4-position with C₁₋₆ alkyl or 4- to 7-membered heterocyclyl. In certain embodiments, R³ is

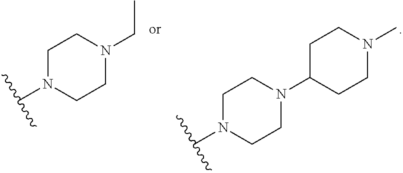

R⁴ is aliphatic or —C(O)aliphatic, such as C₁₋₃₀aliphatic or —C(O)C₁₋₃₀aliphatic, optionally substituted, such as with amino, and R⁵ is H, aliphatic or —C(O)aliphatic, optionally substituted, such as with amino.

R⁴ may be C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic; or —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic. R⁴ may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁴ is —C(O)—C₇aliphatic (e.g., caproyl), —C(O)—C₁₁aliphatic (e.g., lauroyl), —C(O)—C₁₅aliphatic (e.g., palmitoyl), or —C(O)—C₁₇aliphatic (e.g., stearoyl, oleoyl, or linoleoyl).

In other embodiments, R⁴ is C₁₋₈aliphatic, such as C₁₋₆alkyl, C₁₋₄alkyl or C₂₄alkyl; or —C(O)C₁₋₈aliphatic, such as —C(O)C₁₋₆alkyl, —C(O)C₁₋₄alkyl or —C(O)C₂₋₄alkyl. In such embodiments, R⁵ is substituted with N(R^{a})₂, where each R^{a} independently is as defined above with respect to R¹. In some embodiments, R⁴ is —C(O)(CH₂)₁₋₃N(Me)₂.

In some embodiments, R⁵ is H, but in other embodiments, R⁵ is C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic; or —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic. R⁵ may be saturated (i.e., alkyl), or unsaturated (i.e., alkenyl or alkynyl, typically alkenyl with 1, 2, or 3 double bonds), and/or may include cyclic moieties, such as C₅₋₇cycloalkyl moieties. In some embodiments, R⁵ is —C(O)—C₇aliphatic (e.g., caproyl), —C(O)—C₁₁aliphatic (e.g., lauroyl), —C(O)—C₁₅aliphatic (e.g., palmitoyl), or —C(O)—C₁₇aliphatic (e.g., stearoyl, oleoyl, or linoleoyl).

In certain disclosed examples, R⁴ is —C(O)C₁₋₈aliphatic substituted with N(R^{a})₂ and R⁵ is H. In other disclosed examples, R⁴ is —C(O)—C₆₋₃₀aliphatic, and R⁵ is H or —C(O)—C₆₋₃₀aliphatic.

Also with respect to Formula I, the compound comprises at least one C₆₋₃₀aliphatic moiety. In some embodiments of Formula I one of the following conditions applies:
if R² is H, then at least one of R¹, R³, R⁴ and R⁵ is or comprises C₆₋₃₀aliphatic. In certain embodiments, when R² is H, then one or both of R⁴ and R⁵ comprises C₆₋₃₀aliphatic;
if R² is aliphatic, such as C₆₋₃₀aliphatic, then R¹ also is C₆₋₃₀aliphatic; or
if R¹ and R², together with the atoms to which they are attached, form a heterocyclyl moiety, then at least one of R³, R⁴ and R⁵ is or comprises C₆₋₃₀aliphatic, or the heterocyclic moiety formed by R¹ and R² is substituted with a substituent that comprises a C₆₋₃₀aliphatic moiety.

In certain embodiments, the compound has a formula

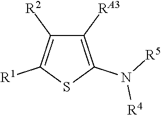

wherein each of R¹ and R² independently is C₆₋₃₀aliphatic.

In certain embodiments, compound has a formula

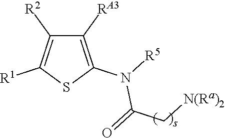

wherein:
each of R¹ and R² independently is C₆₋₃₀aliphatic;
each R^{a} independently is H or C₁₋₆alkyl, or both R^{a}s together with the nitrogen to which they are attached form an optionally substituted 5- or 6-membered heterocyclyl; and s is an integer from 1 to 8.

In certain embodiments of Formula I, X is a bond and the compound has a structure according to Formula I-A:

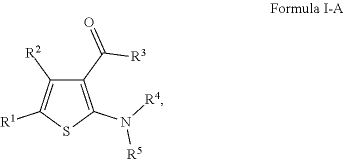

where R¹, R², R³, R⁴ and R⁵ are as defined for Formula I.

In certain embodiments, the compound has a formula

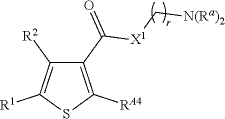

wherein:
each of R¹ and R² independently is C₆₋₃₀aliphatic; and
each R^{a} independently is H or C₁₋₆alkyl, or both R^{a}s together with the nitrogen to which they are attached form an optionally substituted 5- or 6-membered heterocyclyl.

In certain embodiments, the compound has a formula

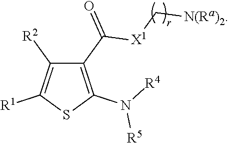

In certain embodiments, the compound has a formula

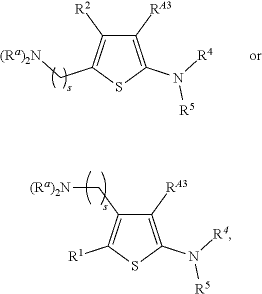

wherein:
each R^{a} independently is H or C₁₋₆alkyl, or both R^{a}s together with the nitrogen to which they are attached form an optionally substituted 5- or 6-membered heterocyclyl; and
s is an integer from 1 to 8.

In certain embodiments, the compound has a formula

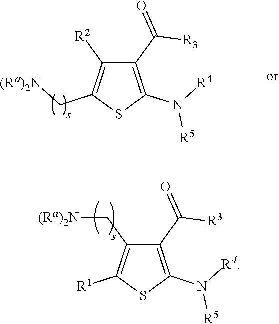

In certain embodiments, s is 2 or 3;
each of R¹ and R² independently is C₆₋₁₀alkyl;
R³ is O-methyl or O-ethyl;
each R^{a} independently is C₁₄alkyl; or
a combination thereof.

In certain embodiments, X is a bond and R¹ and R², together with the atoms to which there are attached, form a 4- to 7-membered nitrogen-containing non-aromatic heterocyclyl; provided that the compound is not of the formula:

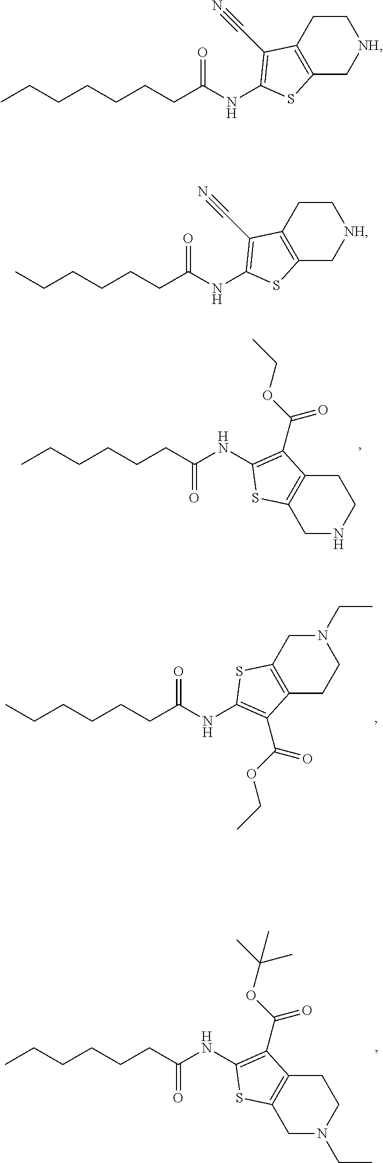

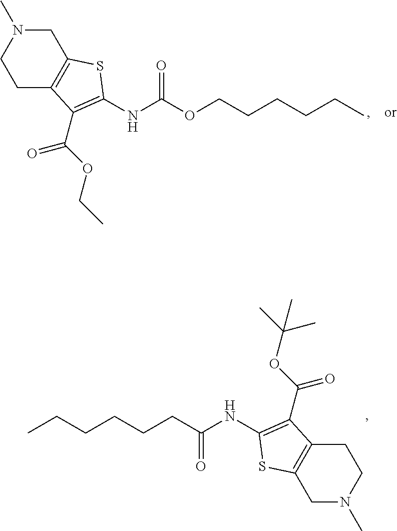

or a salt thereof.

In certain embodiments, X is a bond and R¹ and R², together with the atoms to which there are attached, form pyrrolidinyl, piperidinyl, morpholinyl, or azepanyl. In certain embodiments, X is a bond and R¹ and R², together with the atoms to which there are attached, form pyrrolidinyl, piperidinyl, morpholinyl, or azepanyl, wherein the nitrogen atom of the pyrrolidinyl, piperidinyl, morpholinyl, or azepanyl is substituted with R⁸.

In certain embodiments, the compound has a Formula II′:

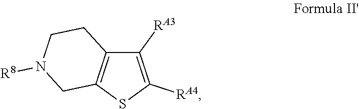

wherein:
R⁸ is H or R^{c}; and
R^{c} is -aliphatic, -aliphatic-amino, —C(O)-aliphatic, or —C(O)-aliphatic-amino.

In some embodiments, R¹ and R², together with the atoms to which they are attached, form a non-aromatic heterocyclyl moiety. In some such embodiments, the compound has a formula II:

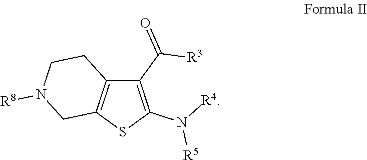

With respect to Formula II, R³, R⁴ and R⁵ are as defined above with respect to Formulas A and I. R⁸ is H or R^{c} where R^{c} is as defined for Formulas A and I. In some embodiments, R⁸ is H. In other embodiments, R⁸ is R^{c} and may be linear or branched —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic, and/or may be saturated (i.e., alkyl), or unsaturated (i.e., alkenyl or alkynyl, typically alkenyl with 1, 2 or 3 double bonds), and/or may include cyclic moieties, such as C₅₋₇cycloalkyl moieties. In some embodiments, R⁸ is C₇₋₁₀ aliphatic, C₁₁₋₁₄ aliphatic, C₁₅₋₁₈ aliphatic, C₁₉₋₂₃ aliphatic, or C₂₄₋₃₀ aliphatic. In some embodiments, R⁸ is —C(O)(C₇₋₁₀ aliphatic), —C(O)(C₁₁₋₁₄ aliphatic), —C(O)(C₁₅₋₁₈ aliphatic), —C(O)(C₁₉₋₂₃ aliphatic), or —C(O)(C₂₄₋₃₀ aliphatic). In some embodiments, R⁸ is unsubstituted. In some embodiments, R⁸ is substituted at least or only with halogen (e.g., F). In some embodiments, R⁸ comprises no unsaturated CC bonds. In some embodiments, R⁸ comprises only one unsaturated CC bond. In some embodiments, R⁸ comprises only two unsaturated CC bonds. In some embodiments, the aliphatic of R⁸ is unbranched. In some embodiments, the aliphatic of R⁸ comprises only one branch. In some embodiments, the aliphatic of R⁸ comprises only two branches.

In some embodiments, R^{c} is C₇₋₁₀ aliphatic, C₁₁₋₁₄ aliphatic, C₁₅₋₁₈ aliphatic, C₁₉₋₂₃ aliphatic, or C₂₄₋₃₀ aliphatic. In some embodiments, R^{c} is —C(O)(C₇₋₁₀ aliphatic), —C(O)(C₁₁₋₁₄ aliphatic), —C(O)(C₁₅₋₁₈ aliphatic), —C(O)(C₁₉₋₂₃ aliphatic), or —C(O)(C₂₄₋₃₀ aliphatic). In some embodiments, the aliphatic of R^{c} is unsubstituted. In some embodiments, the aliphatic of R is substituted at least or only with halogen (e.g., F). In some embodiments, the aliphatic of R^{c} comprises no unsaturated CC bonds. In some embodiments, the aliphatic of R comprises only one unsaturated CC bond. In some embodiments, the aliphatic of R^{c} comprises only two unsaturated CC bonds. In some embodiments, the aliphatic of R^{c} is unbranched. In some embodiments, the aliphatic of R comprises only one branch. In some embodiments, the aliphatic of R^{c} comprises only two branches.

In certain embodiments, the compound has a Formula II-a:

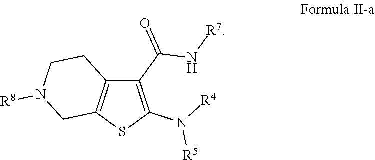

In further embodiments, R⁸ is —C(O)C₁₋₈aliphatic, such as —C(O)C₁₋₆alkyl, —C(O)C₁₋₄alkyl or —C(O)C₂₋₄alkyl, and is substituted with N(R^{a})₂, where each R^{a} independently is as defined above with respect to R¹.

In certain embodiments, the compound has a Formula III′:

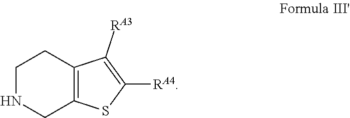

In some embodiments, R⁸ is H, leading to a compound, or salt or solvate thereof, having a Formula III:

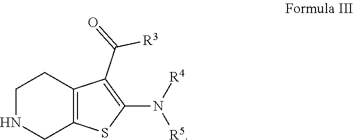

With respect to Formula III, R³, R⁴ and R⁵ are as defined above with respect to Formulas A and I. In some embodiments of Formula III, R³ is —O—C₁₋₆aliphatic, such as —O—C₁₋₆alkyl or —O—C₁₋₄alkyl and may be —O-methyl or —O-ethyl.

In other embodiments of Formula III, R³ is —O—C₆₋₃₀aliphatic, such as —O—C₆₋₂₅aliphatic, —O—C₈₋₂₀aliphatic, —O—C₁₂₋₂₀aliphatic, —O—C₁₄₋₂₀aliphatic, —O—C₁₆₋₂₀aliphatic, or —O—C₁₆₋₁₈aliphatic, and may be saturated (i.e., alkyl), or unsaturated (i.e., alkenyl or alkynyl, typically alkenyl with 1, 2, or double bonds), and/or may include cyclic moieties, such as C₅₋₇cycloalkyl moieties. In particular examples of such embodiments, R³ is —O—C₁₆₋₂₀alkyl, such as —O—C₁₆₋₁₈alkyl, and may be —O—C₁₆alkyl, —O—C₁₇alkyl or —O—C₁₅alkyl; or —O—C₁₆₋₂₀alkenyl, such as —O—C₁₆₋₁₈alkenyl, and may be —O—C₁₆alkenyl, —O—C₁₇alkenyl or —O—C₁₅alkenyl.

In any embodiments of Formula III, R⁴ may be —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic, and may be saturated (i.e., alkyl), or unsaturated (i.e., alkenyl or alkynyl, typically alkenyl with 1, 2 or 3 double bonds), and/or may include cyclic moieties, such as C₅₋₇cycloalkyl moieties.

And R⁵ is H, or R⁵ is —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic, and may be saturated (i.e., alkyl), or unsaturated (i.e., alkenyl or alkynyl, typically alkenyl with 1, 2 or 3 double bonds), and/or may include cyclic moieties, such as C₅₋₇cycloalkyl moieties.

In particular examples of Formula III where R³ is —O—C₁₋₃₀aliphatic, then R⁴ is —C(O)—C₆₋₃₀aliphatic and R⁵ is H.

In some embodiments of Formula III, R³ is —O—C₁₋₆alkyl, such as —O—C₁₋₄alkyl, R⁴ is —C(O)—C₆₋₃₀aliphatic, and R⁵ is H.

In some embodiments of Formula III, R³ is —O—C₁₋₆alkyl, such as —O—C₁₋₄alkyl, and R⁴ and R⁵ are each independently —C(O)—C₆₋₃₀aliphatic.

In certain embodiments, the compound has a Formula IV′

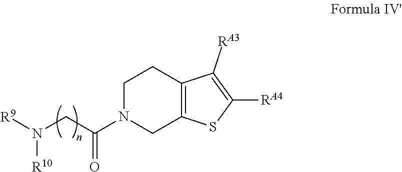

wherein:
each of R⁹ and R¹⁰ independently is H, C₁₋₆alkyl, or R⁹ and R¹⁰ together with the nitrogen to which they are attached, form an optionally substituted 5- or 6-membered non-aromatic heterocyclyl moiety; and
n is an integer from 1 to 8.
   In other embodiments of Formula II, R⁸ is —C(O)C₁₋₈aliphatic substituted with N(R^{a})₂. In some such embodiments, the compound has a structure according to Formula IV:

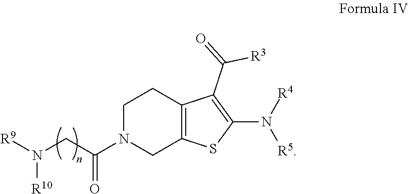

With respect to Formula IV, R³, R⁴ and R⁵ are as defined for Formula II above.

In some embodiments of Formula IV, R³ is —O—C₁₋₈aliphatic, such as —O—C₁₋₆alkyl or —O—C₁₋₄alkyl and may be —O-methyl or —O-ethyl, and at least one of R⁴ and R⁵ comprises C₆₋₃₀aliphatic.

In other embodiments of Formula IV, R³ is —O—C₆₋₃₀aliphatic or N(R⁶)(R⁷) where R⁶ and R⁷ are as defined for Formula I. In some embodiments, R⁶ is C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈ aliphatic, and may be saturated (i.e., alkyl), or unsaturated (i.e., alkenyl or alkynyl, typically alkenyl with 1, 2 or 3 double bonds), and/or may include cyclic moieties, such as C₅₋₇cycloalkyl moieties.

And/or R⁷ is —C(O)aliphatic, and may be —C(O)—C₆₋₃₀aliphatic, such as —C(O)—C₆₋₂₅aliphatic, —C(O)—C₇₋₂₀aliphatic, —C(O)—C₁₁₋₂₀aliphatic, —C(O)—C₁₃₋₂₀aliphatic, —C(O)—C₁₅₋₂₀aliphatic, or —C(O)—C₁₅₋₁₈aliphatic, and may be saturated (i.e., alkyl), or unsaturated (i.e., alkenyl or alkynyl, typically alkenyl with 1, 2 or 3 double bonds), and/or may include cyclic moieties, such as C₅₋₇cycloalkyl moieties.

Alternatively, R⁷ is aliphatic, and may be C₆₋₃₀aliphatic, such as C₆₋₂₅aliphatic, C₈₋₂₀aliphatic, C₁₂₋₂₀aliphatic, C₁₄₋₂₀aliphatic, C₁₆₋₂₀aliphatic, or C₁₆₋₁₈aliphatic, and may be saturated (i.e., alkyl), or unsaturated (i.e., alkenyl or alkynyl, typically alkenyl with 1, 2 or 3 double bonds), and/or may include cyclic moieties, such as C₅₋₇cycloalkyl moieties.

In any embodiments of Formula IV, each of R⁹ and R¹⁰ independently is H, C₁₋₆alkyl, or R⁹ and R¹⁰ together with the nitrogen to which they are attached, form a 5- or 6-membered non-aromatic heterocyclyl moiety, optionally including an additional heteroatom such as O, N or S, and optionally further substituted with a C₁₋₆alkyl.

And n is 1, 2, 3, 4, 5, 6, 7 or 8, such as 1, 2, 3, 4, 5, or 6, or 1, 2, 3, or 4. In certain embodiments, n is 2 or 3. In certain embodiments, each of R⁹ and R¹⁰ independently is C₁₋₆alkyl, such as C₁₄alkyl, and may be methyl or ethyl. In particular embodiments, R⁹ and R¹⁰ are both methyl, and/or n is 1, n is 2 or n is 3.

In some embodiments of Formula IV, R⁴ is —C(O)—C₆₋₃₀aliphatic, such as —C(O)—C₆₋₂₅aliphatic, —C(O)—C₇₋₂₀aliphatic, —C(O)—C₁₁₋₂₀aliphatic, —C(O)—C₁₃₋₂₀aliphatic, —C(O)—C₁₅₋₂₀aliphatic, or —C(O)—C₁₅₋₁₈aliphatic, and may be saturated (i.e., alkyl), or unsaturated (i.e., alkenyl or alkynyl, typically alkenyl, with 1, 2, or 3 double bonds), and/or may include cyclic moieties, such as C₅₋₇cycloalkyl moieties.

In some embodiments of Formula IV, R⁵ is H or —C(O)—C₆₋₃₀aliphatic, such as —C(O)—C₆₋₂₅aliphatic, —C(O)—C₇₋₂₀aliphatic, —C(O)—C₁₁₋₂₀aliphatic, —C(O)—C₁₃₋₂₀aliphatic, —C(O)—C₁₅₋₂₀aliphatic, or —C(O)—C₁₅₋₁₈aliphatic, and may be saturated (i.e., alkyl), or unsaturated (i.e., alkenyl or alkynyl, typically alkenyl, with 1, 2, or 3 double bonds), and/or may include cyclic moieties, such as C₅₋₇cycloalkyl moieties. When R⁴ and R⁵ are both —C(O)—C₆₋₃₀aliphatic, they may be the same or different from each other.

In certain embodiments, the compound has a Formula V′

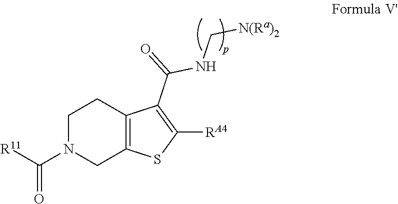

wherein:
R¹¹ is C₆₋₃₀aliphatic;
each R^{a} independently is H or C₁₋₆alkyl, or both R^{a}s together with the nitrogen to which they are attached form an optionally substituted 5- or 6-membered heterocyclyl; and
p is an integer from 1 to 8.

In other embodiments of Formula II, R⁸ is —C(O)C₆₋₃₀aliphatic and R³ is NH(CH₂)₁₋₈N(R^{a})₂. In such embodiments, the compound may have a structure according to Formula V

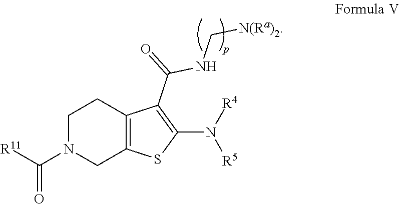

With respect to Formula V, R¹¹ is C₆₋₃₀aliphatic, such as —C₆₋₂₅aliphatic, —C₇₋₂₀aliphatic, —C₁₁₋₂₀aliphatic, —C₁₃₋₂₀aliphatic, —C₁₅₋₂₀aliphatic, or —C₁₅₋₁₈aliphatic, and may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties.

p is from 1 to 8, such as 1, 2, 3, 4, 5, or 6, or 1, 2, 3, or 4.

Each R^{a} independently is as defined above for Formula I. In some embodiments, each R^{a} independently is C₁₋₆alkyl, and may be methyl or ethyl. In some embodiments, each R^{a} independently is unsubstituted C₁₋₆alkyl. In some embodiments, each R^{a} independently is unsubstituted C₁₋₃alkyl.

Also with respect to Formula V, R⁴ is —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic, and may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties.

And R⁵ is H or —C(O)—C₆₋₃₀aliphatic, such as —C(O)—C₆₋₂₅aliphatic, —C(O)—C₈₋₂₀aliphatic, —C(O)—C₁₂₋₂₀aliphatic, —C(O)—C₁₄₋₂₀aliphatic, —C(O)—C₁₆₋₂₀aliphatic, or —C(O)—C₁₆₋₁₈aliphatic, and may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties. When R⁴ and R⁵ are both —C(O)—C₆₋₃₀aliphatic they may be the same or different from each other.

In alternative embodiments of Formula I, R² is H, leading to compounds according to Formula VI:

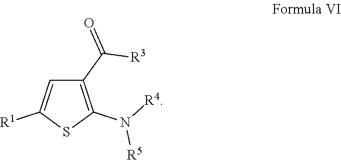

With respect to Formula VI, R¹, R³, R⁴ and R⁵ are as defined for Formulas A and I. In some embodiments, R¹ is alkyl substituted with amino, such as —C₁₋₆alkyl-N(R^{a})₂, or C₁₋₄alkyl-N(R^{a})₂, where each R^{a} independently is as defined above for Formulas A and I. In some embodiments, each R^{a} independently is C₁₋₆alkyl, and may be methyl or ethyl. In other embodiments, each R^{a} is H. And in certain embodiments, the compound has a Formula VII:

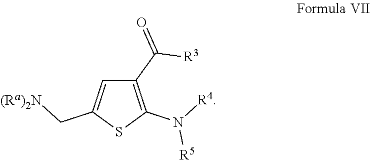

With respect to Formula VII, R^{a}, R³, R⁴ and R⁵ are as defined for Formula VI. In some embodiments of Formulas VI and VII, each R^{a} is H, R³ is —O—C₁₋₆alkyl, R⁴ is —C(O)—C₆₋₃₀aliphatic, and R⁵ is H or —C(O)—C₆₋₃₀aliphatic and may be the same or different from R⁴.

In other embodiments of Formulas A and I, the compound has a structure according to Formula VIII:

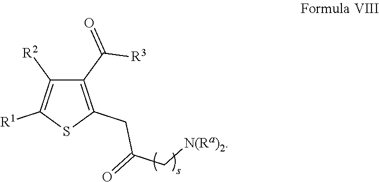

With respect to Formula VIII, each of R¹ and R² independently is C₆₋₃₀aliphatic, C₆₋₂₅aliphatic, C₆₋₂₀aliphatic, C₆₋₁₈aliphatic, C₆₋₁₆aliphatic, C₆₋₁₄aliphatic, or C₆₋₁₀aliphatic, and may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties.

R³ is —O—C₁₋₈aliphatic, such as —O—C₁₋₆aliphatic, or —O—C₁₋₄aliphatic, and may be saturated, such as, —O—C₁₋₆alkyl or —O—C₁₋₄alkyl. In some embodiments, R³ is —O-methyl or —O-ethyl.

s is from 1 to 8, such as 1, 2, 3, 4, 5, or 6; 1, 2, 3, or 4; and may be 2 or 3.

Each R^{a} independently is as defined above for Formulas A and I. In some embodiments, each R^{a} independently is C₁₋₆alkyl, such as C₁₄alkyl, and may be methyl or ethyl.

In some other embodiments of Formulas A and I, the compound has a structure according to Formula IX:

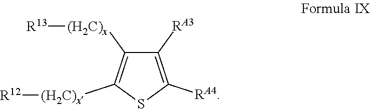

With respect to Formula IX, each of R¹² and R¹³ independently is —C(O)O—R^{f}, —OC(O)—R^{f}, —C(O)NH—R^{f}, or —NH(C(O)—R^{f}, where each R^{f} independently is a linear or branched C₆₋₃₀aliphatic, such as C₆₋₂₅aliphatic, C₆₋₂₀aliphatic, C₆₋₁₈aliphatic, C₆₋₁₆aliphatic, C₆₋₁₄aliphatic, or C₆₋₁₀aliphatic, and R^{f} may be saturated (i.e., alkyl), or unsaturated (i.e., alkenyl or alkynyl, typically alkenyl, with 1, 2 or 3 double bonds), and/or may include cyclic moieties, such as C₅₋₇cycloalkyl moieties. In certain embodiments, R^{f} one or both R^{f} moieties are branched.

Each of x and x′ independently is from 1 to 8, such as 1, 2, 3, 4, 5, or 6; 1, 2, 3, or 4; and may be 2 or 3. In some embodiments, x and x′ are the same but in other embodiments, x and x′ are different.

R^{A3} is —C(O)R³ or —X¹(CH₂)ᵣN(R^{a})₂.

R^{A4} is —NH₂, —NHC(O)(CH₂)₁₋₈N(R^{a})₂, —NH(CH₂)₁₋₈N(R^{a})₂, or —N═C(CH₂)₁₋₈N(R^{a})₂.

And each R³, X¹, r, and R^{a} independently are as previously defined for Formula A

In other embodiments of Formula A, the compound has a formula selected from:

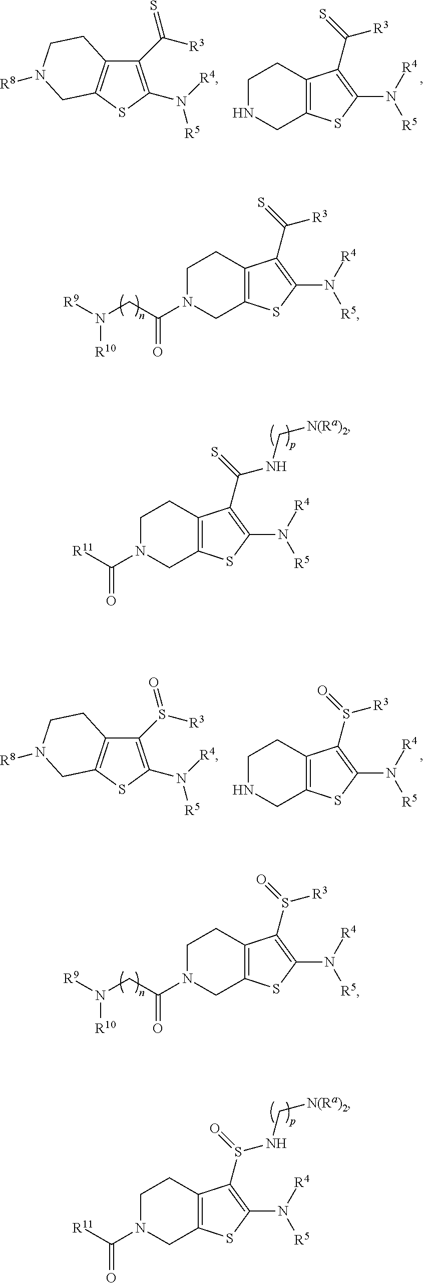

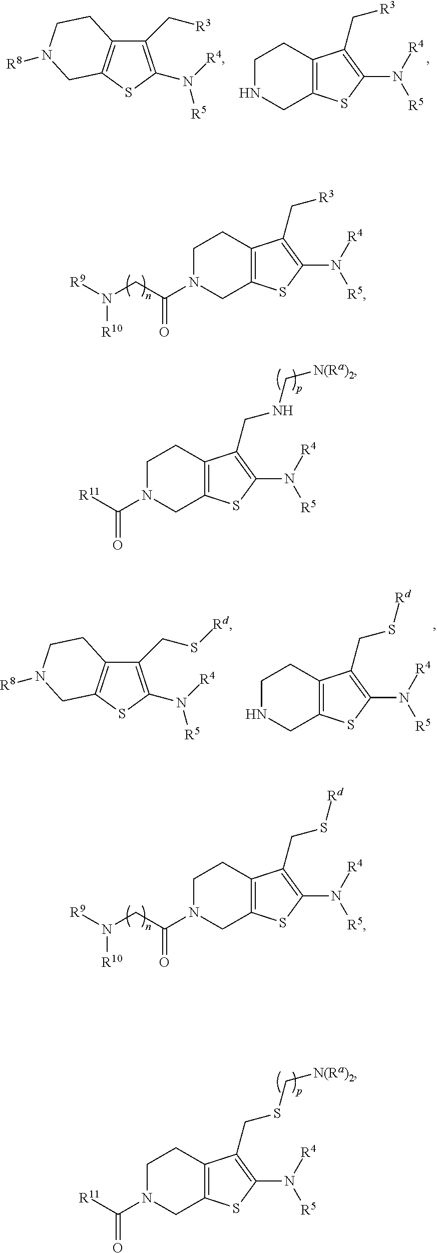

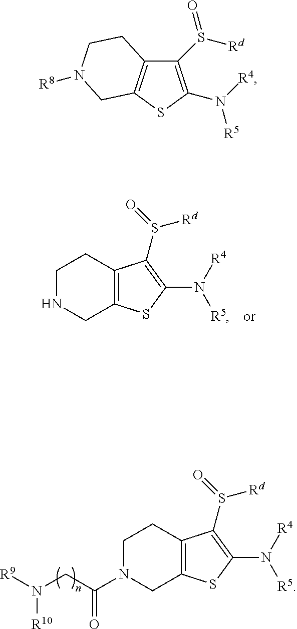

With respect to these formulas, R³, R⁴, R⁵, R⁸, R⁹, R¹⁰, R¹¹, R^{a}, R^{d}, n, and p are as defined herein, such as for Formulas A, I and II-V.

In certain embodiments, the compound comprises one or more basic nitrogen atoms. In certain embodiments, the compound comprises only one basic nitrogen atom. In certain embodiments, the compound comprises only two basic nitrogen atoms. In certain embodiments, the compound comprises —C(R^{a})₂—NH₂, —C(R^{a})₂—NH—C(R^{a})₂—, or

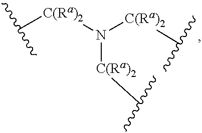

and at least one (e.g., each) of the basic nitrogen atoms is the nitrogen atom in —C(R^{a})₂—NH₂, —C(R^{a})₂—NH—C(R^{a})₂—, or

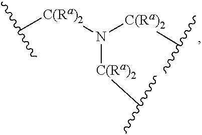

wherein each instance of R^{a} is independently H or C₁₋₆alkyl. In certain embodiments, each instance of C₁₋₆aliphatic and C₁₋₆alkyl is unsubstituted C₁₋₅alkyl (e.g., unsubstituted C₁₋₃alkyl).

In certain embodiments, the compound comprises -(substituted or unsubstituted aryl or substituted or unsubstituted heteroaryl)-NH₂, -(substituted or unsubstituted aryl or substituted or unsubstituted heteroaryl)-NH—C(R^{a})₂—, or

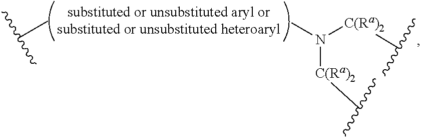

and at least one (e.g., each) of the basic nitrogen atoms is the nitrogen atom in -(substituted or unsubstituted aryl or substituted or unsubstituted heteroaryl)-NH₂, -(substituted or unsubstituted aryl or substituted or unsubstituted heteroaryl)-NH—C(R^{a})₂—, or

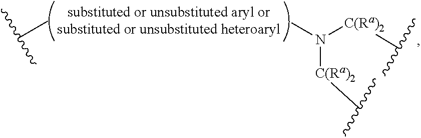

wherein each instance of R^{a} is independently H or C₁₋₆alkyl. In certain embodiments, each instance of C₁₋₆aliphatic and C₁₋₆alkyl is unsubstituted C₁₋₅alkyl (e.g., unsubstituted C₁₋₃alkyl).

In certain embodiments, the compound comprises at least one substituted or unsubstituted, non-aromatic heterocyclyl comprising at least one nitrogen atom in the non-aromatic heterocyclic ring system, and at least one (e.g., each) of the basic nitrogen atoms is the at least one nitrogen atom in the non-aromatic heterocyclic ring system. In certain embodiments, the non-aromatic heterocyclyl is pyrrolidinyl, piperidinyl, piperazinyl, or morpholinyl.

In certain embodiments, the compound comprises at least one substituted or unsubstituted heteroaryl comprising at least one nitrogen atom in the heteroaryl ring system, and at least one (e.g., each) of the basic nitrogen atoms is the at least one nitrogen atom in the heteroaryl ring system. In certain embodiments, the heteroaryl is imidazolyl.

In certain embodiments, the compound does not comprise an acidic moiety. In certain embodiments, the acidic moiety is —C(O)OH, —S(O)OH, —S(O)₂OH, or —P(O)(OH)₂.

Exemplary compounds according to the disclosure include, but are not limited to:

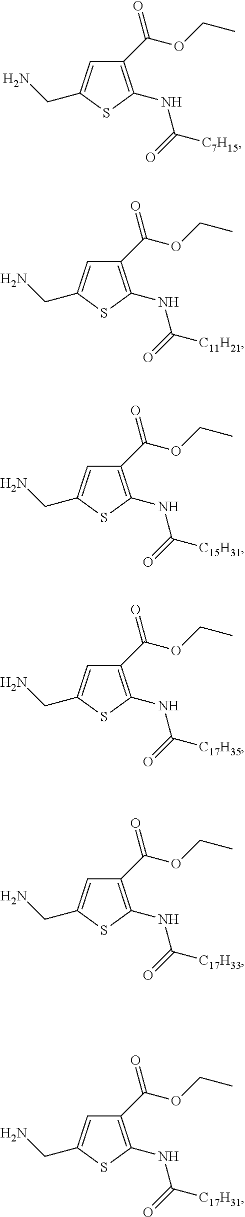

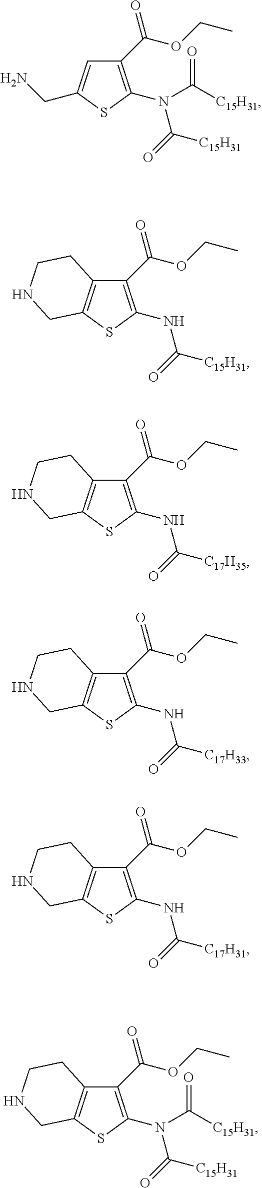

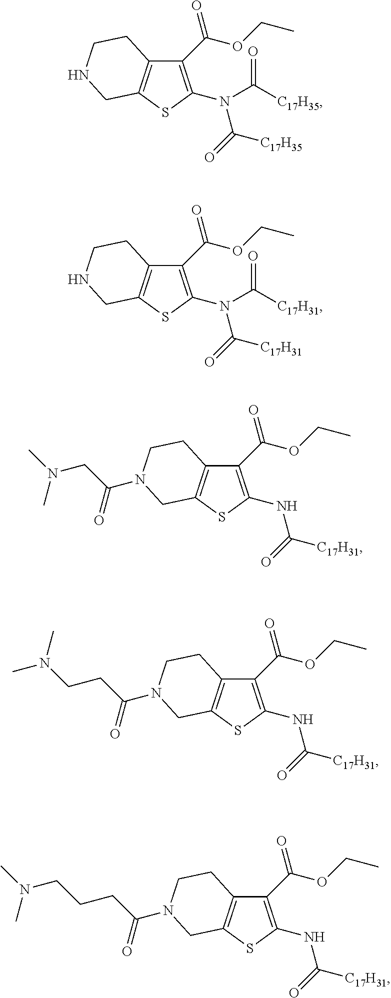

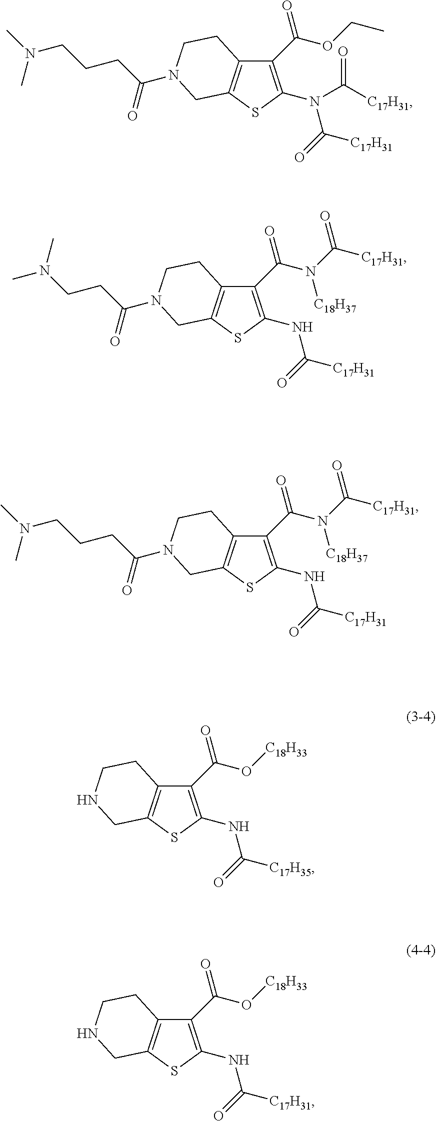

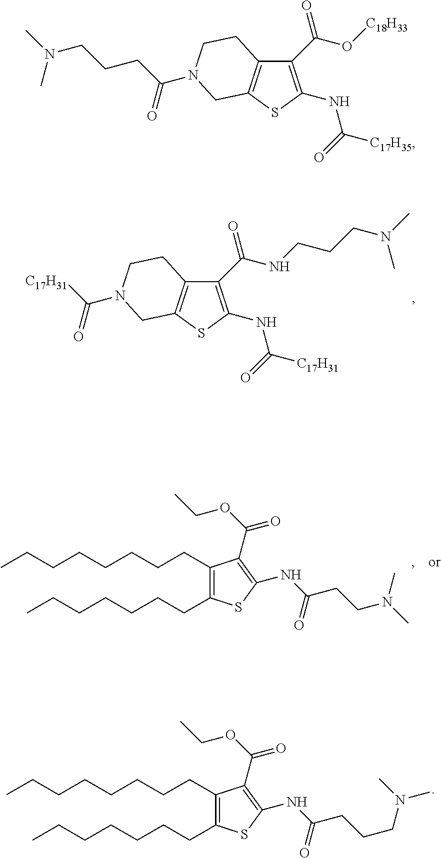

With respect to the compounds above, unless otherwise specified, the C₇, C₁₁, C₁₅, and C₁₇ moieties are linear or branched. In some embodiments, these moieties are linear, but in other embodiments, these moieties are branched.

In certain embodiments, the compound is of the formula:

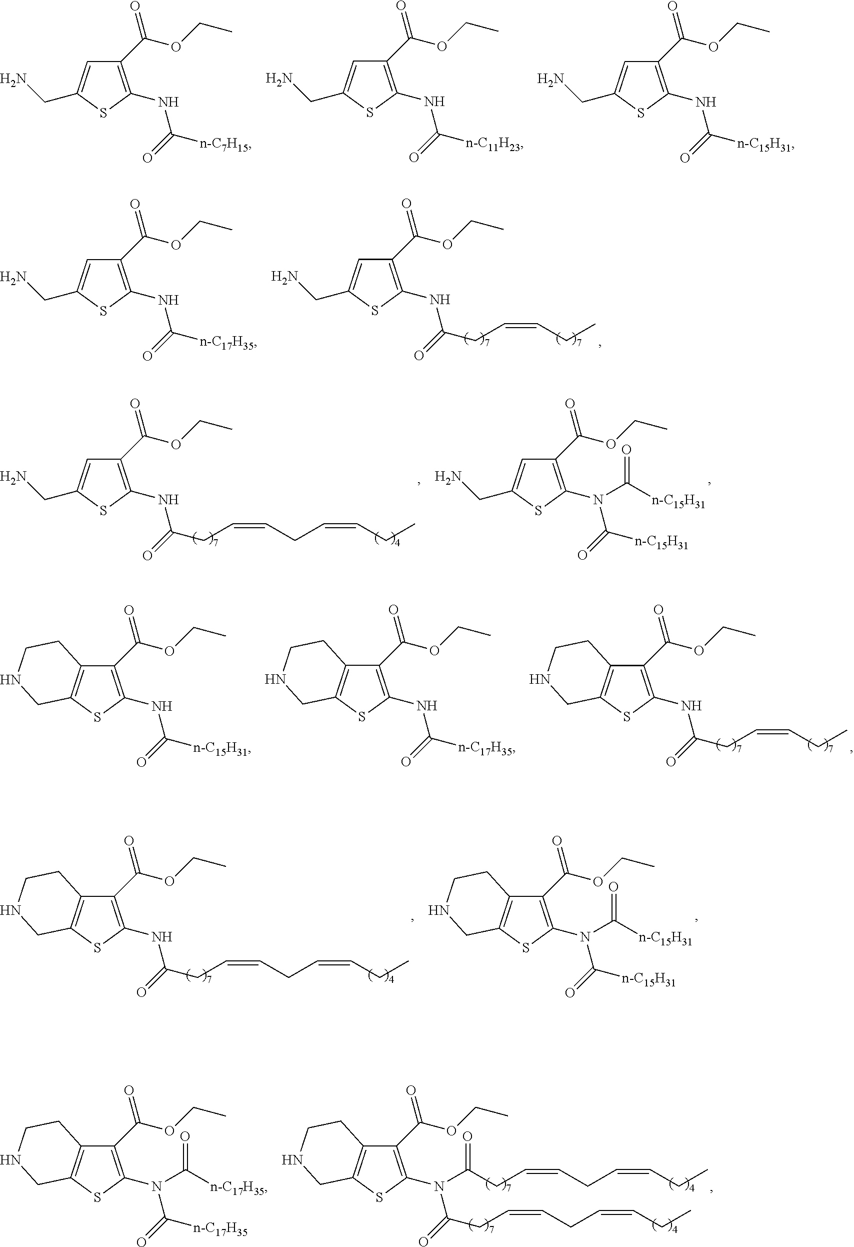

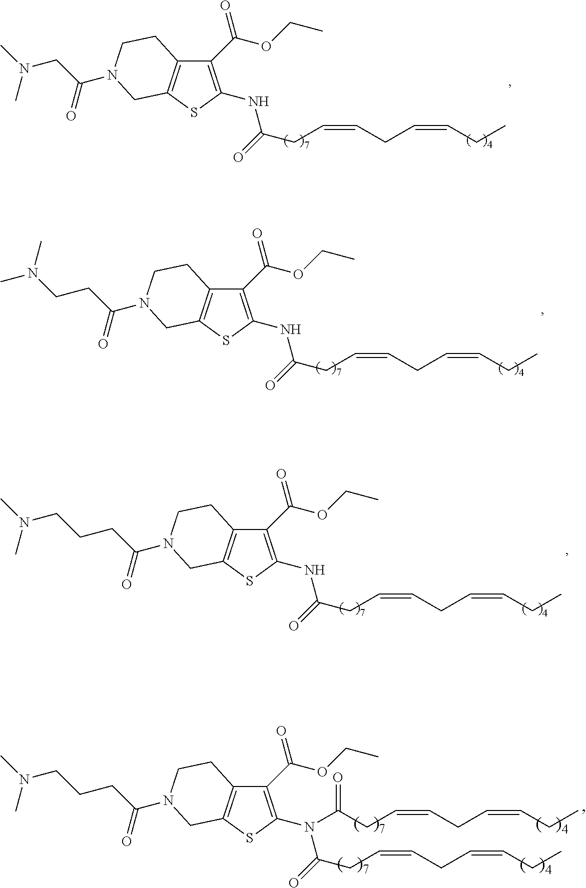

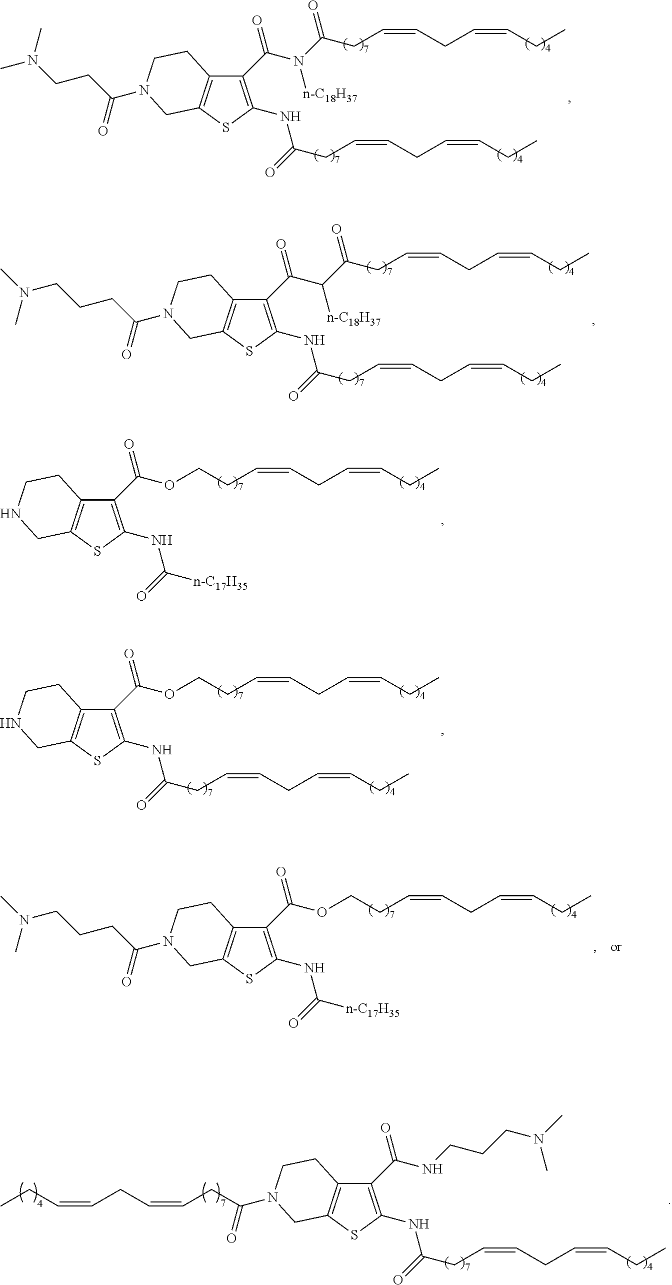

Additional exemplary compounds are shown in Table 1.

TABLE 1

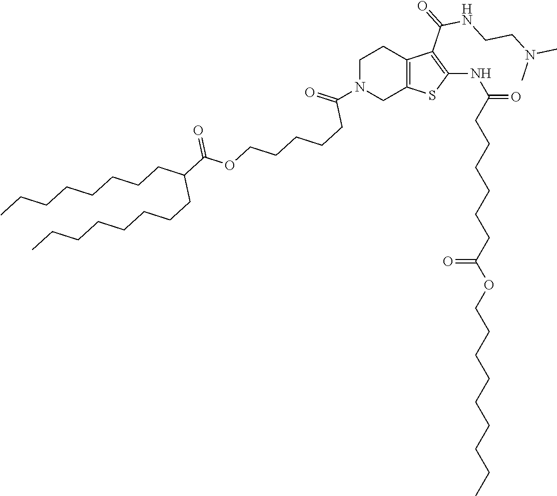 [SMILES: CCCCCCCCCOC(=O)CCCCCCC(=O)Nc1sc2c(c1C(=O)NCCN(C)C)CCN(C(=O)CCCCCOC(=O)C(CCCCCCCC)CCCCCCCC)C2]

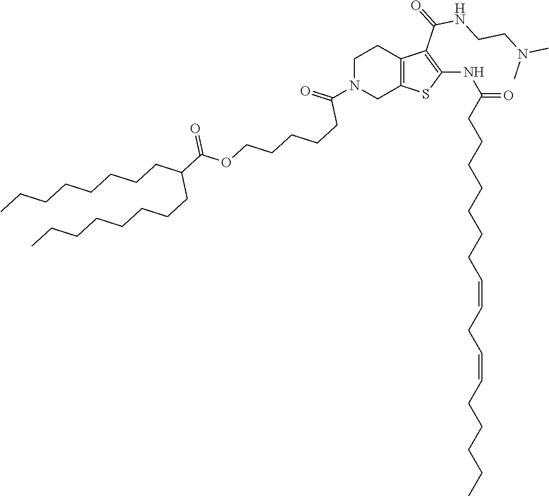 [SMILES: CCCCC/C=C\C/C=C\CCCCCCCC(=O)Nc1sc2c(c1C(=O)NCCN(C)C)CCN(C(=O)CCCCCOC(=O)C(CCCCCCCC)CCCCCCCC)C2]

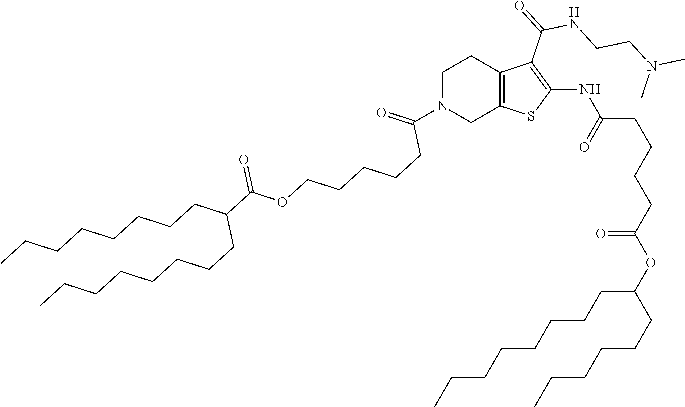 [SMILES: CCCCCCCCC(CCCCCC)OC(=O)CCCCC(=O)Nc1sc2c(c1C(=O)NCCN(C)C)CCN(C(=O)CCCCCOC(=O)C(CCCCCCCC)CCCCCCCC)C2]

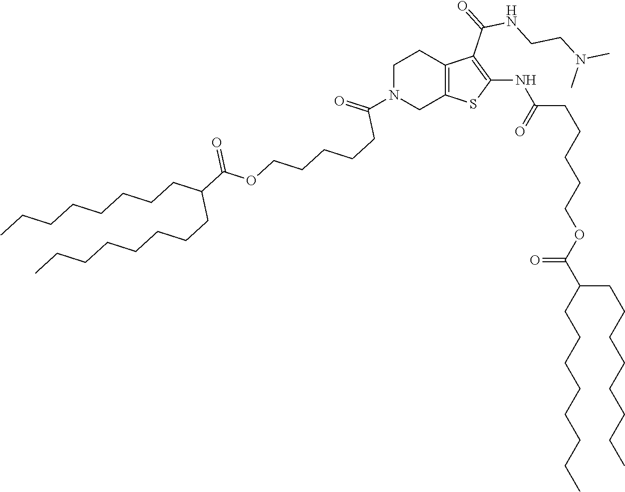 [SMILES: CCCCCCCCC(CCCCCCCC)C(=O)OCCCCCC(=O)Nc1sc2c(c1C(=O)NCCN(C)C)CCN(C(=O)CCCCCOC(=O)C(CCCCCCCC)CCCCCCCC)C2]

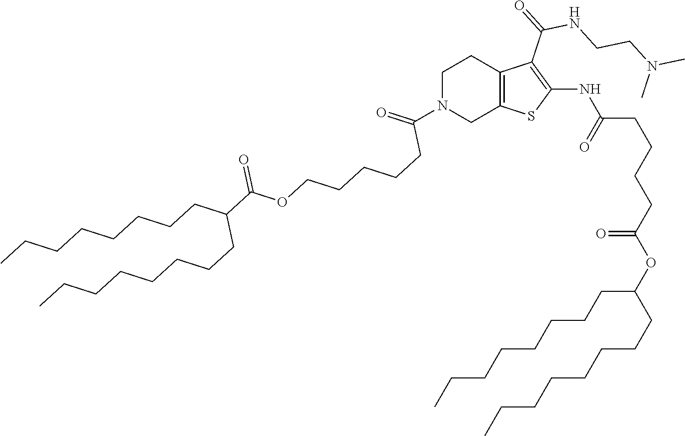 [SMILES: CCCCCCCCC(CCCCCCCC)OC(=O)CCCCC(=O)Nc1sc2c(c1C(=O)NCCN(C)C)CCN(C(=O)CCCCCOC(=O)C(CCCCCCCC)CCCCCCCC)C2]

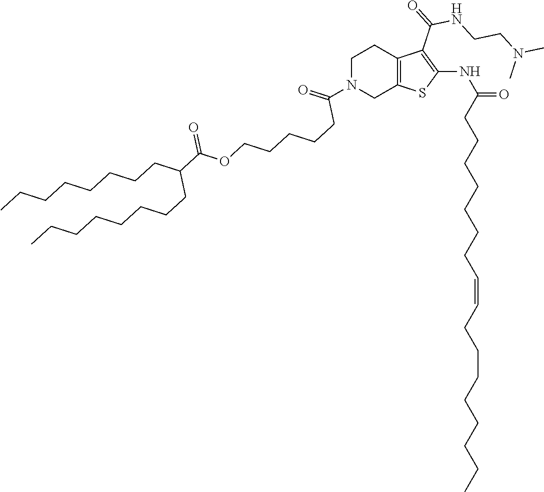 [SMILES: CCCCCCCC/C=C\CCCCCCCC(=O)Nc1sc2c(c1C(=O)NCCN(C)C)CCN(C(=O)CCCCCOC(=O)C(CCCCCCCC)CCCCCCCC)C2]

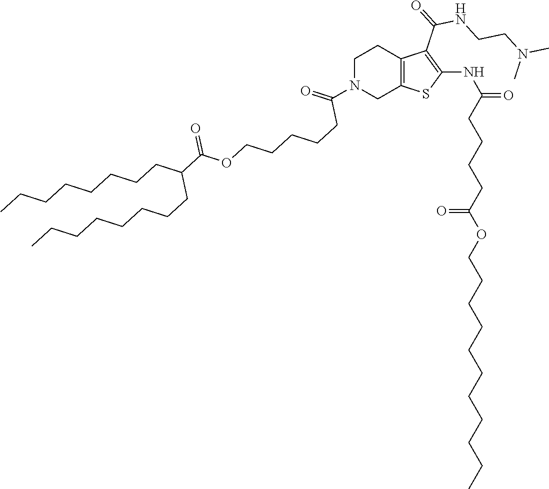 [SMILES: CCCCCCCCCCCOC(=O)CCCCC(=O)Nc1sc2c(c1C(=O)NCCN(C)C)CCN(C(=O)CCCCCOC(=O)C(CCCCCCCC)CCCCCCCC)C2]

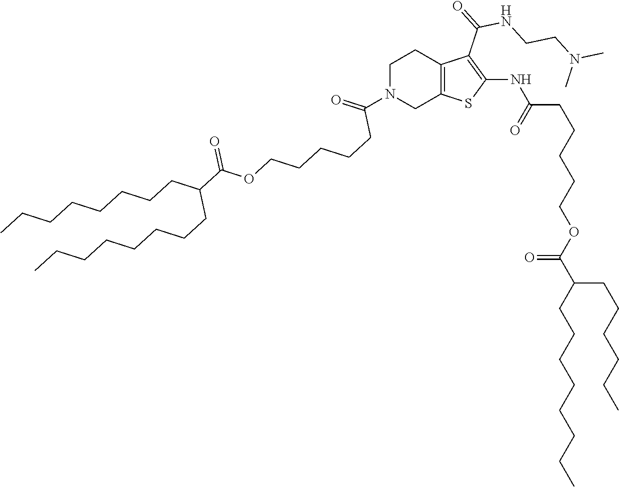 [SMILES: CCCCCCCCC(CCCCCC)C(=O)OCCCCCC(=O)Nc1sc2c(c1C(=O)NCCN(C)C)CCN(C(=O)CCCCCOC(=O)C(CCCCCCCC)CCCCCCCC)C2]

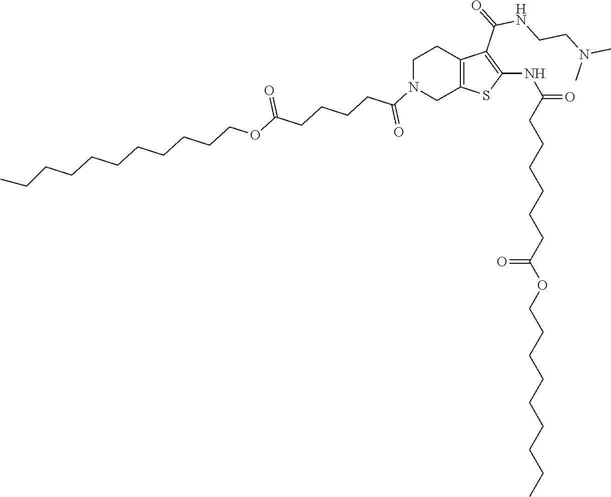 [SMILES: CCCCCCCCCCCOC(=O)CCCCC(=O)N1CCc2c(sc(NC(=O)CCCCCCC(=O)OCCCCCCCCC)c2C(=O)NCCN(C)C)C1]

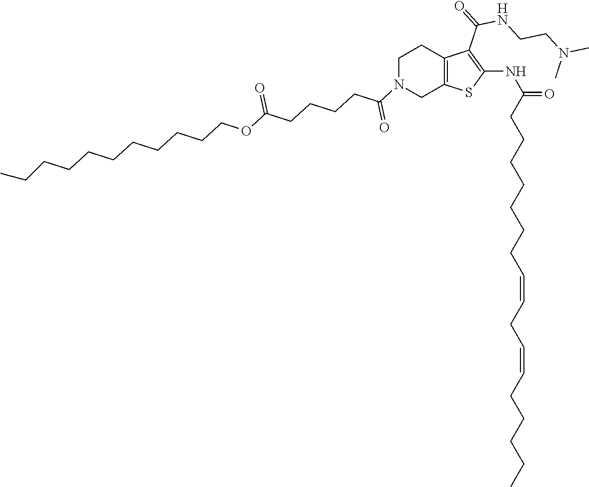 [SMILES: CCCCC/C=C\C/C=C\CCCCCCCC(=O)Nc1sc2c(c1C(=O)NCCN(C)C)CCN(C(=O)CCCCC(=O)OCCCCCCCCCCC)C2]

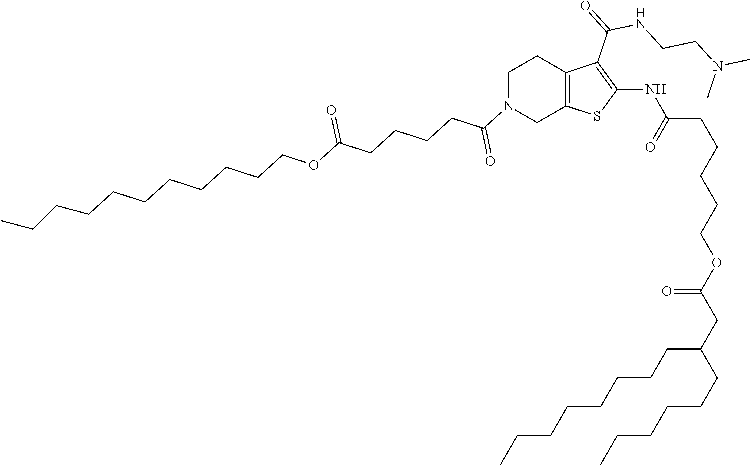 [SMILES: CCCCCCCCCCCOC(=O)CCCCC(=O)N1CCc2c(sc(NC(=O)CCCCCOC(=O)CC(CCCCCC)CCCCCCCC)c2C(=O)NCCN(C)C)C1]

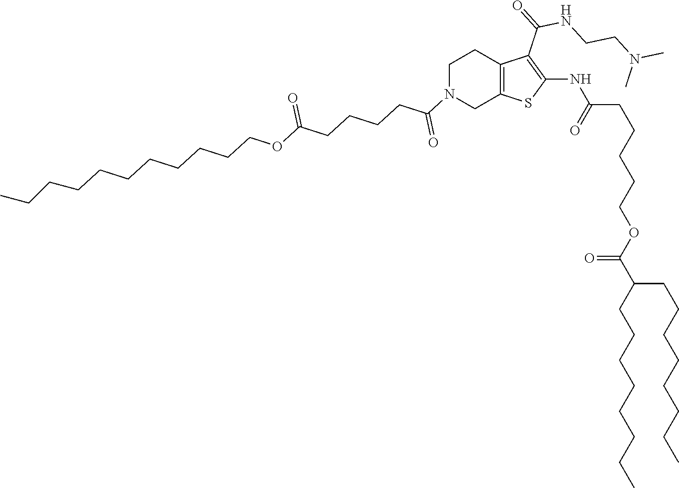 [SMILES: CCCCCCCCCCCOC(=O)CCCCC(=O)N1CCc2c(sc(NC(=O)CCCCCOC(=O)C(CCCCCCCC)CCCCCCCC)c2C(=O)NCCN(C)C)C1]

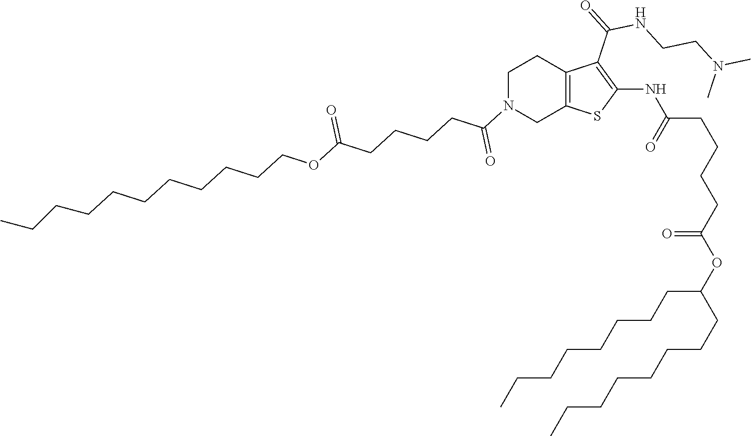 [SMILES: CCCCCCCCCCCOC(=O)CCCCC(=O)N1CCc2c(sc(NC(=O)CCCCC(=O)OC(CCCCCCCC)CCCCCCCC)c2C(=O)NCCN(C)C)C1]

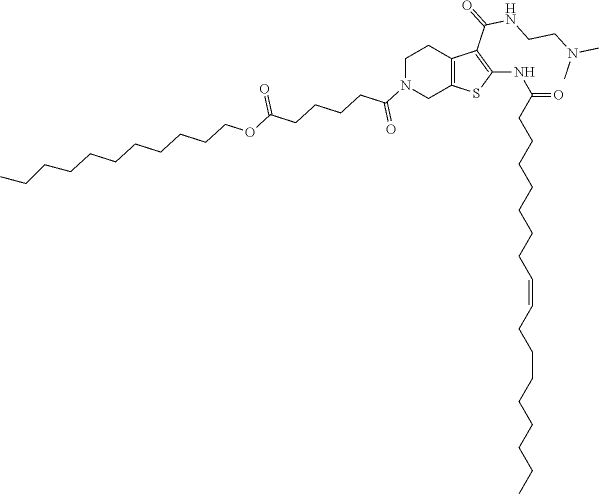 [SMILES: CCCCCCCC/C=C\CCCCCCCC(=O)Nc1sc2c(c1C(=O)NCCN(C)C)CCN(C(=O)CCCCC(=O)OCCCCCCCCCCC)C2]

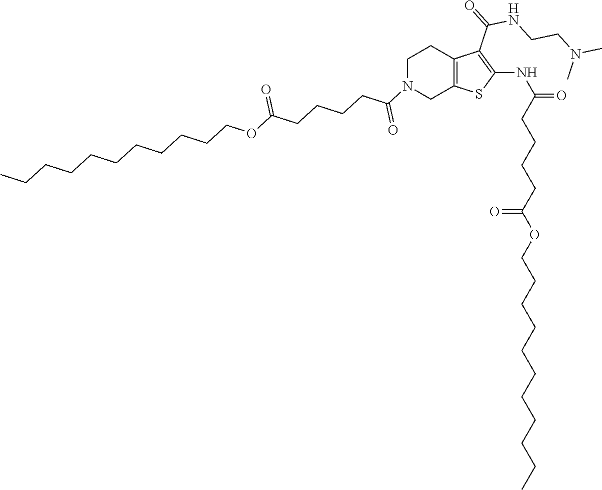 [SMILES: CCCCCCCCCCCOC(=O)CCCCC(=O)Nc1sc2c(c1C(=O)NCCN(C)C)CCN(C(=O)CCCCC(=O)OCCCCCCCCCCC)C2]

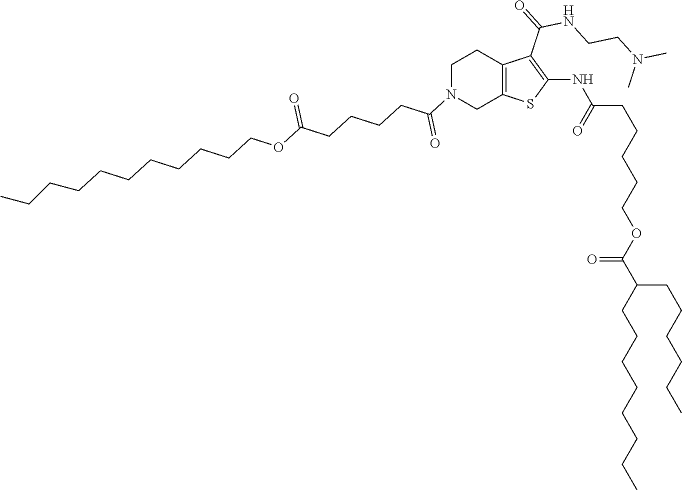 [SMILES: CCCCCCCCCCCOC(=O)CCCCC(=O)N1CCc2c(sc(NC(=O)CCCCCOC(=O)C(CCCCCC)CCCCCCCC)c2C(=O)NCCN(C)C)C1]

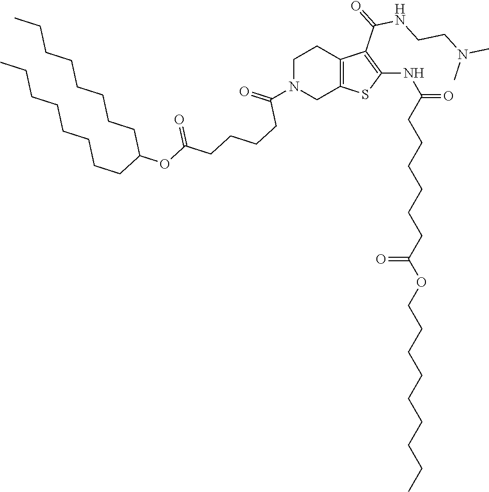 [SMILES: CCCCCCCCCOC(=O)CCCCCCC(=O)Nc1sc2c(c1C(=O)NCCN(C)C)CCN(C(=O)CCCCC(=O)OC(CCCCCCCC)CCCCCCCC)C2]

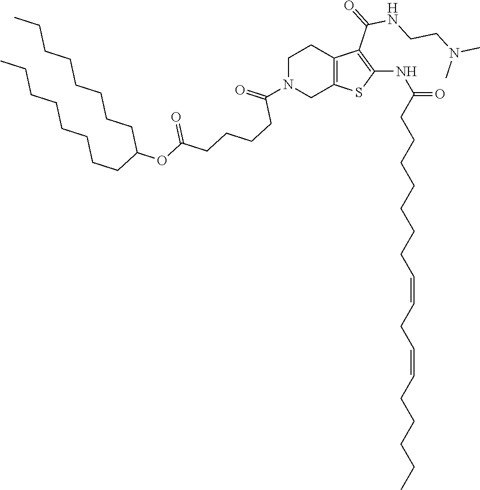 [SMILES: CCCCC/C=C\C/C=C\CCCCCCCC(=O)Nc1sc2c(c1C(=O)NCCN(C)C)CCN(C(=O)CCCCC(=O)OC(CCCCCCCC)CCCCCCCC)C2]

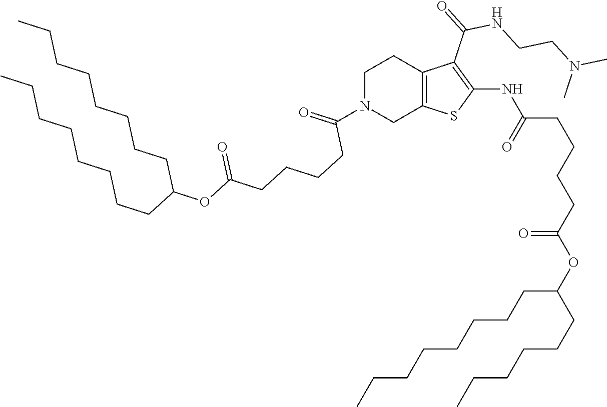 [SMILES: CCCCCCCCC(CCCCCC)OC(=O)CCCCC(=O)Nc1sc2c(c1C(=O)NCCN(C)C)CCN(C(=O)CCCCC(=O)OC(CCCCCCCC)CCCCCCCC)C2]

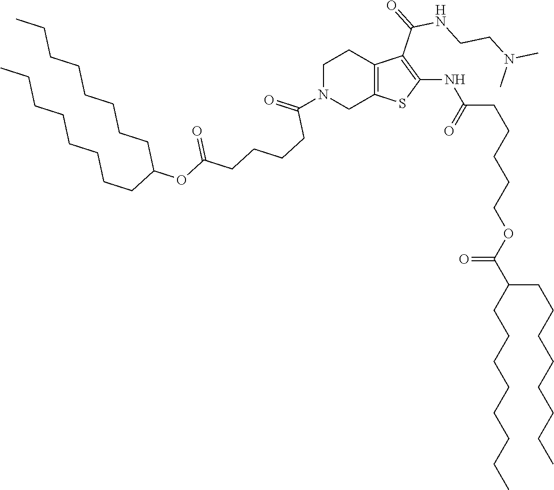 [SMILES: CCCCCCCCC(CCCCCCCC)OC(=O)CCCCC(=O)N1CCc2c(sc(NC(=O)CCCCCOC(=O)C(CCCCCCCC)CCCCCCCC)c2C(=O)NCCN(C)C)C1]

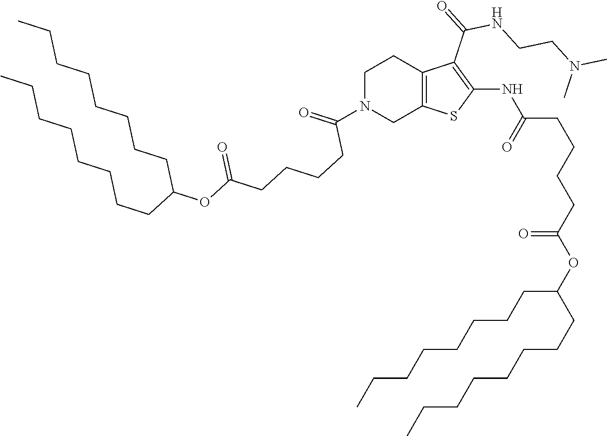 [SMILES: CCCCCCCCC(CCCCCCCC)OC(=O)CCCCC(=O)Nc1sc2c(c1C(=O)NCCN(C)C)CCN(C(=O)CCCCC(=O)OC(CCCCCCCC)CCCCCCCC)C2]

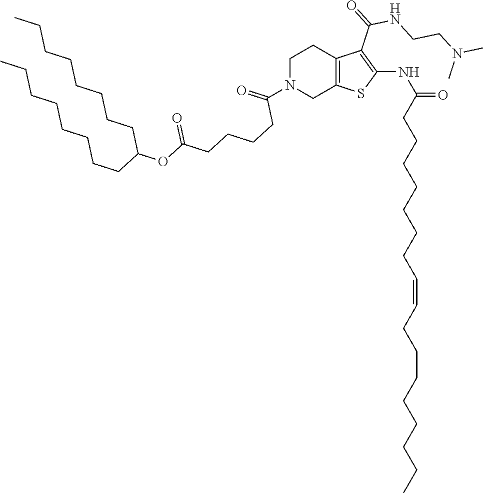 [SMILES: CCCCCCCC/C=C\CCCCCCCC(=O)Nc1sc2c(c1C(=O)NCCN(C)C)CCN(C(=O)CCCCC(=O)OC(CCCCCCCC)CCCCCCCC)C2]

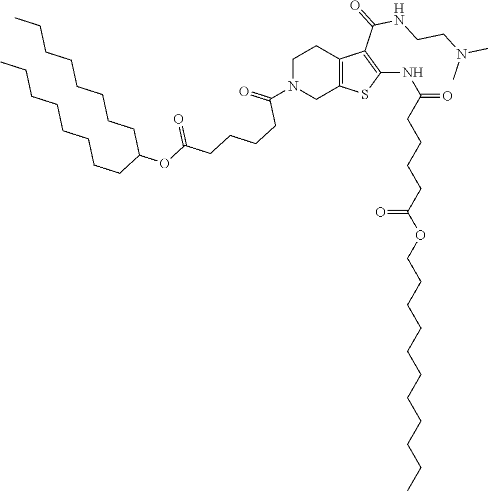 [SMILES: CCCCCCCCCCCOC(=O)CCCCC(=O)Nc1sc2c(c1C(=O)NCCN(C)C)CCN(C(=O)CCCCC(=O)OC(CCCCCCCC)CCCCCCCC)C2]

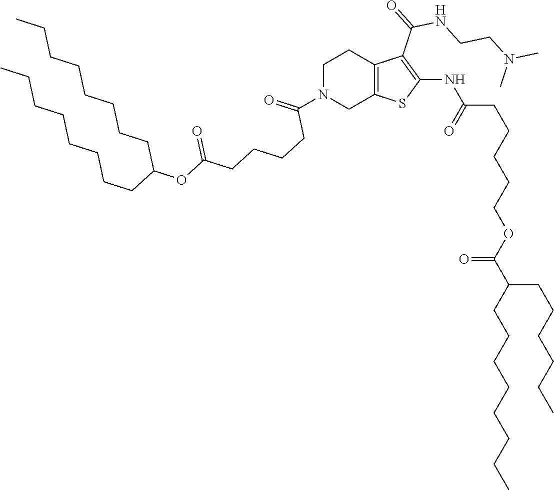 [SMILES: CCCCCCCCC(CCCCCCCC)OC(=O)CCCCC(=O)N1CCc2c(sc(NC(=O)CCCCCOC(=O)C(CCCCCC)CCCCCCCC)c2C(=O)NCCN(C)C)C1]

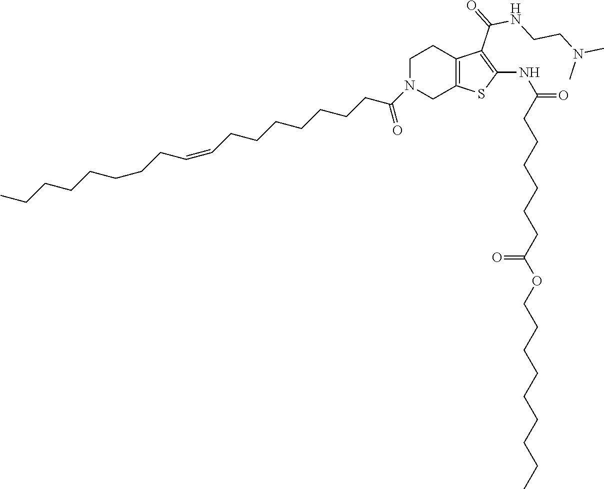 [SMILES: CCCCCCCC/C=C\CCCCCCCC(=O)N1CCc2c(sc(NC(=O)CCCCCCC(=O)OCCCCCCCCC)c2C(=O)NCCN(C)C)C1]

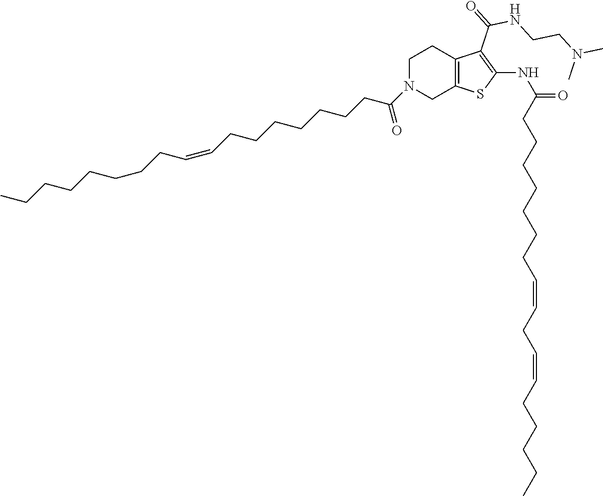 [SMILES: CCCCC/C=C\C/C=C\CCCCCCCC(=O)Nc1sc2c(c1C(=O)NCCN(C)C)CCN(C(=O)CCCCCCC/C=C\CCCCCCCC)C2]

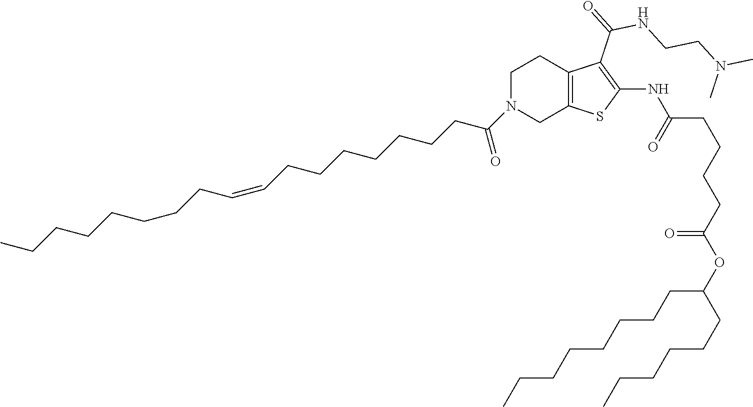 [SMILES: CCCCCCCC/C=C\CCCCCCCC(=O)N1CCc2c(sc(NC(=O)CCCCC(=O)OC(CCCCCC)CCCCCCCC)c2C(=O)NCCN(C)C)C1]

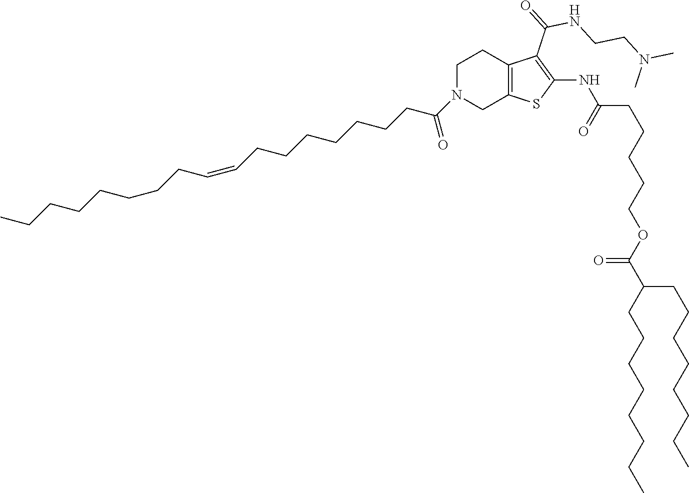 [SMILES: CCCCCCCC/C=C\CCCCCCCC(=O)N1CCc2c(sc(NC(=O)CCCCCOC(=O)C(CCCCCCCC)CCCCCCCC)c2C(=O)NCCN(C)C)C1]

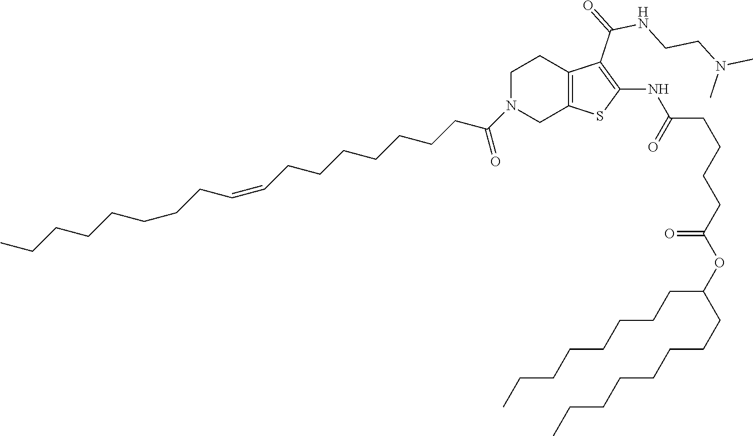 [SMILES: CCCCCCCC/C=C\CCCCCCCC(=O)N1CCc2c(sc(NC(=O)CCCCC(=O)OC(CCCCCCCC)CCCCCCCC)c2C(=O)NCCN(C)C)C1]

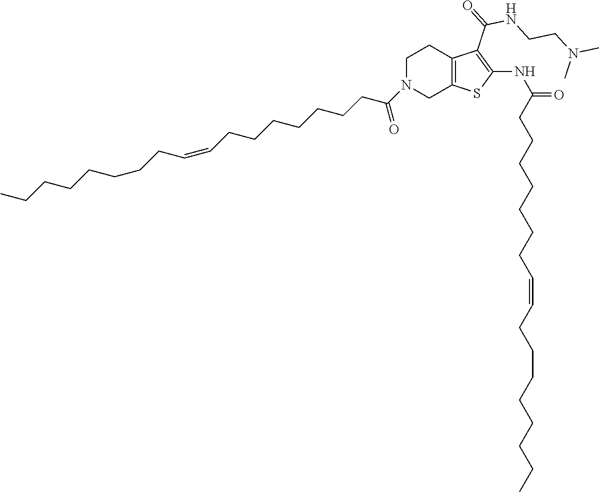 [SMILES: CCCCCCCC/C=C\CCCCCCCC(=O)Nc1sc2c(c1C(=O)NCCN(C)C)CCN(C(=O)CCCCCCC/C=C\CCCCCCCC)C2]

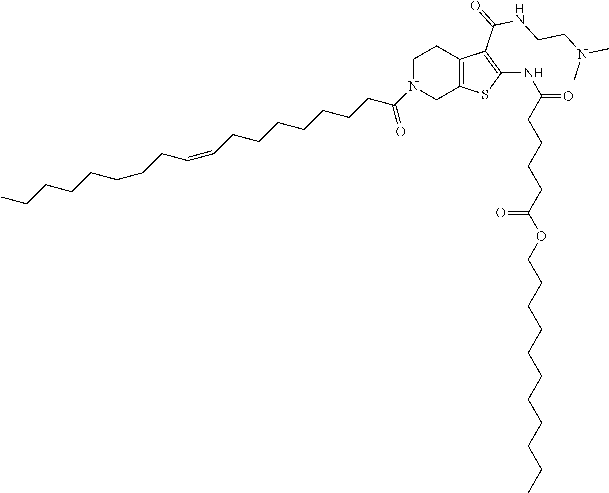 [SMILES: CCCCCCCC/C=C\CCCCCCCC(=O)N1CCc2c(sc(NC(=O)CCCCC(=O)OCCCCCCCCCCC)c2C(=O)NCCN(C)C)C1]

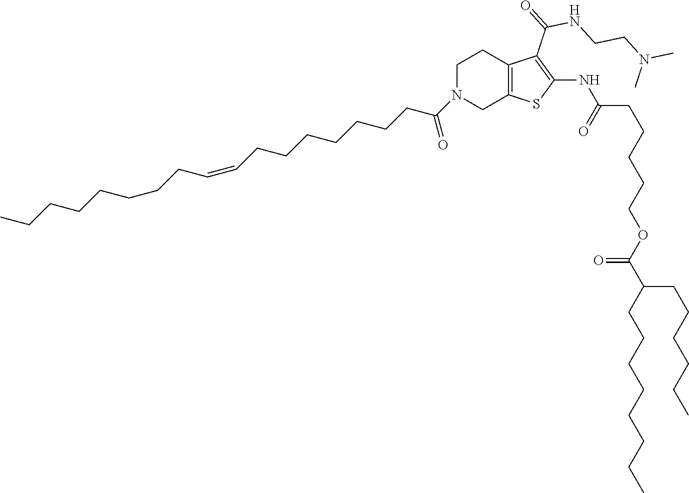 [SMILES: CCCCCCCC/C=C\CCCCCCCC(=O)N1CCc2c(sc(NC(=O)CCCCCOC(=O)C(CCCCCC)CCCCCCCC)c2C(=O)NCCN(C)C)C1]

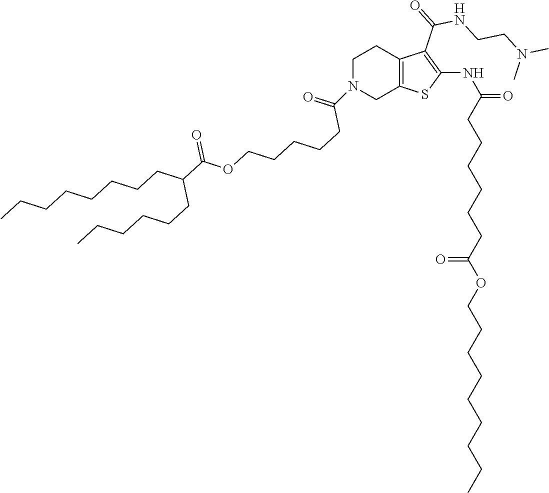 [SMILES: CCCCCCCCCOC(=O)CCCCCCC(=O)Nc1sc2c(c1C(=O)NCCN(C)C)CCN(C(=O)CCCCCOC(=O)C(CCCCCC)CCCCCCCC)C2]

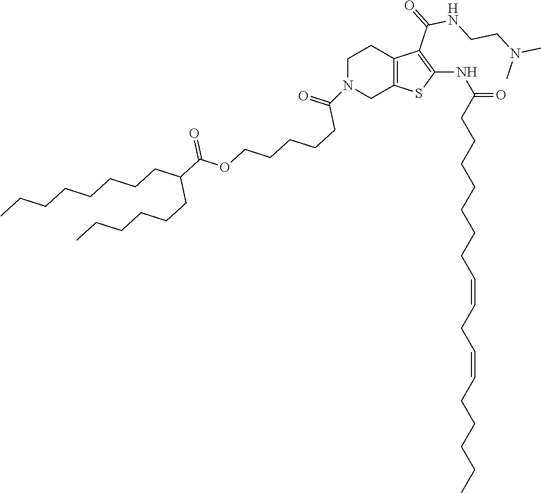 [SMILES: CCCCC/C=C\C/C=C\CCCCCCCC(=O)Nc1sc2c(c1C(=O)NCCN(C)C)CCN(C(=O)CCCCCOC(=O)C(CCCCCC)CCCCCCCC)C2]

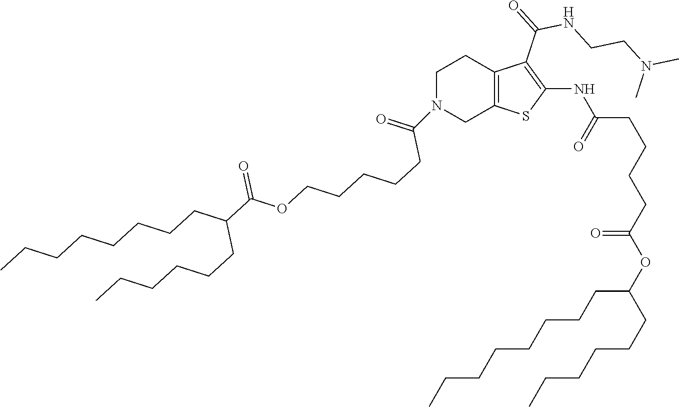 [SMILES: CCCCCCCCC(CCCCCC)OC(=O)CCCCC(=O)Nc1sc2c(c1C(=O)NCCN(C)C)CCN(C(=O)CCCCCOC(=O)C(CCCCCC)CCCCCCCC)C2]

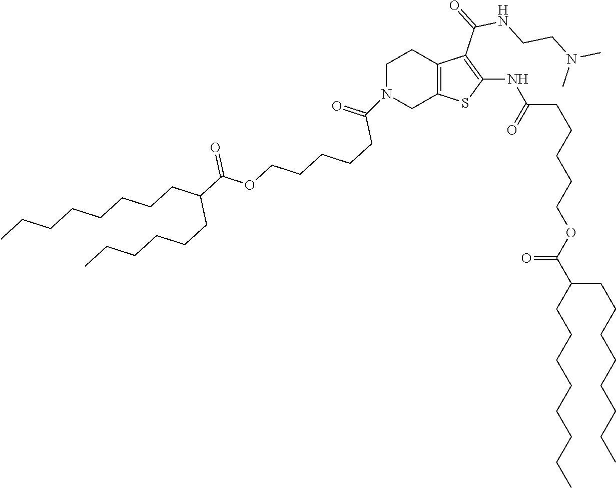 [SMILES: CCCCCCCCC(CCCCCC)C(=O)OCCCCCC(=O)N1CCc2c(sc(NC(=O)CCCCCOC(=O)C(CCCCCCCC)CCCCCCCC)c2C(=O)NCCN(C)C)C1]

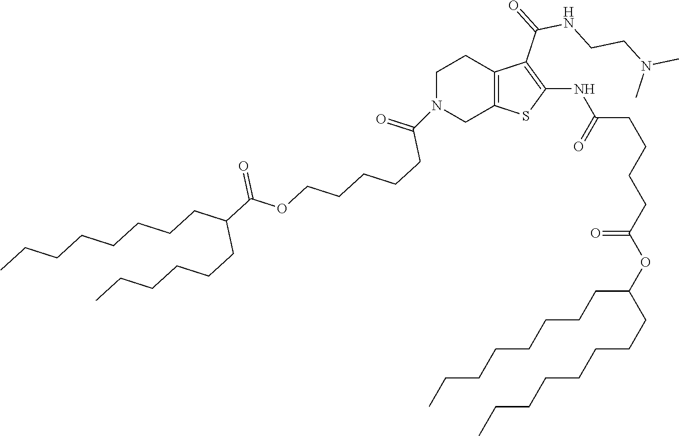 [SMILES: CCCCCCCCC(CCCCCCCC)OC(=O)CCCCC(=O)Nc1sc2c(c1C(=O)NCCN(C)C)CCN(C(=O)CCCCCOC(=O)C(CCCCCC)CCCCCCCC)C2]

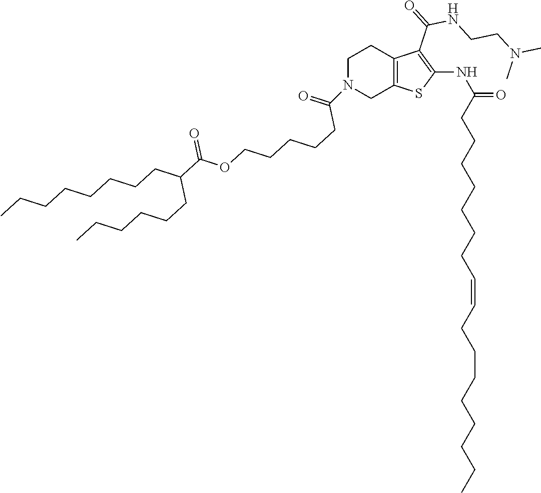 [SMILES: CCCCCCCC/C=C\CCCCCCCC(=O)Nc1sc2c(c1C(=O)NCCN(C)C)CCN(C(=O)CCCCCOC(=O)C(CCCCCC)CCCCCCCC)C2]

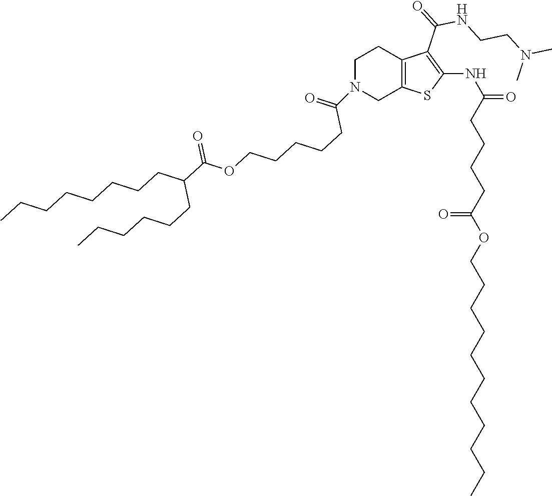 [SMILES: CCCCCCCCCCCOC(=O)CCCCC(=O)Nc1sc2c(c1C(=O)NCCN(C)C)CCN(C(=O)CCCCCOC(=O)C(CCCCCC)CCCCCCCC)C2]

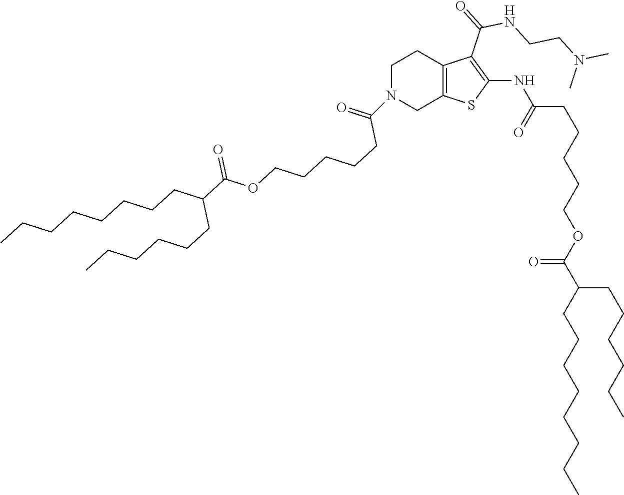 [SMILES: CCCCCCCCC(CCCCCC)C(=O)OCCCCCC(=O)Nc1sc2c(c1C(=O)NCCN(C)C)CCN(C(=O)CCCCCOC(=O)C(CCCCCC)CCCCCCCC)C2]

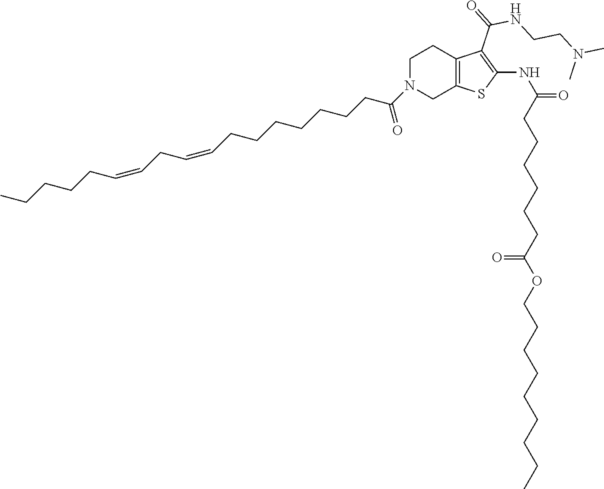 [SMILES: CCCCC/C=C\C/C=C\CCCCCCCC(=O)N1CCc2c(sc(NC(=O)CCCCCCC(=O)OCCCCCCCCC)c2C(=O)NCCN(C)C)C1]

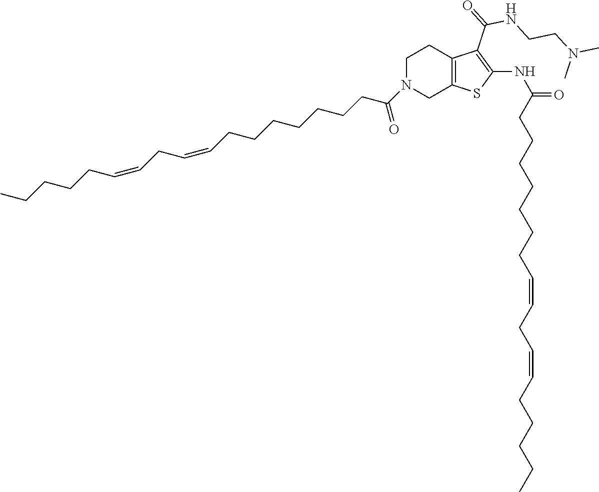 [SMILES: CCCCC/C=C\C/C=C\CCCCCCCC(=O)Nc1sc2c(c1C(=O)NCCN(C)C)CCN(C(=O)CCCCCCC/C=C\C/C=C\CCCCC)C2]

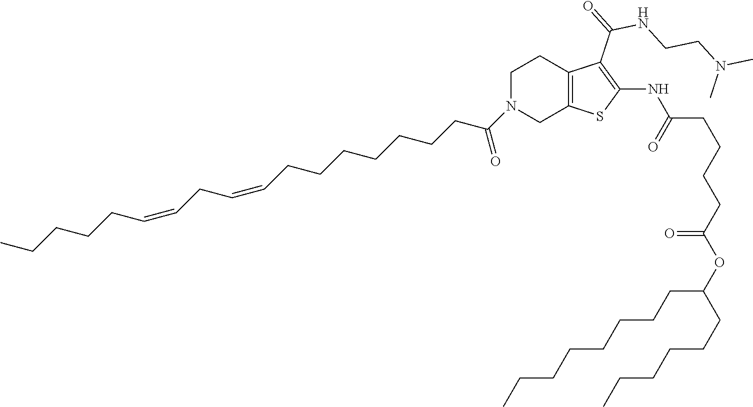 [SMILES: CCCCC/C=C\C/C=C\CCCCCCCC(=O)N1CCc2c(sc(NC(=O)CCCCC(=O)OC(CCCCCC)CCCCCCCC)c2C(=O)NCCN(C)C)C1]

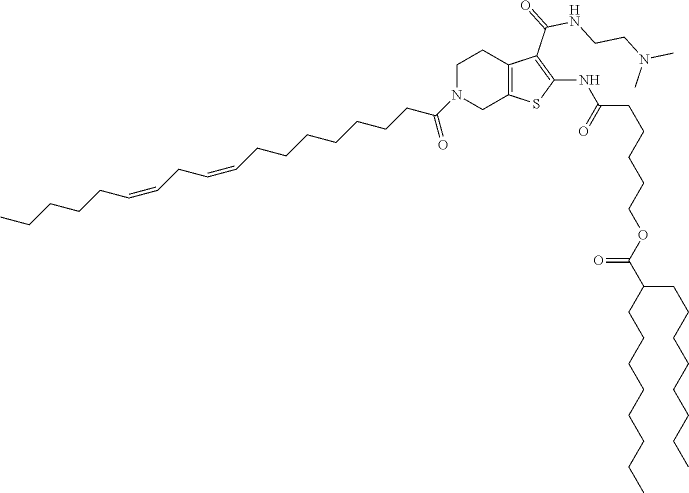 [SMILES: CCCCC/C=C\C/C=C\CCCCCCCC(=O)N1CCc2c(sc(NC(=O)CCCCCOC(=O)C(CCCCCCCC)CCCCCCCC)c2C(=O)NCCN(C)C)C1]

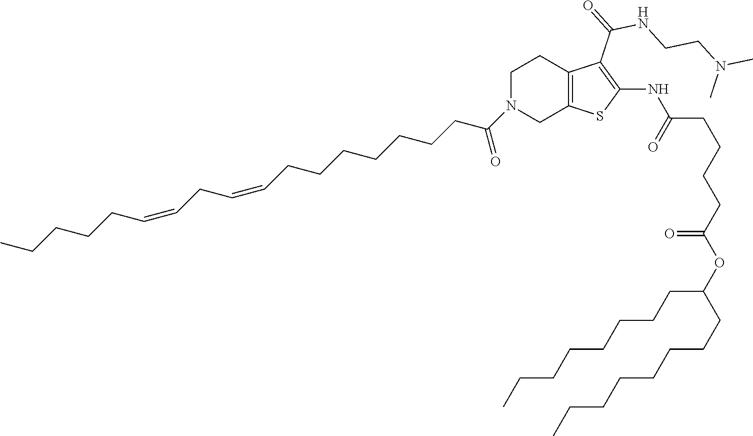 [SMILES: CCCCC/C=C\C/C=C\CCCCCCCC(=O)N1CCc2c(sc(NC(=O)CCCCC(=O)OC(CCCCCCCC)CCCCCCCC)c2C(=O)NCCN(C)C)C1]

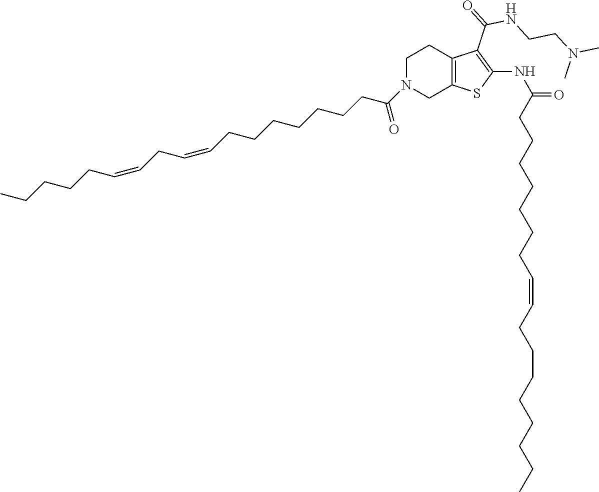 [SMILES: CCCCC/C=C\C/C=C\CCCCCCCC(=O)N1CCc2c(sc(NC(=O)CCCCCCC/C=C\CCCCCCCC)c2C(=O)NCCN(C)C)C1]

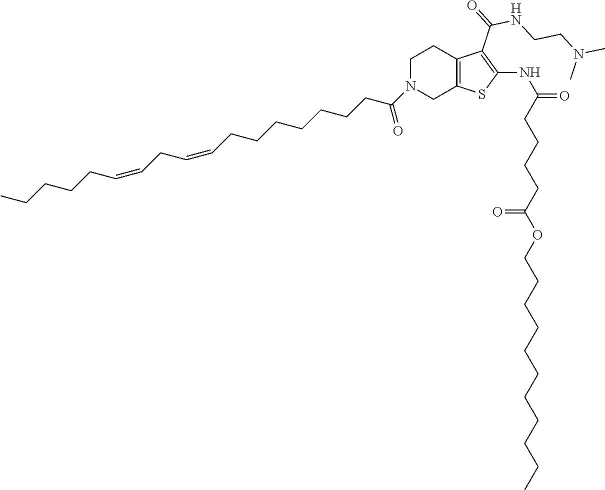 [SMILES: CCCCC/C=C\C/C=C\CCCCCCCC(=O)N1CCc2c(sc(NC(=O)CCCCC(=O)OCCCCCCCCCCC)c2C(=O)NCCN(C)C)C1]

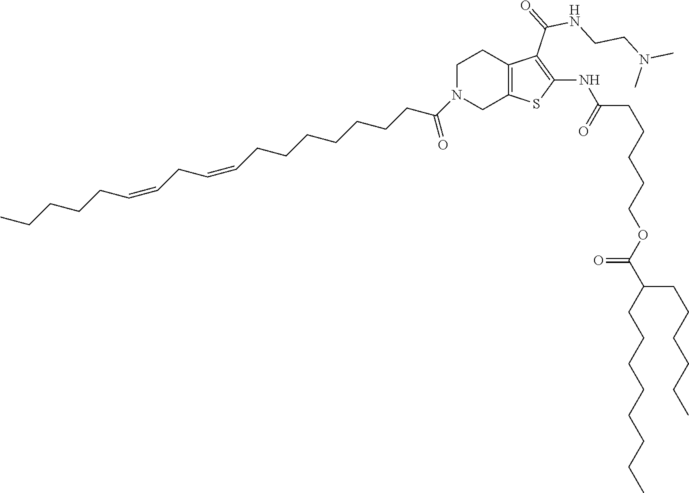 [SMILES: CCCCC/C=C\C/C=C\CCCCCCCC(=O)N1CCc2c(sc(NC(=O)CCCCCOC(=O)C(CCCCCC)CCCCCCCC)c2C(=O)NCCN(C)C)C1]

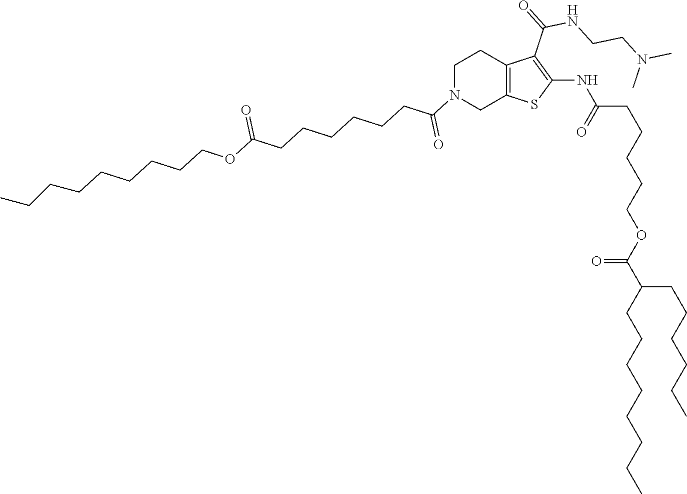 [SMILES: CCCCCCCCCOC(=O)CCCCCCC(=O)N1CCc2c(sc(NC(=O)CCCCCOC(=O)C(CCCCCC)CCCCCCCC)c2C(=O)NCCN(C)C)C1]

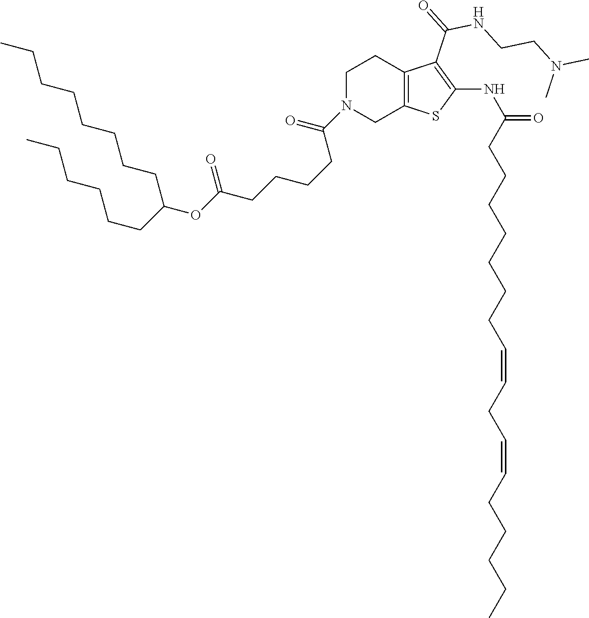 [SMILES: CCCCC/C=C\C/C=C\CCCCCCCC(=O)Nc1sc2c(c1C(=O)NCCN(C)C)CCN(C(=O)CCCCC(=O)OC(CCCCCC)CCCCCCCC)C2]

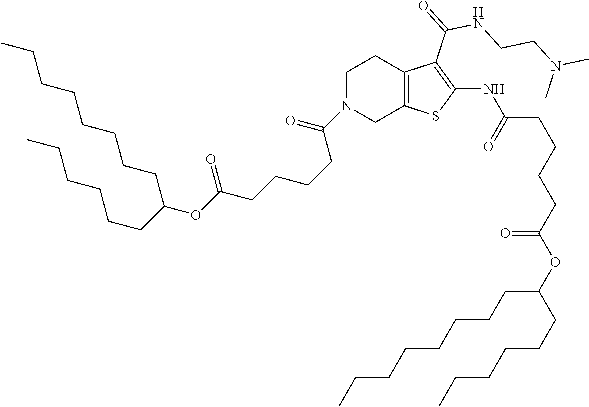 [SMILES: CCCCCCCCC(CCCCCC)OC(=O)CCCCC(=O)Nc1sc2c(c1C(=O)NCCN(C)C)CCN(C(=O)CCCCC(=O)OC(CCCCCC)CCCCCCCC)C2]

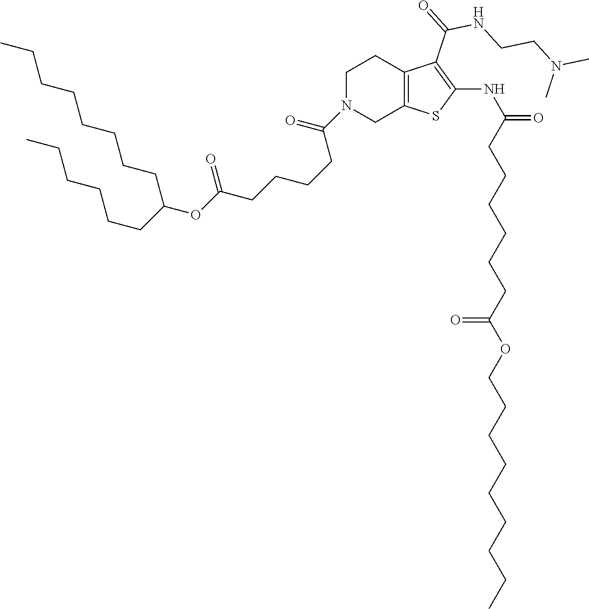 [SMILES: CCCCCCCCCOC(=O)CCCCCCC(=O)Nc1sc2c(c1C(=O)NCCN(C)C)CCN(C(=O)CCCCC(=O)OC(CCCCCC)CCCCCCCC)C2]

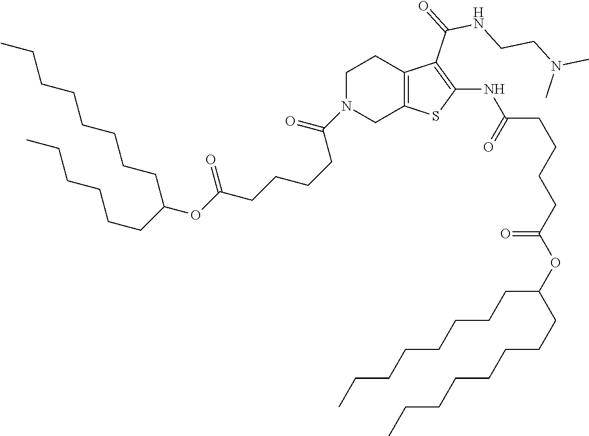 [SMILES: CCCCCCCCC(CCCCCCCC)OC(=O)CCCCC(=O)Nc1sc2c(c1C(=O)NCCN(C)C)CCN(C(=O)CCCCC(=O)OC(CCCCCC)CCCCCCCC)C2]

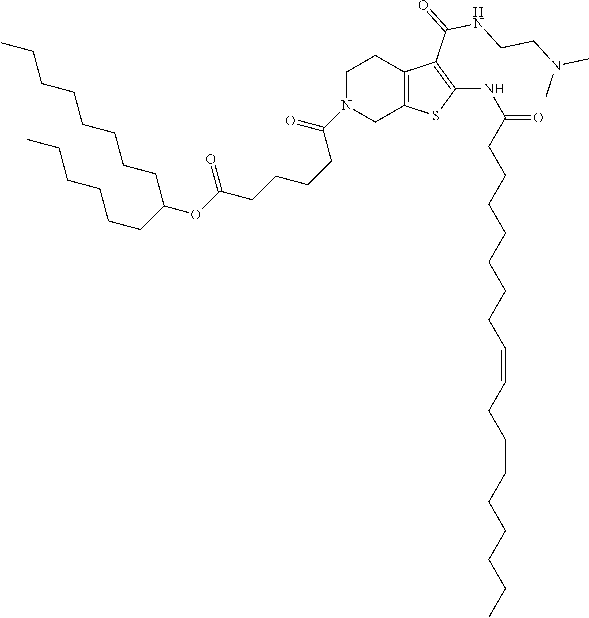 [SMILES: CCCCCCCC/C=C\CCCCCCCC(=O)Nc1sc2c(c1C(=O)NCCN(C)C)CCN(C(=O)CCCCC(=O)OC(CCCCCC)CCCCCCCC)C2]

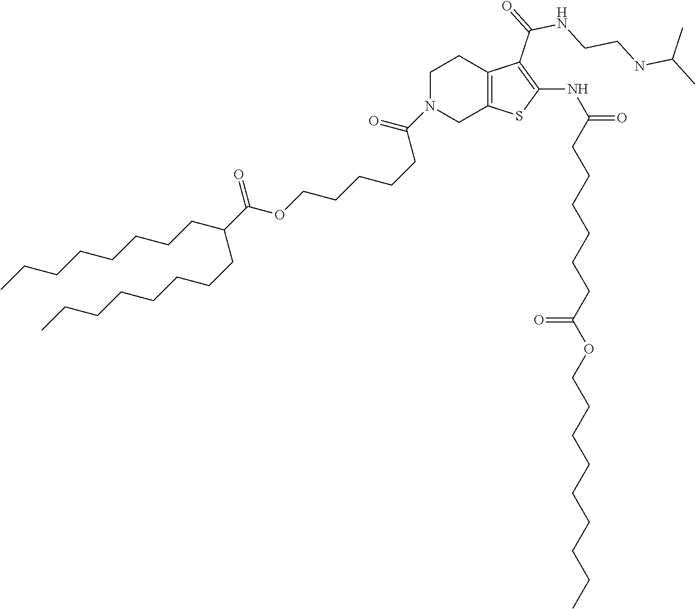 [SMILES: CCCCCCCCCOC(=O)CCCCCCC(=O)Nc1sc2c(c1C(=O)NCCNC(C)C)CCN(C(=O)CCCCCOC(=O)C(CCCCCCCC)CCCCCCCC)C2]

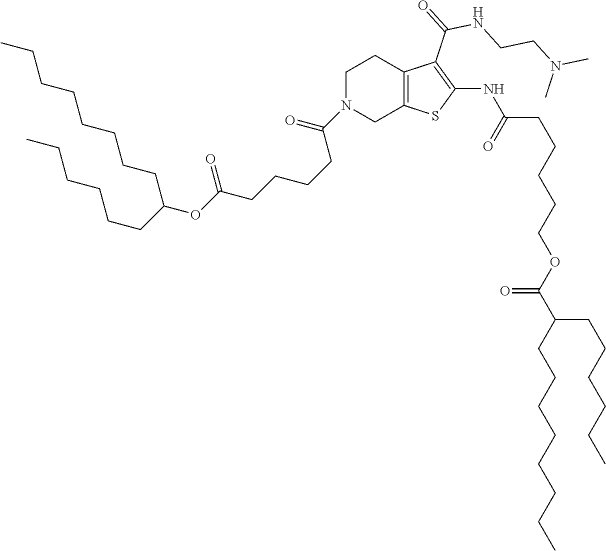 [SMILES: CCCCCCCCC(CCCCCC)OC(=O)CCCCC(=O)N1CCc2c(sc(NC(=O)CCCCCOC(=O)C(CCCCCC)CCCCCCCC)c2C(=O)NCCN(C)C)C1]

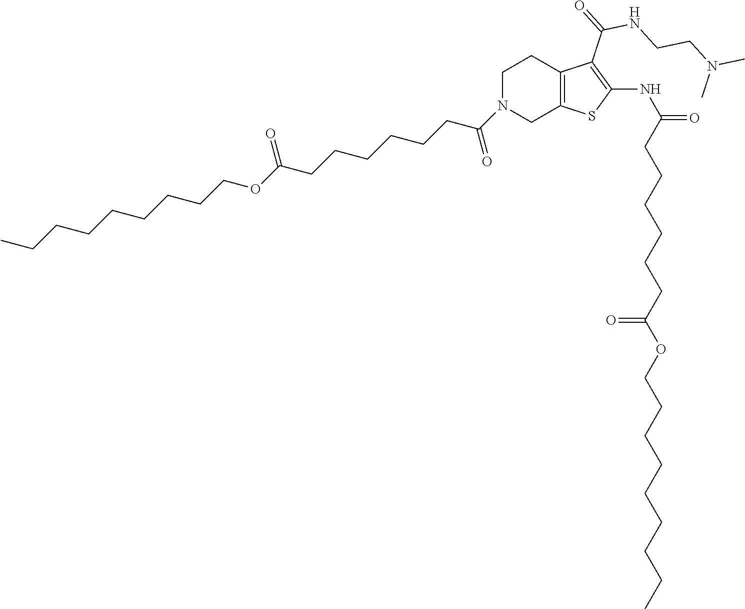 [SMILES: CCCCCCCCCOC(=O)CCCCCCC(=O)Nc1sc2c(c1C(=O)NCCN(C)C)CCN(C(=O)CCCCCCC(=O)OCCCCCCCCC)C2]

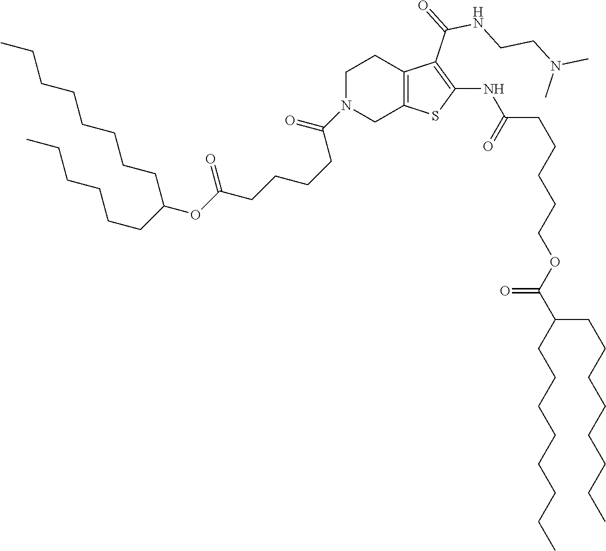 [SMILES: CCCCCCCCC(CCCCCC)OC(=O)CCCCC(=O)N1CCc2c(sc(NC(=O)CCCCCOC(=O)C(CCCCCCCC)CCCCCCCC)c2C(=O)NCCN(C)C)C1]

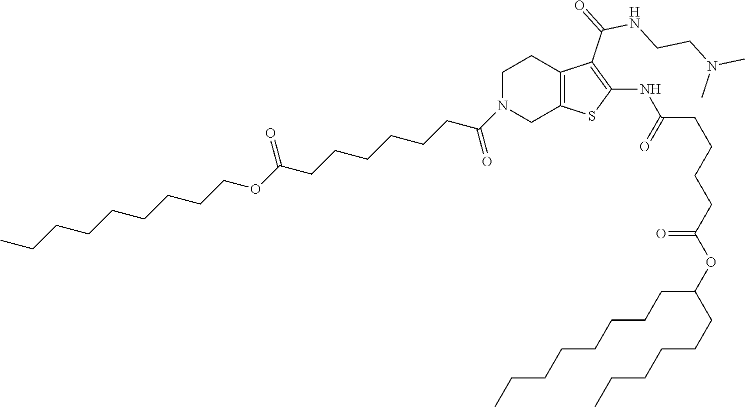 [SMILES: CCCCCCCCCOC(=O)CCCCCCC(=O)N1CCc2c(sc(NC(=O)CCCCC(=O)OC(CCCCCC)CCCCCCCC)c2C(=O)NCCN(C)C)C1]

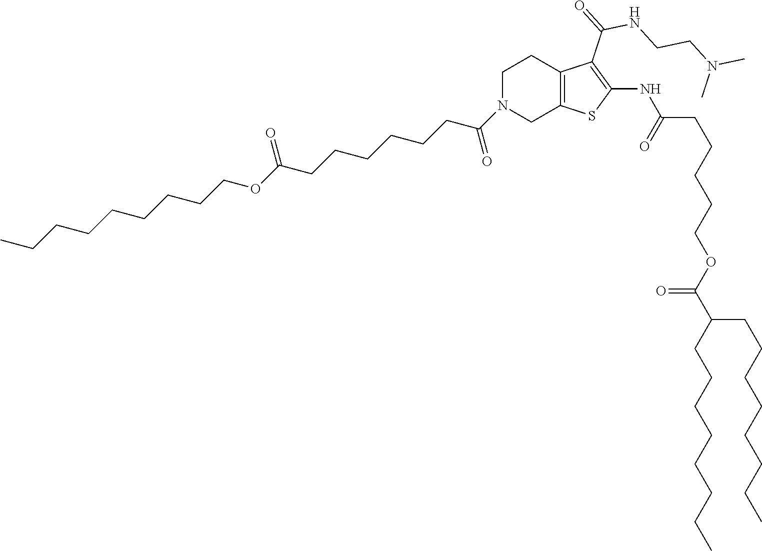 [SMILES: CCCCCCCCCOC(=O)CCCCCCC(=O)N1CCc2c(sc(NC(=O)CCCCCOC(=O)C(CCCCCCCC)CCCCCCCC)c2C(=O)NCCN(C)C)C1]

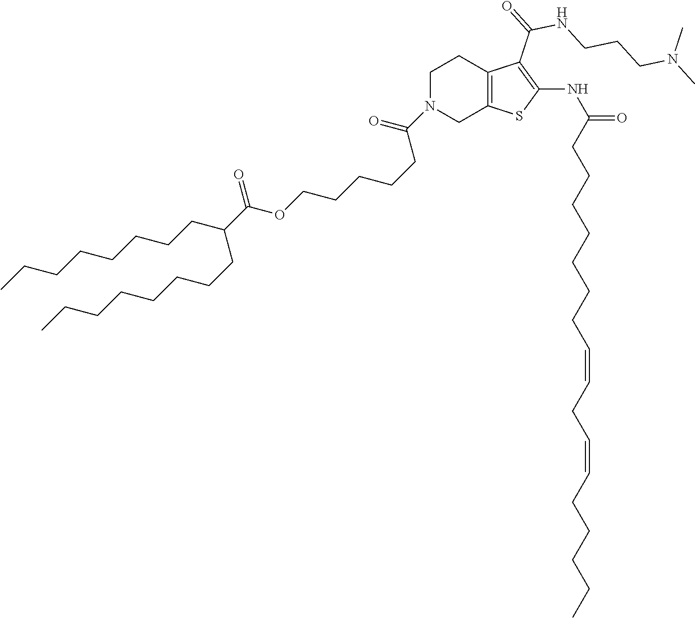 [SMILES: CCCCC/C=C\C/C=C\CCCCCCCC(=O)Nc1sc2c(c1C(=O)NCCCN(C)C)CCN(C(=O)CCCCCOC(=O)C(CCCCCCCC)CCCCCCCC)C2]

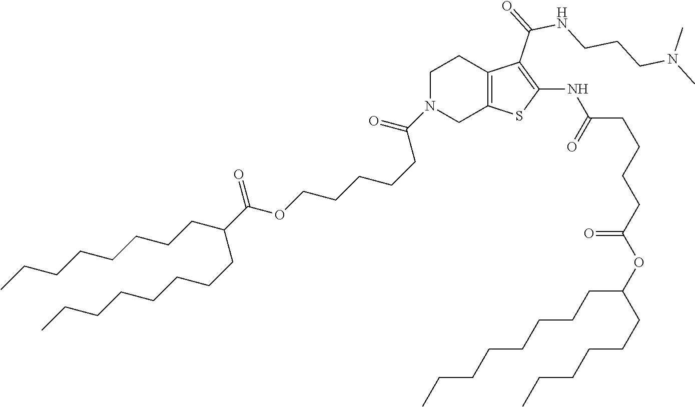 [SMILES: CCCCCCCCC(CCCCCC)OC(=O)CCCCC(=O)Nc1sc2c(c1C(=O)NCCCN(C)C)CCN(C(=O)CCCCCOC(=O)C(CCCCCCCC)CCCCCCCC)C2]

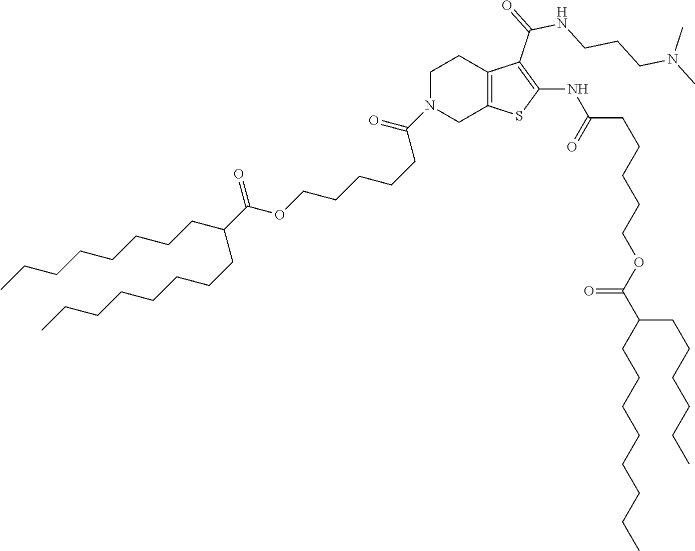 [SMILES: CCCCCCCCC(CCCCCC)C(=O)OCCCCCC(=O)Nc1sc2c(c1C(=O)NCCCN(C)C)CCN(C(=O)CCCCCOC(=O)C(CCCCCCCC)CCCCCCCC)C2]

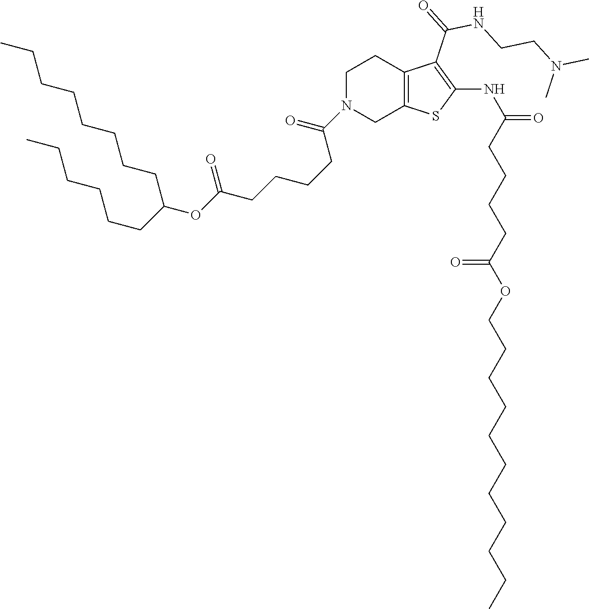 [SMILES: CCCCCCCCCCCOC(=O)CCCCC(=O)Nc1sc2c(c1C(=O)NCCN(C)C)CCN(C(=O)CCCCC(=O)OC(CCCCCC)CCCCCCCC)C2]

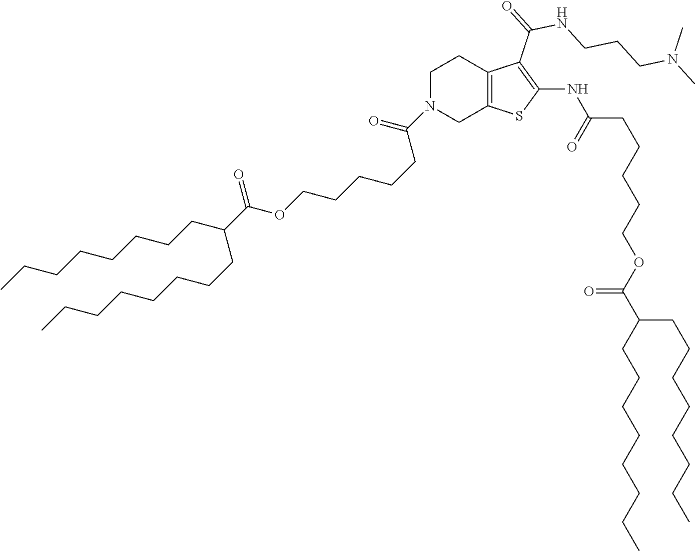 [SMILES: CCCCCCCCC(CCCCCCCC)C(=O)OCCCCCC(=O)Nc1sc2c(c1C(=O)NCCCN(C)C)CCN(C(=O)CCCCCOC(=O)C(CCCCCCCC)CCCCCCCC)C2]

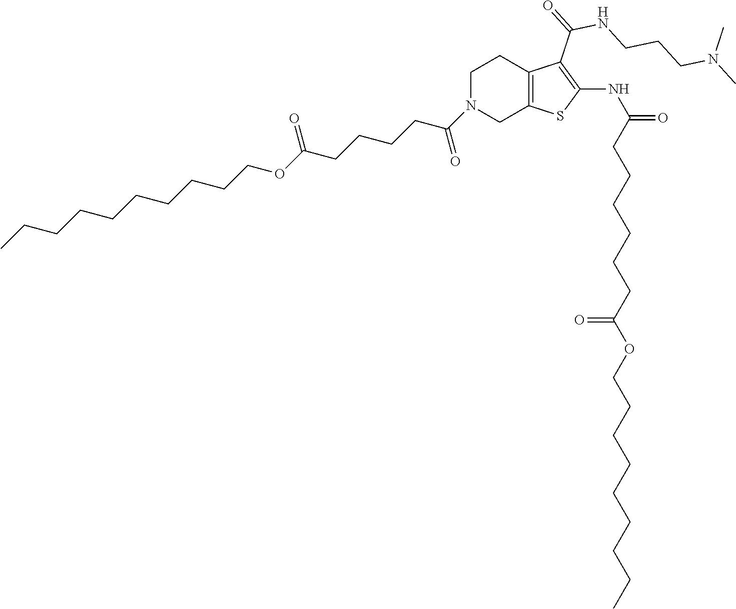 [SMILES: CCCCCCCCCCOC(=O)CCCCC(=O)N1CCc2c(sc(NC(=O)CCCCCCC(=O)OCCCCCCCCC)c2C(=O)NCCCN(C)C)C1]

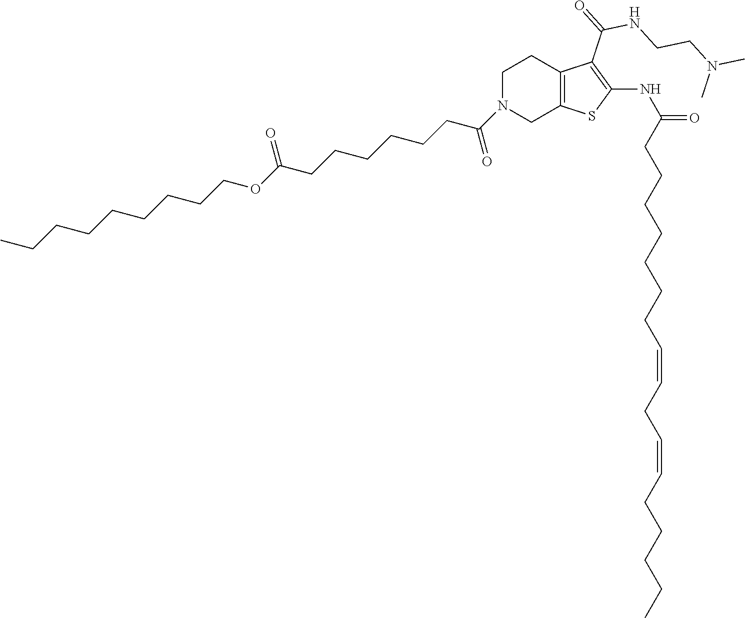 [SMILES: CCCCC/C=C\C/C=C\CCCCCCCC(=O)Nc1sc2c(c1C(=O)NCCN(C)C)CCN(C(=O)CCCCCCC(=O)OCCCCCCCCC)C2]

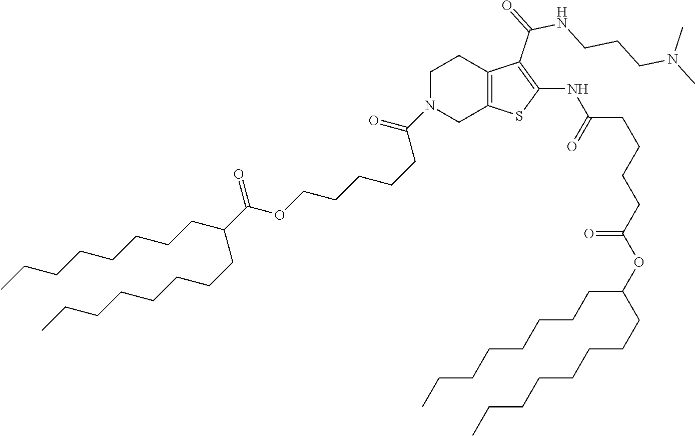 [SMILES: CCCCCCCCC(CCCCCCCC)OC(=O)CCCCC(=O)Nc1sc2c(c1C(=O)NCCCN(C)C)CCN(C(=O)CCCCCOC(=O)C(CCCCCCCC)CCCCCCCC)C2]

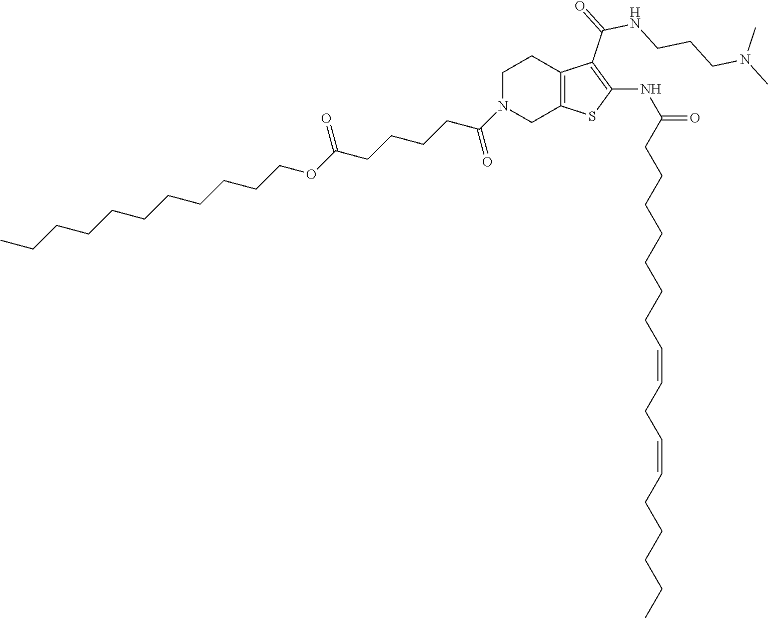 [SMILES: CCCCC/C=C\C/C=C\CCCCCCCC(=O)Nc1sc2c(c1C(=O)NCCCN(C)C)CCN(C(=O)CCCCC(=O)OCCCCCCCCCCC)C2]

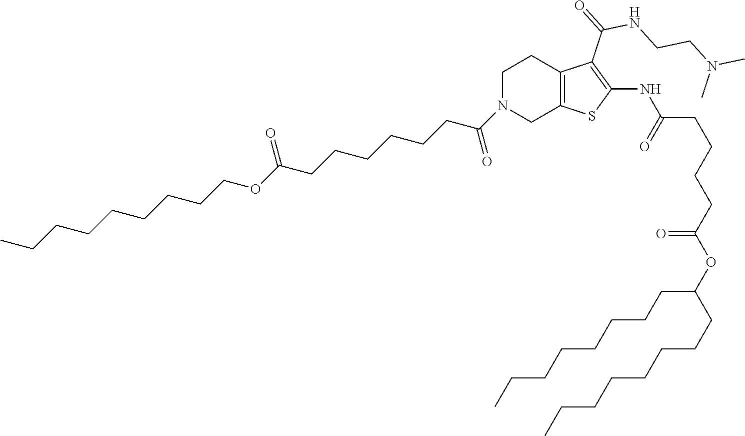 [SMILES: CCCCCCCCCOC(=O)CCCCCCC(=O)N1CCc2c(sc(NC(=O)CCCCC(=O)OC(CCCCCCCC)CCCCCCCC)c2C(=O)NCCN(C)C)C1]

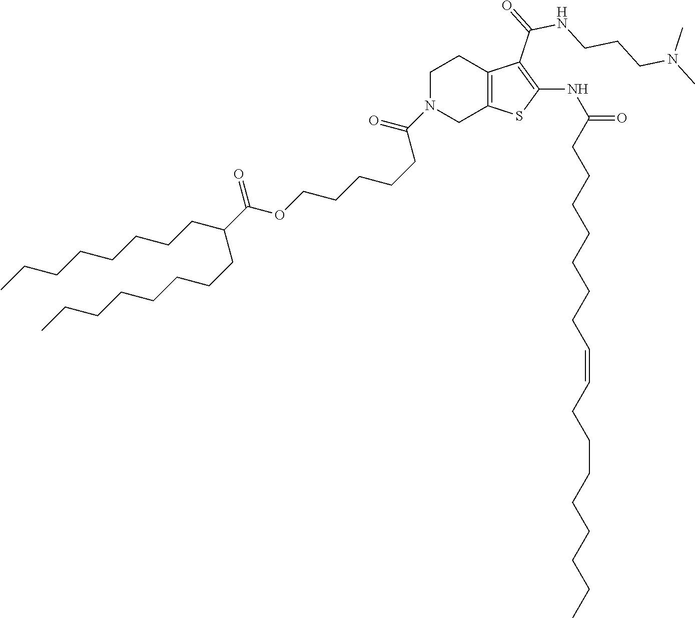 [SMILES: CCCCCCCC/C=C\CCCCCCCC(=O)Nc1sc2c(c1C(=O)NCCCN(C)C)CCN(C(=O)CCCCCOC(=O)C(CCCCCCCC)CCCCCCCC)C2]

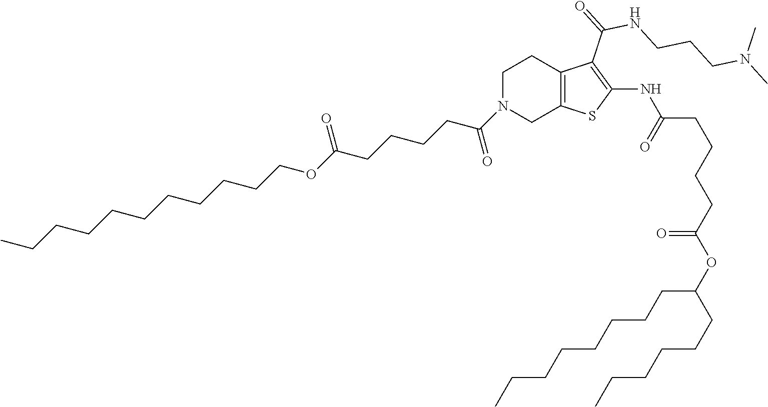 [SMILES: CCCCCCCCCCCOC(=O)CCCCC(=O)N1CCc2c(sc(NC(=O)CCCCC(=O)OC(CCCCCC)CCCCCCCC)c2C(=O)NCCCN(C)C)C1]

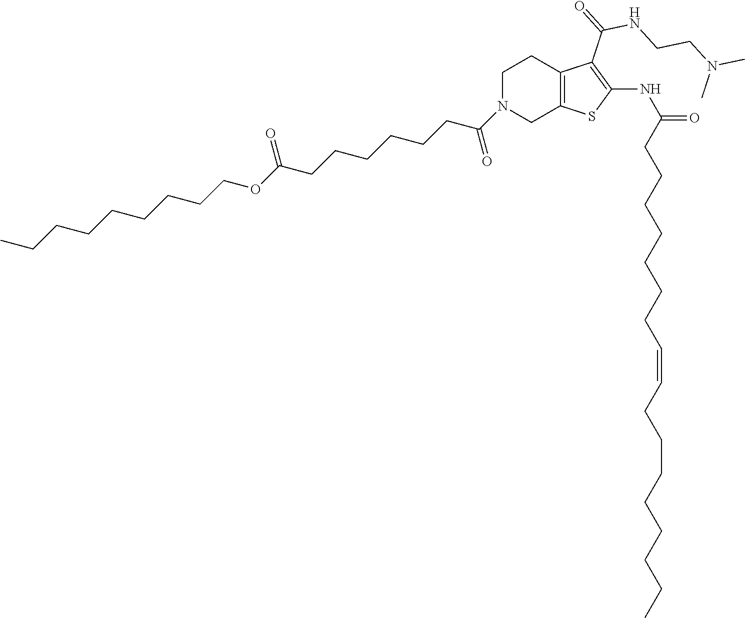 [SMILES: CCCCCCCC/C=C\CCCCCCCC(=O)Nc1sc2c(c1C(=O)NCCN(C)C)CCN(C(=O)CCCCCCC(=O)OCCCCCCCCC)C2]

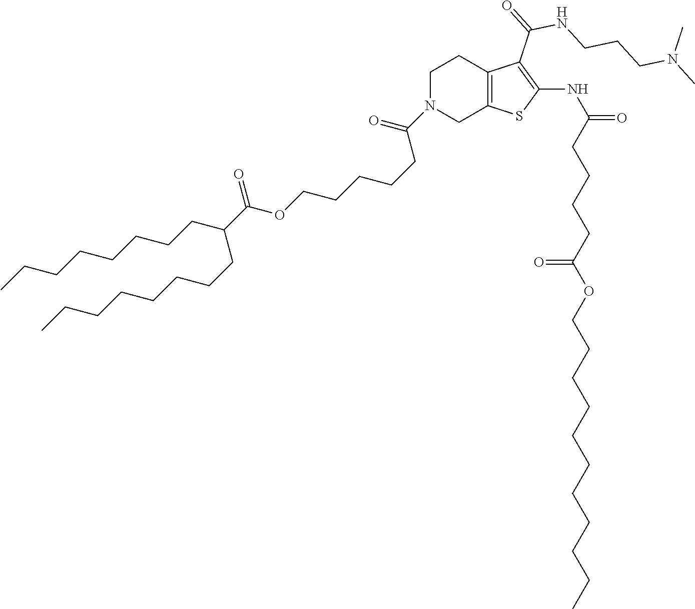 [SMILES: CCCCCCCCCCCOC(=O)CCCCC(=O)Nc1sc2c(c1C(=O)NCCCN(C)C)CCN(C(=O)CCCCCOC(=O)C(CCCCCCCC)CCCCCCCC)C2]

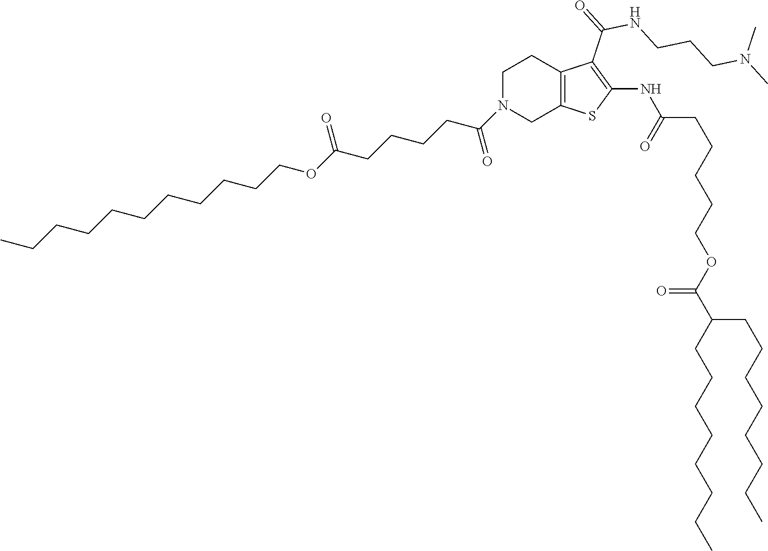 [SMILES: CCCCCCCCCCCOC(=O)CCCCC(=O)N1CCc2c(sc(NC(=O)CCCCCOC(=O)C(CCCCCCCC)CCCCCCCC)c2C(=O)NCCCN(C)C)C1]

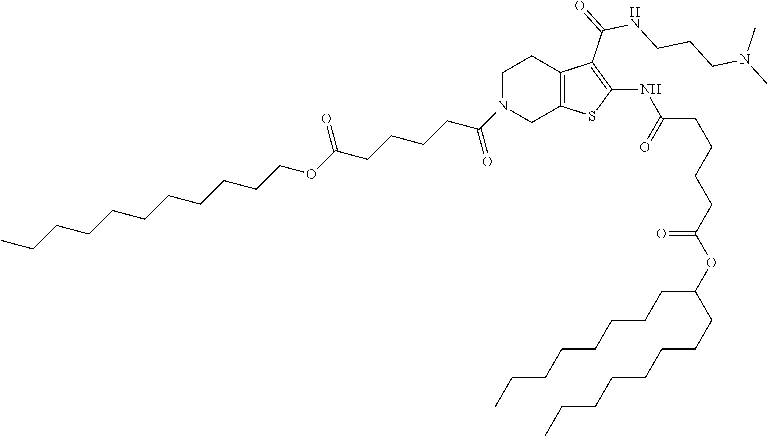 [SMILES: CCCCCCCCCCCOC(=O)CCCCC(=O)N1CCc2c(sc(NC(=O)CCCCC(=O)OC(CCCCCCCC)CCCCCCCC)c2C(=O)NCCCN(C)C)C1]

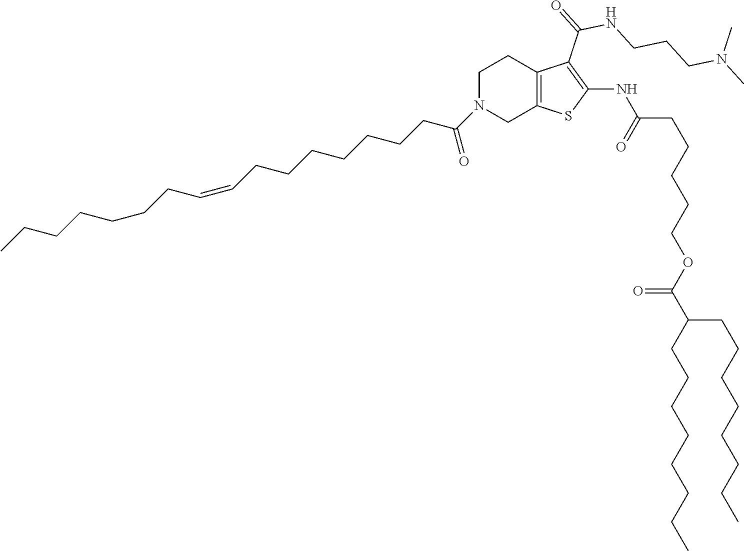 [SMILES: CCCCCCC/C=C\CCCCCCCC(=O)N1CCc2c(sc(NC(=O)CCCCCOC(=O)C(CCCCCCCC)CCCCCCCC)c2C(=O)NCCCN(C)C)C1]

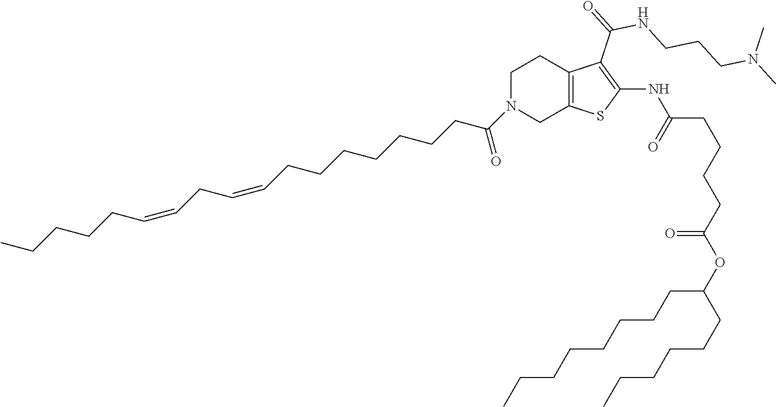 [SMILES: CCCCC/C=C\C/C=C\CCCCCCCC(=O)N1CCc2c(sc(NC(=O)CCCCC(=O)OC(CCCCCC)CCCCCCCC)c2C(=O)NCCCN(C)C)C1]

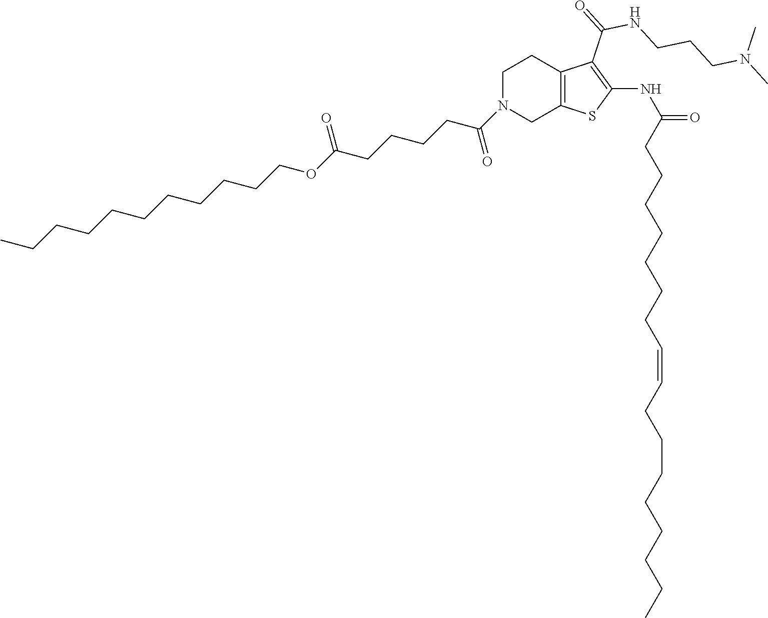 [SMILES: CCCCCCCC/C=C\CCCCCCCC(=O)Nc1sc2c(c1C(=O)NCCCN(C)C)CCN(C(=O)CCCCC(=O)OCCCCCCCCCCC)C2]

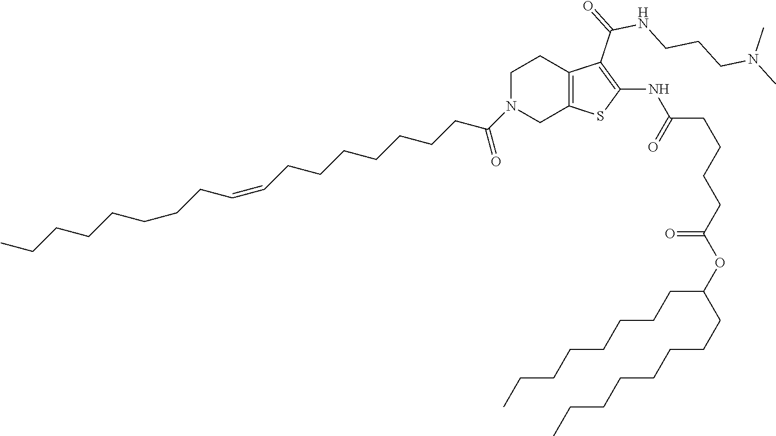 [SMILES: CCCCCCCC/C=C\CCCCCCCC(=O)N1CCc2c(sc(NC(=O)CCCCC(=O)OC(CCCCCCCC)CCCCCCCC)c2C(=O)NCCCN(C)C)C1]

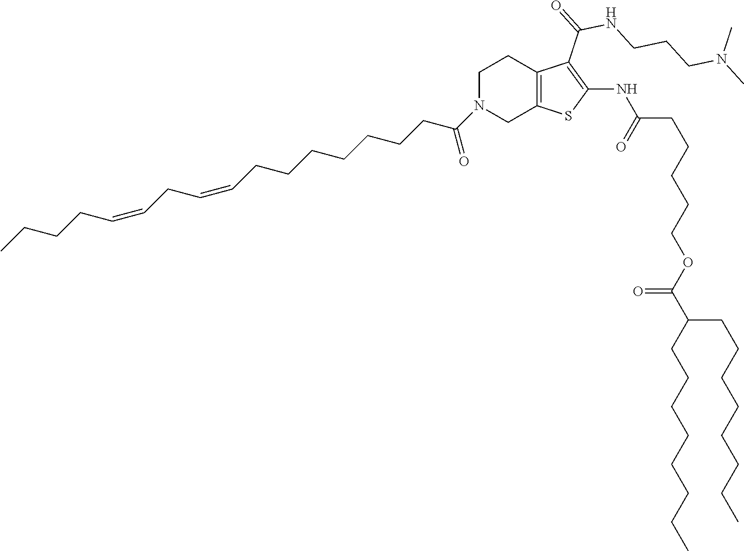 [SMILES: CCCC/C=C\C/C=C\CCCCCCCC(=O)N1CCc2c(sc(NC(=O)CCCCCOC(=O)C(CCCCCCCC)CCCCCCCC)c2C(=O)NCCCN(C)C)C1]

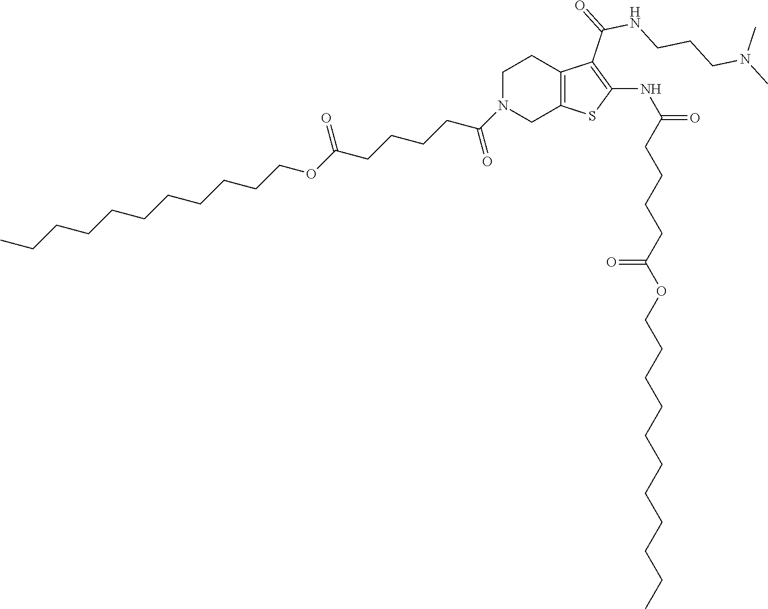 [SMILES: CCCCCCCCCCCOC(=O)CCCCC(=O)Nc1sc2c(c1C(=O)NCCCN(C)C)CCN(C(=O)CCCCC(=O)OCCCCCCCCCCC)C2]

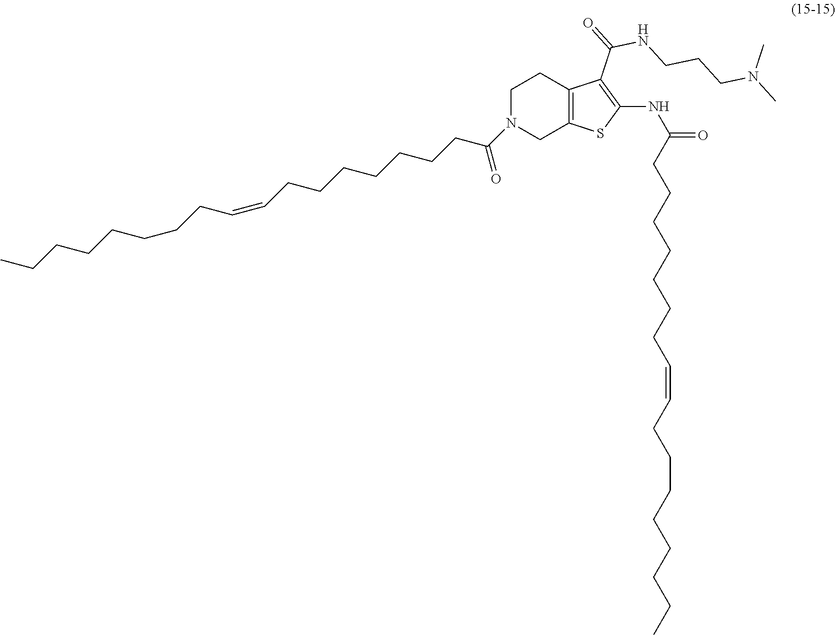 [SMILES: CCCCCCCC/C=C\CCCCCCCC(=O)Nc1sc2c(c1C(=O)NCCCN(C)C)CCN(C(=O)CCCCCCC/C=C\CCCCCCCC)C2]

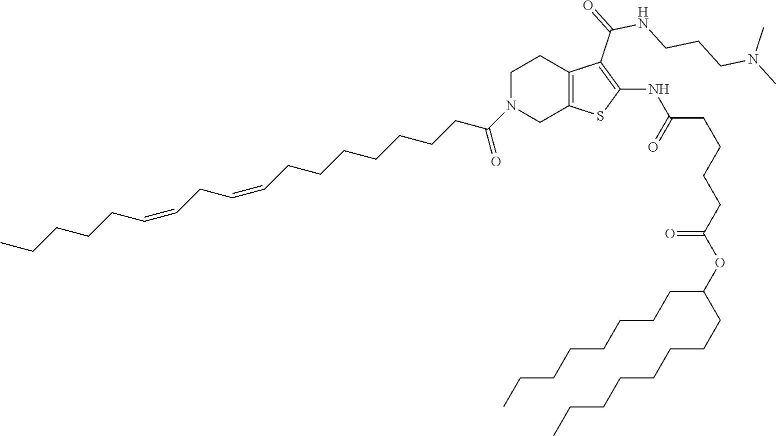 [SMILES: CCCCC/C=C\C/C=C\CCCCCCCC(=O)N1CCc2c(sc(NC(=O)CCCCC(=O)OC(CCCCCCCC)CCCCCCCC)c2C(=O)NCCCN(C)C)C1]

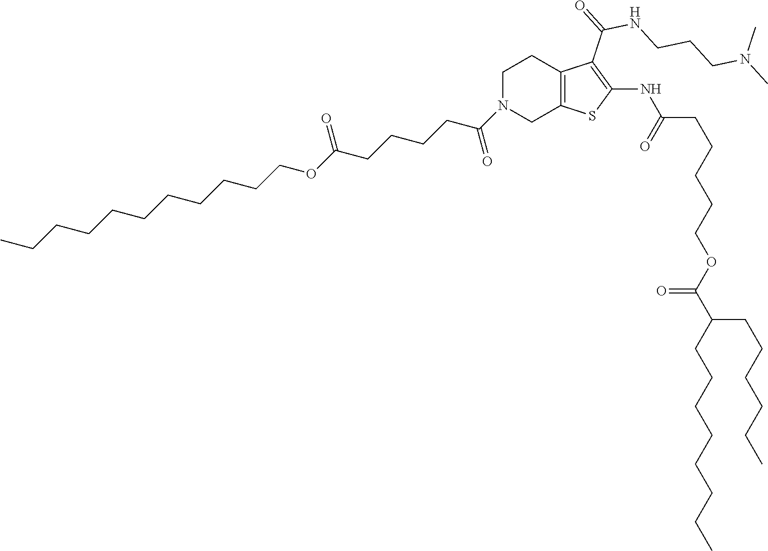 [SMILES: CCCCCCCCCCCOC(=O)CCCCC(=O)N1CCc2c(sc(NC(=O)CCCCCOC(=O)C(CCCCCC)CCCCCCCC)c2C(=O)NCCCN(C)C)C1]

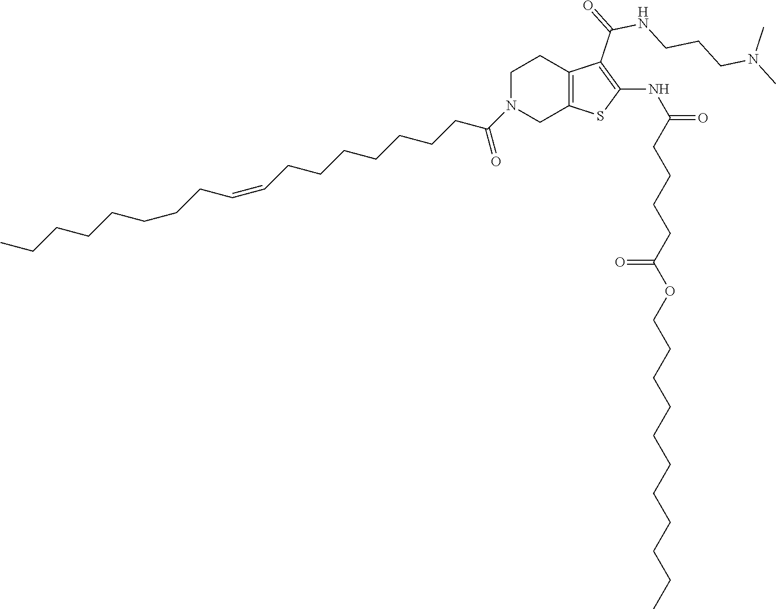 [SMILES: CCCCCCCC/C=C\CCCCCCCC(=O)N1CCc2c(sc(NC(=O)CCCCC(=O)OCCCCCCCCCCC)c2C(=O)NCCCN(C)C)C1]

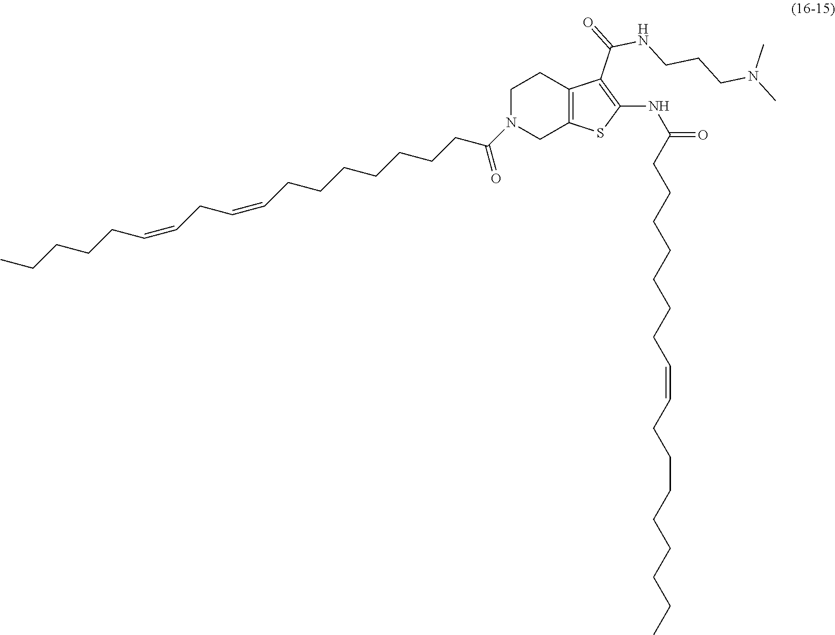 [SMILES: CCCCC/C=C\C/C=C\CCCCCCCC(=O)N1CCc2c(sc(NC(=O)CCCCCCC/C=C\CCCCCCCC)c2C(=O)NCCCN(C)C)C1]

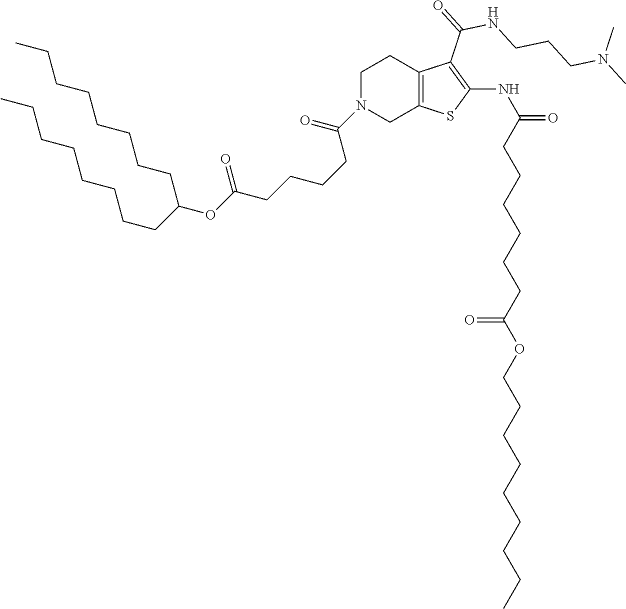 [SMILES: CCCCCCCCCOC(=O)CCCCCCC(=O)Nc1sc2c(c1C(=O)NCCCN(C)C)CCN(C(=O)CCCCC(=O)OC(CCCCCCCC)CCCCCCCC)C2]

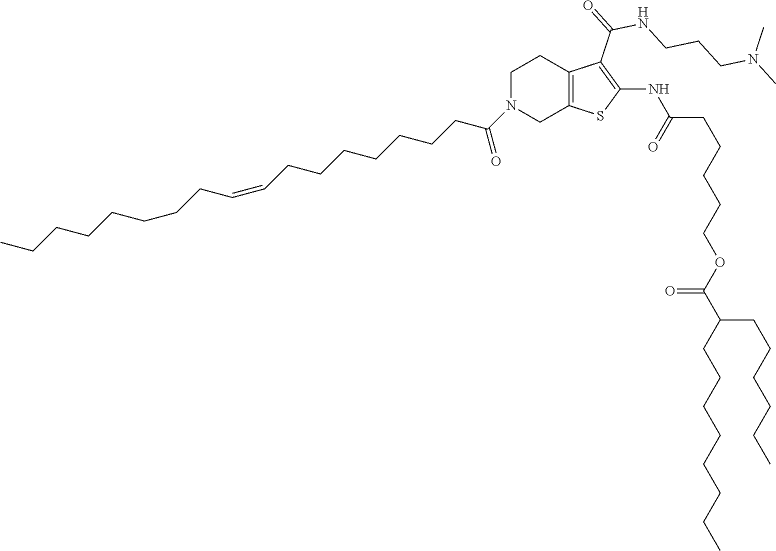 [SMILES: CCCCCCCC/C=C\CCCCCCCC(=O)N1CCc2c(sc(NC(=O)CCCCCOC(=O)C(CCCCCC)CCCCCCCC)c2C(=O)NCCCN(C)C)C1]

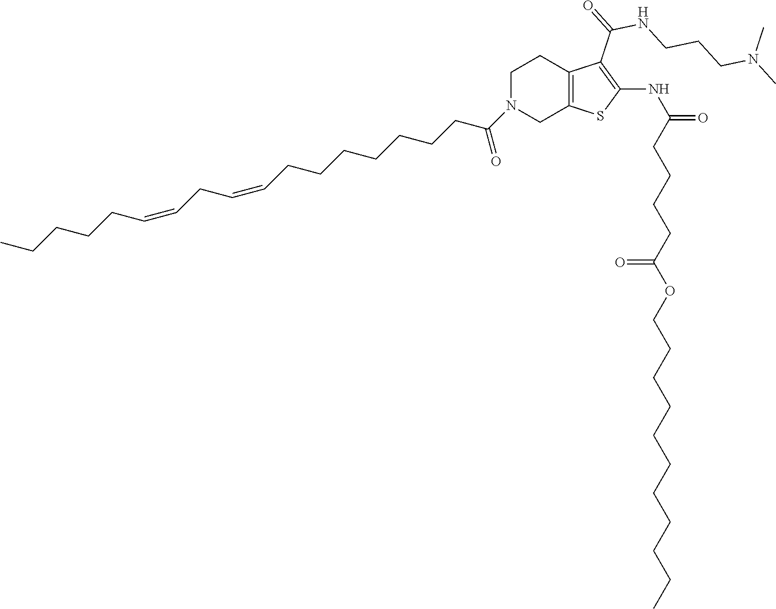 [SMILES: CCCCC/C=C\C/C=C\CCCCCCCC(=O)N1CCc2c(sc(NC(=O)CCCCC(=O)OCCCCCCCCCCC)c2C(=O)NCCCN(C)C)C1]

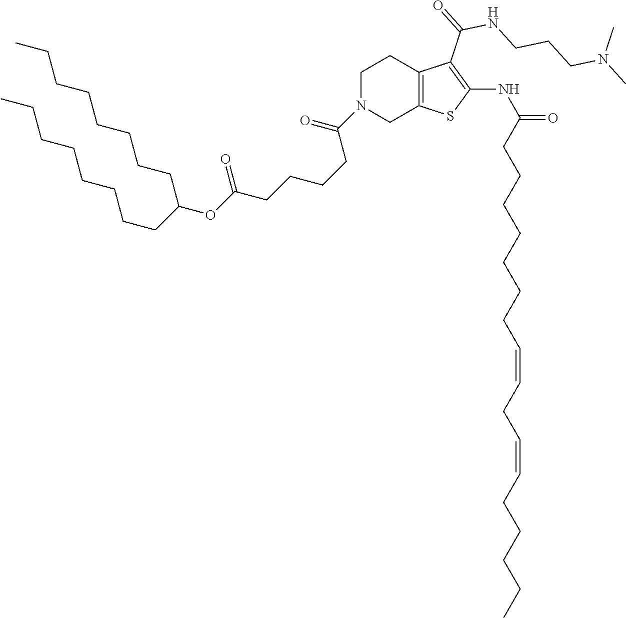 [SMILES: CCCCC/C=C\C/C=C\CCCCCCCC(=O)Nc1sc2c(c1C(=O)NCCCN(C)C)CCN(C(=O)CCCCC(=O)OC(CCCCCCCC)CCCCCCCC)C2]

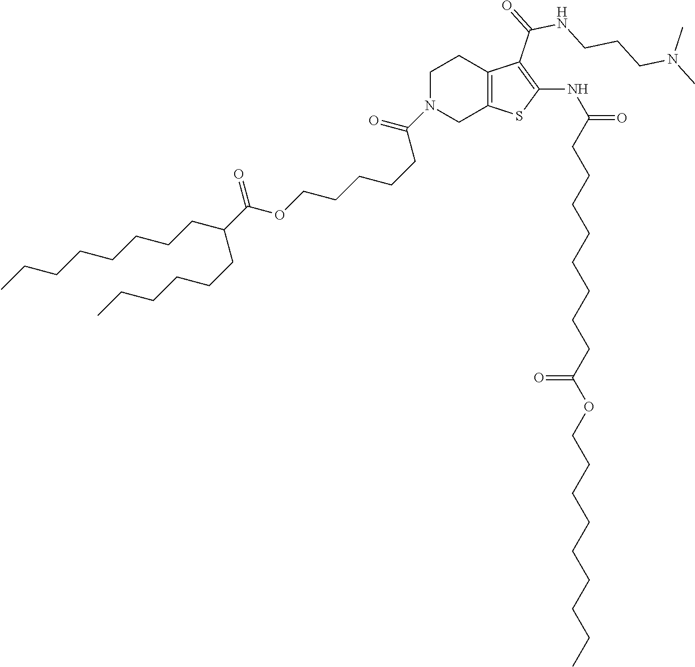 [SMILES: CCCCCCCCCOC(=O)CCCCCCCCC(=O)Nc1sc2c(c1C(=O)NCCCN(C)C)CCN(C(=O)CCCCCOC(=O)C(CCCCCC)CCCCCCCC)C2]

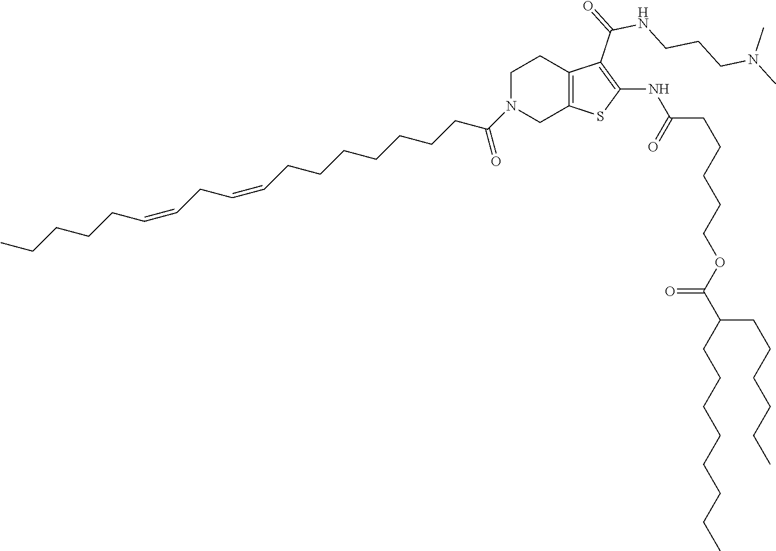 [SMILES: CCCCC/C=C\C/C=C\CCCCCCCC(=O)N1CCc2c(sc(NC(=O)CCCCCOC(=O)C(CCCCCC)CCCCCCCC)c2C(=O)NCCCN(C)C)C1]

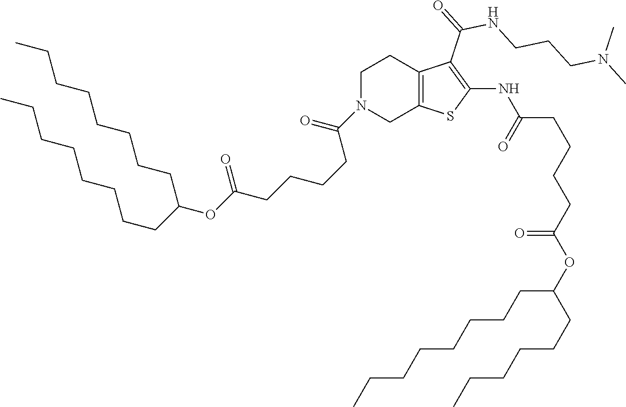 [SMILES: CCCCCCCCC(CCCCCC)OC(=O)CCCCC(=O)Nc1sc2c(c1C(=O)NCCCN(C)C)CCN(C(=O)CCCCC(=O)OC(CCCCCCCC)CCCCCCCC)C2]

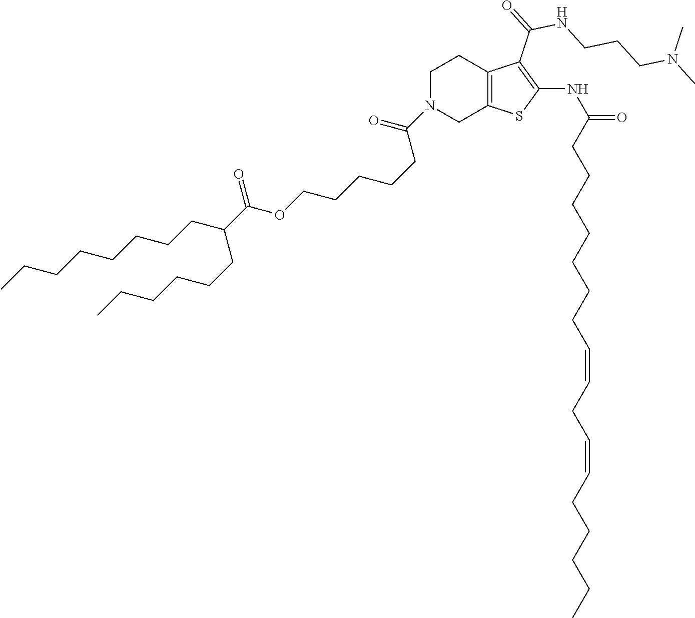 [SMILES: CCCCC/C=C\C/C=C\CCCCCCCC(=O)Nc1sc2c(c1C(=O)NCCCN(C)C)CCN(C(=O)CCCCCOC(=O)C(CCCCCC)CCCCCCCC)C2]

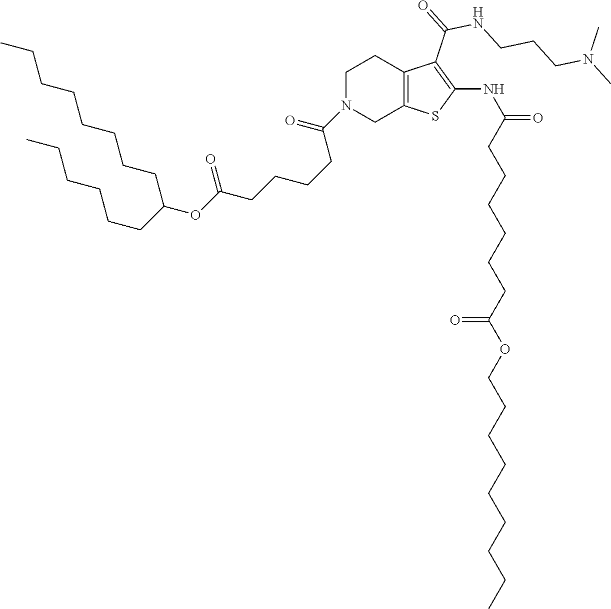 [SMILES: CCCCCCCCCOC(=O)CCCCCCC(=O)Nc1sc2c(c1C(=O)NCCCN(C)C)CCN(C(=O)CCCCC(=O)OC(CCCCCC)CCCCCCCC)C2]

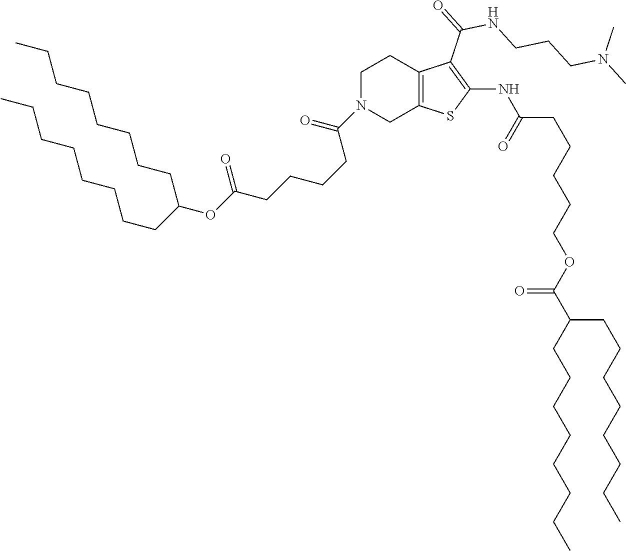 [SMILES: CCCCCCCCC(CCCCCCCC)OC(=O)CCCCC(=O)N1CCc2c(sc(NC(=O)CCCCCOC(=O)C(CCCCCCCC)CCCCCCCC)c2C(=O)NCCCN(C)C)C1]

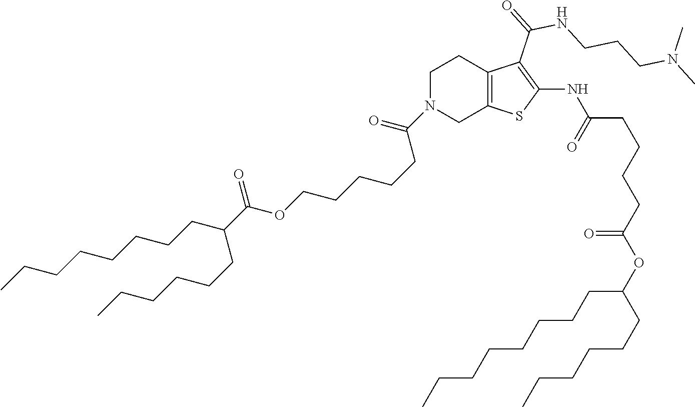 [SMILES: CCCCCCCCC(CCCCCC)OC(=O)CCCCC(=O)Nc1sc2c(c1C(=O)NCCCN(C)C)CCN(C(=O)CCCCCOC(=O)C(CCCCCC)CCCCCCCC)C2]

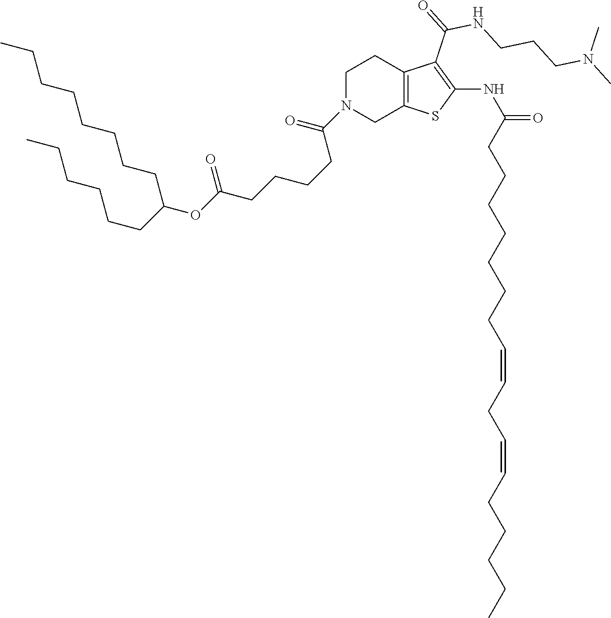 [SMILES: CCCCC/C=C\C/C=C\CCCCCCCC(=O)Nc1sc2c(c1C(=O)NCCCN(C)C)CCN(C(=O)CCCCC(=O)OC(CCCCCC)CCCCCCCC)C2]

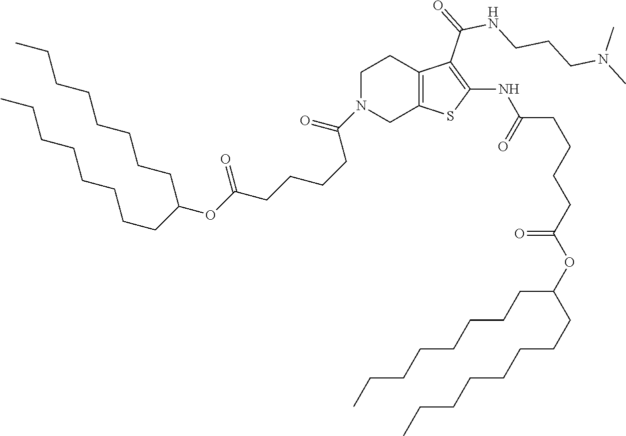 [SMILES: CCCCCCCCC(CCCCCCCC)OC(=O)CCCCC(=O)Nc1sc2c(c1C(=O)NCCCN(C)C)CCN(C(=O)CCCCC(=O)OC(CCCCCCCC)CCCCCCCC)C2]

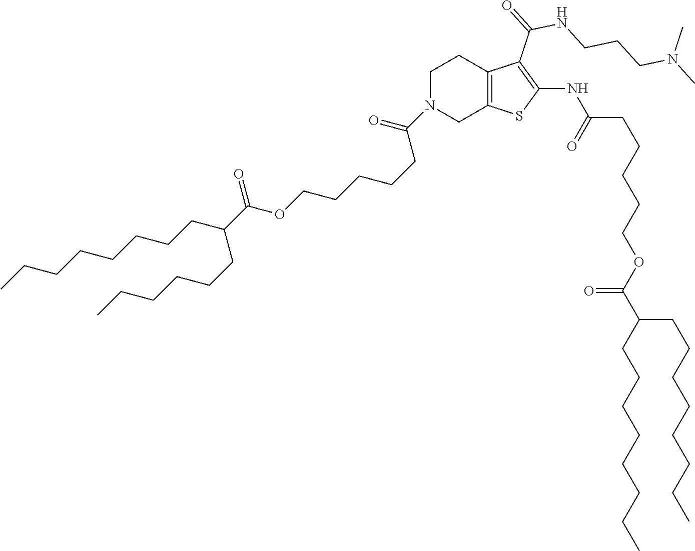 [SMILES: CCCCCCCCC(CCCCCC)C(=O)OCCCCCC(=O)N1CCc2c(sc(NC(=O)CCCCCOC(=O)C(CCCCCCCC)CCCCCCCC)c2C(=O)NCCCN(C)C)C1]

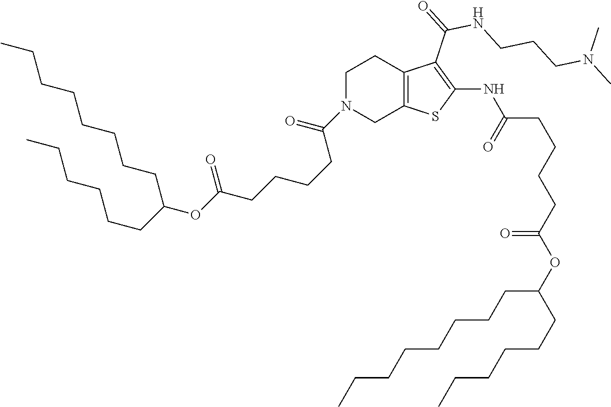 [SMILES: CCCCCCCCC(CCCCCC)OC(=O)CCCCC(=O)Nc1sc2c(c1C(=O)NCCCN(C)C)CCN(C(=O)CCCCC(=O)OC(CCCCCC)CCCCCCCC)C2]

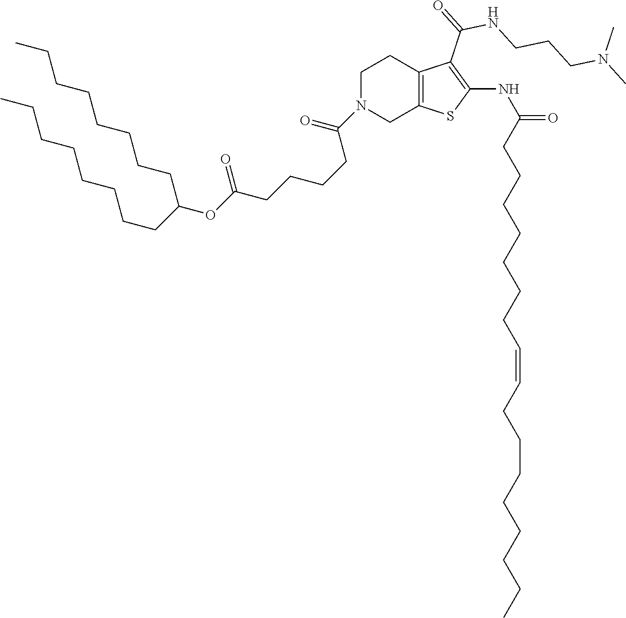 [SMILES: CCCCCCCC/C=C\CCCCCCCC(=O)Nc1sc2c(c1C(=O)NCCCN(C)C)CCN(C(=O)CCCCC(=O)OC(CCCCCCCC)CCCCCCCC)C2]

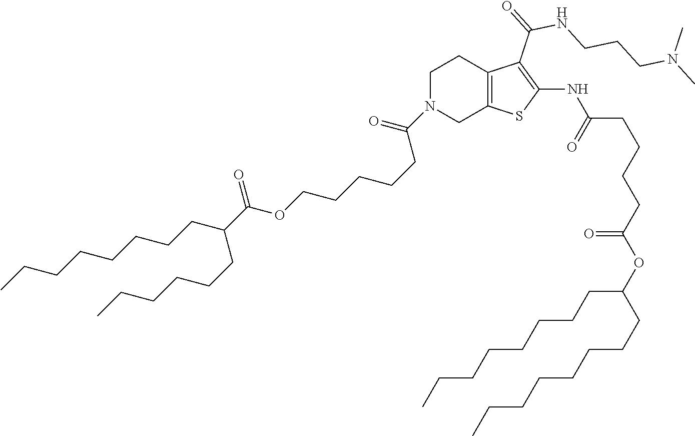 [SMILES: CCCCCCCCC(CCCCCCCC)OC(=O)CCCCC(=O)Nc1sc2c(c1C(=O)NCCCN(C)C)CCN(C(=O)CCCCCOC(=O)C(CCCCCC)CCCCCCCC)C2]

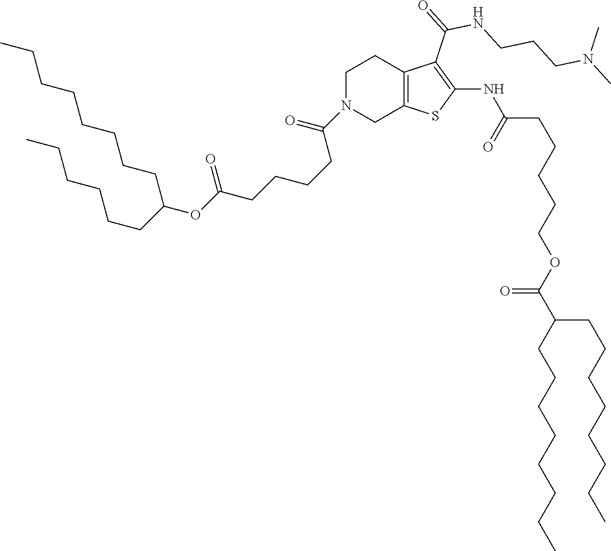 [SMILES: CCCCCCCCC(CCCCCC)OC(=O)CCCCC(=O)N1CCc2c(sc(NC(=O)CCCCCOC(=O)C(CCCCCCCC)CCCCCCCC)c2C(=O)NCCCN(C)C)C1]

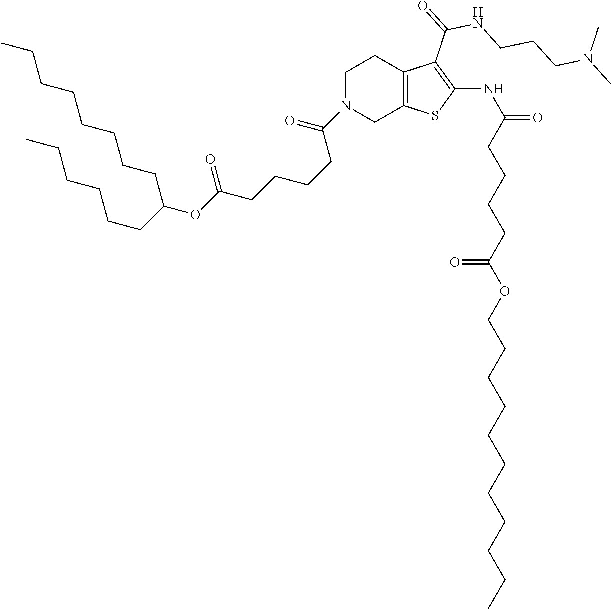 [SMILES: CCCCCCCCCCCOC(=O)CCCCC(=O)Nc1sc2c(c1C(=O)NCCCN(C)C)CCN(C(=O)CCCCC(=O)OC(CCCCCC)CCCCCCCC)C2]

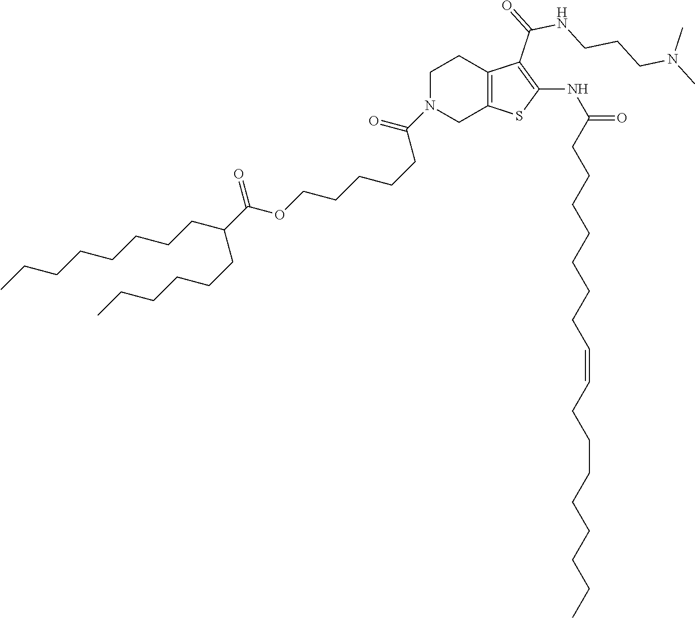 [SMILES: CCCCCCCC/C=C\CCCCCCCC(=O)Nc1sc2c(c1C(=O)NCCCN(C)C)CCN(C(=O)CCCCCOC(=O)C(CCCCCC)CCCCCCCC)C2]

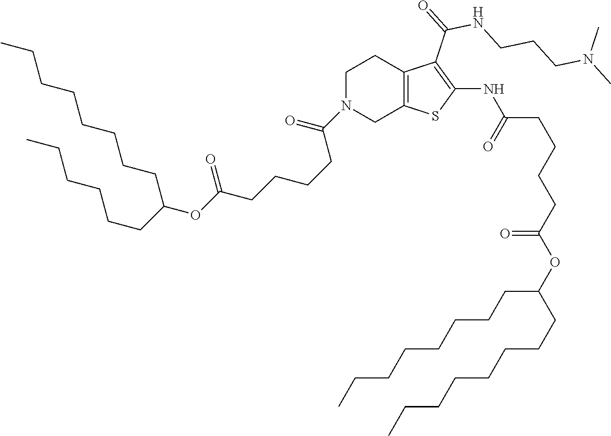 [SMILES: CCCCCCCCC(CCCCCCCC)OC(=O)CCCCC(=O)Nc1sc2c(c1C(=O)NCCCN(C)C)CCN(C(=O)CCCCC(=O)OC(CCCCCC)CCCCCCCC)C2]

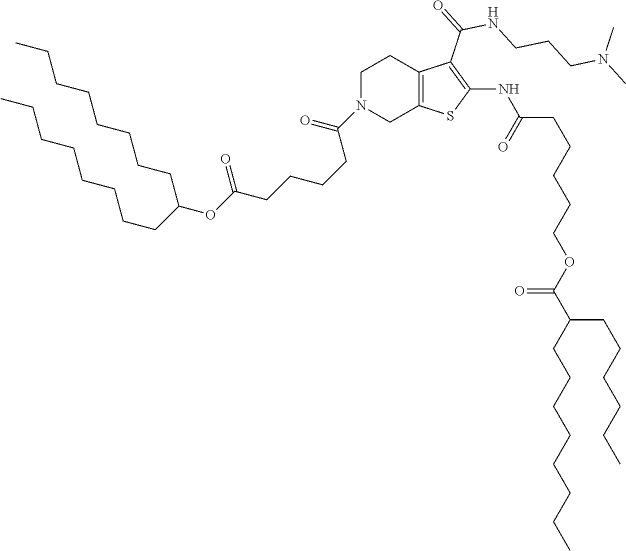 [SMILES: CCCCCCCCC(CCCCCCCC)OC(=O)CCCCC(=O)N1CCc2c(sc(NC(=O)CCCCCOC(=O)C(CCCCCC)CCCCCCCC)c2C(=O)NCCCN(C)C)C1]

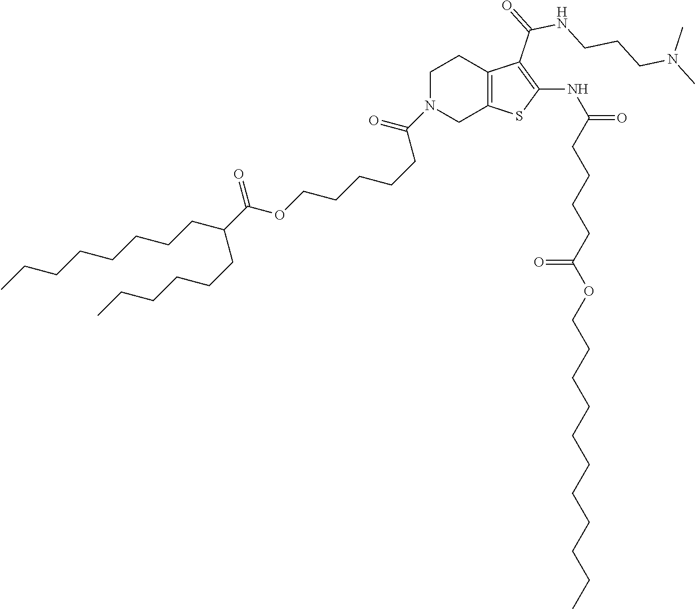 [SMILES: CCCCCCCCCCCOC(=O)CCCCC(=O)Nc1sc2c(c1C(=O)NCCCN(C)C)CCN(C(=O)CCCCCOC(=O)C(CCCCCC)CCCCCCCC)C2]

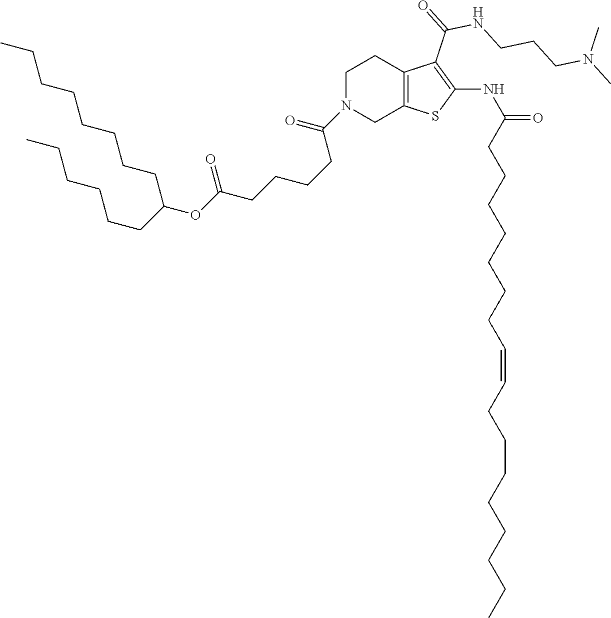 [SMILES: CCCCCCCC/C=C\CCCCCCCC(=O)Nc1sc2c(c1C(=O)NCCCN(C)C)CCN(C(=O)CCCCC(=O)OC(CCCCCC)CCCCCCCC)C2]

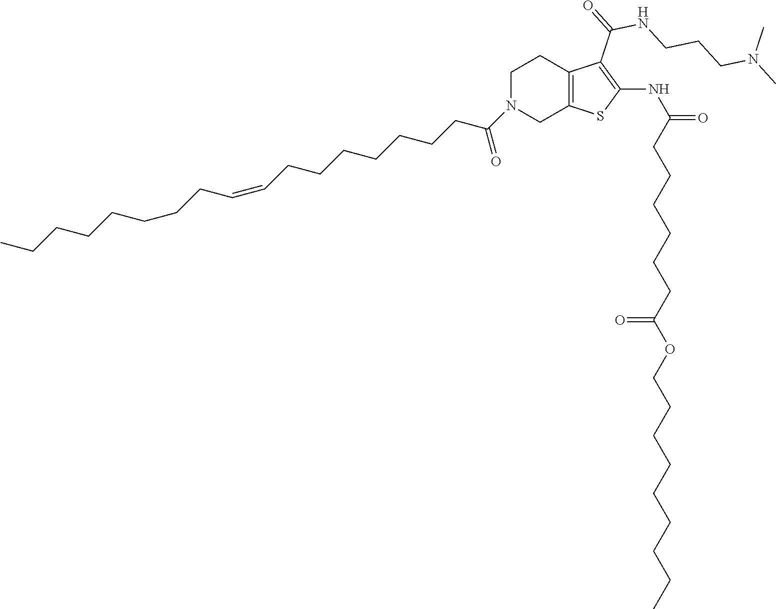 [SMILES: CCCCCCCC/C=C\CCCCCCCC(=O)N1CCc2c(sc(NC(=O)CCCCCCC(=O)OCCCCCCCCC)c2C(=O)NCCCN(C)C)C1]

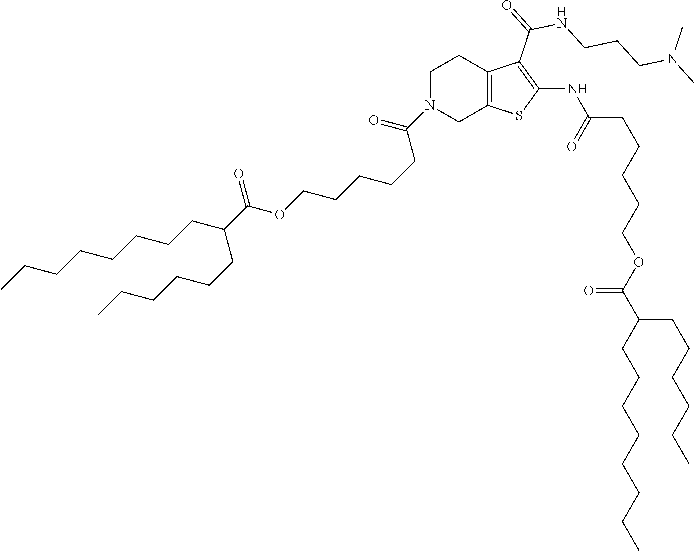 [SMILES: CCCCCCCCC(CCCCCC)C(=O)OCCCCCC(=O)Nc1sc2c(c1C(=O)NCCCN(C)C)CCN(C(=O)CCCCCOC(=O)C(CCCCCC)CCCCCCCC)C2]

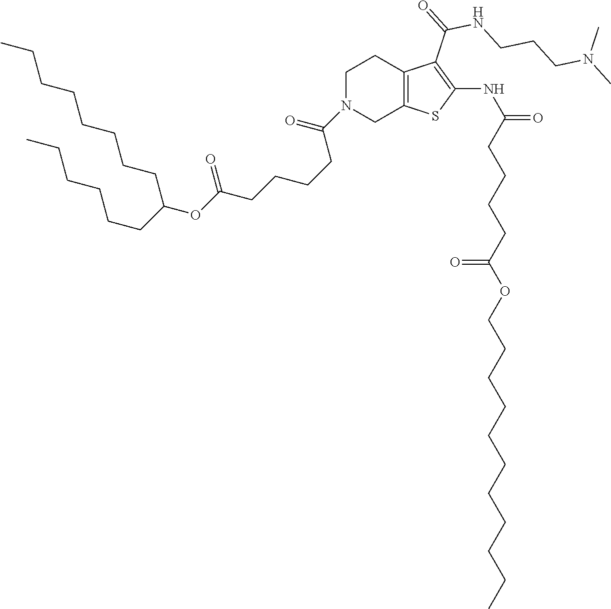 [SMILES: CCCCCCCCCCCOC(=O)CCCCC(=O)Nc1sc2c(c1C(=O)NCCCN(C)C)CCN(C(=O)CCCCC(=O)OC(CCCCCC)CCCCCCCC)C2]

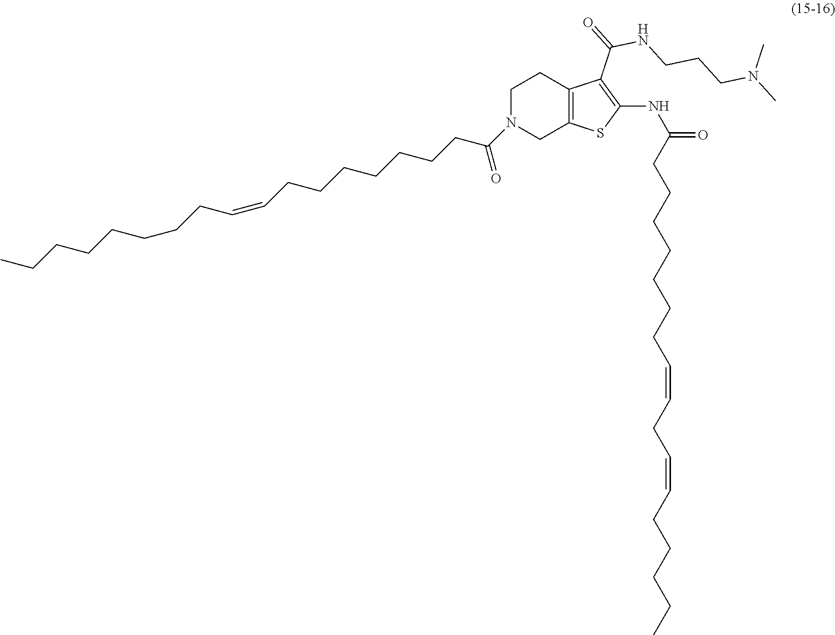 [SMILES: CCCCC/C=C\C/C=C\CCCCCCCC(=O)Nc1sc2c(c1C(=O)NCCCN(C)C)CCN(C(=O)CCCCCCC/C=C\CCCCCCCC)C2]

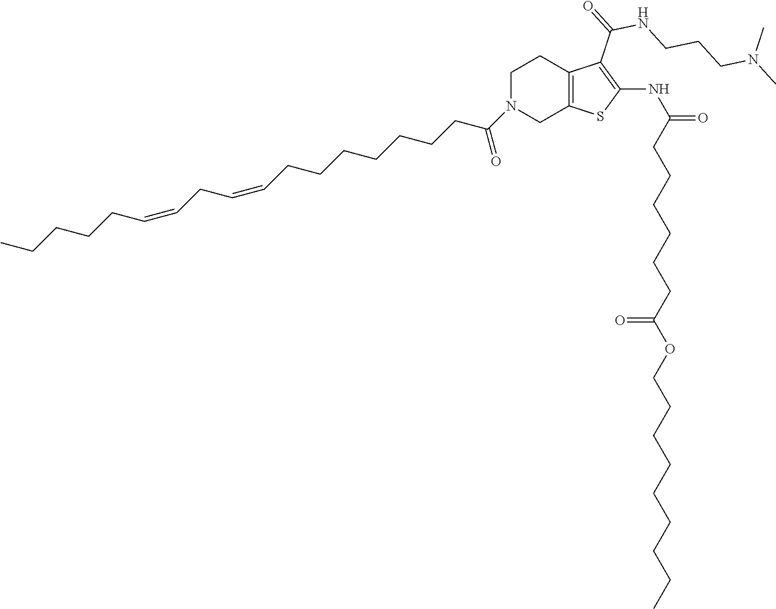 [SMILES: CCCCC/C=C\C/C=C\CCCCCCCC(=O)N1CCc2c(sc(NC(=O)CCCCCCC(=O)OCCCCCCCCC)c2C(=O)NCCCN(C)C)C1]

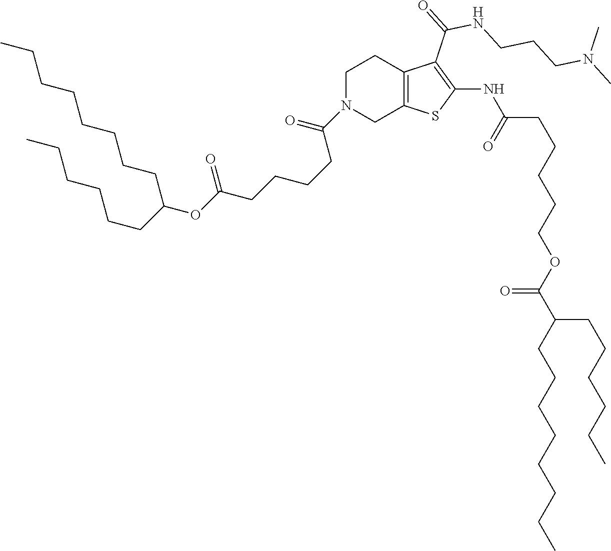 [SMILES: CCCCCCCCC(CCCCCC)OC(=O)CCCCC(=O)N1CCc2c(sc(NC(=O)CCCCCOC(=O)C(CCCCCC)CCCCCCCC)c2C(=O)NCCCN(C)C)C1]

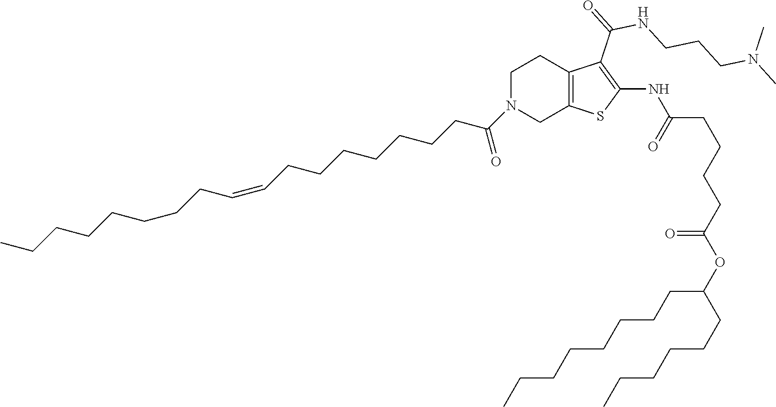 [SMILES: CCCCCCCC/C=C\CCCCCCCC(=O)N1CCc2c(sc(NC(=O)CCCCC(=O)OC(CCCCCC)CCCCCCCC)c2C(=O)NCCCN(C)C)C1]

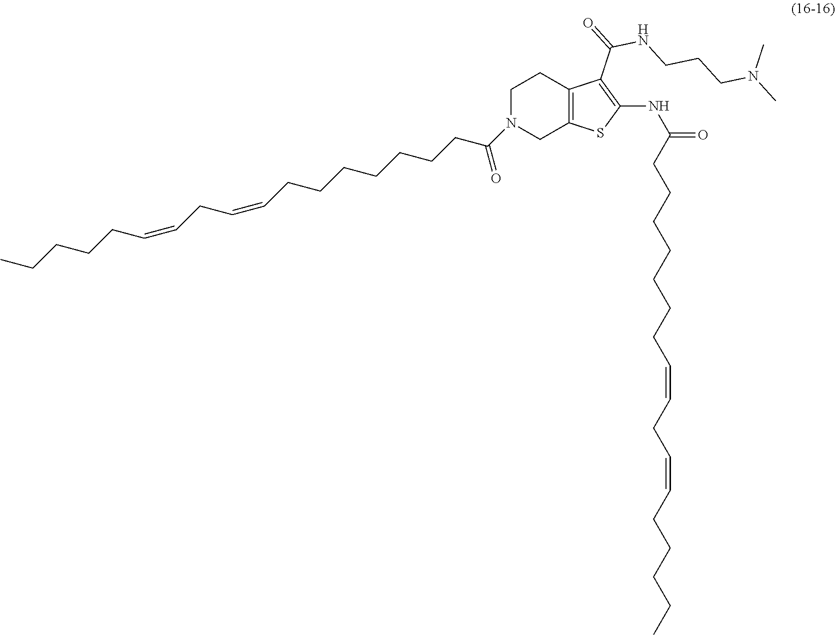 [SMILES: CCCCC/C=C\C/C=C\CCCCCCCC(=O)Nc1sc2c(c1C(=O)NCCCN(C)C)CCN(C(=O)CCCCCCC/C=C\C/C=C\CCCCC)C2]

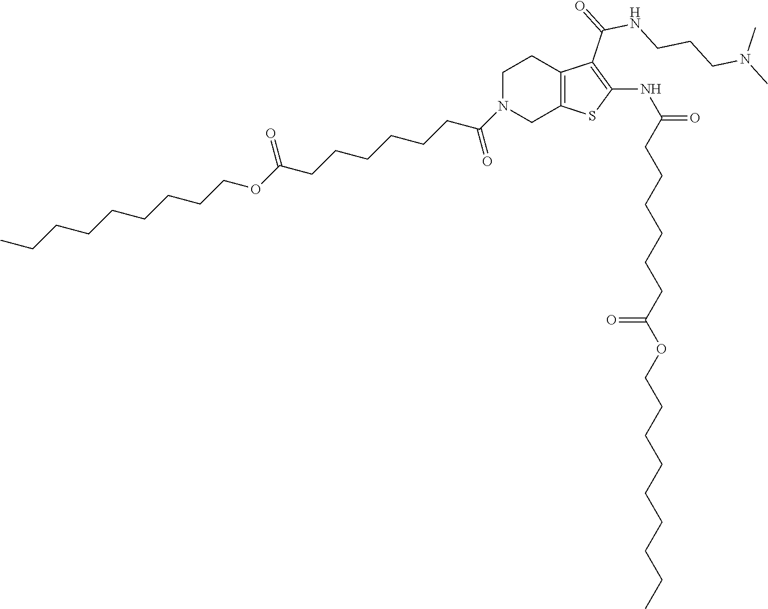 [SMILES: CCCCCCCCCOC(=O)CCCCCCC(=O)Nc1sc2c(c1C(=O)NCCCN(C)C)CCN(C(=O)CCCCCCC(=O)OCCCCCCCCC)C2]

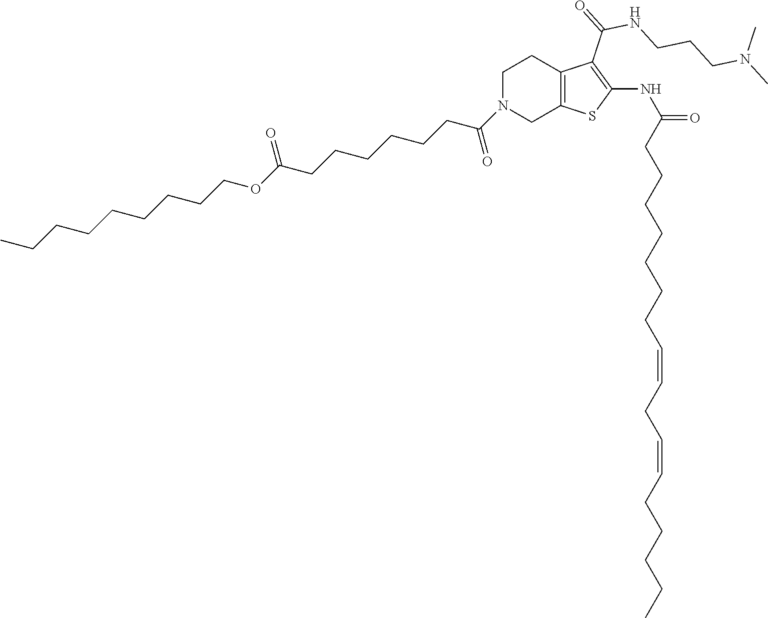 [SMILES: CCCCC/C=C\C/C=C\CCCCCCCC(=O)Nc1sc2c(c1C(=O)NCCCN(C)C)CCN(C(=O)CCCCCCC(=O)OCCCCCCCCC)C2]

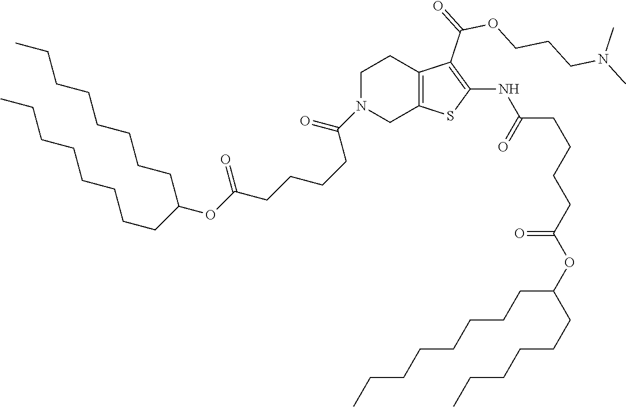 [SMILES: CCCCCCCCC(CCCCCC)OC(=O)CCCCC(=O)Nc1sc2c(c1C(=O)OCCCN(C)C)CCN(C(=O)CCCCC(=O)OC(CCCCCCCC)CCCCCCCC)C2]

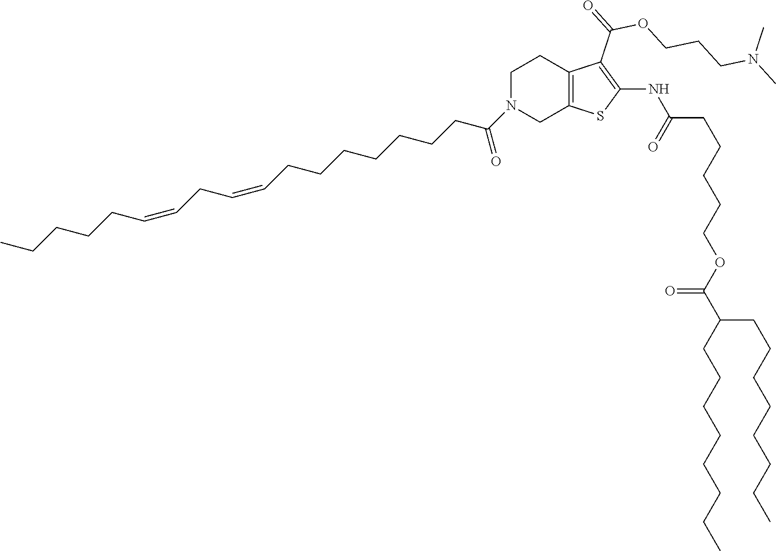 [SMILES: CCCCC/C=C\C/C=C\CCCCCCCC(=O)N1CCc2c(sc(NC(=O)CCCCCOC(=O)C(CCCCCCCC)CCCCCCCC)c2C(=O)OCCCN(C)C)C1]

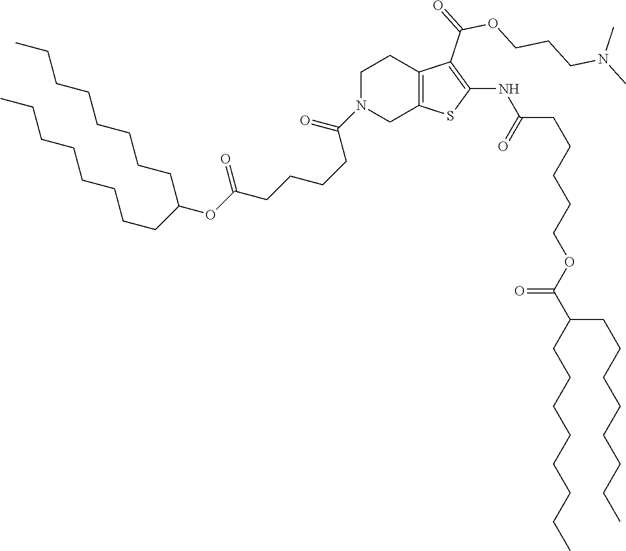 [SMILES: CCCCCCCCC(CCCCCCCC)OC(=O)CCCCC(=O)N1CCc2c(sc(NC(=O)CCCCCOC(=O)C(CCCCCCCC)CCCCCCCC)c2C(=O)OCCCN(C)C)C1]

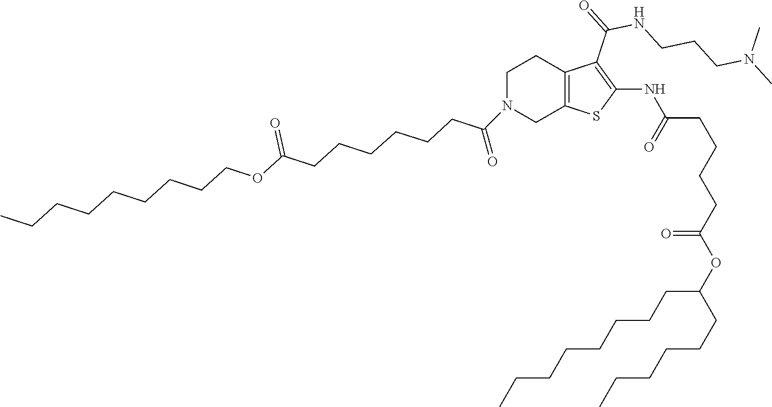 [SMILES: CCCCCCCCCOC(=O)CCCCCCC(=O)N1CCc2c(sc(NC(=O)CCCCC(=O)OC(CCCCCC)CCCCCCCC)c2C(=O)NCCCN(C)C)C1]

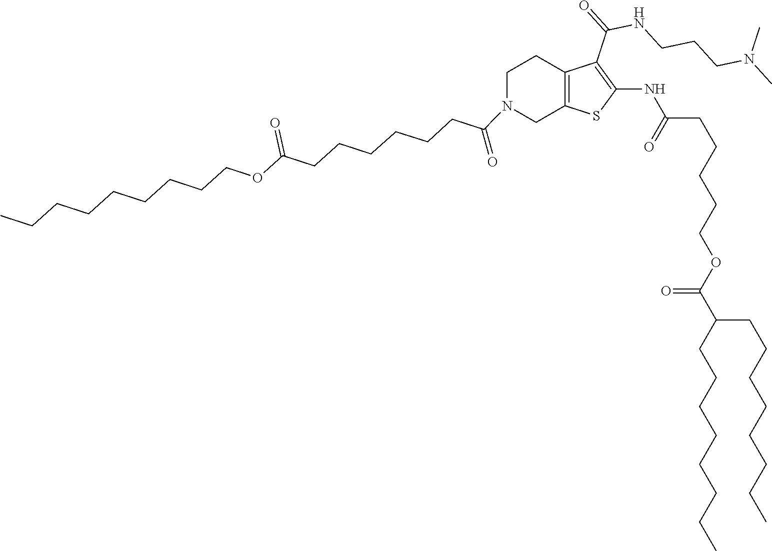 [SMILES: CCCCCCCCCOC(=O)CCCCCCC(=O)N1CCc2c(sc(NC(=O)CCCCCOC(=O)C(CCCCCCCC)CCCCCCCC)c2C(=O)NCCCN(C)C)C1]

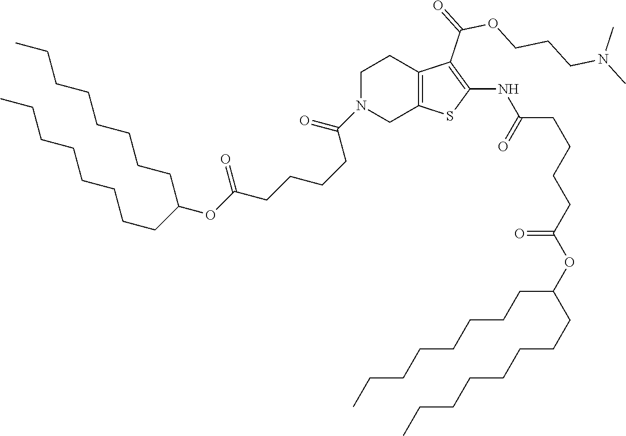 [SMILES: CCCCCCCCC(CCCCCCCC)OC(=O)CCCCC(=O)Nc1sc2c(c1C(=O)OCCCN(C)C)CCN(C(=O)CCCCC(=O)OC(CCCCCCCC)CCCCCCCC)C2]

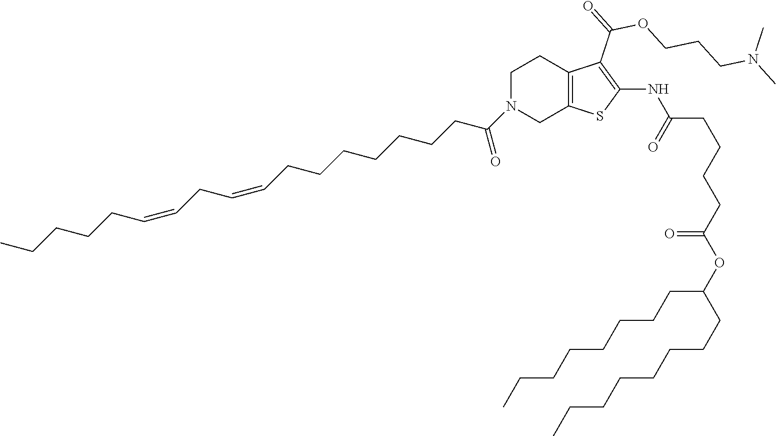 [SMILES: CCCCC/C=C\C/C=C\CCCCCCCC(=O)N1CCc2c(sc(NC(=O)CCCCC(=O)OC(CCCCCCCC)CCCCCCCC)c2C(=O)OCCCN(C)C)C1]

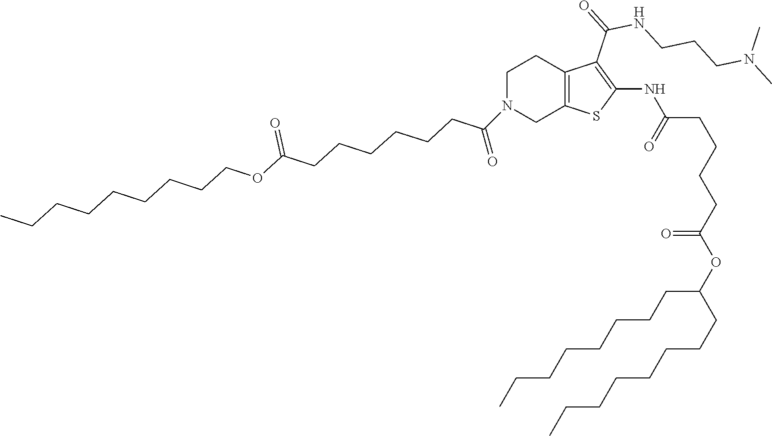 [SMILES: CCCCCCCCCOC(=O)CCCCCCC(=O)N1CCc2c(sc(NC(=O)CCCCC(=O)OC(CCCCCCCC)CCCCCCCC)c2C(=O)NCCCN(C)C)C1]

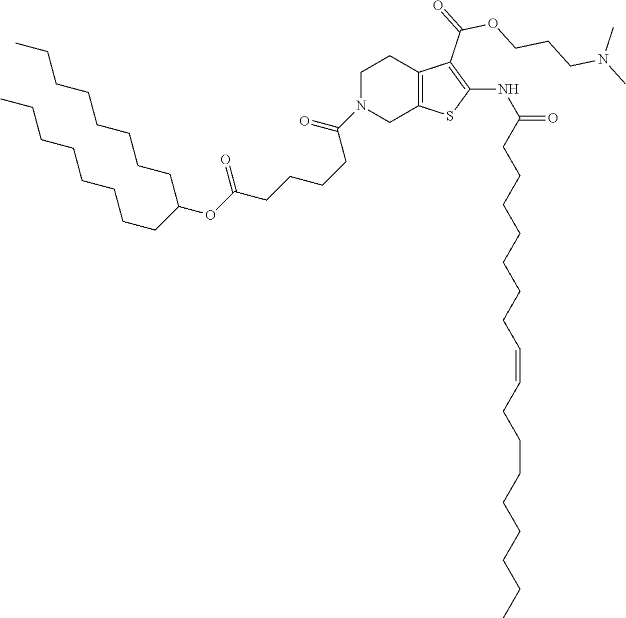 [SMILES: CCCCCCCC/C=C\CCCCCCCC(=O)Nc1sc2c(c1C(=O)OCCCN(C)C)CCN(C(=O)CCCCC(=O)OC(CCCCCCCC)CCCCCCCC)C2]

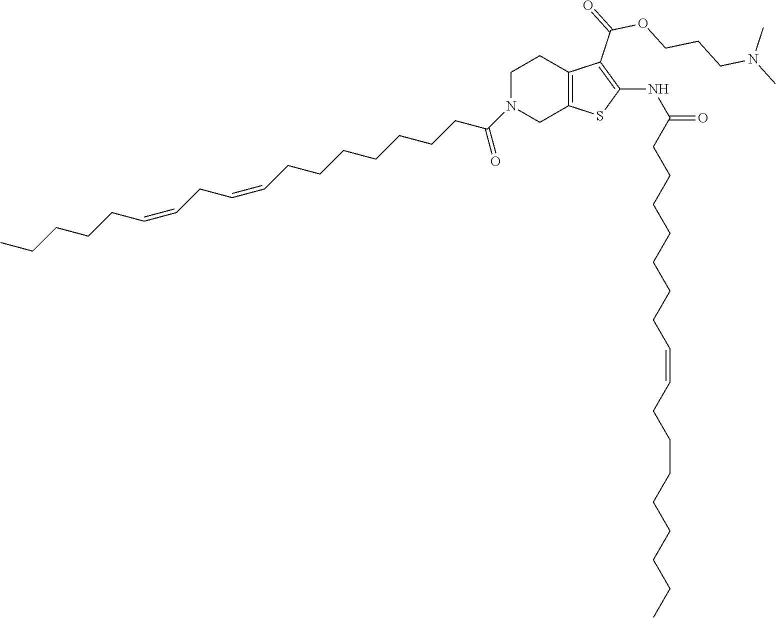 [SMILES: CCCCC/C=C\C/C=C\CCCCCCCC(=O)N1CCc2c(sc(NC(=O)CCCCCCC/C=C\CCCCCCCC)c2C(=O)OCCCN(C)C)C1]

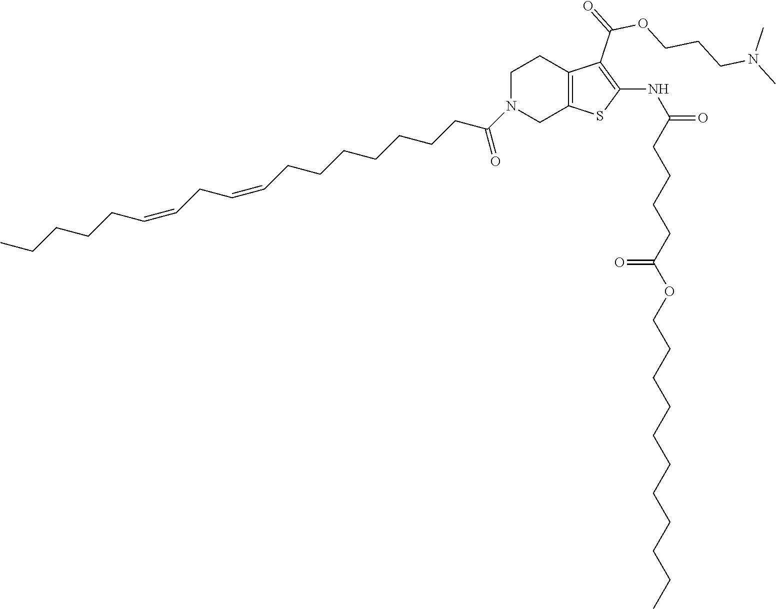 [SMILES: CCCCC/C=C\C/C=C\CCCCCCCC(=O)N1CCc2c(sc(NC(=O)CCCCC(=O)OCCCCCCCCCCC)c2C(=O)OCCCN(C)C)C1]

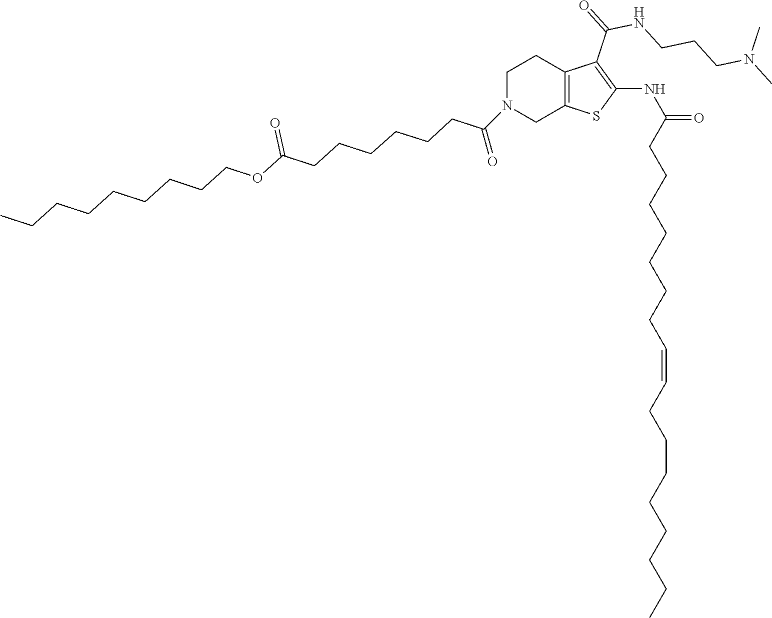 [SMILES: CCCCCCCC/C=C\CCCCCCCC(=O)Nc1sc2c(c1C(=O)NCCCN(C)C)CCN(C(=O)CCCCCCC(=O)OCCCCCCCCC)C2]

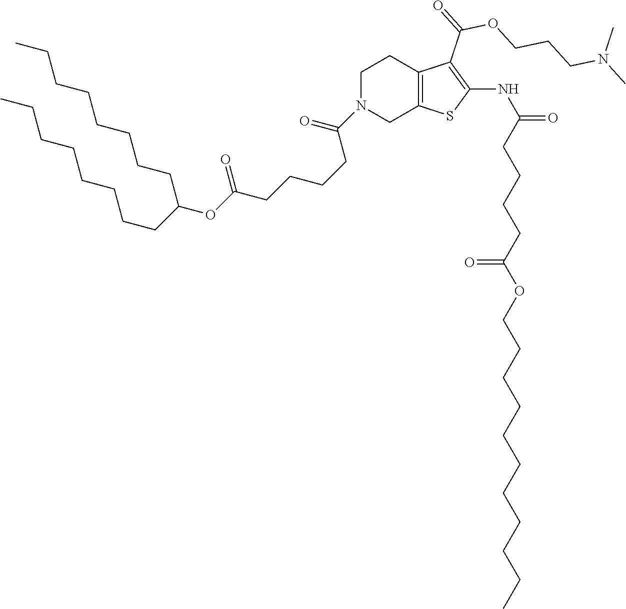 [SMILES: CCCCCCCCCCCOC(=O)CCCCC(=O)Nc1sc2c(c1C(=O)OCCCN(C)C)CCN(C(=O)CCCCC(=O)OC(CCCCCCCC)CCCCCCCC)C2]

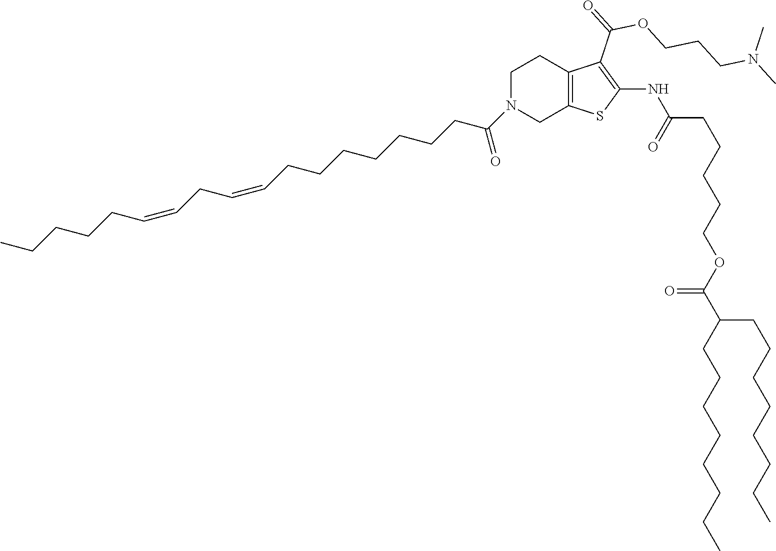 [SMILES: CCCCC/C=C\C/C=C\CCCCCCCC(=O)N1CCc2c(sc(NC(=O)CCCCCOC(=O)C(CCCCCCCC)CCCCCCCC)c2C(=O)OCCCN(C)C)C1]

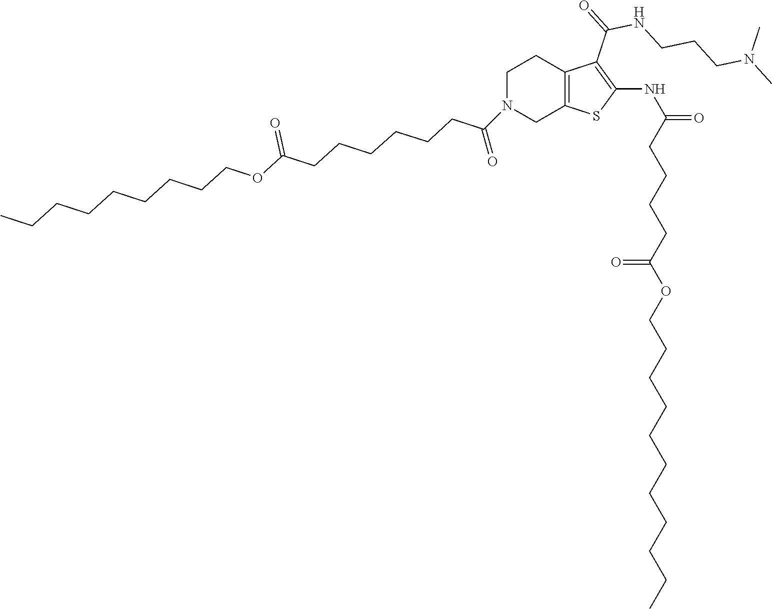 [SMILES: CCCCCCCCCCCOC(=O)CCCCC(=O)Nc1sc2c(c1C(=O)NCCCN(C)C)CCN(C(=O)CCCCCCC(=O)OCCCCCCCCC)C2]

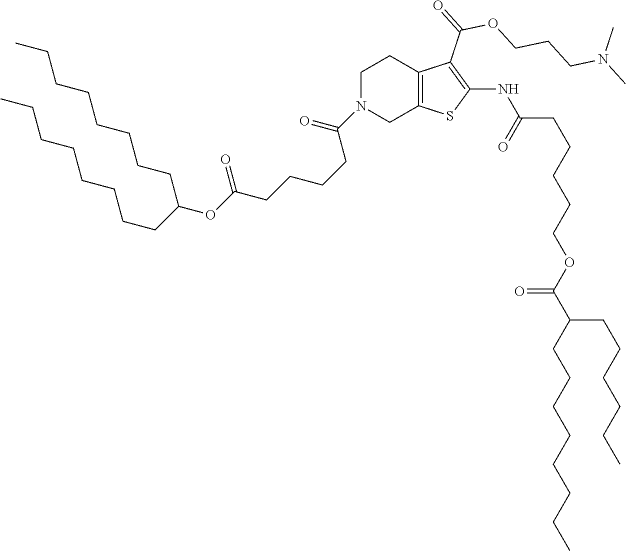 [SMILES: CCCCCCCCC(CCCCCCCC)OC(=O)CCCCC(=O)N1CCc2c(sc(NC(=O)CCCCCOC(=O)C(CCCCCC)CCCCCCCC)c2C(=O)OCCCN(C)C)C1]

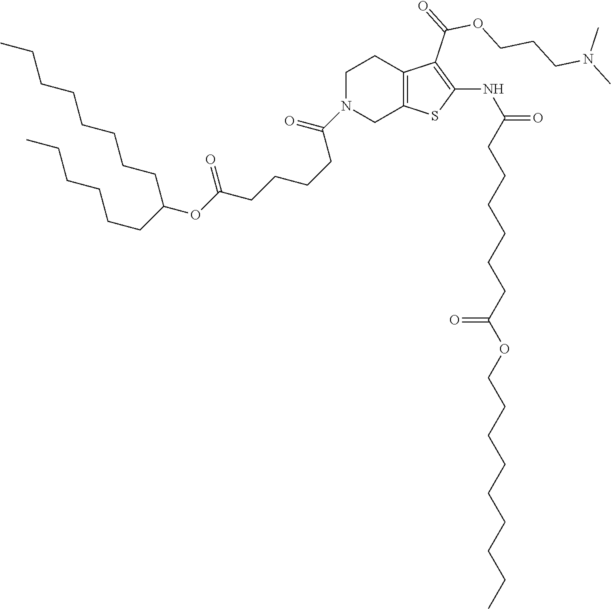 [SMILES: CCCCCCCCCOC(=O)CCCCCCC(=O)Nc1sc2c(c1C(=O)OCCCN(C)C)CCN(C(=O)CCCCC(=O)OC(CCCCCC)CCCCCCCC)C2]

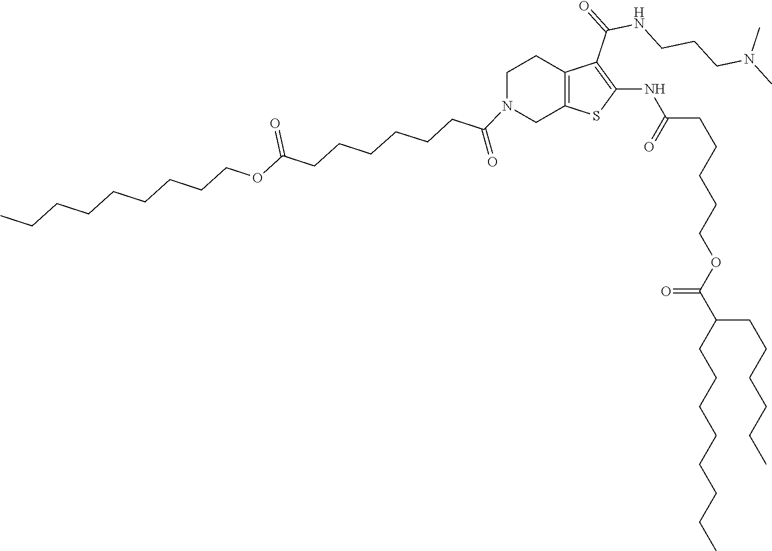 [SMILES: CCCCCCCCCOC(=O)CCCCCCC(=O)N1CCc2c(sc(NC(=O)CCCCCOC(=O)C(CCCCCC)CCCCCCCC)c2C(=O)NCCCN(C)C)C1]

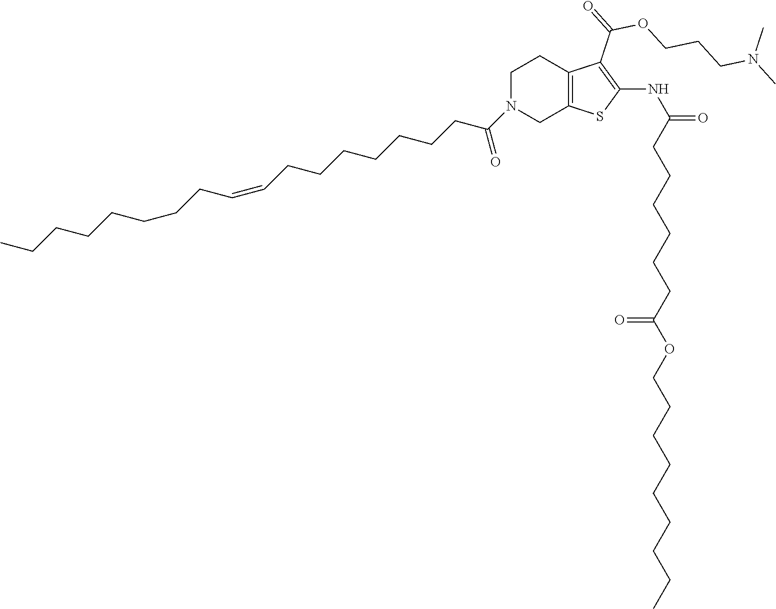 [SMILES: CCCCCCCC/C=C\CCCCCCCC(=O)N1CCc2c(sc(NC(=O)CCCCCCC(=O)OCCCCCCCCC)c2C(=O)OCCCN(C)C)C1]

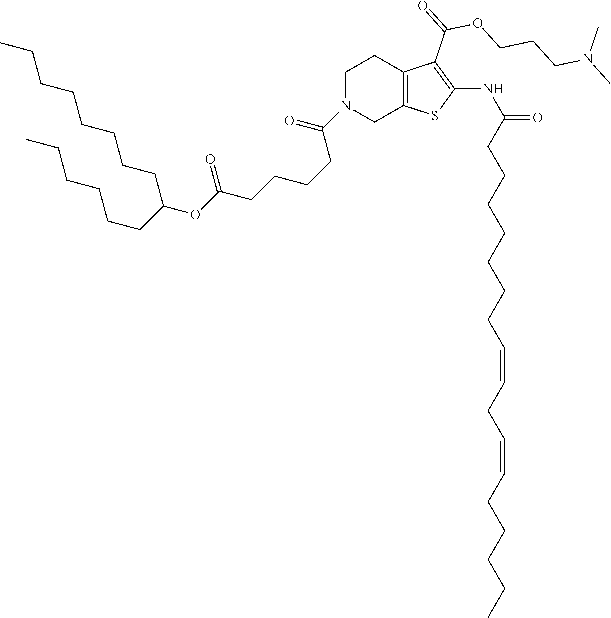 [SMILES: CCCCC/C=C\C/C=C\CCCCCCCC(=O)Nc1sc2c(c1C(=O)OCCCN(C)C)CCN(C(=O)CCCCC(=O)OC(CCCCCC)CCCCCCCC)C2]

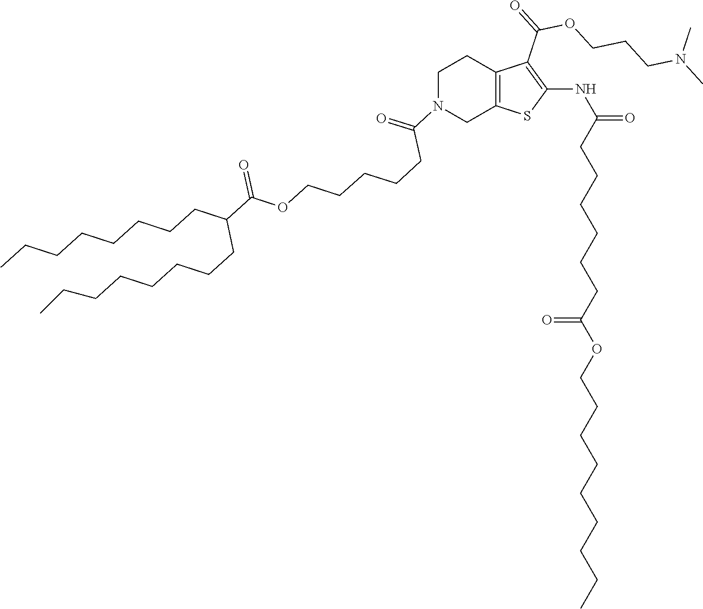 [SMILES: CCCCCCCCCOC(=O)CCCCCCC(=O)Nc1sc2c(c1C(=O)OCCCN(C)C)CCN(C(=O)CCCCCOC(=O)C(CCCCCCCC)CCCCCCCC)C2]

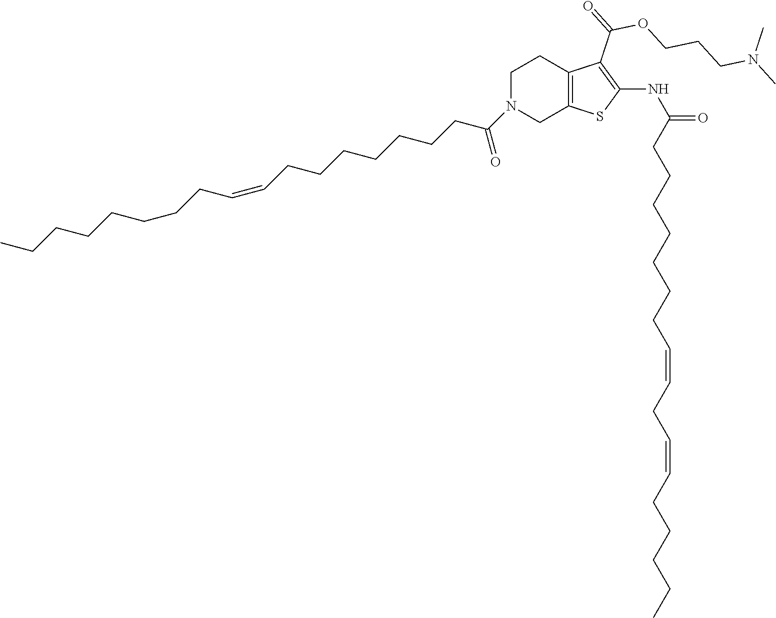 [SMILES: CCCCC/C=C\C/C=C\CCCCCCCC(=O)Nc1sc2c(c1C(=O)OCCCN(C)C)CCN(C(=O)CCCCCCC/C=C\CCCCCCCC)C2]

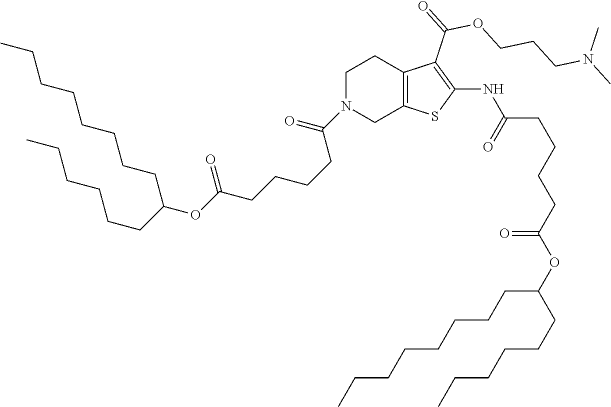 [SMILES: CCCCCCCCC(CCCCCC)OC(=O)CCCCC(=O)Nc1sc2c(c1C(=O)OCCCN(C)C)CCN(C(=O)CCCCC(=O)OC(CCCCCC)CCCCCCCC)C2]

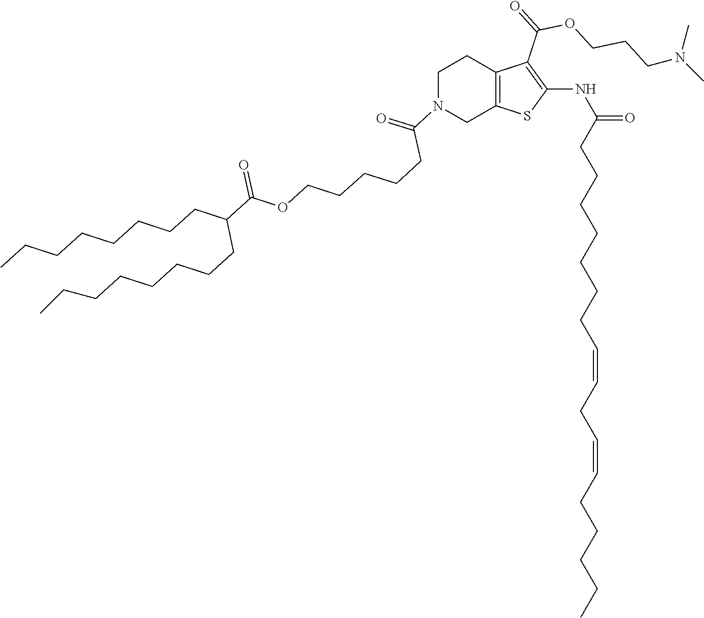 [SMILES: CCCCC/C=C\C/C=C\CCCCCCCC(=O)Nc1sc2c(c1C(=O)OCCCN(C)C)CCN(C(=O)CCCCCOC(=O)C(CCCCCCCC)CCCCCCCC)C2]

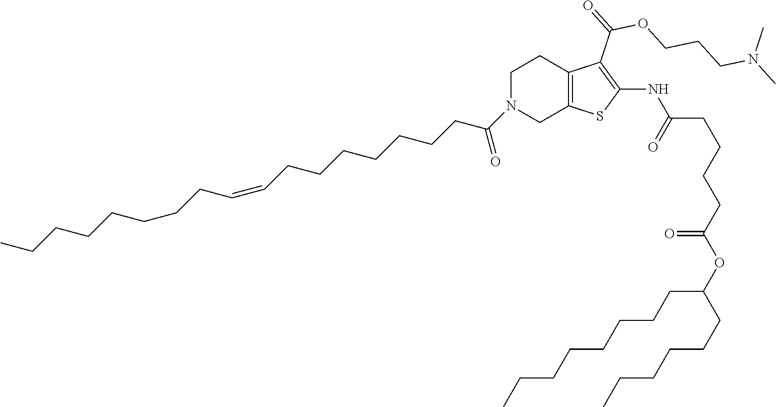 [SMILES: CCCCCCCC/C=C\CCCCCCCC(=O)N1CCc2c(sc(NC(=O)CCCCC(=O)OC(CCCCCC)CCCCCCCC)c2C(=O)OCCCN(C)C)C1]

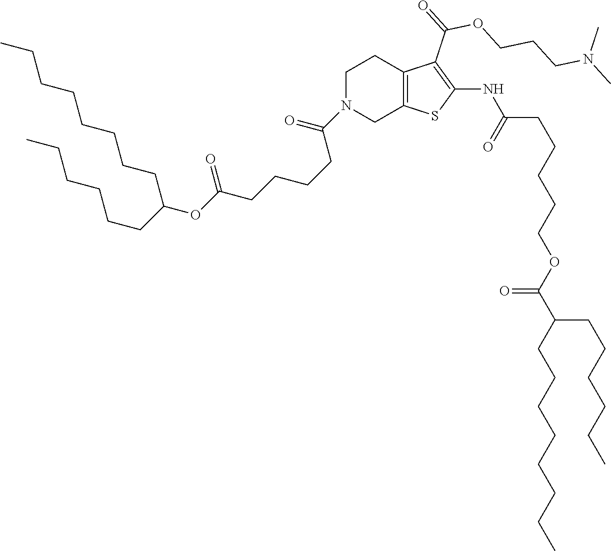 [SMILES: CCCCCCCCC(CCCCCC)OC(=O)CCCCC(=O)N1CCc2c(sc(NC(=O)CCCCCOC(=O)C(CCCCCC)CCCCCCCC)c2C(=O)OCCCN(C)C)C1]

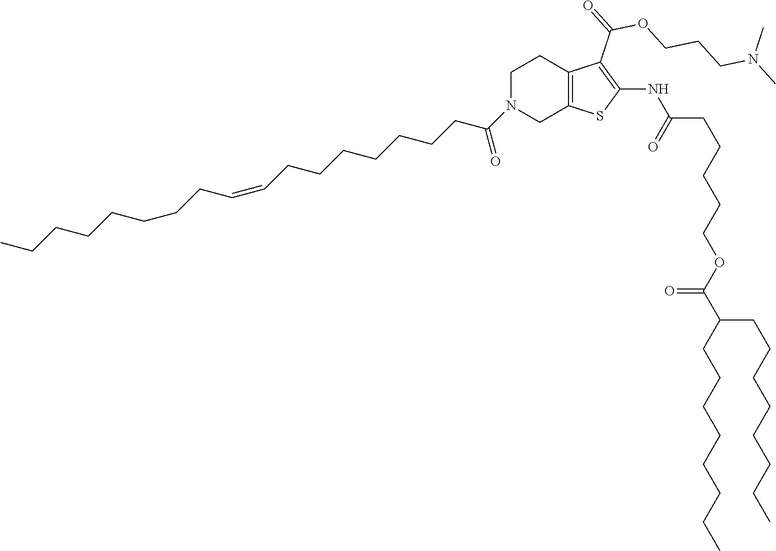 [SMILES: CCCCCCCC/C=C\CCCCCCCC(=O)N1CCc2c(sc(NC(=O)CCCCCOC(=O)C(CCCCCCCC)CCCCCCCC)c2C(=O)OCCCN(C)C)C1]

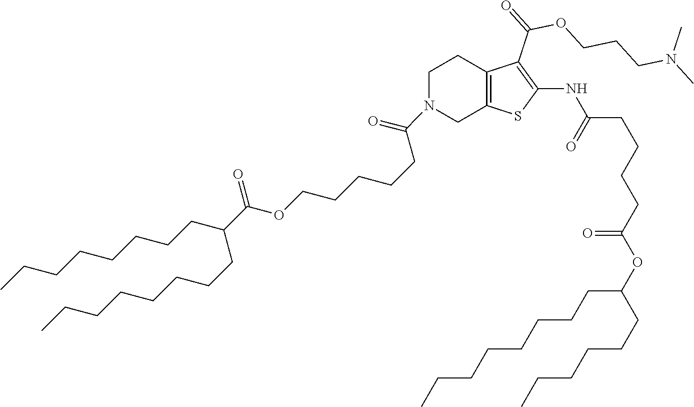 [SMILES: CCCCCCCCC(CCCCCC)OC(=O)CCCCC(=O)Nc1sc2c(c1C(=O)OCCCN(C)C)CCN(C(=O)CCCCCOC(=O)C(CCCCCCCC)CCCCCCCC)C2]

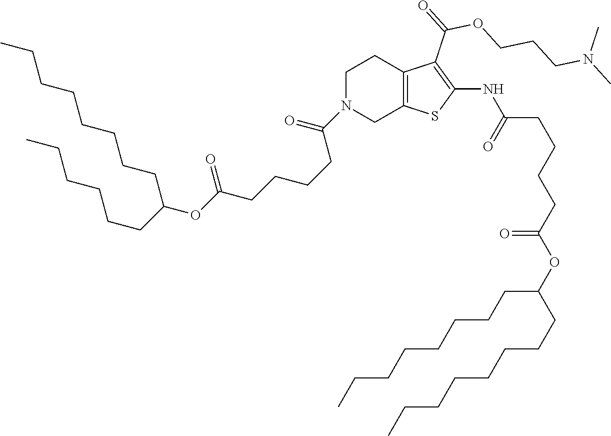 [SMILES: CCCCCCCCC(CCCCCCCC)OC(=O)CCCCC(=O)Nc1sc2c(c1C(=O)OCCCN(C)C)CCN(C(=O)CCCCC(=O)OC(CCCCCC)CCCCCCCC)C2]

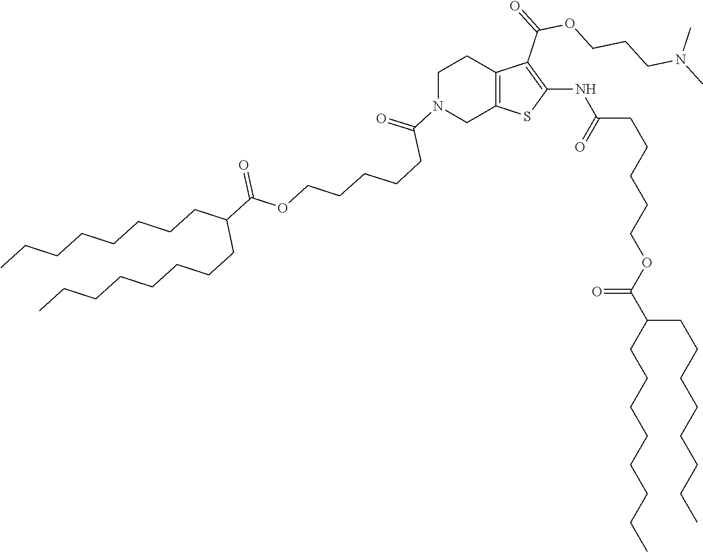 [SMILES: CCCCCCCCC(CCCCCCCC)C(=O)OCCCCCC(=O)Nc1sc2c(c1C(=O)OCCCN(C)C)CCN(C(=O)CCCCCOC(=O)C(CCCCCCCC)CCCCCCCC)C2]

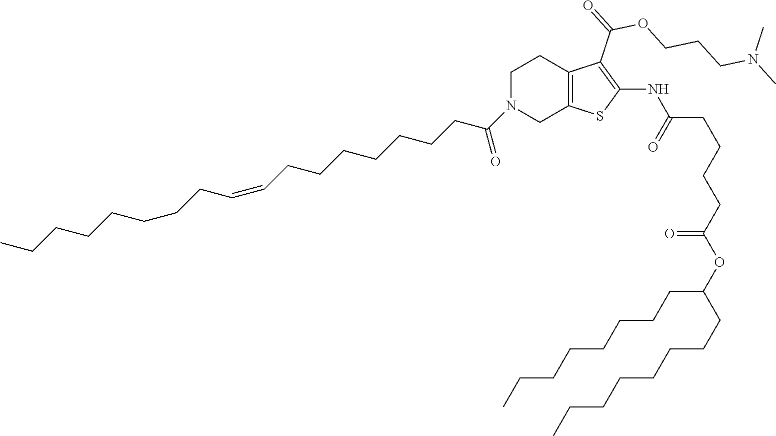 [SMILES: CCCCCCCC/C=C\CCCCCCCC(=O)N1CCc2c(sc(NC(=O)CCCCC(=O)OC(CCCCCCCC)CCCCCCCC)c2C(=O)OCCCN(C)C)C1]

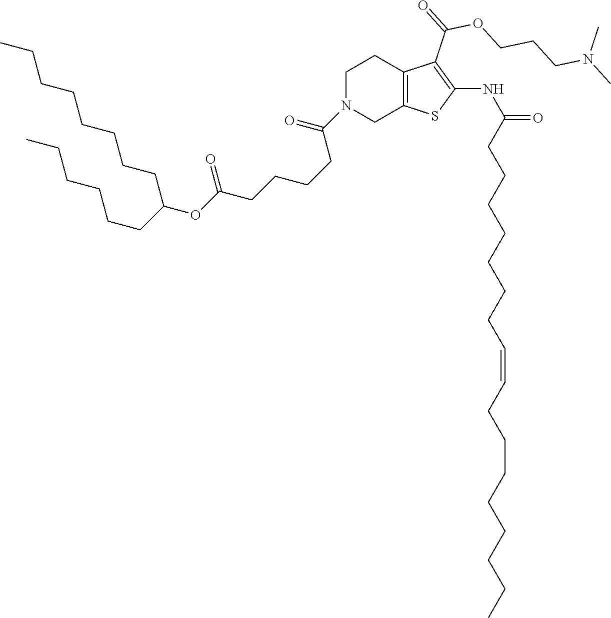 [SMILES: CCCCCCCC/C=C\CCCCCCCC(=O)Nc1sc2c(c1C(=O)OCCCN(C)C)CCN(C(=O)CCCCC(=O)OC(CCCCCC)CCCCCCCC)C2]

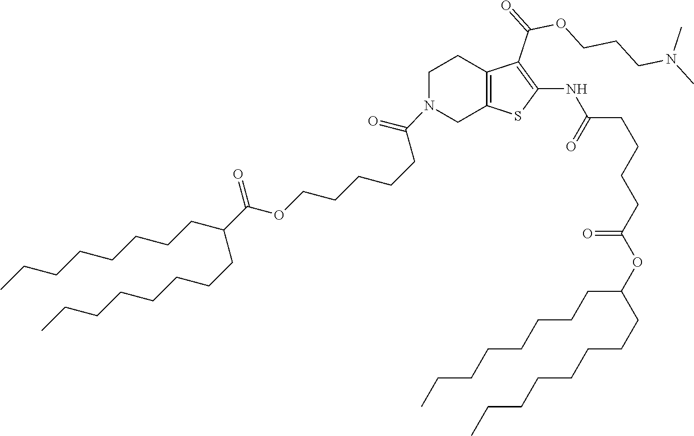 [SMILES: CCCCCCCCC(CCCCCCCC)OC(=O)CCCCC(=O)Nc1sc2c(c1C(=O)OCCCN(C)C)CCN(C(=O)CCCCCOC(=O)C(CCCCCCCC)CCCCCCCC)C2]

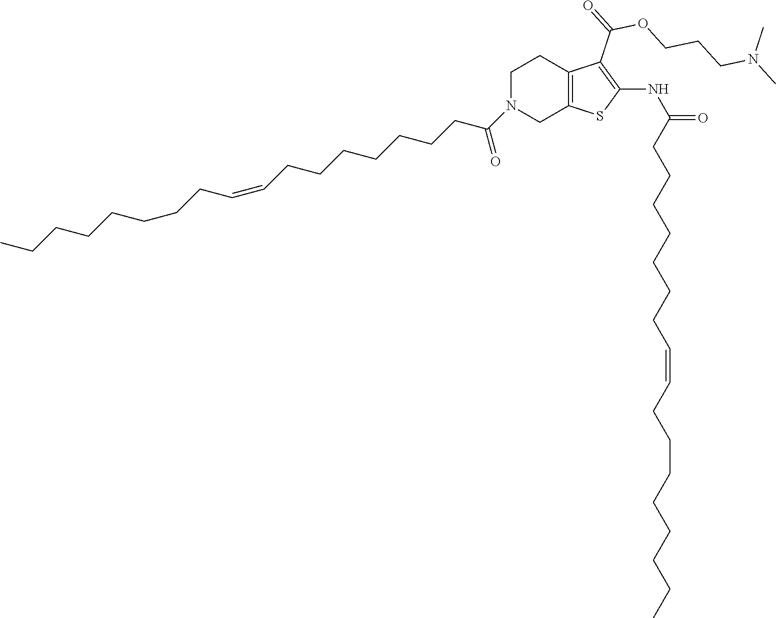 [SMILES: CCCCCCCC/C=C\CCCCCCCC(=O)Nc1sc2c(c1C(=O)OCCCN(C)C)CCN(C(=O)CCCCCCC/C=C\CCCCCCCC)C2]

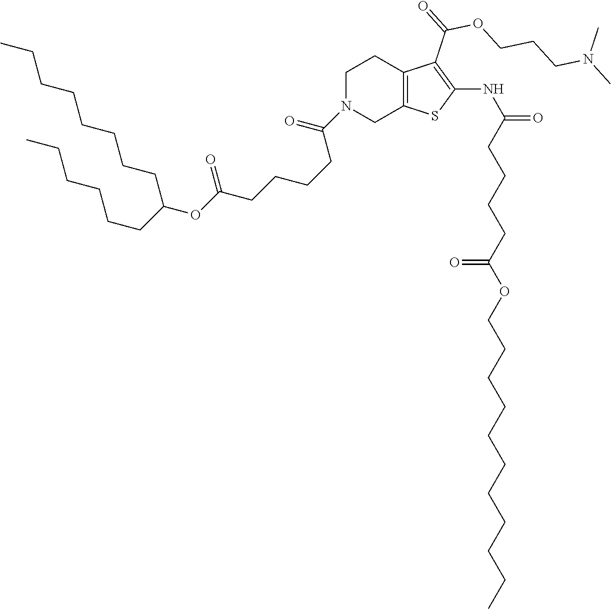 [SMILES: CCCCCCCCCCCOC(=O)CCCCC(=O)Nc1sc2c(c1C(=O)OCCCN(C)C)CCN(C(=O)CCCCC(=O)OC(CCCCCC)CCCCCCCC)C2]

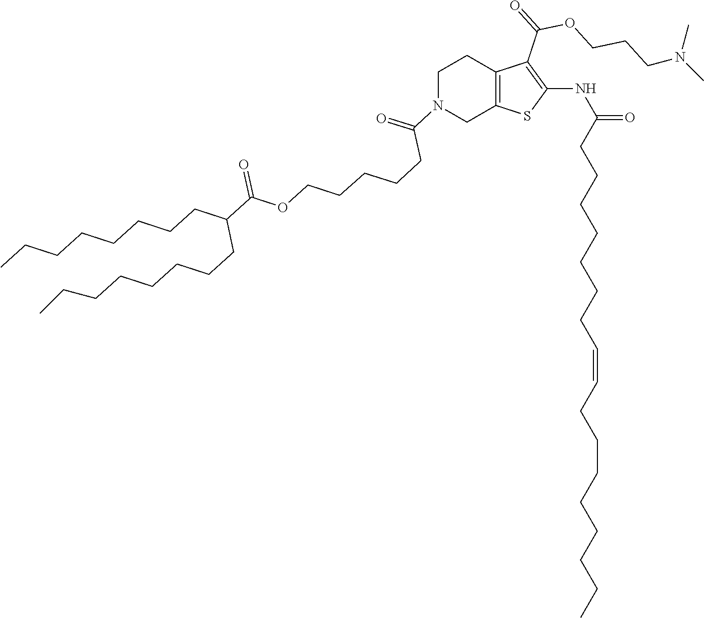 [SMILES: CCCCCCCC/C=C\CCCCCCCC(=O)Nc1sc2c(c1C(=O)OCCCN(C)C)CCN(C(=O)CCCCCOC(=O)C(CCCCCCCC)CCCCCCCC)C2]

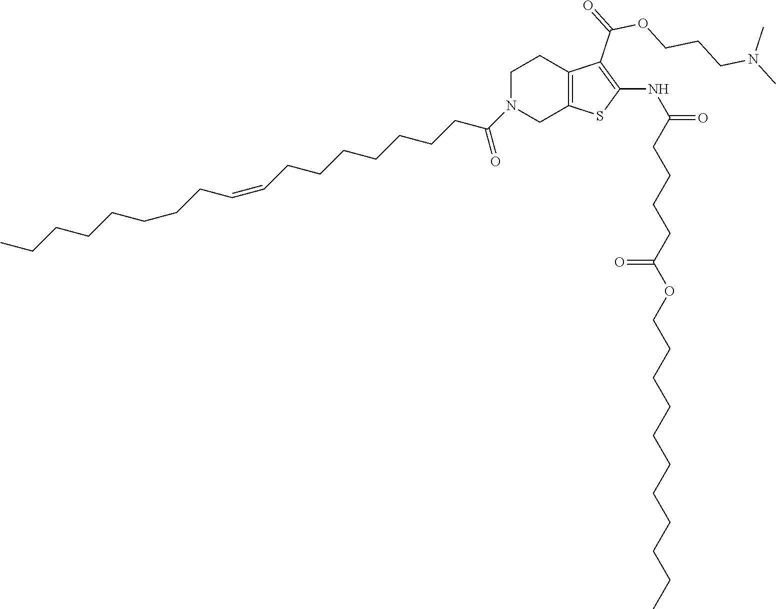 [SMILES: CCCCCCCC/C=C\CCCCCCCC(=O)N1CCc2c(sc(NC(=O)CCCCC(=O)OCCCCCCCCCCC)c2C(=O)OCCCN(C)C)C1]

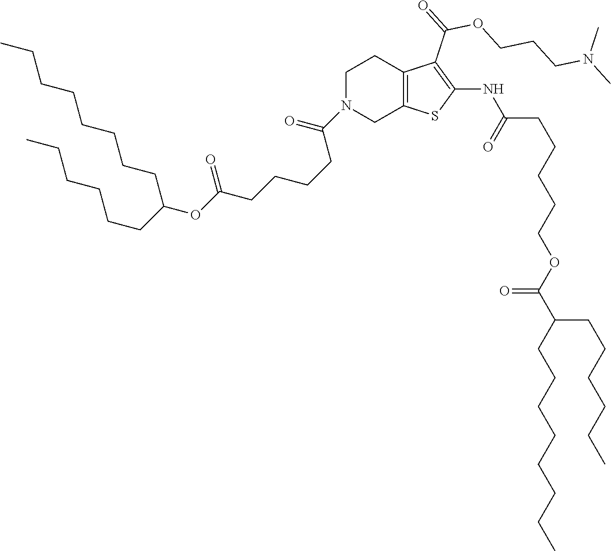 [SMILES: CCCCCCCCC(CCCCCC)OC(=O)CCCCC(=O)N1CCc2c(sc(NC(=O)CCCCCOC(=O)C(CCCCCC)CCCCCCCC)c2C(=O)OCCCN(C)C)C1]

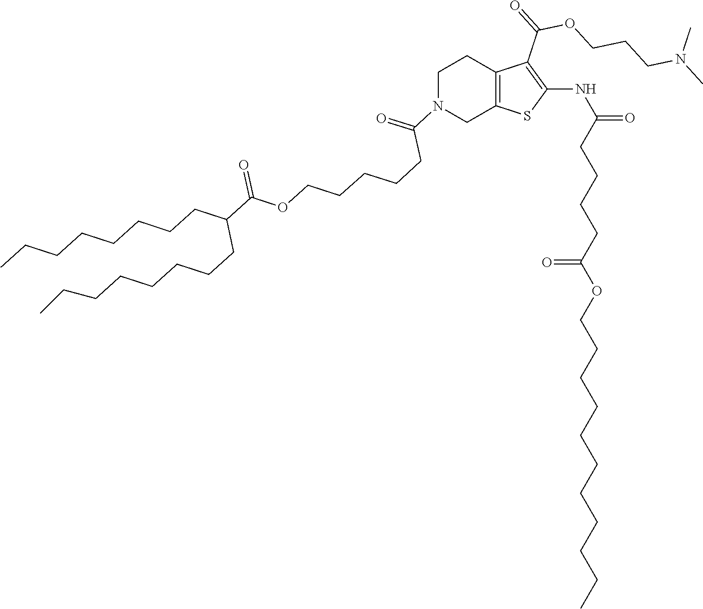 [SMILES: CCCCCCCCCCCOC(=O)CCCCC(=O)Nc1sc2c(c1C(=O)OCCCN(C)C)CCN(C(=O)CCCCCOC(=O)C(CCCCCCCC)CCCCCCCC)C2]

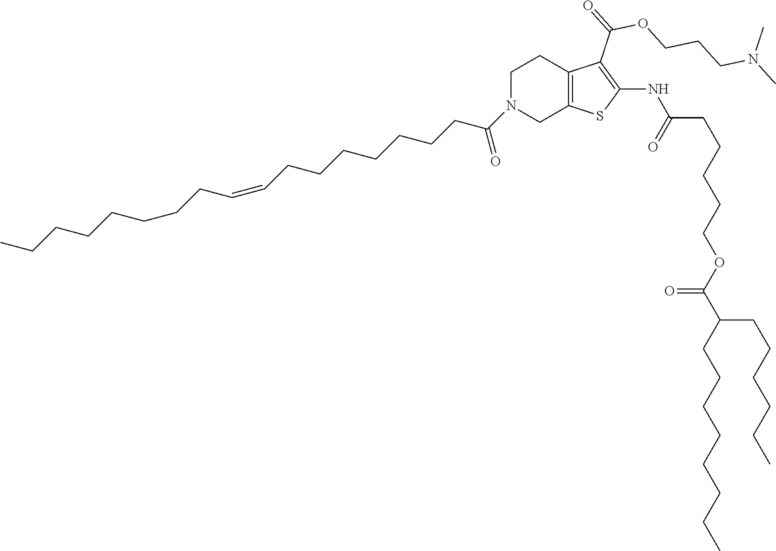 [SMILES: CCCCCCCC/C=C\CCCCCCCC(=O)N1CCc2c(sc(NC(=O)CCCCCOC(=O)C(CCCCCC)CCCCCCCC)c2C(=O)OCCCN(C)C)C1]

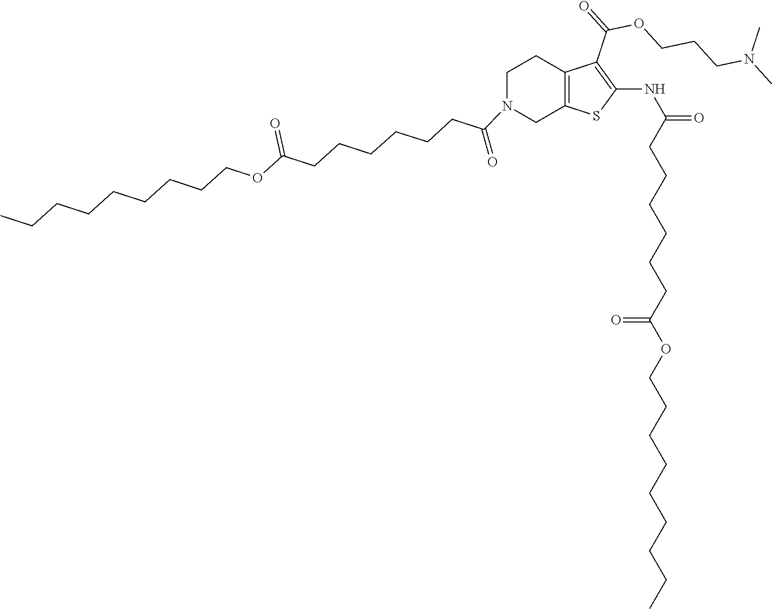 [SMILES: CCCCCCCCCOC(=O)CCCCCCC(=O)Nc1sc2c(c1C(=O)OCCCN(C)C)CCN(C(=O)CCCCCCC(=O)OCCCCCCCCC)C2]

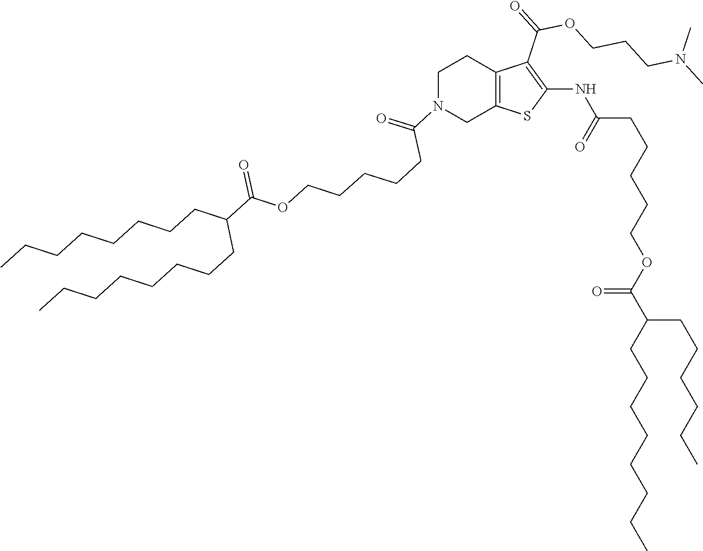 [SMILES: CCCCCCCCC(CCCCCC)C(=O)OCCCCCC(=O)Nc1sc2c(c1C(=O)OCCCN(C)C)CCN(C(=O)CCCCCOC(=O)C(CCCCCCCC)CCCCCCCC)C2]

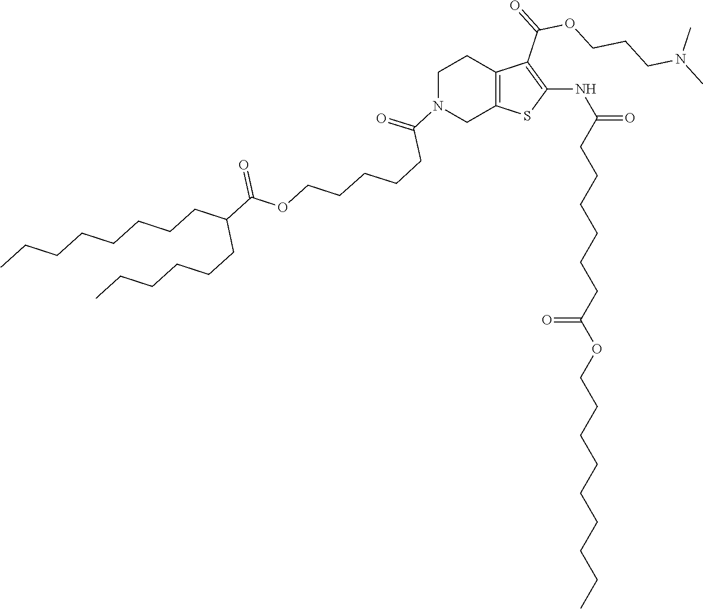 [SMILES: CCCCCCCCCOC(=O)CCCCCCC(=O)Nc1sc2c(c1C(=O)OCCCN(C)C)CCN(C(=O)CCCCCOC(=O)C(CCCCCC)CCCCCCCC)C2]

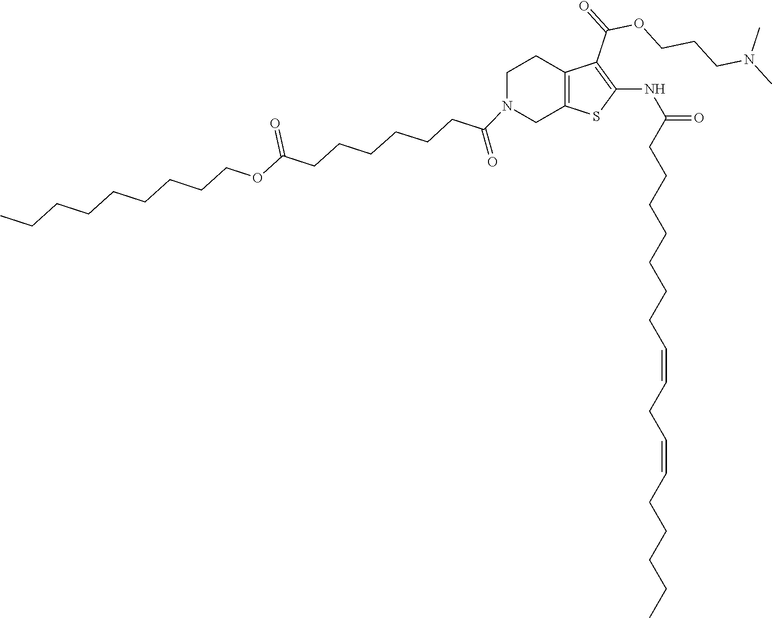 [SMILES: CCCCC/C=C\C/C=C\CCCCCCCC(=O)Nc1sc2c(c1C(=O)OCCCN(C)C)CCN(C(=O)CCCCCCC(=O)OCCCCCCCCC)C2]

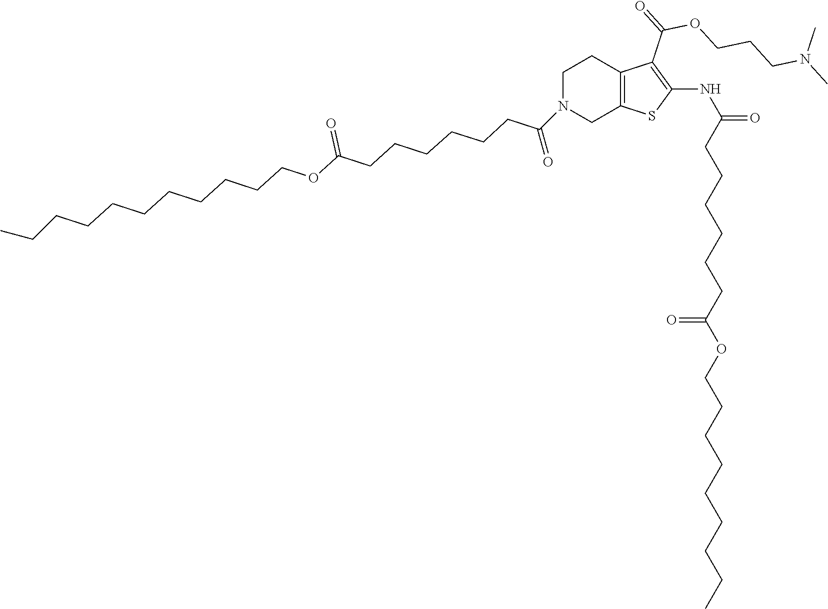 [SMILES: CCCCCCCCCCCOC(=O)CCCCCCC(=O)N1CCc2c(sc(NC(=O)CCCCCCC(=O)OCCCCCCCCC)c2C(=O)OCCCN(C)C)C1]

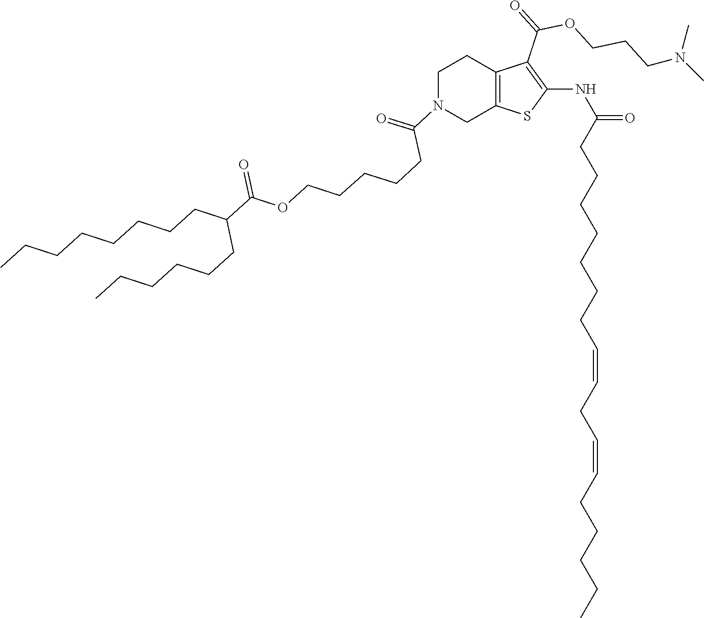 [SMILES: CCCCC/C=C\C/C=C\CCCCCCCC(=O)Nc1sc2c(c1C(=O)OCCCN(C)C)CCN(C(=O)CCCCCOC(=O)C(CCCCCC)CCCCCCCC)C2]

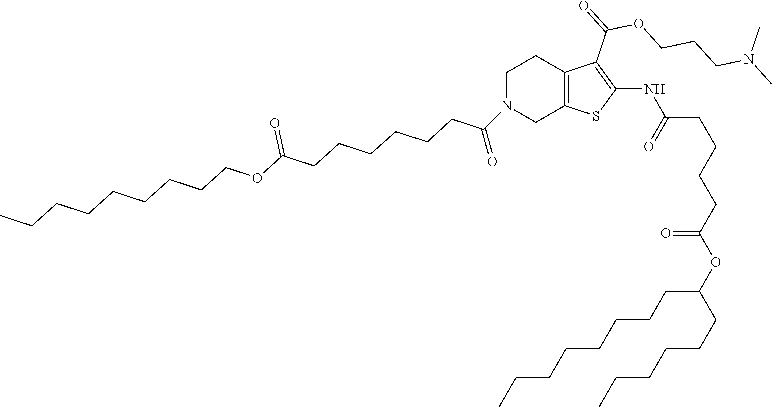 [SMILES: CCCCCCCCCOC(=O)CCCCCCC(=O)N1CCc2c(sc(NC(=O)CCCCC(=O)OC(CCCCCC)CCCCCCCC)c2C(=O)OCCCN(C)C)C1]

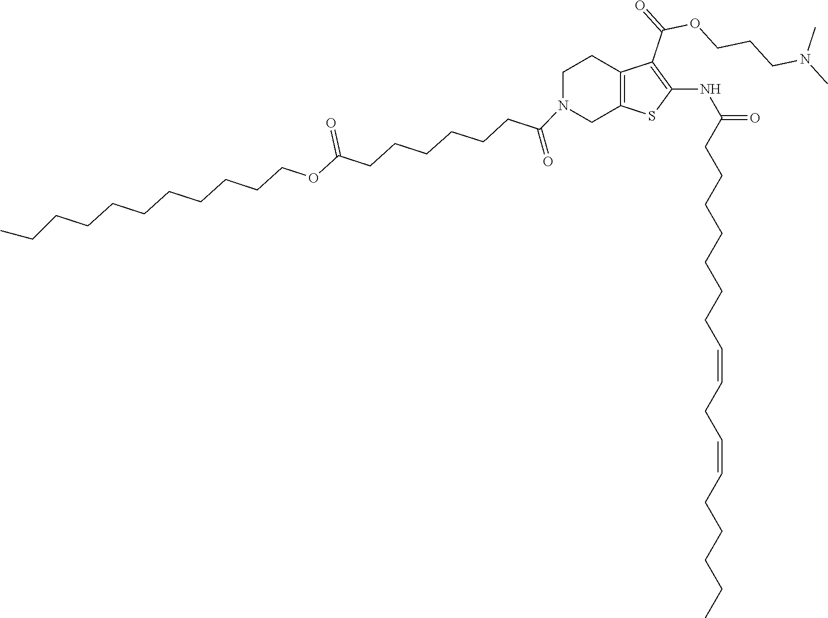 [SMILES: CCCCC/C=C\C/C=C\CCCCCCCC(=O)Nc1sc2c(c1C(=O)OCCCN(C)C)CCN(C(=O)CCCCCCC(=O)OCCCCCCCCCCC)C2]

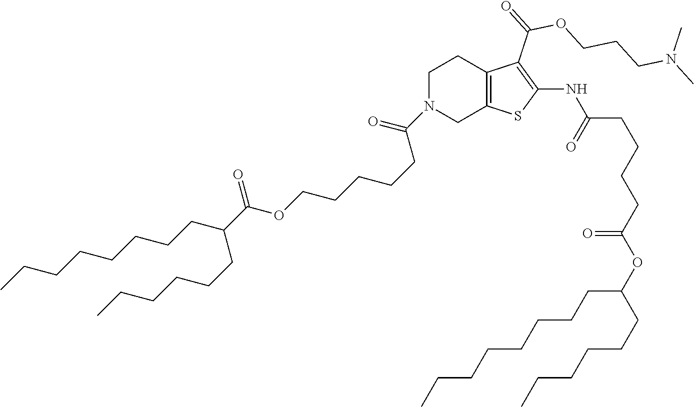 [SMILES: CCCCCCCCC(CCCCCC)OC(=O)CCCCC(=O)Nc1sc2c(c1C(=O)OCCCN(C)C)CCN(C(=O)CCCCCOC(=O)C(CCCCCC)CCCCCCCC)C2]

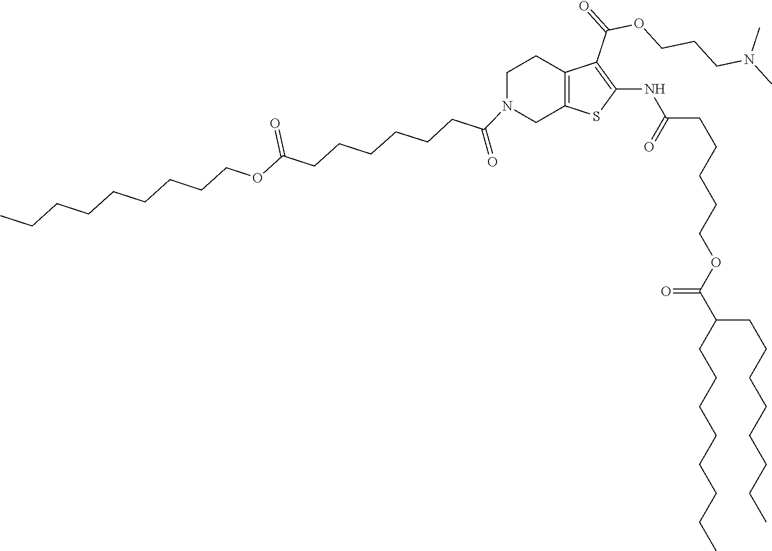 [SMILES: CCCCCCCCCOC(=O)CCCCCCC(=O)N1CCc2c(sc(NC(=O)CCCCCOC(=O)C(CCCCCCCC)CCCCCCCC)c2C(=O)OCCCN(C)C)C1]

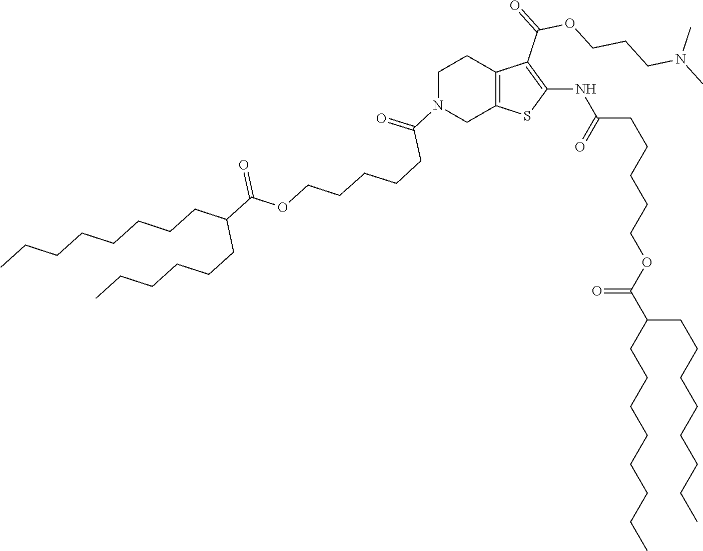 [SMILES: CCCCCCCCC(CCCCCC)C(=O)OCCCCCC(=O)N1CCc2c(sc(NC(=O)CCCCCOC(=O)C(CCCCCCCC)CCCCCCCC)c2C(=O)OCCCN(C)C)C1]

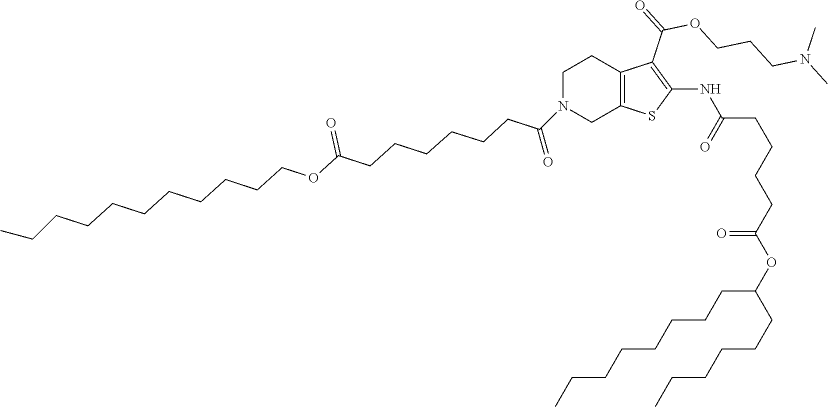 [SMILES: CCCCCCCCCCCOC(=O)CCCCCCC(=O)N1CCc2c(sc(NC(=O)CCCCC(=O)OC(CCCCCC)CCCCCCCC)c2C(=O)OCCCN(C)C)C1]

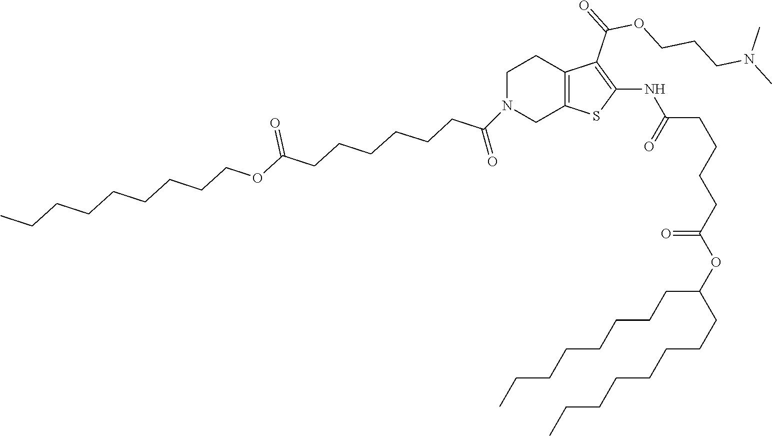 [SMILES: CCCCCCCCCOC(=O)CCCCCCC(=O)N1CCc2c(sc(NC(=O)CCCCC(=O)OC(CCCCCCCC)CCCCCCCC)c2C(=O)OCCCN(C)C)C1]

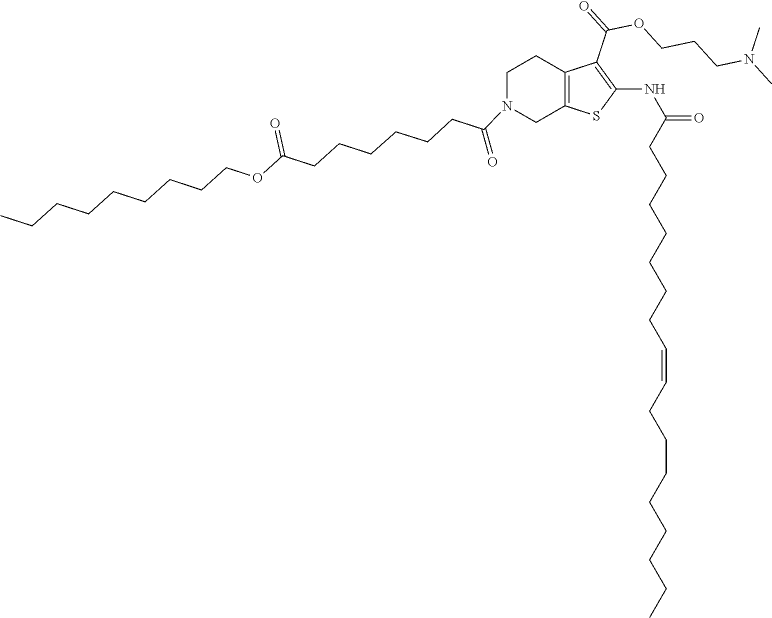 [SMILES: CCCCCCCC/C=C\CCCCCCCC(=O)Nc1sc2c(c1C(=O)OCCCN(C)C)CCN(C(=O)CCCCCCC(=O)OCCCCCCCCC)C2]

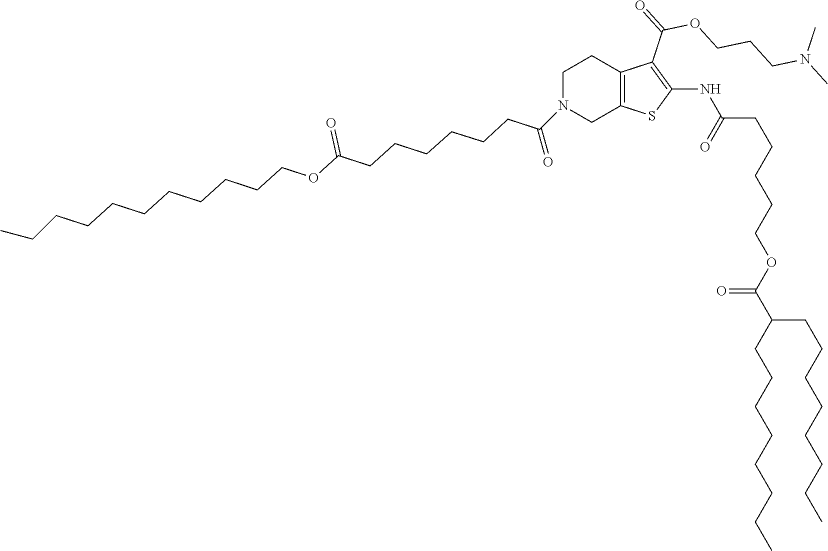 [SMILES: CCCCCCCCCCCOC(=O)CCCCCCC(=O)N1CCc2c(sc(NC(=O)CCCCCOC(=O)C(CCCCCCCC)CCCCCCCC)c2C(=O)OCCCN(C)C)C1]

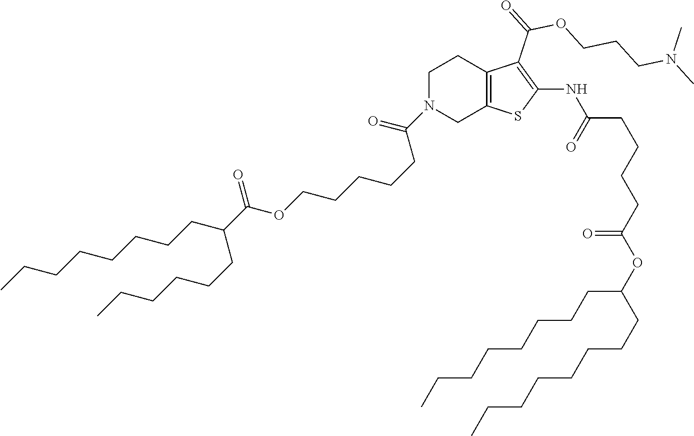 [SMILES: CCCCCCCCC(CCCCCCCC)OC(=O)CCCCC(=O)Nc1sc2c(c1C(=O)OCCCN(C)C)CCN(C(=O)CCCCCOC(=O)C(CCCCCC)CCCCCCCC)C2]

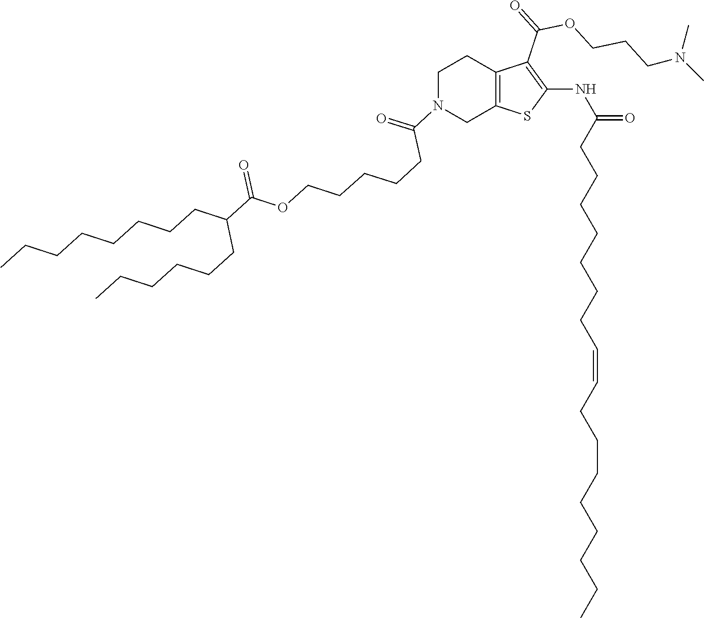 [SMILES: CCCCCCCC/C=C\CCCCCCCC(=O)Nc1sc2c(c1C(=O)OCCCN(C)C)CCN(C(=O)CCCCCOC(=O)C(CCCCCC)CCCCCCCC)C2]

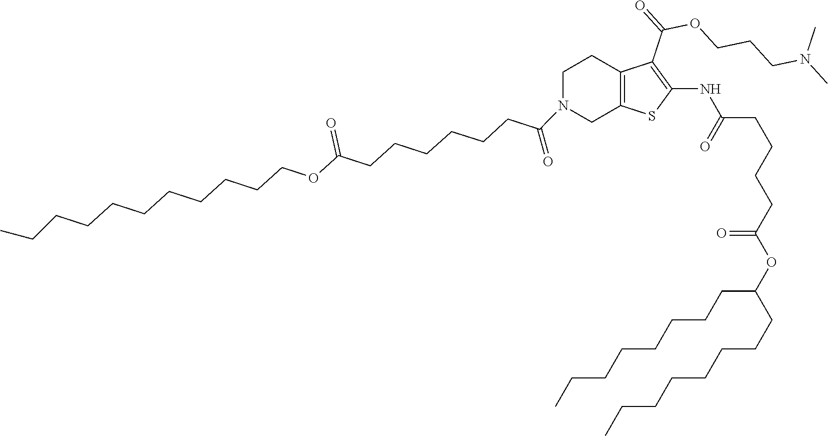 [SMILES: CCCCCCCCCCCOC(=O)CCCCCCC(=O)N1CCc2c(sc(NC(=O)CCCCC(=O)OC(CCCCCCCC)CCCCCCCC)c2C(=O)OCCCN(C)C)C1]

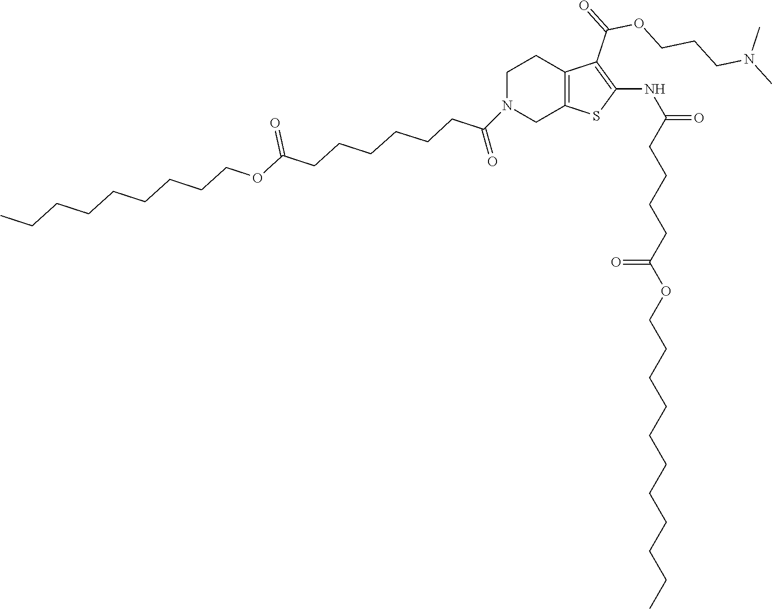 [SMILES: CCCCCCCCCCCOC(=O)CCCCC(=O)Nc1sc2c(c1C(=O)OCCCN(C)C)CCN(C(=O)CCCCCCC(=O)OCCCCCCCCC)C2]

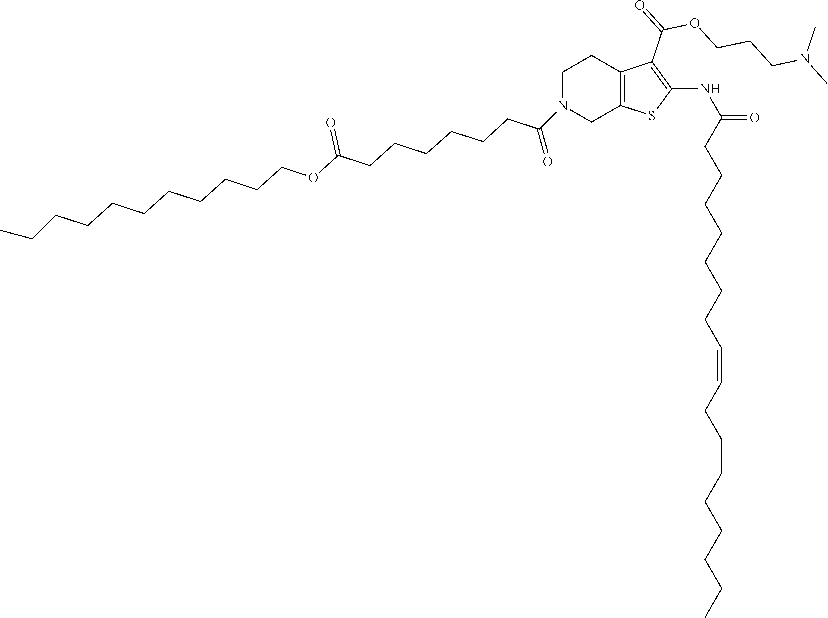 [SMILES: CCCCCCCC/C=C\CCCCCCCC(=O)Nc1sc2c(c1C(=O)OCCCN(C)C)CCN(C(=O)CCCCCCC(=O)OCCCCCCCCCCC)C2]

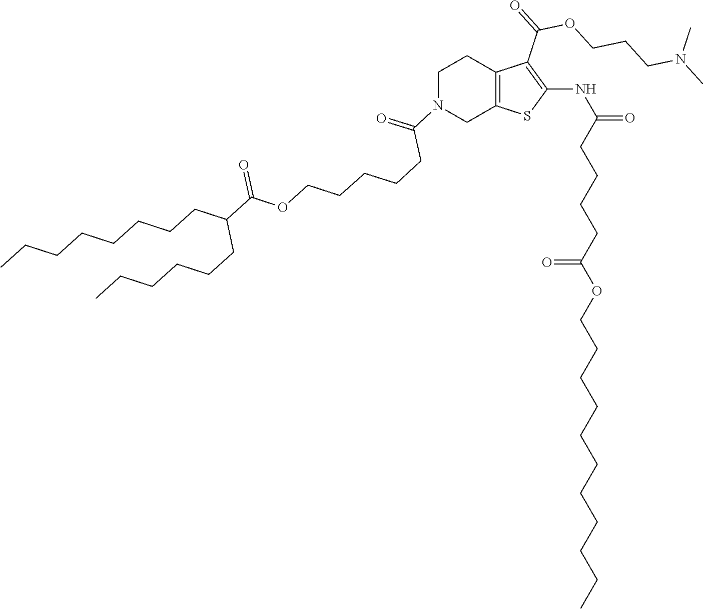 [SMILES: CCCCCCCCCCCOC(=O)CCCCC(=O)Nc1sc2c(c1C(=O)OCCCN(C)C)CCN(C(=O)CCCCCOC(=O)C(CCCCCC)CCCCCCCC)C2]

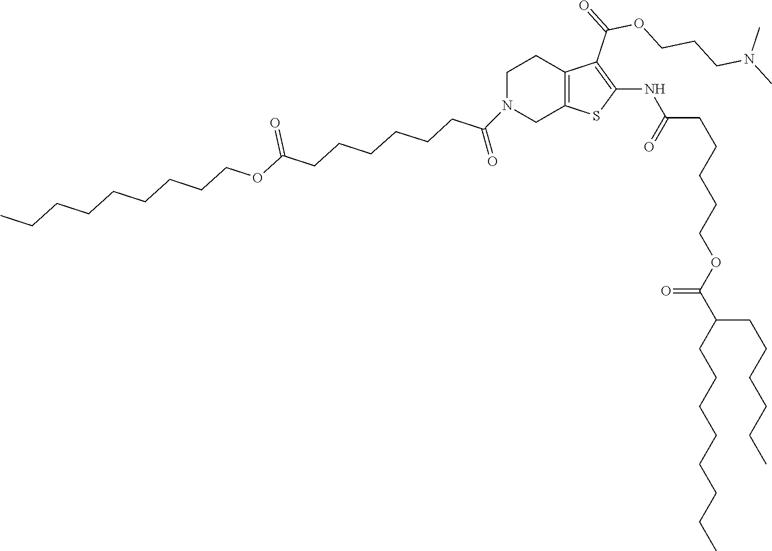 [SMILES: CCCCCCCCCOC(=O)CCCCCCC(=O)N1CCc2c(sc(NC(=O)CCCCCOC(=O)C(CCCCCC)CCCCCCCC)c2C(=O)OCCCN(C)C)C1]

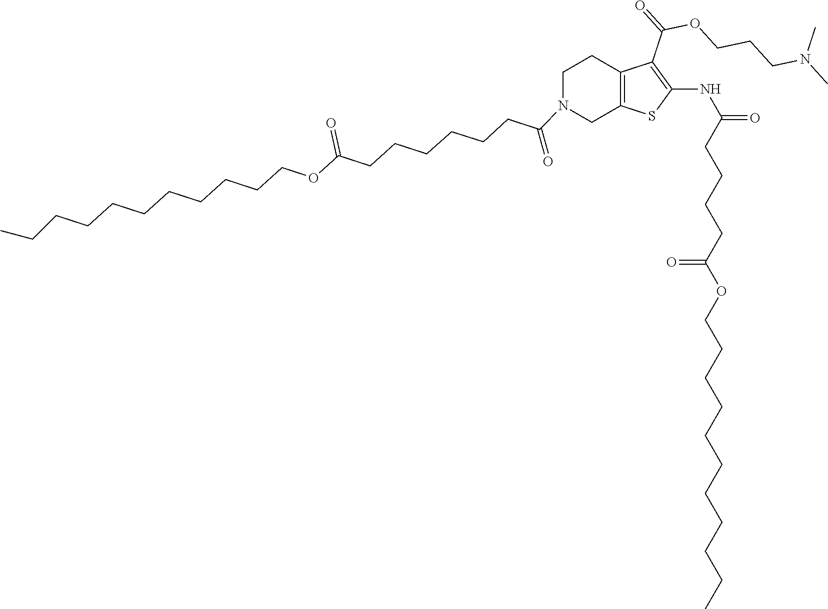 [SMILES: CCCCCCCCCCCOC(=O)CCCCCCC(=O)N1CCc2c(sc(NC(=O)CCCCC(=O)OCCCCCCCCCCC)c2C(=O)OCCCN(C)C)C1]

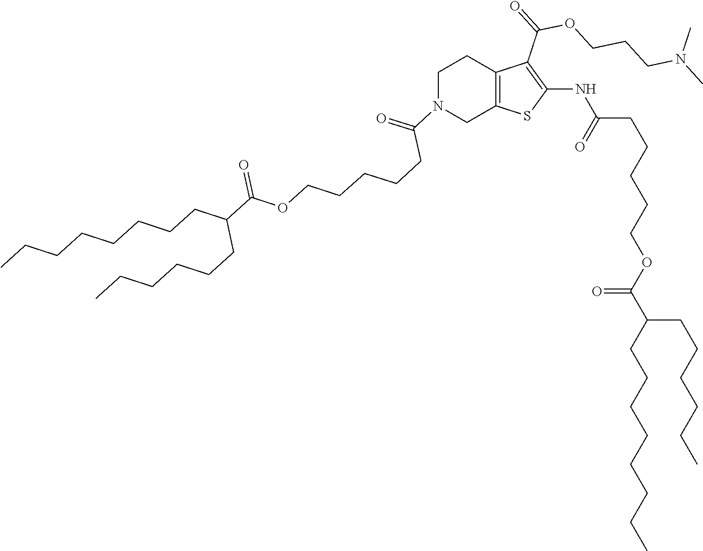 [SMILES: CCCCCCCCC(CCCCCC)C(=O)OCCCCCC(=O)Nc1sc2c(c1C(=O)OCCCN(C)C)CCN(C(=O)CCCCCOC(=O)C(CCCCCC)CCCCCCCC)C2]

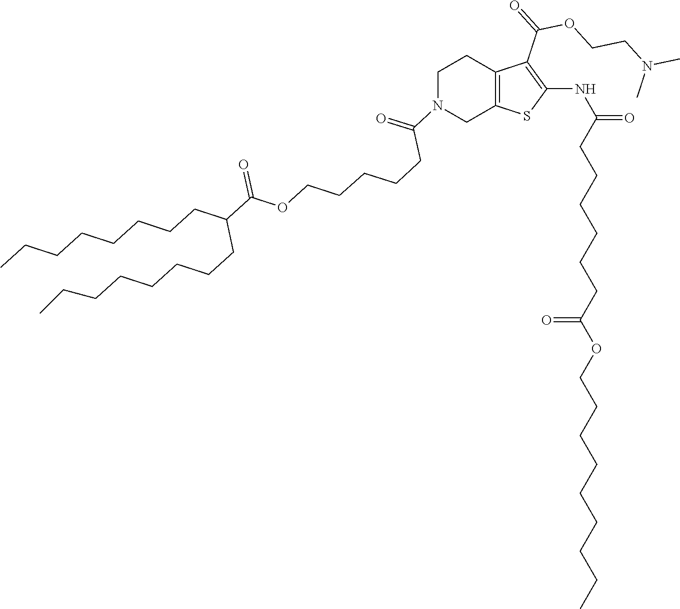 [SMILES: CCCCCCCCCOC(=O)CCCCCCC(=O)Nc1sc2c(c1C(=O)OCCN(C)C)CCN(C(=O)CCCCCOC(=O)C(CCCCCCCC)CCCCCCCC)C2]

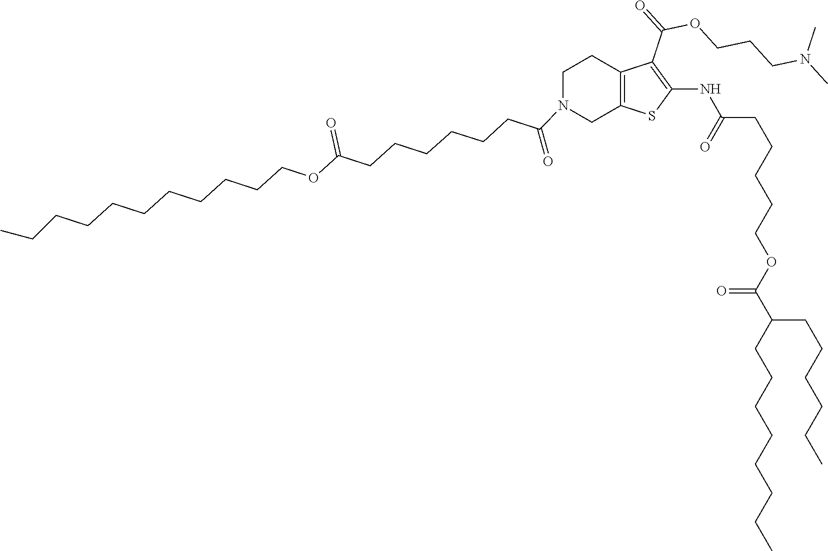 [SMILES: CCCCCCCCCCCOC(=O)CCCCCCC(=O)N1CCc2c(sc(NC(=O)CCCCCOC(=O)C(CCCCCC)CCCCCCCC)c2C(=O)OCCCN(C)C)C1]

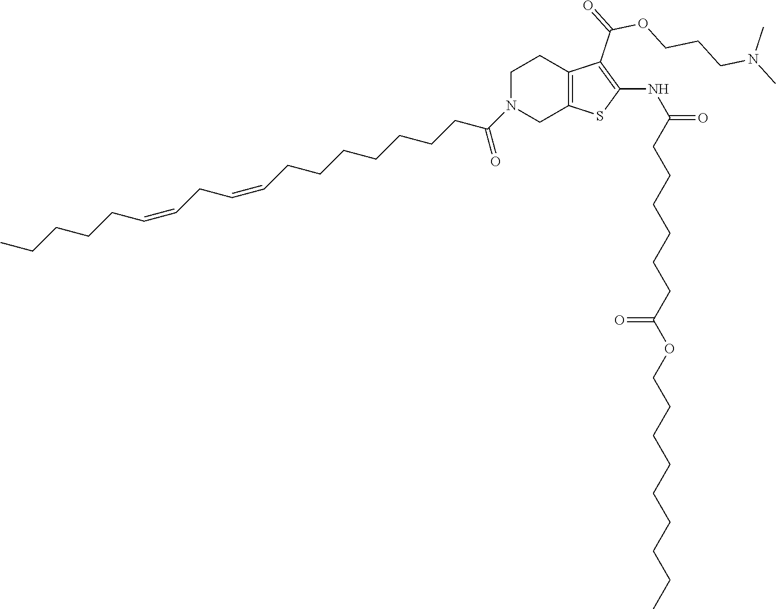 [SMILES: CCCCC/C=C\C/C=C\CCCCCCCC(=O)N1CCc2c(sc(NC(=O)CCCCCCC(=O)OCCCCCCCCC)c2C(=O)OCCCN(C)C)C1]

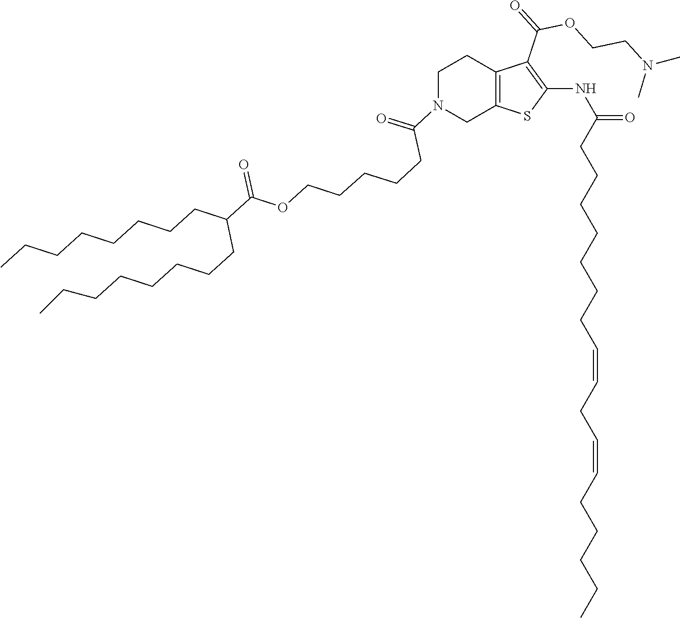 [SMILES: CCCCC/C=C\C/C=C\CCCCCCCC(=O)Nc1sc2c(c1C(=O)OCCN(C)C)CCN(C(=O)CCCCCOC(=O)C(CCCCCCCC)CCCCCCCC)C2]

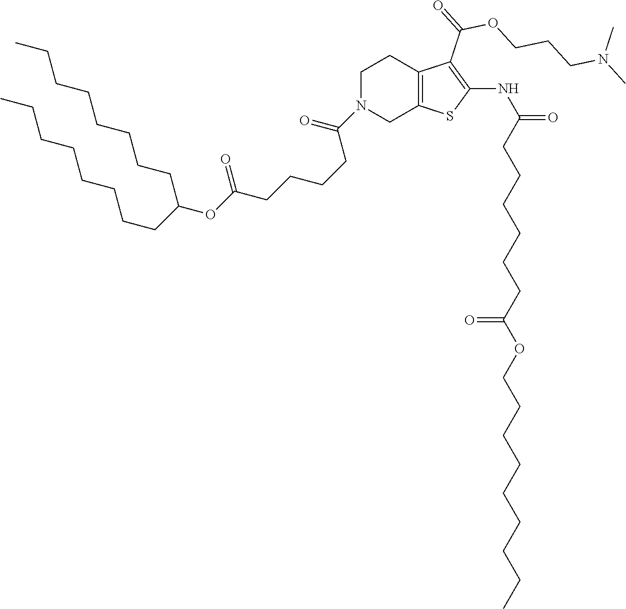 [SMILES: CCCCCCCCCOC(=O)CCCCCCC(=O)Nc1sc2c(c1C(=O)OCCCN(C)C)CCN(C(=O)CCCCC(=O)OC(CCCCCCCC)CCCCCCCC)C2]

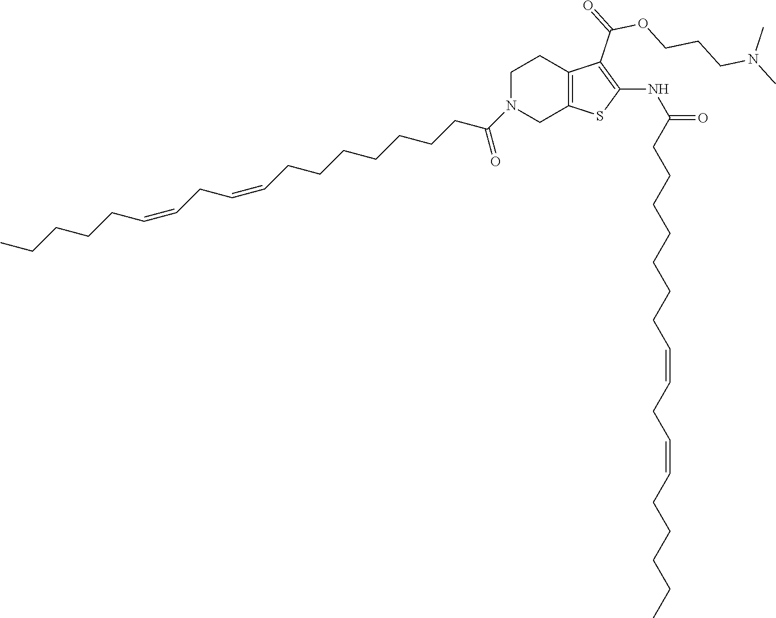 [SMILES: CCCCC/C=C\C/C=C\CCCCCCCC(=O)Nc1sc2c(c1C(=O)OCCCN(C)C)CCN(C(=O)CCCCCCC/C=C\C/C=C\CCCCC)C2]

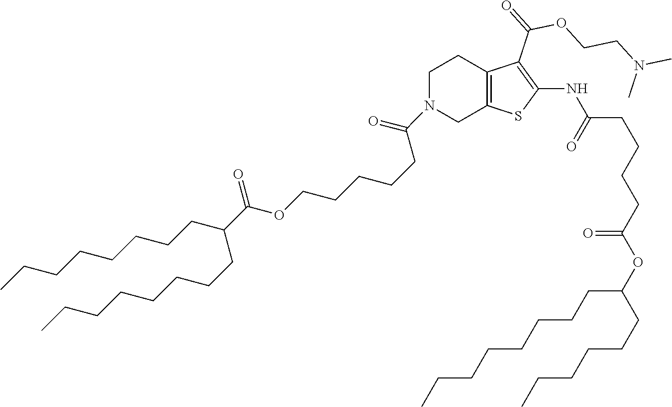 [SMILES: CCCCCCCCC(CCCCCC)OC(=O)CCCCC(=O)Nc1sc2c(c1C(=O)OCCN(C)C)CCN(C(=O)CCCCCOC(=O)C(CCCCCCCC)CCCCCCCC)C2]

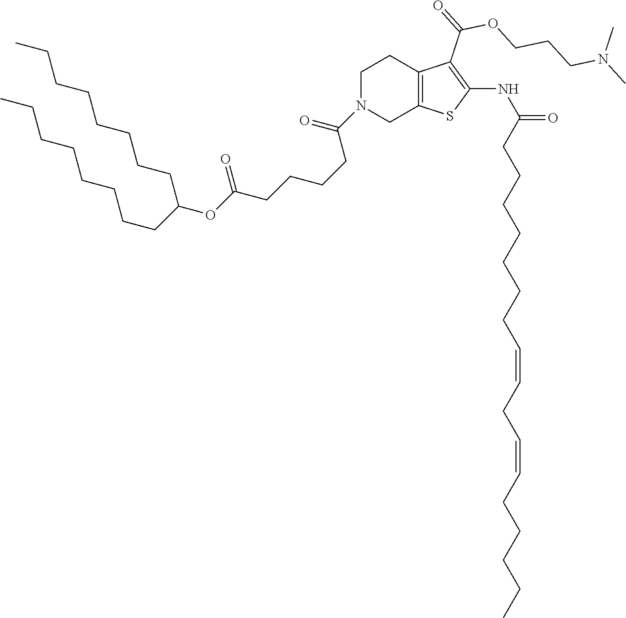 [SMILES: CCCCC/C=C\C/C=C\CCCCCCCC(=O)Nc1sc2c(c1C(=O)OCCCN(C)C)CCN(C(=O)CCCCC(=O)OC(CCCCCCCC)CCCCCCCC)C2]

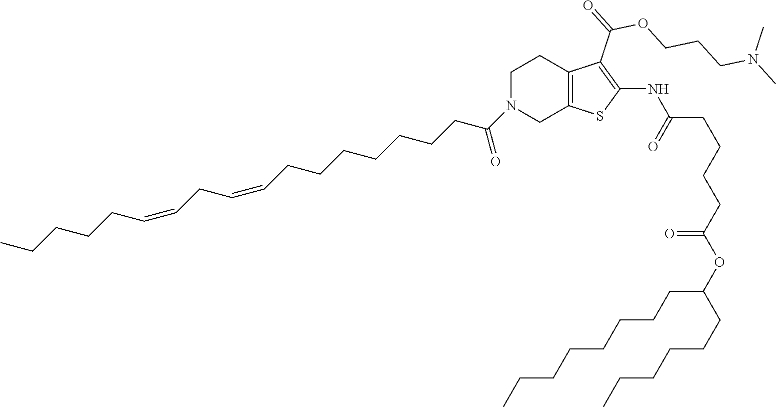 [SMILES: CCCCC/C=C\C/C=C\CCCCCCCC(=O)N1CCc2c(sc(NC(=O)CCCCC(=O)OC(CCCCCC)CCCCCCCC)c2C(=O)OCCCN(C)C)C1]

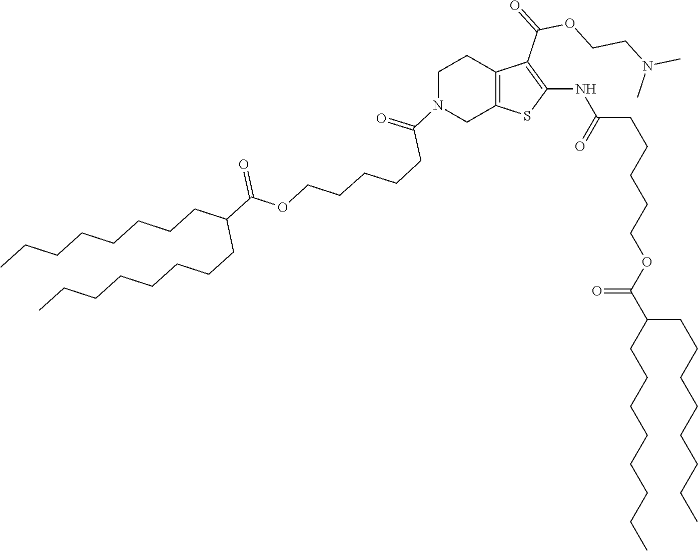 [SMILES: CCCCCCCCC(CCCCCCCC)C(=O)OCCCCCC(=O)Nc1sc2c(c1C(=O)OCCN(C)C)CCN(C(=O)CCCCCOC(=O)C(CCCCCCCC)CCCCCCCC)C2]

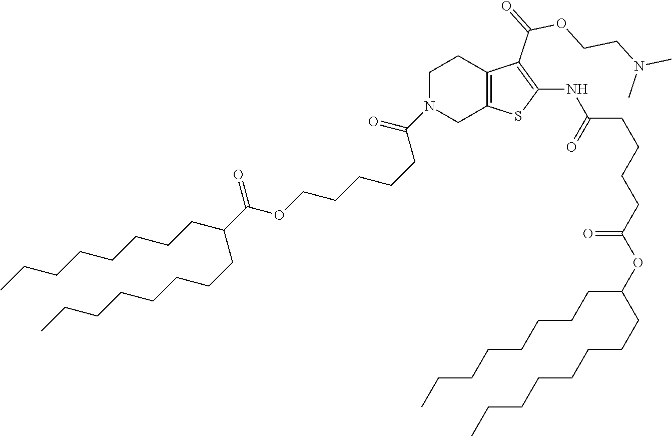 [SMILES: CCCCCCCCC(CCCCCCCC)OC(=O)CCCCC(=O)Nc1sc2c(c1C(=O)OCCN(C)C)CCN(C(=O)CCCCCOC(=O)C(CCCCCCCC)CCCCCCCC)C2]

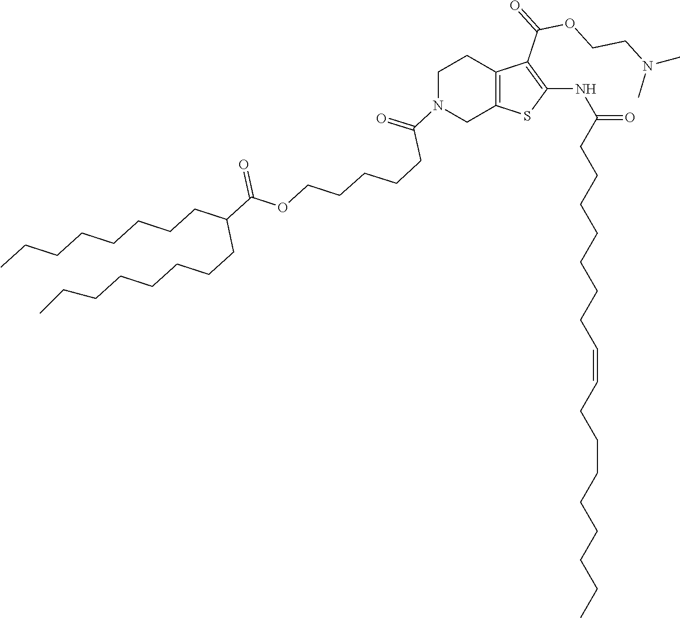 [SMILES: CCCCCCCC/C=C\CCCCCCCC(=O)Nc1sc2c(c1C(=O)OCCN(C)C)CCN(C(=O)CCCCCOC(=O)C(CCCCCCCC)CCCCCCCC)C2]

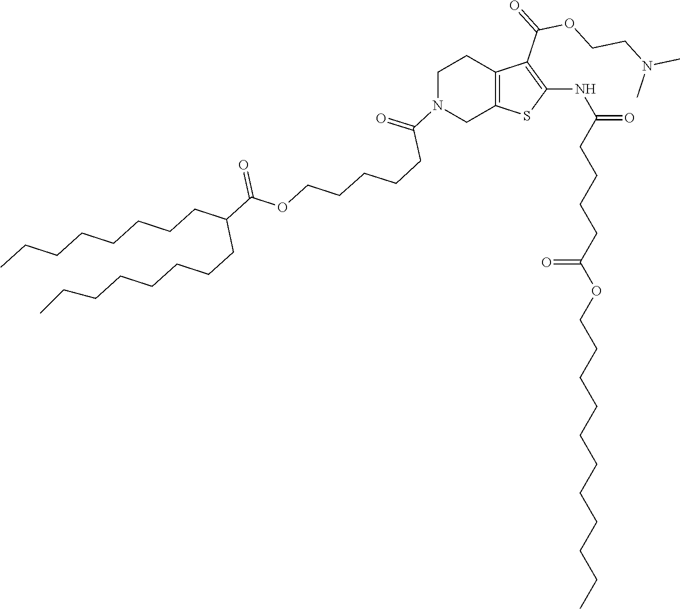 [SMILES: CCCCCCCCCCCOC(=O)CCCCC(=O)Nc1sc2c(c1C(=O)OCCN(C)C)CCN(C(=O)CCCCCOC(=O)C(CCCCCCCC)CCCCCCCC)C2]

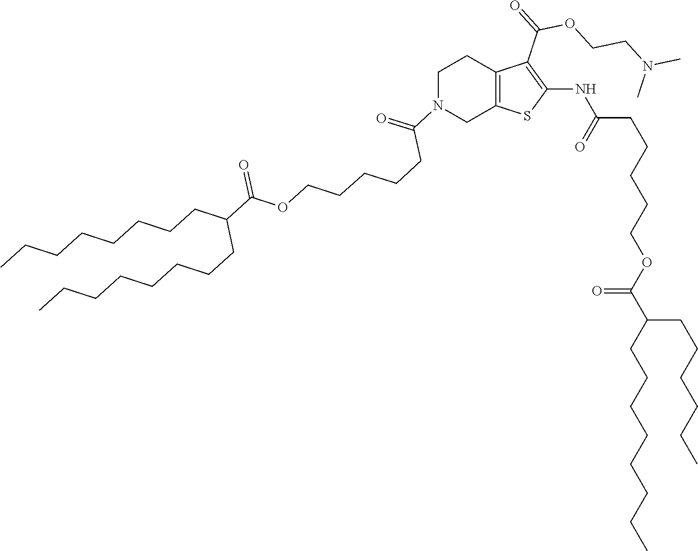 [SMILES: CCCCCCCCC(CCCCCC)C(=O)OCCCCCC(=O)Nc1sc2c(c1C(=O)OCCN(C)C)CCN(C(=O)CCCCCOC(=O)C(CCCCCCCC)CCCCCCCC)C2]

Further exemplary compounds include:

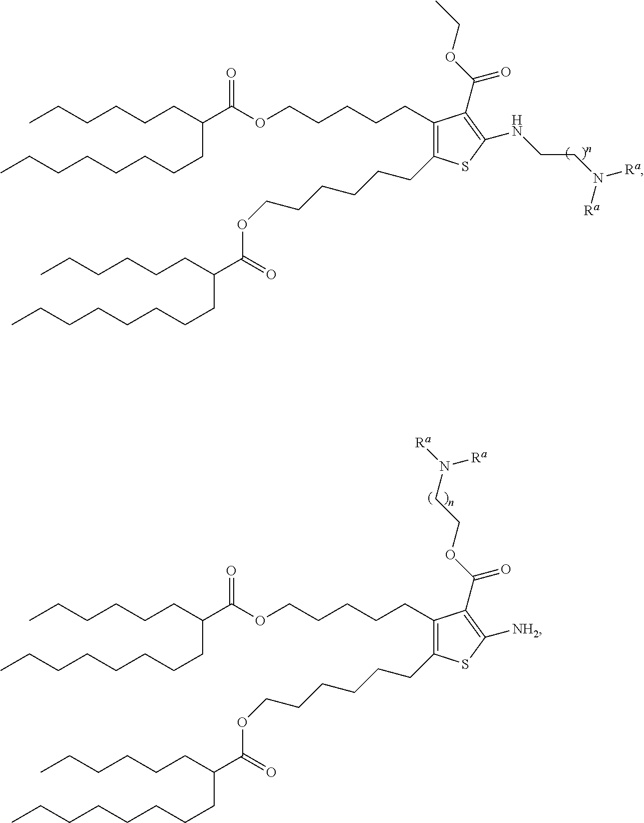

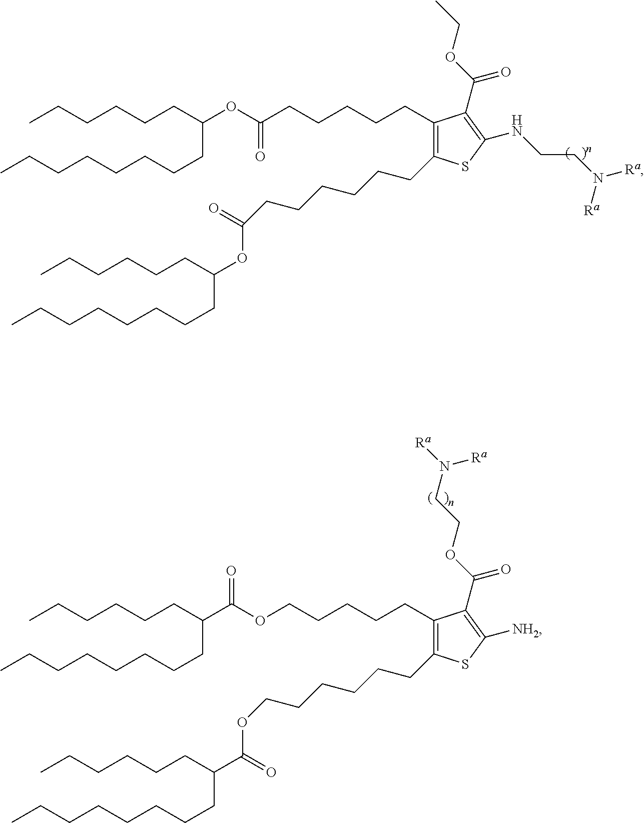

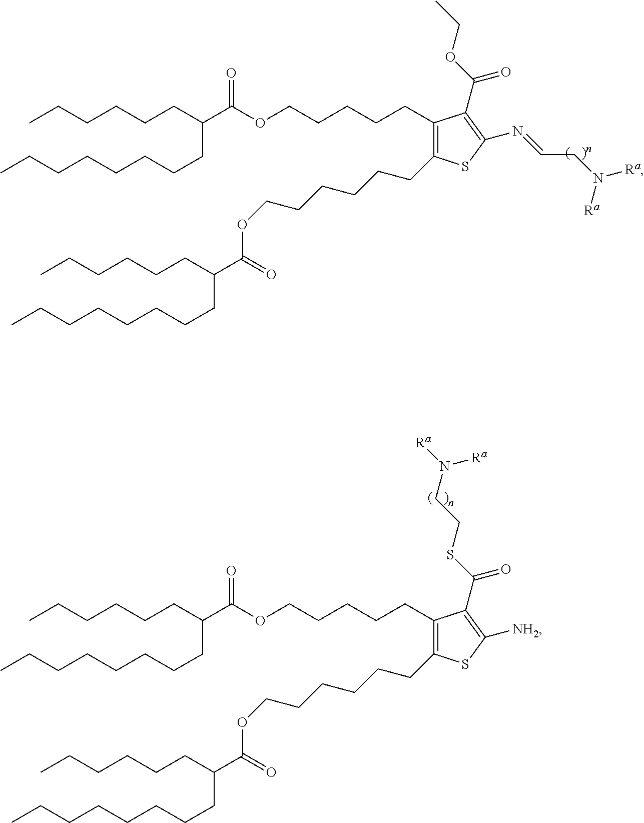

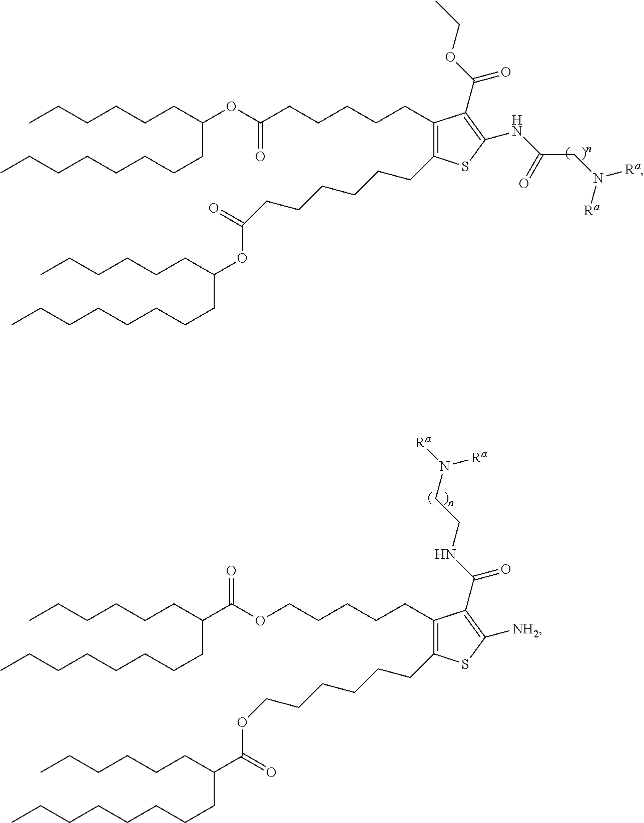

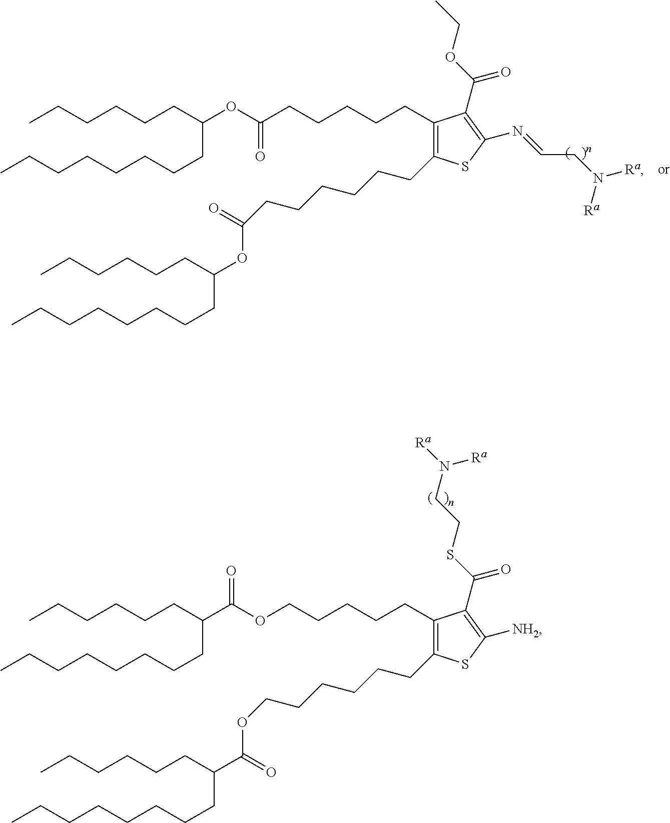

where n is from 0 to 6. and each R^{a} independently is as previously defined.

In certain embodiments, the compound is of the formula:

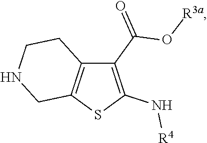

wherein R⁴ and R^{3a} are as shown in Table 1A:

TABLE 1A

| | R^{3a} | | | |
|---|---|---|---|---|
| R⁴ | 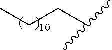 | 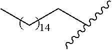 | 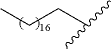 | 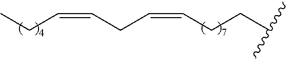 |
| 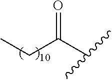 | 1-1 | 1-2 | 1-3 | 1-4 |
| 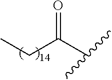 | 2-1 | 2-2 | 2-3 | 2-4 |
| 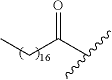 | 3-1 | 3-2 | 3-3 | |
| 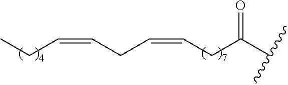 | 4-1 | 4-2 | 4-3 | |
| 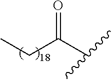 | 5-1 | 5-2 | 5-3 | 5-4 |
| 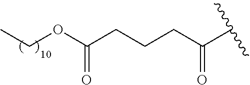 | 6-1 | 6-2 | 6-3 | 6-4 |
| 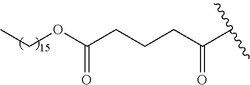 | 7-1 | 7-2 | 7-3 | 7-4 |
| 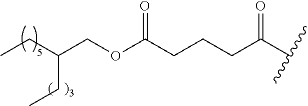 | 8-1 | 8-2 | 8-3 | 8-4 |
| 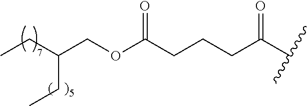 | 9-1 | 9-2 | 9-3 | 9-4 |
| 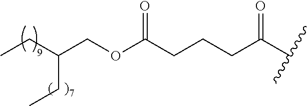 | 10-1 | 10-2 | 10-3 | 10-4 |
| 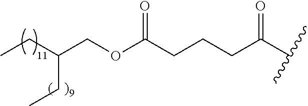 | 11-1 | 11-2 | 11-3 | 11-4 |

| | R^{3a} | | |
|---|---|---|---|
| R⁴ | 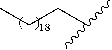 | 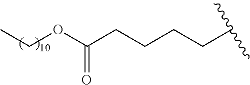 | 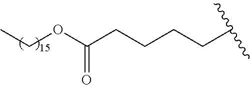 |
| 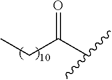 | 1-5 | 1-6 | 1-7 |
| 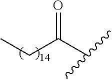 | 2-5 | 2-6 | 2-7 |
| 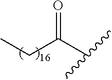 | 3-5 | 3-6 | 3-7 |
| 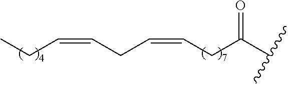 | 4-5 | 4-6 | 4-7 |
| 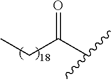 | 5-5 | 5-6 | 5-7 |
| 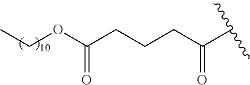 | 6-5 | 6-6 | 6-7 |
| 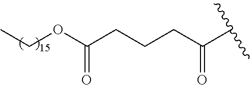 | 7-5 | 7-6 | 7-7 |
| 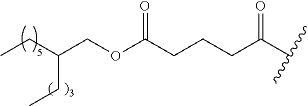 | 8-5 | 8-6 | 8-7 |
| 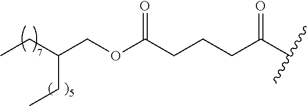 | 9-5 | 9-6 | 9-7 |
| 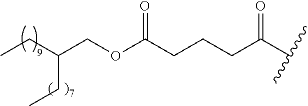 | 10-5 | 10-6 | 10-7 |
| 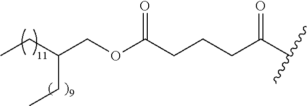 | 11-5 | 11-6 | 11-7 |

| | R^{3a} | |
|---|---|---|
| R⁴ | 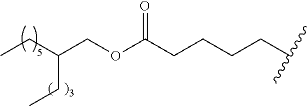 | 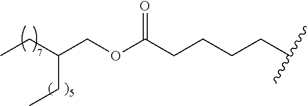 |
| 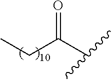 | 1-8 | 1-9 |
| 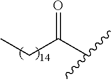 | 2-8 | 2-9 |
| 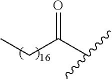 | 3-8 | 3-9 |
| 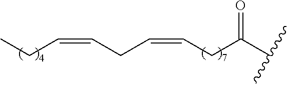 | 4-8 | 4-9 |
| 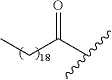 | 5-8 | 5-9 |
| 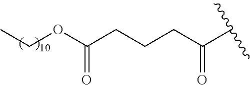 | 6-8 | 6-9 |
| 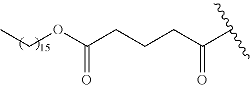 | 7-8 | 7-9 |
| 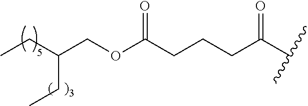 | 8-8 | 8-9 |
| 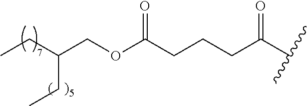 | 9-8 | 9-9 |
| 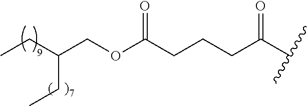 | 10-8 | 10-9 |
| 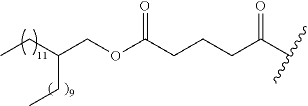 | 11-8 | 11-9 |

| | R^{3a} | |
|---|---|---|
| R⁴ | 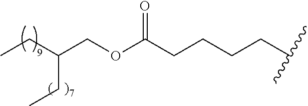 | 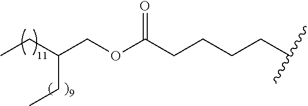 |
| 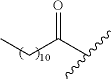 | 1-10 | 1-11 |
| 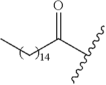 | 2-10 | 2-11 |
| 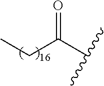 | 3-10 | 3-11 |
| 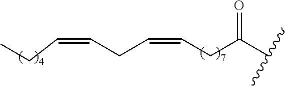 | 4-10 | 4-11 |
| 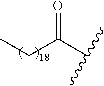 | 5-10 | 5-11 |
| 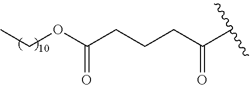 | 6-10 | 6-11 |
| 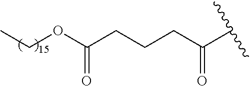 | 7-10 | 7-11 |
| 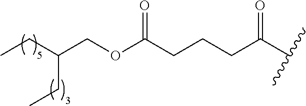 | 8-10 | 8-11 |
| 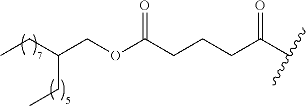 | 9-10 | 9-11 |
| 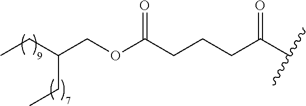 | 10-10 | 10-11 |
| 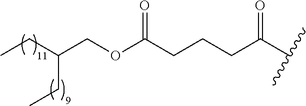 | 11-10 | 11-11 |

In certain embodiments, the compound is of the formula:

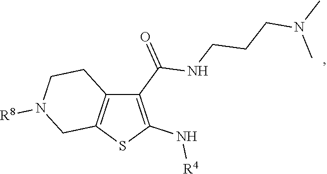

wherein R⁴ and R⁸ are as shown in Table 1B:

TABLE 1B

| | R⁴ | | | |
|---|---|---|---|---|
| R⁸ | 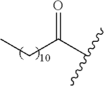 | 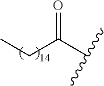 | 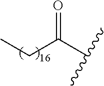 | 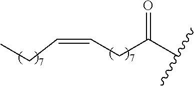 |
| 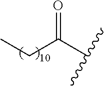 | 12-12 | 12-13 | 12-14 | 12-15 |
| 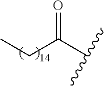 | 13-12 | 13-13 | 13-14 | 13-15 |
| 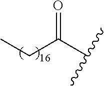 | 14-12 | 14-13 | | 14-15 |
| 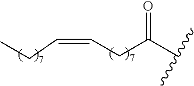 | 15-12 | 15-13 | 15-14 | |
| 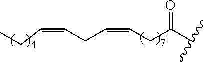 | 16-12 | 16-13 | 16-14 | |
| 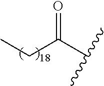 | 17-12 | 17-13 | 17-14 | 17-15 |
| 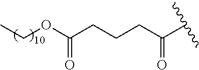 | 18-12 | 18-13 | 18-14 | 18-15 |
| 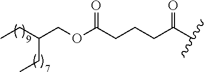 | 19-12 | 19-13 | 19-14 | 19-15 |
| 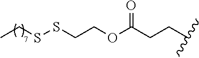 | 20-12 | 20-13 | 20-14 | 20-15 |

| | R⁴ | | |
|---|---|---|---|
| R⁸ | 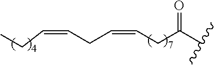 | 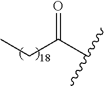 | 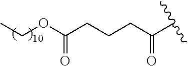 |
| 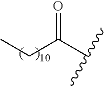 | 12-16 | 12-17 | 12-18 |
| 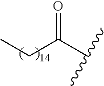 | 13-16 | 13-17 | 13-18 |
| 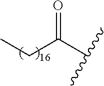 | 14-16 | 14-17 | 14-18 |
| 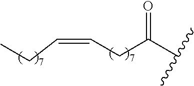 | | 15-17 | 15-18 |
| 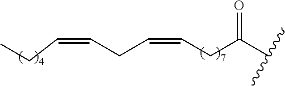 | | 16-17 | 16-18 |
| 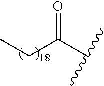 | 17-16 | 17-17 | 17-18 |
| 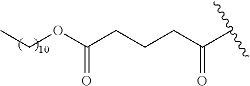 | 18-16 | 18-17 | 18-18 |
| 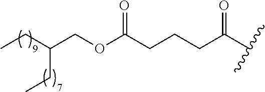 | 19-16 | 19-17 | 19-18 |
| 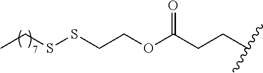 | 20-16 | 20-17 | 20-18 |

| | R⁴ | |
|---|---|---|
| R⁸ | 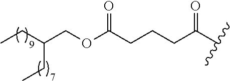 | 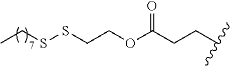 |
| 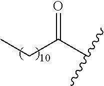 | 12-19 | 12-20 |
| 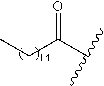 | 13-19 | 13-20 |
| 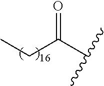 | 14-19 | 14-20 |
| 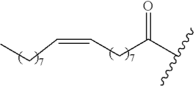 | 15-19 | 15-20 |
| 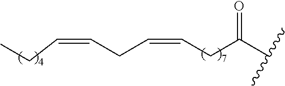 | 16-19 | 16-20 |
| 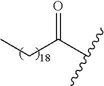 | 17-19 | 17-20 |
| 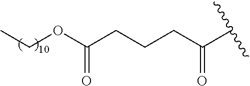 | 18-19 | 18-20 |
| 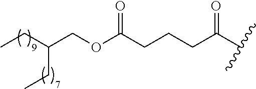 | 19-19 | 19-20 |
| 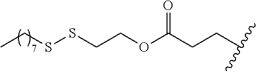 | 20-19 | 20-20. |

In Tables 1A and 1B, the numbers in the format “XX-YY,” where XX and YY are integers, refer to formula numbers. For example, “1-1” refers to Formula 1-1.

In certain embodiments, the compound is of the formula:

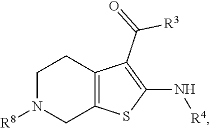

wherein R⁴ and R⁸ are as shown in Table 1B, and R³ is of the formula:

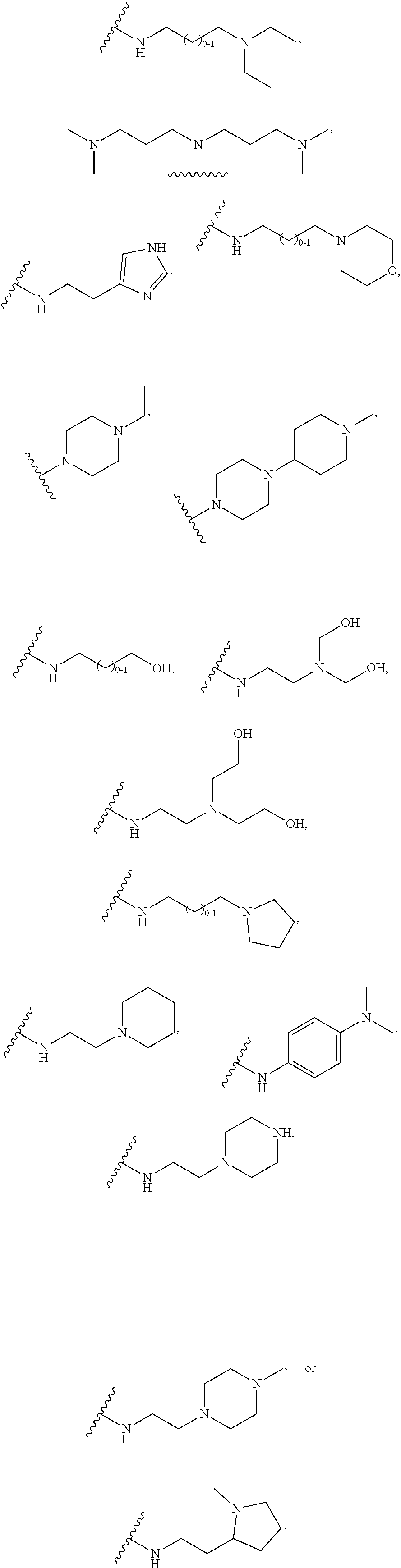

### III. Nanoparticle and Microparticle Preparation

Lipid nanoparticle (LNP) compositions may include one or more lipid components and one or more agents, such as a nucleic acid molecule, that may be associated and/or encapsulated by the lipid components. Lipid microparticle compositions may include one or more lipid components and one or more agents, such as a nucleic acid molecule, that may be associated and/or encapsulated by the lipid components. A nanoparticle composition may be designed for one or more specific applications, targets, and/or diseases. The elements of a nanoparticle composition may be selected based on a particular application or target and/or based on the efficacy, toxicity, expense, ease of use, availability, synthetic pathways, or other properties. In some embodiments, the lipid nanoparticles comprise five components: 1) nucleic acid (e.g., mRNA), 2) ionizable lipid (e.g., a lipid disclosed herein), 3) phospholipid (e.g., 1,2-distearoyl-sn-glycero-3-phosphocholine (DSPC)), 4) structural lipid (e.g., cholesterol) and 5) polymer-conjugated lipid (e.g. DMG-PEG-2000). The chemical structures and quantities of comprising lipids and other constituents may influence gene delivery efficacy, particle stability, and toxicity to the cells, and may be adjusted depending on the applications or targets. In general, the quantities of the lipids may be described by the molar ratios between the comprising components.

### A. Preparation and Properties

Various mixing methods and protocols may be used to produce the formulations. Specifically, lipid nanoparticles can be made by any suitable mixing processes such as, but not limited to, pipet mixing, mixing by a liquid handling system, syringe mixing, T-junction mixing, or microfluidic mixing of two or more solutions and/or suspensions, one of which contains one or more nucleic acids and the other has lipid components. The lipids nanoparticles may also be prepared by mixing method such as, but not limited to, pipet mixing, mixing by a liquid handling system, syringe mixing, T-junction mixing, or microfluidic mixing of two or more solutions and/or suspensions, one of which contains lipid nanoparticles prepared using aforementioned methods without nucleic acids or with one or more nucleic acids, and the other contains one or more nucleic acids. Additional chemical compounds may be introduced in either or both of the fluid volumes or streams depending on their solubility. In certain embodiments, the nucleic acid solution includes an aqueous buffer, such as a citrate or acetate buffer to maintain the solution at acidic pH, such as at a pH of from less than 7, for example, 3, 4 5, or 6. In certain embodiments, the lipids are dissolved in alcohol, such as ethanol. In certain embodiments, the lipid nanoparticles are suspended in an aqueous buffer, such as phosphate-buffered saline, Tris buffer, HEPES buffer to maintain the solution at neutral pH, for example, 7 or 7.5, or citrate or acetate buffer to maintain the solution at acidic pH, such as at a pH of from less than 7, for example, 3, 4 5, or 6.

The volumes of the fluids, the flow rate of the fluid streams, may be adjusted or optimized depending on the amount of nucleic acids, target nanoparticle size, polydispersity, encapsulation efficiency, and other features. A volumetric ratio between nucleic acid solution and lipid solution may vary from about 1:1 to 1:8, such as 1:2, 1:3, 1:4, 1:5, 1:6, 1:7, and 1:8. In some embodiments, the total flow rate may be from about 8 ml/min to 20 ml/min.

In certain embodiments, after addition, the mixture is isolated, or purified from unincorporated components. The mixture may be dialyzed against, or washed with a physiological buffer, for example, phosphate buffered saline, or used without further purification. After the purification is complete, the nanoparticles may be concentrated, for example, in a centrifuge.

In certain embodiments, the mean size of a nanoparticle composition may be from 10's of nm to 100's of nm. A nanoparticle composition may be relatively homogenous as indicated by a polydispersity index which may vary from 0 to 1. A small (e.g., less than 0.3) polydispersity index may indicate a narrow particle size distribution. Zeta potential of a nanoparticle composition may be used to indicate electrokinetic potentials of the composition. For example, the zeta potential may describe the surface charge of a nanoparticle composition. The zeta potential of a nanoparticle composition may be from about −40 mV to about +40 mV.

### B. Composition

In certain embodiments, the composition comprises a compound provided herein. In certain embodiments, the composition comprises an excipient. In certain embodiments, the excipient is a pharmaceutically acceptable excipient. In certain embodiments, the lipid nanoparticles comprise up to five components or more, including: 1) nucleic acid, 2) ionizable lipid, 3) phospholipid, 4) structural lipid, and 5) polymer-conjugated lipid. However, a lipid nanoparticle may contain as few as 2 components out of those listed above (e.g. ionizable lipid and nucleic acid). In addition, a lipid nanoparticle may have more than one of each constituent (e.g., additional nucleic acid, ionizable, structural and/or PEG lipids, and/or phospholipid).

The amount of thiophene-based ionizable lipid, such as a lipid disclosed herein, may be from 0.1 to 100 mol % of the total amount of lipids in the nanoparticle, such as from 0.1 to 0.3 mol %, from 0.3 to 1 mol %, from 1 to 3 mol %, from 3 to 10 mol %, from 10 to 30 mol %, from 30 to 50 mol %, from 50 to 70 mol %, from 70 to 90 mol %, or from 90 to 100 mol %, of the total amount of lipids in the nanoparticle. The amount of thiophene-based ionizable lipid, such as a lipid disclosed herein, may be from 20 to 100 mol % of the total amount of lipids in the nanoparticle, such as from 20 to 90 mol %, from 20 to 80 mol % or from 20 to 70 mol % of the total amount of lipids in the nanoparticle.

The amount of phospholipid may be from 0 to 30 mol % of the total lipids, such as from greater than zero to 30 mol %. The phospholipid may be any phospholipid suitable to form the nanoparticles, such as, but not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE).

The amount of structural lipid may be from 0 to 60 mol % of the total amount of lipids, such as from greater than zero to 60 mol %. The structural lipid may be any suitable structural lipid, such as, but not limited to, cholesterol, beta-sitosterol, fucosterol, campesterol, or stigmastanol.

The amount of polymer-conjugated lipid (PEG-lipid) may be from 0 to 10 mol % of the total amount of lipids, such as from greater than zero to 10 mol %. The PEG lipid may be any suitable PEG lipid, such as, but not limited to, PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. In certain embodiments, the polymer-conjugated lipid is (distearoyl-phosphatidyl-ethanolamine)-PEG-C(O)OH or (distearoyl-phosphatidyl-ethanolamine)-PEG-carboxy-NHS. For example, a PEG lipid may be PEG-c-DOMG, PEG-DMG, PEG-DLPE, PEG-DMPE, PEG-DPPC, a PEG-DSPE lipid or PEG-lipid derivatives, such as, but not limited to that contain carboxylic acid or amine at the PEG terminal. In any embodiments, the PEG chain may have a molecular weight of from about 1000 daltons to about 20,000 daltons or more. PEG chain lengths of commercially available PEG compounds may be already determined by the vendor. And/or the length may be determined by a suitable technique, such as GPC (gel permeation chromatography).

Pharmaceutical compositions can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

In some embodiments, pharmaceutical compositions are prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, in some embodiments, delayed absorption of a parenterally administered drug form is accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares. Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

In some embodiments, the compounds and compositions provided herein are administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound, mode of administration, and the like. In some embodiments, an effective amount is included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. In some embodiments, the amount to be administered to, for example, a child or an adolescent is determined by a medical practitioner or person skilled in the art. In some embodiments, the amount to be administered to, for example, a child or an adolescent is lower or the same as that administered to an adult.

In some embodiments, a compound or composition, as described herein, is administered in combination with one or more additional pharmaceutical agents (e.g., therapeutically and/or prophylactically active agents). In some embodiments, the compounds or compositions are administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, improve bioavailability, improve safety, reduce drug resistance, reduce and/or modify metabolism, inhibit excretion, and/or modify distribution in a subject, cell, tissue, or biological sample. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

In some embodiments, the compound or composition is administered concurrently with, prior to, or subsequent to one or more additional pharmaceutical agents. In some embodiments, the one or more additional pharmaceutical agents are useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease. In some embodiments, each additional pharmaceutical agent is administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the additional pharmaceutical agent is a leukotriene inhibitor. In certain embodiments, the additional pharmaceutical agent is an anti-inflammatory agent. In certain embodiments, the additional pharmaceutical agent is an immunosuppressant. In certain embodiments, the additional pharmaceutical agent is an immunostimulant. In certain embodiments, the compounds or pharmaceutical compositions described herein are administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the compound of the present disclosure is useful in delivering the additional pharmaceutical agent (e.g., to a subject, cell, tissue, or biological sample).

### C. Nucleic Acid Cargo

The nucleic acid being present in the lipid nanoparticle of the present invention includes known any type of nucleic acid. The nucleic acid may be described as a therapeutic and/or prophylactic nucleic acid. Nucleic acid can be any single stranded DNA or RNA, either double-stranded DNA or the RNA-RNA or DNA-RNA hybrids; shortmers, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and mixtures thereof. In certain embodiments, the nucleic acid is a plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, viral DNA, circular RNA (circRNA), precursor messenger RNA (pre-mRNA), microRNA (miRNA), guide RNA (gRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, viral satellite RNA, or a combination thereof.

Double-stranded DNA may include, but are not limited to, genes of interest for protein production (antigen protein), genome editing component (such as Cas9 and prime editors), and mobile genetic elements. Double-stranded RNA may include, but are not limited to, small-interfering RNA (siRNA) and other RNA-interference (RNAi) molecules. And single-stranded nucleic acids include, but are not limited to, messenger RNA, antisense oligonucleotides (ASO), and microRNA (miRNA).

Examples of messenger RNA suitable for use in the disclosed lipid nanoparticles include, but are not limited to, Firefly luciferase (Fluc), Nanoluciferase (Nluc), Green Fluorescent Protein (GFP), Cre recombinase, Transposase, Cas9 endonuclease, Cas13 endonuclease, Prime editor, Base editor, Spike protein of SARS-CoV-2, Human soluble angiotensin-converting enzyme 2 (ACE2), Human erythropoietin (EPO), Human alpha-galactosidase, Human Factor IX (FIX), Human Factor XI (FXI), Human cystic fibrosis transmembrane conductance regulator (CFTR), Human epithelial sodium channel (ENaC), Human interleukins (ILs), Human transcription factor EB (TFEB), or a combination thereof.

The nucleic acid optionally may have one or more modifications that confer stability to the nucleic acid (e.g., compared to a wild-type or native version of the nucleic acid), one or more modification that reduce side-effect of nucleic acid, and/or may also comprise one or more modifications relative to the wild-type which correct a defect implicated in the disease-associated, aberrant expression of the protein.

The molar ratio between ionizable lipid and nucleic acid may vary from about 1:1 to about 30:1, such as 1:1, 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. This ratio may include every charged group in a molecule. Similarly, the wt/wt ratio of total lipid component to a therapeutic and/or prophylactic nucleic acid may be from about 2:1 to about 60:1, such as 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1.

### IV. In Vivo Transfection

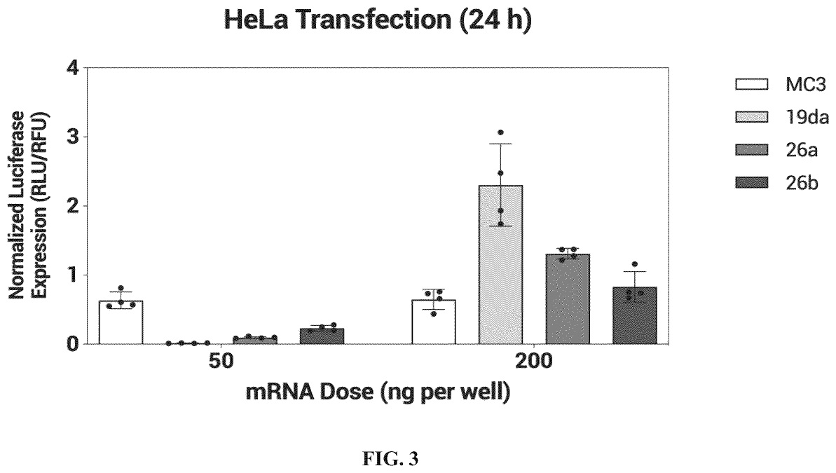

The lipids of the nanoparticle may facilitate transfection, for example, by interacting with endosomal membranes. Although all lipids may be involved in transfection, the ionizable lipid may be the main component responsible for gene delivery due to, e.g., the electrostatic attraction between the negatively charged endosomal membrane and positively charged ionizable lipid in the LNPs. Additionally, the nanoparticle composition may induce cytotoxicity. Transfection efficiency, cytotoxicity, and variability of these parameters as a function of administered dose are valuable metrics to evaluate the feasibility of in vivo studies. Selected transfection results are shown in FIGS. 3 to 5.

In some embodiments, the effectiveness of a particular nanoparticle may be measured by any suitable metric, such as, but not limited to, polypeptide or protein translation (indicated by polypeptide or protein expression). The various amount of nanoparticles and/or associated nucleic acid introduced may produce various levels of polypeptide or protein expression depending on the dose (the amount of the nanoparticle and/or associated nucleic acid introduced to the cells). Additionally, a nanoparticle composition and/or associated nucleic acid may induce cytotoxicity, or noticeable extent of cell injury and death upon exposure to the nanoparticle and/or associated nucleic acid. In some embodiments, a desirable cell viability is at least 50%.

### V. In Vivo Formulation Studies

To evaluate how effectively various nanoparticle compositions deliver therapeutic and/or prophylactic nucleic acids to target cells, different nanoparticle compositions were prepared and administered to rodents. In certain embodiments, mice receive a single dose of LNPs via intravenous, intramuscular, intradermal, or other administration routes. Dose sizes usually range from 0.05 mg/kg to 10 mg/kg or more, where 10 mg/kg describes a dose including 10 mg of a nucleic acid in a nanoparticle for each 1 kg of body mass of the mouse. Upon administration of nanoparticle compositions to mice, dose delivery profiles and dose responses were measured by bioluminescence imaging. For nanoparticle compositions including mRNA, time courses of protein expression can also be assessed. Higher levels of protein expression induced may indicate higher mRNA translation and/or enhanced mRNA delivery efficiency via a given nanoparticle composition. As the non-RNA components are not considered to produce protein expression themselves, a level of protein expression is likely indicative of a delivery efficiency of a nanoparticle composition including nucleic acids.

### VI. Methods of Using the Compounds and Compositions

Nanoparticles comprising one or more of the disclosed compounds may be used to deliver a desired nucleic acid to a subject, such as a human or animal subject. The amount of the nanoparticle administered to the subject can be determined by a person of ordinary skill in the art and may depend on the amount of the nucleic acid to be delivered and the ratio of nucleic acid to lipid, as described herein. Dose sizes may range from 0.05 mg/kg to 10 mg/kg or more, where 10 mg/kg describes a dose including 10 mg of a nucleic acid in a nanoparticle for each 1 kg of body mass of the subject.

Exemplary administration routes include any route suitable to administer the nanoparticle to subject, such as intravenous, intramuscular, intradermal, subcutaneous, intravitreous, subretinal, inhalation or a combination thereof. The nanoparticles are provided in physiological buffers, for example, phosphate buffered saline (PBS), and Hank's balanced salt solution (HBSS), at pH 7.0-7.6.

The compositions described herein may further comprise excipients, e.g., pharmaceutically acceptable excipients. Lipid nanoparticle formulations may also contain additional pharmaceutical excipients including, but not limited to, diluents, binders, and stabilizers of natural, semisynthetic, and/or synthetic origin. Some examples of these excipients include sugars, such as lactose, sucrose, trehalose, glucose, dextrin; naturally occurring polymers and starches, such as cellulose, chitosan, and derivatives; and synthetic polymers, such as polyethylene glycols, poloxamers, and polyamides.

In another aspect, the present disclosure provides a method of delivering a pharmaceutical agent to a subject in need thereof comprising administering to or implanting in the subject in need thereof an effective amount of:
a compound or pharmaceutical composition described herein; and
a pharmaceutical agent.

In another aspect, the present disclosure provides a method of treating a disease comprising administering to or implanting in a subject in need thereof an effective amount of:
a compound or pharmaceutical composition described herein; and
a therapeutic agent.

In another aspect, the present disclosure provides a method of preventing a disease comprising administering to or implanting in a subject in need thereof an effective amount of:
a compound or pharmaceutical composition described herein; and
a prophylactic agent.

In another aspect, the present disclosure provides a method of diagnosing a disease comprising administering to or implanting in a subject in need thereof an effective amount of:
a compound or pharmaceutical composition described herein; and
a diagnostic agent.

In certain embodiments, the pharmaceutical agent (e.g., the therapeutic agent, prophylactic agent, diagnostic agent) is an additional pharmaceutical agent described herein. In certain embodiments, the therapeutic agent is a pharmaceutical agent approved by the U.S. Food and Drug Administration or the European Medicines Agency for treating a disease in a subject. In certain embodiments, the prophylactic agent is a pharmaceutical agent approved by the U.S. Food and Drug Administration or the European Medicines Agency for preventing a disease in a subject. In certain embodiments, the diagnostic agent is a pharmaceutical agent approved by the U.S. Food and Drug Administration or the European Medicines Agency for diagnosing a disease in a subject.

In certain embodiments, the disease is an ocular disease. In certain embodiments, the disease is Aicardi syndrome, allergic eye disease, aphakia, autoimmune eye disease, Benign mucous membrane pemphigoid, blepharophimosis syndrome, blindness, buphthalmia, CHARGE syndrome, cataract, chalazion, choroid disease, Cogan syndrome, Cohen syndrome, conjunctiva disease, corneal disease, cycloplegia, dry eye syndrome, Duane syndrome, ectopia lentis, exophthalmos, eye hemorrhage, eye infection, eye injury, eye irritation, eye lesion, eye neoplasm, eye pain, eyelid disease, Fuchs heterochromic iridocyclitis, glaucoma, Harada syndrome, incontinentia pigmenti, iridopathy, Jalili syndrome, keratoconus, lacrimal gland disease, Leber congenital amaurosis, Mainzer-Saldino syndrome, Miller Fisher syndrome, miosis, mydriasis, night blindness, nystagmus, ocular albinism, ocular edema, ocular fibrosis, ocular hypertension, ocular hypotension, ocular ischemia, ocular neovascularization, ocular photophobia, ocular rosacea, ocular synechia, oculoauriculovertebral dysplasia, oculopharyngeal muscular dystrophy, ophthalmia, ophthalmitis, ophthalmoplegia, opsoclonus, optic atrophy, osteoporosis-pseudoglioma syndrome, paraneoplastic ocular syndrome, Peters anomaly, phlyctenule, posterior capsule opacification, pseudoinflammatory fundus dystrophy, pseudophakia, pupil disease, retinal disease, Rieger syndrome, Rothmund-Thomson syndrome, Senior-Loken syndrome, Sjogren syndrome, Sorsby fundus dystrophy, tonic pupil, or uveal disease. In certain embodiments, the disease is inherited retinal disease. In certain embodiments, the disease is choroideremia, polypoidal choroidal vasculopathy, or retinal photoreceptor degeneration. In certain embodiments, the disease is retinal degeneration. In certain embodiments, the disease is inherited retinal degeneration. In certain embodiments, the disease is maculopathy. In certain embodiments, the disease is macular degeneration (e.g., age-related macular degeneration). In certain embodiments, the disease is macular dystrophy, macular edema, or macular hole.

In certain embodiments, the disease is cystic fibrosis.

In certain embodiments, the disease is cancer, benign neoplasm, pathologic angiogenesis, inflammatory disease, autoinflammatory disease, autoimmune disease, metabolic disease, neurological disease, painful condition, or psychiatric disease. In certain embodiments, the disease is cancer.

In certain embodiments, the subject is a human. In certain embodiments, the subject is a human aged 18 years and older. In some embodiments, the subject is a human aged<2 years. In some embodiments, the subject is a human aged 2-6 years, inclusive. In some embodiments, the subject is a human aged 6-18 years, inclusive.

### VII. Methods of Making the Compounds

The compounds provided herein may be prepared by methods known in the art.

In another aspect, the present disclosure provides a method of making the compounds of Formula II-a also is disclosed herein, the method comprises:
when R⁵ is H: reacting a compound of the formula:

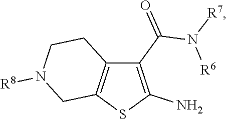

with a compound of the formula: R⁴-(leaving group) or R⁴—OH under suitable conditions to provide the compound; or
when R⁵ is aliphatic or —C(O)aliphatic: reacting a compound of the formula:

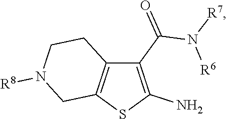

with a compound of the formula: R⁴-(leaving group) or R⁴—OH, and a compound of the formula: R⁵-(leaving group) or R⁵—OH, in the same step or separate steps, under suitable conditions to provide the compound.

In certain embodiments, the method further comprising reacting a compound of the formula:

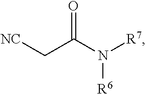

with a compound of the formula:

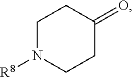

in the presence of S₈ under suitable conditions to provide the compound of the formula:

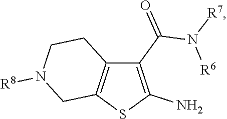

In certain embodiments, R⁴ is —C(O)aliphatic. In certain embodiments, R⁵ is H. In certain embodiments, R⁶ is H. In certain embodiments, R⁷ is aliphatic. In certain embodiments, R⁸ is —C(O)aliphatic.

In certain embodiments, the suitable condition comprises a base. In certain embodiments, the base is an organic base. In certain embodiments, the base is a mono-, di-, or tri-(unsubstituted C₁₋₆ alkyl) amine. In certain embodiments, the base is a tri-(unsubstituted C₁₋₆ alkyl) amine. In certain embodiments, the base is trimethylamine, triethylamine, or N,N-diisopropylethylamine, or a mixture thereof. In certain embodiments, the base is a cyclic non-aromatic amine. In certain embodiments, the base is an aromatic amine (e.g., pyridine). In certain embodiments, the base is an inorganic base. In certain embodiments, the base is Li₂CO₃, Na₂CO₃, or K₂CO₃, or a mixture thereof. In certain embodiments, the base is LiHCO₃, NaHCO₃, or KHCO₃, or a mixture thereof. In certain embodiments, the base is ammonia, ammonium carbonate, or ammonium hydroxide.

In certain embodiments, the suitable condition comprises an amide coupling agent. In certain embodiments, the amide coupling agent is 1-[bis(dimethylamino)methylene]-1H-1,2,3-triazolo[4,5-b]pyridinium 3-oxide hexafluorophosphate, benzotriazole-1-yl-oxy-tris-(dimethylamino)-phosphonium hexafluorophosphate, 1-[(1-(cyano-2-ethoxy-2-oxoethylideneaminooxy) dimethylaminomorpholino)] uronium hexafluorophosphate, 2-(1H-benzotriazole-1-yl)-1,1,3,3-tetramethyluronium hexafluorophosphate, 0-(1H-6-chlorobenzotriazole-1-yl)-1,1,3,3-tetramethyluronium hexafluorophosphate, (7-azabenzotriazol-1-yloxy)trispyrrolidinophosphonium hexafluorophosphate, benzotriazole-1-yl-oxy-tris-pyrrolidino-phosphonium hexafluorophosphate, 6-chloro-benzotriazole-1-yloxy-tris-pyrrolidinophosphonium hexafluorophosphate, 1-cyano-2-ethoxy-2-oxoethylideneaminooxy-tris-pyrrolidino-phosphonium hexafluorophosphate, or O-[(ethoxycarbonyl)cyanomethylenamino]-N,N,N′,N′-tetramethyluronium tetrafluoroborate.

In certain embodiments, the suitable condition comprises substantially no solvents. In certain embodiments, the suitable condition comprises a solvent. In certain embodiments, the solvent is substantially one single solvent. In certain embodiments, the solvent is a mixture of two or more (e.g., three) solvents. In certain embodiments, the solvent is an organic solvent. In certain embodiments, the solvent is a non-aromatic organic solvent. In certain embodiments, the solvent is an aprotic solvent. In certain embodiments, the solvent is an ether solvent (e.g., diethyl ether, methyl t-butyl ether, tetrahydrofuran, 2-methyltetrahydrofuran, 1,4-dioxane, or a mixture thereof). In certain embodiments, the solvent is a ketone solvent (e.g., acetone, methyl ethyl ketone, methyl isopropyl ketone or a mixture thereof). In certain embodiments, the solvent is an alkane solvent (e.g., a pentane, a hexane, a heptane, a petroleum ether, or a mixture thereof). In certain embodiments, the solvent is a haloalkane solvent (e.g., CCl₄, chloroform, dichloromethane, or a mixture thereof). In certain embodiments, the solvent is an amide or sulfoxide solvent (e.g., N,N-dimethylformamide, N,N-dimethylacetamide, dimethyl sulfoxide, or a mixture thereof. In certain embodiments, the solvent is an aromatic organic solvent (e.g., benzene, toluene, o-xylene, m-xylene, or p-xylene, or a mixture thereof). In certain embodiments, the solvent is acetonitrile or ethyl acetate. In certain embodiments, the solvent is a protic solvent. In certain embodiments, the solvent is an alcohol solvent (e.g., methanol, ethanol, n-propanol, isopropanol, t-butanol, or a mixture thereof). In certain embodiments, the solvent is an inorganic solvent (e.g., water). In certain embodiments, the solvent is a mixture of an ether solvent (e.g., tetrahydrofuran) and a haloalkane solvent (e.g., dichloromethane). In certain embodiments, the volume ratio of a mixture of two solvents is between 1:10 and 1:5, between 1:5 and 1:3, between 1:3 and 1:2, between 1:2 and 1:1, between 1:1 and 2:1, between 2:1 and 3:1, between 3:1 and 5:1, or between 5:1 and 10:1, inclusive.

In certain embodiments, the boiling point of the solvent at about 1 atm is between 30 and 50, between 50 and 70, between 70 and 100, between 100 and 130, between 130 and 160, or between 160 and 200° C., inclusive.

In certain embodiments, the suitable condition is substantially free of dioxygen. In certain embodiments, the suitable condition is substantially free of water.

In certain embodiments, the suitable condition comprises a reaction temperature. In certain embodiments, the reaction temperature is between 20 and 30, between 30 and 40, between 40 and 50, between 50 and 60, between 60 and 70, or between 70 and 80° C., e.g., between 40 and 60° C.

In certain embodiments, the suitable condition comprises a reaction time duration. In certain embodiments, the reaction time duration is between 1 and 3 hours, between 3 and 8 hours, between and 24 hours, between 1 and 2 days, between 2 and 4 days, or between 4 and 7 days, e.g., between 6 and 24 hours.

In certain embodiments, the suitable condition comprises a reaction pressure. In certain embodiments, the reaction pressure is between 0.5 and 1.1 atm (e.g., between 0.8 and 1.1 atm).

The methods of making the compounds provided herein may be advantageous over the methods known in the art at least in part because the former is a more modular, higher yielding, easier to perform or purify, cheaper, faster, more environment friendly, and/or involves fewer steps, less harsh reaction conditions, or fewer by-products.

### VIII. Kits

Also encompassed by the disclosure are kits (e.g., pharmaceutical packs) comprising a compound or composition provided herein; and instructions for using the compound or composition provided herein. In some embodiments, the kit comprises a composition or compound described herein and a container (e.g., a vial, ampule, bottle, syringe, and/or dispenser package, or other suitable container). In some embodiments, provided kits optionally further include a second container comprising an excipient for dilution or suspension of a composition or compound described herein. In some embodiments, the composition or compound described herein provided in the first container and the second container are combined to form one unit dosage form.

In certain embodiments, the kit includes a first container comprising a compound or composition provided herein. In certain embodiments, the kit includes a second container comprising an agent (e.g., pharmaceutical agent).

In certain embodiments, the kits are useful for delivering the agent to a subject in need thereof, cell, tissue, or biological sample. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit provided herein further includes instructions for using compound or composition. A kit described herein may also include information as required by a regulatory agency such as the U.S. Food and Drug Administration (FDA). In certain embodiments, the information included in the kits is prescribing information. In certain embodiments, the kits and instructions provide for delivering the agent to a subject in need thereof, cell, tissue, or biological sample. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof.

### IX. Examples

### Example 1

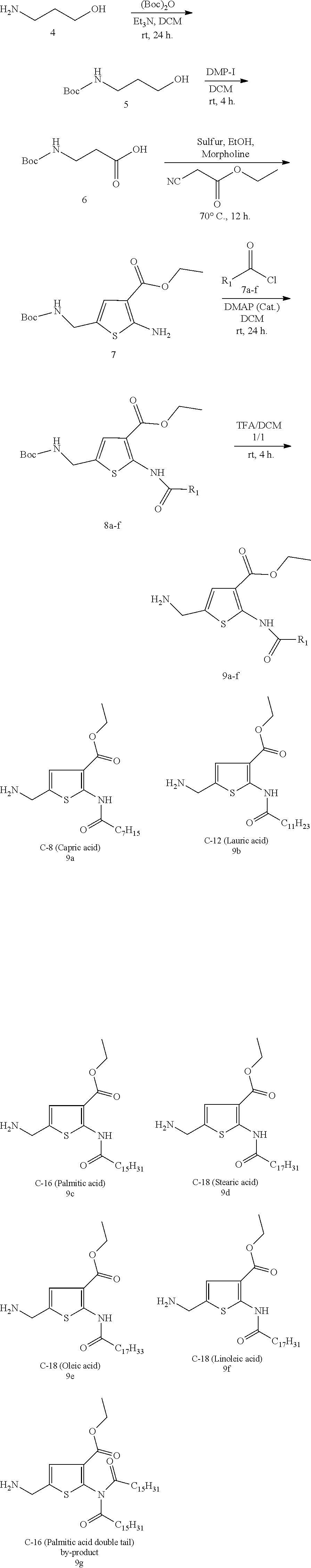

Synthesis of 5, tert-butyl (3-hydroxypropyl)carbamate: An overnight dried round bottom flask (RBF) was charged with 1-aminopropanol (0.76 mL, 10 mmol) and anhydrous dichloromethane (10 mL). To this was added anhydrous triethylamine (1.67 mL, 12 mmol) under a nitrogen atmosphere and the reaction mixture was cooled to 0° C. using an ice bath. To this di-tert-butyl dicarbonate (2.18 gm, 10 mmol) was added in portions. The reaction mixture was allowed to warm to room temperature and stirred for an additional 24 hours under a nitrogen atmosphere. Subsequently, the reaction mixture was diluted with dichloromethane (50 mL), washed with 0.1 M hydrochloric acid (10 mL×2), water (10 mL), and brine (10 mL), dried over MgSO₄, and concentrated under vacuum to yield a colorless oil (900 mg, yield 51%). The NMR matched the literature.

Synthesis of 6, tert-butyl (3-oxopropyl)carbamate: An overnight dried round bottom flask (RBF) was charged with compound 5 (0.82 gm, 4.68 mmol) and anhydrous dichloromethane (10 mL). The reaction mixture was cooled to 0° C. under a nitrogen atmosphere. To this, Dess-Martin periodinane (2.38 gm, 5.6 mmol) was added in portions over 10 minutes. The reaction mixture was stirred at 0° C. for 15 minutes, then warmed to room temperature and stirred for an additional 4 hours. Following this, the reaction mixture was diluted with diethyl ether (100 mL), 10% sodium thiosulphate solution (30 mL), and saturated sodium bicarbonate solution (30 ml). The resulting suspension was stirred vigorously until the precipitate was fully dissolved. The organic and aqueous layers were separated, and the aqueous layer was extracted with diethyl ether (2×60 mL). The organic layers were combined and washed with 10% sodium thiosulphate solution (2×30 mL), saturated sodium bicarbonate solution (2×30 mL), and brine (30 mL), dried over MgSO4, and concentrated in vacuo to produce a slightly yellow oil (580 mg, yield 70%) which was used without further purification.

Synthesis of 7, ethyl 2-amino-5-(((tert-butoxycarbonyl)amino)methyl)thiophene-3-carboxylate: To an over-night dried RBF was added 6 (580 mg, 3.35 mmol), sulfur (107.2 mg, 3.35 mmol), and ethyl cyanoacetate (0.35 mL, 3.35 mmol) in anhydrous ethanol (35 mL). The reaction mixture was stirred at room temperature for 30 minutes. Following this, morpholine (0.35 mL, 4 mmol) was added, and the reaction mixture was allowed to stir for an additional 15 minutes at room temperature. After this, the reaction mixture was heated at 70° C. for 12 hours. The reaction mixture was cooled, and the solvent was removed under reduced pressure. The crude was purified using SiO₂ chromatography to produce ethyl 2-amino-5-(((tert-butoxycarbonyl)amino)methyl)thiophene-3-carboxylate (7) as a yellow semi-solid (571 mg, yield 59%).

General protocol for the acylation reaction (Procedure A): A dry 100 mL round-bottomed flask was charged with a suitable carboxylic acid (2 mmol) and 10 mL of DCM. The reaction mixture was cooled with an ice bath to 0° C. A 100 μL volume of anhydrous DMF was added, followed by dropwise addition of neat oxalyl chloride (2.4 mmol) over 5 minutes. The reaction was stirred at room temperature for 4 hours. The solvent was removed under reduced pressure. Excess oxalyl chloride was azeotropically removed with 2×5 mL portions of DCM under reduced pressure. The crude acyl chloride was used without further purification. The crude acyl chloride was dissolved into 10 mL of DCM, and the solution was cooled with an ice bath to 0° C. To this, DMAP (5 mg, catalytic) and a solution of 7 (300 mg, 1 mmol) in 3 mL of DCM were added under nitrogen atmosphere. The reaction was stirred at 0° C. for 15 minutes, after which triethylamine (1 mL, excess) was added in portions. The ice bath was removed after 10 minutes, and the reaction was permitted to warm to room temperature. The reaction was then stirred at room temperature for 24 hours and the completion of the reaction was determined by TLC. The solvent was removed under reduced pressure. The crude product was purified using SiO₂ chromatography using hexane/ethyl acetate (EA) as a solvent.

### Synthesis of 8a-g:

8a, ethyl 5-(((tert-butoxycarbonyl)amino)methyl)-2-octanamidothiophene-3-carboxylate was synthesized using the general procedure A from octanoyl chloride (7a, 324 mg, 2 mmol) [synthesized from octanoic acid (288 mg, 2.0 mmol), 100 μL DMF, oxalyl chloride (205 μL, 2.4 mmol), and DCM (10 mL)], analog 7 (300 mg, 1 mmol), DMAP (5 mg, catalytic), triethylamine (1 mL, excess), and DCM (13 mL). The crude product was isolated on SiO₂ using 5:1 hexane/EA to give 188 mg (44%) of a yellow oil.

8b, ethyl 5-(((tert-butoxycarbonyl)amino)methyl)-2-dodecanamidothiophene-3-carboxylate was synthesized using the general procedure A from dodecanoyl chloride (7b, 436 mg, 2 mmol) [synthesized from dodecanoic acid (400 mg, 2.0 mmol), 100 μL DMF, oxalyl chloride (205 μL, 2.4 mmol), and DCM (10 mL)], analog 7 (300 mg, 1 mmol), DMAP (5 mg, catalytic), triethylamine (1 mL, excess), and DCM (13 mL). The crude product was isolated on SiO₂ using 5:1 hexane/EA to give 146 mg (30%) of a yellow powder.

8c, ethyl 5-(((tert-butoxycarbonyl)amino)methyl)-2-palmitamidothiophene-3-carboxylate was synthesized using the general procedure A from palmitoyl chloride (7c, 548 mg, 2 mmol) [synthesized from palmitic acid (512 mg, 2.0 mmol), 100 μL DMF, oxalyl chloride (205 μL, 2.4 mmol), and DCM (10 mL)], analog 7 (300 mg, 1 mmol), DMAP (5 mg, catalytic), triethylamine (1 mL, excess), and DCM (13 mL). The crude product was isolated on SiO₂ using 5:1 hexane/EA to give 192 mg (35%) of a yellow powder.

8d, ethyl 5-(((tert-butoxycarbonyl)amino)methyl)-2-stearamidothiophene-3-carboxylate was synthesized using the general procedure A from stearoyl chloride (7d, 606 mg, 2 mmol) [synthesized from stearic acid (569 mg, 2.0 mmol), 100 μL DMF, oxalyl chloride (205 μL, 2.4 mmol), and DCM (10 mL)], analog 7 (300 mg, 1 mmol), DMAP (5 mg, catalytic), triethylamine (1 mL, excess), and DCM (13 mL). The crude product was isolated on SiO₂ using 5:1 hexane/EA to give 155 mg (27%) of a bright-yellow powder.

8e, ethyl 5-(((tert-butoxycarbonyl)amino)methyl)-2-oleamidothiophene-3-carboxylate was synthesized using the general procedure A from oleoyl chloride (7e, 602 mg, 2 mmol) [synthesized from oleic acid (565 mg, 2.0 mmol), 100 μL DMF, oxalyl chloride (205 μL, 2.4 mmol), and DCM (10 mL)], analog 7 (300 mg, 1 mmol), DMAP (5 mg, catalytic), triethylamine (1 mL, excess), and DCM (13 mL). The crude product was isolated on SiO₂ using 5:1 hexane/EA to give 169 mg (30%) of a yellow oil.

8f, ethyl 5-(((tert-butoxycarbonyl)amino)methyl)-2-((9Z,12Z)-octadeca-9,12-dienamido)thiophene-3-carboxylate was synthesized using the general procedure A from linoleyl chloride (7f, 598 mg, 2 mmol) [synthesized from linoleic acid (561 mg, 2.0 mmol), 100 μL DMF, oxalyl chloride (205 μL, 2.4 mmol), and DCM (10 mL)], analog 7 (300 mg, 1 mmol), DMAP (5 mg, catalytic), triethylamine (1 mL, excess), and DCM (13 mL). The crude product was isolated on SiO₂ using 5:1 hexane/EA to give 121 mg (21%) of a yellow oil.

8g, ethyl 5-(((tert-butoxycarbonyl)amino)methyl)-2-(N-palmitoylpalmitamido) thiophene-3-carboxylate was isolated as a by-product during the synthesis of 8c as a yellow oil, 35 mg (4%).

General protocol for the Boc-deprotection (Procedure B): In an overnight dried round bottom flask was added Boc-protected amine and TFA:DCM (1:1, 6 mL) in a nitrogen atmosphere. The reaction was stirred at room temperature for 4 hours, after which the solvent was removed under reduced pressure to produce TFA salt of the desired amine.

Synthesis of 9a-9g:

9a, ethyl 5-(aminomethyl)-2-octanamidothiophene-3-carboxylate TFA salt was synthesized using the general procedure B from 8a (50 mg, 0.11 mmol) and TFA:DCM (1:1, 6 mL). Removal of the solvent produced 46 mg (90%) of a light-brown powder.

9b, ethyl 5-(aminomethyl)-2-dodecanamidothiophene-3-carboxylate TFA salt was synthesized using the general procedure B from 8b (50 mg, 0.10 mmol) and TFA:DCM (1:1, 6 mL). Removal of the solvent produced 47 mg (91%) of a light-yellow powder.

9c, ethyl 5-(aminomethyl)-2-palmitamidothiophene-3-carboxylate TFA salt was synthesized using the general procedure B from 8c (50 mg, 0.09 mmol) and TFA:DCM (1:1, 6 mL). Removal of the solvent produced 44 mg (86%) of a white powder.

9d, ethyl 5-(aminomethyl)-2-stearamidothiophene-3-carboxylate TFA salt was synthesized using the general procedure B from 8d (50 mg, 0.09 mmol) and TFA:DCM (1:1, 6 mL). Removal of the solvent produced 49 mg (95%) of a yellow powder.

9e, ethyl 5-(aminomethyl)-2-oleamidothiophene-3-carboxylate TFA salt was synthesized using the general procedure B from 8e (50 mg, 0.09 mmol) and TFA:DCM (1:1, 6 mL). Removal of the solvent produced 50 mg (98%) of a yellow powder.

9f, ethyl 5-(aminomethyl)-2-((9Z,12Z)-octadeca-9,12-dienamido)thiophene-3-carboxylate TFA salt was synthesized using the general procedure B from 8f (50 mg, 0.09 mmol) and TFA:DCM (1:1, 6 mL). Removal of the solvent produced 50 mg (98%) of a yellow powder.

9g, ethyl 5-(aminomethyl)-2-(N-palmitoylpalmitamido)thiophene-3-carboxylate TFA salt was synthesized using the general procedure B from 8g (50 mg, 0.06 mmol) and TFA:DCM (1:1, 6 mL). Removal of the solvent produced 48 mg (94%) of a yellow powder.

### Example 2

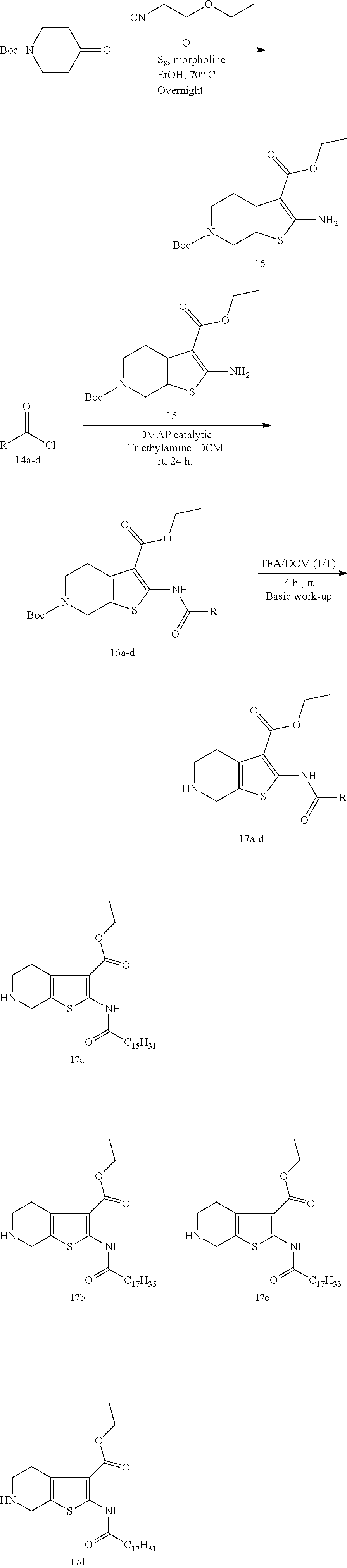

Synthesis of 15, 6-(tert-butyl)-3-ethyl 2-amino-4,7-dihydrothieno[2,3-c] pyridine-3,6(5H)-dicarboxylate: A 250 mL one-neck round bottom flask was charged with ethyl cyanoacetate (3.2 mL, 24 mmol), tert-butyl 4-oxopiperidine-1-carboxylate (3.7 g, 20 mmol), sulfur (768 mg, 24 mmol), morpholine (5 mL, excess), and ethanol (50 mL). The mixture was stirred at 70 8° C. for 12 hours. The reaction mixture was cooled, filtered, and washed with cold EtOH followed by water. The solid collected was dried to yield 5.35 g (82%) of a light yellow crystalline solid. ¹H NMR (400 MHz, CDCl3): δ 6.06 (s, 2H), 4.36 (s, 2H), 4.29 (q, J=7.2 Hz, 2H), 3.63 (t, 2H), 2.82 (s, 2H), 1.49 (s, 9H), 1.35 (t, J=7.2 Hz, 3H), matching the literature.

### Synthesis of 16a-d:

16a, 6-(tert-butyl) 3-ethyl 2-palmitamido-4,7-dihydrothieno[2,3-c]pyridine-3,6(5H)-dicarboxylate was synthesized using the general procedure A from palmitoyl chloride (14a, 548 mg, 2 mmol) [synthesized from palmitic acid (512 mg, 2.0 mmol), 100 μL DMF, oxalyl chloride (205 μL, 2.4 mmol), and DCM (10 mL)], analog 15 (326 mg, 1 mmol), DMAP (5 mg, catalytic), triethylamine (1 mL, excess), and DCM (13 mL). The crude product was isolated on SiO₂ using 5:1 hexane/EA to give 212 mg (37%) of a light-yellow oil.

16b, 6-(tert-butyl) 3-ethyl 2-stearamido-4,7-dihydrothieno[2,3-c]pyridine-3,6(5H)-dicarboxylate was synthesized using the general procedure A from stearoyl chloride (14b, 606 mg, mmol) [synthesized from stearic acid (569 mg, 2.0 mmol), 100 μL DMF, oxalyl chloride (205 μL, 2.4 mmol), and DCM (10 mL)], analog 15 (326 mg, 1 mmol), DMAP (5 mg, catalytic), triethylamine (1 mL, excess), and DCM (13 mL). The crude product was isolated on SiO₂ using 5:1 hexane/EA to give 208 mg (35%) of a yellow film.

16c, 6-(tert-butyl) 3-ethyl 2-oleamido-4,7-dihydrothieno[2,3-c]pyridine-3,6(5H)-dicarboxylate was synthesized using the general procedure A from oleoyl chloride (14c, 602 mg, 2 mmol) [synthesized from oleic acid (565 mg, 2.0 mmol), 100 μL DMF, oxalyl chloride (205 μL, 2.4 mmol), and DCM (10 mL)], analog 15 (326 mg, 1 mmol), DMAP (5 mg, catalytic), triethylamine (1 mL, excess), and DCM (13 mL). The crude product was isolated on SiO₂ using 5:1 hexane/EA to give 200 mg (34%) of a yellow oil.

16d, 6-(tert-butyl) 3-ethyl 2-((9Z,12Z)-octadeca-9,12-dienamido)-4,7-dihydrothieno[2,3-c]pyridine-3,6(5H)-dicarboxylate was synthesized using the general procedure A from linoleyl chloride (14d, 598 mg, 2 mmol) [synthesized from linoleic acid (561 mg, 2.0 mmol), 100 μL DMF, oxalyl chloride (205 μL, 2.4 mmol), and DCM (10 mL)], analog 15 (326 mg, 1 mmol), DMAP (5 mg, catalytic), triethylamine (1 mL, excess), and DCM (13 mL). The crude product was isolated on SiO₂ using 5:1 hexane/EA to give 194 mg (33%) of a yellow oil.

### Synthesis of 17a-d:

17a, ethyl 2-palmitamido-4,5,6,7-tetrahydrothieno[2,3-c]pyridine-3-carboxylate was synthesized using the general procedure B from 16a (100 mg, 0.17 mmol) and TFA:DCM (1:1, 6 mL). The resulting solution was diluted with DCM (20 mL), washed with 0.5 N NaOH (pH ˜12) and brine (10 mL), dried over MgSO4, and concentrated under vacuum to yield 62 mg (75%) of a white powder.

17b, ethyl 2-stearamido-4,5,6,7-tetrahydrothieno[2,3-c]pyridine-3-carboxylate was synthesized using the general procedure B from 16b (100 mg, 0.17 mmol) and TFA:DCM (1:1, 6 mL). The resulting solution was diluted with DCM (20 mL), washed with 0.5 N NaOH (pH ˜12), and brine (10 mL), dried over MgSO₄, and concentrated under vacuum to yield 56 mg (67%) of a yellow powder.

17c, ethyl 2-oleamido-4,5,6,7-tetrahydrothieno[2,3-c]pyridine-3-carboxylate was synthesized using the general procedure B from 16c (100 mg, 0.17 mmol) and TFA:DCM (1:1, 6 mL). The resulting solution was diluted with DCM (20 mL), washed with 0.5 N NaOH (pH ˜12) and brine (10 mL), dried over MgSO₄, and concentrated under vacuum to yield 65 mg (78%) of a yellow film.

17d, ethyl 2-((9Z,12Z)-octadeca-9,12-dienamido)-4,5,6,7-tetrahydrothieno[2,3-c]pyridine-3-carboxylate was synthesized using the general procedure B from 16d (100 mg, 0.17 mmol) and TFA:DCM (1:1, 6 mL). The resulting solution was diluted with DCM (20 mL), washed with 0.5 N NaOH (pH ˜12), and brine (10 mL), dried over MgSO₄, and concentrated under vacuum to yield 48 mg (58%) of a yellow oil.

### Example 3

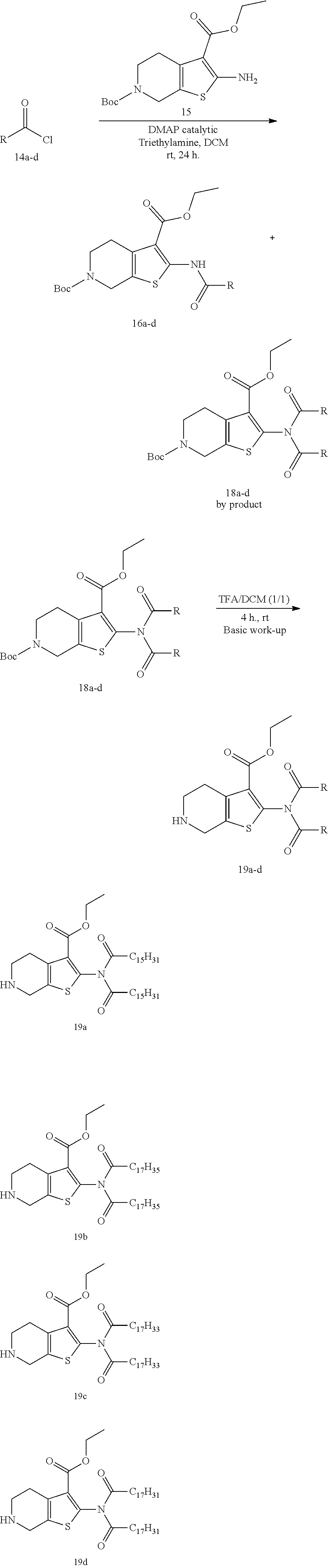

### Synthesis of 18a-d:

18a, 6-(tert-butyl) 3-ethyl 2-(N-palmitoylpalmitamido)-4,7-dihydrothieno[2,3-c]pyridine-3,6(5H)-dicarboxylate was isolated as a by-product during the synthesis of 16a as a yellow film, 102 mg (12%).

18b, 6-(tert-butyl) 3-ethyl 2-(N-stearoylstearamido)-4,7-dihydrothieno[2,3-c]pyridine-3,6(5H)-dicarboxylate was isolated as a by-product during the synthesis of 16b as a yellow powder, 68 mg (8%).

18c, 6-(tert-butyl) 3-ethyl 2-(N-oleoyloleamido)-4,7-dihydrothieno[2,3-c]pyridine-3,6(5H)-dicarboxylate was isolated as a by-product during the synthesis of 16c as a yellow oil, 96 mg (11%).

18d, 6-(tert-butyl) 3-ethyl 2-((9Z,12Z)—N-((9Z,12Z)-octadeca-9,12-dienoyl)octadeca-9,12-dienamido)-4,7-dihydrothieno[2,3-c]pyridine-3,6(5H)-dicarboxylate was isolated as a by-product during the synthesis of 16d as a yellow oil, 90 mg (10%).

### Synthesis of 19a-d:

19a, ethyl 2-(N-palmitoylpalmitamido)-4,5,6,7-tetrahydrothieno[2,3-c]pyridine-3-carboxylate was synthesized using the general procedure B from 18a (100 mg, 0.12 mmol) and TFA:DCM (1:1, 6 mL). The resulting solution was diluted with DCM (20 mL), washed with 0.5 N NaOH (pH ˜12) and brine (10 mL), dried over MgSO₄, and concentrated under vacuum to yield 68 mg (78%) of a yellow powder.

19b, ethyl 2-(N-stearoylstearamido)-4,5,6,7-tetrahydrothieno[2,3-c]pyridine-3-carboxylate was synthesized using the general procedure B from 18b (100 mg, 0.11 mmol) and TFA:DCM (1:1, 6 mL). The resulting solution was diluted with DCM (20 mL), washed with 0.5 N NaOH (pH ˜12), and brine (10 mL), dried over MgSO₄, and concentrated under vacuum to yield 62 mg (70%) of a yellow powder.

19c, ethyl 2-(N-oleoyloleamido)-4,5,6,7-tetrahydrothieno[2,3-c]pyridine-3-carboxylate was synthesized using the general procedure B from 18c (100 mg, 0.11 mmol) and TFA:DCM (1:1, 6 mL). The resulting solution was diluted with DCM (20 mL), washed with 0.5 N NaOH (pH ˜12) and brine (10 mL), dried over MgSO₄, and concentrated under vacuum to yield 66 mg (75%) of a yellow powder.

19d, ethyl 2-((9Z,12Z)—N-((9Z,12Z)-octadeca-9,12-dienoyl)octadeca-9,12-dienamido)-4,5,6,7-tetrahydrothieno[2,3-c]pyridine-3-carboxylate was synthesized using the general procedure B from 18d (100 mg, 0.11 mmol) and TFA:DCM (1:1, 6 mL). The resulting solution was diluted with DCM (20 mL), washed with 0.5 N NaOH (pH ˜12), and brine (10 mL), dried over MgSO₄, and concentrated under vacuum to yield 70 mg (79%) of a yellow oil.

### Example 4

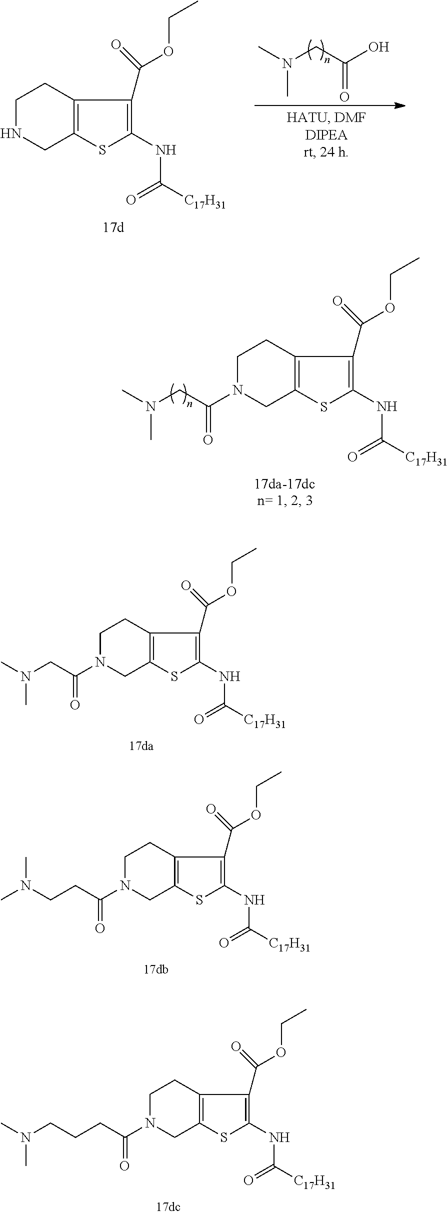

General protocol for the amide formation using HATU coupling (Procedure C): A solution of the amine (0.2 mmol), acid (0.6 mmol), DIPEA (105 μl, 0.6 mmol) in DMF (under argon) was stirred for 15 minutes at room temperature in a 25 mL RBF (overnight dried) under nitrogen atmosphere. HATU (228 mg, 0.6 mmol) was then added, and the reaction was allowed to stir for 24 hours (reaction progress monitored by TLC). The reaction mixture was diluted with water (30 mL) and extracted with DCM (15 mL×5). The organic layers were combined, washed with brine (30 mL), and dried over MgSO₄. The solvent was removed, and the crude was then purified by SiO₂ flash chromatography (Biotage® Isolera™) to obtain the desired product. For basic amines, a solvent system comprising of 1-9% methanol/DCM (1% of 7 N NH₃ additive) was used. For neutral analogs, a solvent system comprising of hexanes/ethyl acetate (6-50%) was used.

Synthesis of 17da-17dc:

17da, ethyl 6-(dimethylglycyl)-2-((9Z,12Z)-octadeca-9,12-dienamido)-4,5,6,7-tetrahydrothieno[2,3-c]pyridine-3-carboxylate: Using the general procedure C, with 17d (98 mg, 0.2 mmol), N,N-Dimethylglycine (62 mg, 0.6 mmol), DIPEA (105 μl, 0.6 mmol), HATU (228 mg, 0.6 mmol), and DMF (4 mL), 17da was prepared as a yellow solid, 28 mg (24%).

17db, ethyl 6-(3-(dimethylamino)propanoyl)-2-((9Z,12Z)-octadeca-9,12-dienamido)-4,5,6,7-tetrahydrothieno[2,3-c]pyridine-3-carboxylate: Using the general procedure C, with 17d (98 mg, 0.2 mmol), 3-(Dimethylamino)propionic acid hydrochloride (92 mg, 0.6 mmol), DIPEA (105 μl, 0.6 mmol), HATU (228 mg, 0.6 mmol), and DMF (4 mL), 17db was prepared as a yellow oil, 32 mg (27%).

17dc, ethyl 6-(4-(dimethylamino)butanoyl)-2-((9Z,12Z)-octadeca-9,12-dienamido)-4,5,6,7-tetrahydrothieno[2,3-c]pyridine-3-carboxylate: Using the general procedure C, with 17d (98 mg, 0.2 mmol), 4-(dimethylamino)butanoic acid hydrochloride (100 mg, 0.6 mmol), DIPEA (105 μl, 0.6 mmol), HATU (228 mg, 0.6 mmol), and DMF (4 mL), 17dc was prepared as a yellow oil, 41 mg (34%).

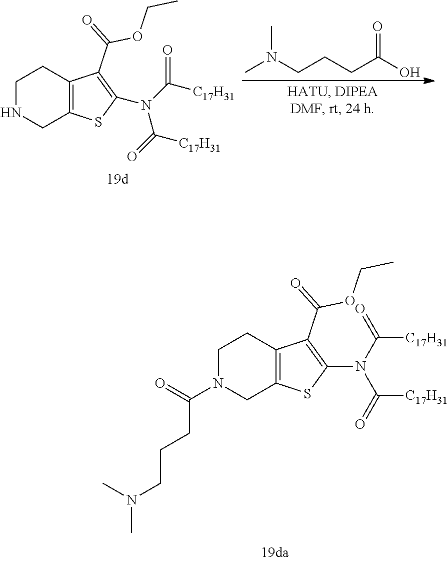

Synthesis of 19da, ethyl 6-(4-(dimethylamino)butanoyl)-2-((9Z,12Z)—N-((9Z,12Z)-octadeca-9,12-dienoyl)octadeca-9,12-dienamido)-4,5,6,7-tetrahydrothieno[2,3-c]pyridine-3-carboxylate: Using the general procedure C, with 19d (150 mg, 0.2 mmol), 4-(dimethylamino)butanoic acid hydrochloride (100 mg, 0.6 mmol), DIPEA (105 μl, 0.6 mmol), HATU (228 mg, 0.6 mmol), and DMF (4 mL), 19da was prepared as a yellow oil, 74 mg (43%).

### Example 5

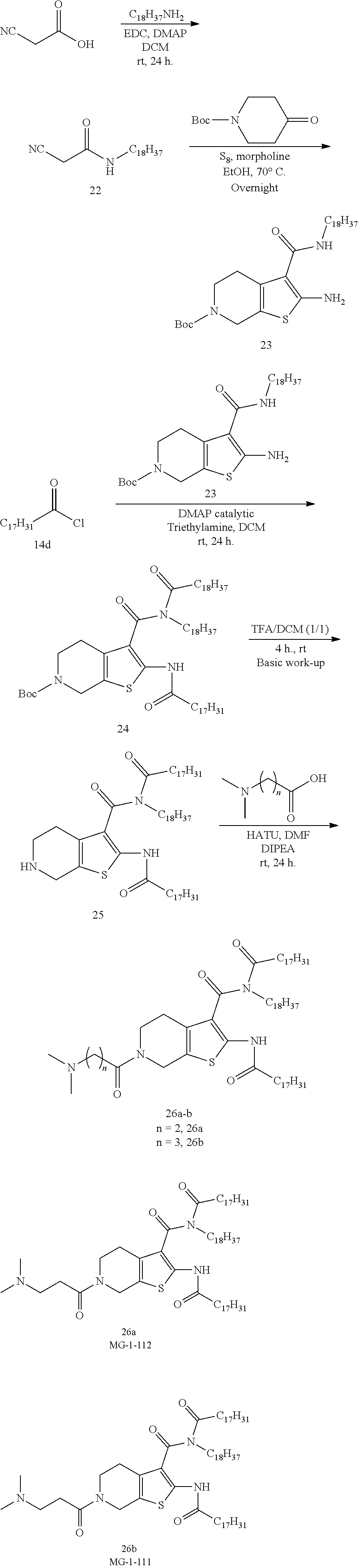

General protocol for EDC coupling (Procedure D): An oven-dried round-bottomed flask was charged with a suitable acid (1 eq.), amine (1.2 eq.), DMAP (catalytic), and DCM at room temperature under nitrogen atmosphere. The reaction was allowed to stir for 15 minutes, then EDC (N-(3-Dimethylaminopropyl)-N′-ethylcarbodiimide hydrochloride, 1.2 eq.) was added. The reaction mixture was stirred for an additional 24 hours at room temperature. After this, the crude was dissolved in 50 mL of DCM and washed with 3×20 mL of 0.5 N HCl, 2×20 mL of water, followed by brine wash. The crude was dried over Sodium sulfate and purified using SiO₂ flash chromatography (Biotage® Isolera™) with 10-50% of hexane/ethyl acetate to give rise to the final compound.

Synthesis of 22, 2-cyano-N-octadecylacetamide: Using the general procedure D, with cyanoacetic acid (2.98 gm, 35 mmol), stearyl amine (11.1 g, 35 mmol), DMAP (280 mg, 2.3 mmol), EDC (8 gm, 42 mmol), and DCM (100 mL), 22 was prepared as a yellow crystalline powder, 4.8 gm (41%).

Synthesis of 23, tert-butyl 2-amino-3-(octadecylcarbamoyl)-4,7-dihydrothieno[2,3-c]pyridine-6(5H)-carboxylate: A 250 mL one-neck round bottom flask was charged with 22 (2.6 gm, 7.7 mmol), tert-butyl 4-oxopiperidine-1-carboxylate (1.4 g, 7 mmol), sulfur (246 mg, 7.7 mmol), morpholine (1.5 mL, excess), and ethanol (20 mL). The mixture was stirred at 70° C. for 12 hours. The reaction mixture was cooled, and the solvent was evaporated under reduced pressure to give rise to the crude, which was purified using SiO2 flash chromatography (Biotage® Isolera™) with 10-50% of hexane/ethyl acetate to give rise to 23, (2.8 gm, 72%) as a yellow powder.

Synthesis of 24, tert-butyl 2-((9Z,12Z)-octadeca-9,12-dienamido)-3-(((9Z,12Z)-octadeca-9,12-dienoyl)(octadecyl)carbamoyl)-4,7-dihydrothieno[2,3-c]pyridine-6(5H)-carboxylate was synthesized using the general procedure A from linoleyl chloride (14d, 1495 mg, 5 mmol) [synthesized from linoleic acid (1402 mg, 5 mmol), 100 μL DMF, oxalyl chloride (513 μL, 6 mmol), and DCM (50 mL)], analog 23 (550 mg, 1 mmol), DMAP (5 mg, catalytic), triethylamine (1 mL, excess), and DCM (25 mL). The crude product was isolated on SiO₂ using 10-50% hexane/EA to give 648 mg (60%) of a yellow semi-solid.

Synthesis of 25, 2-((9Z,12Z)-octadeca-9,12-dienamido)-N-((9Z,12Z)-octadeca-9,12-dienoyl)-N-octadecyl-4,5,6,7-tetrahydrothieno[2,3-c]pyridine-3-carboxamide was synthesized using the general procedure B from 24 (600 mg, 0.56 mmol) and TFA:DCM (1:1, 6 mL). The resulting solution was diluted with DCM (40 mL), washed with 0.5 N NaOH (pH ˜12), and brine (30 mL), dried over MgSO₄, and concentrated under vacuum to yield 512 mg (94%) of a yellow oil.

### Synthesis of 26a-b:

26a, 6-(3-(dimethylamino)propanoyl)-2-((9Z,12Z)-octadeca-9,12-dienamido)-N-((9Z,12Z)-octadeca-9,12-dienoyl)-N-octadecyl-4,5,6,7-tetrahydrothieno[2,3-c]pyridine-3-: Using the general procedure C, with 25 (500 mg, 0.51 mmol), 3-(Dimethylamino)propionic acid hydrochloride (153 mg, 1 mmol), DIPEA (175 μl, 1 mmol), HATU (380 mg, 1 mmol), and DMF (8 mL), 26a was prepared as a yellow film, 212 mg (38%).

26b, 6-(4-(dimethylamino)butanoyl)-2-((9Z,12Z)-octadeca-9,12-dienamido)-N-((9Z,12Z)-octadeca-9,12-dienoyl)-N-octadecyl-4,5,6,7-tetrahydrothieno[2,3-c]pyridine-3-carboxamide: Using the general procedure C, with 25 (500 mg, 0.51 mmol), 4-(dimethylamino)butanoic acid hydrochloride (167 mg, 1 mmol), DIPEA (175 μl, 1 mmol), HATU (380 mg, 1 mmol), and DMF (8 mL), 26b was prepared as a yellow film, 188 mg (34%).

### Example 6

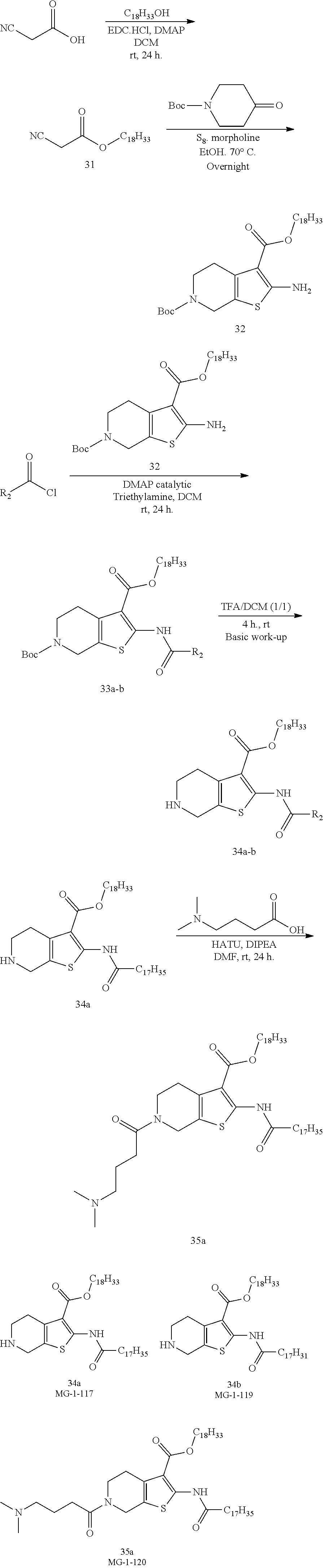

Synthesis of 31, (9Z,12Z)-octadeca-9,12-dien-1-yl 2-cyanoacetate: Using the general procedure D, with cyanoacetic acid (2.9 gm, 35 mmol), linoleyl alcohol (14 mL, 45.5 mmol), DMAP (280 mg, 2.3 mmol), EDC (8 gm, 42 mmol), and DCM (100 mL), 31 was prepared as a yellow powder, 9.3 gm (80%).

Synthesis of 32, 6-(tert-butyl) 3-((9Z,12Z)-octadeca-9,12-dien-1-yl) 2-amino-4,7-dihydrothieno[2,3-c]pyridine-3,6(5H)-dicarboxylate: A 250 mL one-neck round bottom flask was charged with 31 (1.6 gm, 4.8 mmol), tert-butyl 4-oxopiperidine-1-carboxylate (960 g, 4.8 mmol), sulfur (154 mg, 4.8 mmol), morpholine (1 mL, excess), and ethanol (25 mL). The mixture was stirred at 70° C. for 12 hours. The reaction mixture was cooled, and the solvent was evaporated under reduced pressure to give rise to the crude, which was purified using SiO₂ flash chromatography (Biotage® Isolera™) with 6-25% of hexane/ethyl acetate to give rise to 32, (2.2 gm, 84%) as a yellow oil.

### Synthesis of 33a-b:

33a, 6-(tert-butyl) 3-((9Z,12Z)-octadeca-9,12-dien-1-yl) 2-stearamido-4,7-dihydrothieno[2,3-c]pyridine-3,6(5H)-dicarboxylate was synthesized using the general procedure A from stearoyl chloride (606 mg, 2 mmol) [synthesized from stearic acid (569 mg, 2.0 mmol), 100 μL DMF, oxalyl chloride (205 μL, 2.4 mmol), and DCM (10 mL)], analog 32 (547 mg, mmol), DMAP (5 mg, catalytic), triethylamine (1 mL, excess), and DCM (13 mL). The crude product was purified using SiO₂ flash chromatography (Biotage® Isolera™) with 6-25% of hexane/ethyl acetate to give rise to 33a, (340 mg, 42%) as a yellow powder.

33b, 6-(tert-butyl) 3-((9Z,12Z)-octadeca-9,12-dien-1-yl) 2-((9Z,12Z)-octadeca-9,12-dienamido)-4,7-dihydrothieno[2,3-c]pyridine-3,6(5H)-dicarboxylate was synthesized using the general procedure A from linoleyl chloride (14d, 598 mg, 2 mmol) [synthesized from linoleic acid (561 mg, 2.0 mmol), 100 μL DMF, oxalyl chloride (205 μL, 2.4 mmol), and DCM (10 mL)], analog 32 (547 mg, 1 mmol), DMAP (5 mg, catalytic), triethylamine (1 mL, excess), and DCM (13 mL). The crude product was purified using SiO₂ flash chromatography (Biotage® Isolera™) with 6-25% of hexane/ethyl acetate to give rise to 33b, (356 mg, 44%) as a yellow oil.

### Synthesis of 34a-b:

34a, (9Z,12Z)-octadeca-9,12-dien-1-yl 2-stearamido-4,5,6,7-tetrahydrothieno[2,3-c]pyridine-3-carboxylate was synthesized using the general procedure B from 33a (713 mg, 1 mmol) and TFA:DCM (1:1, 6 mL). The resulting solution was diluted with DCM (20 mL), washed with 0.5 N NaOH (pH ˜12), and brine (50 mL), dried over MgSO₄, and concentrated under vacuum to yield 600 mg (84%) of a yellow powder.

34b, (9Z,12Z)-octadeca-9,12-dien-1-yl 2-((9Z,12Z)-octadeca-9,12-dienamido)-4,5,6,7-tetrahydrothieno[2,3-c]pyridine-3-carboxylate was synthesized using the general procedure B from 33b (709 mg, 1 mmol) and TFA:DCM (1:1, 6 mL). The resulting solution was diluted with DCM (20 mL), washed with 0.5 N NaOH (pH ˜12), and brine (50 mL), dried over MgSO4, and concentrated under vacuum to yield 612 mg (86%) of a yellow oil.

Synthesis of 35a, (9Z,12Z)-octadeca-9,12-dien-1-yl 6-(4-(dimethylamino)butanoyl)-2-((9Z,12Z)-octadeca-9,12-dienamido)-4,5,6,7-tetrahydrothieno[2,3-c]pyridine-3-carboxylate: Using the general procedure C, with 34a (214 mg, 0.3 mmol), 4-(dimethylamino)butanoic acid hydrochloride (167 mg, 1 mmol), DIPEA (175 μl, 1 mmol), HATU (380 mg, 1 mmol), and DMF (8 mL), 35a was prepared as a yellow powder, 106 mg (43%).

### Example 7

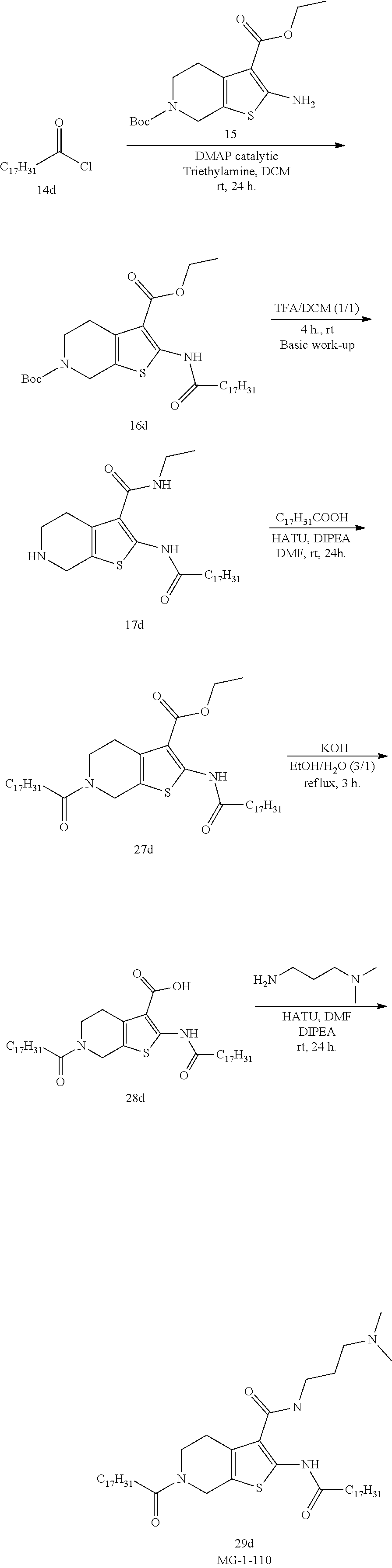

Synthesis of 27d, ethyl 2-((9Z,12Z)-octadeca-9,12-dienamido)-6-((9Z,12Z)-octadeca-9,12-dienoyl)-4,5,6,7-tetrahydrothieno[2,3-c]pyridine-3-carboxylate: Using the general procedure C, with 17d (976 mg, 2 mmol), linoleic acid (1.24 mL, 4 mmol), DIPEA (700 μl, 4 mmol), HATU (1.52 gm, 4 mmol), and DMF (12 mL), 27d was prepared as a white semi-solid, 1.1 gm (73%).

Synthesis of 28d, 2-((9Z,12Z)-octadeca-9,12-dienamido)-6-((9Z,12Z)-octadeca-9,12-dienoyl)-4,5,6,7-tetrahydrothieno[2,3-c]pyridine-3-carboxylic acid: An oven-dried 100 mL round bottom flask was charged with 27d (1.1 gm, 1.46 mmol) and ethanol (12 mL). Then a 10% solution of potassium hydroxide in water (4.5 mL, 8 mmol) was added, and the reaction was allowed to stir for an additional 15 minutes at room temperature. The reaction mixture was then refluxed for 3 hours. The progress was monitored using TLC. After completion, the reaction was allowed to be cooled, and 1 N HCl (pH˜3-4), DCM (25 mL), and water (25 mL) were added. The organic phase was separated and washed with 3×20 mL water, followed by brine. The crude was dried over sodium sulfate and purified using SiO₂ flash chromatography (Biotage® Isolera™) with 10-50% of hexane/ethyl acetate to give rise to 28d (308 mg, 29%) as a light-yellow powder.

Synthesis of 29d, N-(3-(dimethylamino)propyl)-2-((9Z,12Z)-octadeca-9,12-dienamido)-6-((9Z,12Z)-octadeca-9,12-dienoyl)-4,5,6,7-tetrahydrothieno[2,3-c]pyridine-3-carboxamide: Using the general procedure C, with 28d (140 mg, 0.2 mmol), N¹,N¹-dimethylpropane-1,3-diamine (60 μl, 0.5 mmol), DIPEA (70 μl, 0.4 mmol), HATU (152 mg, 0.4 mmol), and DMF (4 mL), 29d was prepared as a colorless oil, 28 mg (17%).

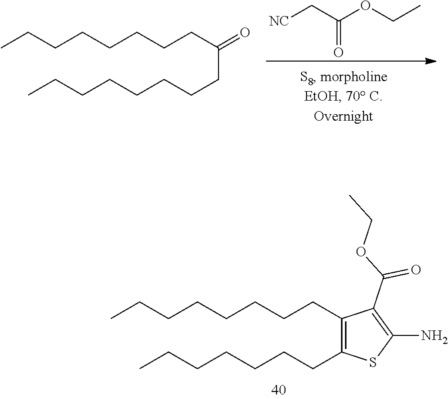

Synthesis of 40, ethyl 2-amino-5-heptyl-4-octylthiophene-3-carboxylate: A 250 mL one-neck round bottom flask was charged with 9-heptadecanone (3.2 gm, 12.4 mmol), ethyl cyanoacetate (1.44 mL, 13.6 mmol), sulfur (0.44 gm, 13.6 mmol), morpholine (3 mL, excess), and ethanol (25 mL). The mixture was stirred at 70° C. for 12 hours. The reaction mixture was cooled, and the solvent was evaporated under reduced pressure to give rise to the crude, which was purified using SiO₂ flash chromatography (Biotage® Isolera™) with 6-25% of hexane/ethyl acetate to give rise to 40, (3.6 gm, 76%) as a yellow crystalline powder.

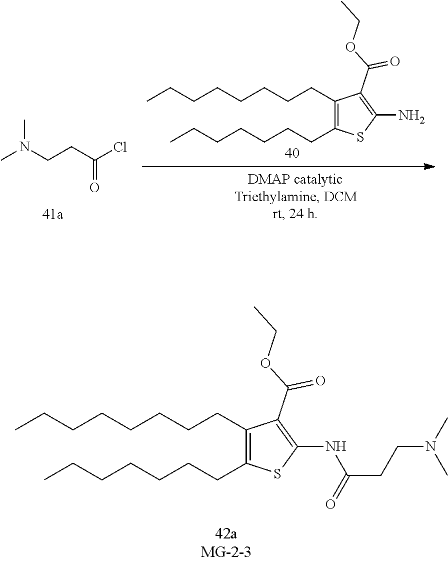

### Synthesis of 42a-b:

42a, ethyl 2-(3-(dimethylamino)propanamido)-5-heptyl-4-octylthiophene-3-carboxylate was synthesized using the general procedure A from 3-(dimethylamino)propanoyl chloride (272 mg, 2 mmol) [synthesized from 3-(dimethylamino)propanoic acid hydrochloride (307 mg, 2.0 mmol), 100 μL DMF, oxalyl chloride (205 μL, 2.4 mmol), and DCM (10 mL)], analog 40 (198 mg, 0.5 mmol), DMAP (5 mg, catalytic), triethylamine (1 mL, excess), and DCM (13 mL). The crude was purified using SiO₂ flash chromatography (Biotage® Isolera™) using 1-9% methanol/DCM (1% of 7 N NH₃ additive) to obtain the desired product 42a, (88 mg, 36%) as a yellow powder.

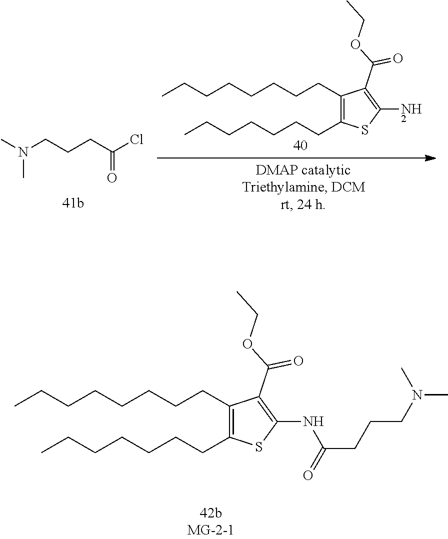

42b, ethyl 2-(4-(dimethylamino)butanamido)-5-heptyl-4-octylthiophene-3-carboxylate was synthesized using the general procedure A from 4-(dimethylamino)butanoyl chloride (299 mg, 2 mmol) [synthesized from 4-(dimethylamino)butanoic acid hydrochloride (335 mg, 2.0 mmol), 100 μL DMF, oxalyl chloride (205 μL, 2.4 mmol), and DCM (10 mL)], analog 40 (198 mg, 0.5 mmol), DMAP (5 mg, catalytic), triethylamine (1 mL, excess), and DCM (13 mL). The crude was purified using SiO₂ flash chromatography (Biotage® Isolera™) using 1-9% methanol/DCM (1% of 7 N NH₃ additive) to obtain the desired product 42b, (98 mg, 39%) as a yellow powder.

### Example 9

### Alternative Key Intermediate Syntheses

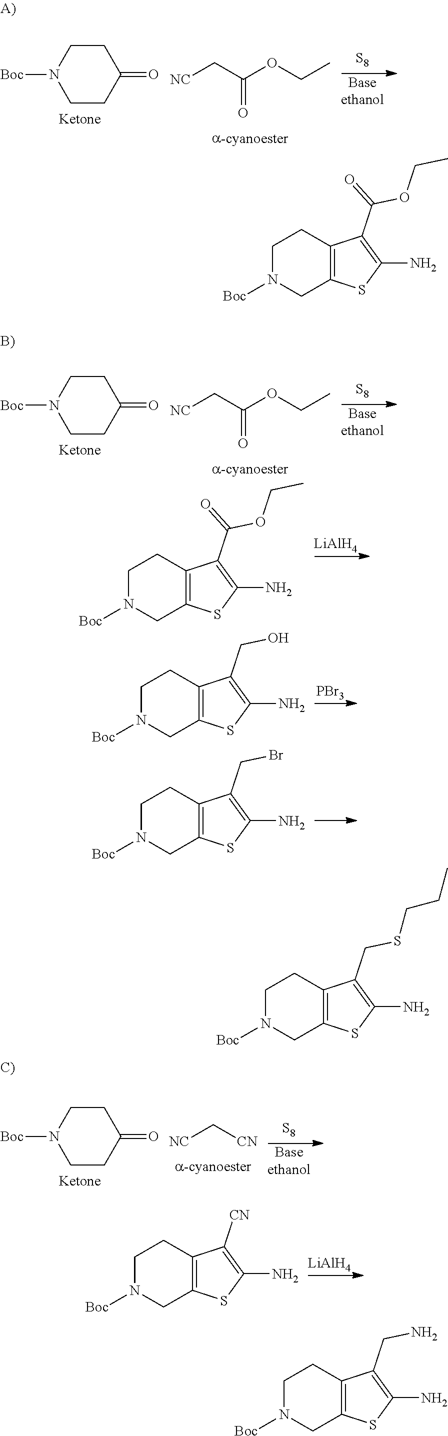

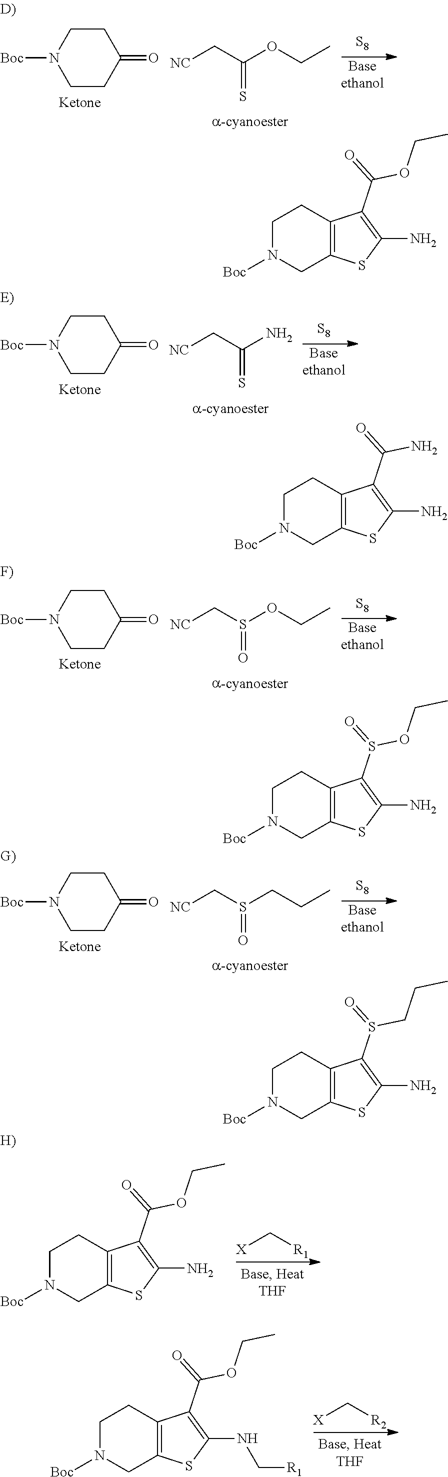

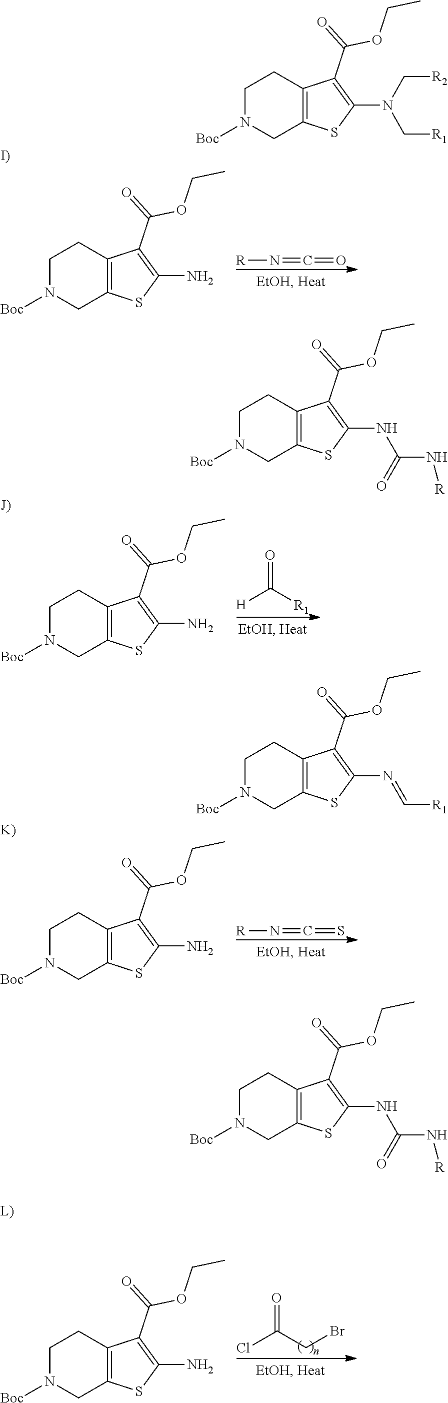

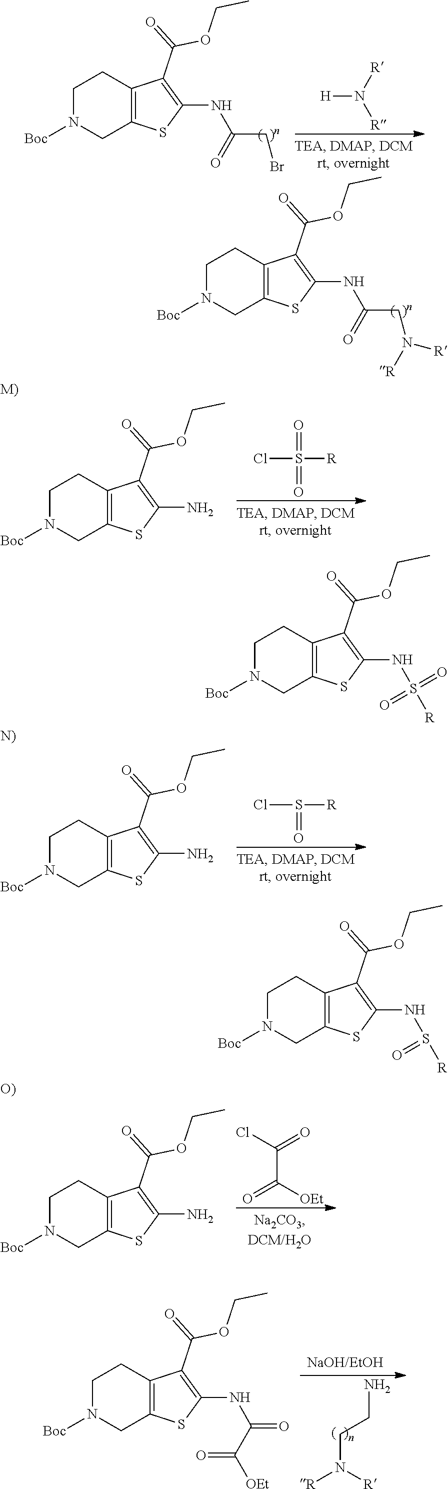

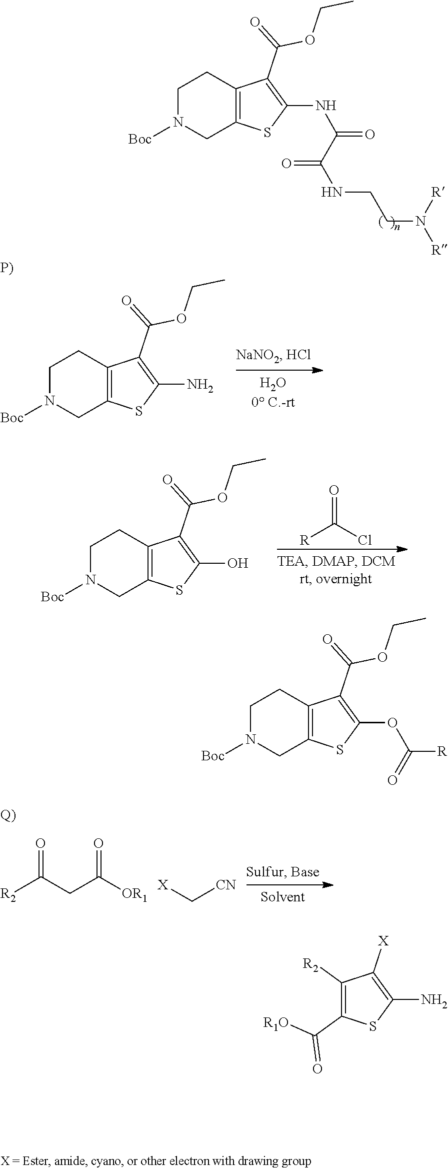

Additional information concerning synthesizing thiophene compounds can be found in McKibben, B. P.; Cartwright, C. H.; Castelhano, A. L. Practical synthesis of tetrasubstituted thiophenes for use in compound libraries. Tetrahedron Letters 1999, 40 (30), 5471, which is incorporated herein by reference.

### Example 10

### Further Additional Syntheses

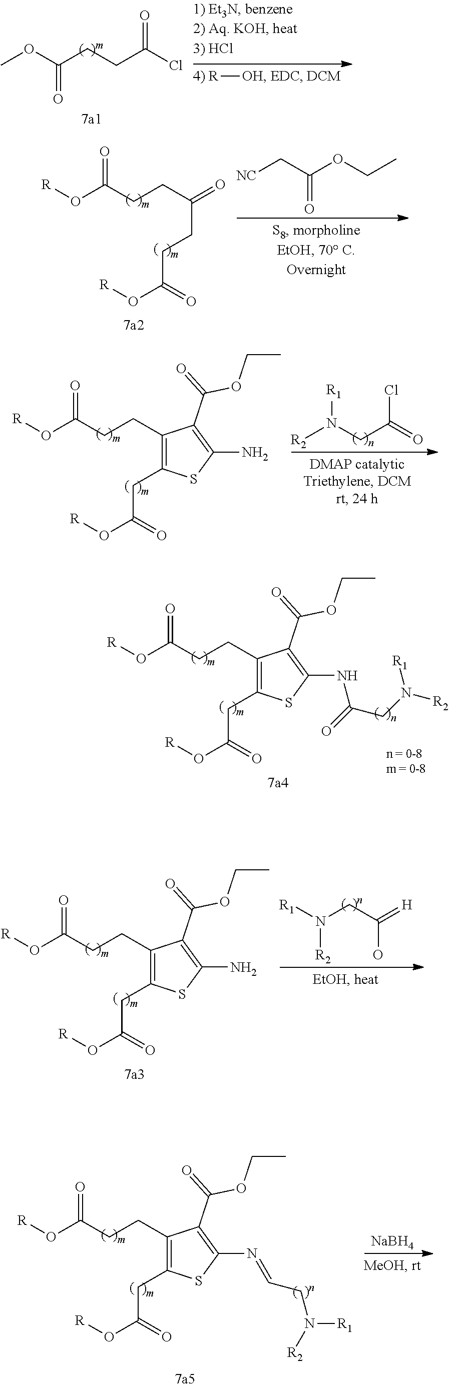

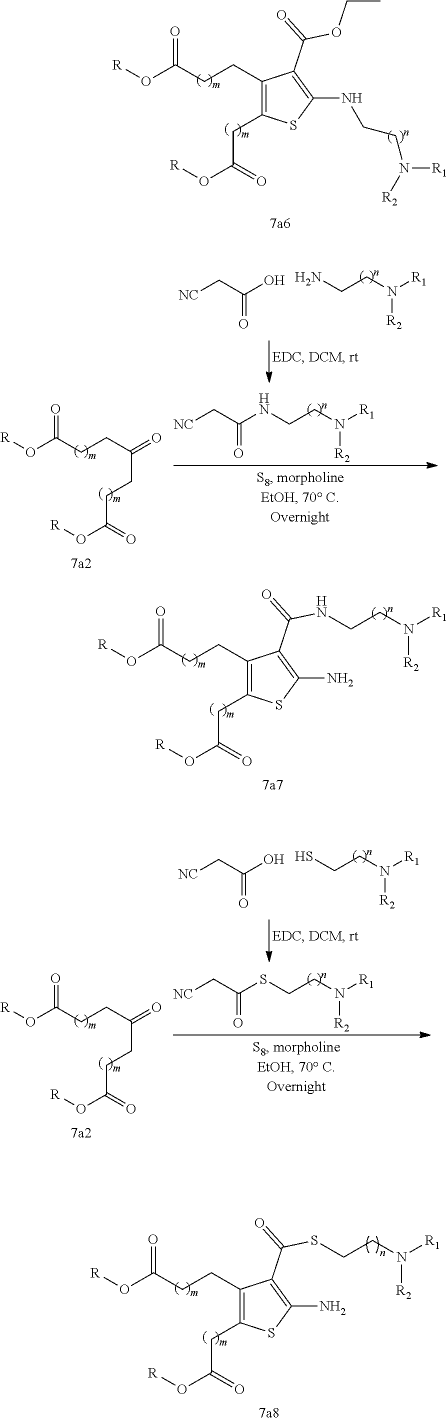

Additional information concerning synthesizing thiophene compounds 7a1-7a2 can be found in Durham, L. J., 1963. D, J. McLeod, and J. Cason,”. Org. Synth. Coll, 4, p. 635, which is incorporated herein by reference.

### Representative Compounds Include:

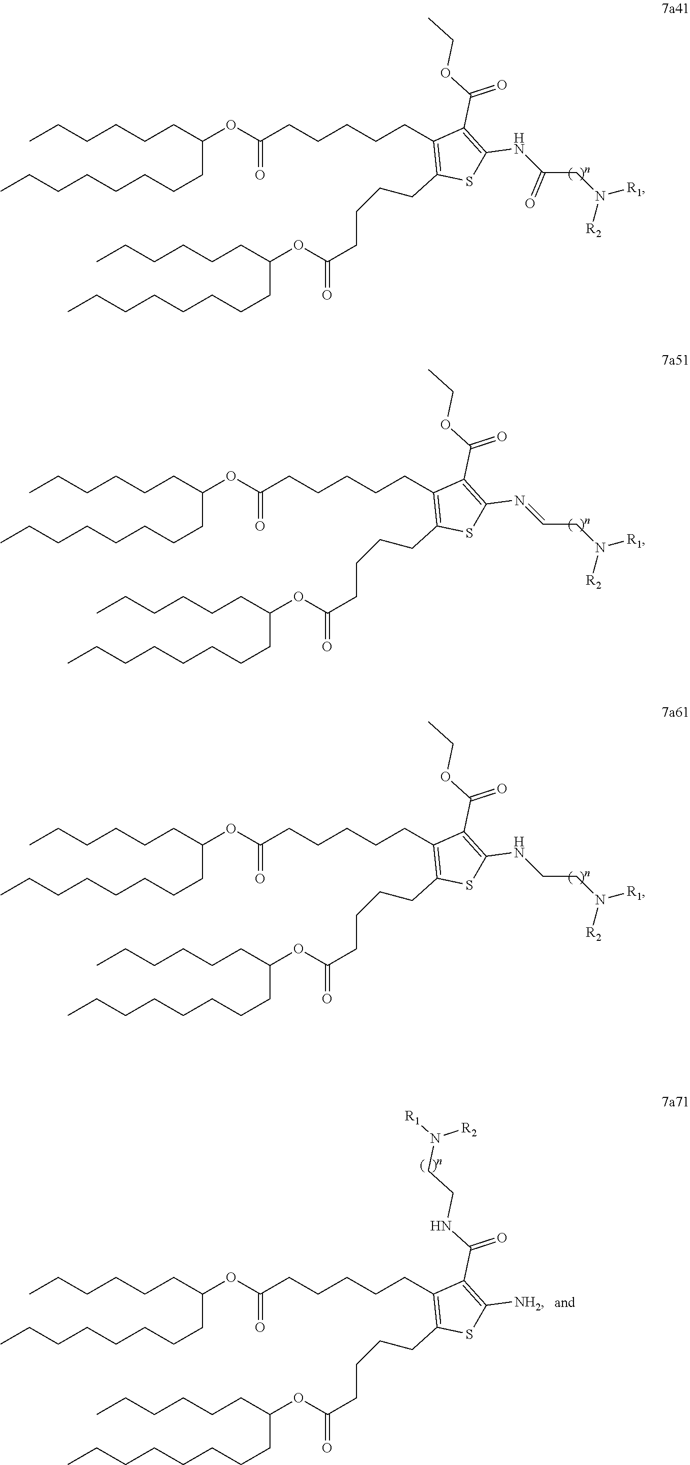

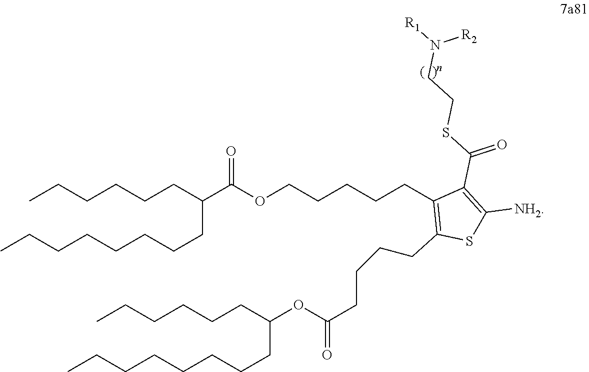

### Example 11

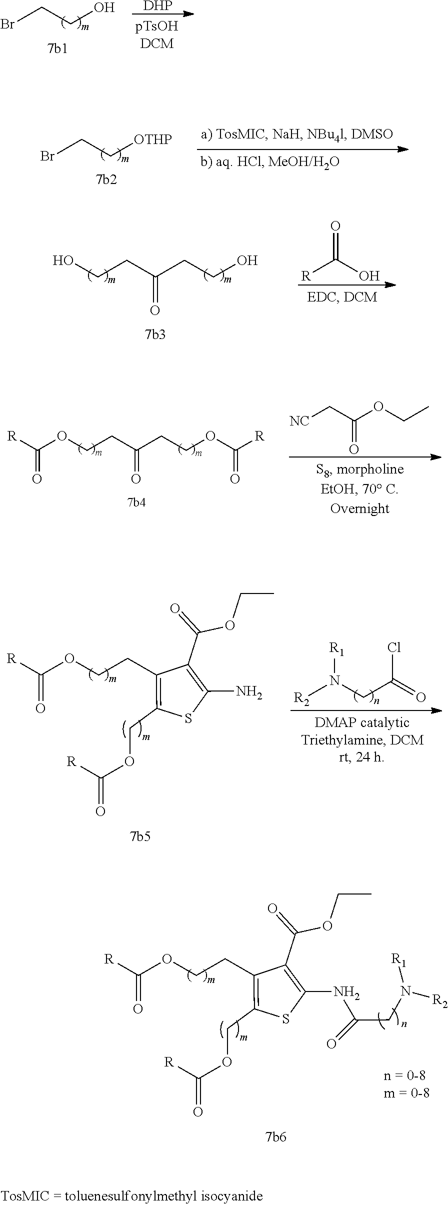

Additional information concerning synthesizing thiophene compounds 7b1-7b3 can be found in Mueller, R., Yang, J., Duan, C., Pop, E., Geoffroy, O. J., Zhang, L. H., Huang, T. B., Denisenko, S., McCosar, B. H., Oniciu, D. C. and Bisgaier, C. L., 2004, Long hydrocarbon chain keto diols and diacids that favorably alter lipid disorders in vivo. Journal of medicinal chemistry, 47(24), pp. 6082-6099, which is incorporated herein by reference.

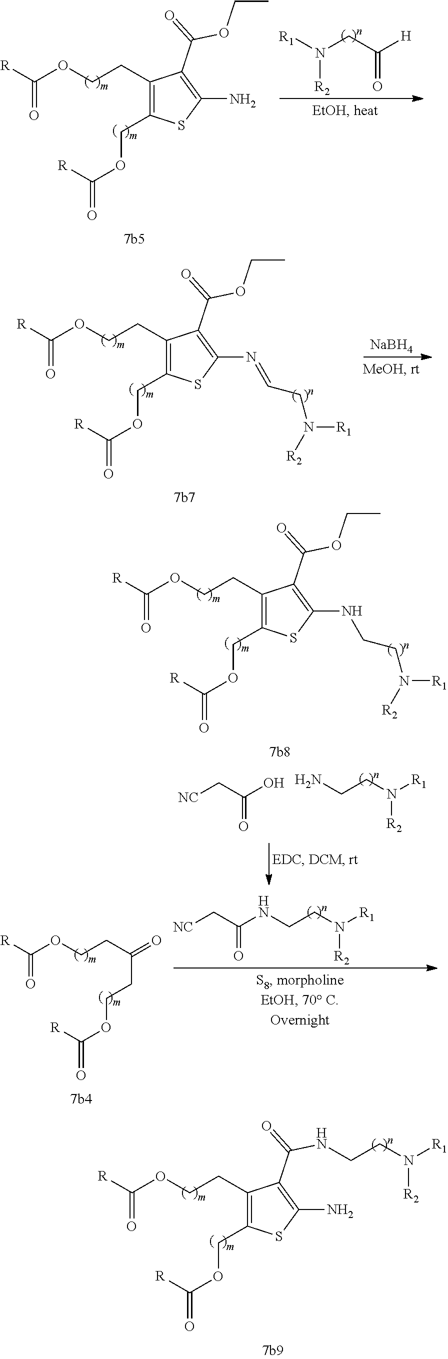

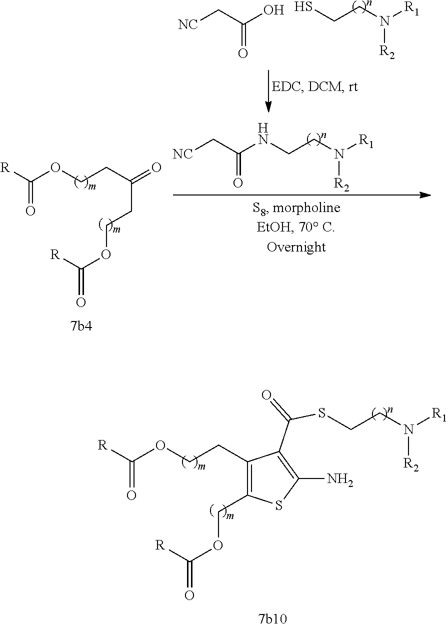

### Representative Examples Include:

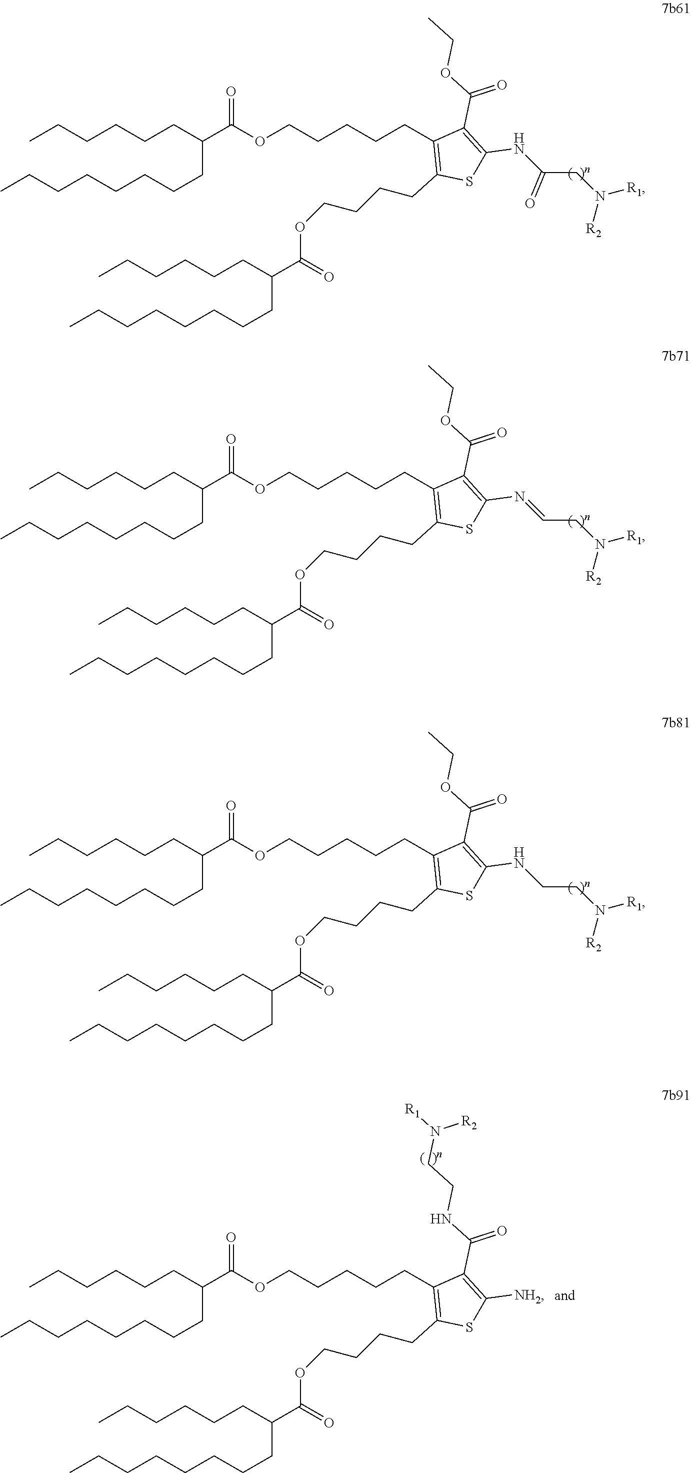

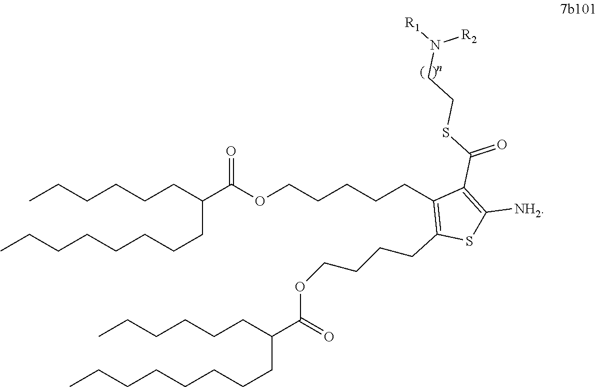

### Example 12

### Nanoparticle Preparation

Lipid nanoparticles were prepared by microfluidic mixing. Briefly, a lipid solution in ethanol (containing MG-lipid, phospholipid, a sterol, and DMG-PEG2000 at a total concentration of 5.5 mM and 20-fold excess by mass compared to mRNA) was combined with an aqueous mix containing mRNA, water, and 100 mM pH 4 citrate buffer (3-fold excess by volume compared to lipid solution) using a Nanoassemblr Spark or Benchtop microfluidic mixer. Nanoparticles were then dialyzed twice against 3 L of pH 7 1×PBS buffer for 4 hours and overnight, respectively, and concentrated using centrifuge filters. Nanoparticle size was characterized using dynamic light scattering (DLS) for 0.3-0.5 v/v % particle solutions in PBS, and encapsulation efficiency and mRNA concentration for further studies was characterized using Invitrogen Quant-iT RiboGreen assay.

In certain examples, LNPs containing various ionizable lipids (such as a lipid disclosed herein), DSPC, cholesterol, DMG-PEG-2000, and FLuc mRNA were prepared using a standard microfluidic mixing procedure at a total flow rate of 9-12 ml/min, such as about 9, 10, 11, or 12. In certain examples, the molar ratio between components was 50:10:38.5:1.5 (disclosed lipid:DSPC:cholesterol:DMG-PEG-2000, respectively); however, formulations have also been tested with various phospholipids (DOPE in place of DSPC), sterols (beta-sitosterol, campesterol, fucosterol, or stigmastanol in place of cholesterol), and amounts of DMG-PEG-2000 (e.g., molar ratios between components 50:7:38.5:4.5). The quantity of the lipids needed may be determined by the amount of mRNA needed for the experiments. In certain embodiments, the molar ratio between the ionizable lipid and mRNA, also known as N/P ratio, is ˜5.5:1, respectively; however, formulations have also been tested with N/P ratio of ˜10.6:1. In certain examples, volume ratio between organic (lipid) and aqueous (mRNA) phases was kept constant at 1:3, respectively.

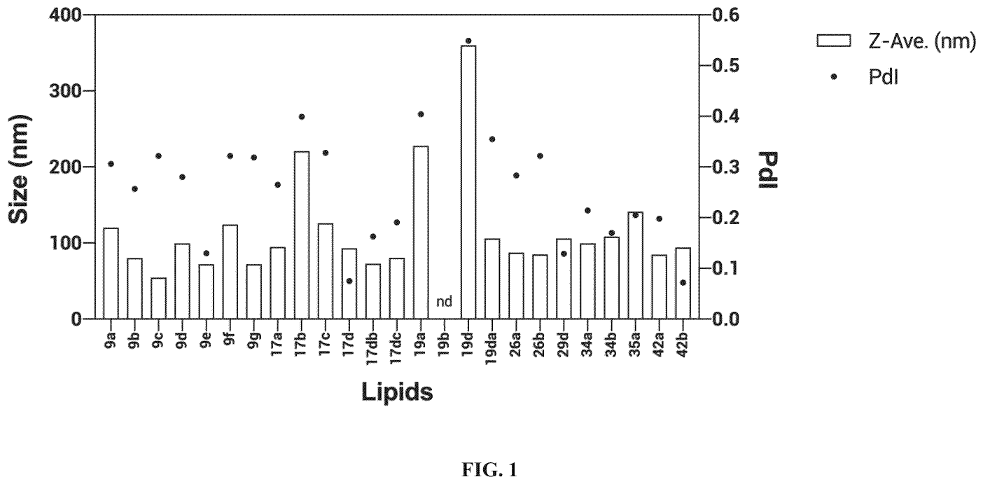

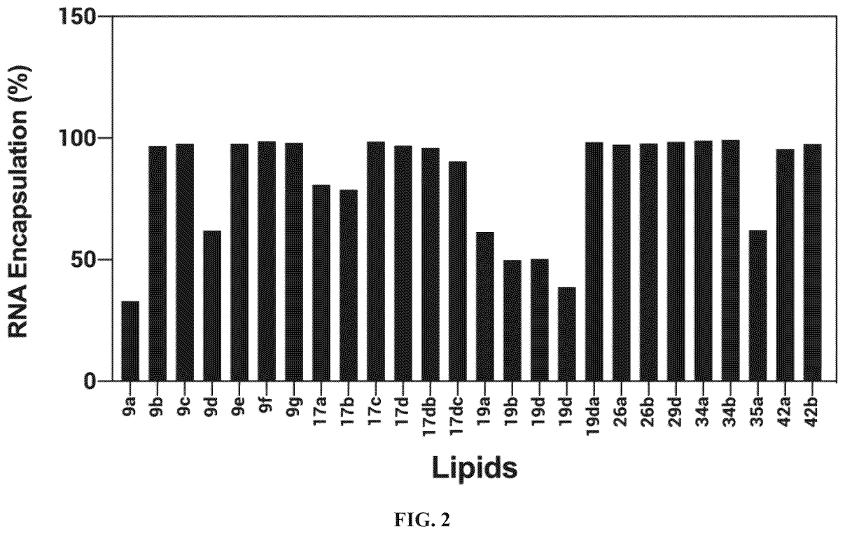

In certain examples, the size of LNPs was 60-120 nm with a moderate polydispersity (<0.4) as measured by DLS (see FIG. 1). Encapsulation efficiency in most cases was over 90% (FIG. 2). These measurements served as preliminary classifiers to evaluate the feasibility of in vitro and in vivo studies (e.g., the particle size of >200 nm and encapsulation efficiency of <60% were disqualified from in vivo experiments).

TABLE 2
An exemplary lipid composition of LNP and R-LNPs by DLS. Lipid component and
amount of lipids used to formulate lipid nanoparticles are described herein.

| Composition | LNP | LNPa | LNPx | eLNPx | LNPz |
|---|---|---|---|---|---|
| MC3 or MG lipid (%) | 50 | 50 | 50 | 50 | 50 |
| DSPC (%) | 10 | 10 | 10 | 10 | 10 |
| Cholesterol (%) | 38.5 | 38.5 | 38.5 | 0 | 38.5 |
| β-Sitosterol (%) | 0 | 0 | 0 | 38.5 | 0 |
| DMG-PEG (%) | 1.2 | 1.2 | 1.2 | 1.2 | 1.2 |
| DSPE-PEG-2k-Carboxy NHS (%) | 0 | 0 | 0.3 | 0.3 | 0 |
| DSPE-PEG-2k-Carboxylic Acid (%) | 0 | 0 | 0 | 0 | 0.3 |
| DSPE-PEG-2k-Amine (%) | 0 | 0.3 | 0 | 0 | 0 |

### Example 13

### In Vitro Transfection

Mammalian cell lines used to measure cytotoxicity and transfection efficiency include HeLa (human cervical epithelial cells), HEK293T/17 (human embryonic kidney cells), HepG2 (human liver epithelial-like cells), and Jurkat E6-1 (human T-lymphocytes). The cells were seeded at 4000 cells/well in a 96-well plate and incubated for 24 hours. The next day, the LNPs of interest were diluted with 1×PBS to 50 ng/ul and administered at predetermined doses to the cells. The cell viability and transfection efficiency were evaluated on the following day (after 24-hour incubation of cells with LNPs). The cell viability was measured with Promega CellTiter-Fluor Cell Viability Assay, and the transfection efficiency was measured with Promega ONE-Glo Luciferase Assay System using Tecan Infinite M200 PRO multimode plate reader instrument. The dose range varied from 50 to 200 ng of nucleic acid per well.

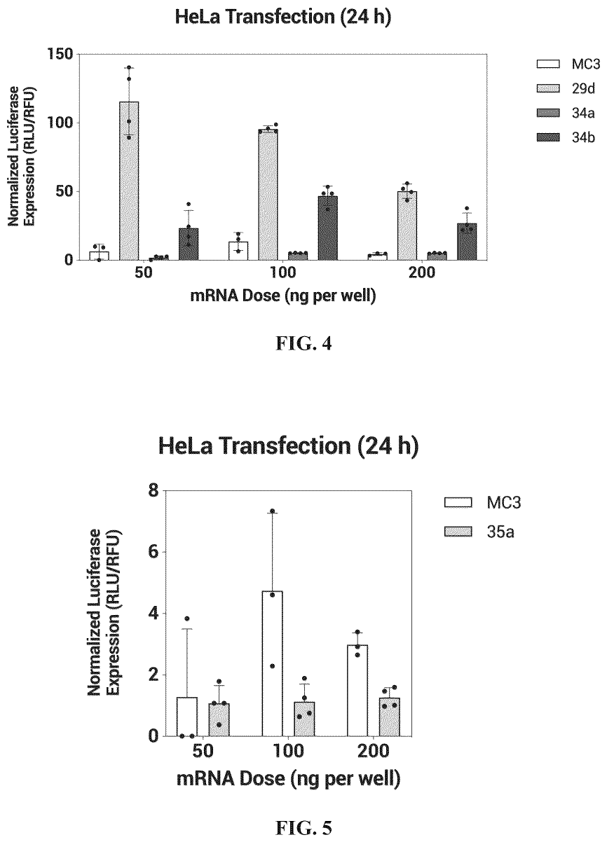

Lipids 19da, 26a, 26b, 29d, 34a, 34b, and 35a all demonstrated favorable transfection profile and cytotoxicity in HeLa (FIGS. 3 to 5) when compared to negative control (PBS; data not shown) and positive control (MC3 LNPs at the same dose). Cell viability in these cell lines was >60% (data not shown). For LNPs with lipids 19da, 26a, and 26b, transfection efficiency in HeLa was greater than positive control up to 5 times and greater at 200 ng/well than at 50 ng/well, indicating a dose response (FIG. 3). Similarly, LNPs with lipids 29d, 34a, and 34b demonstrated up to 12× increase in transfection efficiency in HeLa (FIG. 4). Interestingly, LNPs with lipid 29d indicated inverse dose response that could not be attributed to cytotoxicity. Lastly, LNPs with lipid 35a were able to successfully transfect HeLa cells but not to the extent of the positive control at the same dose (up to 4×fold decrease in transfection efficiency; FIG. 5). Lipid 19da showed almost 5-fold improvement compared to the positive control in the HeLa cell line at 200 ng treatment, yet 3-fold decline in HEK293T/17 and HepG2 cell lines at an identical dose. Similar observations were made for lipids 29d, 34a, 34b, and 35a in HEK293T/17 (data not shown).

### Example 14

### In Vivo Formulation Studies

A nanoparticle formulation including firefly luciferase (Fluc) mRNA was administered to BALB/c mice by intravenous, intramuscular, intravitreal, or subretinal injection. The intravenous injection was performed by tail-vein or retro-orbital injection. Mice that received a dose of nanoparticle formulation were housed until bioluminescence imaging. Bioluminescence imaging is a method to quantify luminescent signals from the subject. Prior to bioluminescence imaging, the mice were sedated with inhalation of isoflurane. The sedated mice received a dose of D-luciferin salt, which is a substrate of firefly luciferase, by intraperitoneal injection at 150 mg/kg 10 minutes prior to bioluminescence imaging. Bioluminescence imaging was performed on the mice with various exposure times ranging from 1 to 300 seconds using IVIS Lumina XRMS from PerkinElmer. To evaluate the protein expression at an organ level, internal organs of a mouse, such as the liver, lungs, kidneys, spleen, lymph nodes, and heart, and external tissues of mouse, such as muscle tissues close to a site of injection, were excised.

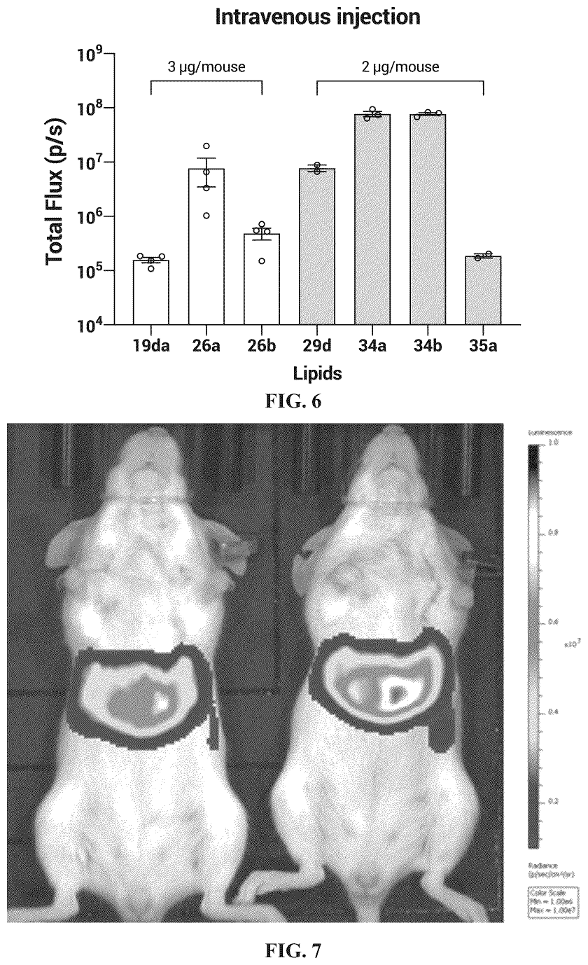

The results of the in vivo studies for various LNPs administered via several important administration routes are shown in FIGS. 6 to 12.

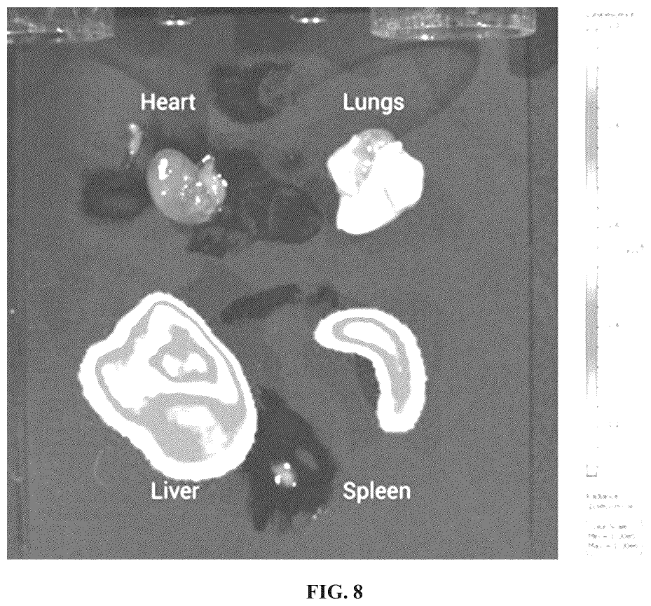

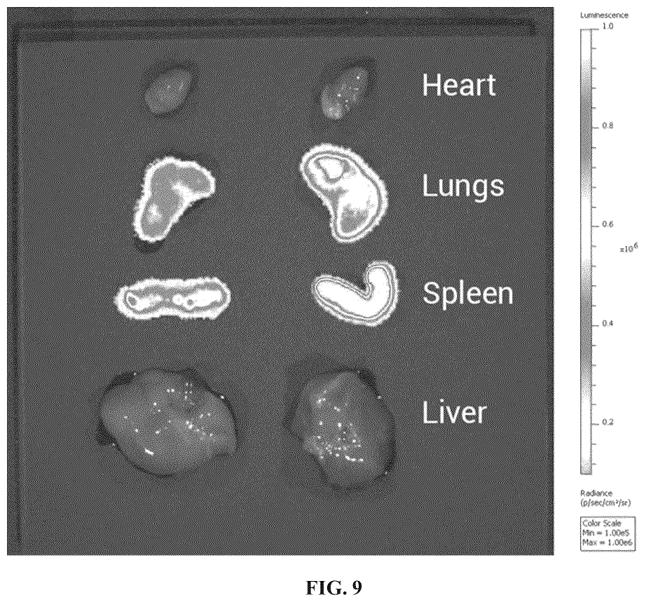

As previously described in the In vitro transfection section (Example 13), lipids 19da, 26a, 26b, 29d, 34a, 34b, and 35a showed potential for translating in vitro results into animal models. Intravenous administration of LNP formulations containing these lipids to mice produced luminescent signals detected by in vivo imaging, indicating successful transfection (FIG. 6). The degree of transfection varied for different formulations, with LNPs containing lipid 34a and 34b producing the highest light flux indicative of the highest extent of transfection in the tested series. The pattern of luminescence was consistent with transfection of liver and spleen (in vivo and ex vivo data for lipid 34a shown in FIGS. 7 and 8, respectively). Upon further investigation of extracted organs ex vivo for lipid 29d, the luminescence signal was found to have originated from the spleen and lungs (FIG. 9). Even though LNPs with lipid 29d also produced a luminescence pattern consistent with liver transfection, the discrepancy in the liver luminescence attribution may be related to the ex vivo sample preparation (namely, rinsing an excised organ with PBS), which suggests a superficial level of liver transfection. This discovery has a tremendous potential for the delivery of mRNA to lung tissues from the systemic circulation, a hard-to-reach therapeutic target that was previously only accessed by local administration.

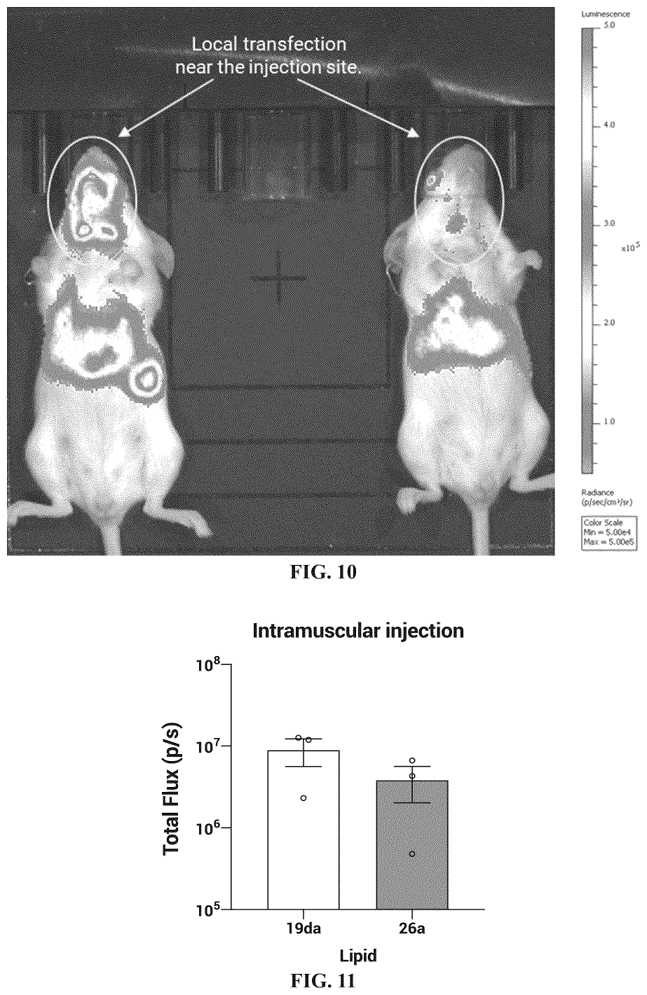

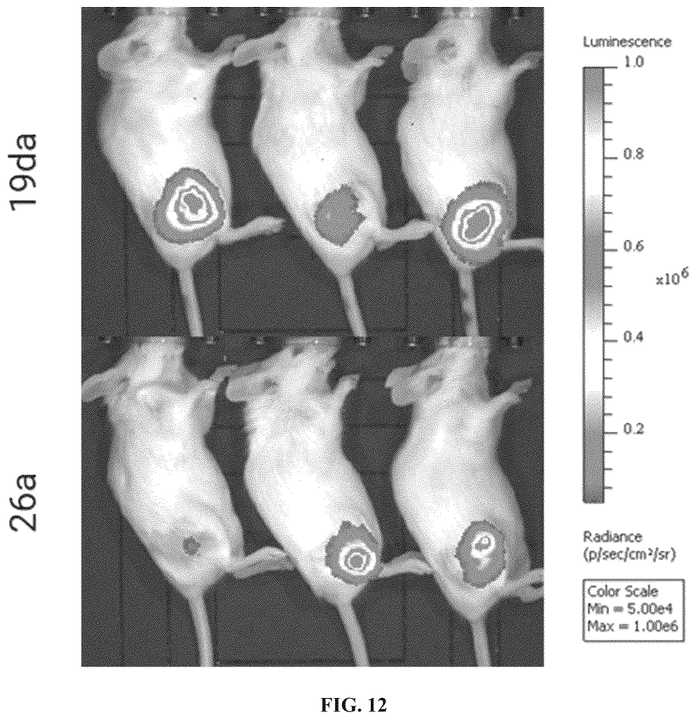

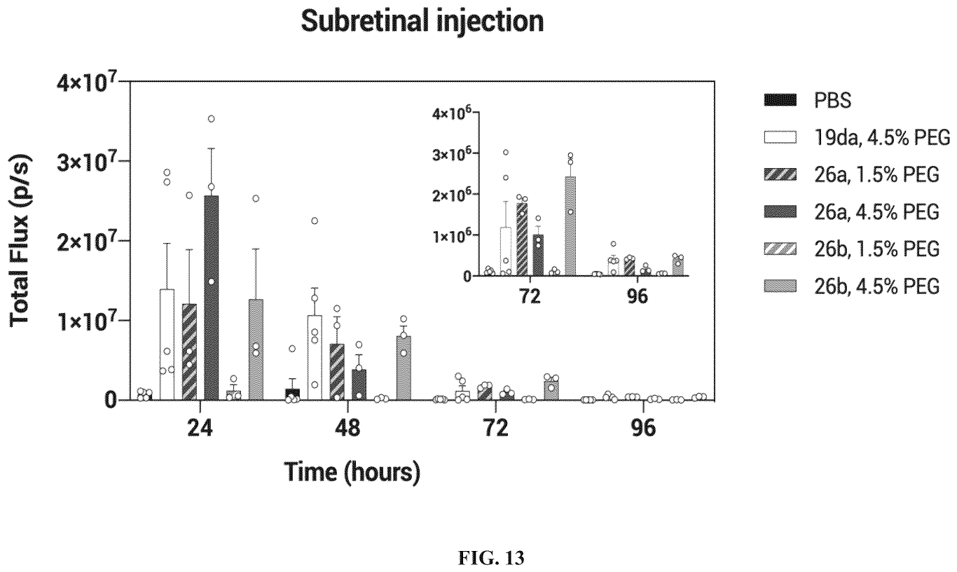

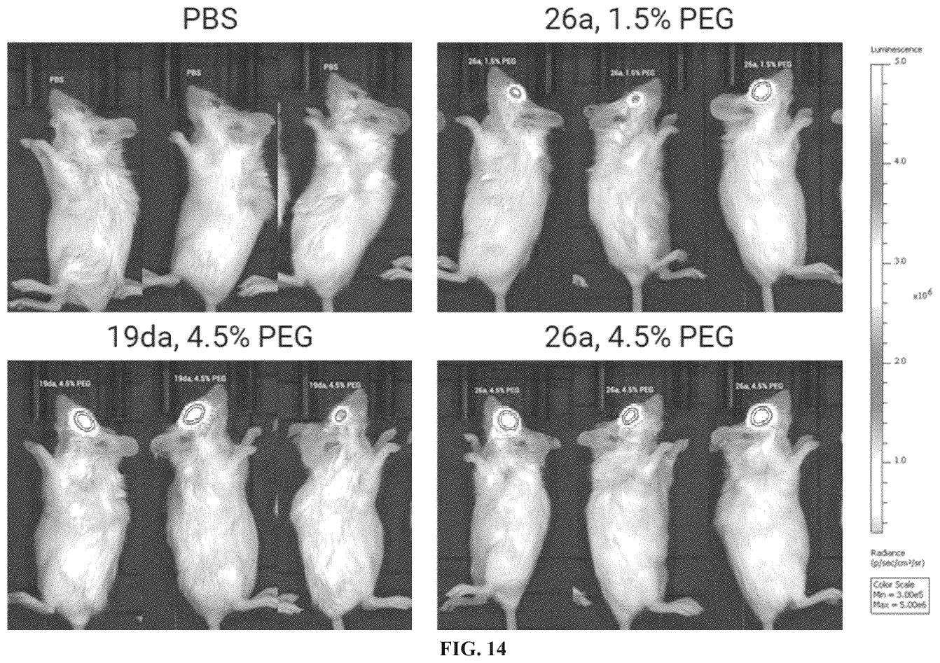

Analysis of transfected issues for lipids 19da, 26b, and 26a revealed the luminescence pattern consistent with local transfection (FIG. 10). This finding suggested that these lipids may have a potential for successful delivery via other administration routes relying on direct injection into an organ and/or tissues as opposed to the selective organ uptake from systemic circulation. Therefore, those LNPs were also administered intramuscularly and subretinally. Intramuscular administration of LNPs containing lipids 19da and 26a resulted in successful and comparable transfection of local tissues when administered to the flanks of mice (FIGS. 11 and 12). Subretinal administration of LNPs containing lipids 19da, 26a, and 26b in “normal” (molar ratio of 50:10:38.5:1.5 as described above) and in highly PEGylated formulations (molar ratio of 50:7:38.5:4.5; the formulations are labeled as 19da 4.5% PEG, 26b 4.5% PEG, and 26a 4.5% PEG) have also produced greater significant light flux when compared to the negative control (FIGS. 13 and 14). The highly PEGylated formulations were included in this study due to the previously reported effects of PEG on transfection in the eye. Indeed, the incorporation of higher quantities of PEG-lipid appears to assist transfection when administered subretinally, as evident from higher light flux values for 26b 4.5% PEG compared to 26b 1.5% PEG, and 26a 4.5% PEG compared to 26a 1.5% PEG. In these series, LNPs 26a 4.5% PEG showed the highest transfection efficiency at 24-hour timepoint although the decay of the transfection efficiency over time was also more significant compared to 19da 4.5% PEG and 26b 4.5% PEG (similar highly PEGylated formulations) and to 26a 1.5% PEG (“normal” formulation). Any luciferase signal was not detected in mice that received LNPs with lipid 26b in a “normal” formulation. The transfection was the most prominent for all LNPs at a 24-hour time point and still detectable for LNPs 19da 4.5% PEG, 26b 4.5% PEG, and 26a 1.5% PEG 96 hours after the initial injection. These findings confirm the potentials of these novel lipids for gene delivery in cases where local administration is appropriate.

### Example 15

Inherited retinal diseases (IRDs) are a rare and heterogeneous group of neurodegenerative disorders that result in the loss or dysfunction of photoreceptor cells. The progressive loss of cells may lead to blindness. IRDs affect approximately one in every 2000 people worldwide, according to estimates. IRDs are linked to nearly 300 genes. The majority of IRD affects the photoreceptors (PRs), but other types can also affect the RPE or the inner retina. LNPs have emerged as a versatile platform for efficient gene delivery that reduces off-target risks when compared to viral formulations. Changes in the lipid-polyethylene glycols (PEGs) composition of LNPs have been investigated to achieve ocular gene delivery, but transfection is still limited to the RPE. It has been reported that LNPs have a very limited ability to deliver mRNA in PRs cells. A PEG molecular weight of 2 kDa or higher has been shown to sterically stabilize nanoparticles and reduce protein absorption. It has been demonstrated that intravitreal injection of cationic nanoparticles causes particle aggregation in the vitreous and that the particles do not spread far from the injection site. Interestingly, anionic (<200 nm) and PEGylated (2 kDa) particles were able to diffuse away from the needle track, indicating anionic particle diffusion ability and PEGylation enhancing the diffusion process. The preceding study also revealed that larger anionic particles (>500 nm) were significantly hampered and excessively aggregated to the injection site.

It has been demonstrated that subretinal injection of lipid-PEG modified LNPs resulted in cell-specific protein expression in the RPE. Similarly, intravitreal injection of these particles transfected RPE, Müller cells, the optic nerve head, and the trabecular meshwork. It was hypothesized that negative charged LNPs might have a varied distribution within the neural retina. Different anionic or cationic functional lipid-PEGs test the neural retina penetration ability of new LNP variants. Surface modification of LNPs with carboxy lipid-PEGs resulted in successful delivery to neural retina in a Ai9 mice. According to the findings, carboxylic group modified highly anionic particles outperformed less anionic particles in terms of PRs targeting.

### Design and Characterization of Novel LNP Variants

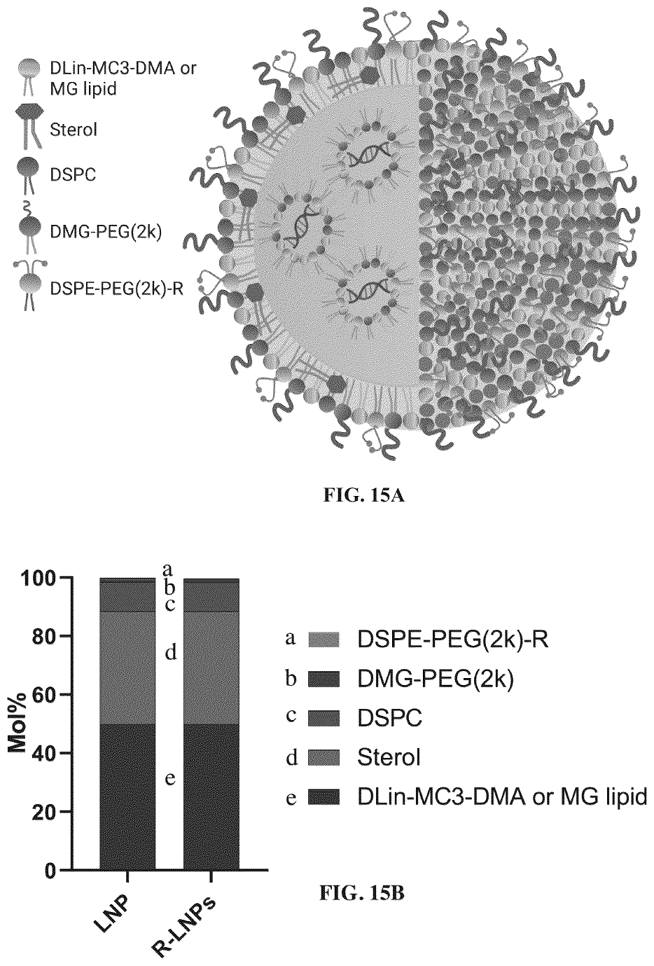

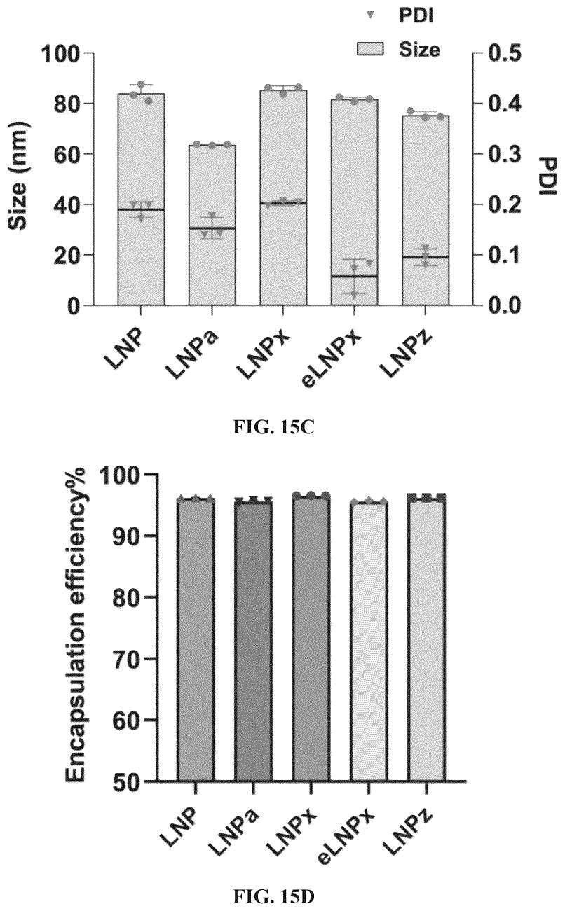

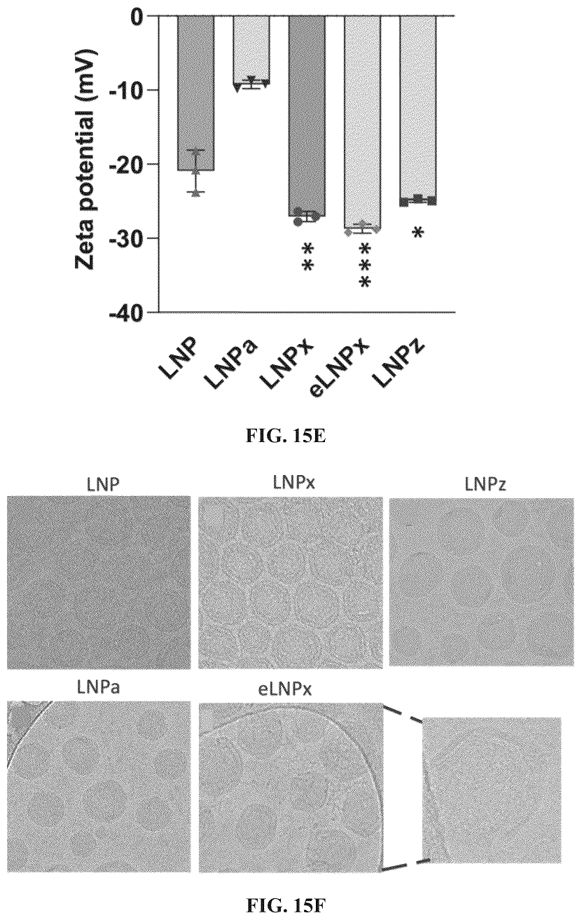

LNPs containing the ionizable lipid (DLin-MC3-DMA) or thiophene-based lipids (MG-lipids) described above can encapsulate the mRNA. LNPs have a hydrophobic core that is rich in ionizable lipids, sterols, and an oligonucleotide payload (FIG. 15A). Helper lipid (DSPC), lipid-PEG conjugate (DMG-PEG2k), and functional lipid-PEGs (DSPE-PEG2k-Amine/DSPE-PEG2k-Carboxy NHS/DSPE-PEG2k-Carboxylic acid) are abundant on the surface of LNP. Functional lipid PEGs referred to as DSPE-PEG2k lipids containing different reactive functional groups. The molar equivalent of the DMG-PEG2k component was replaced with various functional lipid-PEGs to formulate novel LNP variants (R-LNPs) (FIG. 15B). LNPs modified with various functional PEG containing lipids are referred to as R-LNPs. The DSPE-PEG2k-Amine-containing LNP system is referred to as an LNPa, the DSPE-PEG2k-Carboxy NHS-containing LNP system is referred to as an LNPx, the β-Sitosterol with DSPE-PEG2k-Carboxy NHS-containing LNP system is referred to as an eLNPx, and the DSPE-PEG2k-Carboxylic acid-containing LNP system is refer to as an LNPz. Aside from eLNPx, all LNP systems were prepared with cholesterol.

It was hypothesized that replacing some of the DMG-PEG2k with functional lipid-PEGs would change the surface potential of the current LNP system, allowing R-LNPs to be delivered differently in the neural retina. All the Cre mRNA encapsulated LNP systems had a narrow size distribution, with a hydrodynamic diameter of <90 nm, a PDI value of <0.21 and >94.8% encapsulation efficiency (FIGS. 15C, 15D). Using ZetaView, the surface potential of the LNP systems was measured (FIG. 15E). The zeta potential of a regular LNP was found to be around −20.9±2.8 mV. The lipid-PEG substitution in the current LNP system with functional DSPE-PEG2k-Amine reduces particle negativity to −9.3±0.6 mV. Highly anionic particles were observed in carboxylic PEG modified LNP variants. The changes in surface potential were compared DMG-PEG2k containing LNP system. The eLNPx system had a negative potential increase of −28.7±0.6 mV (1.4-fold, ***p<0.001), followed by the LNPx (−27.1±0.7 mV, 1.3-fold, **p<0.01) and LNPz systems (−24.9±0.2 mV, 1.2-fold, *p<0.05). Using cryo-transmission electron microscopy (cryo-TEM), the morphological changes of LNP systems were investigated. The Cholesterol-based LNP system showed spherical nanoparticles with electron-dense cores, whereas the β-Sitosterol modified eLNPx system showed a multi-faceted architecture (FIG. 15F). These findings support a previous study which found that replacing cholesterol with β-Sitosterol causes morphological changes. The results also demonstrated that replacing a portion of DMG-PEG2k with functional lipid-PEGs do not affect the morphology of R-LNPs.

### In-Vitro Performance of LNP Systems

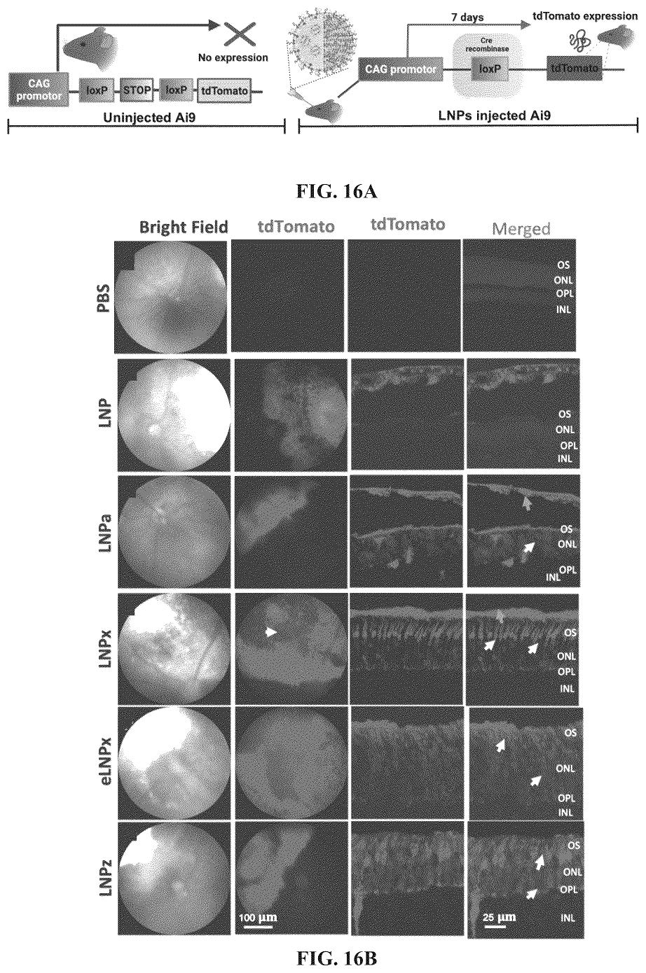

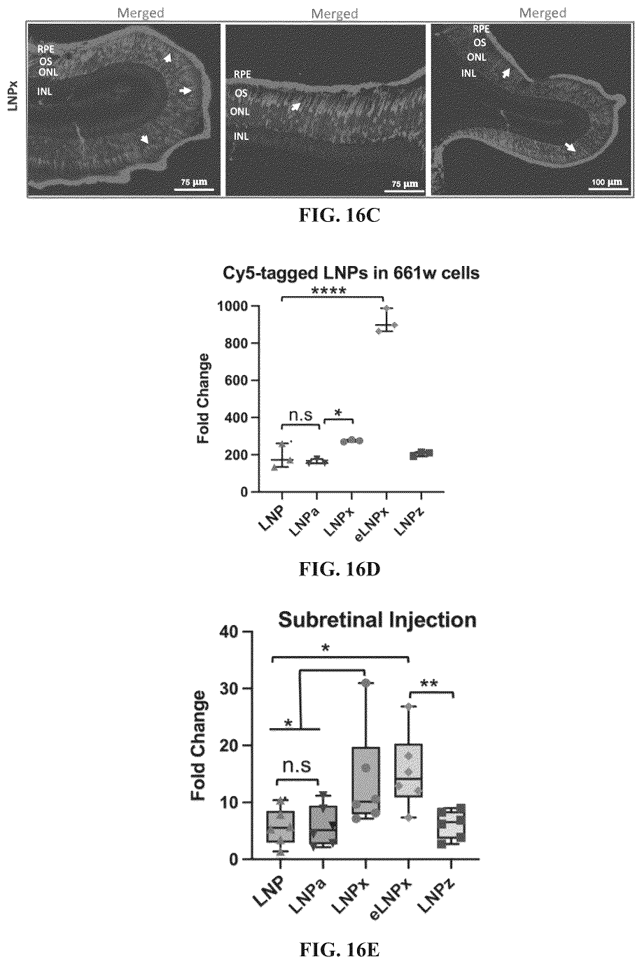

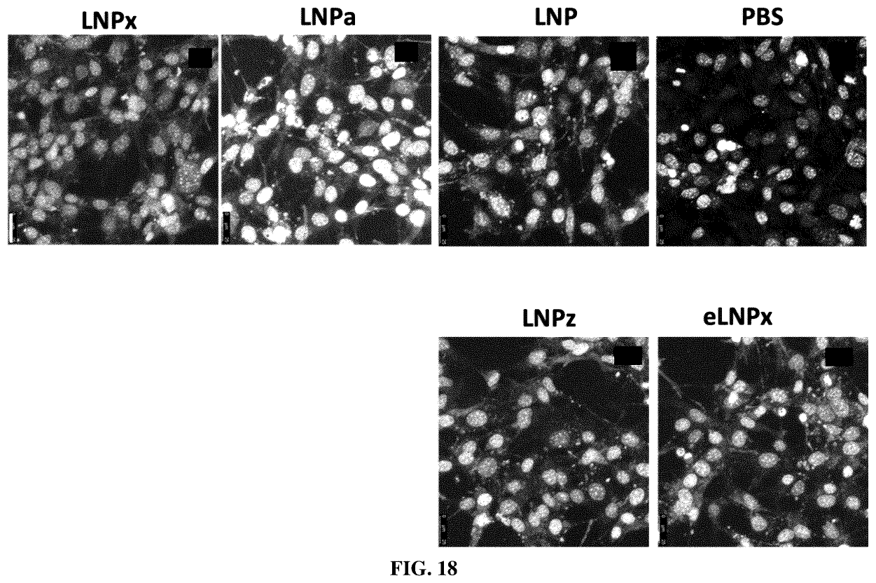

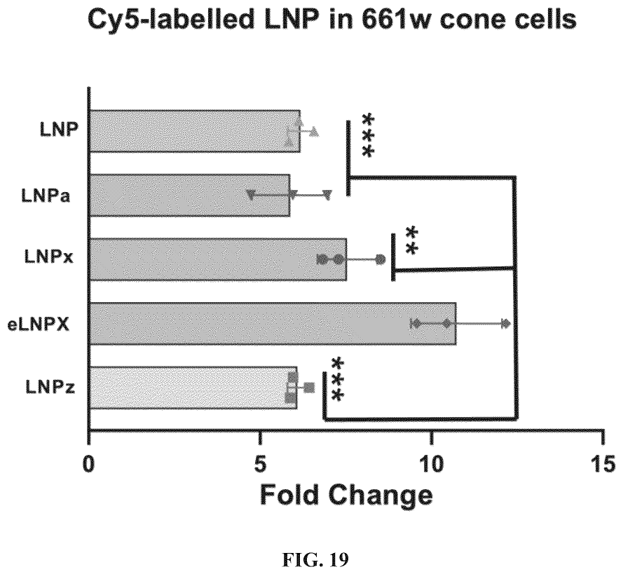

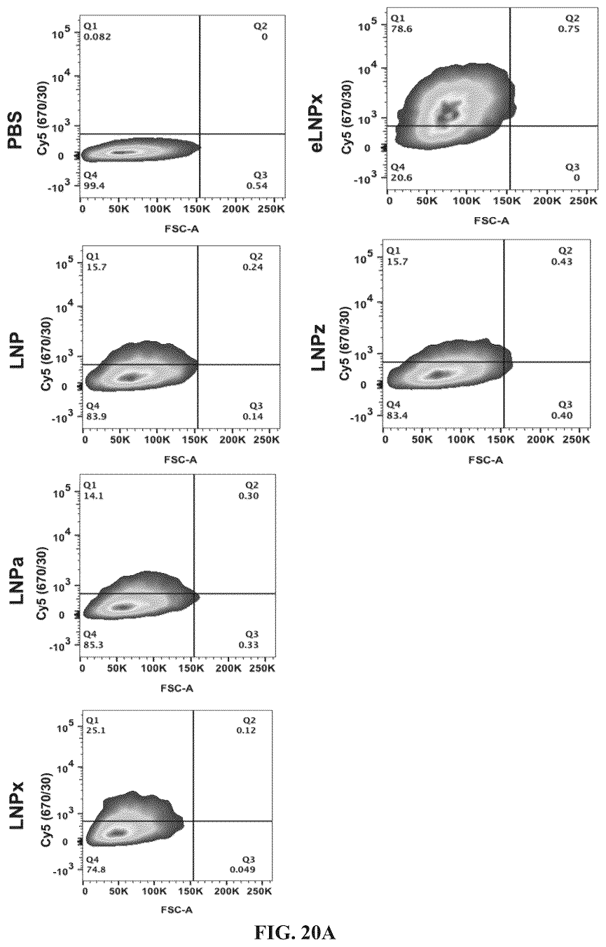

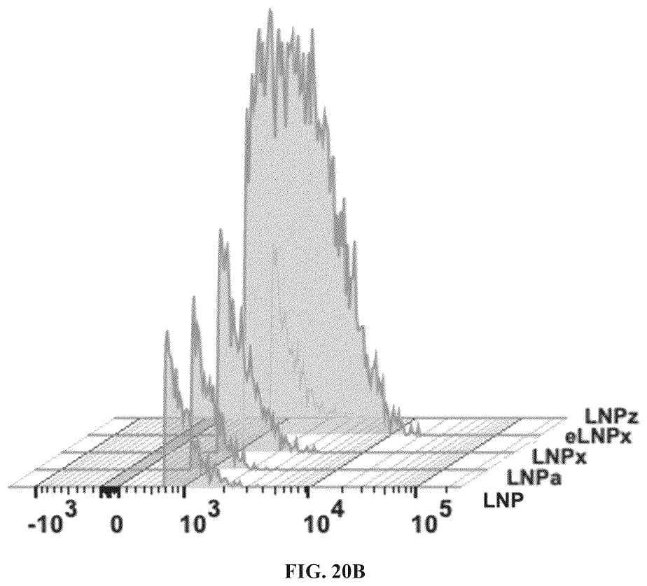

Intracellular trafficking is an arduous journey full of hurdles to beat for the genetic material. Cy-5 tagged mRNA was used as a cargo to confirm the intracellular uptake of LNP systems. The ocular cell line 661 w was selected as a model cell because changes in the sterol and functional lipid-PEGs could affect the cellular internalization of LNP systems. To confirm cellular trafficking, 661 w cone cells were treated with 100 ng of Cy-5 tagged LNP systems. Using confocal microscopy, the internalization of four novel LNP variants and regular LNP was compared to the PBS-treated groups (FIG. 18). Cells were fixed and stained for confocal imaging after a 24-hour incubation period. At 24 hours post-transfection, eLNPx uptake increased 10.7-fold (FIG. 19, **p 0.01, ***p 0.001), followed by LNPx uptake (7.5-fold). There were no significant differences in uptake between the LNP, LNPa, and LNPz treated groups. Flow cytometry analysis confirmed the cellular uptake of LNP-systems (FIGS. 20A to 20B). The results were consistent with the confocal microscopic observation that eLNPx internalization was significantly higher in cells (FIG. 16D, ****p 0.0001).

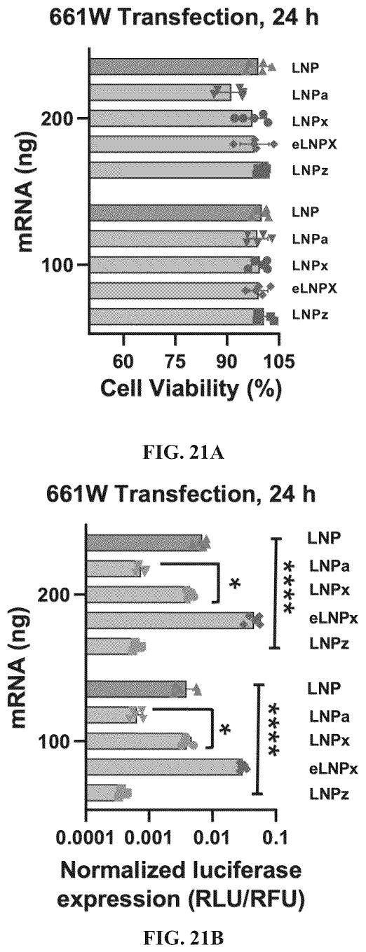

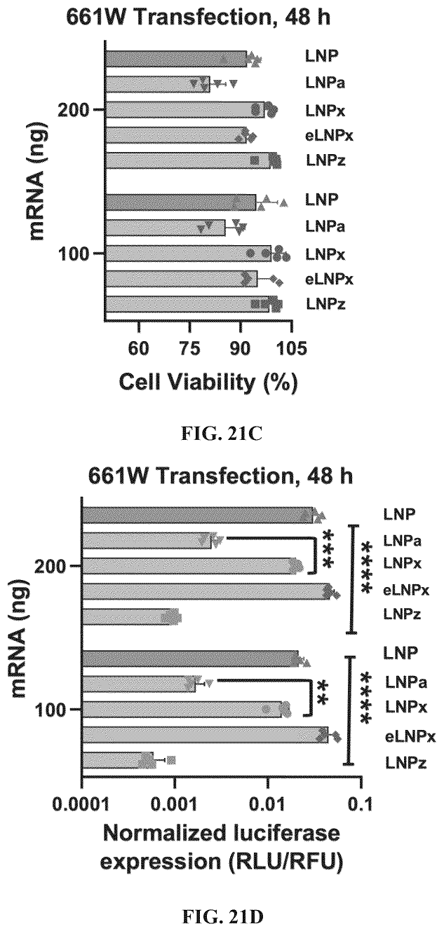

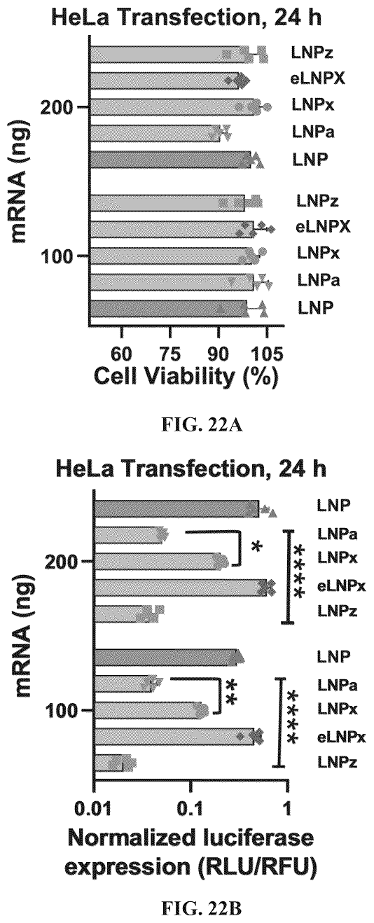

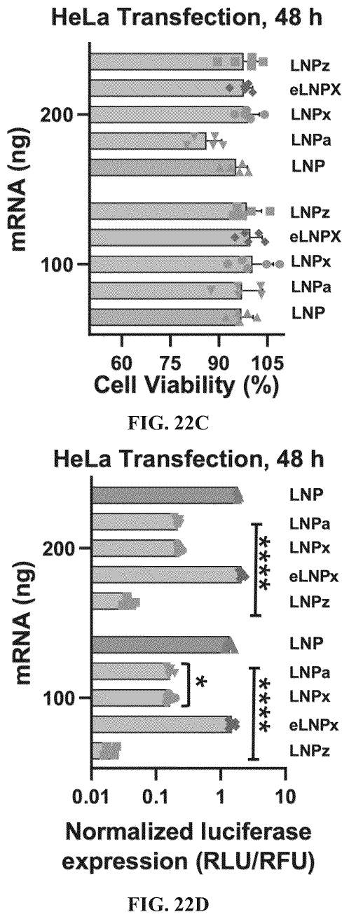

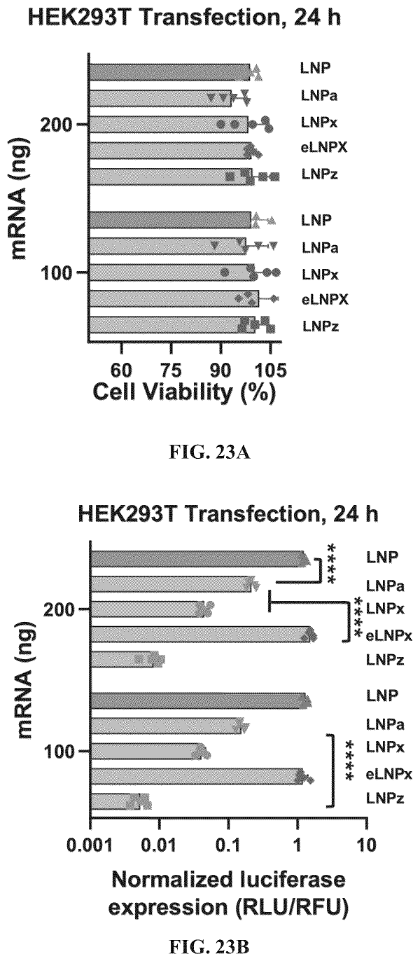

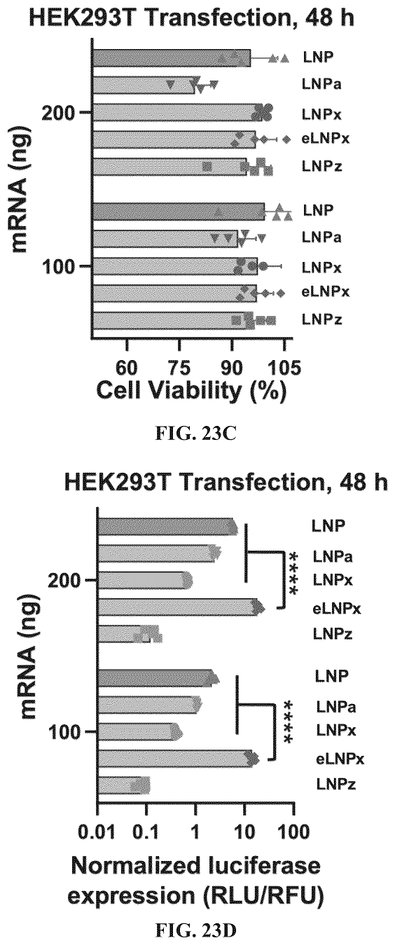

The Fluc mRNA transfection by LNP-systems in 661 w, HeLa, and HEK293T cells was then investigated at 24- and 48-hour time points (FIGS. 21A to 23D). At 100 and 200 nM concentrations, the cytotoxicity of LNP systems modified with different functional-lipid PEGs was compared. In three cell lines, the treatment of any LNP system did not compromise cell viability, except for the DSPE-PEG2k-Amine treated group. The LNP system-maintained cell viability (>91%) in 661 w cells for 24 hours, but LNPa treatment resulted in cell death (80.8%) after 48 hours of incubation (FIGS. 21A, 21C). At a higher mRNA concentration (200 nM), all LNP systems had similar effects on cell viability at 48 hours, but LNPa caused significant cell death in HeLa (86.1%, FIGS. 22A, 22C) and HEK293T (79.3%, FIGS. 23A,23C) cells at the same concentration, compared to other LNP variants. For 24 and 48 hours, time-dependent luciferase expression was observed in 661 w cells with LNP systems (FIGS. 21B, 21D). At the highest dose used, 200 ng per well, eLNPx, LNP, and LNPx produced 470.5-, 336.2-, and 213.3-fold higher luciferase expression at 48 hours than LNPa (26.6-fold) and LNPz (9.8-fold), implying their ability to induce endosomal escape. (**p 0.01, ***p 0.001, ****p 0.0001). Similarly, HeLa (FIG. 22B, 22D) and HEK293T (FIG. 23B, 23D) results showed that all LNP systems outperformed 661 w cells in terms of mRNA transfection in the 24- and 48-hour data collected. β-Sitosterol modified eLNPx demonstrated increased transfection efficiency in all cell lines, indicating that C-24 alkyl-substituted cholesterol analogs can improve transfection regardless of cell type.

PRs Receptor Targeting Efficiency of Cre mRNA Encapsulated LNP Variants

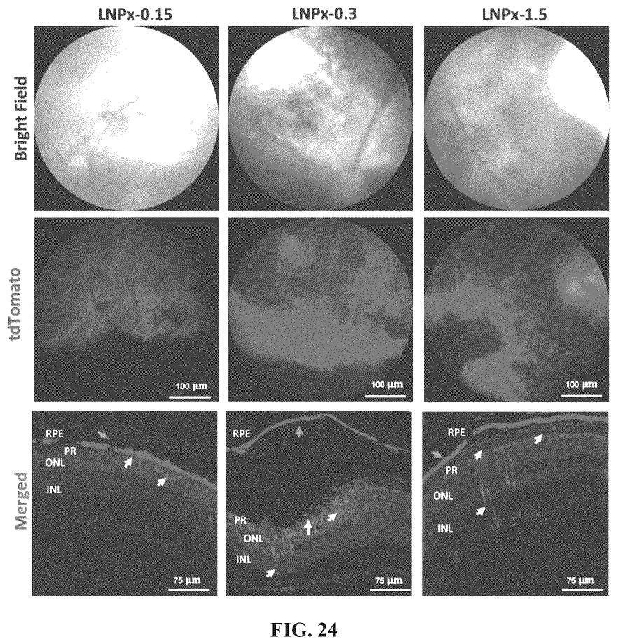

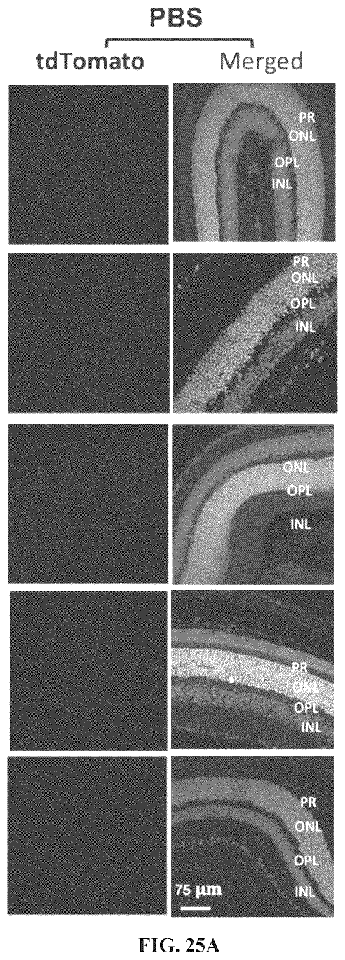

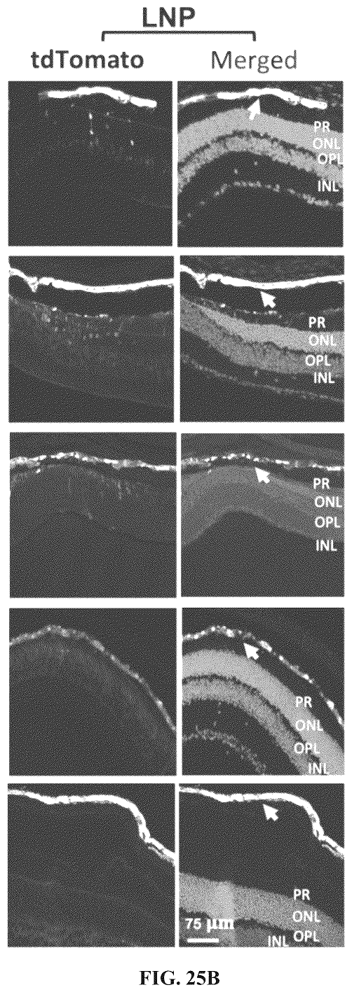

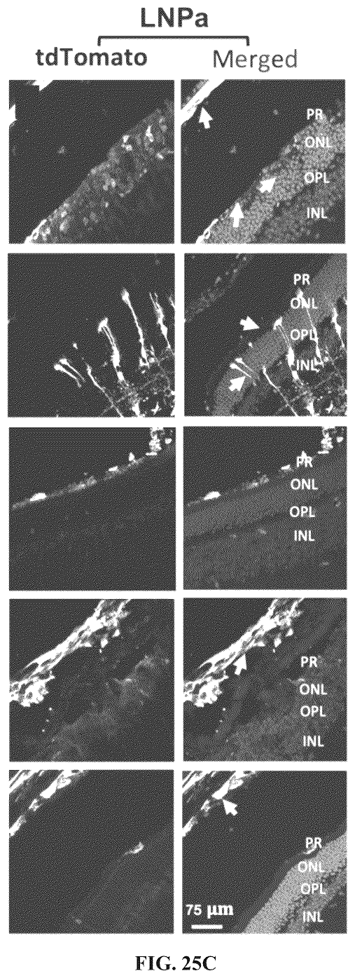

Most ocular disease therapeutics are currently focused on the treatment of the outer retinal cells (i.e., photoreceptors and RPE), and subretinal injections, which deliver the cargo directly to the target site, are being studied extensively. Cre-recombinase mRNA encapsulated LNP systems was delivered to test their ability to transduce PRs in-vivo (300 ng mRNA per eye). To formulate the novel LNPx variants, the percentage of DMG-PEG2k in regular LNP was reduced from 1.5 to 1.35 percent, 1.2 percent, or 0%. A wide range of DSPE-PEG-Carboxy NHS were tested, with 0.15% (LNPx-0.15), 0.3% (LNPx-0.3), and 1.5% (LNPx-1.5) of the total 1.5% DMG-PEG2k content, equivalent to 10-100% total PEG (Table 3). The final amount of PEG-lipids was not changed (i.e., 1.5%) because PEG-lipids affect the stability of the LNPs, and that range was maintained by replacing DMG-PEG2k-lipid with functional DSPE-PEG2k-Carboxy NHS. When the ratio of DMG-PEG2k to DSPE-PEG2k-Carboxy NHS was changed, the negative charge increased from −20.9±2.8 mV to −29.9±1.0 mV (1.4-fold, ***p 0.001, Table 4). LNP and LNPx variants were then subretinally injected into Ai9 mice, which stably express a floxed-stop codon upstream of a tdTomato cassette (FIG. 16A). tdTomato is only produced after the successful removal of the stop cassette by Cre recombination. Fundoscopy one week after injection revealed that tdTomato expression was exclusively increased in all LNPx variants treated groups (FIG. 24) compared to the PBS administered group (FIGS. 16B, 25A). Confocal microscopy was used to image the retina and assess neural retina distribution of the novel LNPx variants. Unexpectedly, it was observed that these LNPx variants targeted the PRs. In comparison to LNPx-0.15 and LNPx-1.5, a representative image of a retinal cross-section from an eye injected with the LNPx-0.3 variant revealed intense expression in PRs cells. These strong PRs targeting ability of novel LNPx-0.3 variants could be useful in the development of safe clinical applications in the future. tdTomato expression was restricted to the RPE in LNP-treated eyes (FIGS. 16C, 25B), and PBS-injected eyes were used to rule out retinal autofluorescence.

TABLE 3
Different lipid composition used to formulate LNPx particles.
The amount of DMG-PEG2000 was either partially
or completely replaced to check PRs targeting effect.
(MG-lipid = Thiophene based ionizable lipids)

| Composition | LNP | LNPx-0.15 | LNPx-0.3 | LNPx-1.5 |
|---|---|---|---|---|
| MC3 or MG lipid (%) | 50 | 50 | 50 | 50 |
| DSPC (%) | 10 | 10 | 10 | 10 |
| Cholesterol (%) | 38.5 | 38.5 | 38.5 | 38.5 |
| DMG-PEG2000 (%) | 1.5 | 1.35 | 1.2 | 0 |
| DSPE-PEG-2k-Carboxy | 0 | 0.15 | 0.3 | 1.5 |
| NHS (%) | | | | |

TABLE 4
Zeta potential measurement of regular
LNP and LNPx variants by Zeta View.
Surface charge (mV)

| | |
|---|---|
| LNP | −20.93 ± 2.8 |
| LNPx-0.15 | −27.07 ± 0.68 |
| LNPx-0.3 | −27.0 ± 0.73 |
| LNPx-1.5 | −29.97 ± 1.0 |

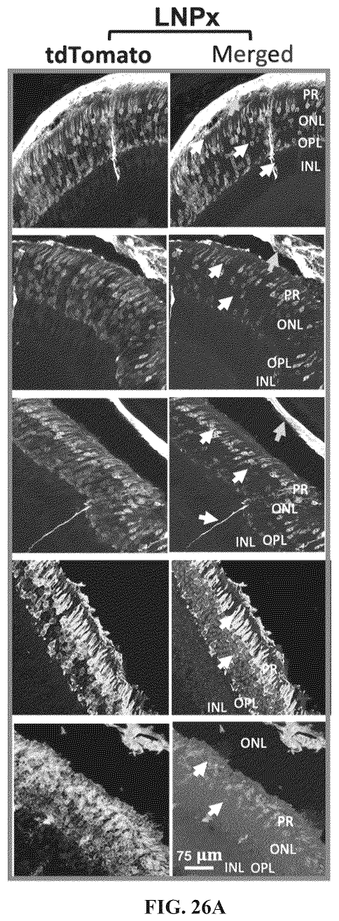

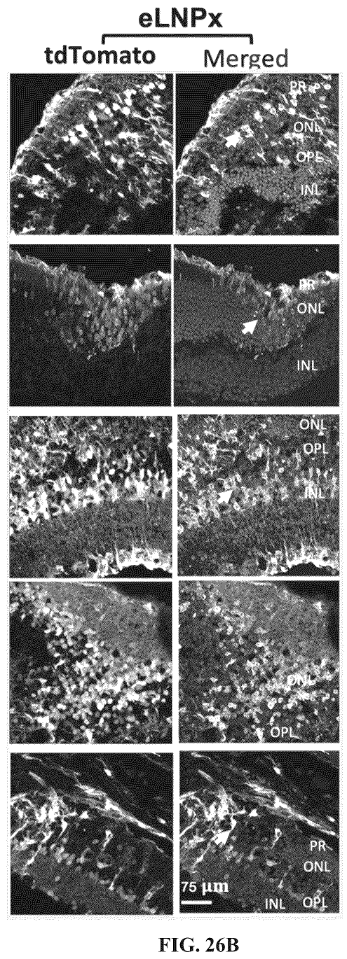

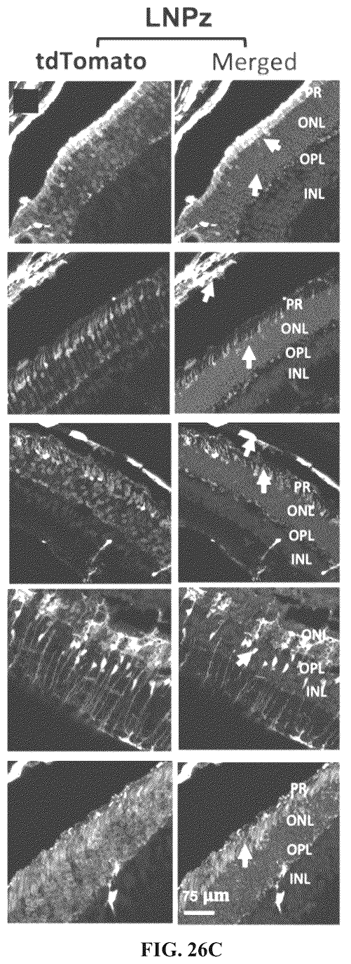

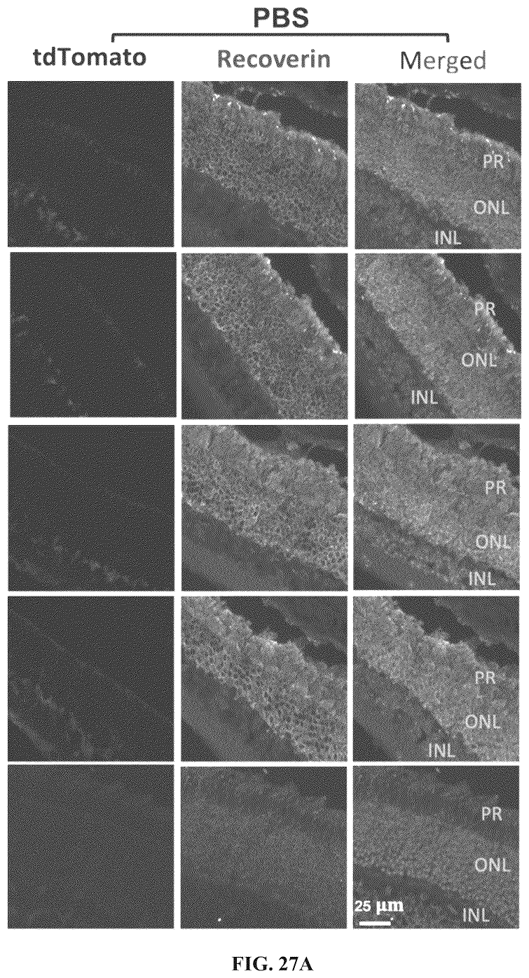

Following the initial in-vitro and in-vivo screening, it was decided to replace the regular LNP with various functional lipid-PEGs and β-Sitosterol. LNPx-0.3 variants were formulated using either cholesterol (LNPx) or β-Sitosterol (eLNPx) to evaluate the PRs targeting. Further to compare the targeting effect of the lipid-PEG carboxy-ester group, DSPE-PEG2k-Carboxy NHS of LNPx was replaced with DSPE-PEG2k-Carboxylic Acid for comparison. Regular LNP were also modified with DSPE-PEG2k-Amine (LNPa) and delivered via subretinal route in Ai9 mice to compare the effect with cationic functional lipid-PEGs. Fundus imaging revealed robust and selective tdTomato expression for all LNP variants (FIG. 16B). Fundus quantification data revealed that eLNPx had the highest tdTomato expression (15.4-fold), followed by LNPx (13.6-fold) via subretinal injection (FIG. 16E, *p0.05, **p0.01). The expression of LNP, LNPa, and LNPz was 5.7-, 5.8-, and 6.18-fold, respectively, with no significant differences between these groups. Confocal microscopy was used to examine the localization and expression of the mRNA in retinal cross-sections. Protein expression was mostly localized to the RPE in the LNPa-treated group with very limited expression in PRs and Müller glia (FIG. 25C). Retinal sections confirmed that LNPx and LNPz subretinal injections resulted in strong tdTomato expression in PRs and RPE (FIGS. 26A, 26C). With LNPx treatment, robust tdTomato expression was seen on an outer segment, an inner segment, the cell body, and the synaptic region of rod cells, and cone cells (low magnification images, FIG. 16C). When cholesterol was replaced with β-Sitosterol (eLNPx), expression was mostly seen in the RPE, ONL, and Müller glia (FIG. 26B), indicating efficient distribution in the retinal cells.

### Co-Localization of Photoreceptor Cells Using Specific Immunohistochemical Marker

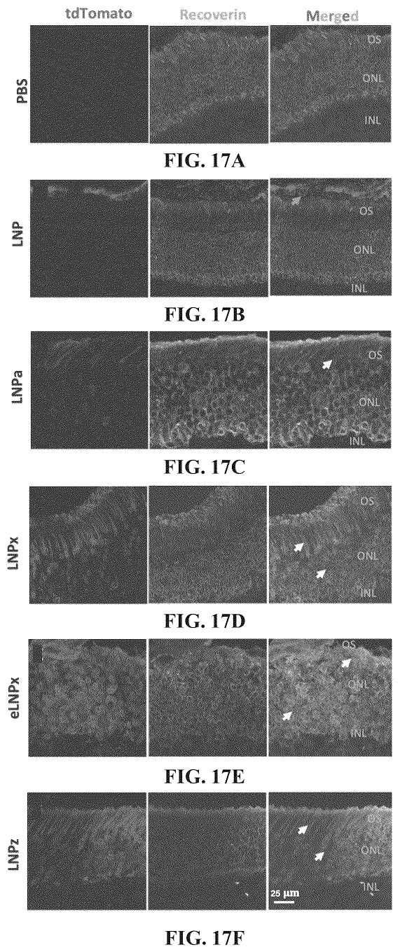

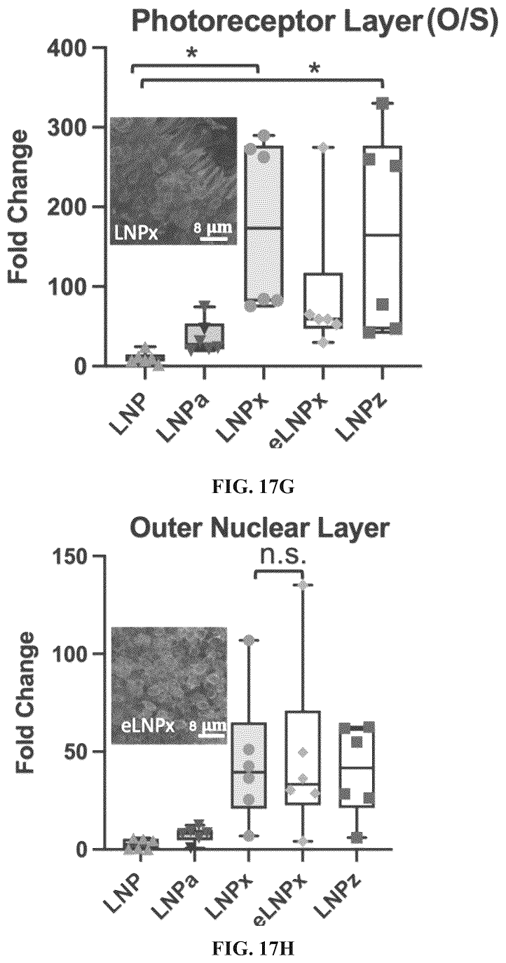

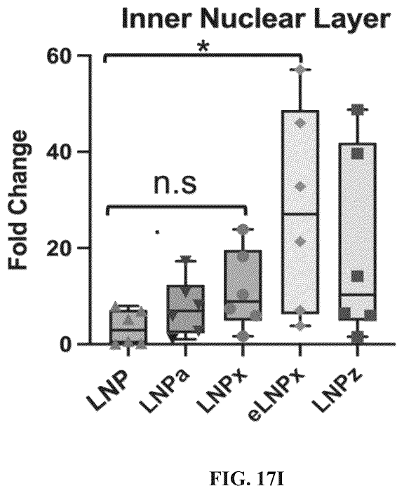

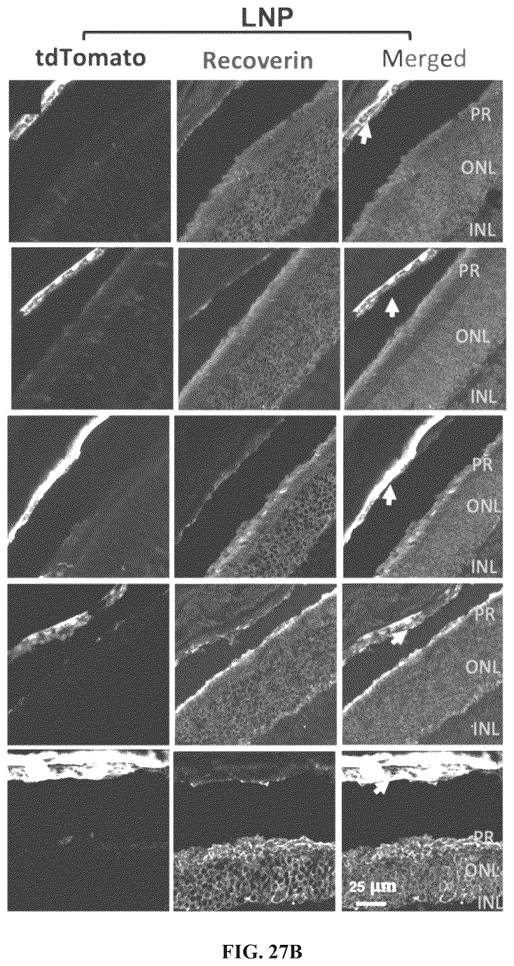

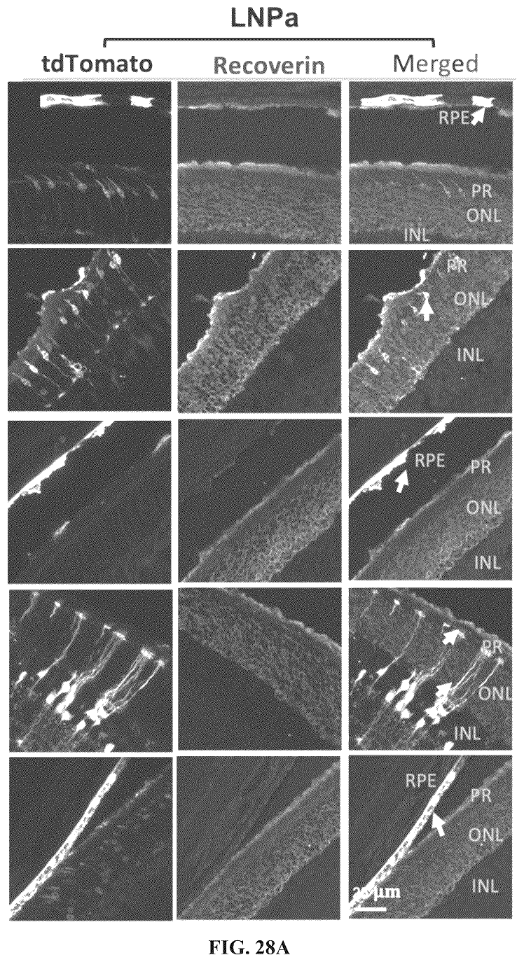

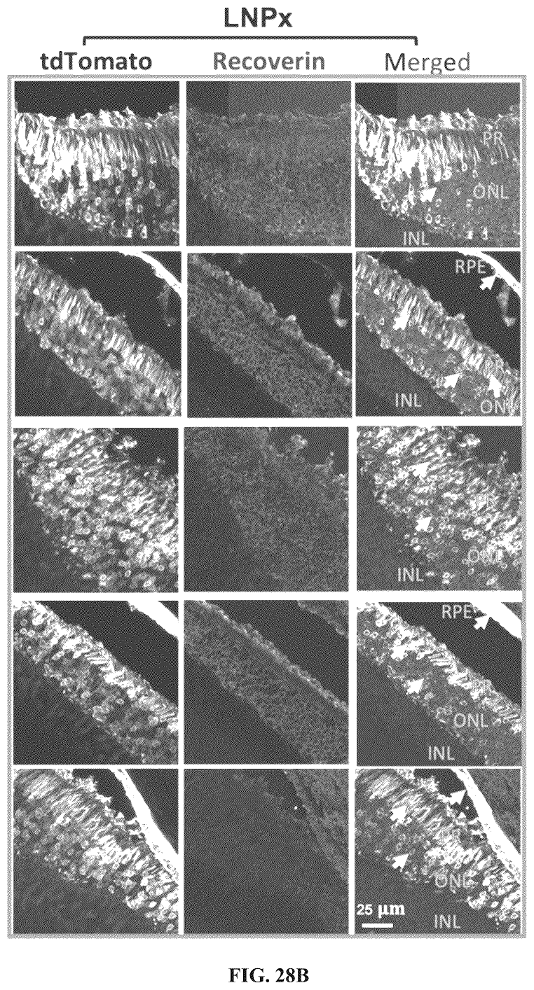

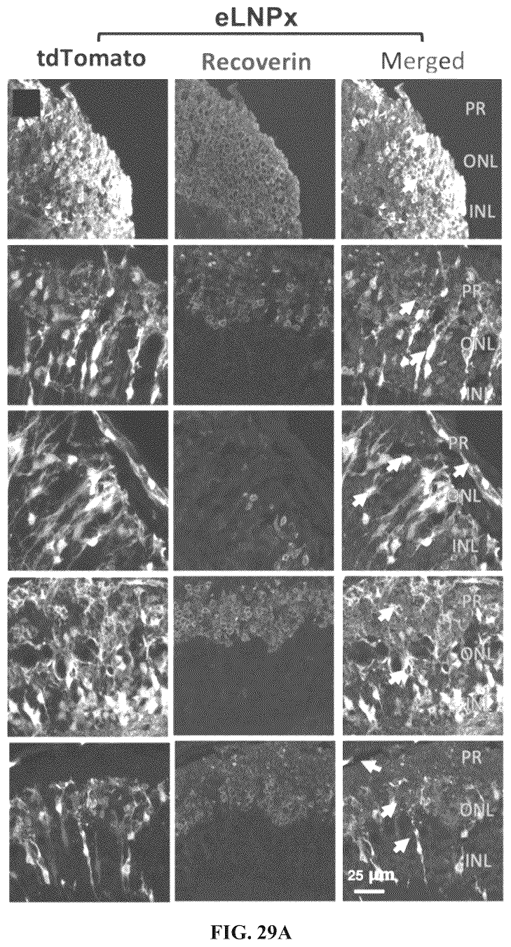

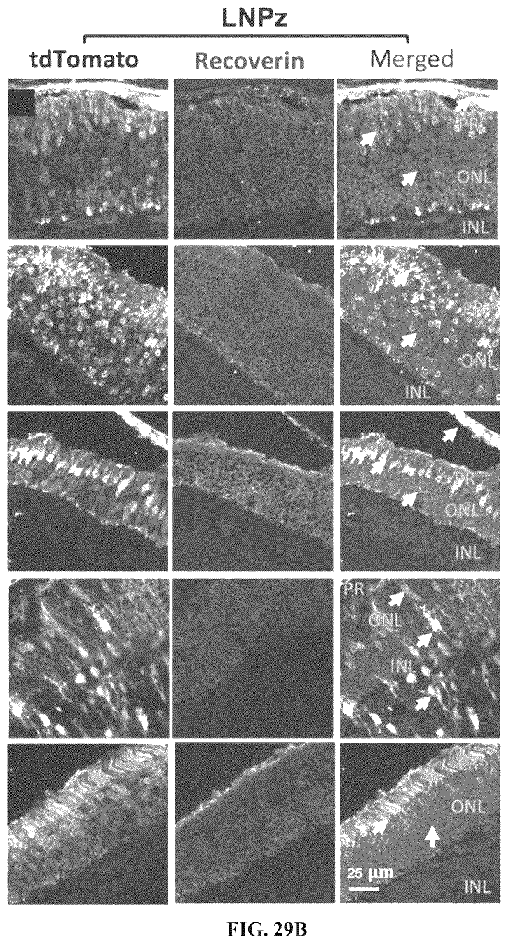

Immunohistochemistry (IHC) methods were used to show that tdTomato expression was co-localized with PRs cells using the recoverin antibody. Recoverin is a 23-kDa protein that is normally found in the retina's PRs cells and is involved in rhodopsin desensitization via Ca²⁺ -dependent regulation of rhodopsin kinase. Recoverin regulates visual transduction in rod and cone cells of the retina. IHC staining with recoverin revealed that most PRs cells were recoverin-positive (FIGS. 17A, 27A). The vast majority of tdTomato expression was restricted to RPE in LNP-treated retinas (FIGS. 17B, 27B). In the LNPa-treated groups, very few recoverin-stained PRs cells were co-localized (FIGS. 17C, 28A). In the LNPx (FIGS. 17D, 28B) and LNPz (FIGS. 17F, 29B) treated groups, tdTomato-positive cells were found in the RPE, PRs, outer nuclear layer (ONL), outer plexiform layer (OPL), and co-localized with recoverin stained PRs cells. Furthermore, most of the population of recoverin co-labeled td-Tomato expressing PRs cells were found in the ONL of eLNPx-treated groups (FIGS. 17E, 29A). Surprisingly, β-Sitosterol modification of LNPx resulted in high levels of tdTomato expression in Müller glia, implying that subretinal administration increased retinal penetration. In comparison to the PBS treated group, subretinal injection of LNPx resulted 177.8-fold increase in tdTomato (FIG. 17G, *p<0.05), followed by LNPz in the PRs outer segments (168.1-fold, *p<0.05). In the PRs layer, eLNPx, LNPa, and LNP showed 90.1-, 36.1-, and 7.19-fold tdTomato expression, respectively. The estimation of fluorescence intensity in ONL revealed that eLNPx, LNPx, and LNPz increased tdTomato expression by 47.4-, 44.9-, and 40.0-fold, respectively (FIG. 17H). Low expression was observed in both the LNPa (7.46-fold) and the LNP (2.8-fold) injected groups. The fluorescence intensity in the inner nuclear layer (INL) was measured to determine the retinal penetration ability of injected LNP systems. It was found that eLNPx treatment increased expression in the INL, particularly in Müller glia cells (27.9-fold, *p<0.05), followed by LNPz (19.4-fold) and LNPx (11.2-fold) injected groups. Overall, the DSPE-PEG-carboxy compound modification resulted in a highly anionic LNP system, and these LNPs specifically targeted the PRs cells. The findings also suggested that substituting β-Sitosterol for cholesterol may improve retinal penetration, which may be related to protein adsorption as the particle moves through the retina.

### Materials and Methods

Dlin-MC3-DMA can be purchased from BioFine International Inc, (BC, Canada). 1,2-distearoyl-sn-glycero-3-phosphocholine (18:0 PC, DSPC), 1,2-distearoyl-sn-glycero-3-phosphoethanolamine-N-[carboxy(polyethylene glycol)-2000, NHS ester] (DSPE-PEG2k-Carboxy NHS), 1,2-distearoyl-sn-glycero-3-phosphoethanolamine-N-[carboxy(polyethylene glycol)-2000](DSPE-PEG2k-Carboxylic acid), 1,2-distearoyl-sn-glycero-3-phosphoethanolamine-N-[amino(polyethylene glycol)-2000] (DSPE-PEG2k-Amine) was purchased from Avanti Polar Lipids. Cholesterol, β-Sitosterol and 1,2-dimyristoyl-rac-glycero-3-methoxypolyethylene glycol-(DMG-PEG2k) was purchased from Sigma Aldrich. To encapsulate cargo into LNPs, Cre mRNA (5moU)—(L-7211) and Fluc mRNA—(L-7602) were purchased from Trilink Biotechnologies. Anti-Recoverin was purchased from Millipore Corp. USA. MG-lipids (Thiophene based ionizable lipids) when used in formulation were designed at Oregon State University as described above.

### Cell Culture

All cell culture media and supplies were obtained from Thermo Fisher Scientific (Waltham, MA). 661 W cone cells were generously provided by Prof. Muayyad Al-Ubaidi, University of Houston, Houston, TX. 661 w cells were cultured in Dulbecco's modified Eagle's medium (DMEM) supplemented with 10% heat-inactivated fetal bovine serum (FBS) (Hyclone Laboratories Inc., Logan, UT), 1×penicillin/streptomycin (Thermo Fisher, Federal Way, WA), 23 mg/l Putrescine, 40 μl of β-Mercaptoethanol, 300 mg/l glutamine, and 40 μg of hydrocortisone 21-hemisuccinate and progesterone. Human Embryonic Kidney 293T/17 cells (CRL-11268; ATCC, Manassas, VA) and HeLa cells were also cultured in DMEM supplemented with 10% FBS and 1% penicillin/streptomycin. All the cells were maintained in an atmosphere of 5% CO₂ at 37° C.

### Animals

Ai9(RCL-tdT), C57BL6 mice were purchased from The Jackson Laboratory (Bar Harbor, ME, USA). Ai9 is a Cre reporter tool designed to have a loxP-flanked STOP cassette preventing transcription of tdTomato under the control of a ubiquitous promoter. Following Cre-mediated recombination Ai9, mice express robust tdTomato. All the mice used in the experiments were about 1-6 months old and were either bred in-house or used directly for the studies.

Formulation of mRNA LNPs and Characterization

To obtain the desired molar ratio in the organic phase, mRNA encapsulated LNPs were prepared using a total lipid concentration of 5.5 mM comprised of ionizable lipid (DLin-MC3-DMA or MG-lipids(thiophene based lipids))/DSPC/Sterols (Cholesterol or β-Sitosterol)/DMG-PEG2k and/or functional PEGs at the mol percent ratio of 50/10/38.5/1.2/0.3 in the ethanol solution. Before microfluidic mixing in the NanoAssmblr™ (Precision NanoSystem) at a 3:1 flow ratio, mRNA was dissolved in 50 mM citrate buffer pH 4.0, keeping the N/P (ionizable lipid: mRNA) ratio constant at 5.67. Following formulation, LNPs were diluted in sterile DPBS (pH 7.4) and dialyzed against 3 lit of PBS for 4 hours at room temperature using a 10 KDa Slide-A-Lyzer dialysis bag, before being transferred to fresh PBS solution overnight. LNPs were concentrated after dialysis using a MWCO, 10 KDa Amicon Ultra-4 mL centrifugal filter unit for in-vitro and in-vivo studies (Millipore, Burlington, MA). LNP variants' size distribution and PDI were determined using Dynamic Light Scattering (DLS) in the Zetasizer Nano ZS (Malvern Panalytical Inc., Westborough MA) with a 1×PBS solution at 25° C. Using a ZetaView TWIN equipped with a video microscope PMX-420 (Germany), the net LNPs surface charge was determined using Nanoparticle Tracking Analysis (NTA). Quant-iT RiboGreen RNA reagent was used to estimate mRNA encapsulation efficiency and concentration.

### Cryo-TEM Image Acquisition and Processing

Falcon III and K3 Summit cameras with DED at 300 kV were used to capture cryo-TEM images. The Vitrobot Mark IV system (FEI) was used to plunge-freeze a copper lacey carbon film-coated Cryo-EM grid (Quantifoil, R1.2/1.3 300 Cu mesh). 2 μL of LNP was dispensed onto the glow discharged grids in the Vitrobot chamber maintained at a temperature of 23° C. and a relative humidity of 100% to freeze the samples. The sample was incubated for 30 seconds before being blotted with filter paper for 3 seconds before being submerged in liquid ethane cooled by liquid nitrogen. The frozen grids were meticulously examined for any defects, clipped, and assembled into cassettes. The images were taken at an electron dose of 15-20 e−/Å2 using 45,000 nominal magnifications with 1.5 binning then processed and analyzed using Fiji.

### Cellular Uptake of Cy-5 Tagged Lipid Nanoparticles

Cy5-tagged LNPs and R-LNPs were used to study the cellular uptake in 661 w cone cells. 661 w cells were seeded at a density of 60,000 per well in an 8-well μ-Slide (Ibidi, Fitchburg, WI) for confocal microscopy. All cells were treated with Cy5-tagged LNPs and R-LNPs in a dose equivalent to 100 ng/well of mRNA cargo, incubated for 24 hours at 37° C., washed with 1×DPBS, and fixed for 7 minutes at room temperature with 4% paraformaldehyde. Cells were then washed twice with 1×DPBS, stained with DAPI (0.75×), washed again, and mounted with Fluoromount-G Mounting Medium (Thermo Fisher cat. #00-4958-02) for confocal microscopy imaging. The fluorescence intensity of images was analyzed using the National Institutes of Health ImageJ software after the same exposure parameters were set for the PBS and LNPs treated groups (version 1.45; Bethesda, MD).

Flow cytometry was used to confirm cellular uptake of the particles. Approximately 160,000 661 w cells were seeded per well in 6 well plates and kept at 37° C. for 24 hours in a 5% CO₂ atmosphere. Cells were treated with 100 ng/well equivalents of mRNA from Cy5-tagged LNPs and incubated for 24 hours. Following incubation, cells were washed twice with flow cytometry staining buffer (eBioscience, cat. #00-4222-26), tyrpsinized using Trypsin (0.25%)-phenol red (Gibco, cat. #15050065), harvested and re-suspended in the FACS buffer then flow cytometry analyzed (BD FACS Diva v8.0.1 software). A 670/30 bandpass filter was used to detect the Cy-5 signal.

### In-Vitro Transfection

A cell density of 4000/well was plated in white, clear-bottom 96-well plates and allowed to grow to 60-70% confluency for 24 hours at 37° C. 661 w, HeLa, and HEK293T cells were treated with LNPs and R-LNPs encapsulating Fluc mRNA at concentrations of 100 and 200 nM in the medium in all transfection studies. All the cells were incubated for an additional 24 and 48 hours. Cell viability (Promega CellTiter Fluor™ Cell Viability Assay) and luciferase expression (Promega One-Glo™ Luciferase Assay) were assessed. Cell viability was used to normalize luciferase expression.

### Subretinal Injection

Before the subretinal injection, the eyes were dilated by topical administration of 0.5% proparacaine, 1% tropicamide, and 2.5% phenylephrine and anesthetized with ketamine (100 mg/kg)/xylazine (10 mg/kg) cocktail administered intraperitoneally. To initiate the injection, 2.5% hypromellose was placed over the eye and a 30-gauge needle was used to make an incision in the limbus. After that, a glass coverslip was placed over the eye to allow visualization of the retina. Using a Hamilton syringe with a 33-gauge blunt needle, 1 μL of PBS or Cre-LNPs/R-LNPs (300 ng mRNA/injection) were delivered to the subretinal space through the scleral incision in the limbus. To test for retinal detachment, a 2% fluorescein solution was added to the PBS, LNPs, and R-LNPs. Scleral incisions in the limbus were made for most injections, and PBS, LNPs, and R-LNPs were delivered temporally.

### Fundoscopy

Fundoscopy of Cre-LNPs/R-LNPs injected in-vivo retina imaging was performed 1 week after administration using Micron IV (Phoenix Research Laboratories, Pleasanton, CA). To visualize general retinal health, tdTomato expression, bright field, and red-fluorescent images were taken. All the experiments used the same exposure settings.

### Tissue Preparation

Cre-LNPs/R-LNPs injected mice were sacrificed immediately after fundus imaging. The limbus of PBS and formulation injected eyes were marked at the 12 o'clock position with a hot needle to aid orientation. Under dim light, eyes were enucleated and immediately placed in 4% paraformaldehyde overnight at 4° C. Eyes were prepared for cryopreservation using the methods described previously¹⁵. In brief, corneas were removed from each eye, leaving the lens inside the remaining eyecup. To maintain orientation, a small “V” shaped cut was made into the sclera adjacent to the burned limbus. After fixation, the lens and vitreous were removed then the retina/RPE-containing eyecup was incubated in 30% sucrose in PBS for 2 hours at 4° C. before embedding in cryostat compound (Tissue-Tek O.C.T. Compound, code #4583). The embedded eyes were snap-frozen in a dry ice bath. Eyes were sectioned at 12 microns with a cryostat (Microtome HM550; Walldorf, Germany) and kept at −20° C.

### Immunohistochemistry (IHC) and Confocal Microscopy

After removing retinal cryosections from the freezer, they were dried for at least 30 minutes and washed three times in 1×PBS. After incubating samples in 0.3% Triton X-100 solution for 1 hour at room temperature in the dark, they were blocked in a Super blocking buffer (Thermo Scientific, Cat. #37515) for 1 hour. Primary antibody diluted in 0.1 percent Triton X-100 in Super blocking buffer was incubated overnight at 4° C. on retinal sections. Following primary antibody incubation, retinal sections were washed three times with 1×PBS before being incubated with 0.1% Triton X-100 diluted secondary antibody for one hour at room temperature and washed three times with 1×PBS. All the retinal sections were counter-stained with DAPI for 5 min at room temperature. After a final rinse, retinal sections were mounted in a Fluoromount-G, and cover-slipped. For Ai9 mice cryosections, anti-recoverin rabbit antibody (1:500, Sigma-Aldrich, Cat. #AB5585) and corresponding goat anti-rabbit Alexa Fluor 700-IgG (H+L) (1:1000, Invitrogen Cat. #A21038) were used for detection. Retinal sections were analyzed by confocal microscope equipped with TCS SP8 X (Leica Microsystems, Buffalo Grove, IL). All the images were captured with identical exposure setting at either 40× or 60× magnification using Z-stacks. Maximum fluorescence intensity was calculated in different retinal layers and the result was plotted in GraphPad Prism (version 9.3.1) for graphical presentation.

### Statistical Analysis

For fundus quantification, the region of interest (ROI) was created for the whole eye to calculate the mean fluorescence intensity using ImageJ. Also, the PR layer, ONL layer, and INL layer were outlined in the retinal section of Ai9 mice to measure the tdTomato expression. Fold change values were calculated by comparing to PBS injected group. An ordinary one-way ANOVA analysis, with Tukey's multiple comparison test, was used between groups using GraphPad Prism. All the data were presented as mean±SEM. A p-value<0.05 was considered statistically significant.

### Example 16

In this example, R¹, R², and R³ correspond to aliphatic of R^{e}, aliphatic of R⁴, and aliphatic of R⁷, respectively, in Formula II.

The condensed synthesis strategy is based on a modular approach using the Gewald Reaction to enable a facile access to the general structure shown below. In certain embodiments, R¹ and R² are comprised of aliphatic moieties, and R³ is comprised of an ionizable moiety (e.g., a basic nitrogen atom).

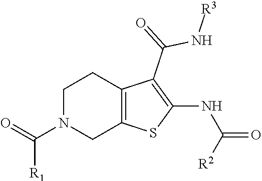

The Gewald Reaction enables the synthesis of an advanced intermediate (Procedure C). A subsequent addition of an acid chloride finishes the total synthesis of the depicted thiophene based lipids.

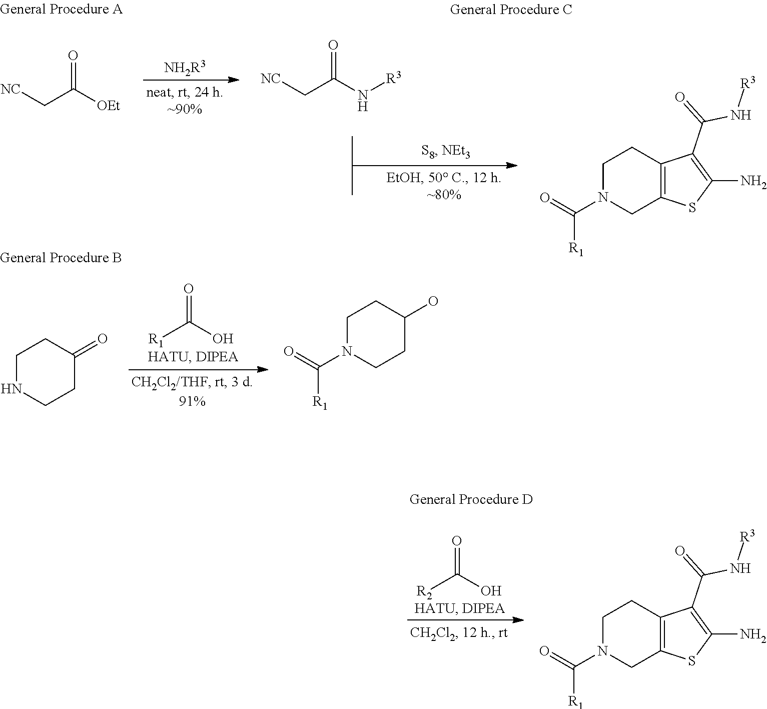

General Procedure A: 2-Isocyanoacetamides: A Schlenk flask was charged with an amine (1.0 equiv.) and ethyl 2-isocyanoacetate (1.0 equiv.) under N₂-atmosphere. The reaction mixture was stirred for 48 hours. For primary amines the reaction proceeded at room temperature. Reaction mixtures containing secondary amines were additionally heated using an oil bath to ensure conversion of the starting material. The reaction was monitored by TLC. After complete conversion of starting material, the desired 2-Isocyanoacetamides were isolated by vacuum distillation or column chromatography.

General Procedure B: Modified piperidin-4-one: A Schlenk was charged with a solution of the desired acid (1.1 equiv.) in anhydrous THF:CH₂Cl₂ (1:1) under N₂-atmosphere. DIPEA (2.5 equiv.) and HATU or HBTU (2.2 equiv.) were added successively and the reaction mixture was stirred for 15 minutes before the addition of 4-piperidone monohydrate hydrochloride (1.0 equiv.). The reaction mixture was left to stir for 72 hours at room temperature. The reaction was quenched with saturated NaHCO₃ (aq) and extracted with CH₂Cl₂ (3×). The combined organic layers were washed with brine, dried over MgSO₄, filtered, and concentrated under reduced pressure. The residue was purified by column chromatography.

General Procedure C: Gewald Reaction: A Schlenk flask was charged with the desired 2-isocyanoacetamide (1.0 equiv.), a modified piperidin-4-one (1 equiv.), sulfur (1.0 equiv.), EtOH and NEt₃ (1.0 equiv.) under N₂-atmosphere. The reaction mixture was stirred overnight at 50° C. After allowing the reaction mixture to cool down to room temperature the solvent was removed under reduced pressure and the crude residue was purified by column chromatography. To remove the NEt₃-HCl salt the isolated mixture was dissolved in anhydrous THE and solids were filtered off, and the solvent removed under reduced pressure to yield the desired thiophene.

General Procedure D: A Schlenk flask was charged with acid (1.0 equiv.) and DMF (0.1 equiv.) in anhydrous CH₂Cl₂ under N₂-atmosphere. Oxalyl chloride (1.5 equiv.) was added and the reaction mixture stirred for 3 hours at room temperature. The solvent was removed under reduced pressure and the crude material was dissolved in CH₂Cl₂. After addition of thiophene (1.0 equiv.) the reaction mixture was stirred overnight at room temperature. The solvent was removed under reduced pressure and the crude residue purified by column chromatography. To remove the NEt₃-HCl salt the isolated mixture was dissolved in anhydrous THE and solids were filtered off, and the solvent removed under reduced pressure to yield the desired thiophene lipid.

### EQUIVALENTS AND SCOPE

In the claims and throughout, articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following claims.

We claim:

1. A compound according to Formula A 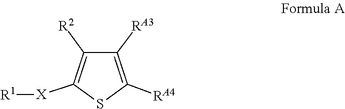
wherein:
X is a bond, —C(O)O—, —OC(O)—, —NH—, —N(R¹)—, —C(O)N(R¹)—, —N(R¹)C(O)—, —C(O)NH—, —NHC(O)—, —C≡C—, —CH═CH—, —O—, —S—, —NHC(O)NH—, —OC(O)O—, —OC(O)NH—, —NHC(O)O—, —O(CH₂)ₘ or —(CH₂)ₘO—;
m is from 0 to 6;
each R¹ is H, halogen, CN, or aliphatic;
R² is H, halogen, aromatic, or aliphatic;
or X is a bond and R¹ and R², together with the atoms to which there are attached, form a 4- to 7-membered nitrogen-containing non-aromatic heterocyclyl;
R^{A3} is halogen, R³, —C(O)N(R⁶)(R⁷), —C(O)R³, —CN, —(CH₂)ᵣN(R⁶)(R⁷), —N(R⁶)(R⁷), —S(O)R^{d}, —S(O)R³, —CH₂SR^{d}, —CH₂R³, —S(O)N(R⁶)(R⁷), or —X¹(CH₂)ᵣN(R^{a})₂;
X¹ is O, S or NH;
r is from 1 to 8;
each R^{a} independently is H, C₁₋₆alkyl, C₃₋₆cycloalkyl, or both R^{a}s together with the nitrogen to which they are attached form a 5- or 6-membered heterocyclyl;
R³ is aliphatic, —O-aliphatic or N(R⁶)(R⁷);
R^{A4} is halogen, boronic acid, —N(R⁴)(R⁵), NHC(O)N(R⁴)(R⁵), —N═CHR^{d}, —NHC(S)N(R⁴)(R⁵), —NHC(O)(CH₂)_{z}—N(R⁴)(R⁵), —NHSO₂R^{d}, —NHS(O)R^{d}, —NHC(O)C(O)NH(CH₂)_{z}—N(R⁴)(R⁵), or —OR⁴, where z is an integer from 1 to 6;
each R^{d} is aliphatic;
R⁴ is aliphatic or —C(O)aliphatic;
R⁵ is H, aliphatic, or —C(O)aliphatic;
R⁶ is H or aliphatic;
R⁷ is aliphatic or C(O)aliphatic;
or R⁶ and R⁷, together with the atom to which there are attached, form a 4- to 7-membered nitrogen-containing heterocyclyl; and
wherein the compound comprises at least one C₆₋₃₀aliphatic moiety.

2. The compound of claim 1, wherein:
R⁶ is H or aliphatic; and
R⁷ is aliphatic or C(O)aliphatic.

3. The compound of claim 1, having a Formula I: 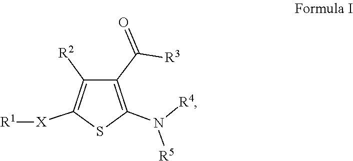
wherein X is a bond, —NH—, —N(R¹)—, or —OC(O)—.

4. The compound of claim 1, having a Formula I-A:

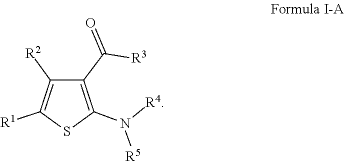

5. The compound of claim 4, wherein
R¹ is aliphatic;
R² is H or aliphatic;
or R¹ and R², together with the atoms to which there are attached, form a 5- to 7-membered nitrogen-containing non-aromatic heterocyclyl;
R³ is aliphatic, —O-aliphatic or N(R⁶)(R⁷);
R⁴ is aliphatic or —C(O)aliphatic;
R⁵ is H, aliphatic or —C(O)aliphatic;
R⁶ is H or aliphatic;
R⁷ is aliphatic or C(O)aliphatic; and
wherein the compound comprises at least one C₆₋₃₀aliphatic moiety.

6. The compound of claim 1, wherein R¹ is C₆₋₃₀aliphatic.

7. The compound of claim 1, wherein R¹ is C₆₋₁₀aliphatic.

8. The compound of claim 1, wherein R¹ is C₁₋₆alkyl and is substituted with N(R^{a})₂, where each R^{a} independently is H or C₁₋₆alkyl, or both R^{a}s together with the nitrogen to which they are attached form an optionally substituted 5- or 6-membered heterocyclyl.

9. The compound of claim 8, wherein R¹ is —(CH₂)₁₄N(R^{a})₂.

10. The compound of claim 9, wherein R¹ is —CH₂NH₂.

11. The compound of claim 1, wherein R² is H.

12. The compound of claim 1, wherein R² is C₆₋₃₀aliphatic.

13. The compound of claim 1, wherein R¹ and R² are both C₆₋₃₀aliphatic, and R¹ and R² are different.

14. The compound of claim 1, wherein the compound has a formula 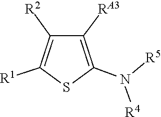
wherein each of R¹ and R² independently is C₆₋₃₀aliphatic.

15. The compound of claim 1, wherein the compound has a formula 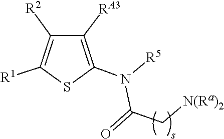
wherein:
each of R¹ and R² independently is C₆₋₃₀aliphatic;
each R^{a} independently is H or C₁₋₆alkyl, or both R^{a}s together with the nitrogen to which they are attached form an optionally substituted 5- or 6-membered heterocyclyl; and
s is an integer from 1 to 8.

16. The compound of claim 1, wherein the compound has a formula 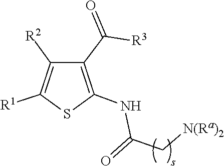
wherein:
each of R¹ and R² independently is C₆₋₃₀aliphatic;
each R^{a} independently is H or C₁₋₆alkyl, or both R^{a}s together with the nitrogen to which they are attached form an optionally substituted 5- or 6-membered heterocyclyl; and
s is an integer from 1 to 8.

17. The compound of claim 1, wherein the compound has a formula 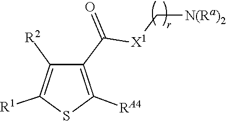
wherein:
each of R¹ and R² independently is C₆₋₃₀aliphatic; and
each R^{a} independently is H or C₁₋₆alkyl, or both R^{a}s together with the nitrogen to which they are attached form an optionally substituted 5- or 6-membered heterocyclyl.

18. The compound of claim 17, wherein the compound has a formula

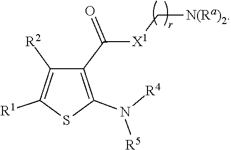

19. The compound of claim 1, wherein the compound has a formula 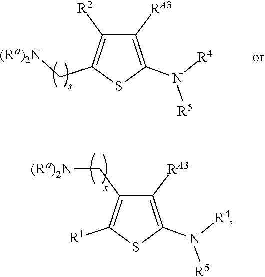
wherein:
each R^{a} independently is H or C₁₋₆alkyl, or both R^{a}s together with the nitrogen to which they are attached form an optionally substituted 5- or 6-membered heterocyclyl; and
s is an integer from 1 to 8.

20. The compound of claim 19, wherein the compound has a formula

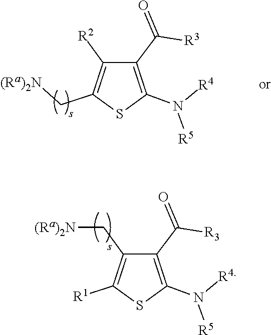

21. The compound of claim 15, wherein s is an integer from 1, 2, 3 or 4.

22. The compound of claim 8, wherein each R^{a} independently is C₁₋₆alkyl.

23. The compound of claim 16, wherein:
s is 2 or 3;
each of R¹ and R² independently is C₆₋₁₀alkyl;
R³ is O-methyl or O-ethyl;
each R^{a} independently is C₁₄alkyl; or
a combination thereof.

24. The compound of claim 1, wherein X is a bond and R¹ and R², together with the atoms to which there are attached, form a 4- to 7-membered nitrogen-containing non-aromatic heterocyclyl; provided that the compound is not of the formula: 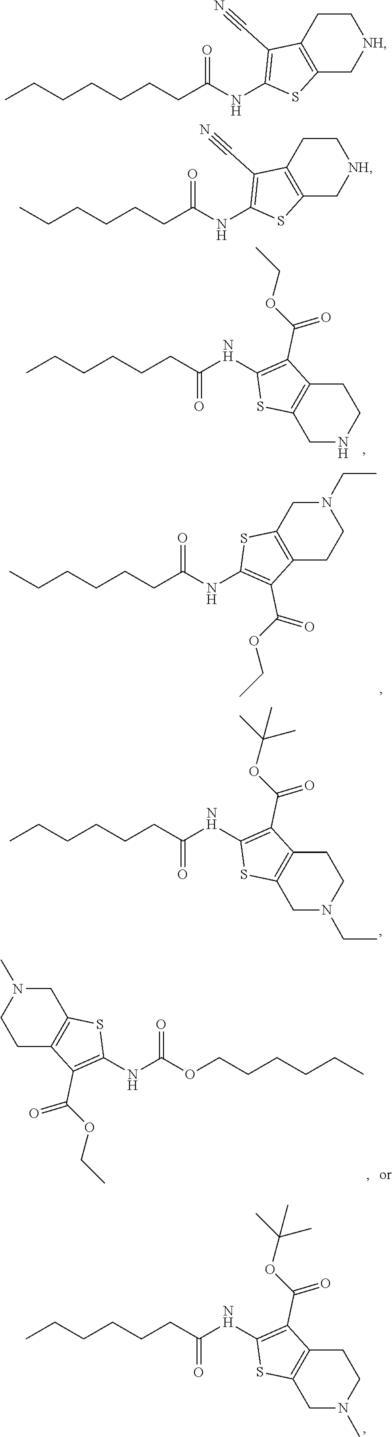
or a salt thereof.

25. The compound of claim 24, wherein the compound has a formula II′: 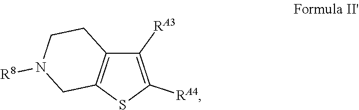
wherein:
R⁸ is H or R^{c}; and
R^{c} is -aliphatic, -aliphatic-amino, —C(O)-aliphatic, or —C(O)-aliphatic-amino.

26. The compound of claim 24, wherein the compound has a formula II: 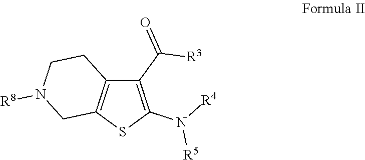
wherein:
R⁸ is H or Re; and
R^{c} is -aliphatic, -aliphatic-amino, —C(O)-aliphatic, or —C(O)-aliphatic-amino.

27. The compound of claim 26, wherein the compound has a formula II-a:

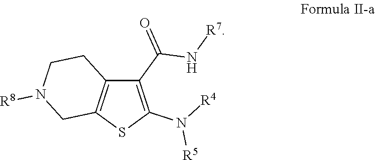

28. The compound of claim 25, wherein R^{c} is —C₆₋₃₀aliphatic, —C₁₋₆aliphatic-amino, —C(O)—C₆₋₃₀aliphatic or —C(O)—C₁₋₆aliphatic-amino.

29. The compound of claim 25, wherein R^{c} is —(CH₂)₁₋₄N(R^{a})₂ where each R^{a} independently is H or C₁₋₆alkyl, or both R^{a}s together with the nitrogen to which they are attached form an optionally substituted 5- or 6-membered heterocyclyl.

30. The compound of claim 28, wherein R^{c} is —C(O)—C₆₋₃₀aliphatic.

31. The compound of claim 24, wherein the compound has a Formula III′:

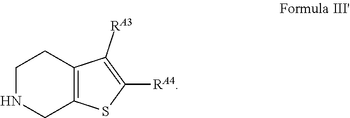

32. The compound of claim 24, wherein the compound has a Formula III:

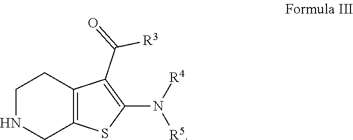

33. The compound of claim 1, wherein R³ is —O—C₆₋₃₀aliphatic.

34. The compound of claim 33, wherein R³ is —O—C₁₆₋₂₀aliphatic.

35. The compound of claim 34, wherein R³ is —O—C₁₆₋₂₀alkyl.

36. The compound of claim 34, wherein R³ is —O—C₁₆₋₂₀alkenyl.

37. The compound of claim 1, wherein R³ is —O—C₁₋₈aliphatic.

38. The compound of claim 37, wherein R³ is —O—C₁₋₆alkyl.

39. The compound of claim 1, wherein R⁴ is —C(O)—C₆₋₃₀aliphatic.

40. The compound of claim 39, wherein R⁴ is —C(O)—C₁₆₋₂₀aliphatic.

41. The compound of claim 1, wherein R⁵ is H.

42. The compound of claim 1, wherein R⁵ is —C(O)—C₆₋₃₀aliphatic.

43. The compound of claim 24, wherein the compound has a Formula IV′ 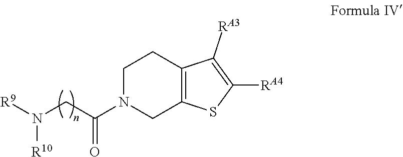
wherein:
each of R⁹ and R¹⁰ independently is H, C₁₋₆alkyl, or R⁹ and R¹⁰ together with the nitrogen to which they are attached, form an optionally substituted 5- or 6-membered non-aromatic heterocyclyl moiety; and
n is an integer from 1 to 8.

44. The compound of claim 24, wherein the compound has a Formula IV 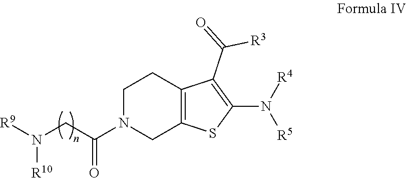
wherein:
each of R⁹ and R¹⁰ independently is H, C₁₋₆alkyl, or R⁹ and R¹⁰ together with the nitrogen to which they are attached, form an optionally substituted 5- or 6-membered non-aromatic heterocyclyl moiety; and
n is an integer from 1 to 8.

45. The compound of claim 43, wherein each of R⁹ and R¹⁰ independently is C₁₋₆alkyl.

46. The compound of claim 43, wherein n is 2 or 3.

47. The compound of claim 43, wherein R³ is —O—C₁₋₈aliphatic.

48. The compound of claim 43, wherein R³ is N(R⁶)(R⁷).

49. The compound of claim 1, wherein R³ is —NH—(CH₂)ₚ—N(R^{a})₂ or
—N[(CH₂)ₚ—N(R^{a})₂]₂, optionally wherein at least one —CH₂— of —(CH₂)P— is replaced with arylene or heteroarylene;
each R^{a} independently is H or C₁₋₆alkyl, or both R^{a}s together with the nitrogen to which they are attached form an 5- or 6-membered heterocyclyl; and
each p is independently an integer from 1 to 8.

50. The compound of claim 1, wherein R³ is N(R⁶)(R⁷), wherein R⁶ and R⁷, together with the atom to which there are attached, form a 4- to 7-membered nitrogen-containing heterocyclyl.

51. The compound of claim 50, wherein R³ is N(R⁶)(R⁷), wherein R⁶ and R⁷, together with the atom to which there are attached, form piperazinyl.

52. The compound of claim 1, wherein R⁶ is C₆₋₃₀aliphatic.

53. The compound of claim 52, wherein R⁶ is C₁₆₋₂₀aliphatic.

54. The compound of claim 1, wherein R⁶ is H.

55. The compound of claim 1, wherein R⁷ is —C(O)—C₆₋₃₀aliphatic.

56. The compound of claim 55, wherein R⁷ is —C(O)—C₁₆₋₂₀aliphatic.

57. The compound of claim 1, wherein R⁷ is C₆₋₃₀aliphatic.

58. The compound of claim 24, wherein the compound has a Formula V′ 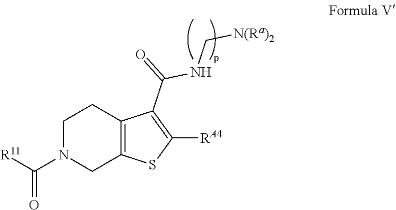
wherein:
R¹¹ is C₆₋₃₀aliphatic;
each R^{a} independently is H or C₁₋₆alkyl, or both R^{a}s together with the nitrogen to which they are attached form an optionally substituted 5- or 6-membered heterocyclyl; and
p is an integer from 1 to 8.

59. The compound of claim 24, wherein the compound has a Formula V 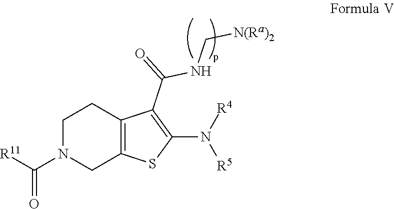
wherein:
R¹¹ is C₆₋₃₀aliphatic;
each R^{a} independently is H or C₁₋₆alkyl, or both R^{a}s together with the nitrogen to which they are attached form an optionally substituted 5- or 6-membered heterocyclyl; and
p is an integer from 1 to 8.

60. The compound of claim 48, wherein p is 1, 2, 3, or 4.

61. The compound of claim 58, wherein R¹¹ is —C₁₅₋₂₀aliphatic.

62. The compound of claim 44, wherein R⁴ is —C(O)—C₆₋₃₀aliphatic.

63. The compound of claim 62, wherein R⁴ is —C(O)—C₁₅₋₂₀aliphatic.

64. The compound of claim 44, wherein R⁵ is H.

65. The compound of claim 44, wherein R⁵ is —C(O)—C₆₋₃₀aliphatic.

66. The compound of claim 65, wherein R⁵ is —C(O)—C₁₅₋₂₀aliphatic.

67. The compound of claim 1, wherein the compound is of the formula: 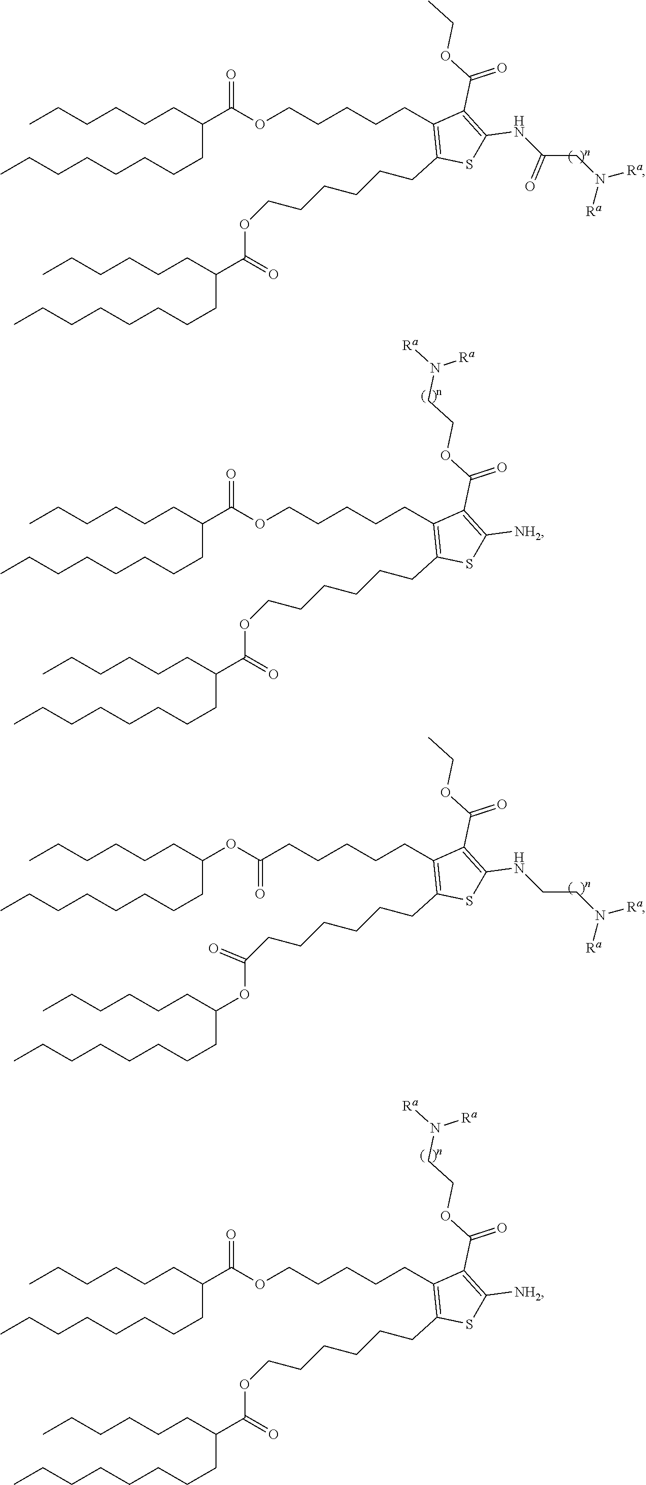 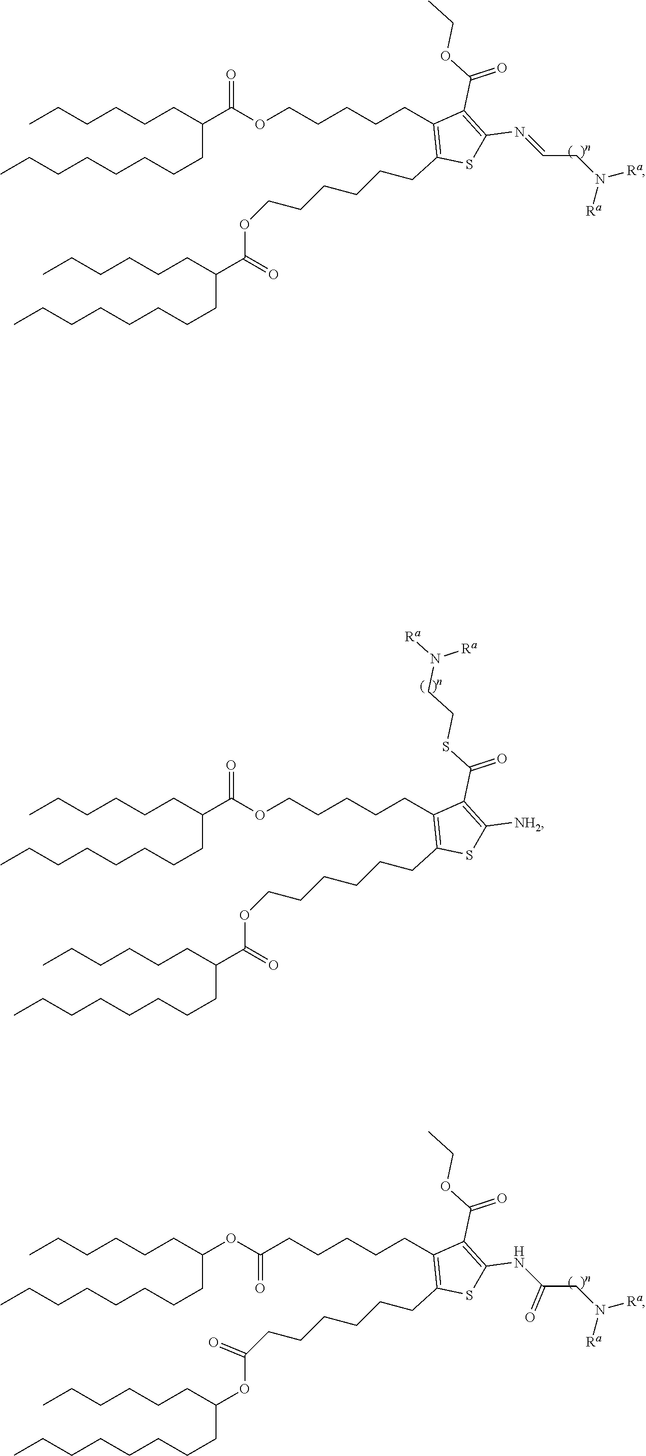 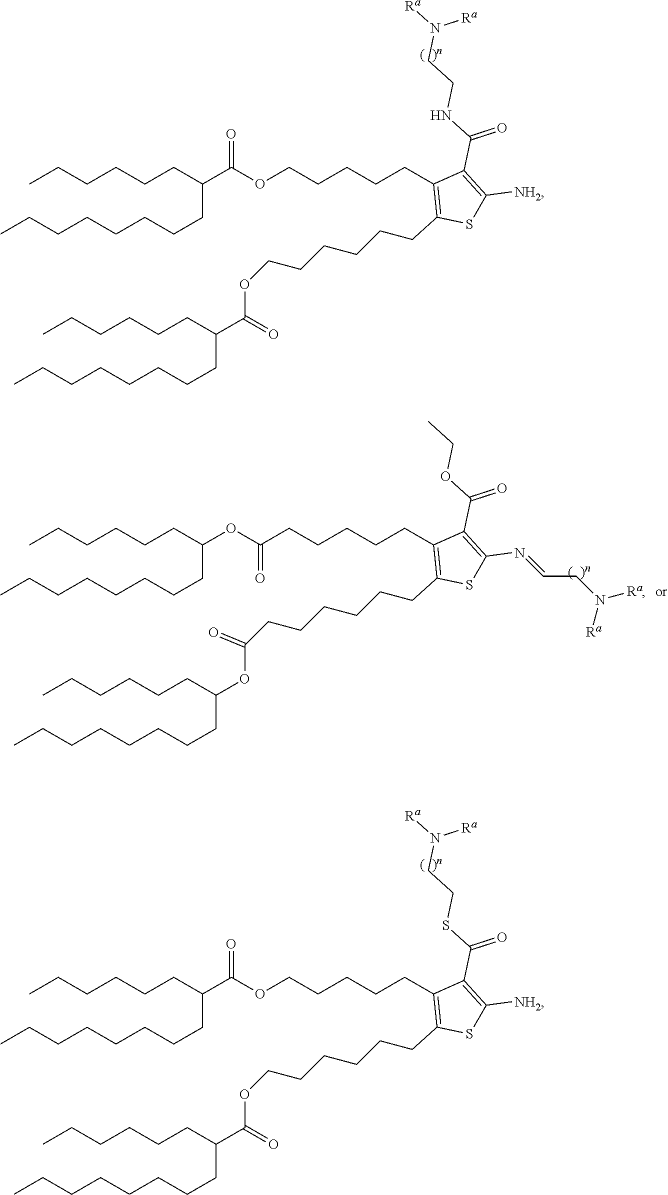
where n is from 0 to 6.

68. The compound of claim 1, wherein the compound comprises one or more basic nitrogen atoms.

69. The compound of claim 68, wherein the compound comprises only one basic nitrogen atom.

70. The compound of claim 68, wherein at least one of the basic nitrogen atoms is the nitrogen atom in —C(R^{a})₂—NH₂, —C(R^{a})₂—NH—C(R^{a})₂—, or 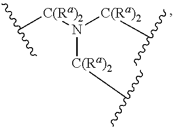
wherein each instance of R^{a} is independently H or C₁₋₆alkyl.

71. The compound of claim 8, wherein each instance of C₁₋₆aliphatic and C₁₋₆alkyl is unsubstituted C₁₋₅alkyl.

72. The compound of claim 1, wherein the compound does not comprise an acidic moiety.

73. The compound of claim 72, wherein the acidic moiety is —C(O)OH, —S(O)OH, —S(O)₂OH, or —P(O)(OH)₂.

74. The compound of claim 1, wherein the compound is of the formula:

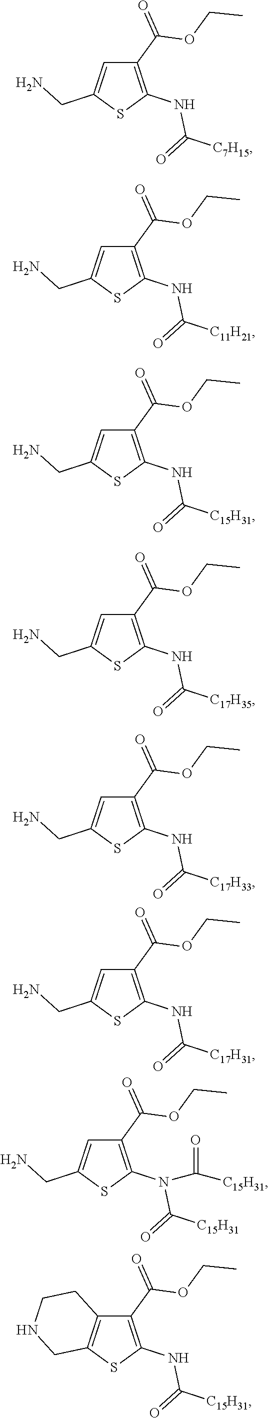

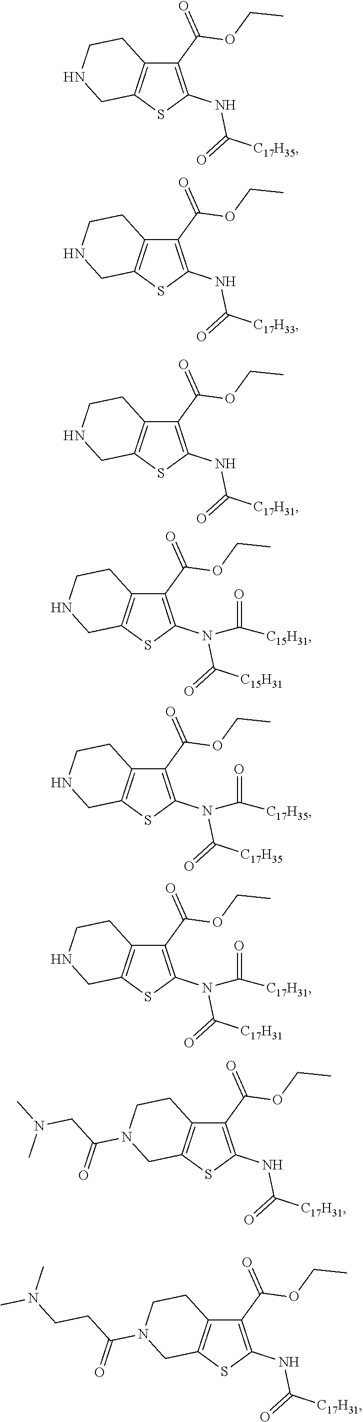

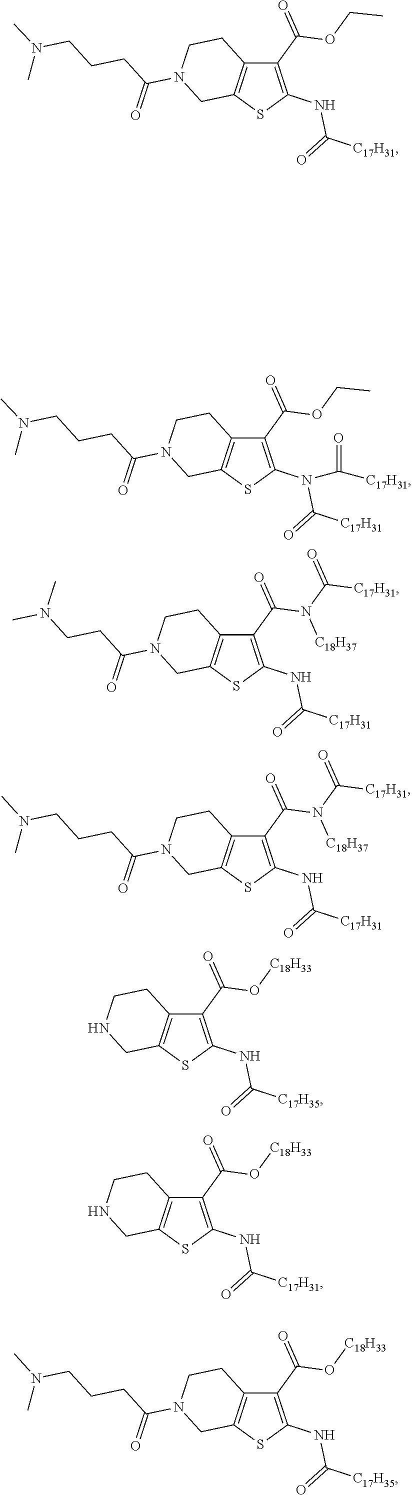

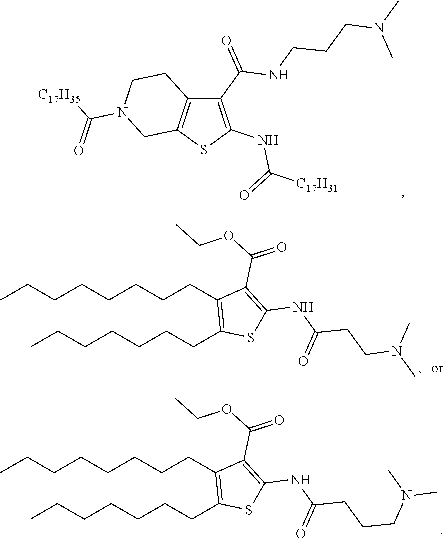

75. The compound of claim 1, wherein the compound has a Formula IX 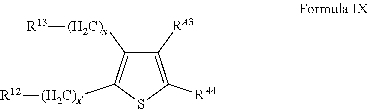
wherein:
each of R¹² and R¹³ independently is —C(O)O—R^{f}, —OC(O)—R^{f}, —C(O)NH—R^{f}, or —NH(C(O)—R^{f};
each R^{f} independently is a linear or branched C₆₋₃₀aliphatic; and
each of x and x′ independently is from 1 to 8.

76. The compound of claim 1, wherein the compound is of the formula:

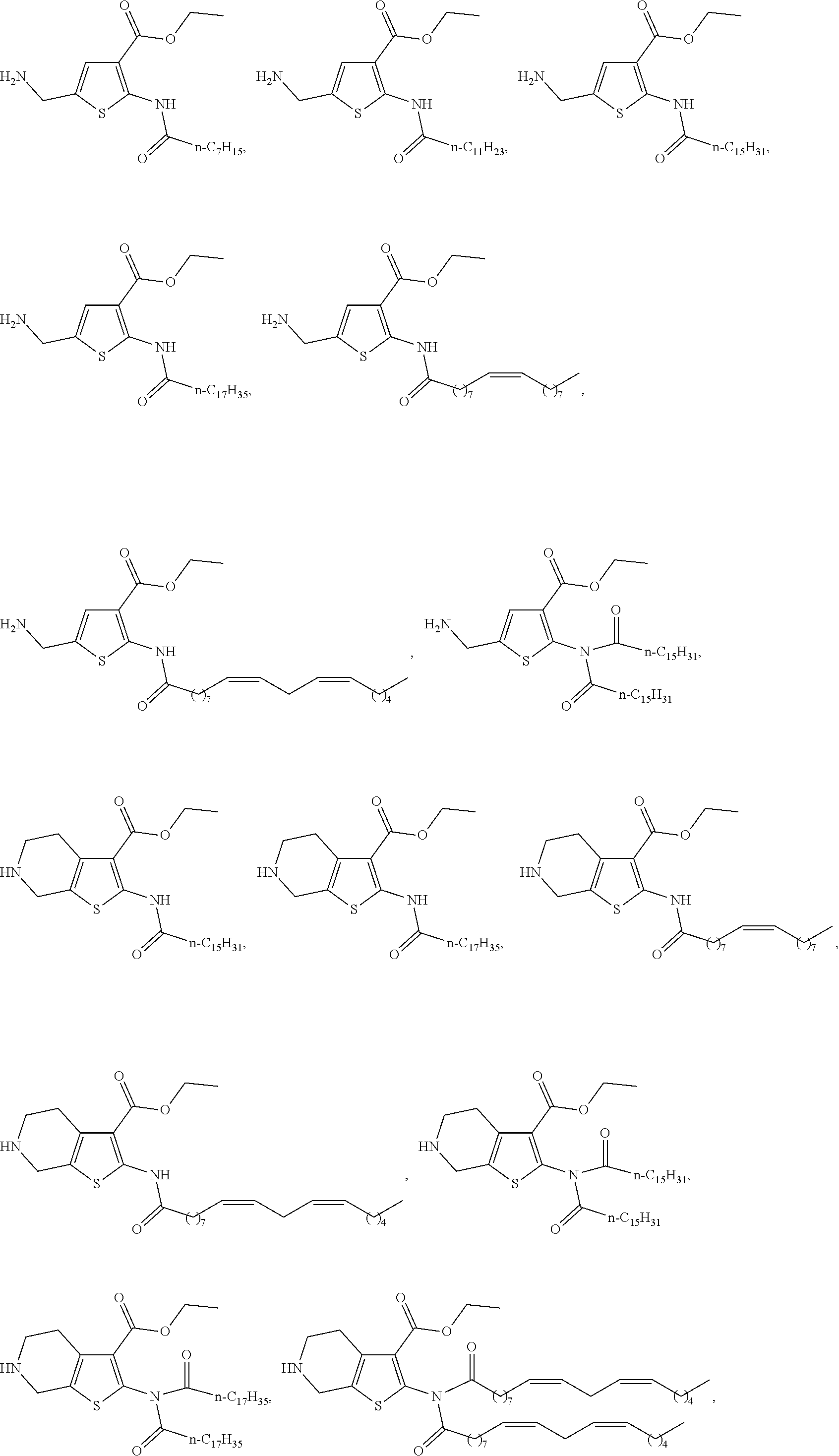

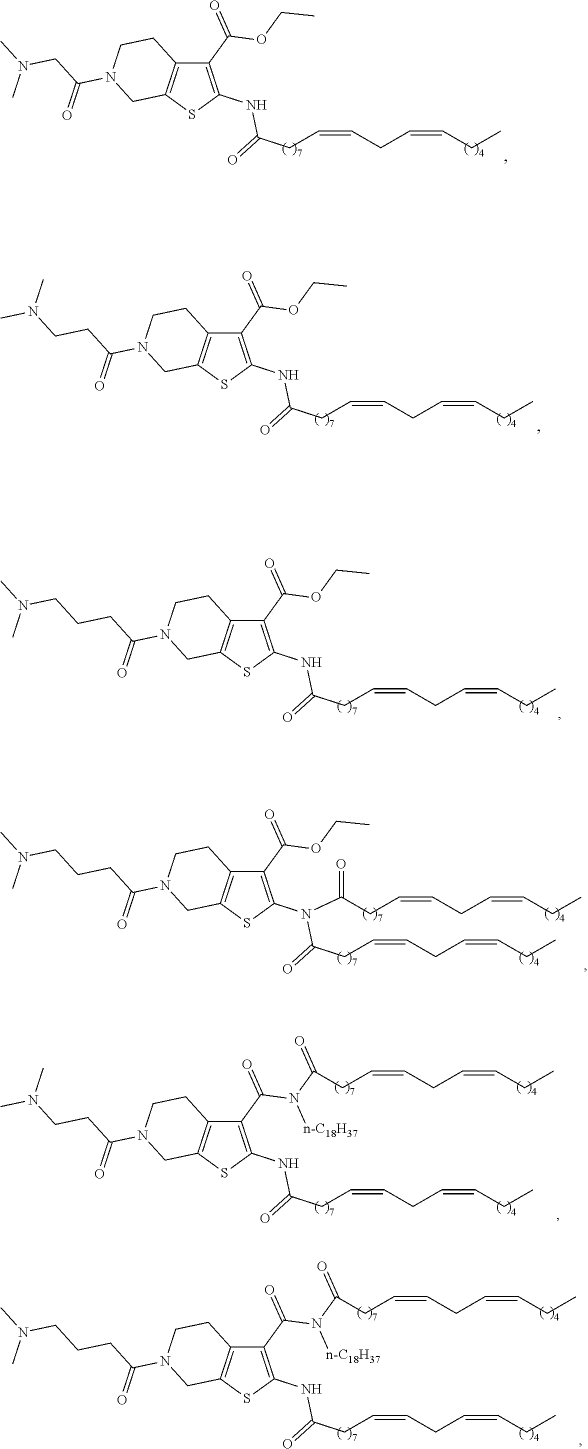

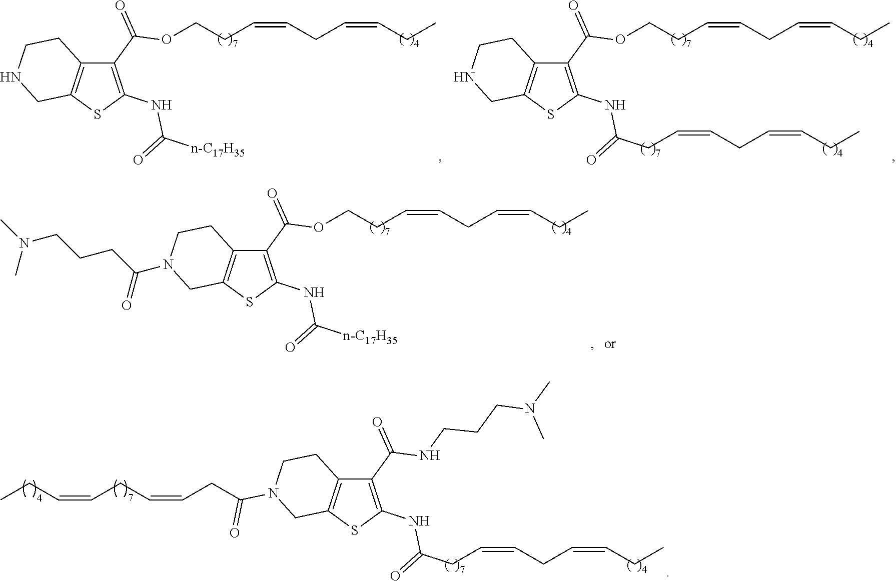

77. The compound of claim 1, wherein the compound is of the formula:

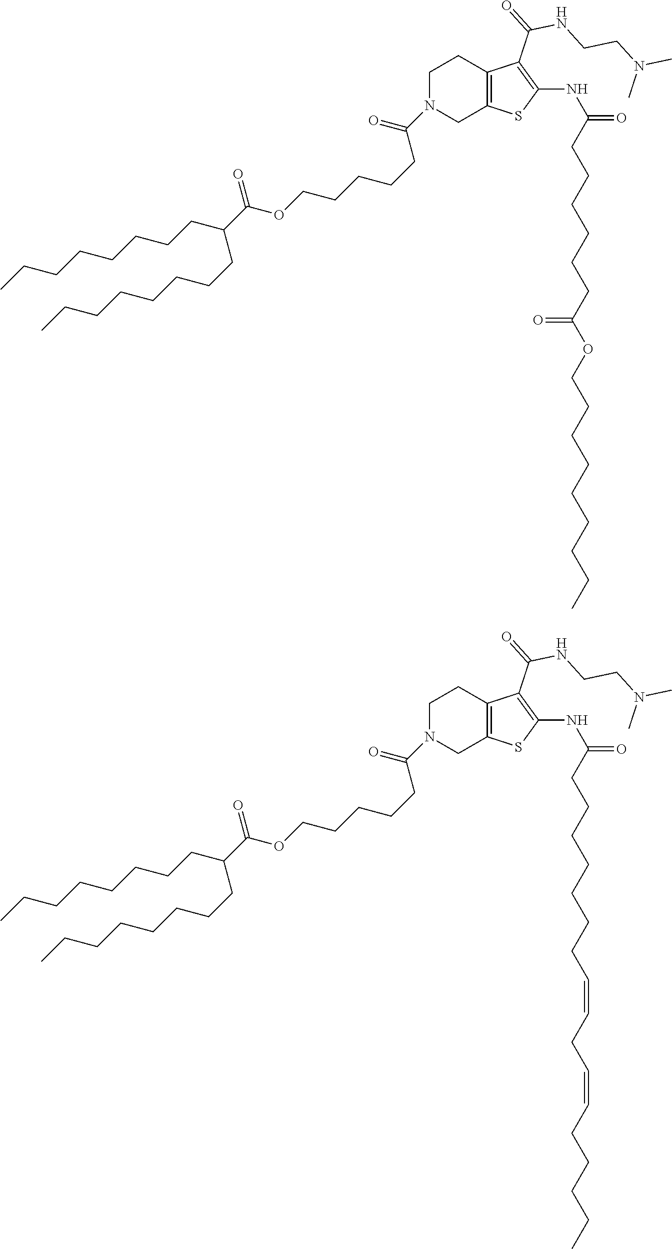

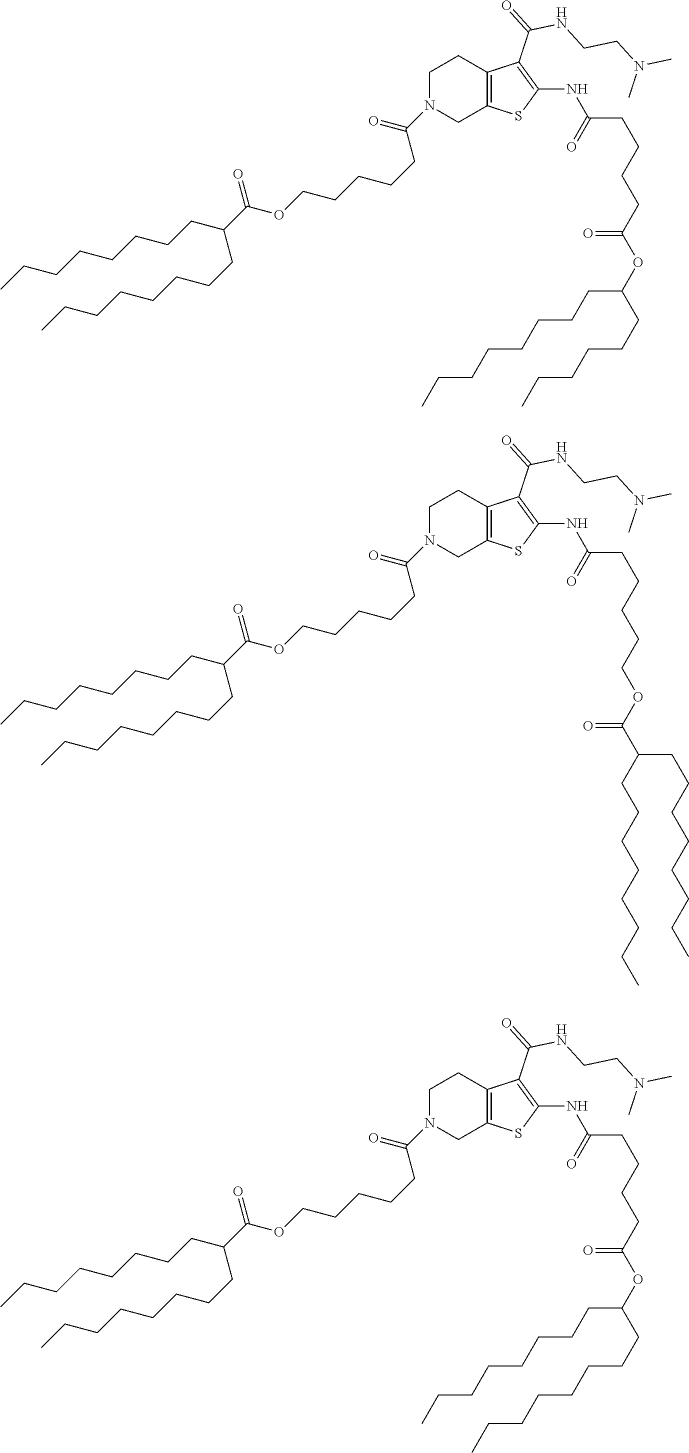

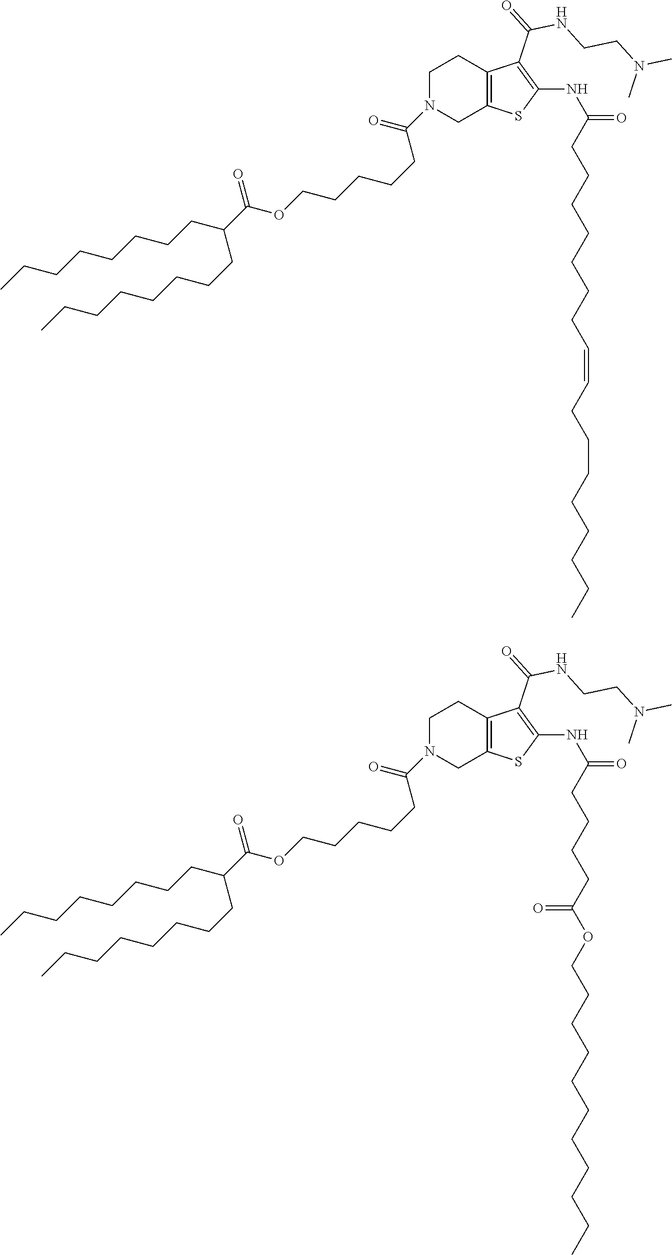

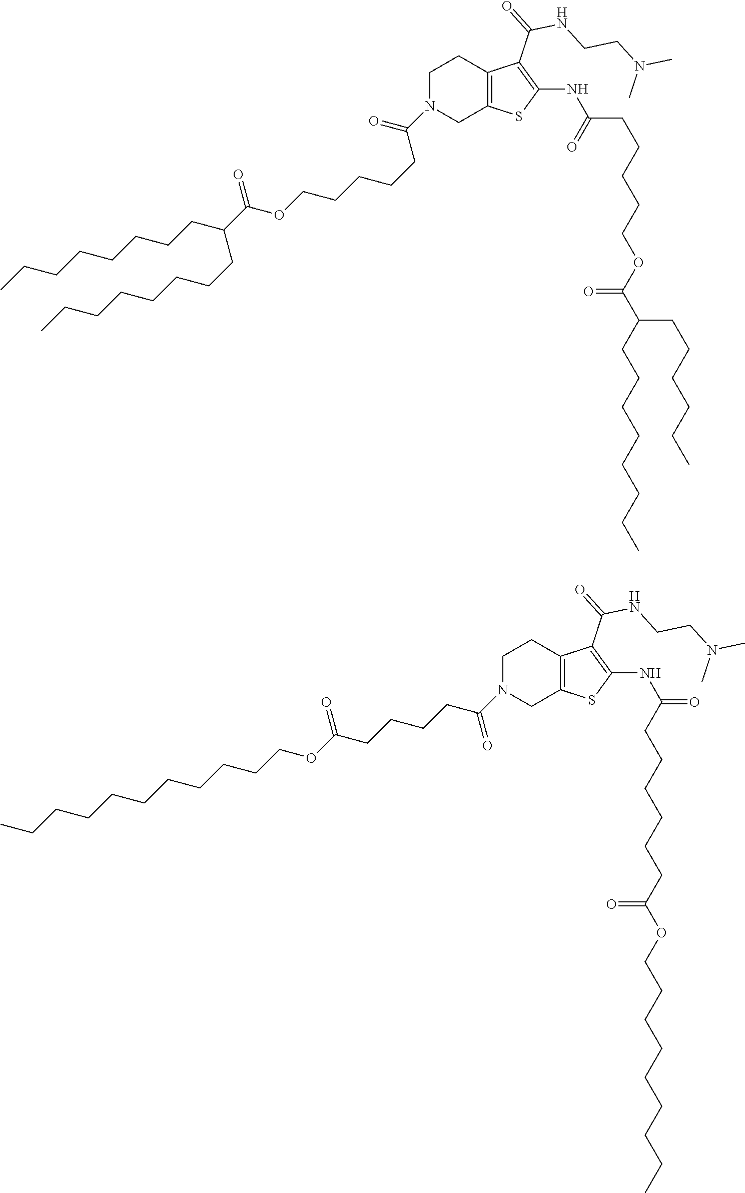

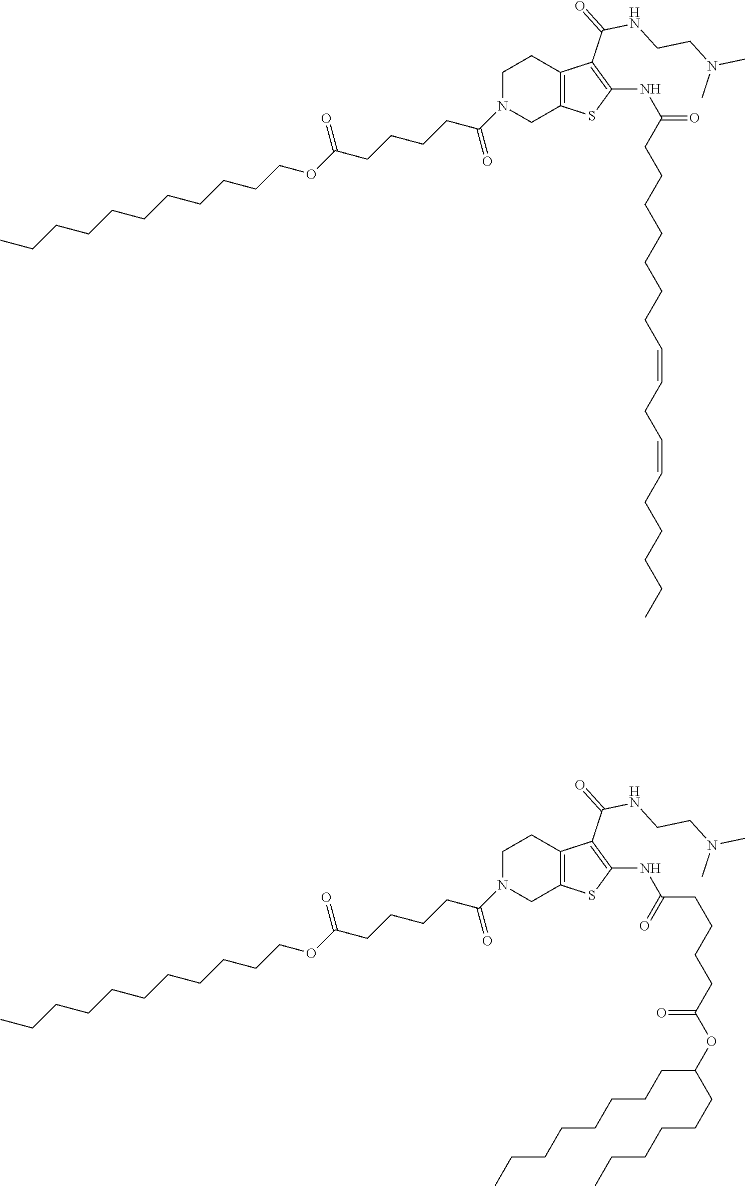

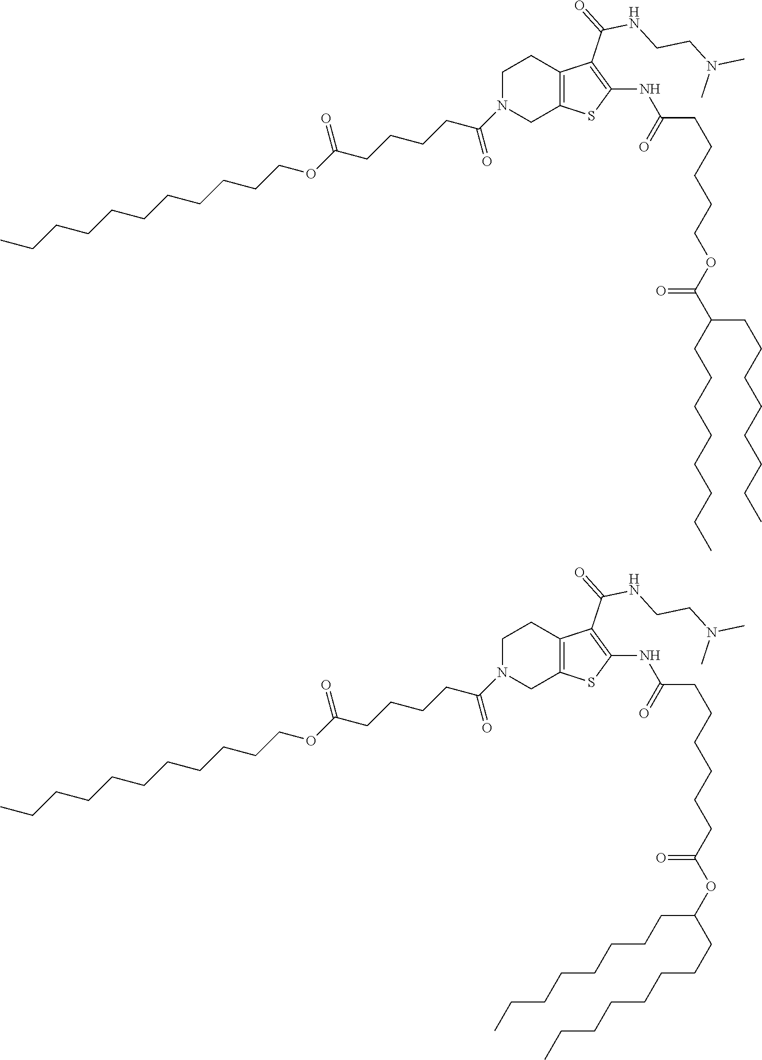

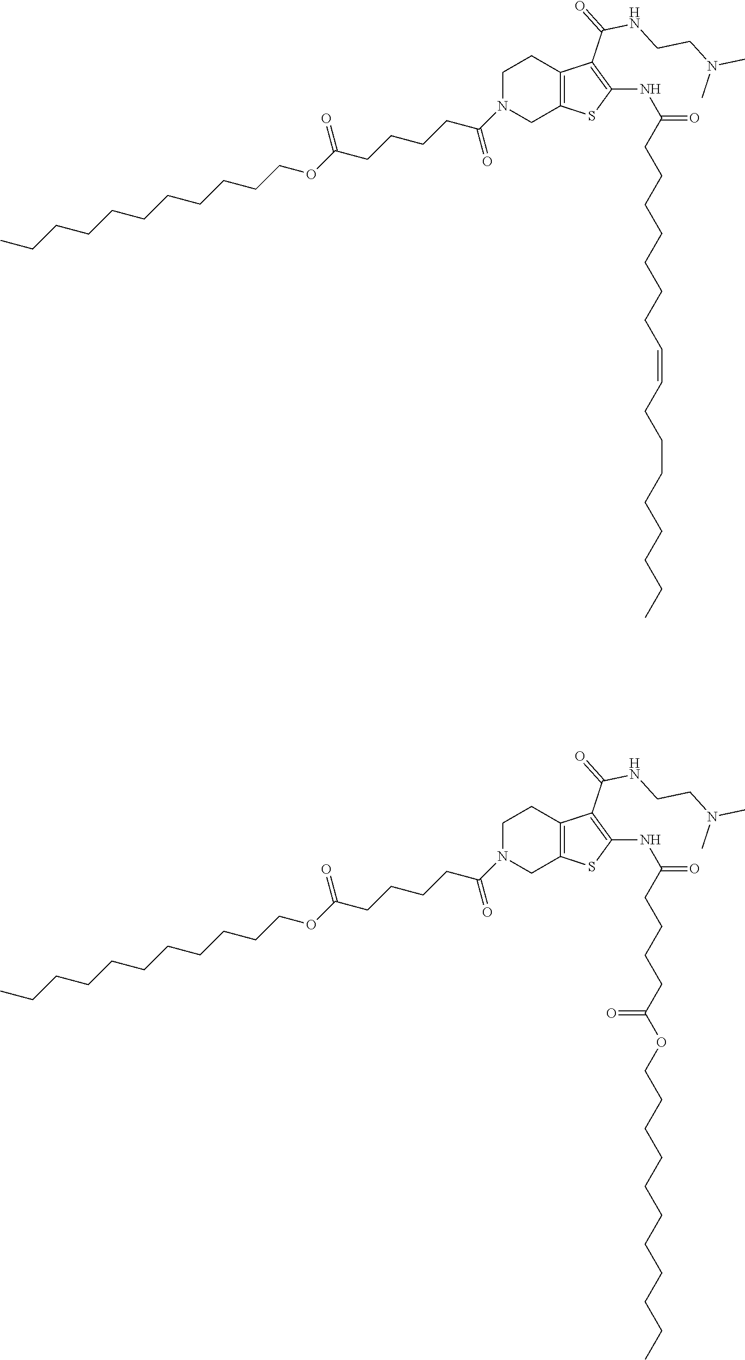

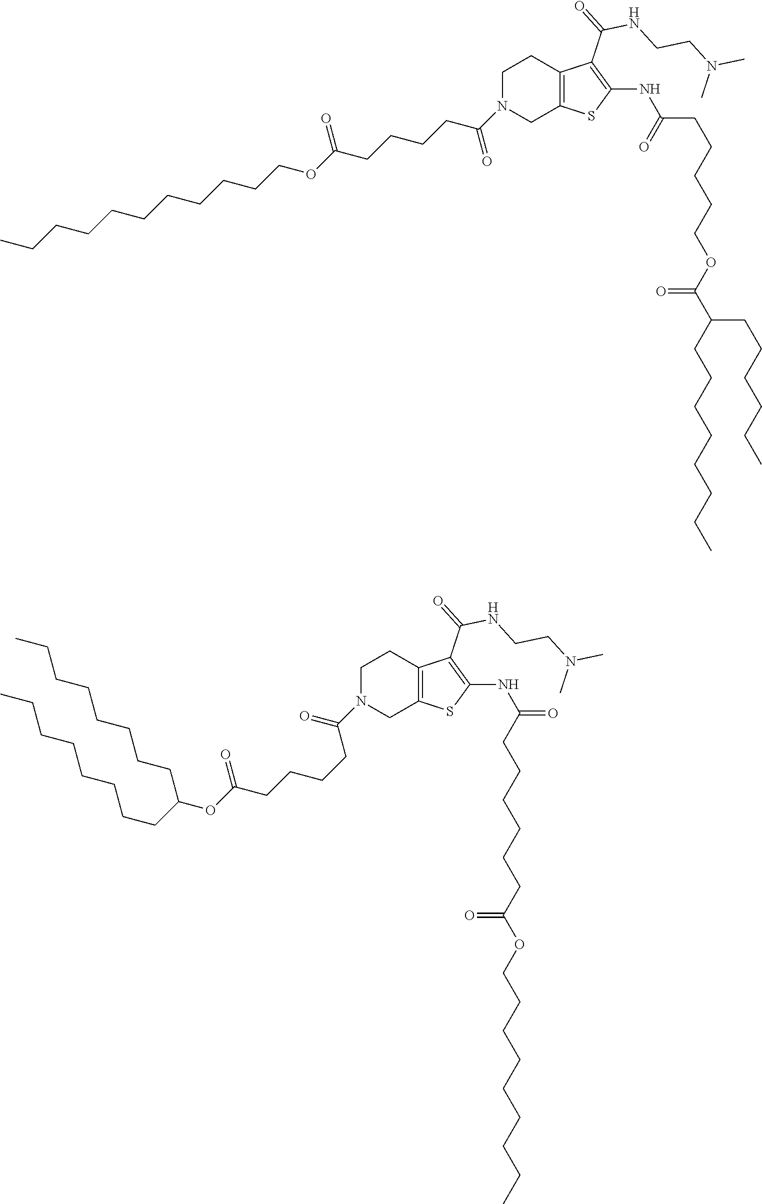

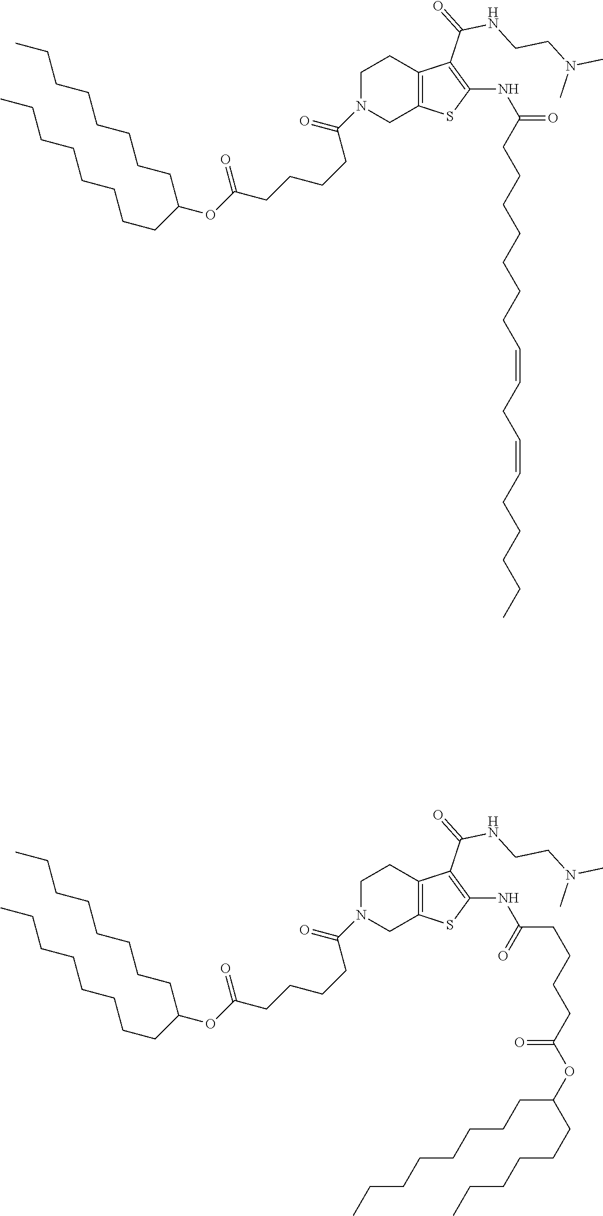

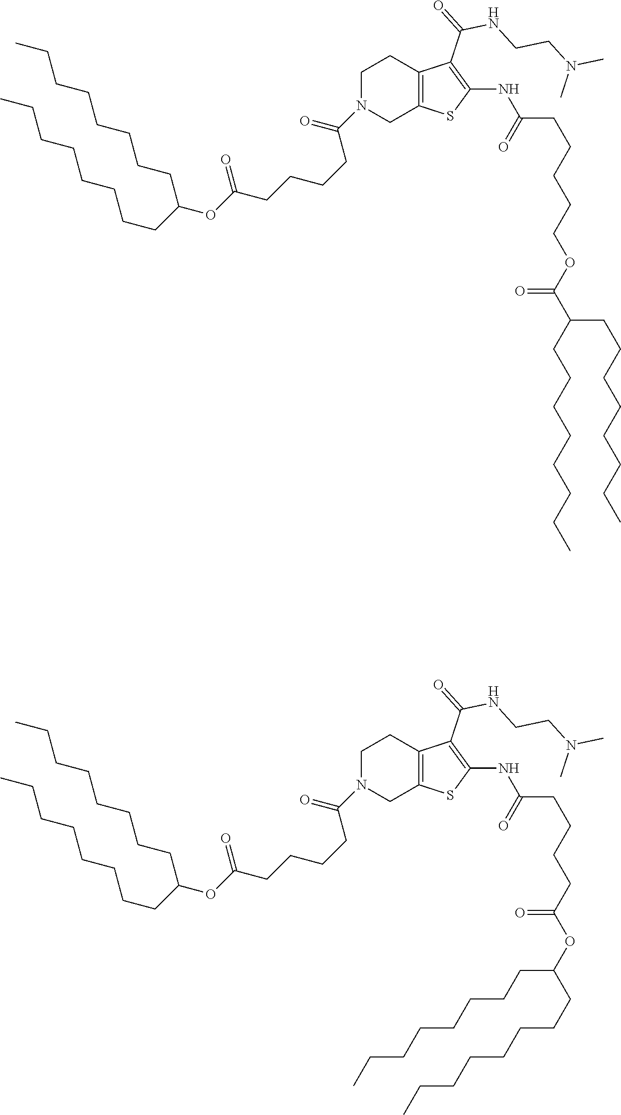

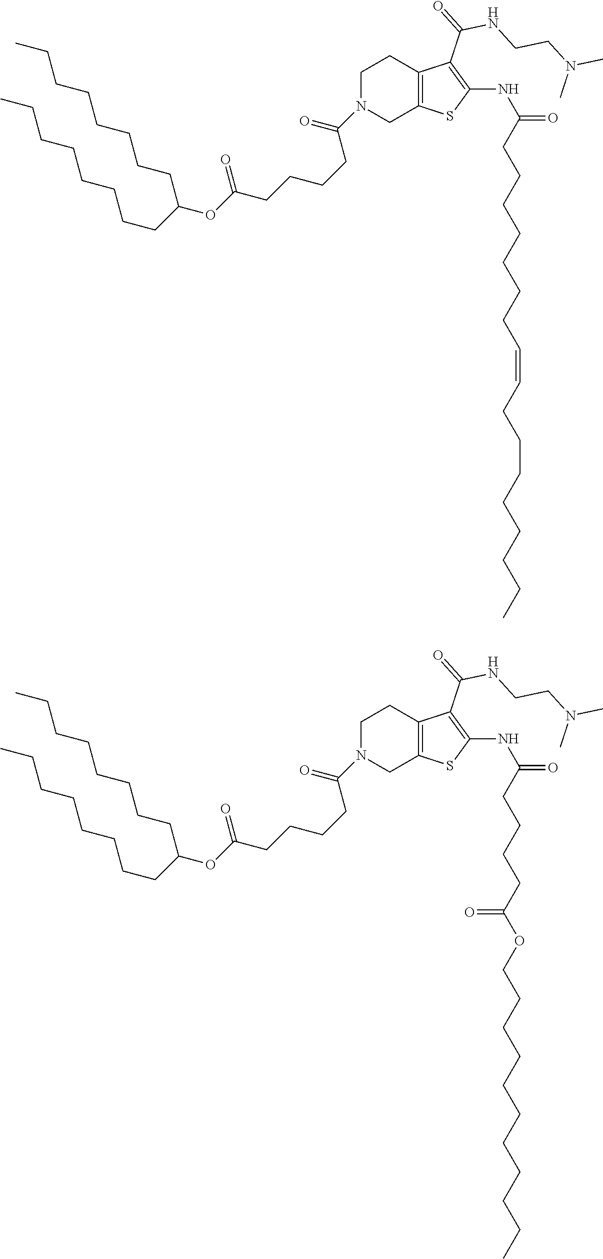

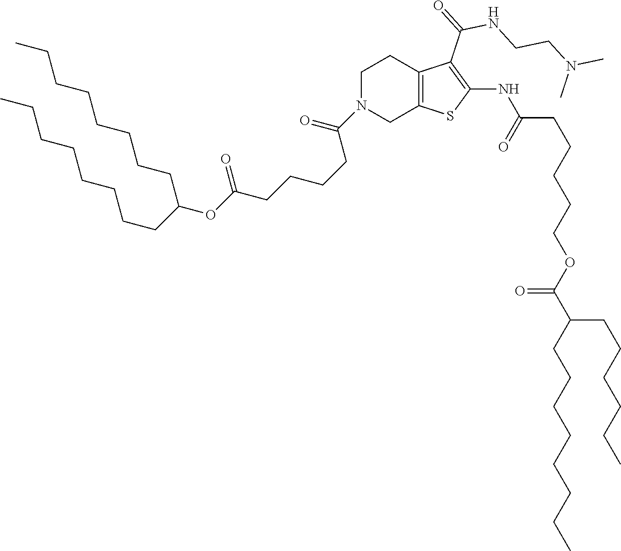 [SMILES: CCCCCCCCC(CCCCCCCC)OC(=O)CCCCC(=O)N1CCc2c(sc(NC(=O)CCCCCOC(=O)C(CCCCCC)CCCCCCCC)c2C(=O)NCCN(C)C)C1]

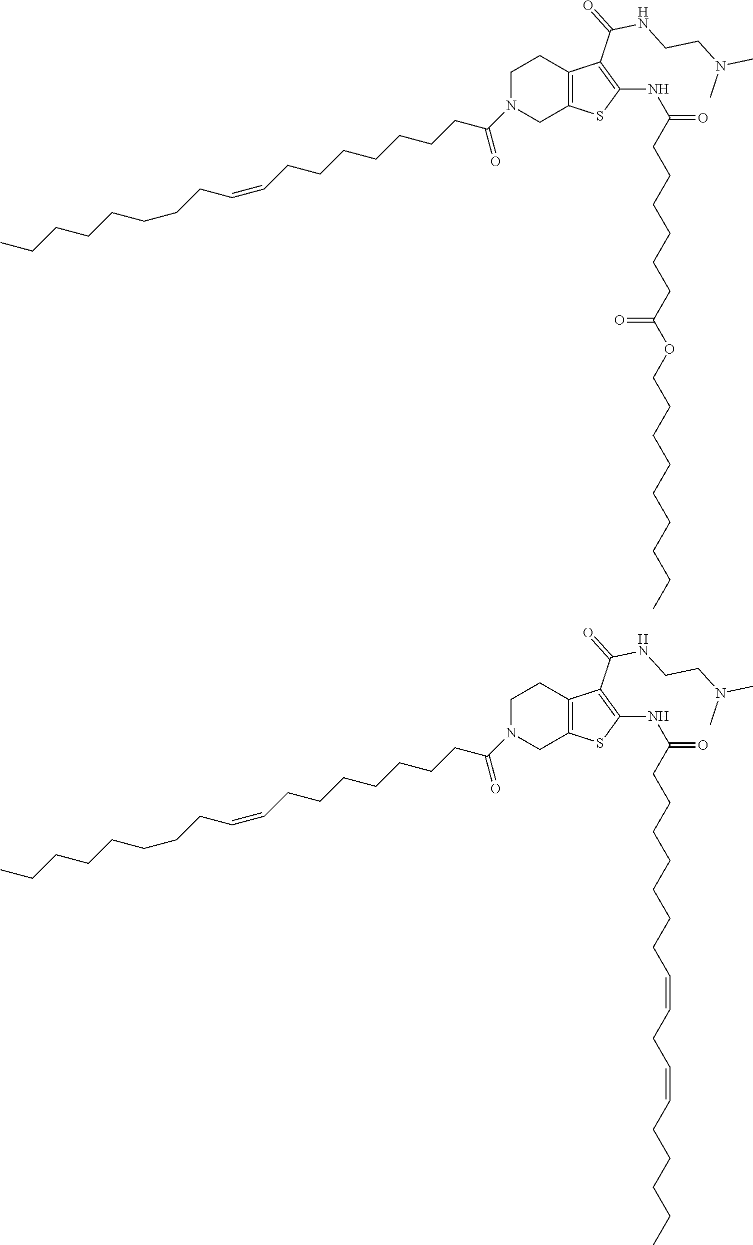

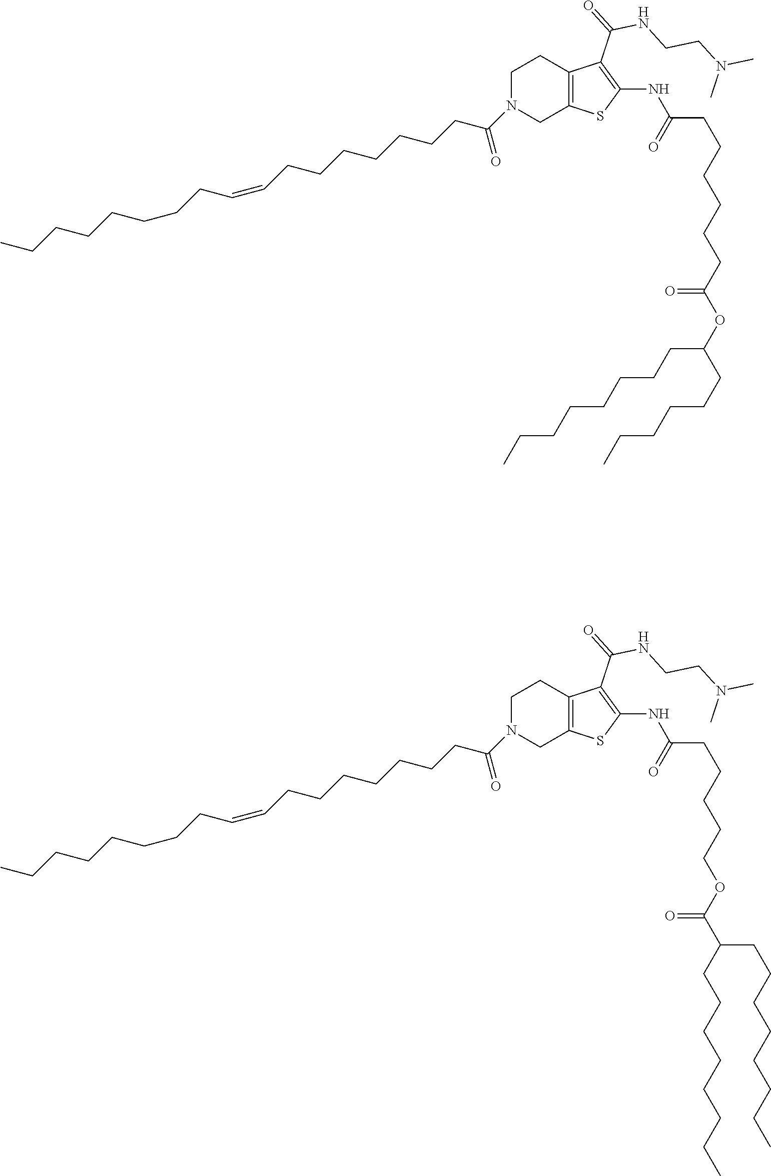

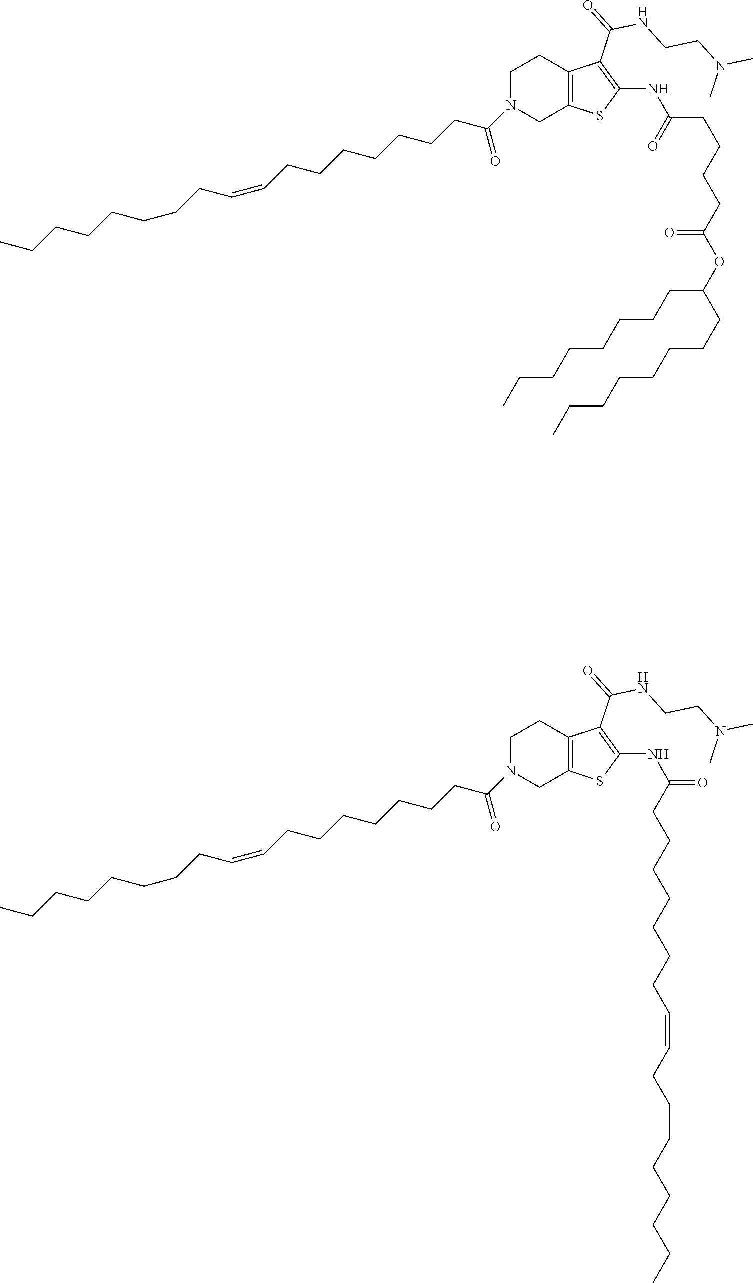

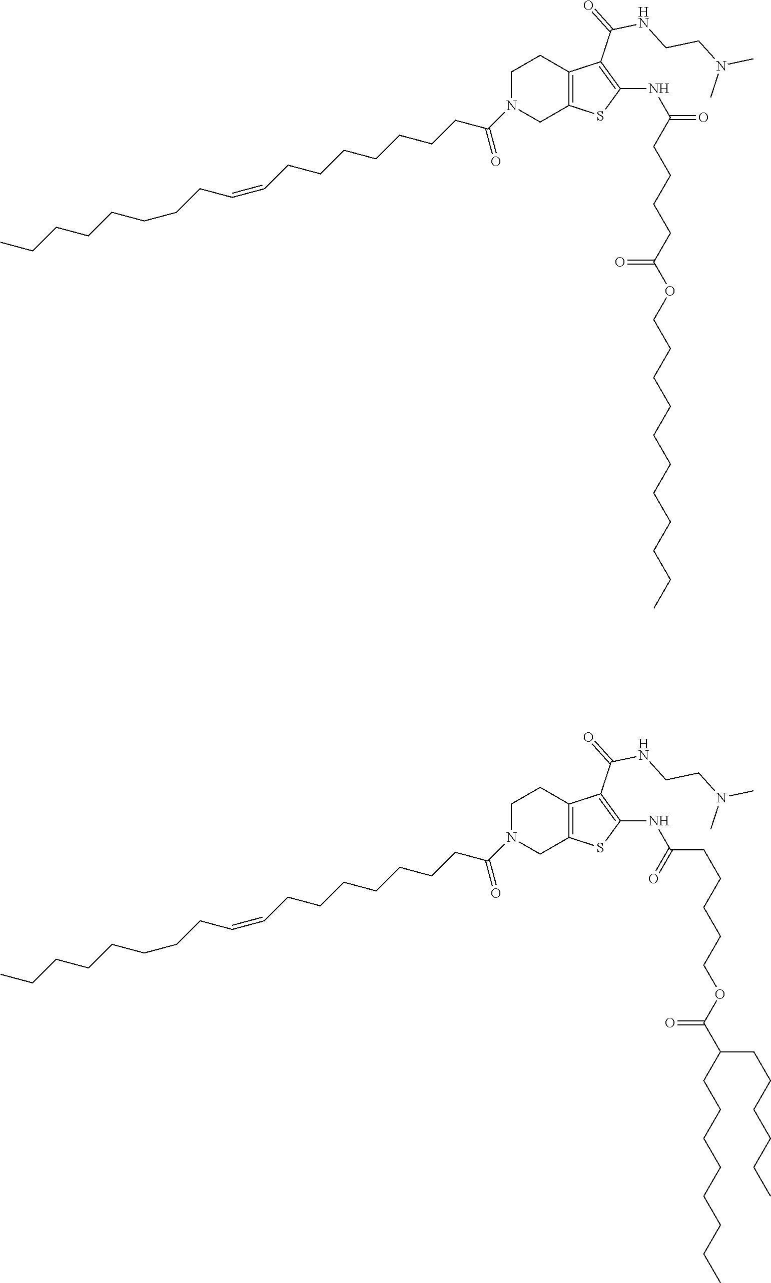

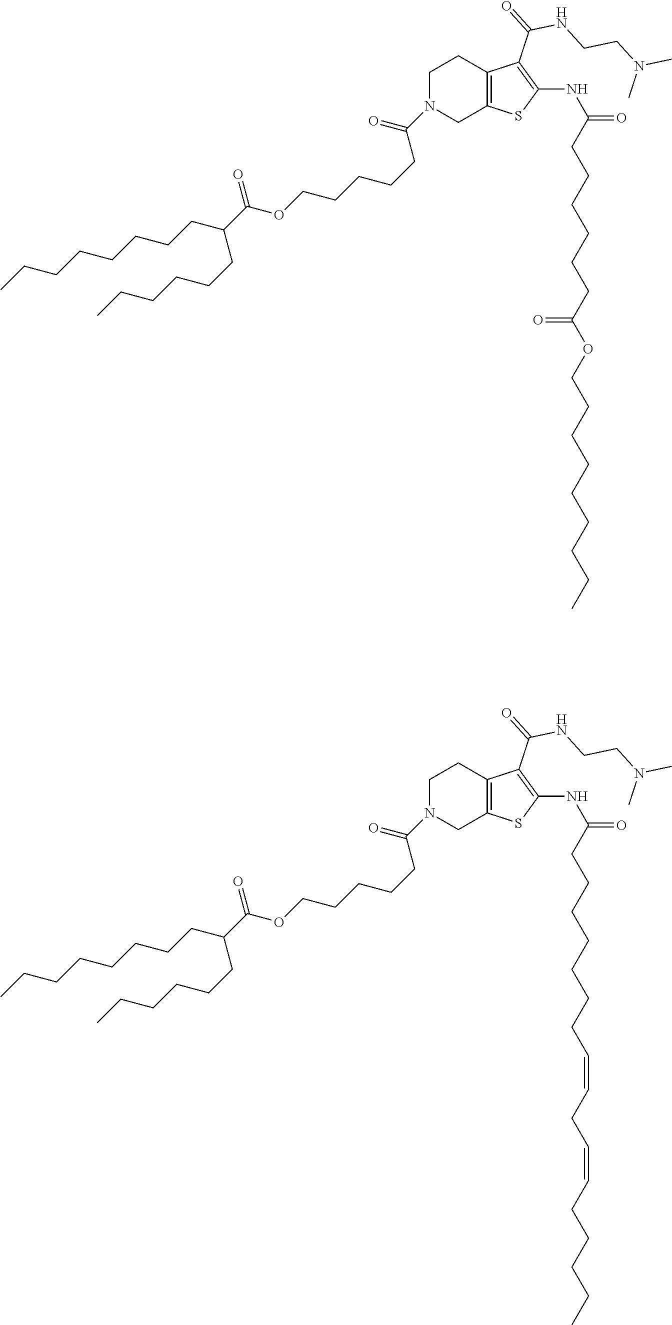

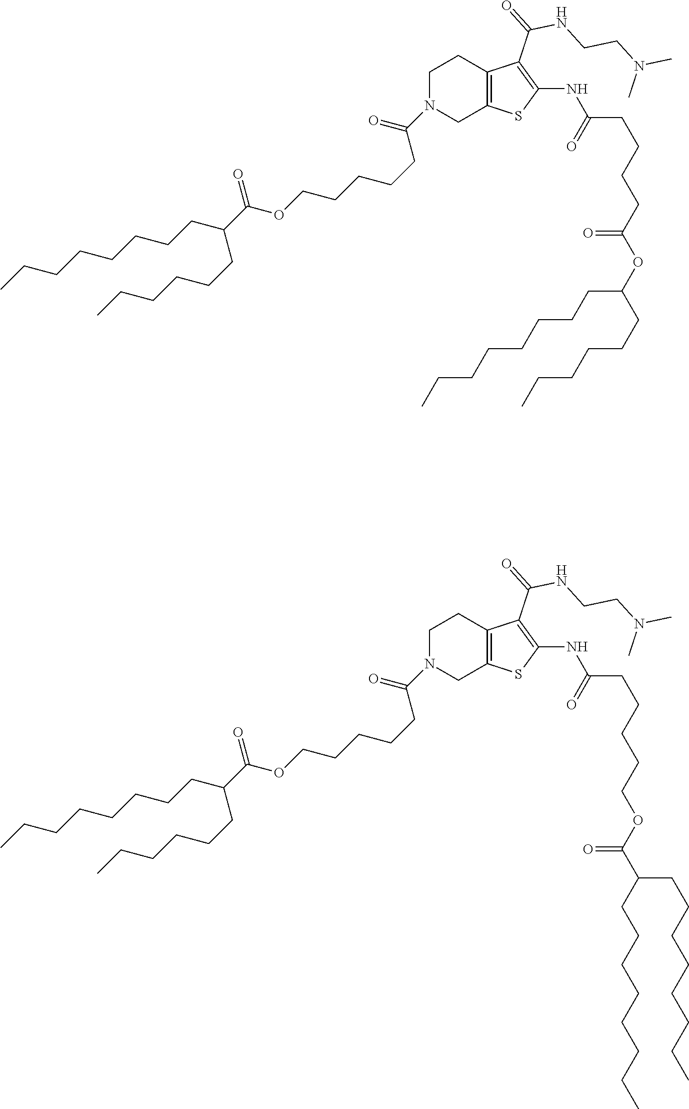

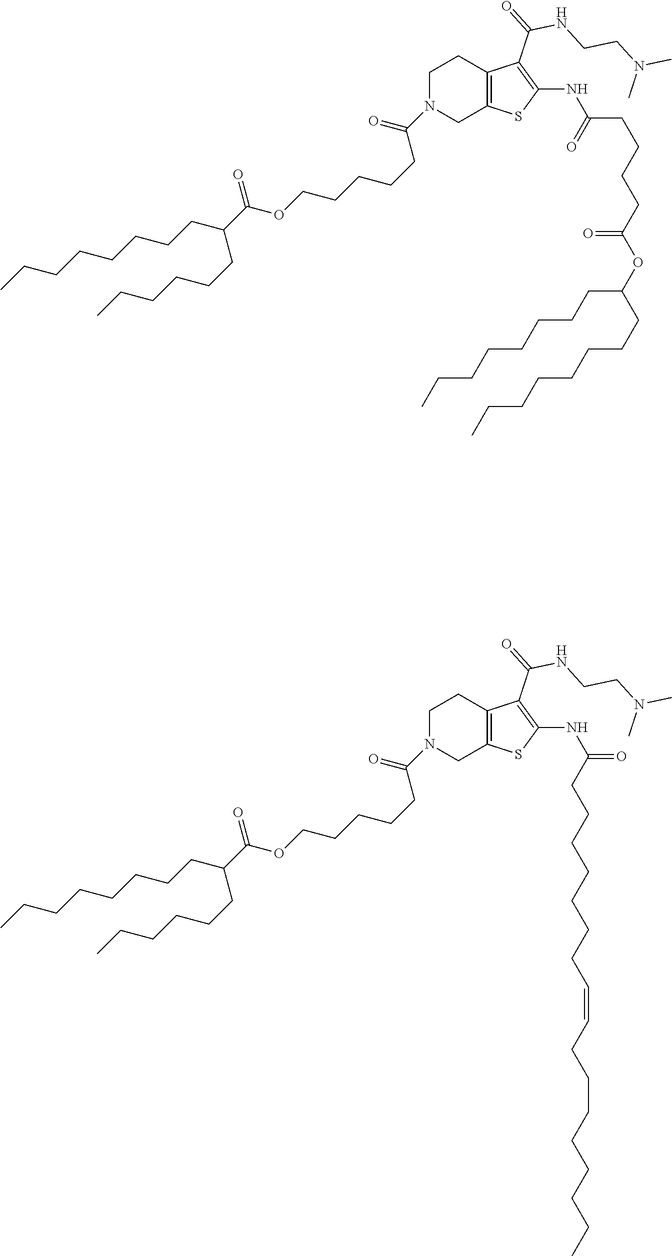

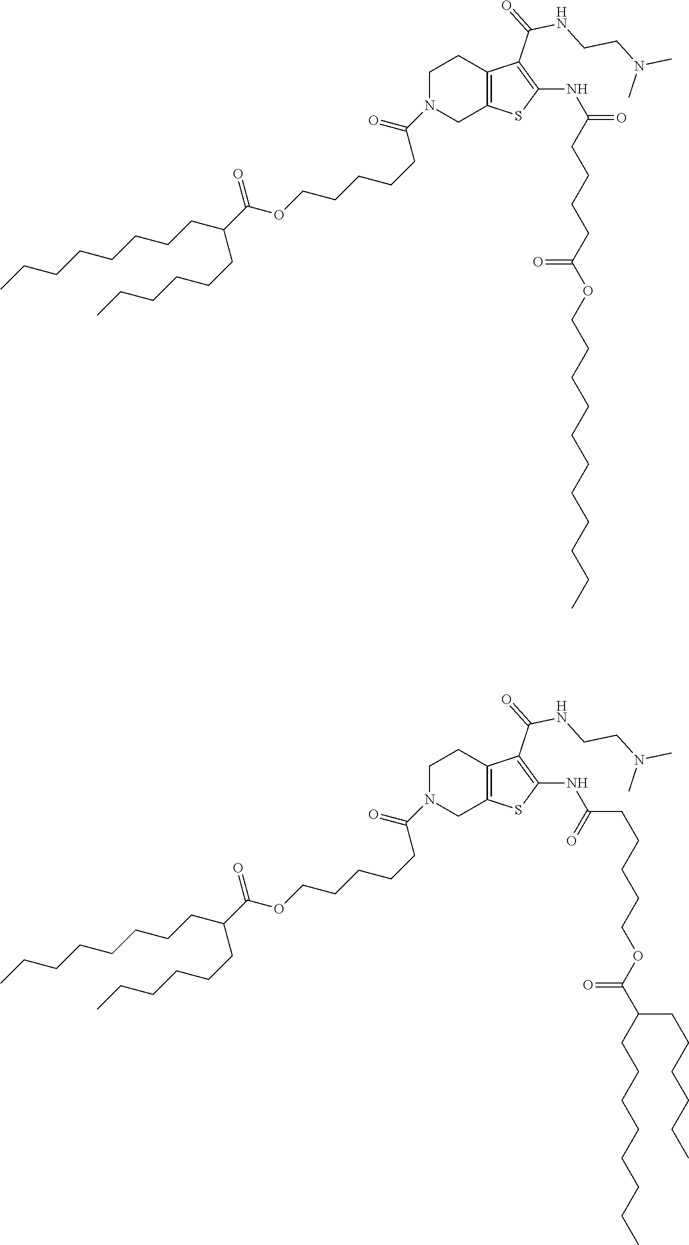

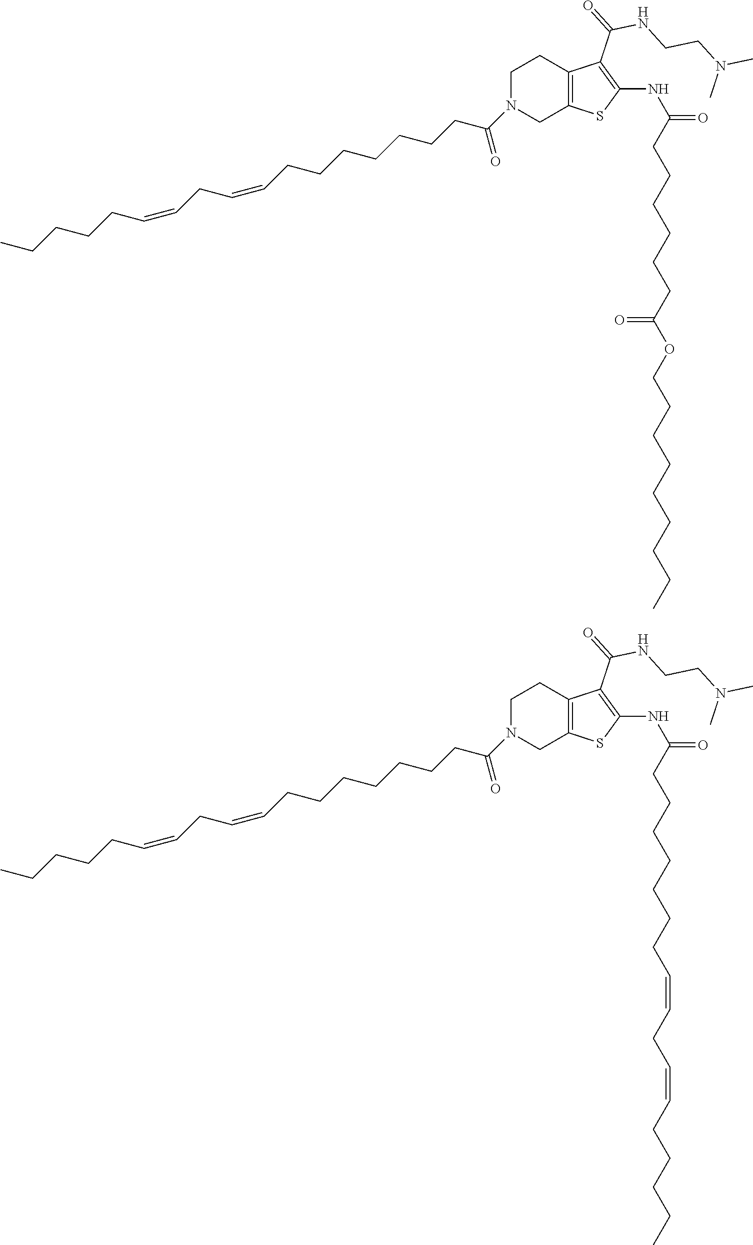

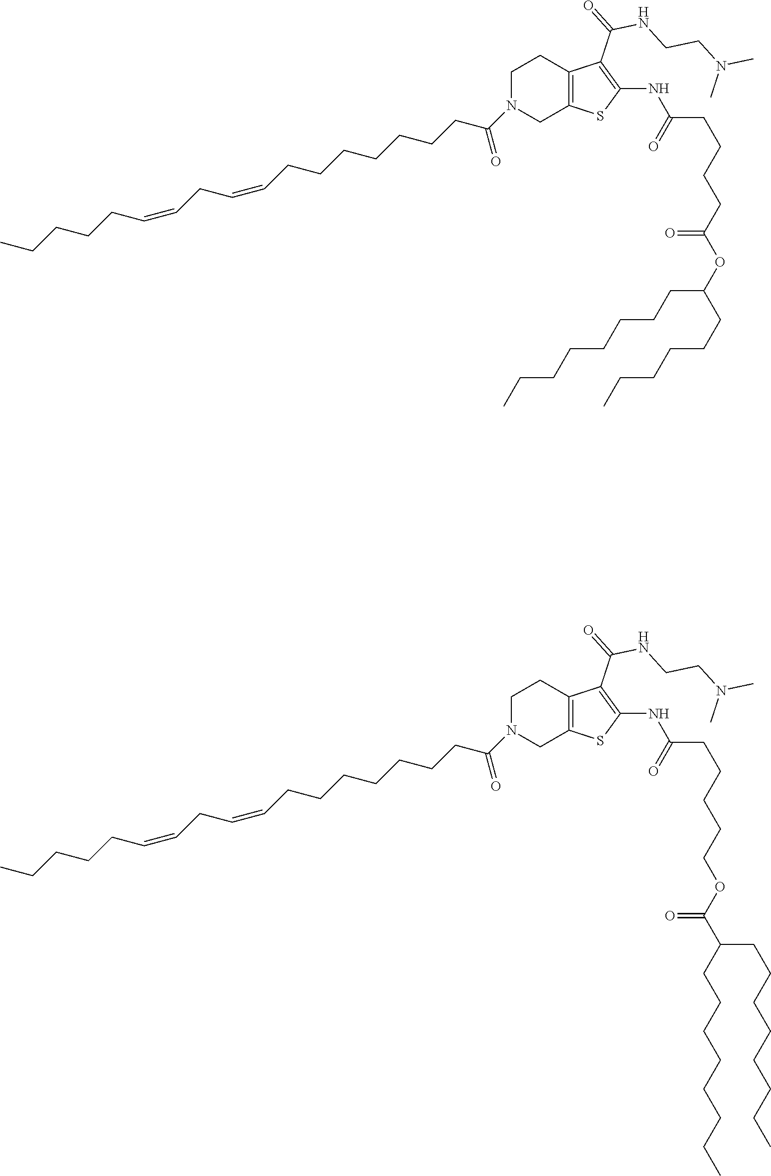

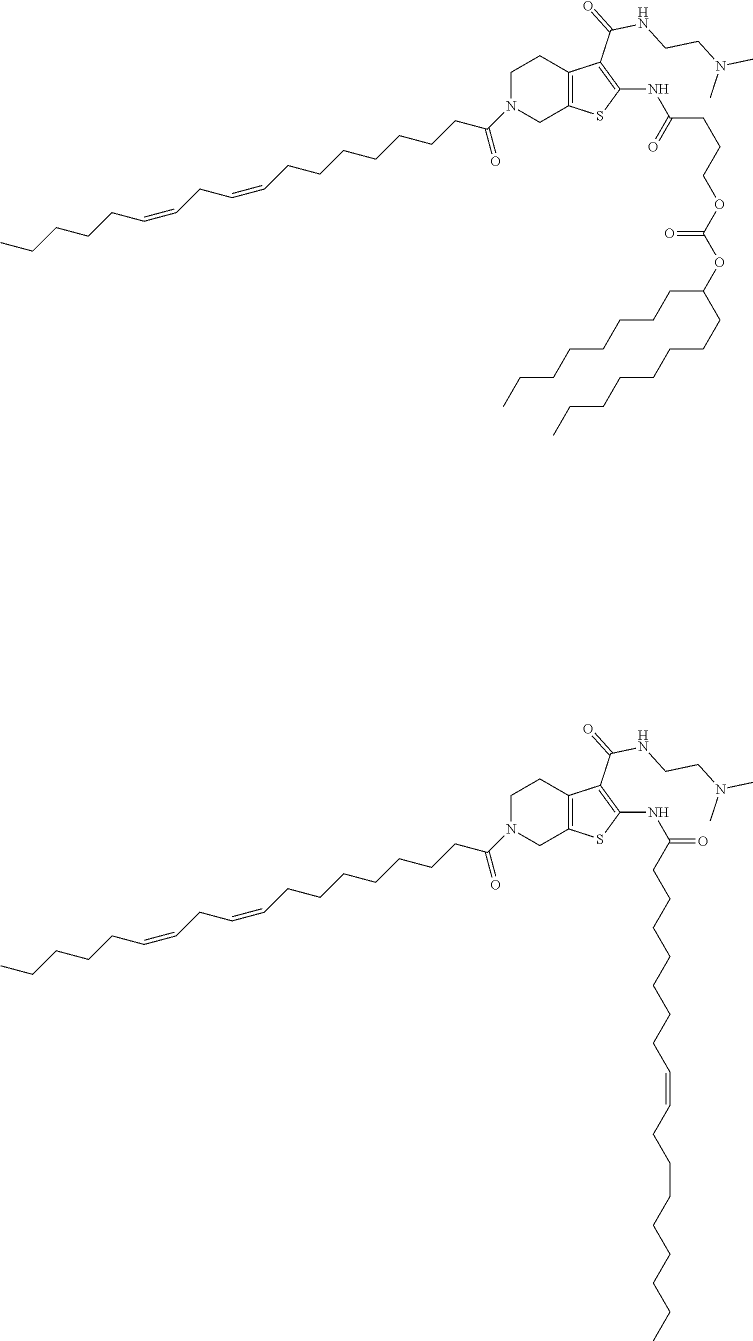

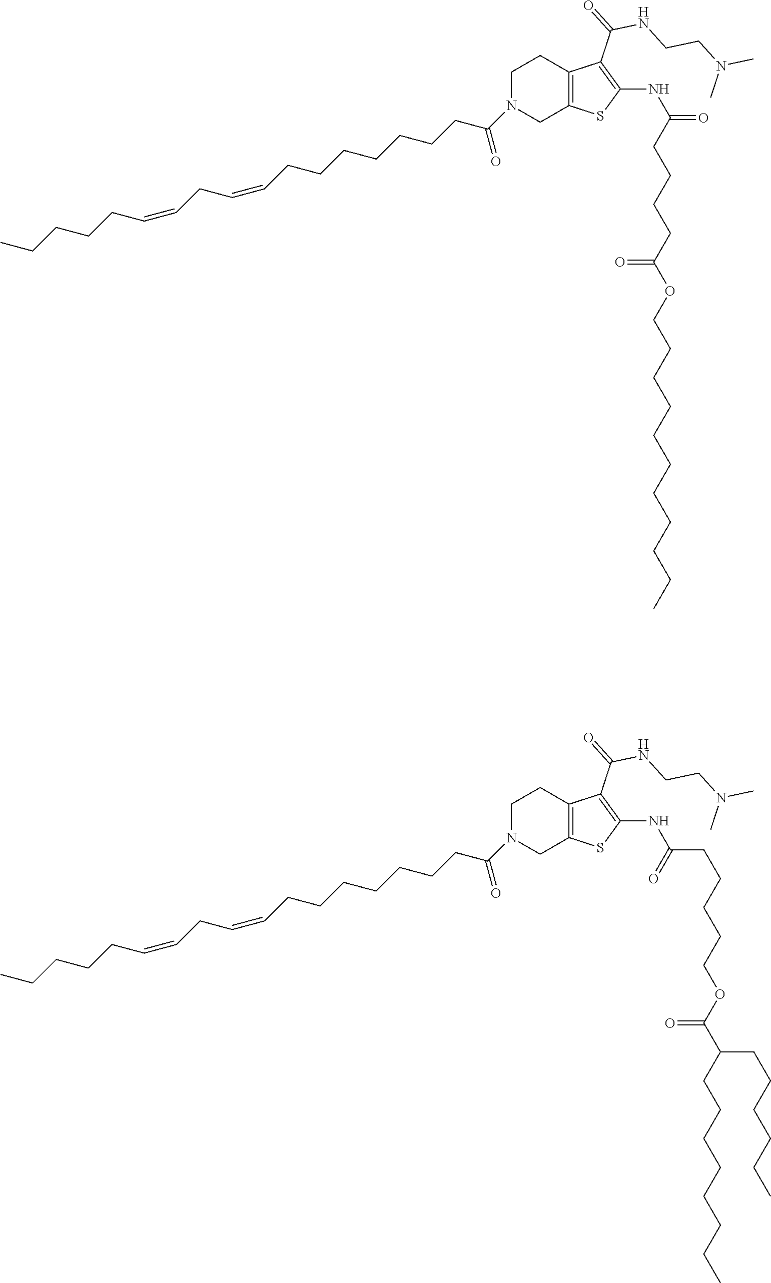

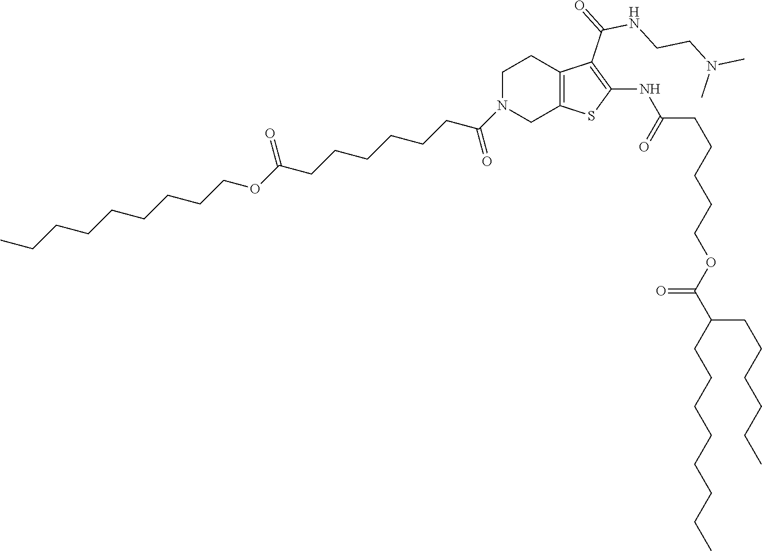 [SMILES: CCCCCCCCCOC(=O)CCCCCCC(=O)N1CCc2c(sc(NC(=O)CCCCCOC(=O)C(CCCCCC)CCCCCCCC)c2C(=O)NCCN(C)C)C1]

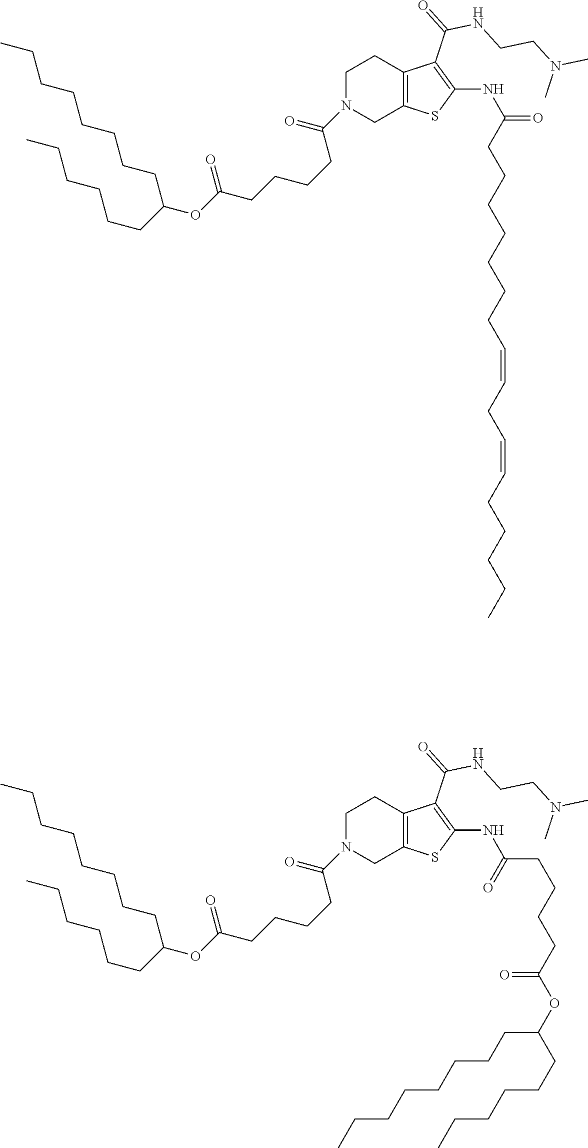

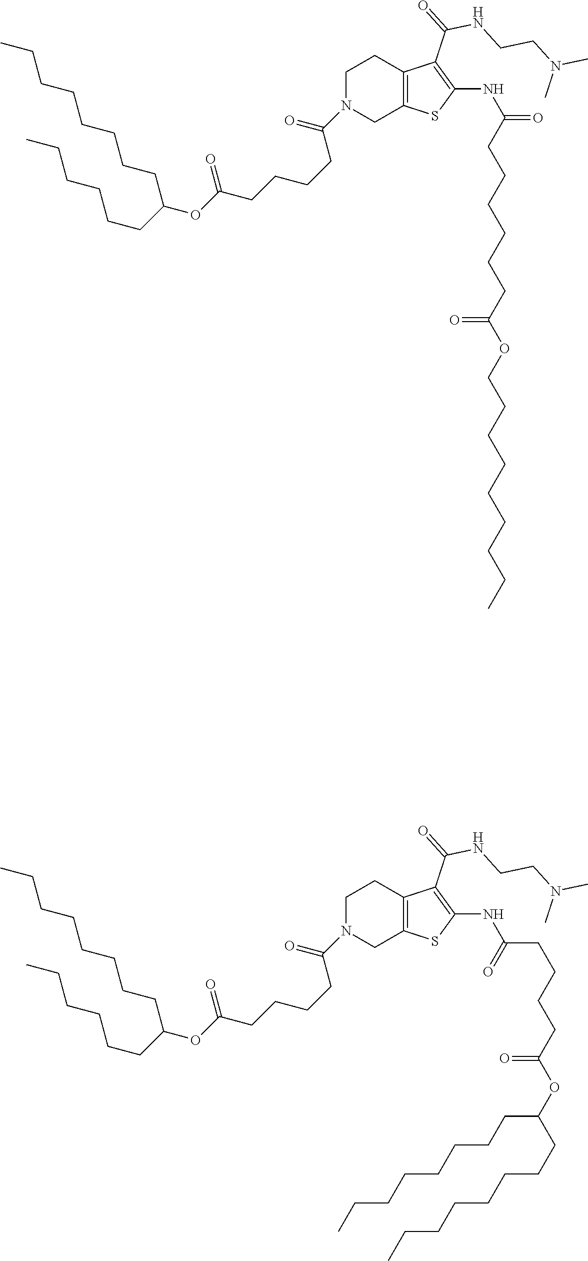

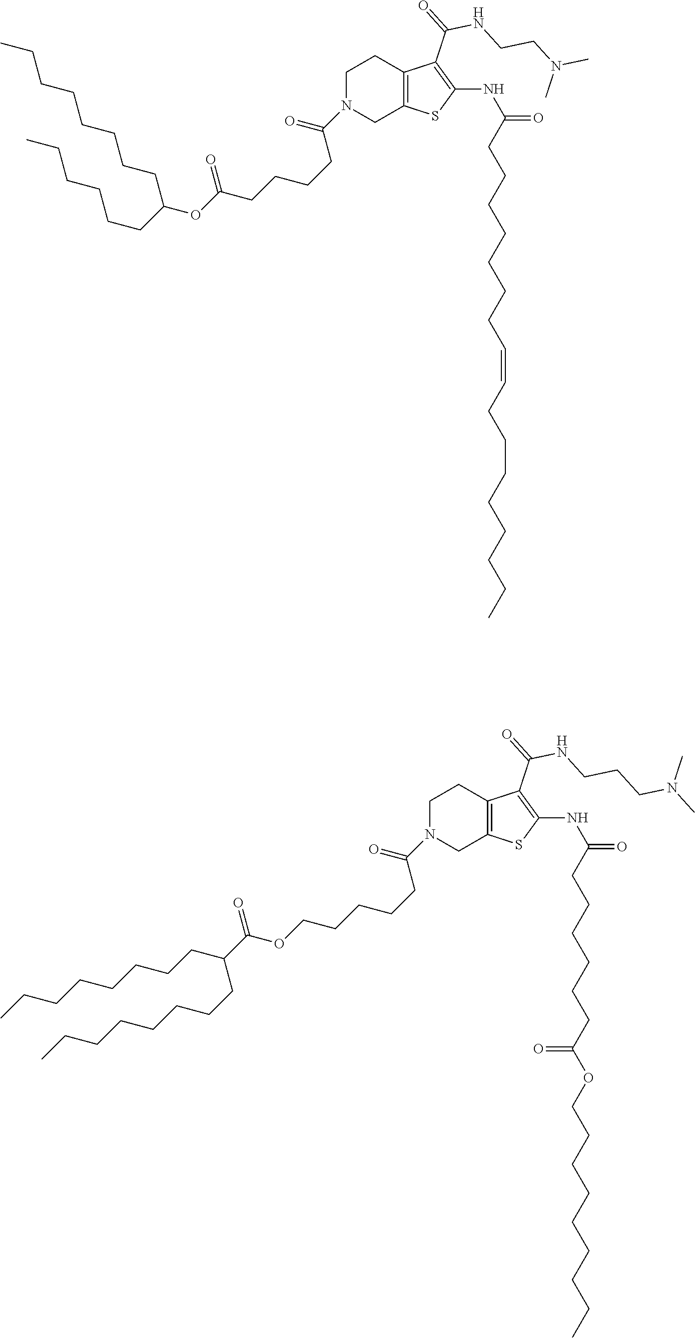

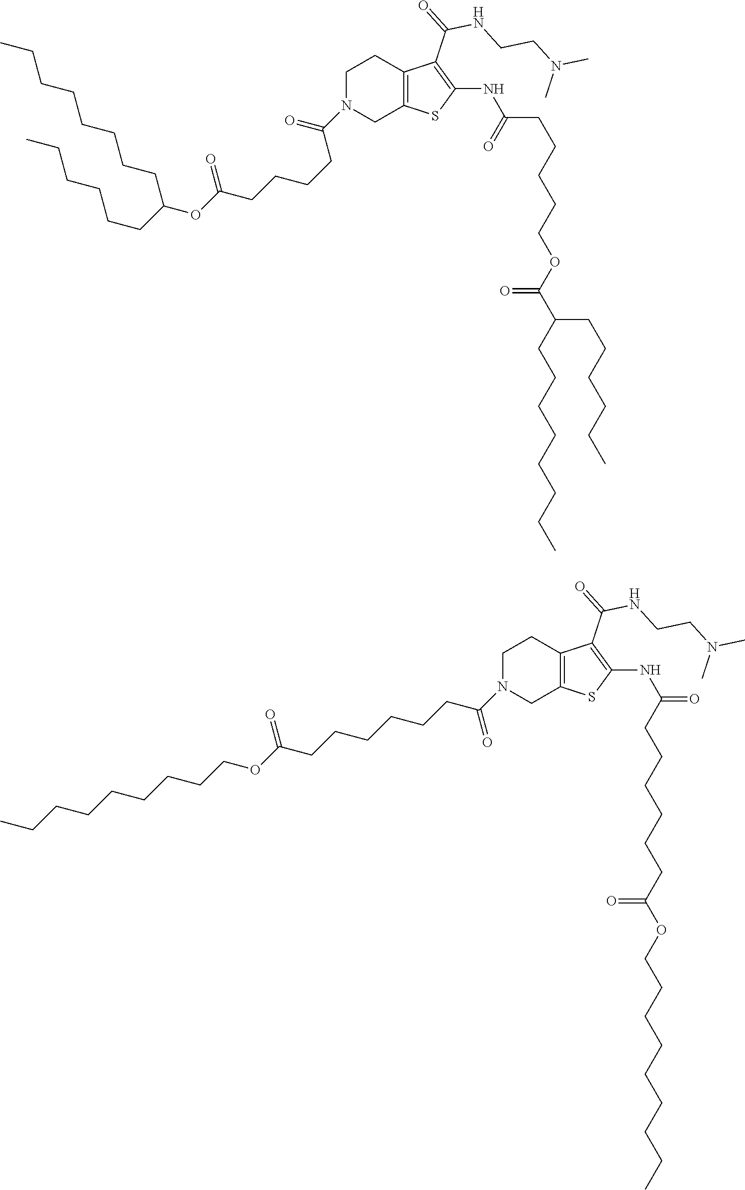

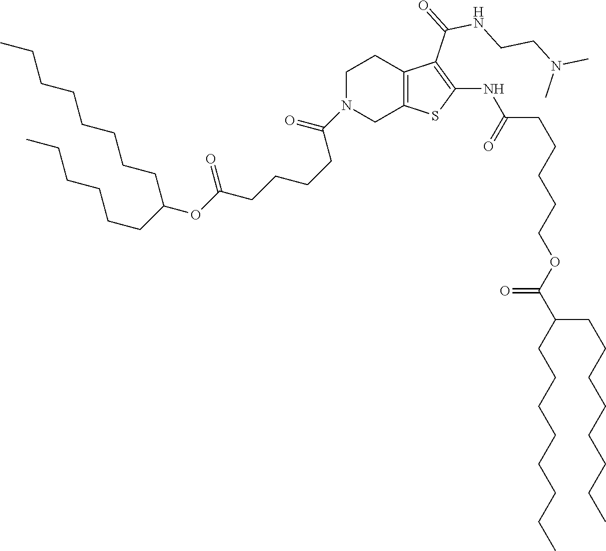 [SMILES: CCCCCCCCC(CCCCCC)OC(=O)CCCCC(=O)N1CCc2c(sc(NC(=O)CCCCCOC(=O)C(CCCCCCCC)CCCCCCCC)c2C(=O)NCCN(C)C)C1]

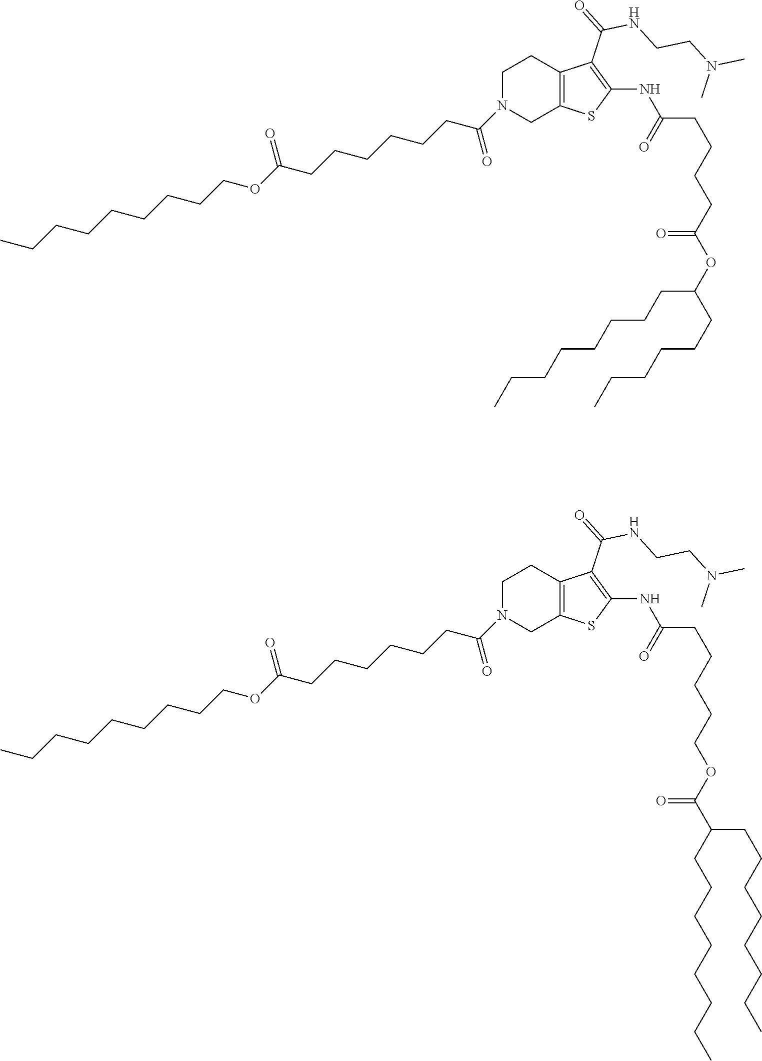

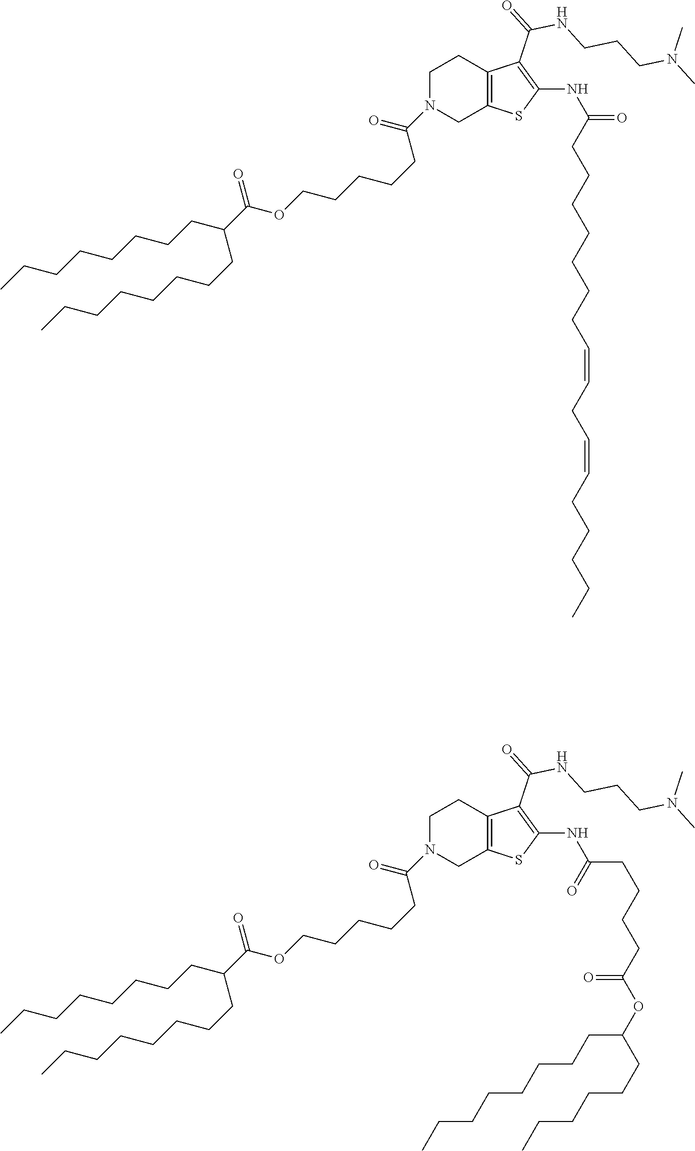

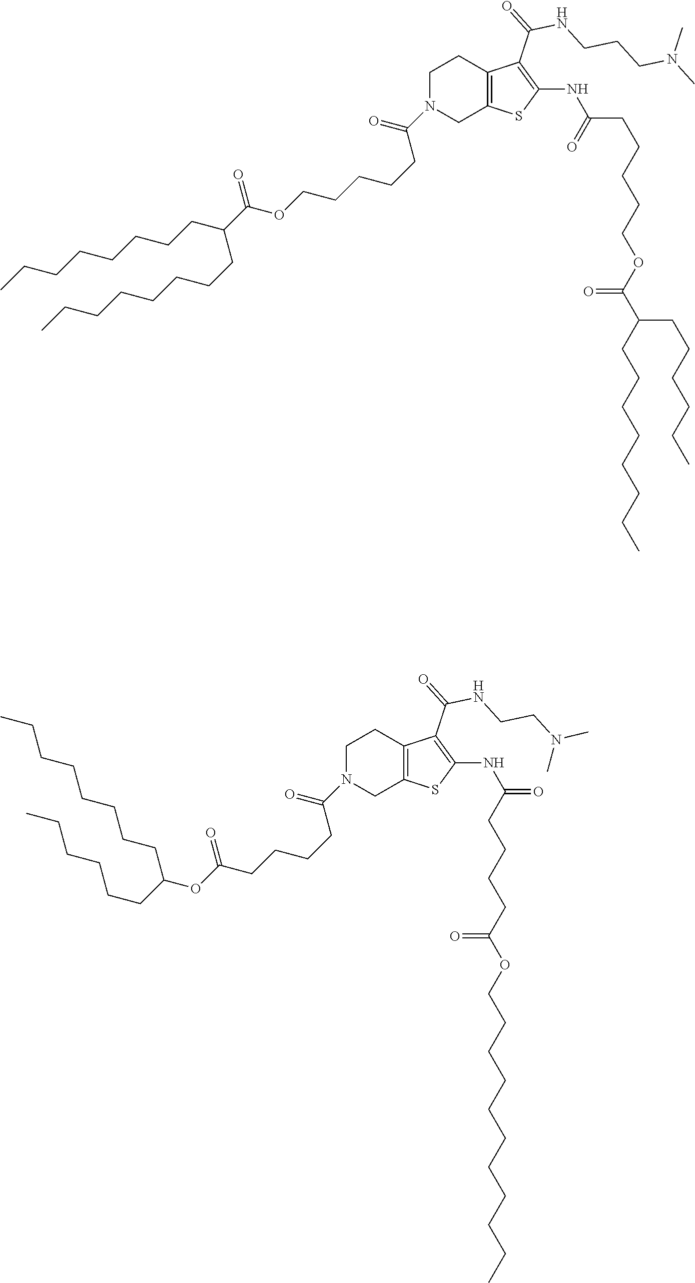

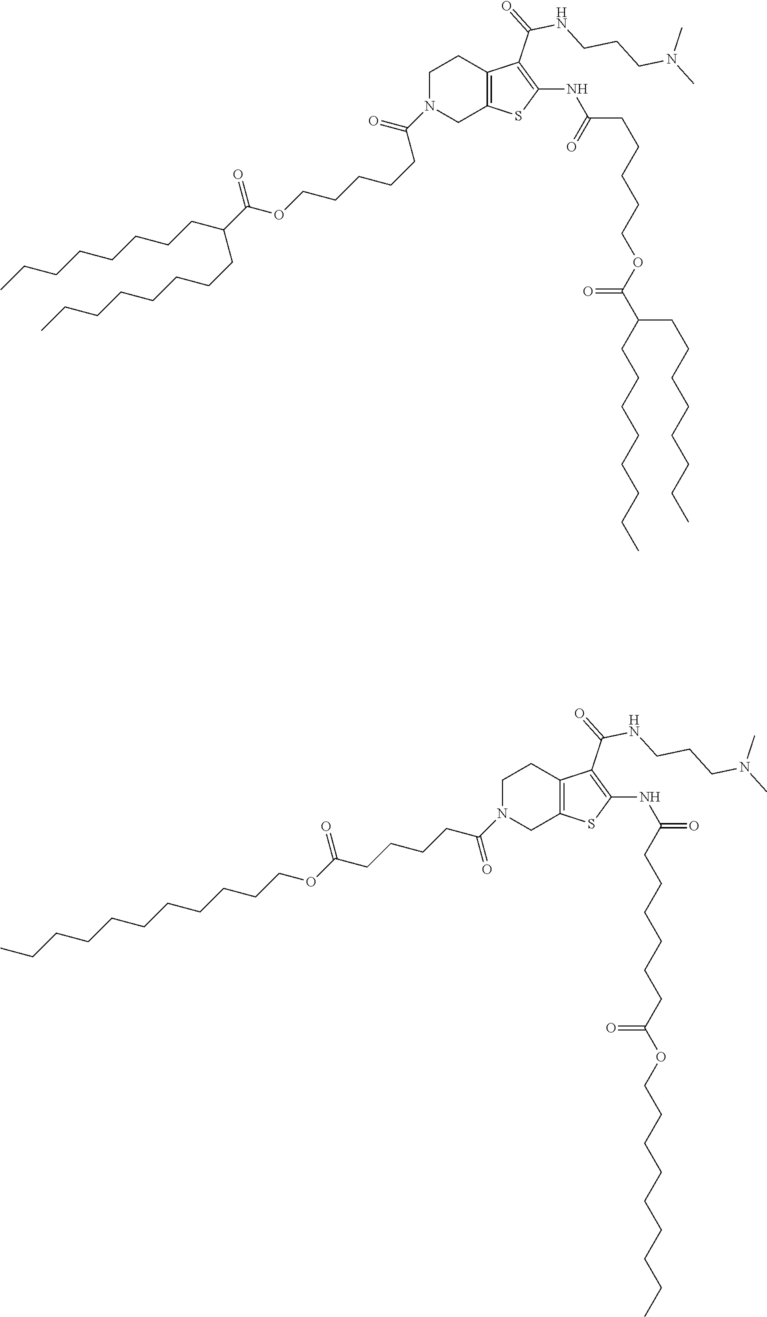

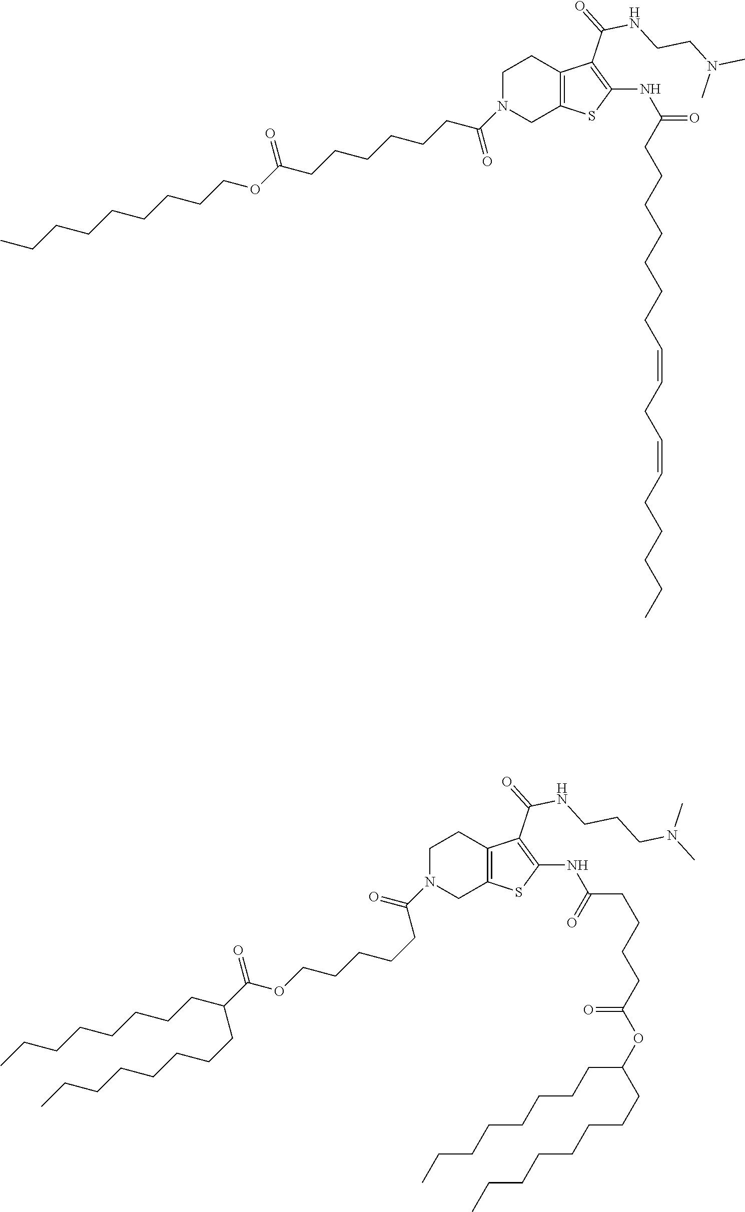

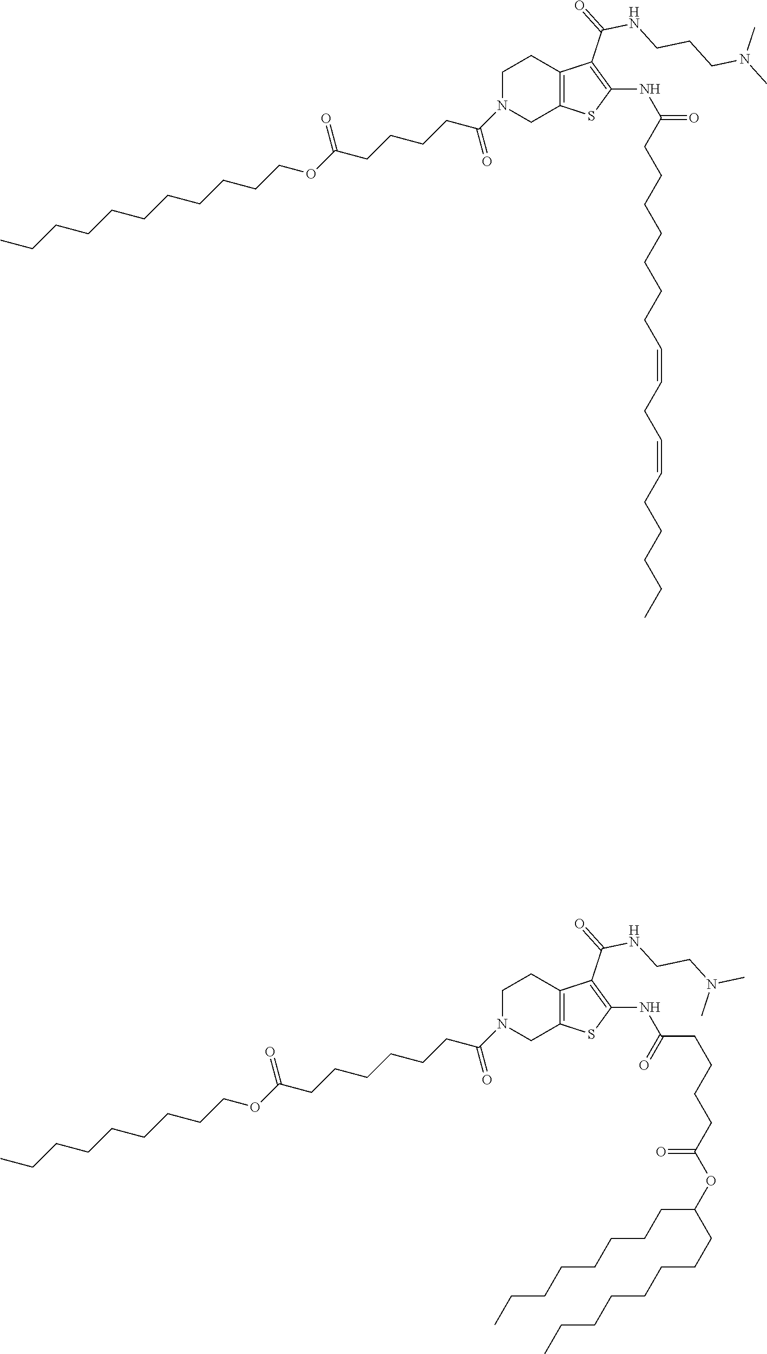

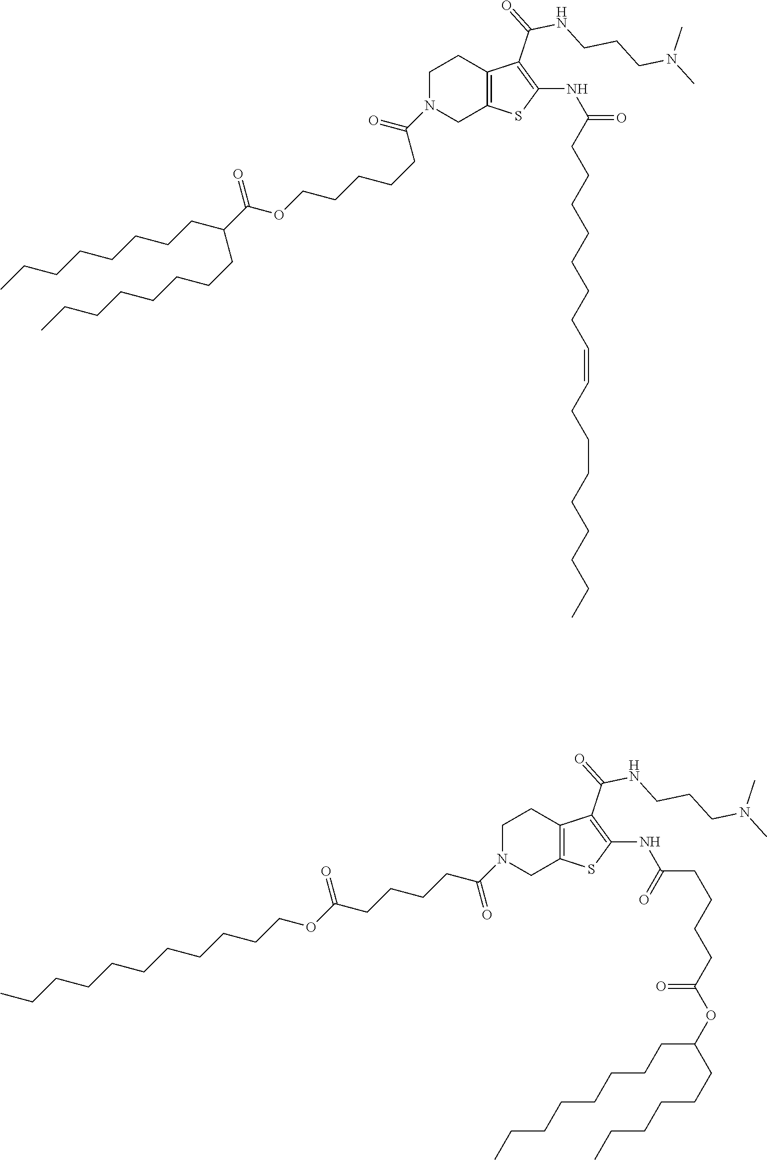

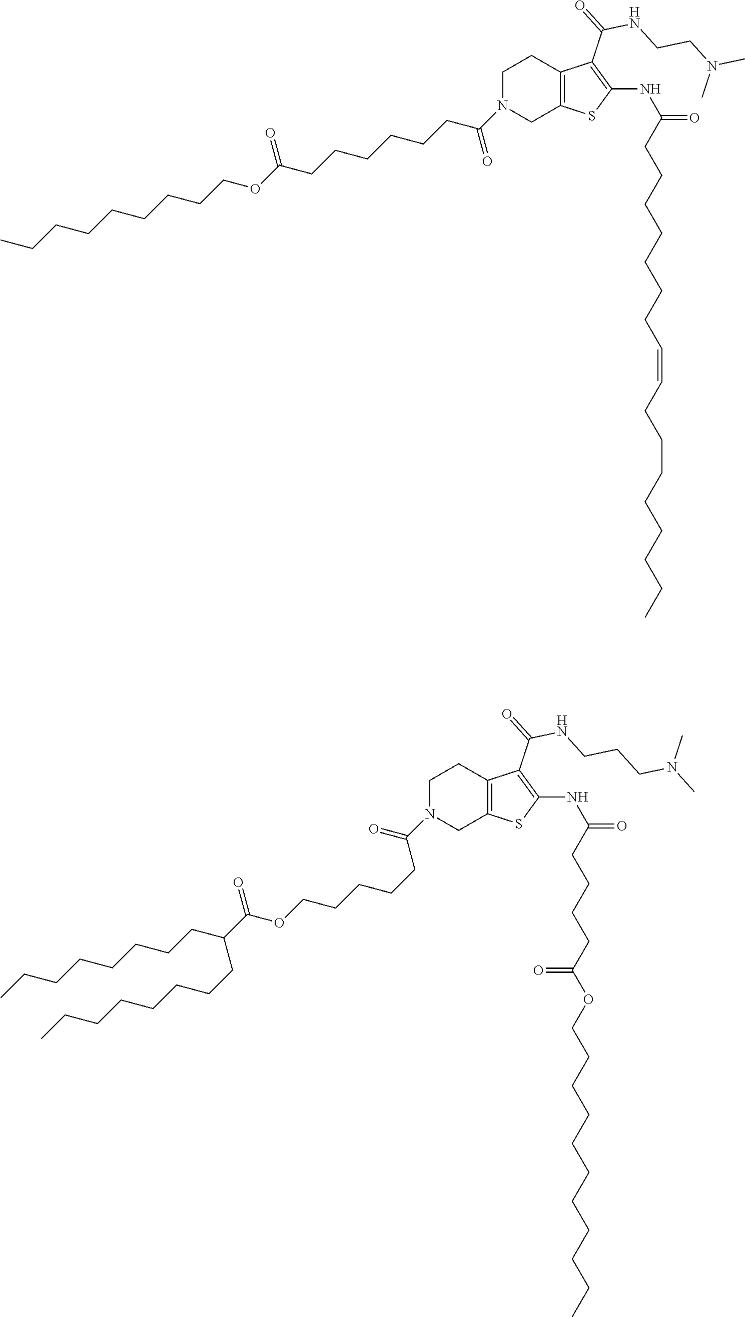

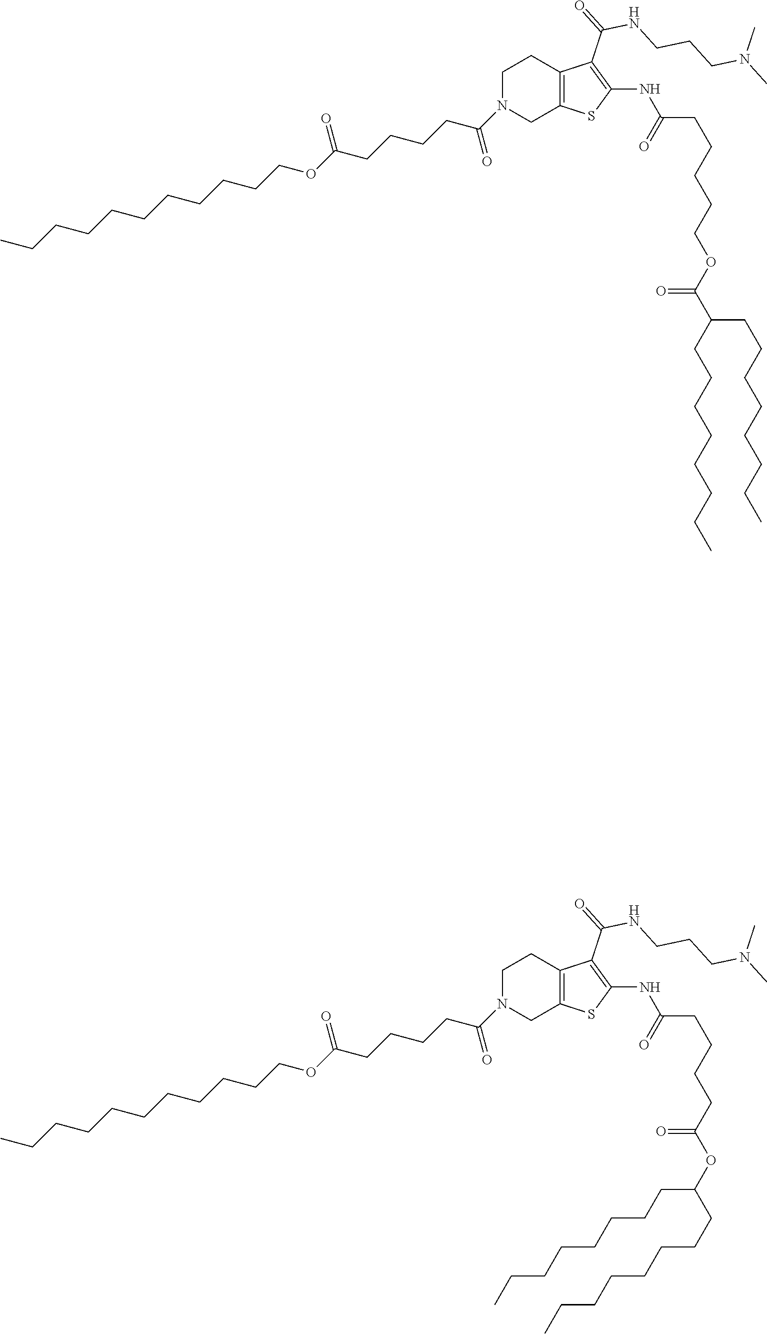

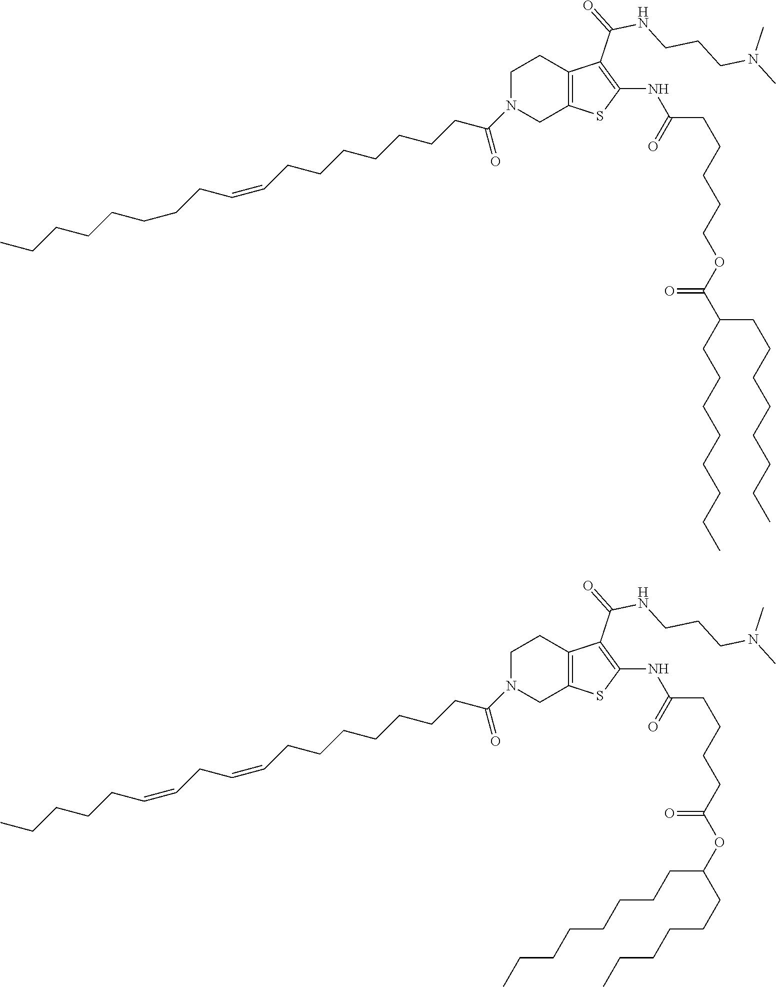

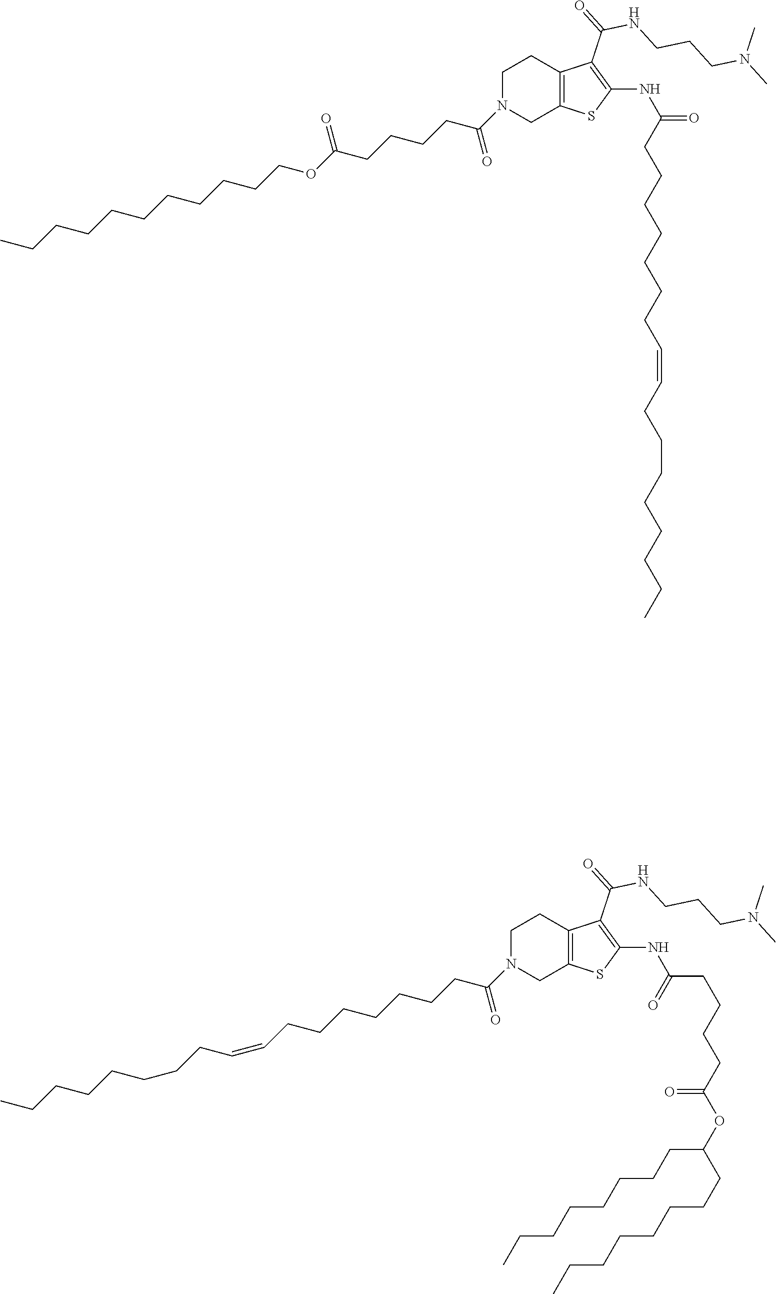

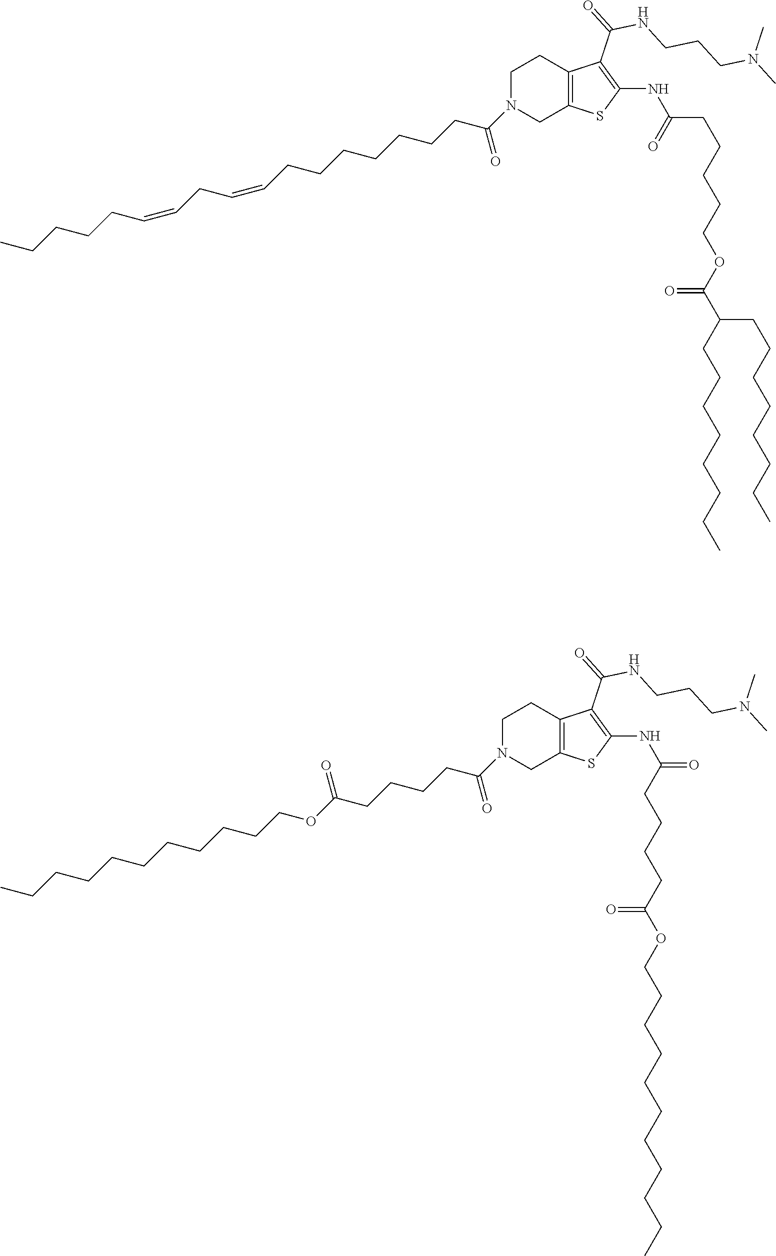

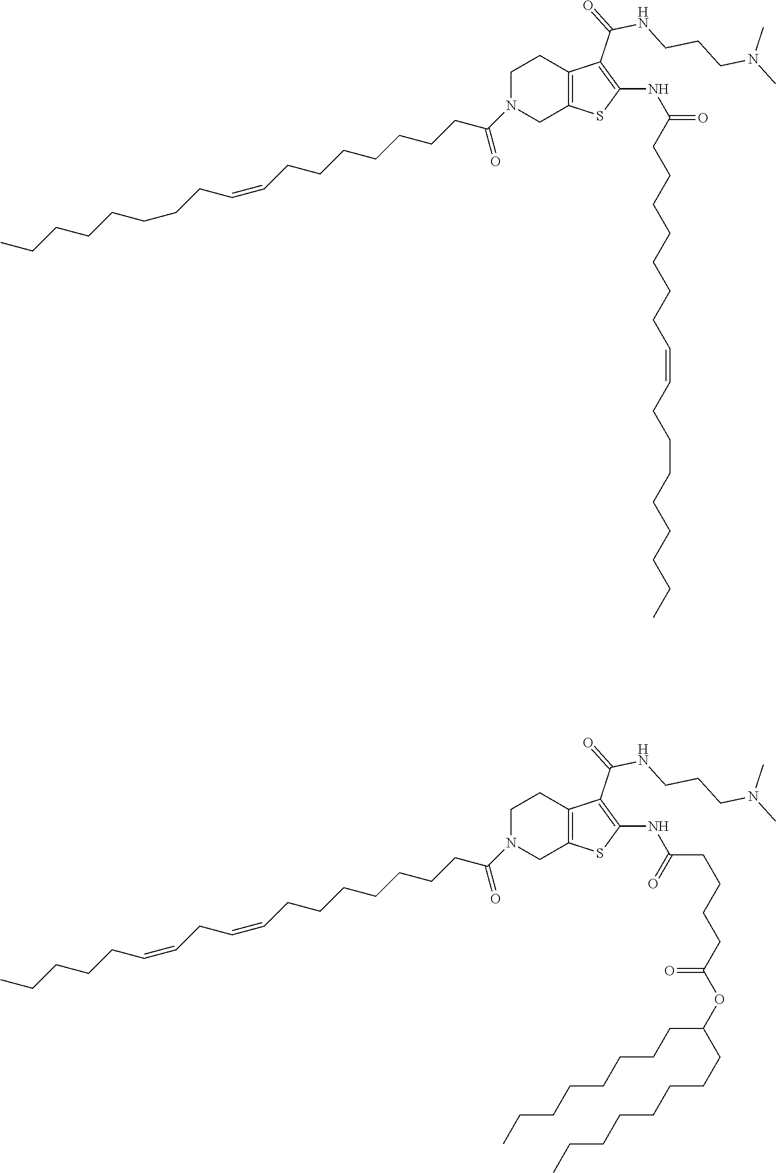

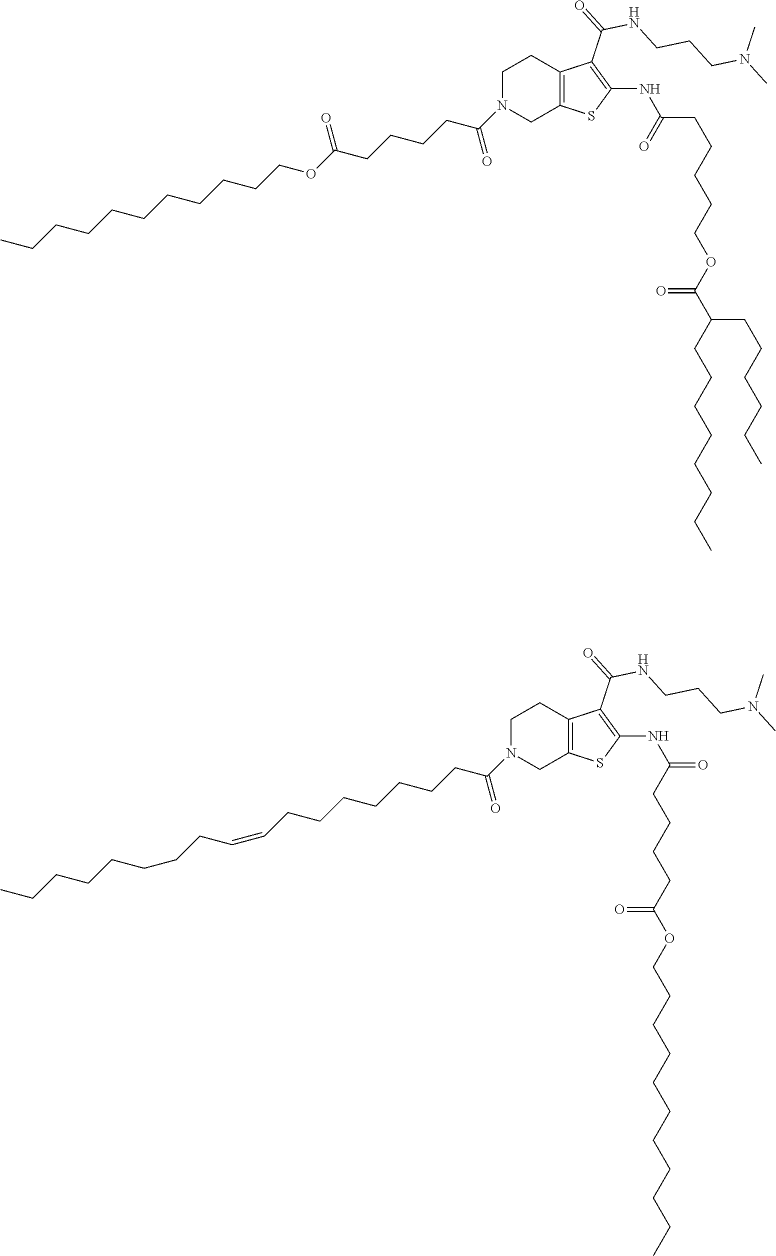

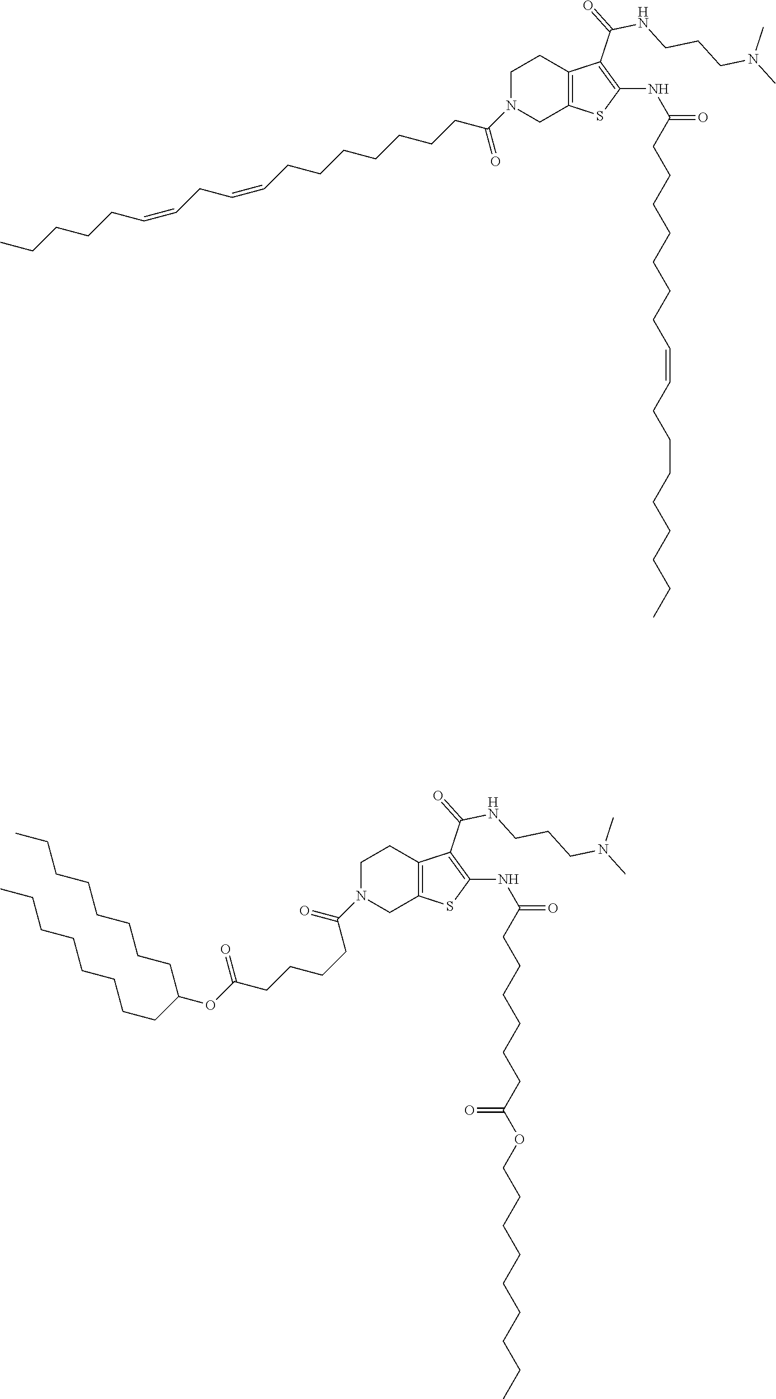

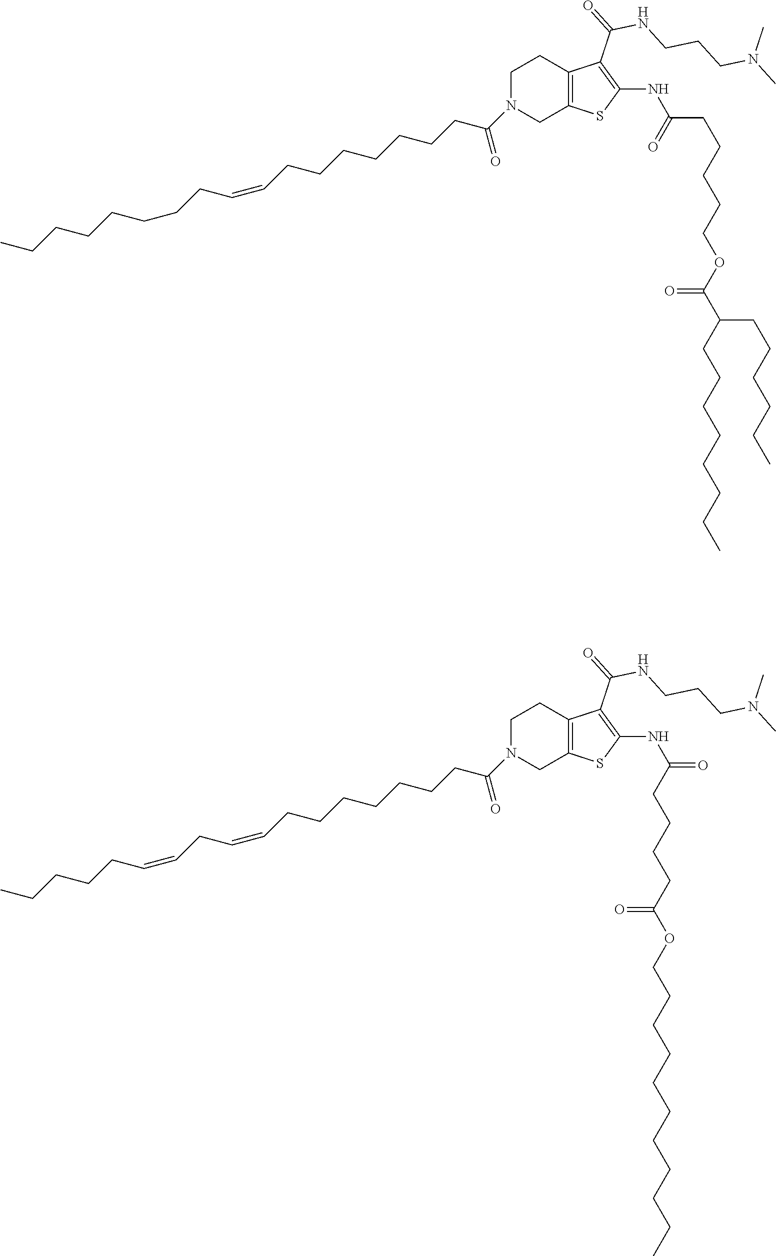

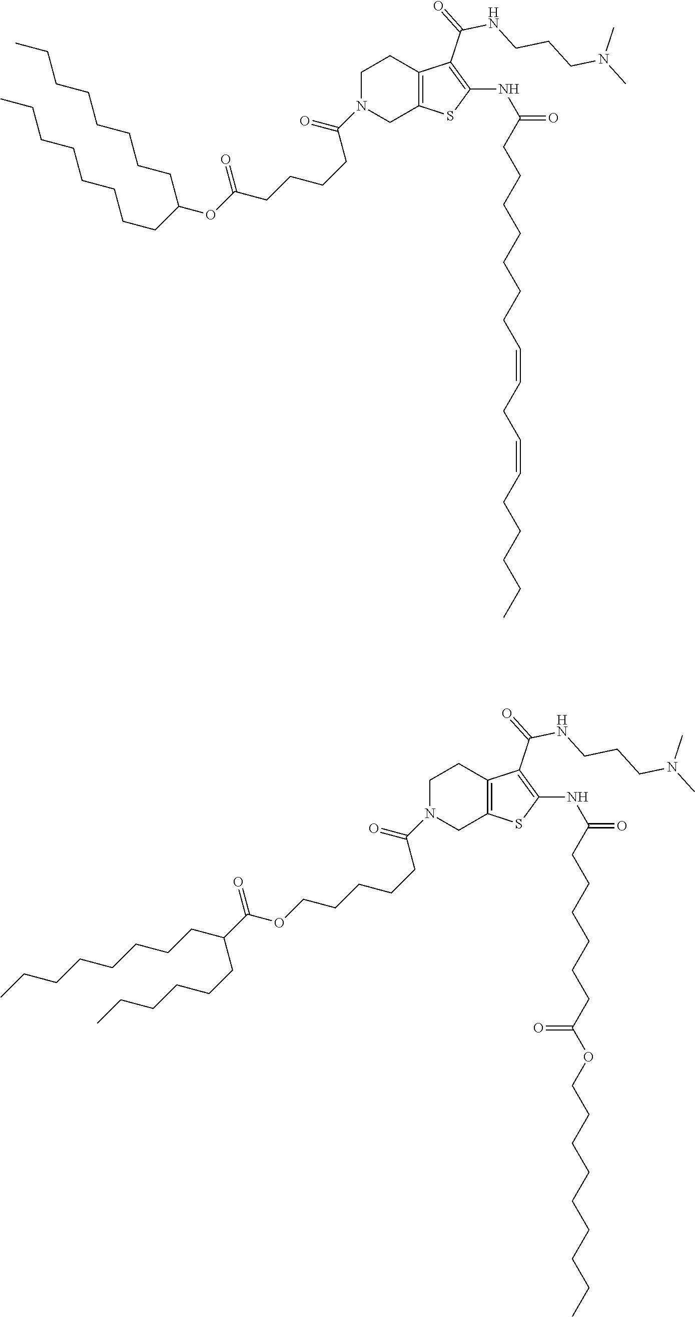

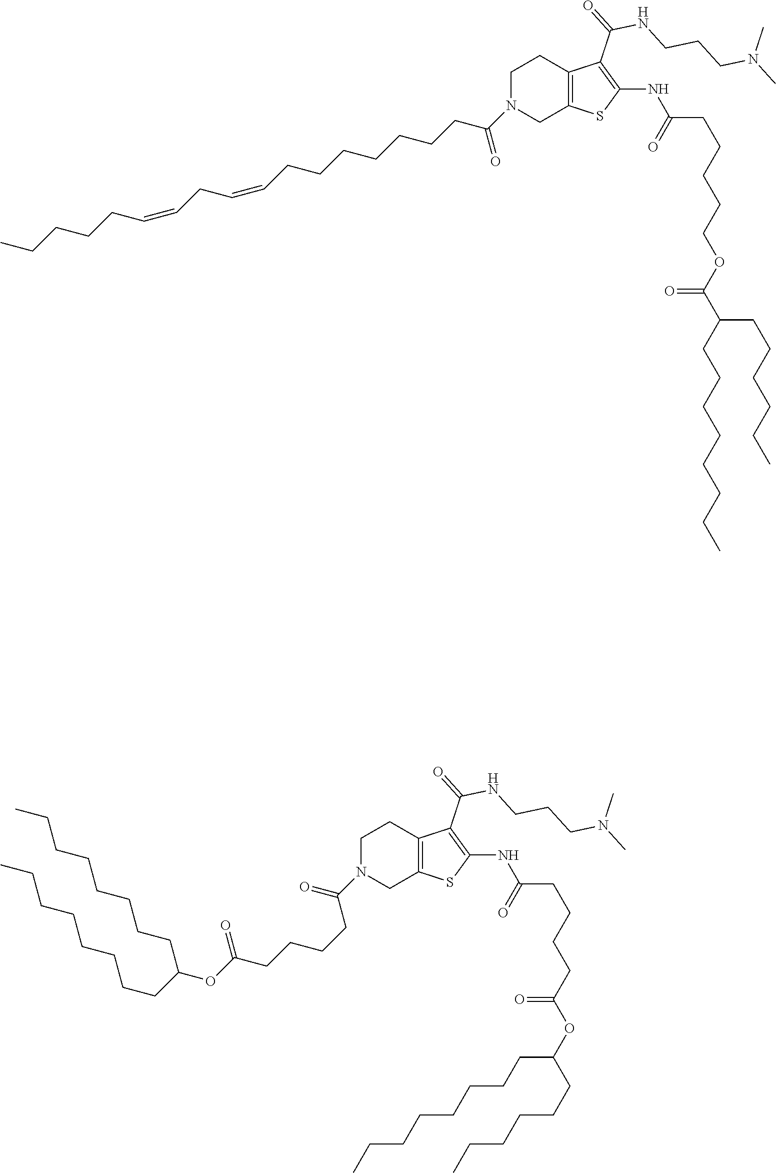

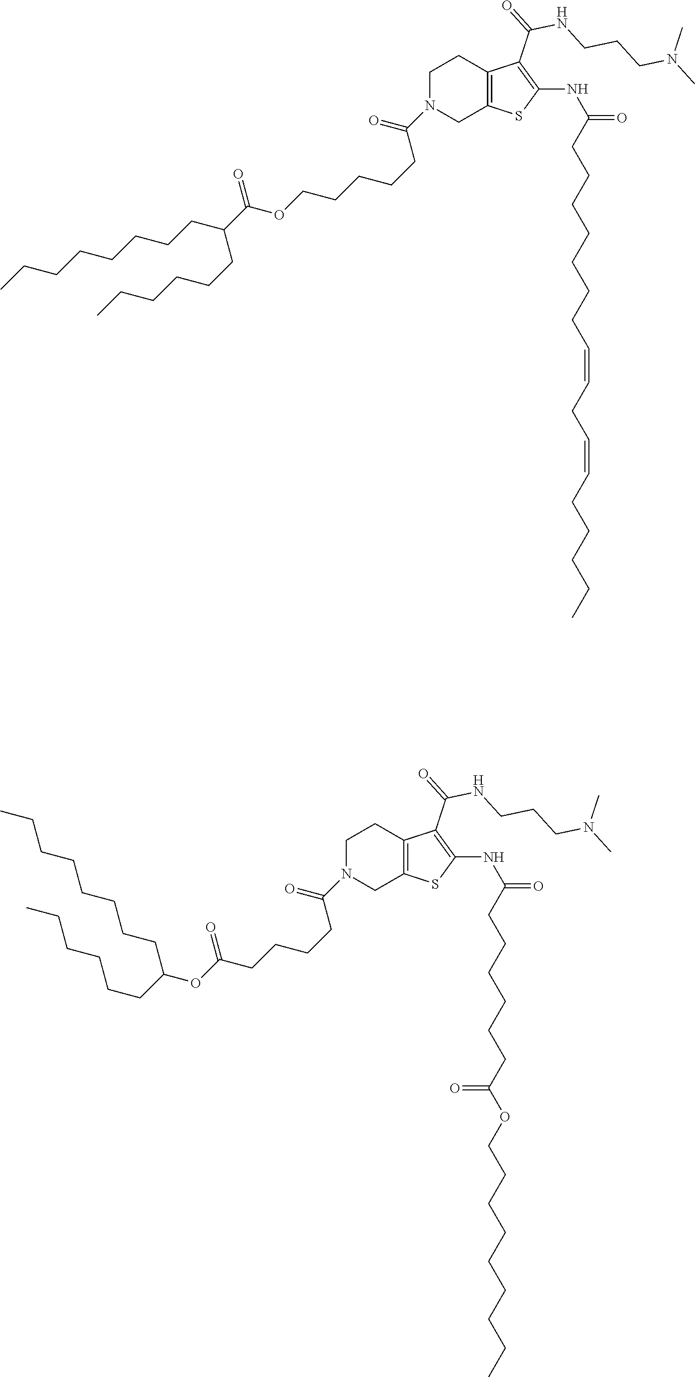

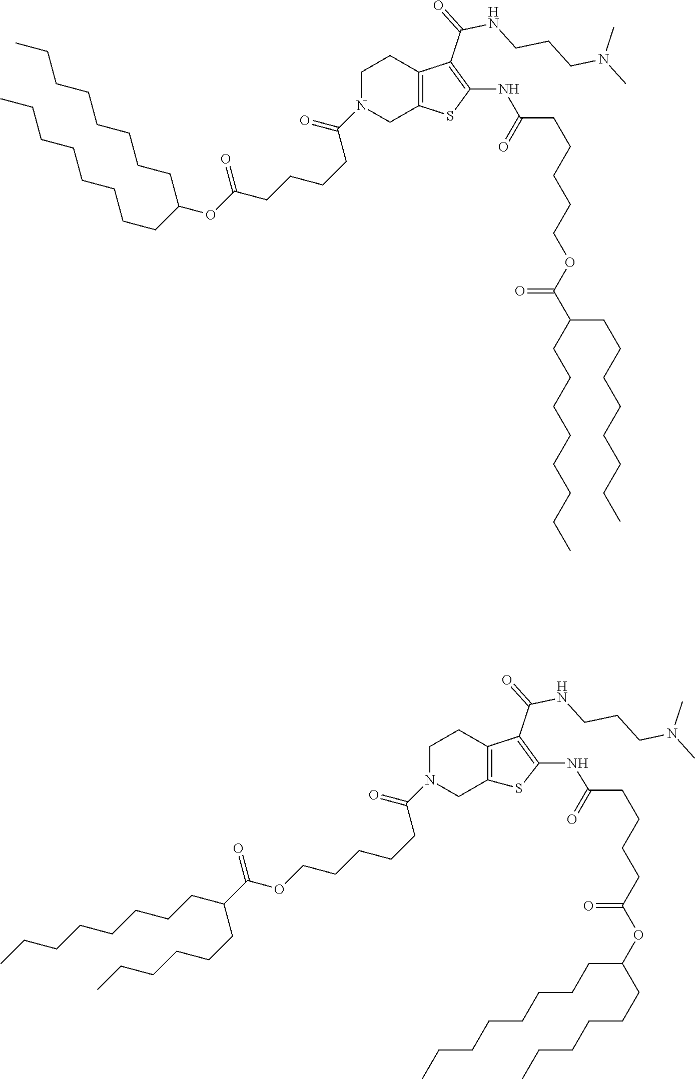

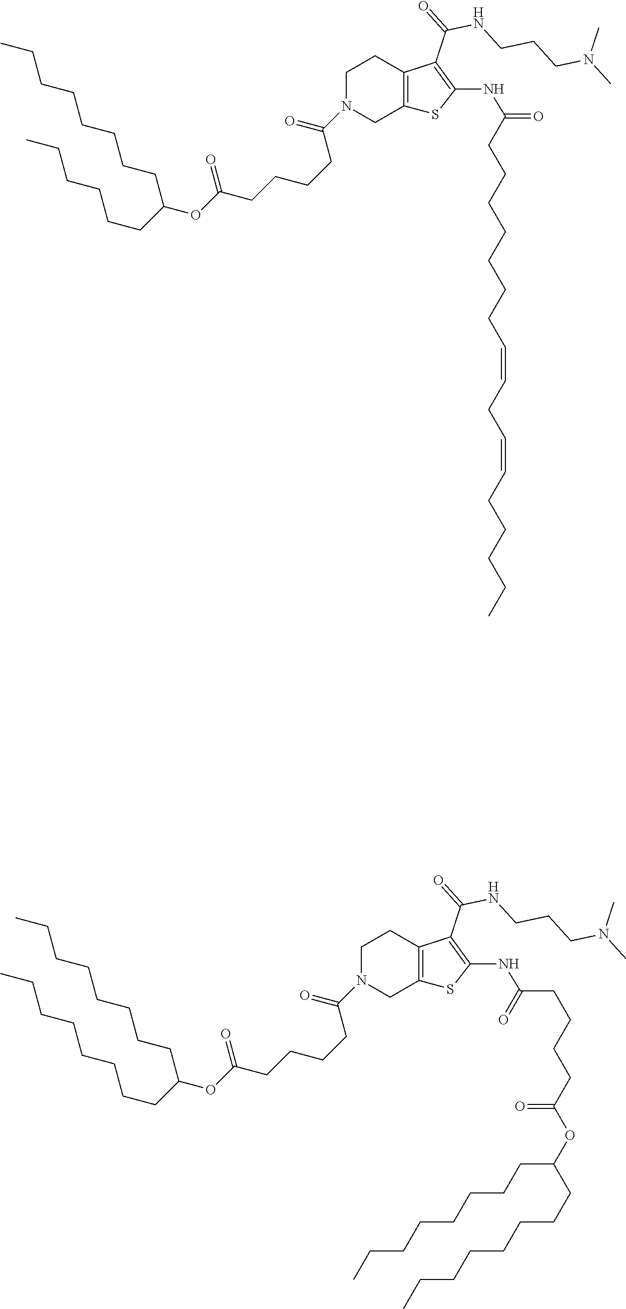

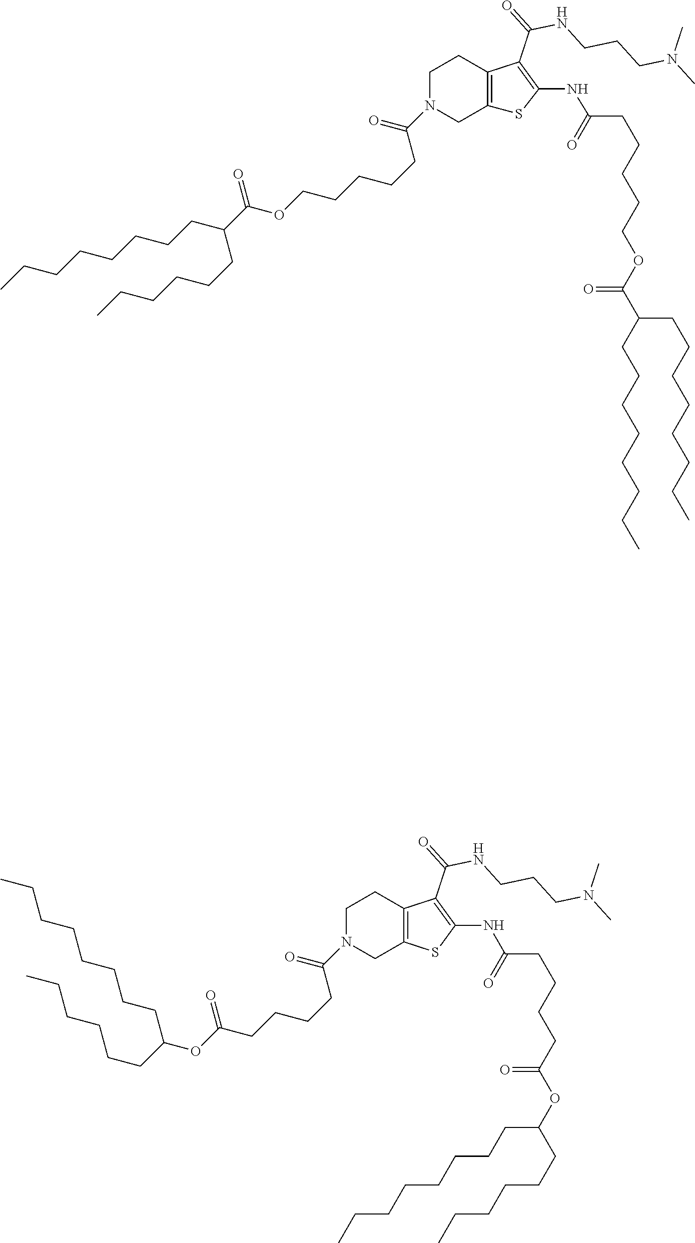

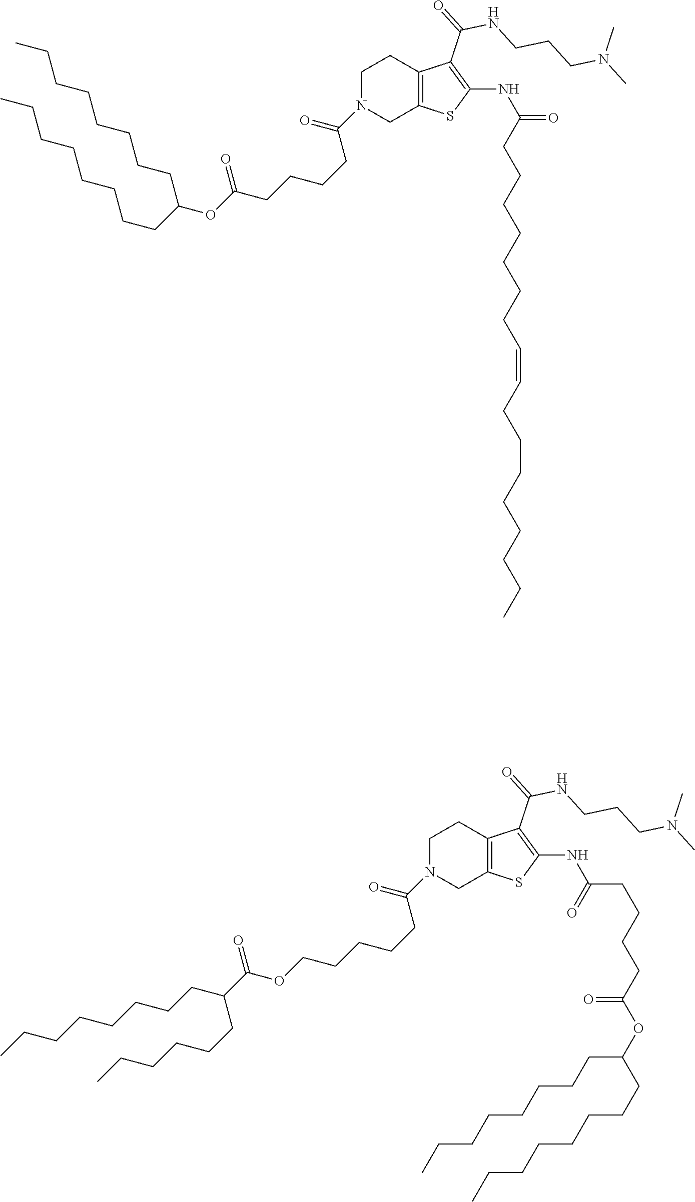

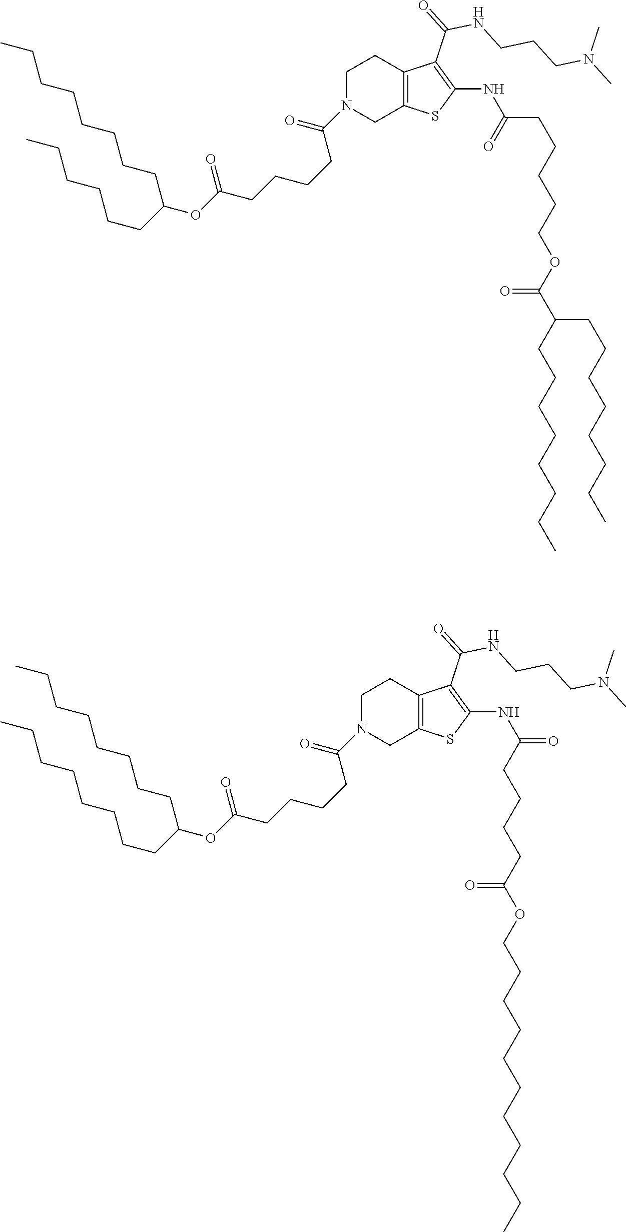

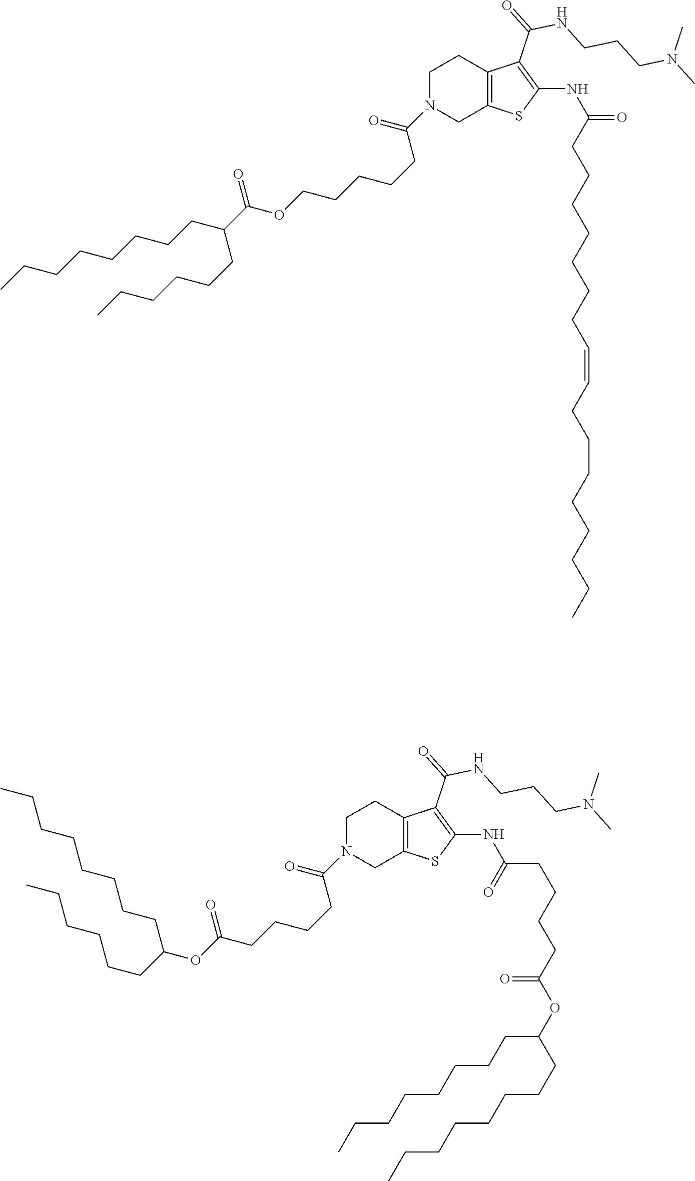

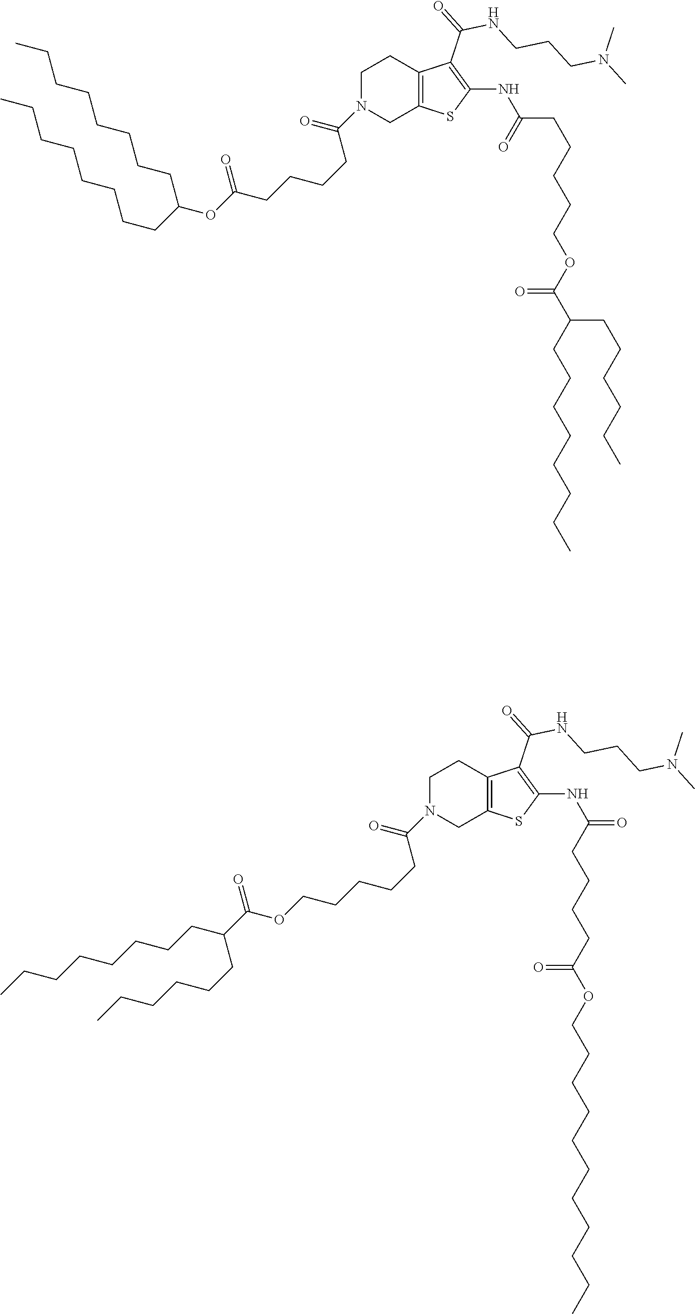

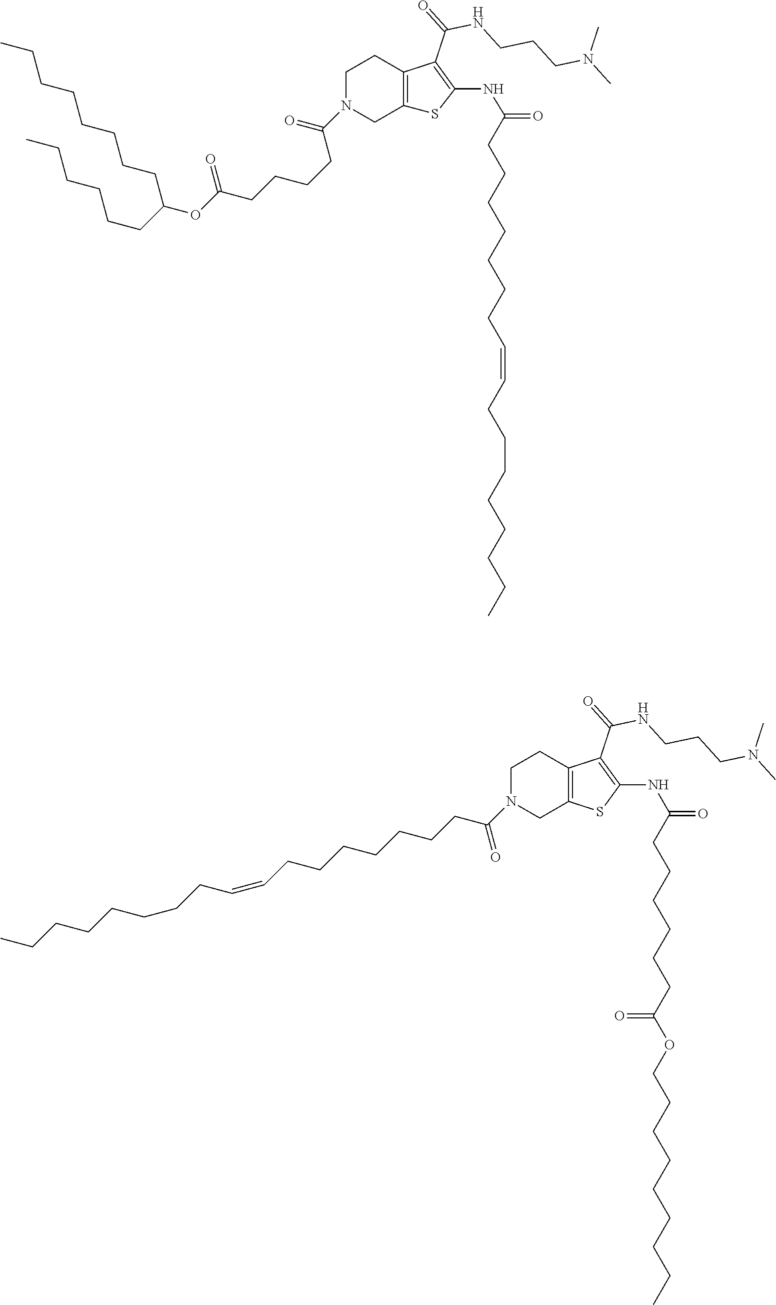

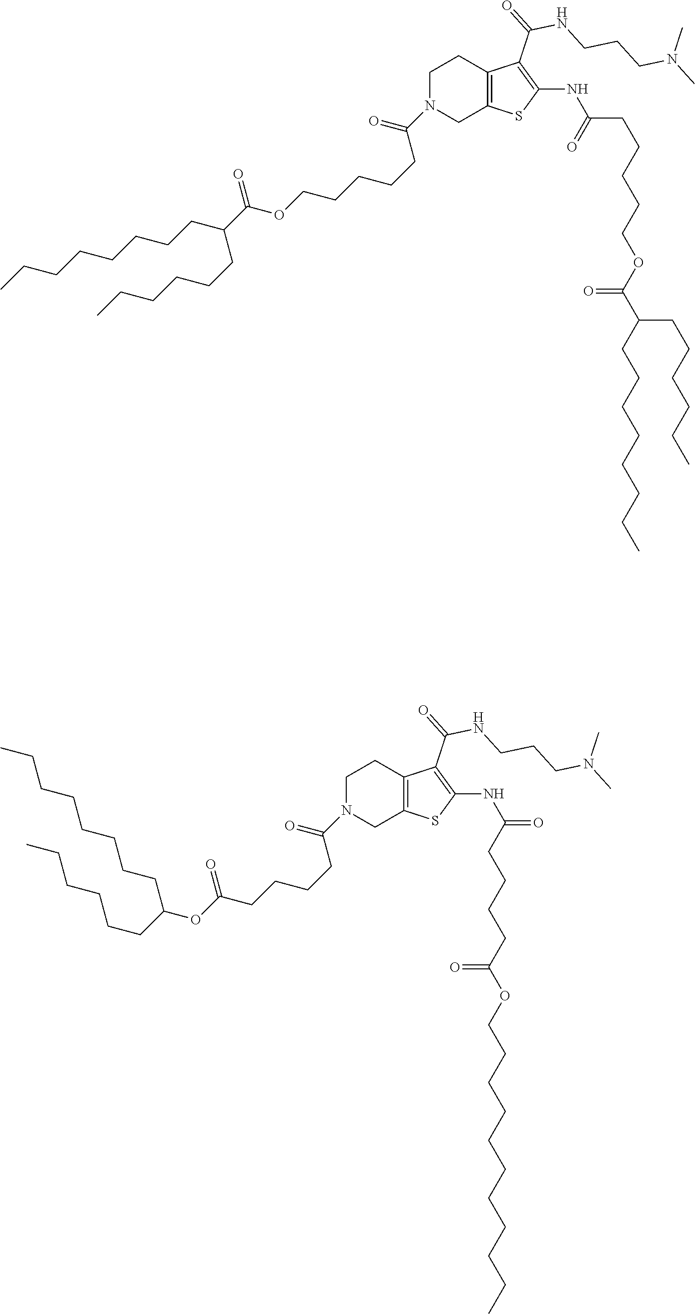

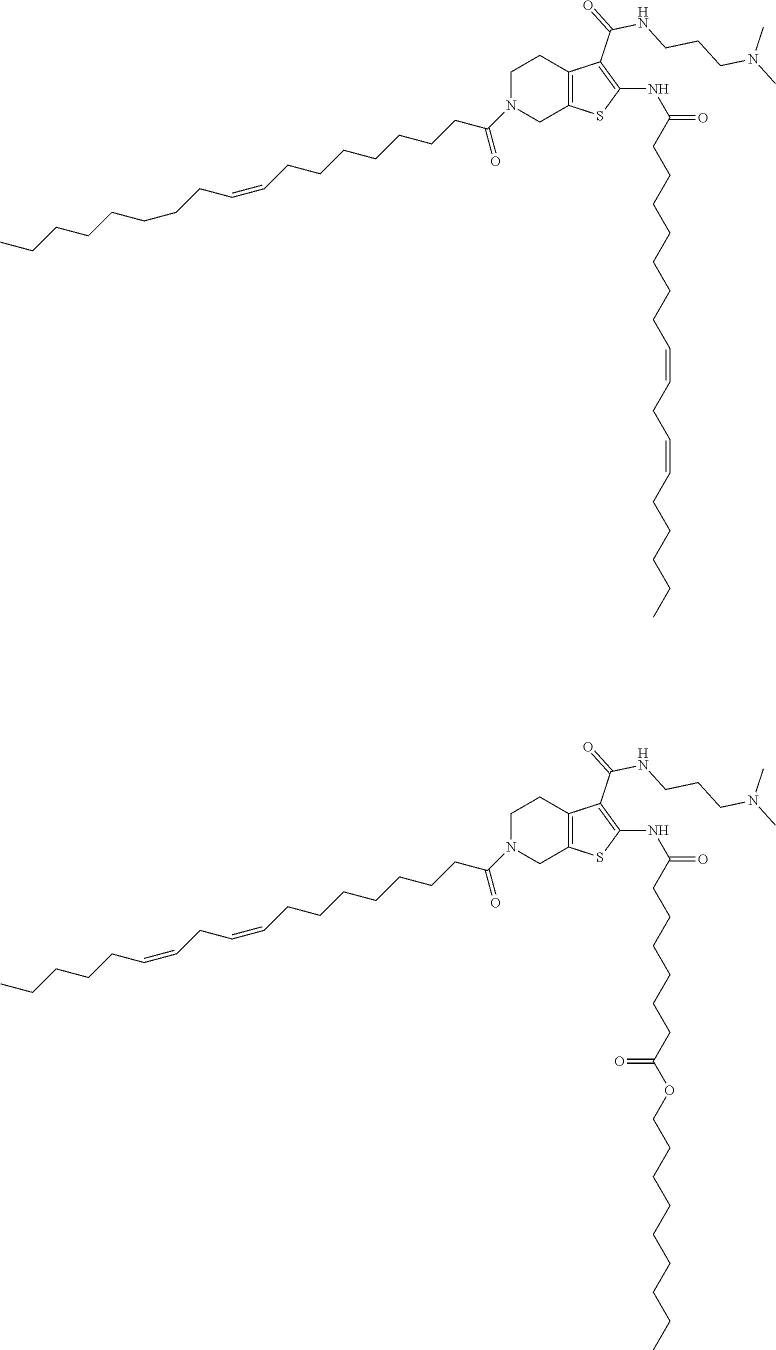

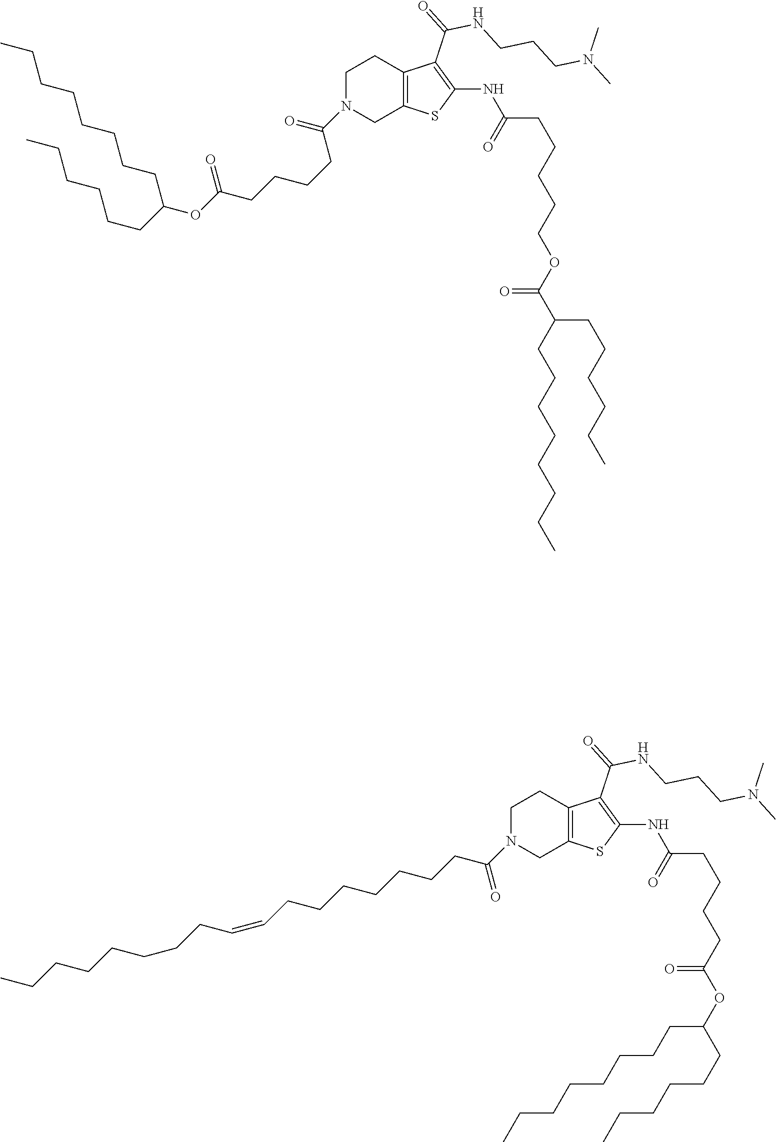

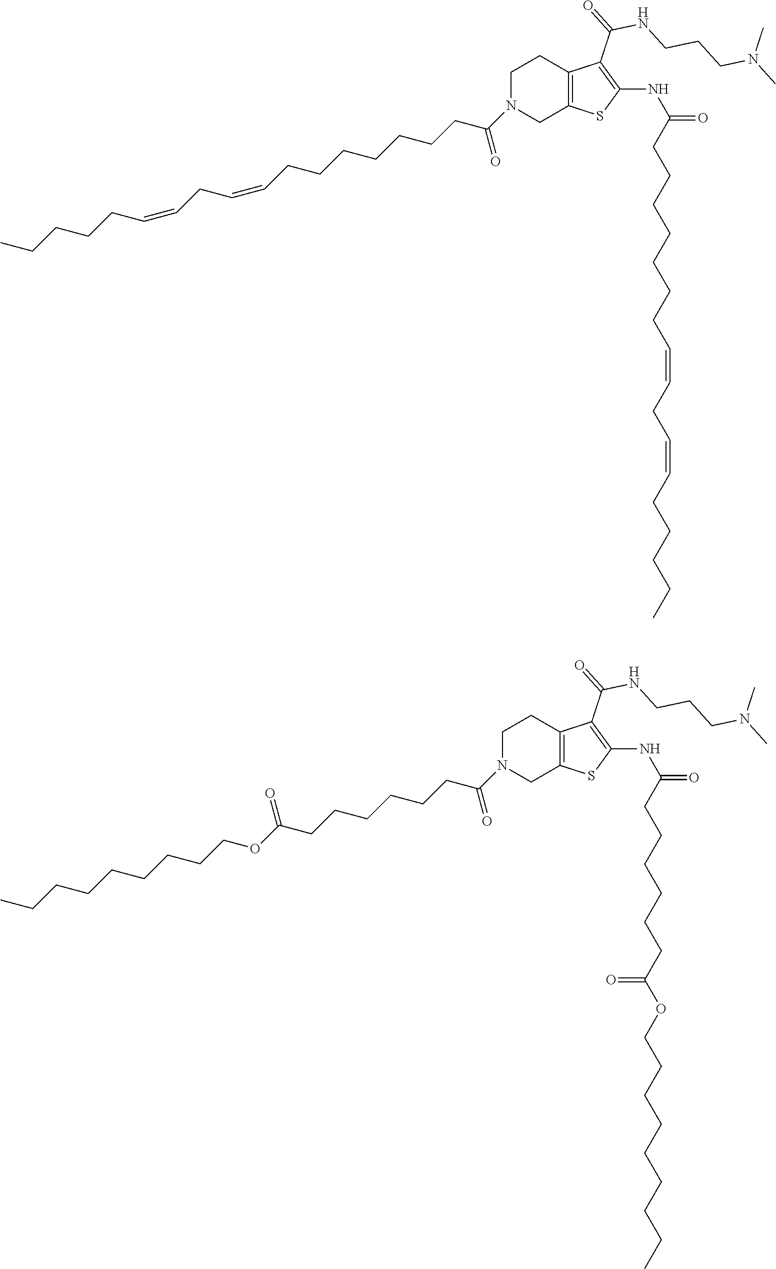

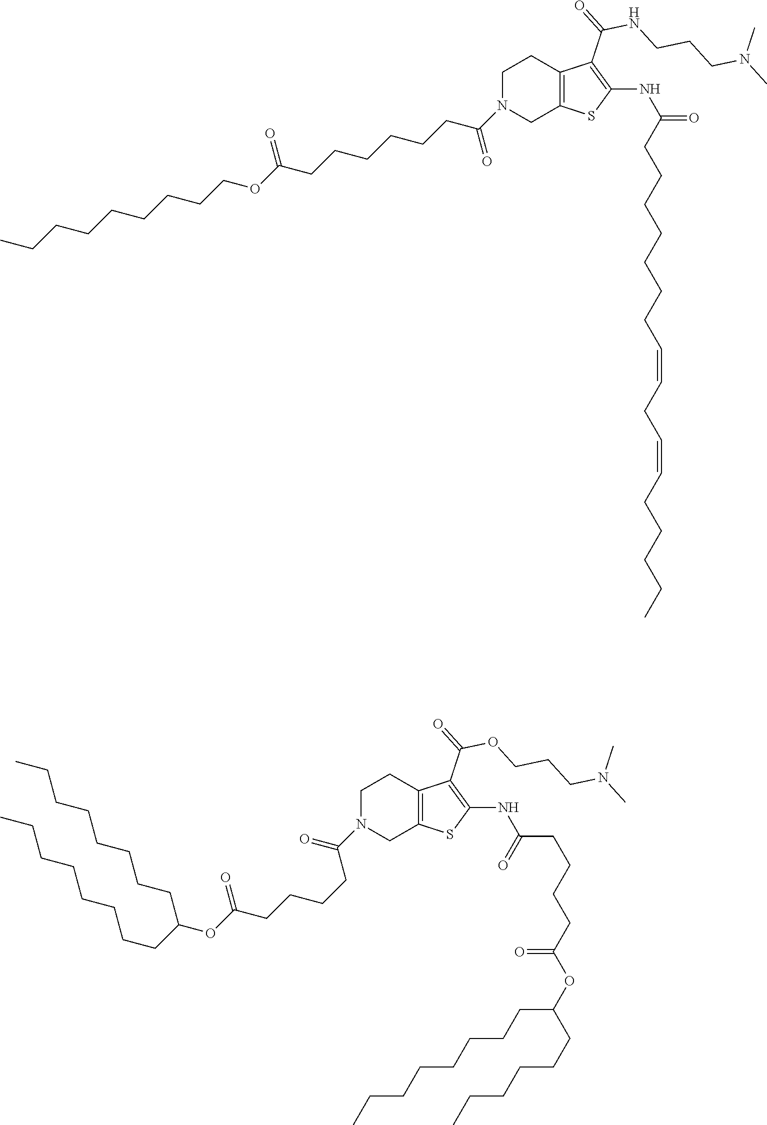

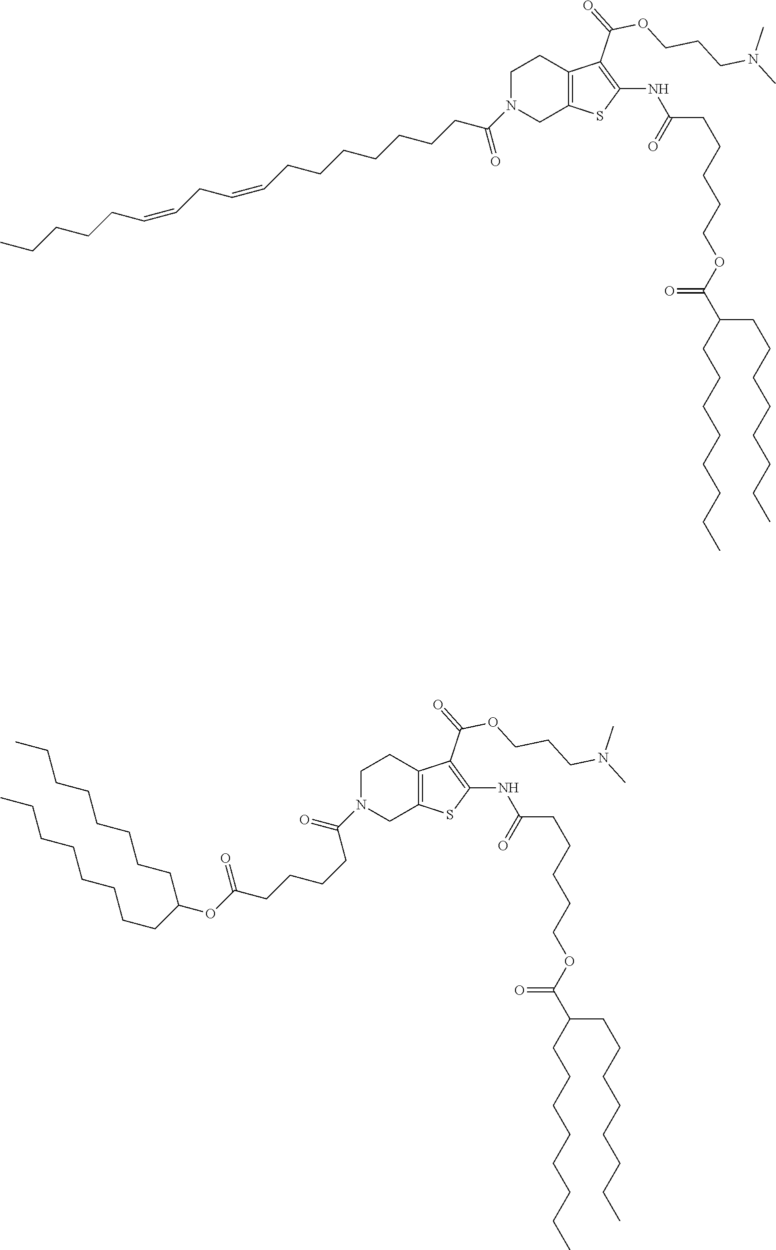

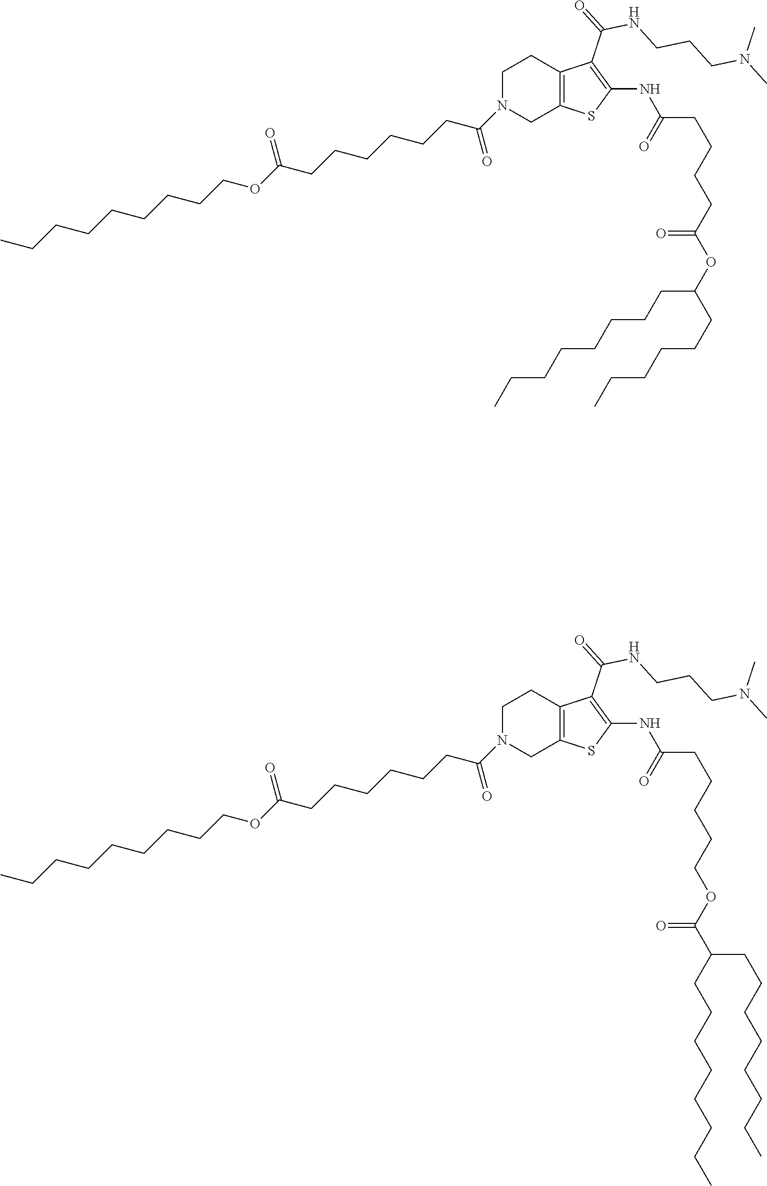

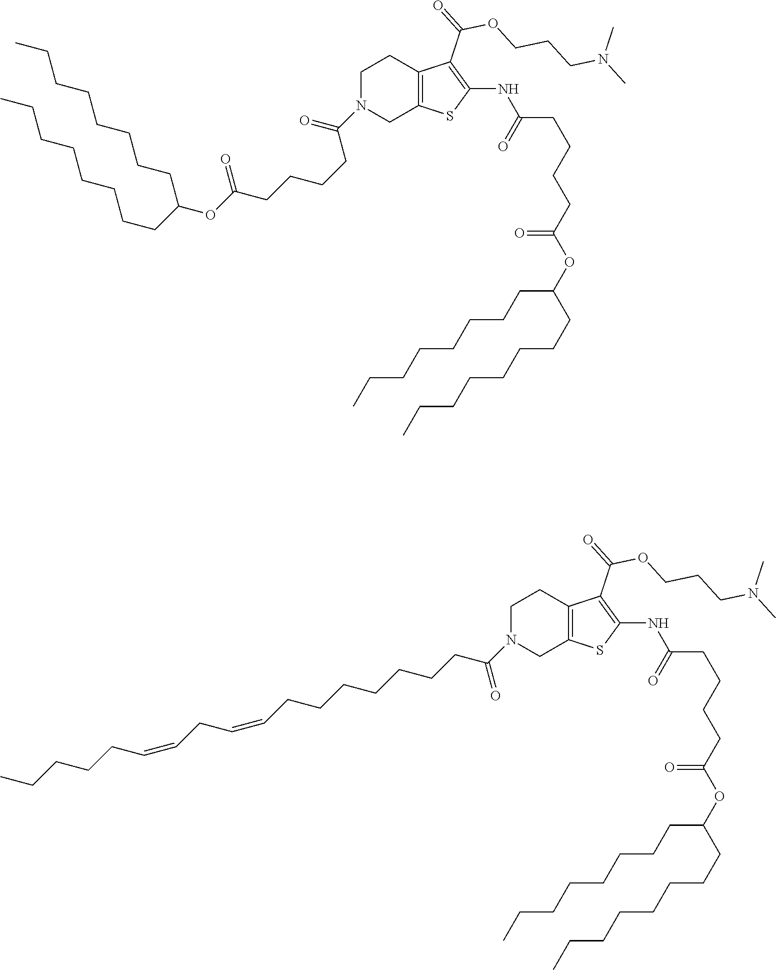

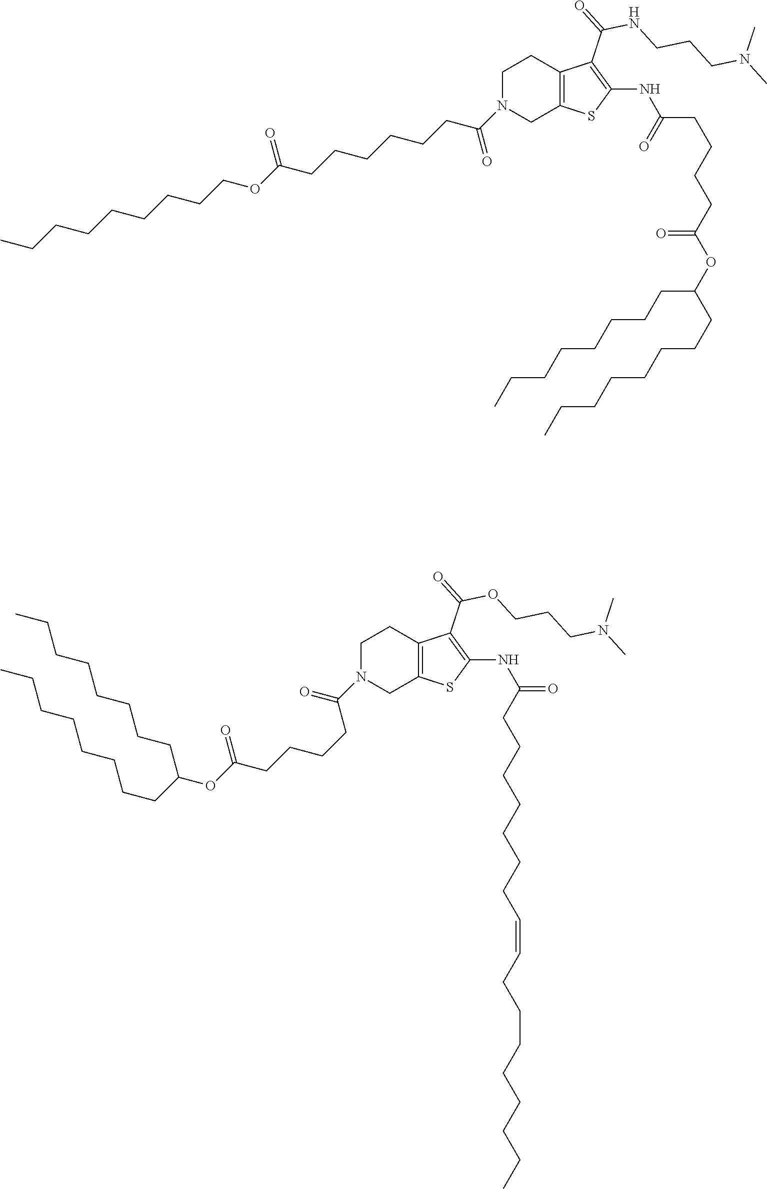

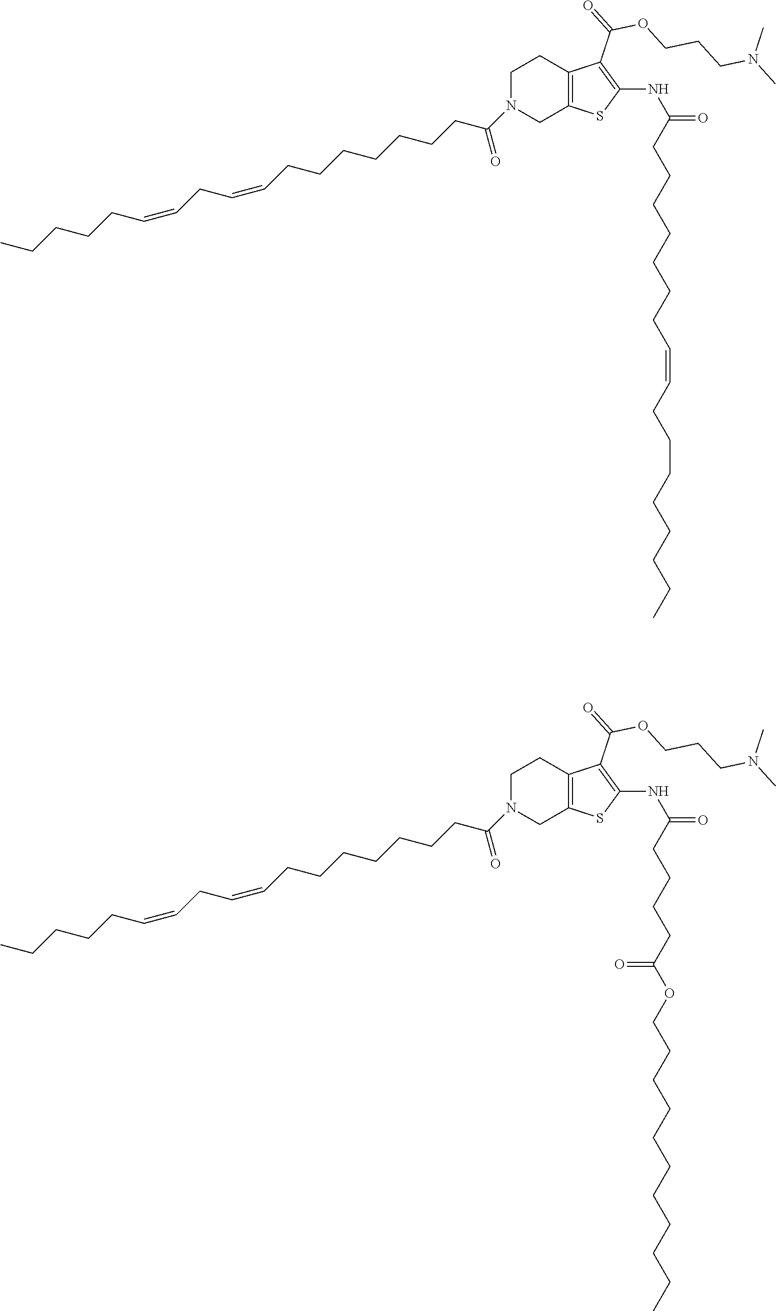

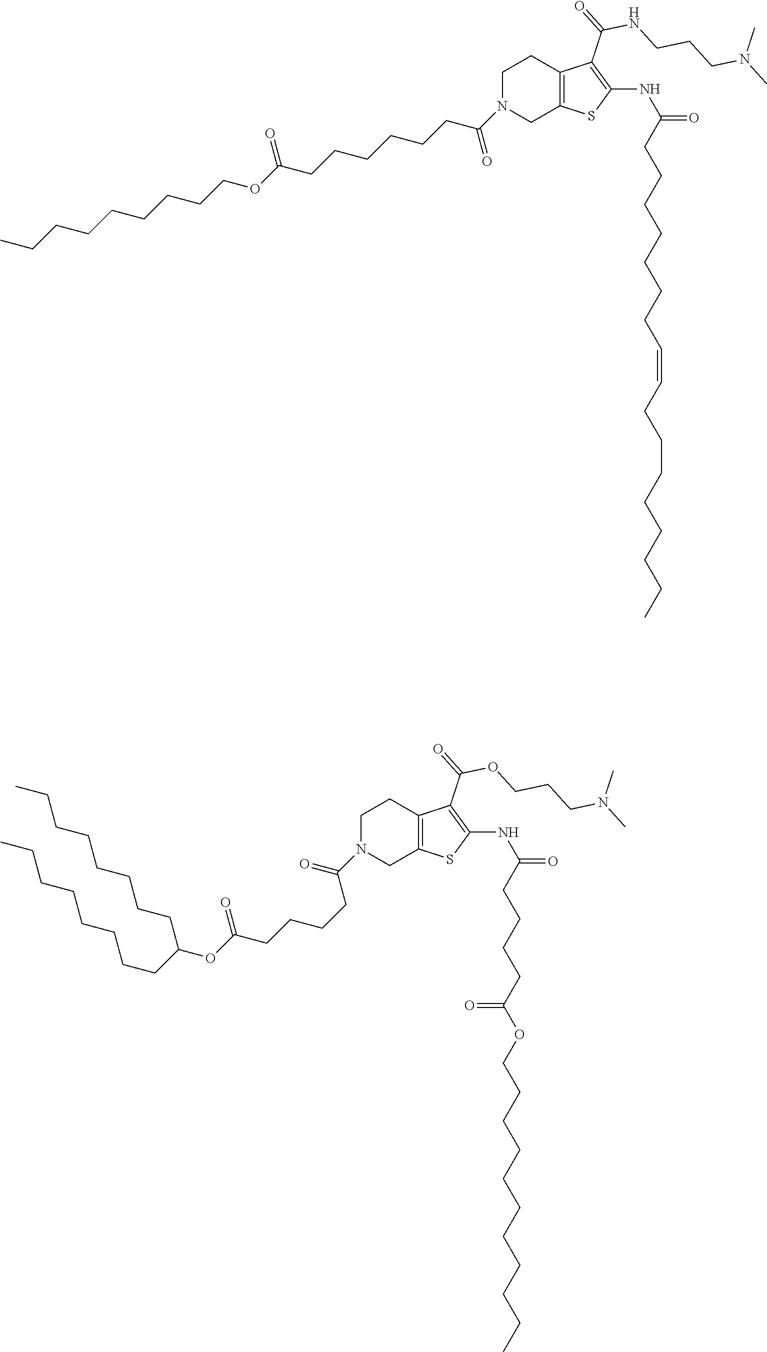

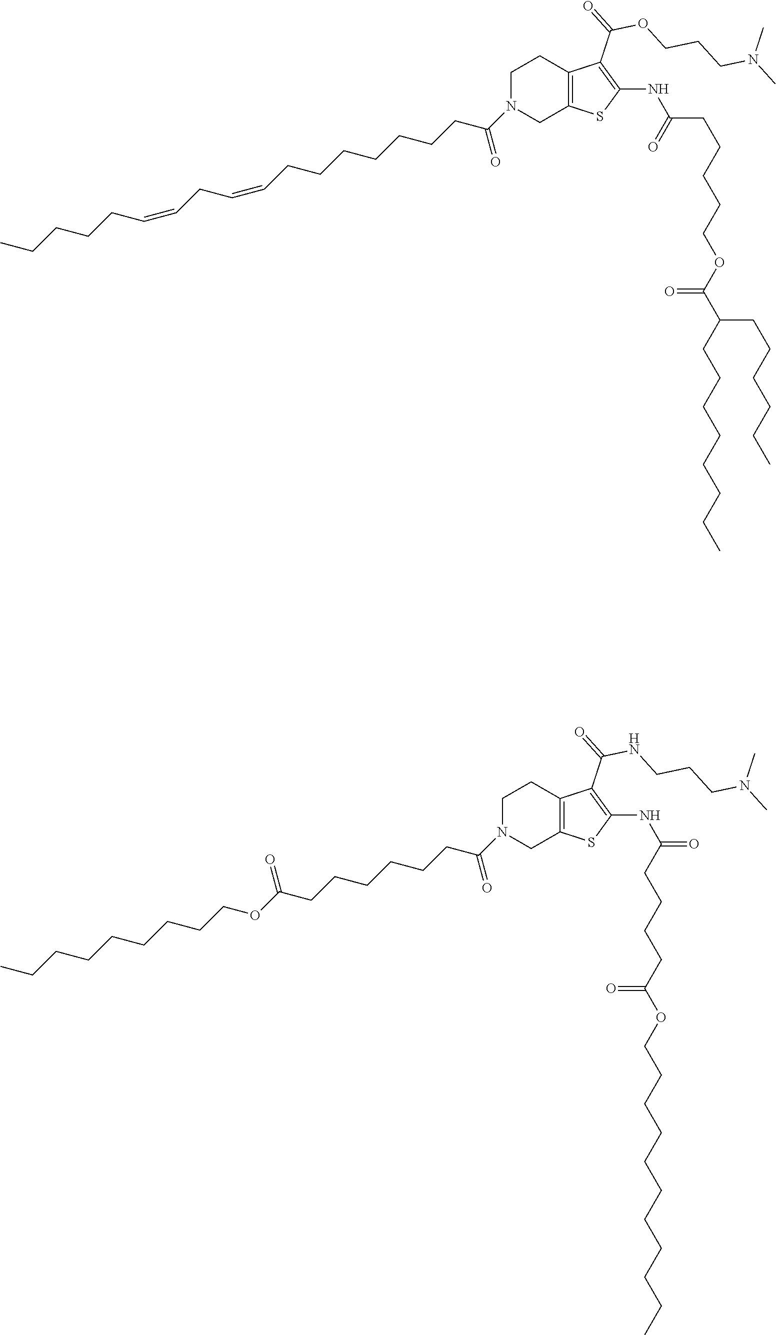

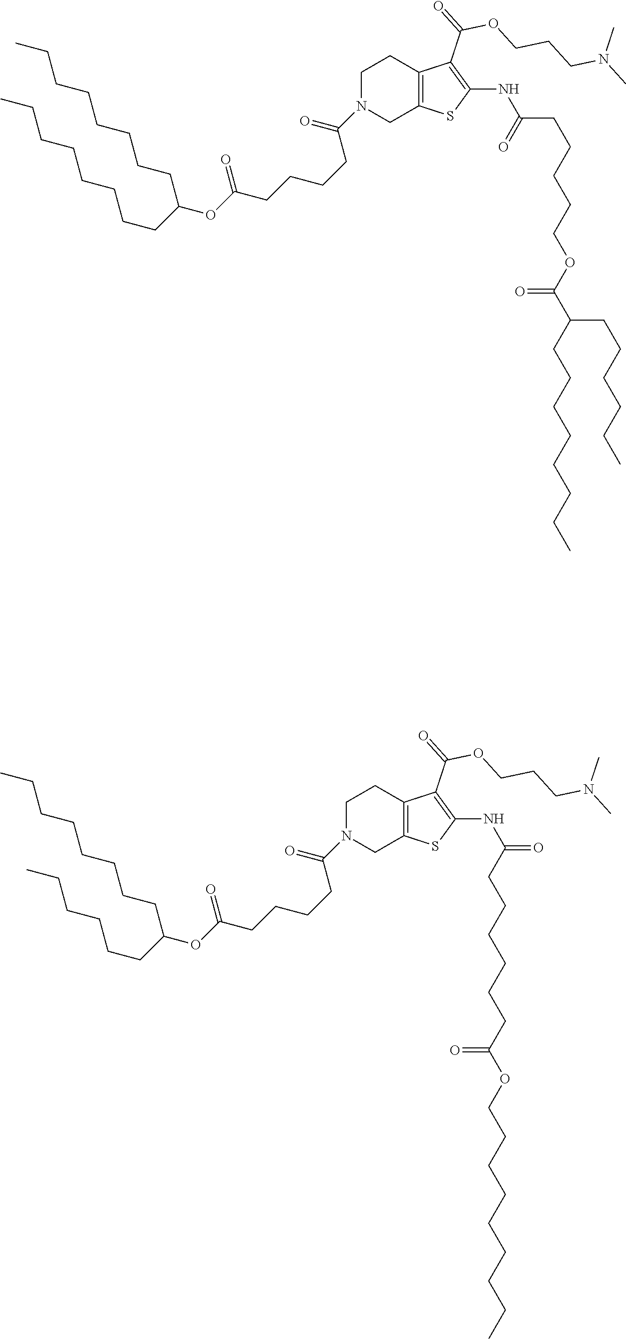

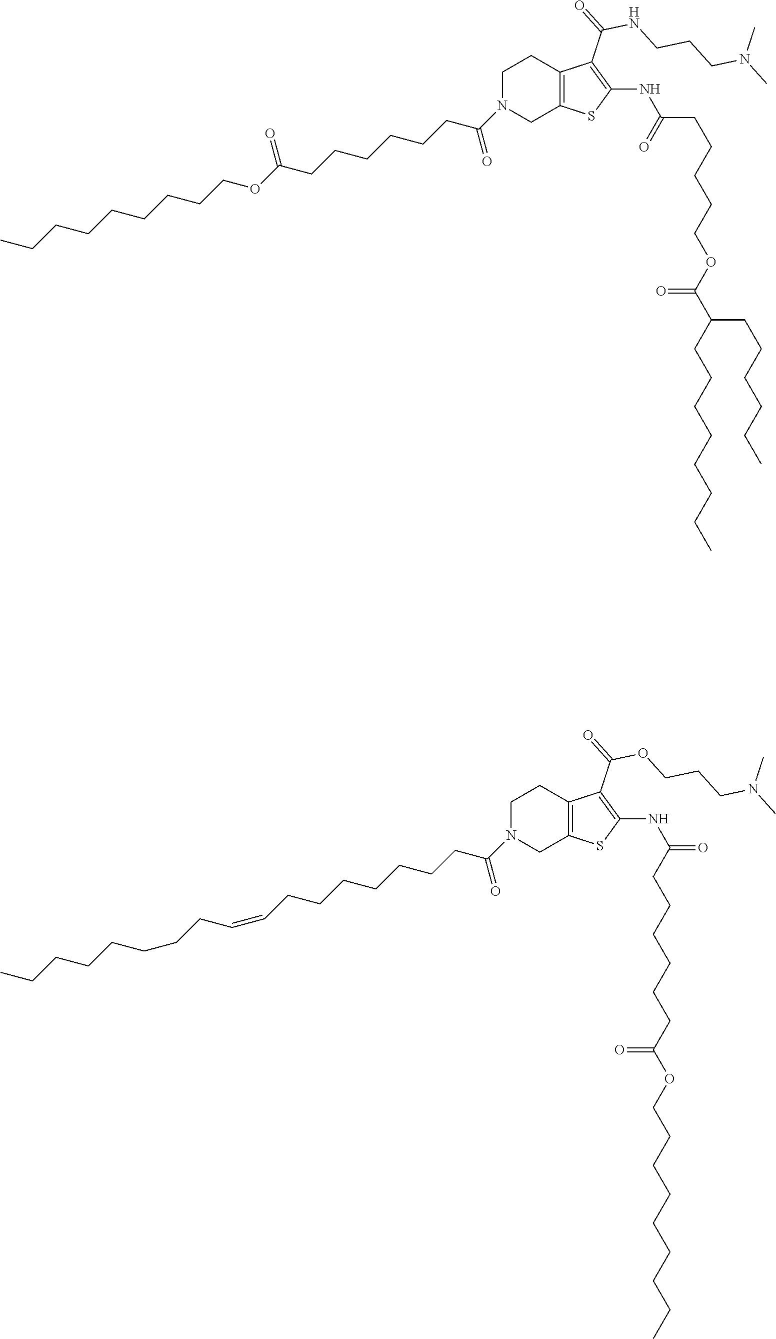

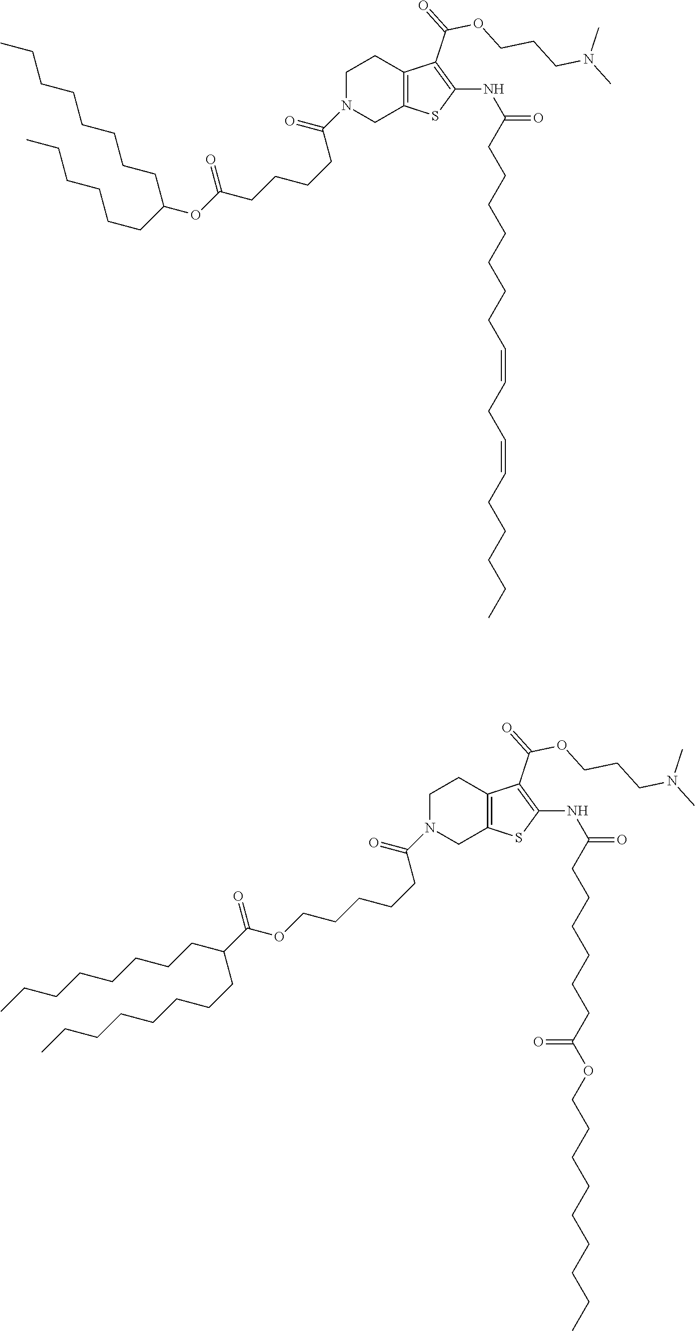

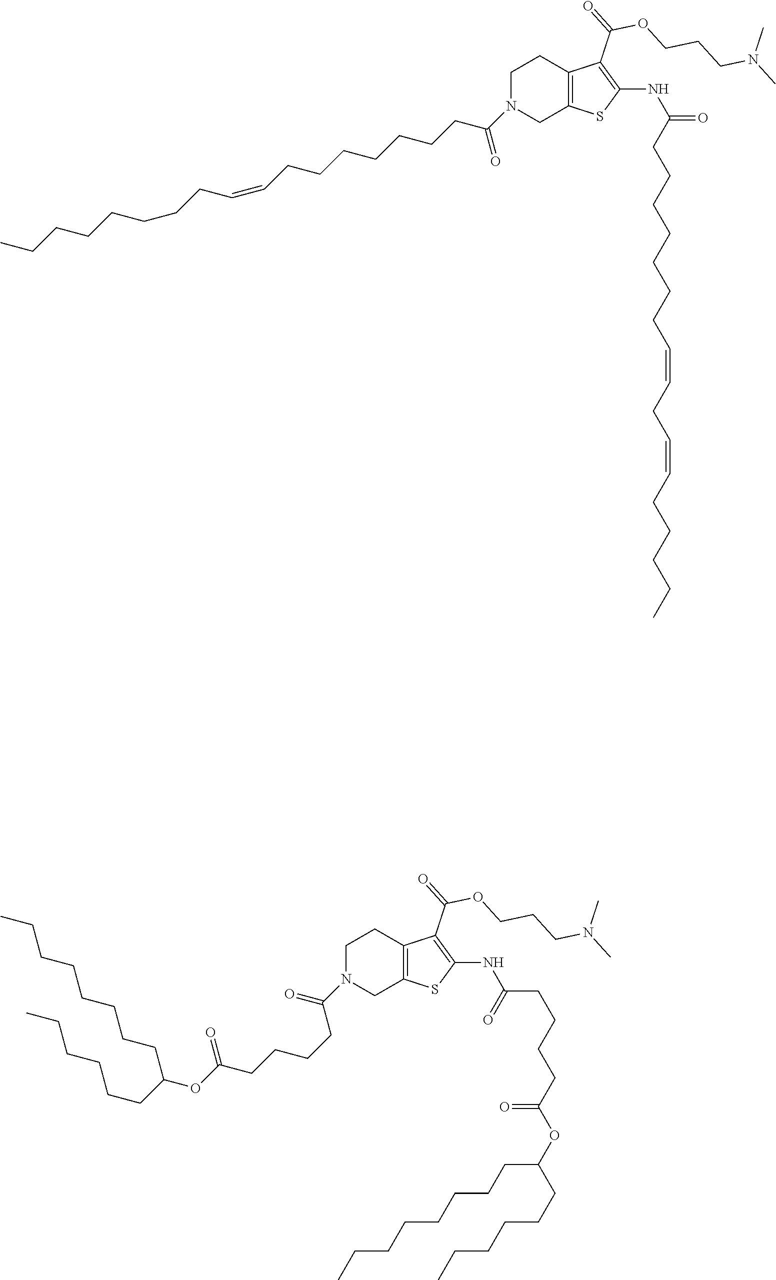

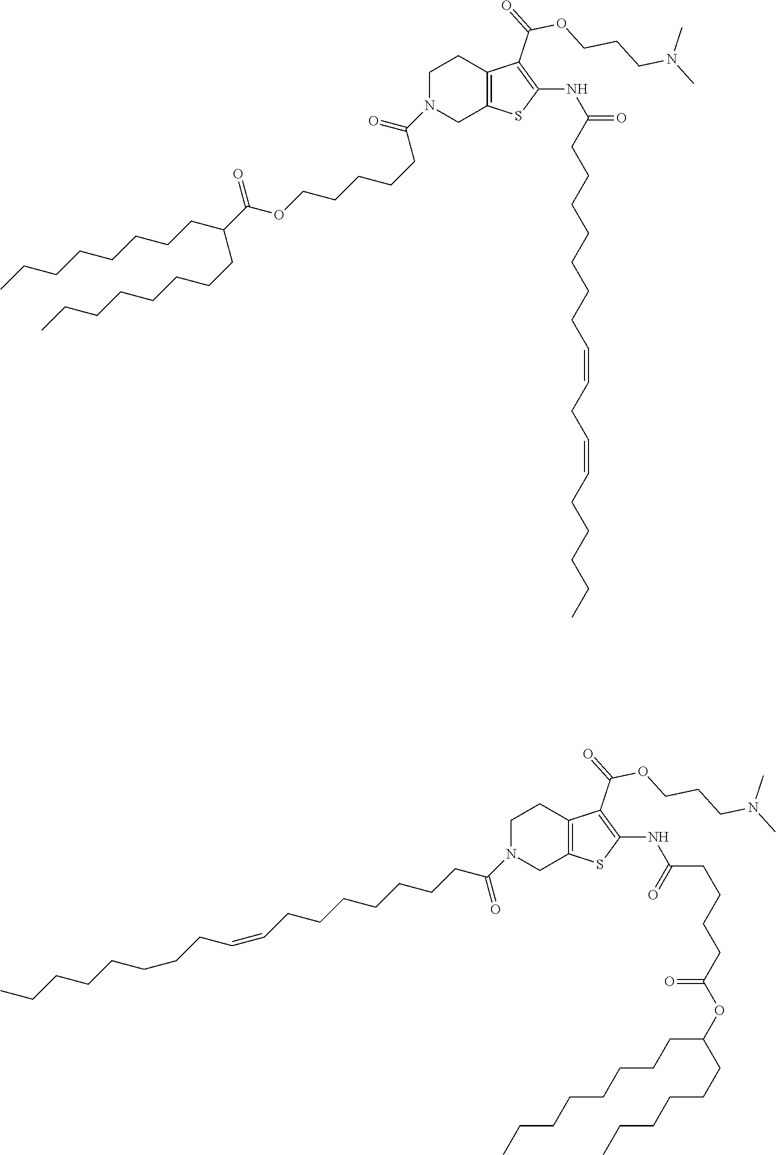

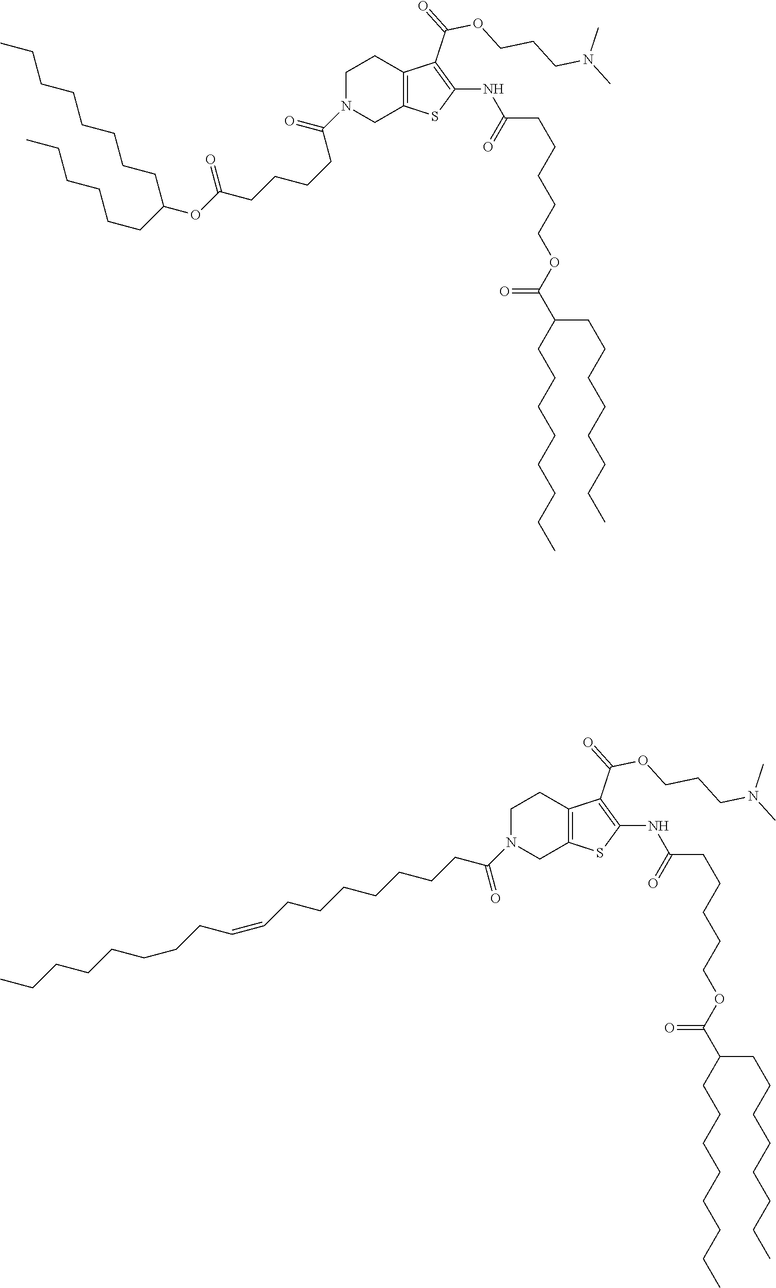

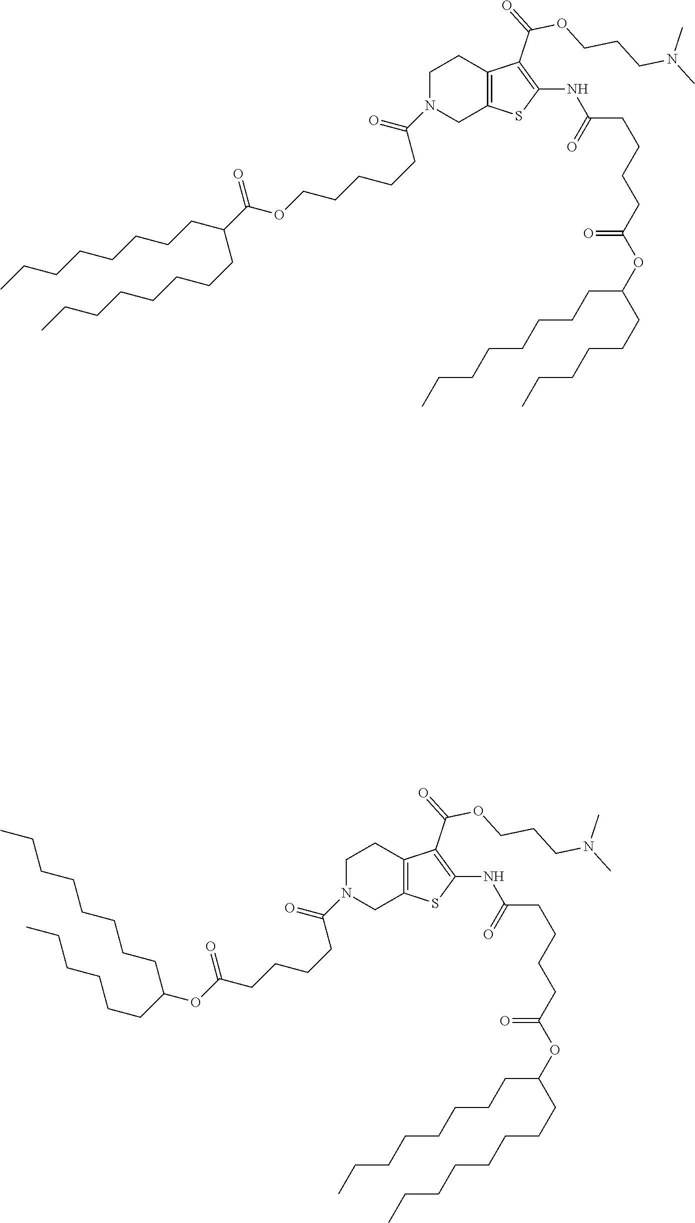

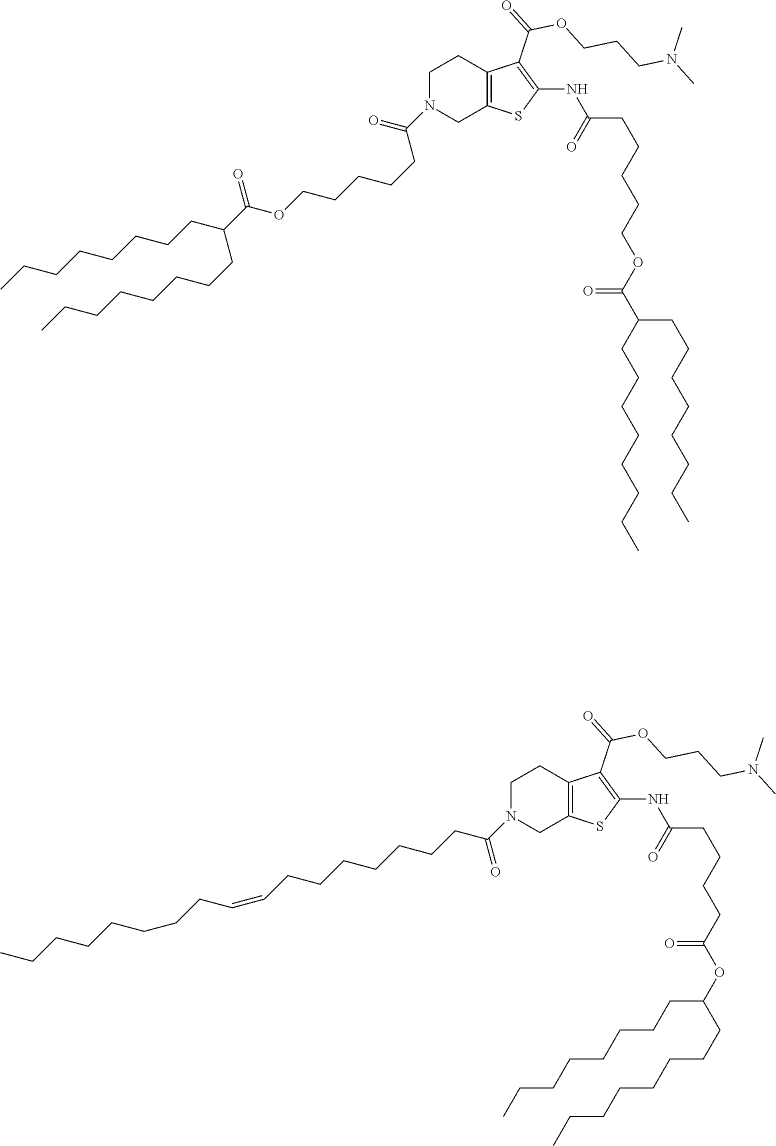

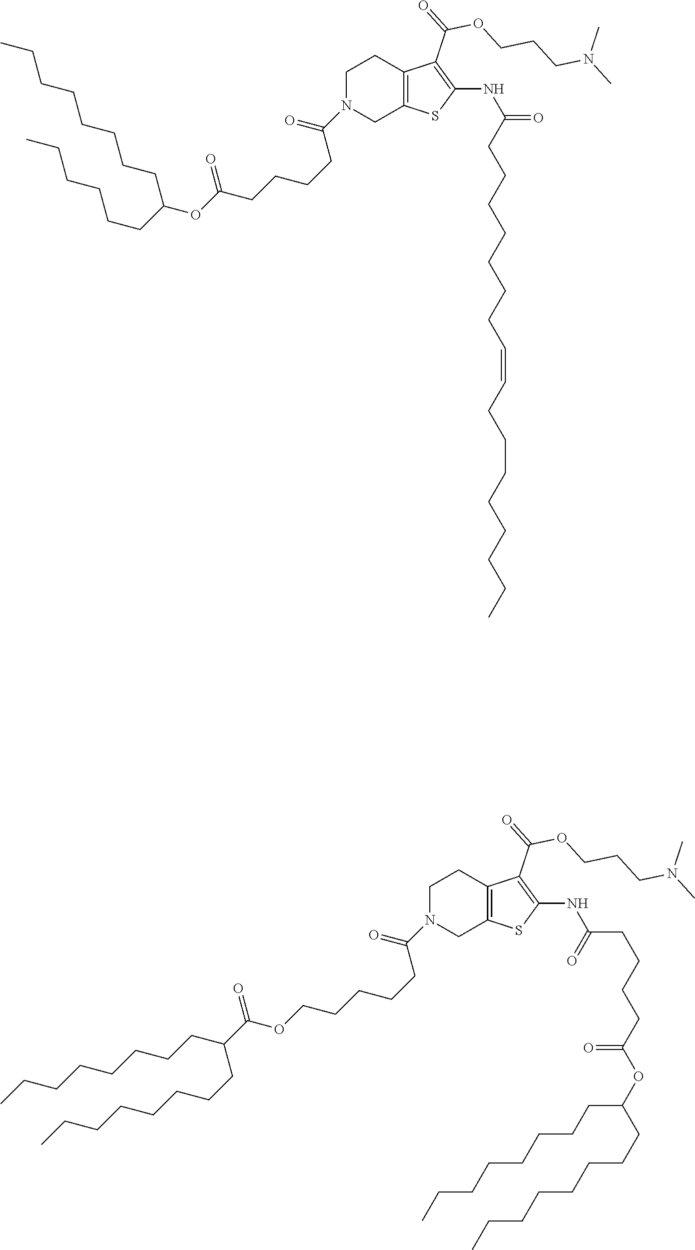

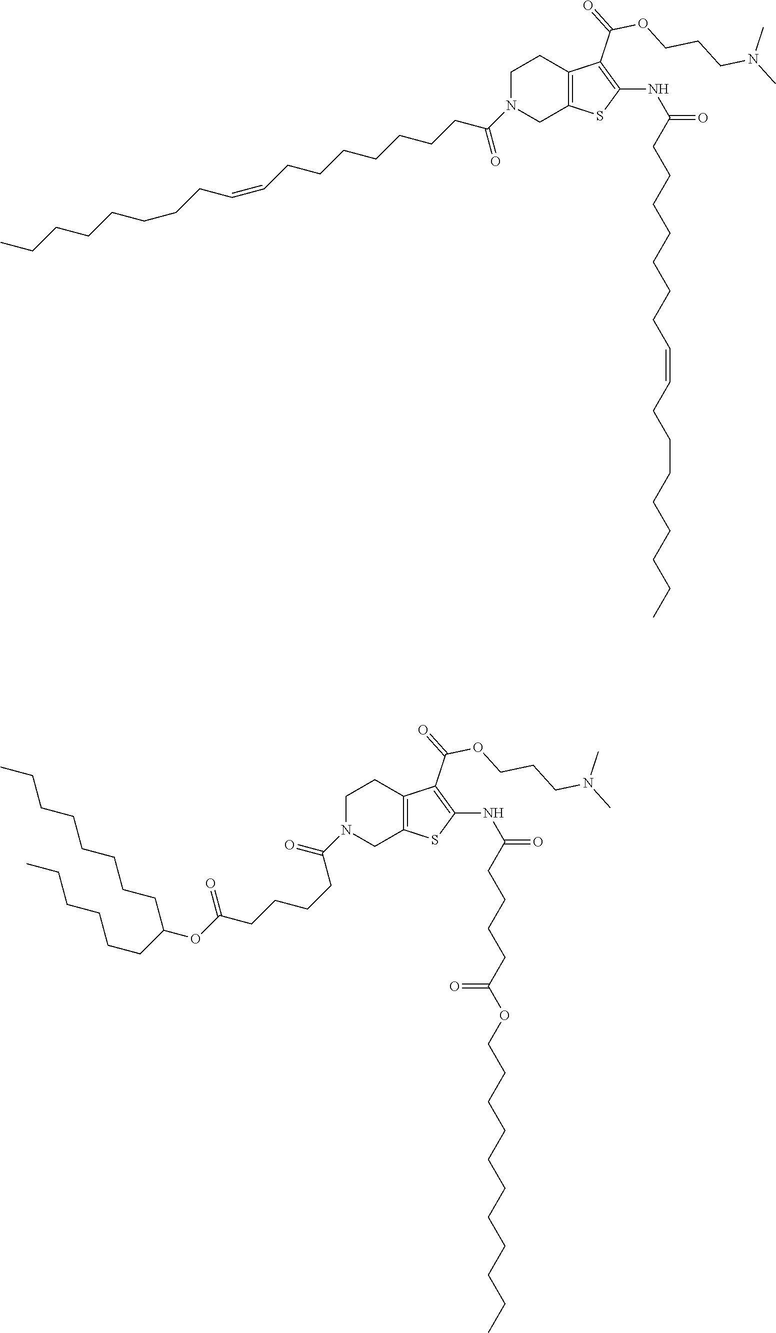

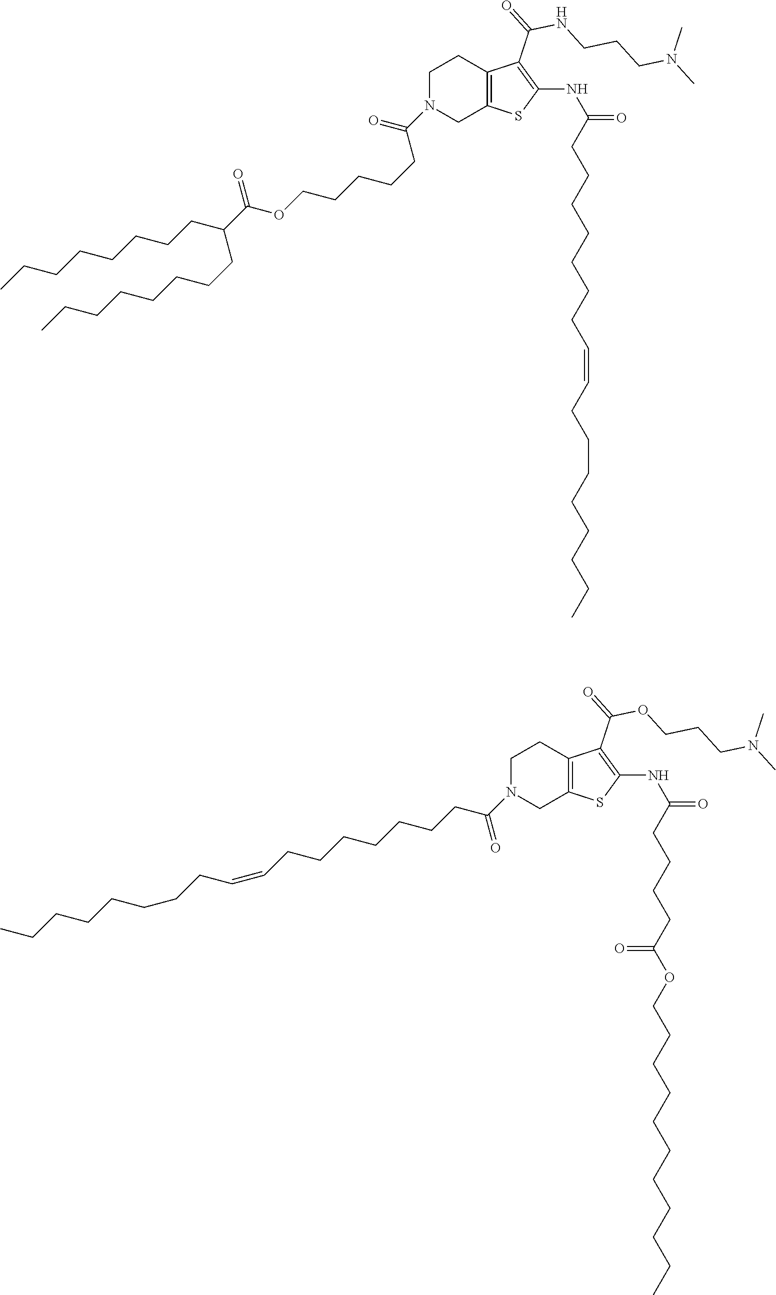

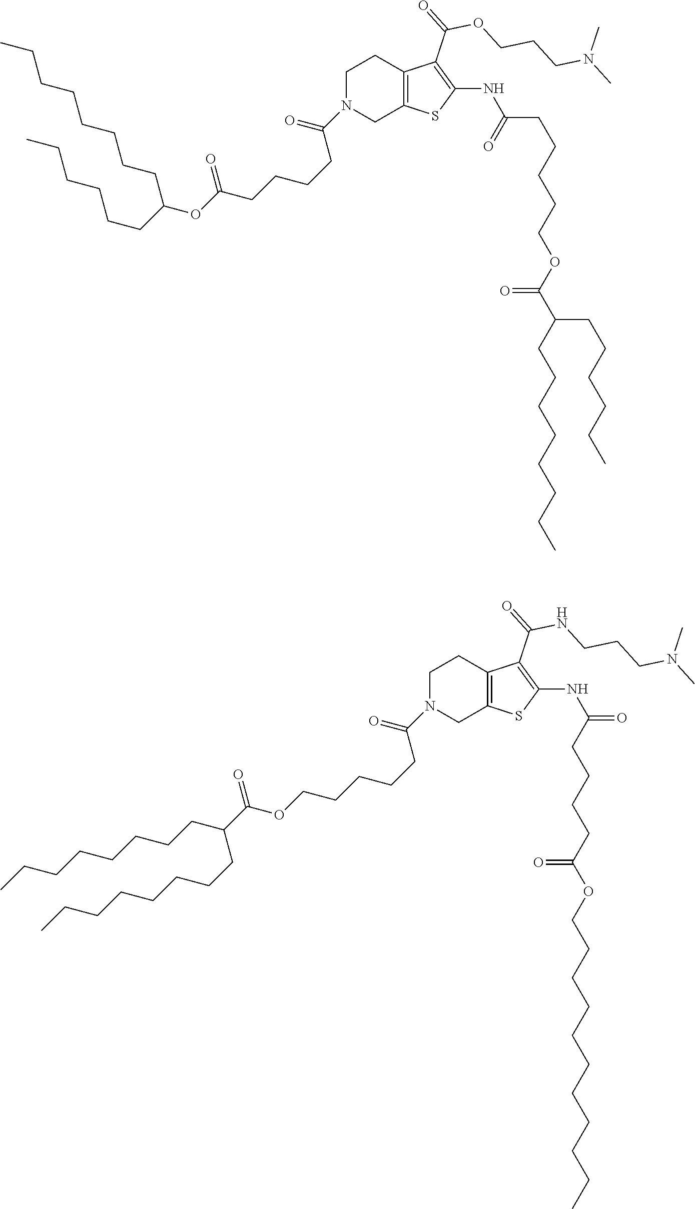

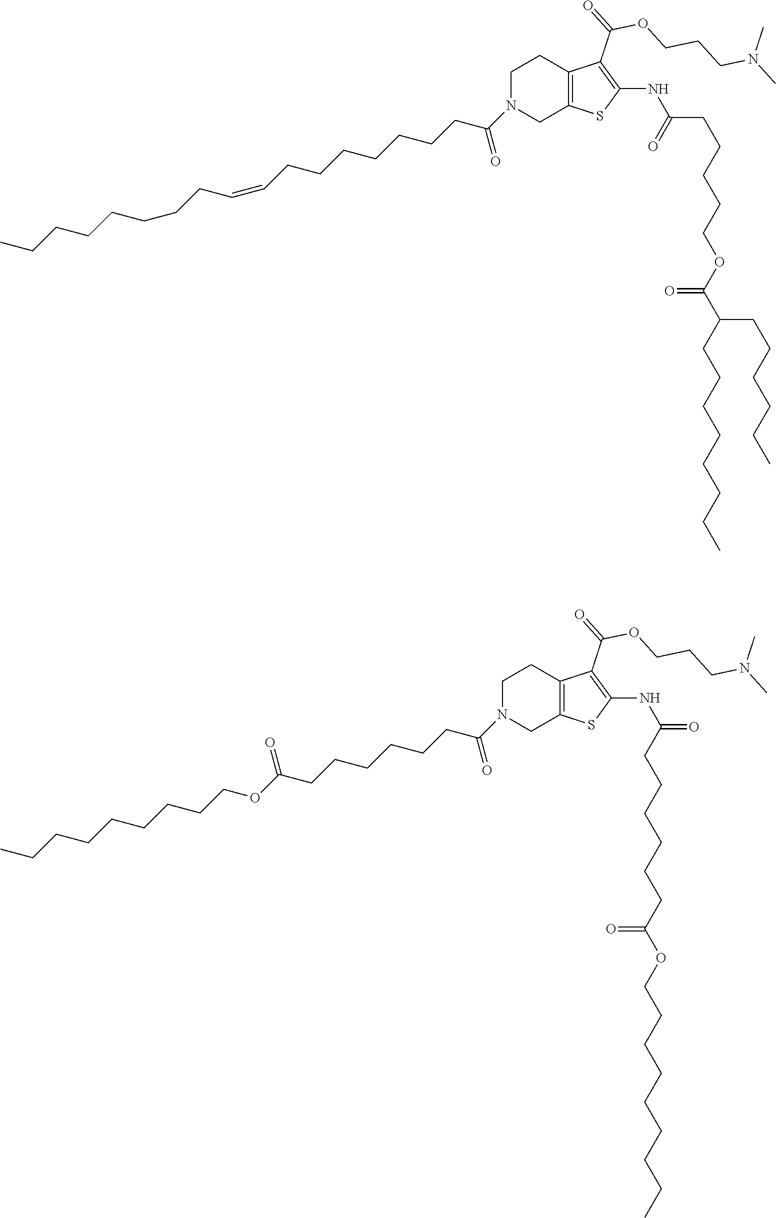

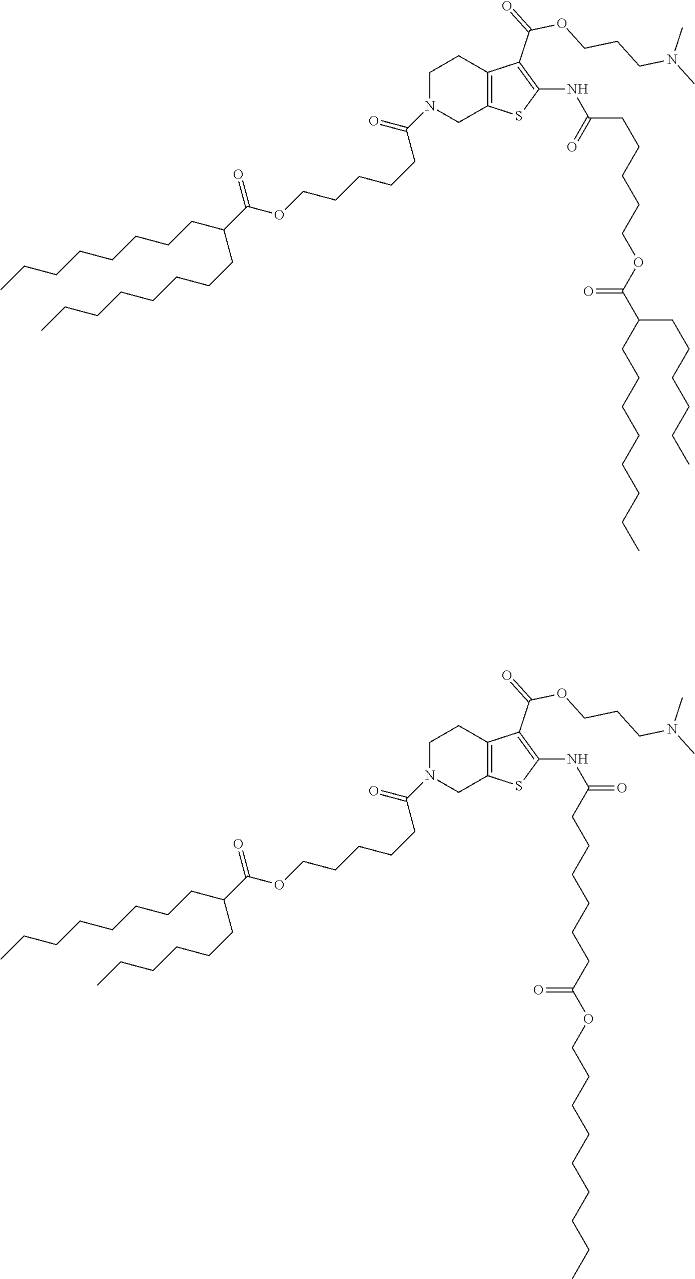

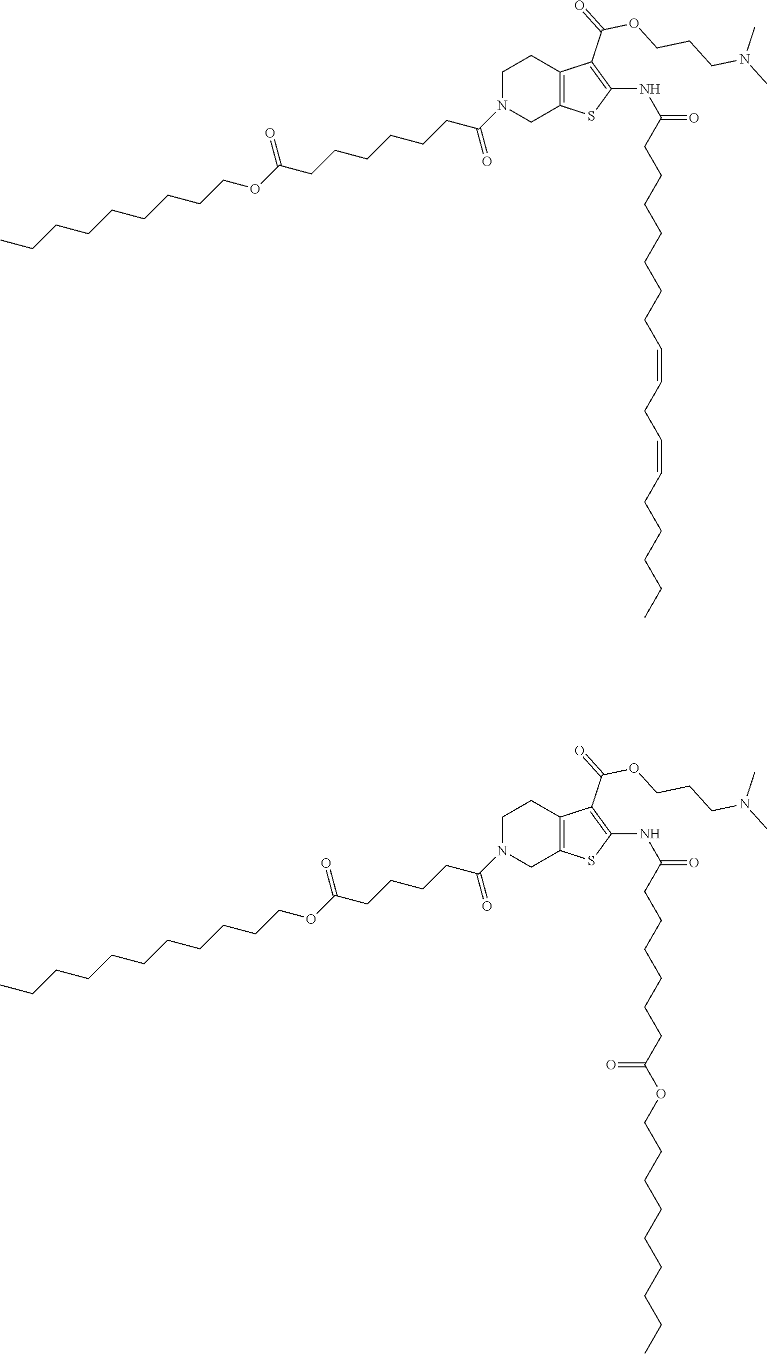

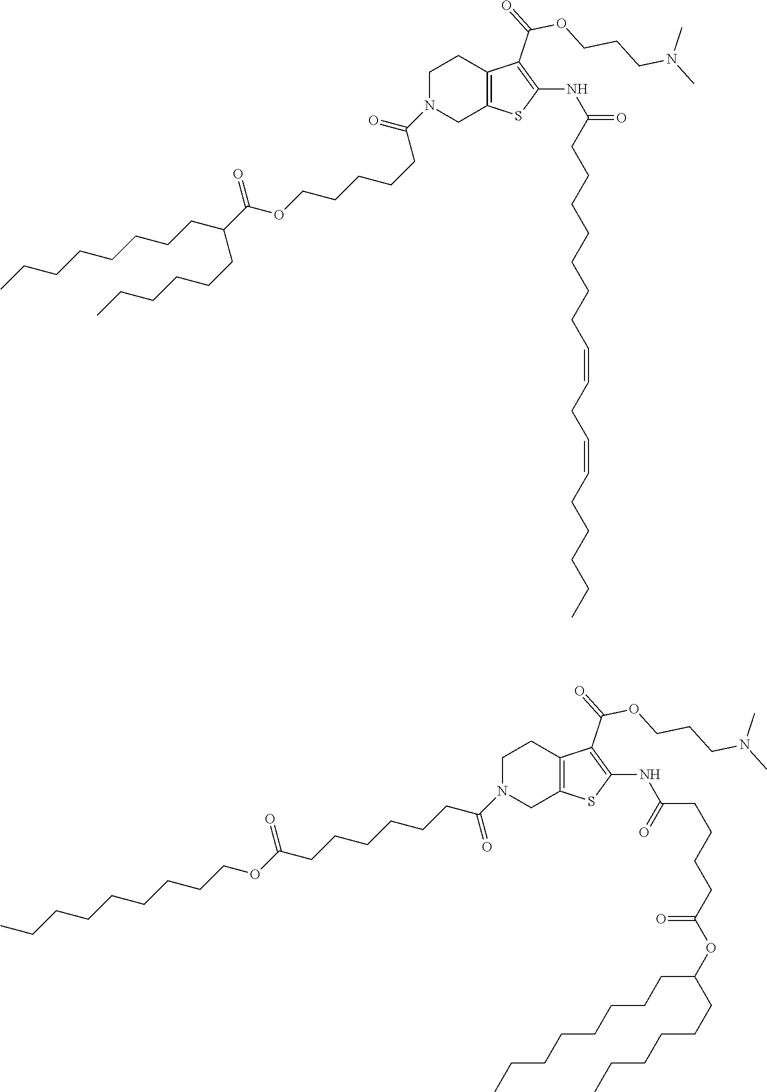

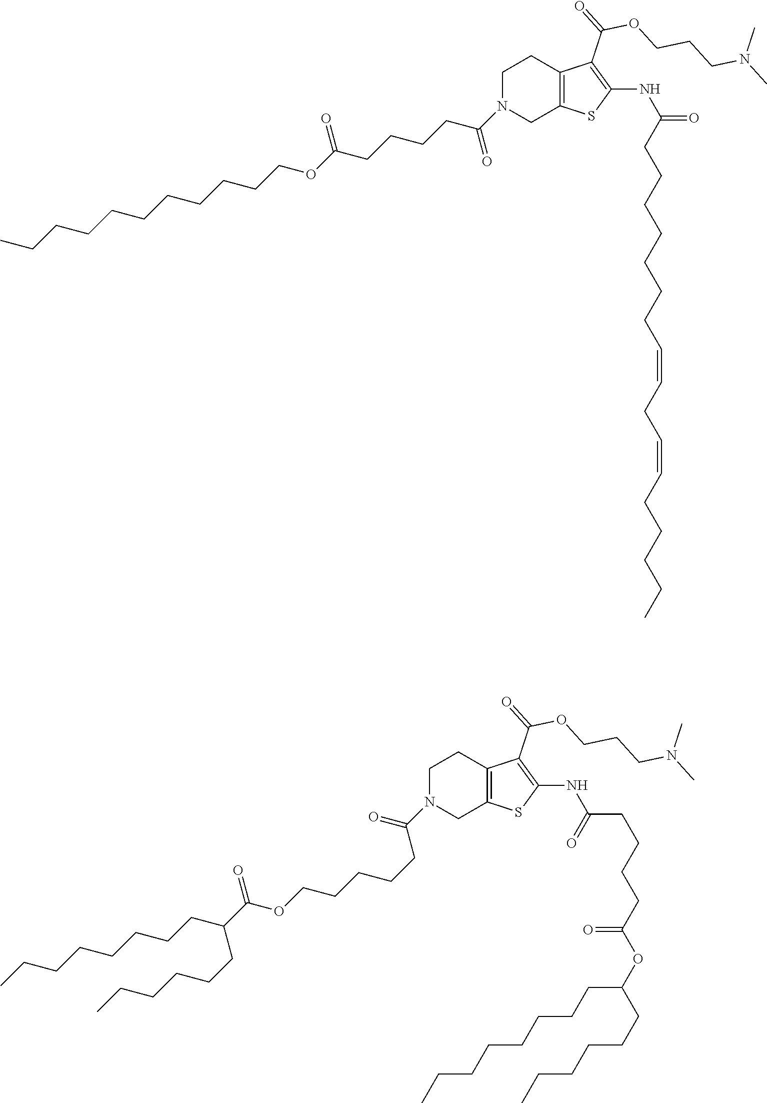

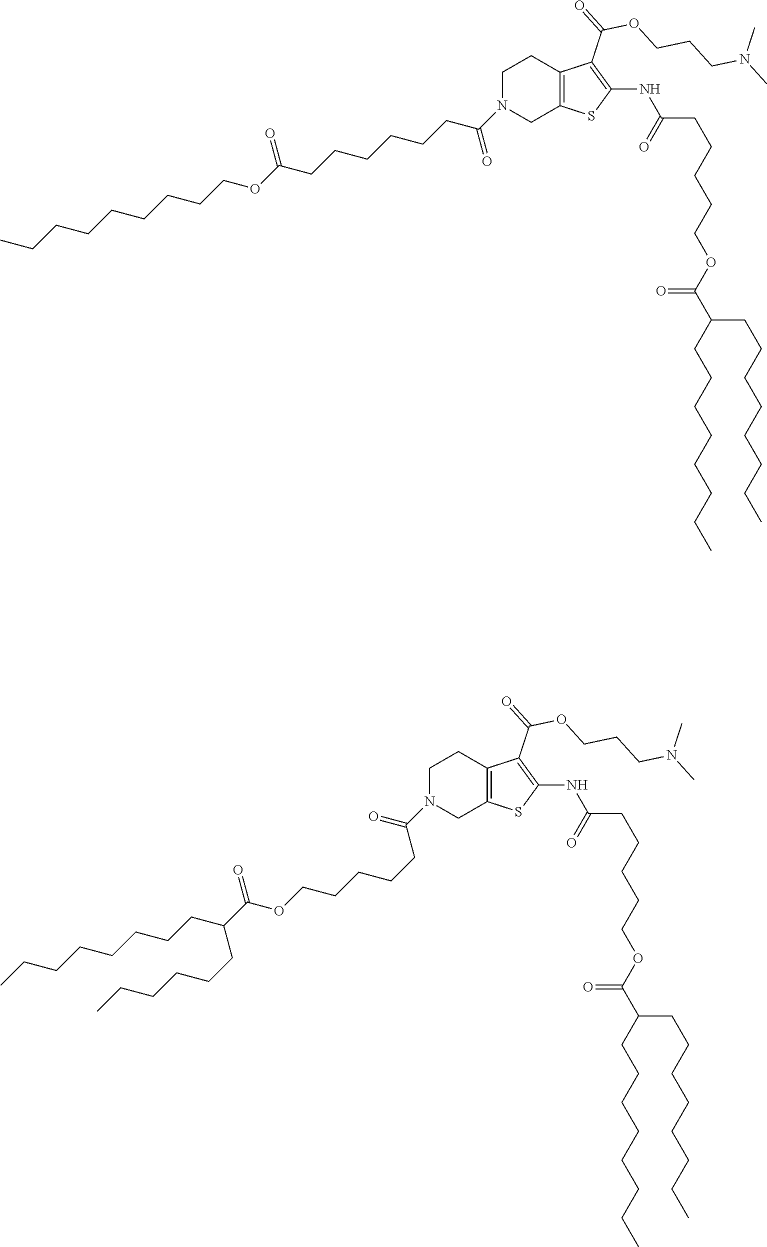

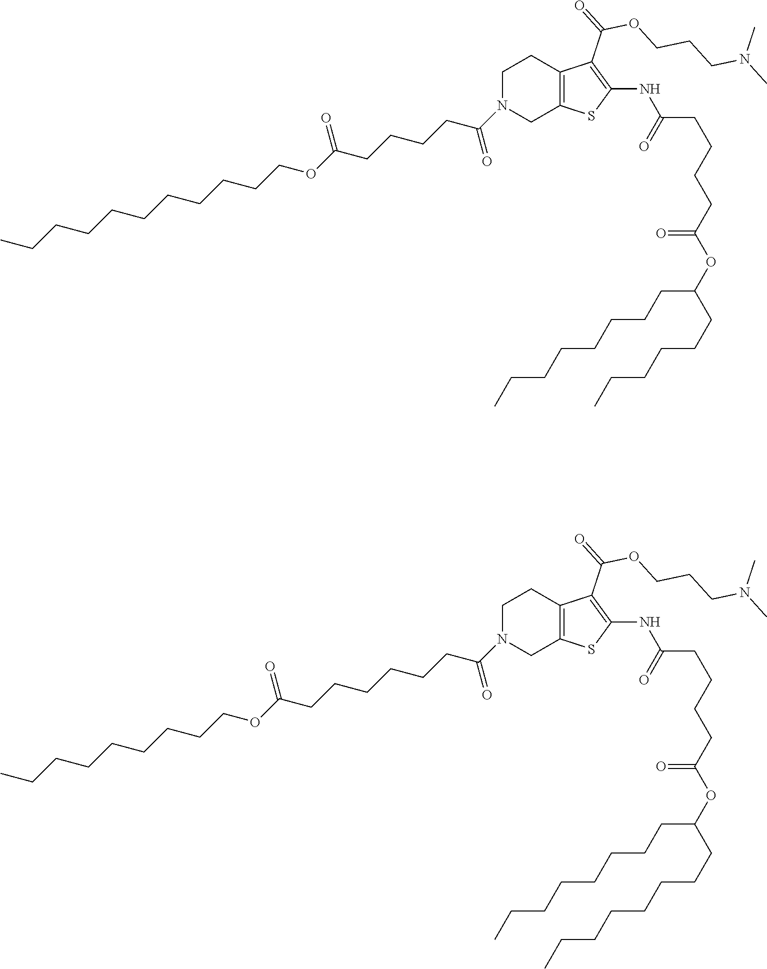

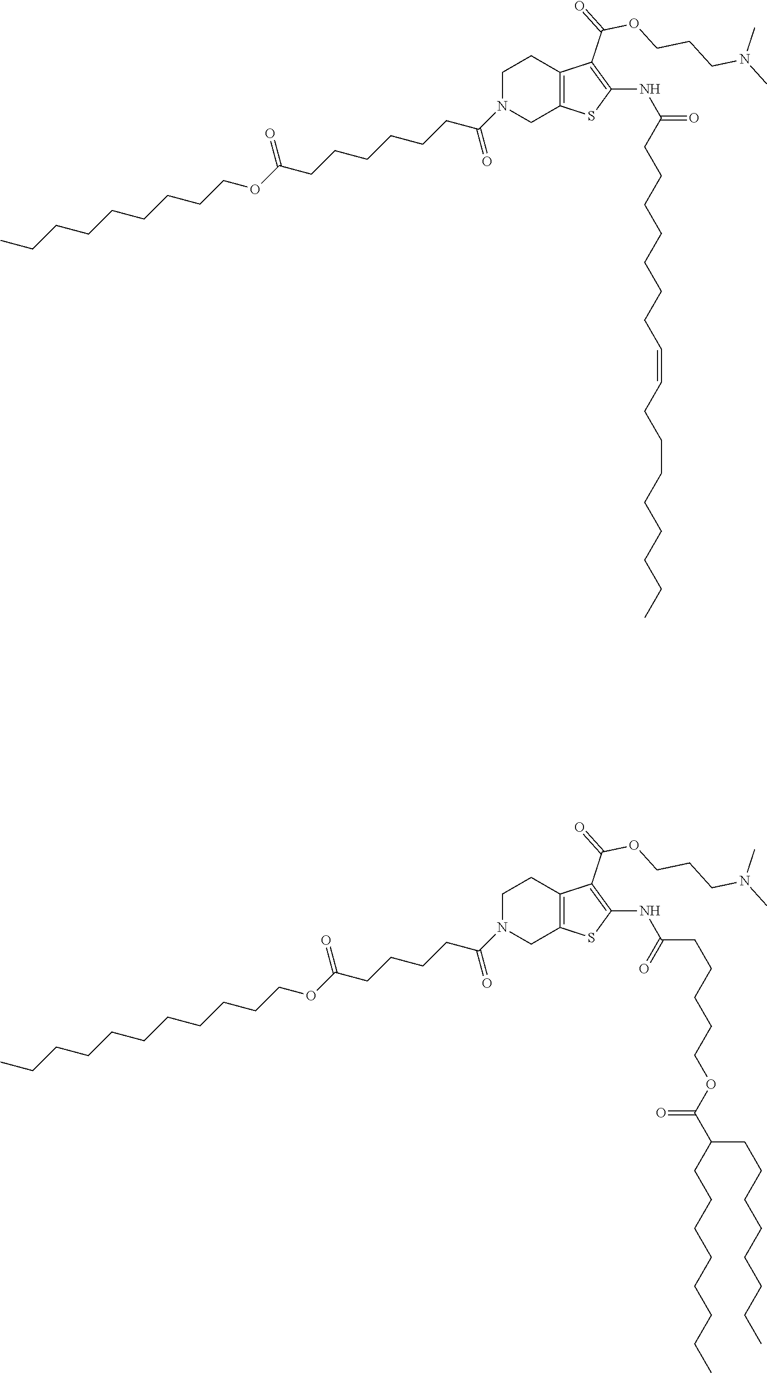

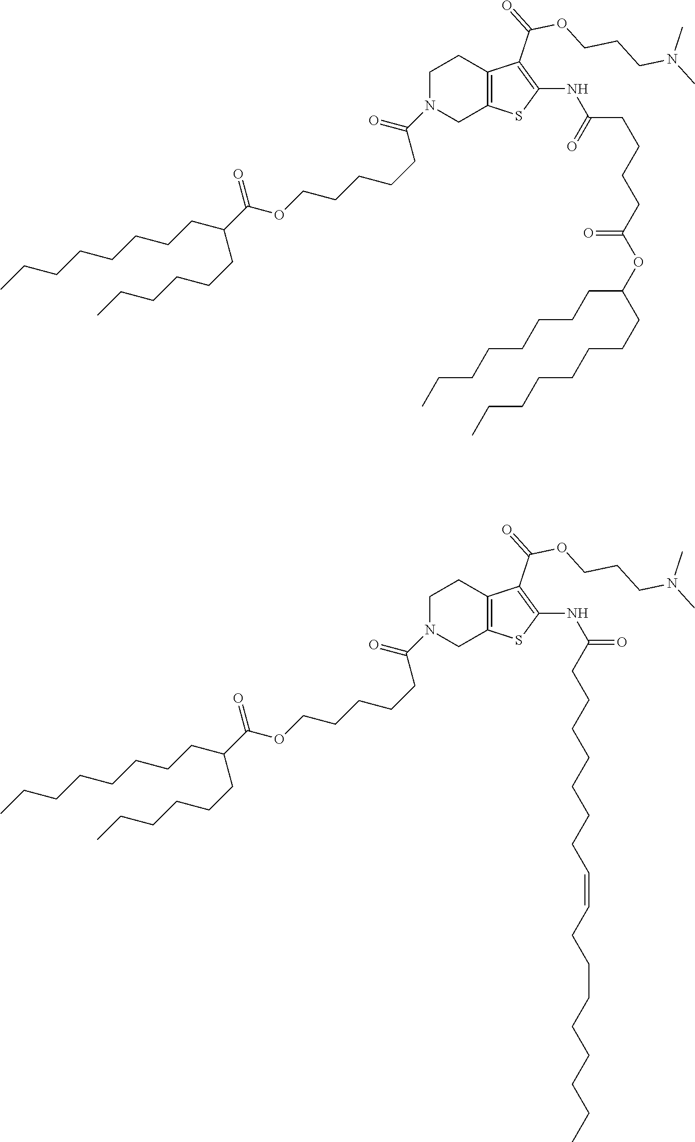

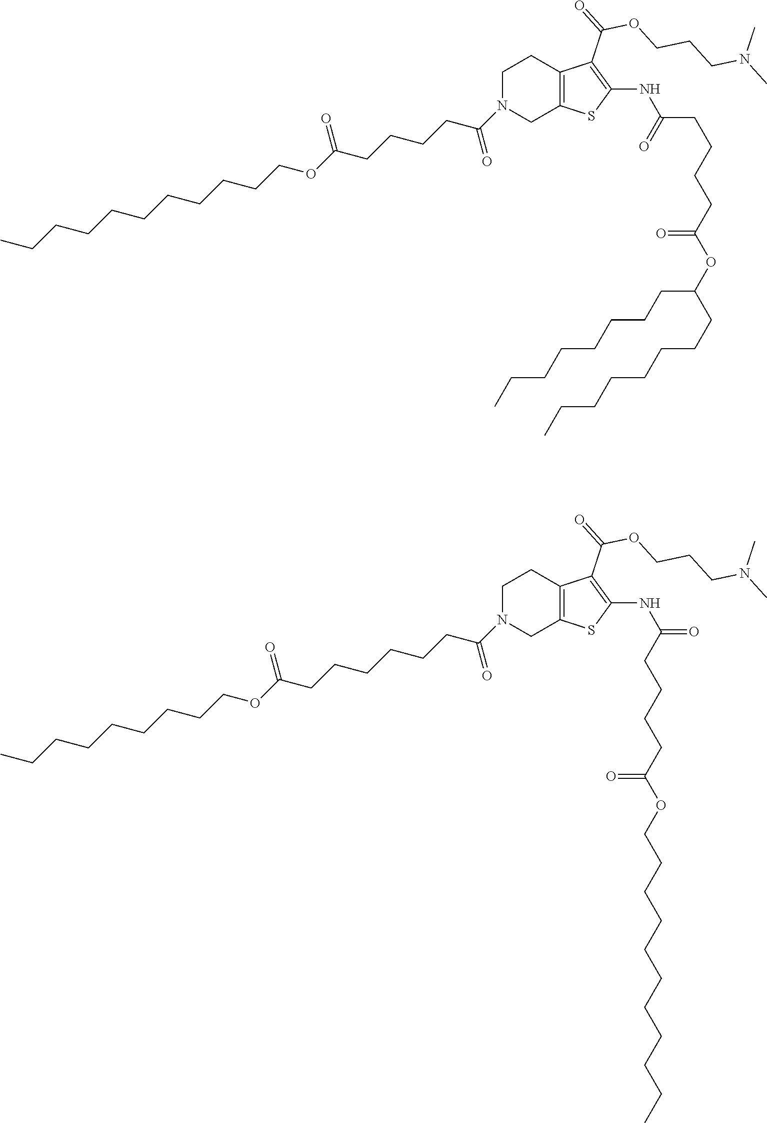

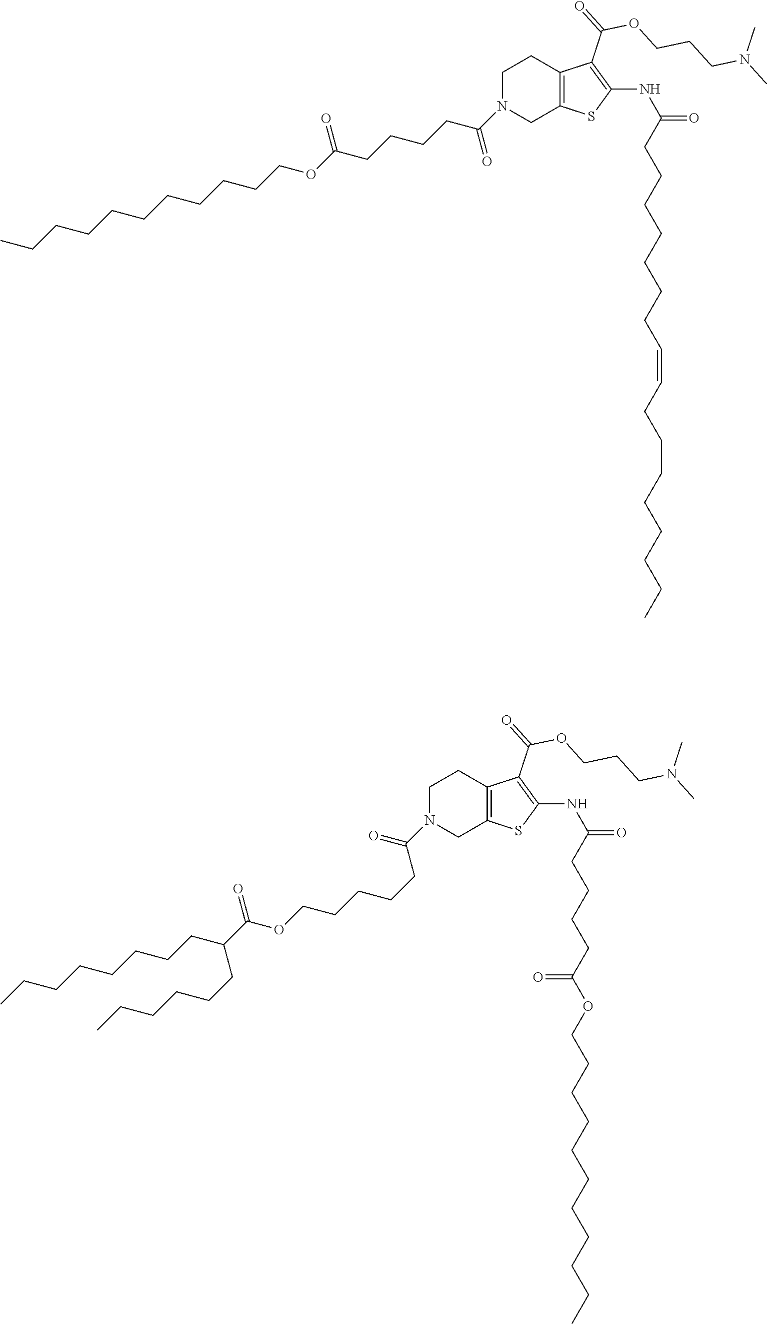

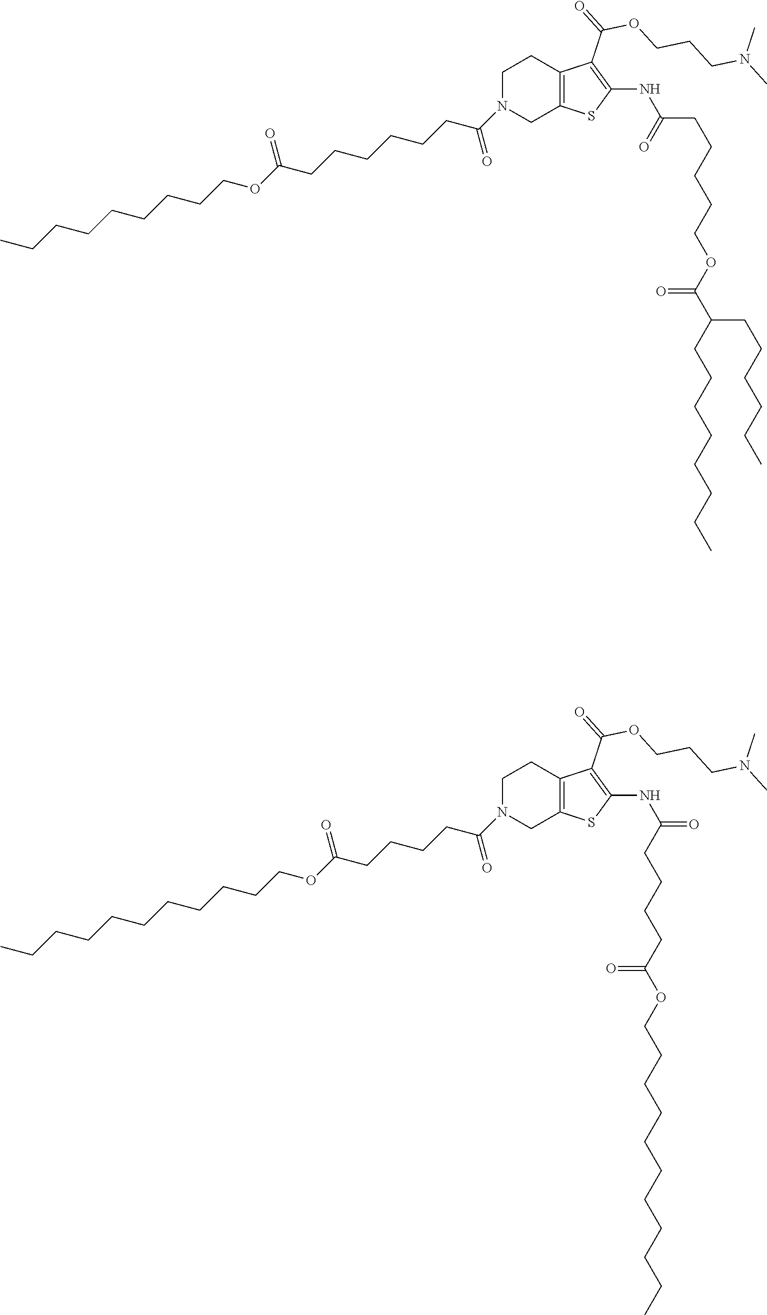

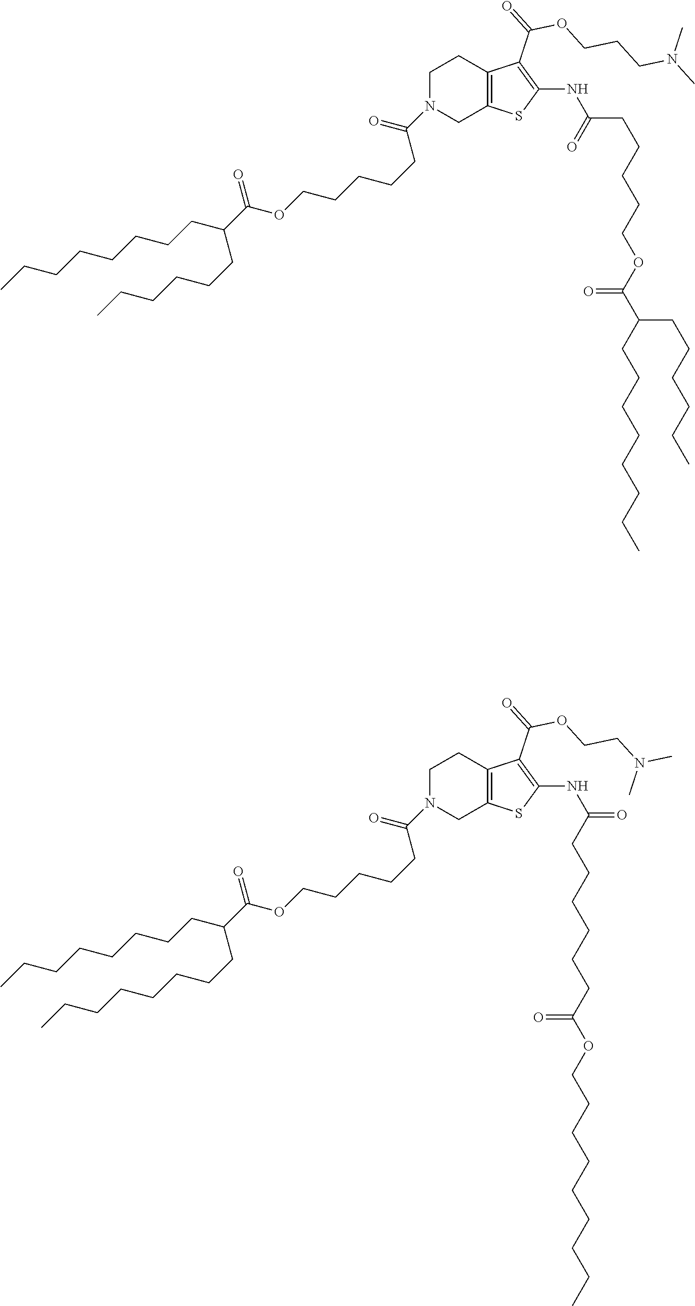

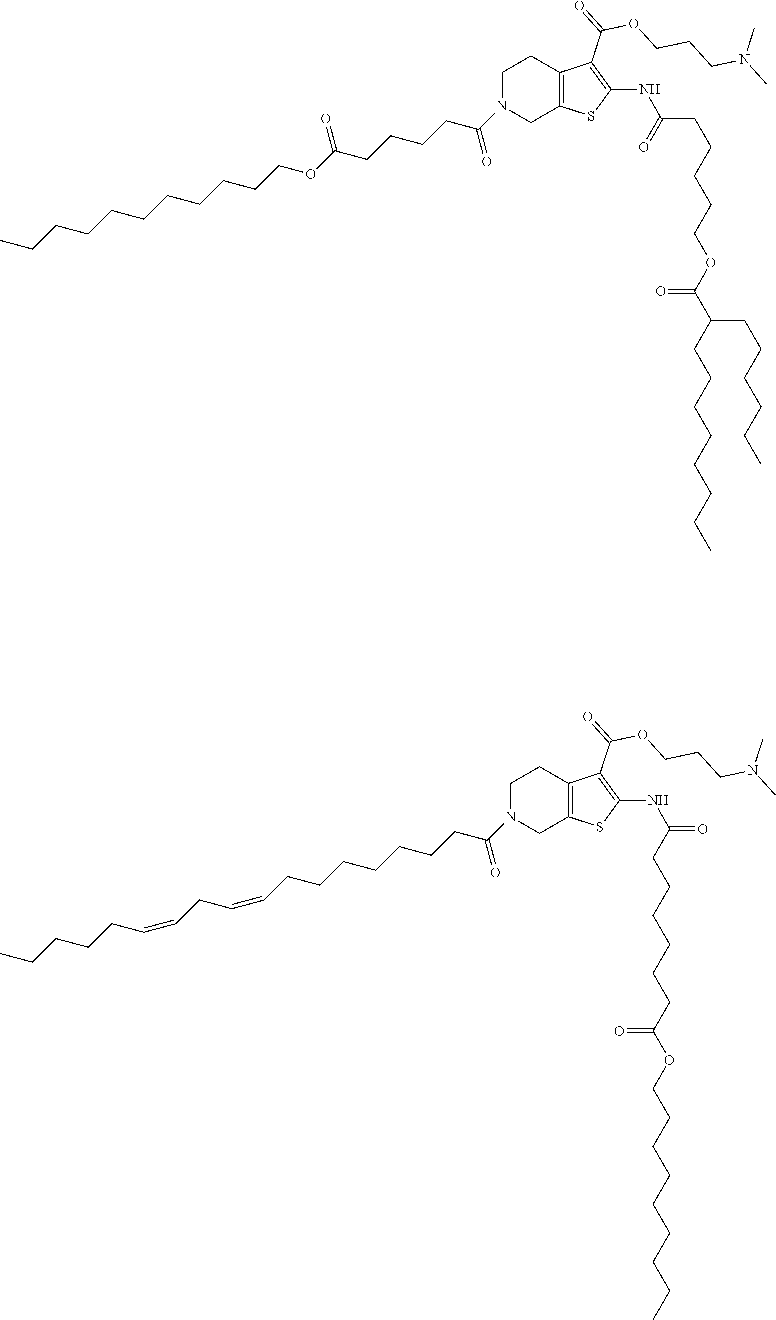

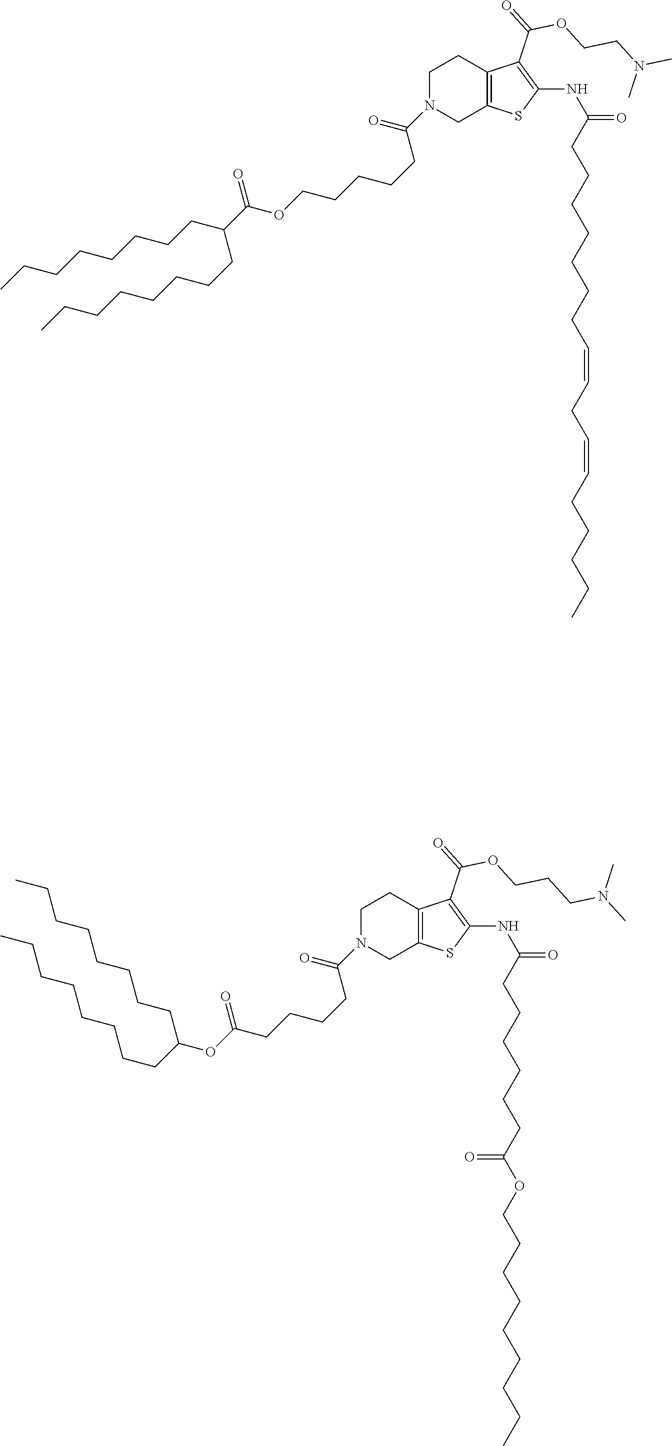

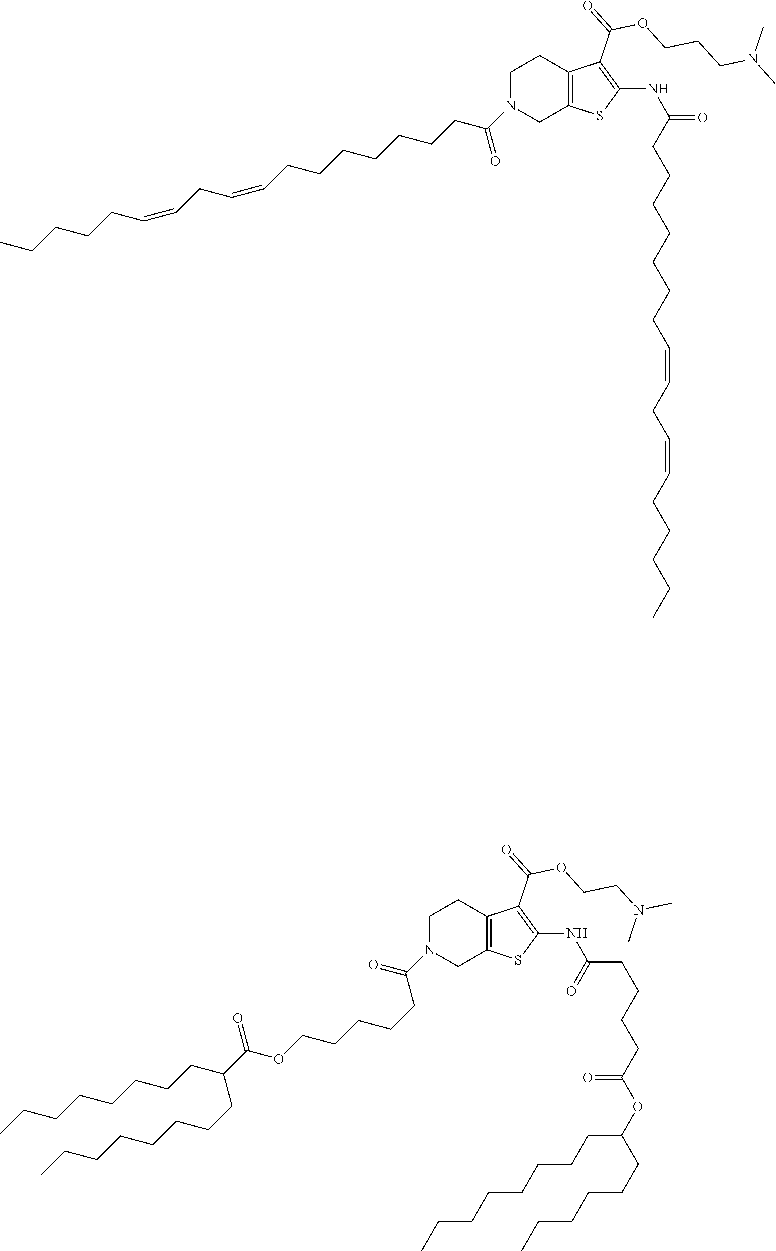

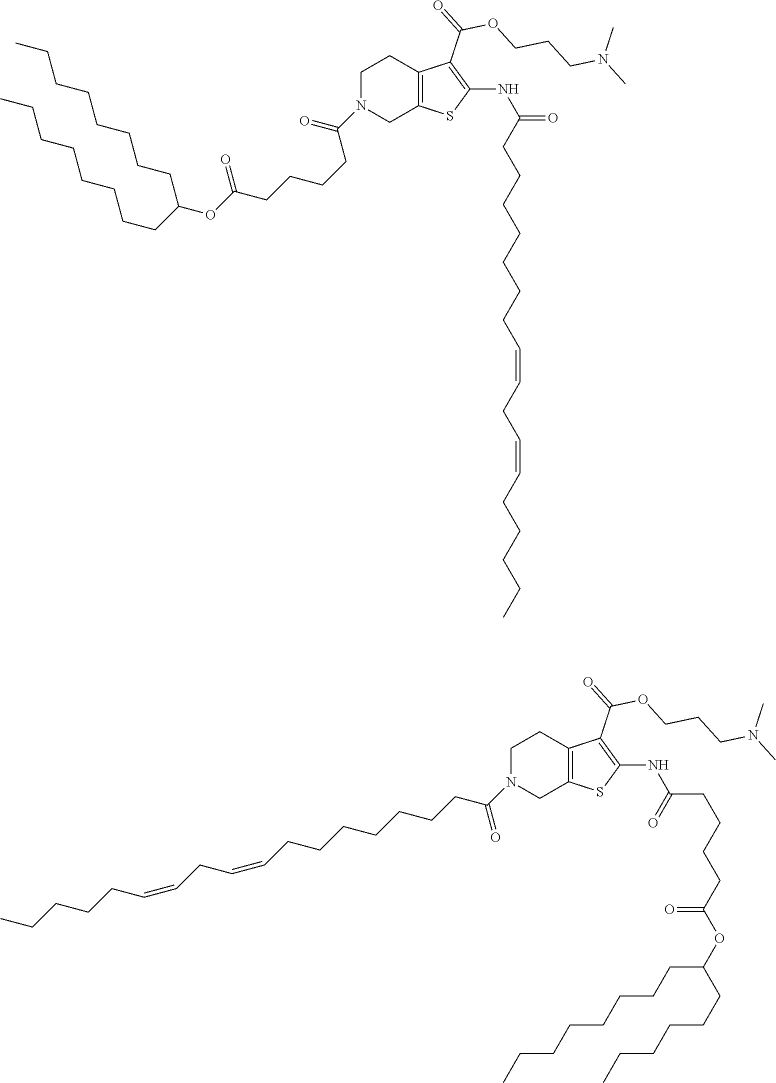

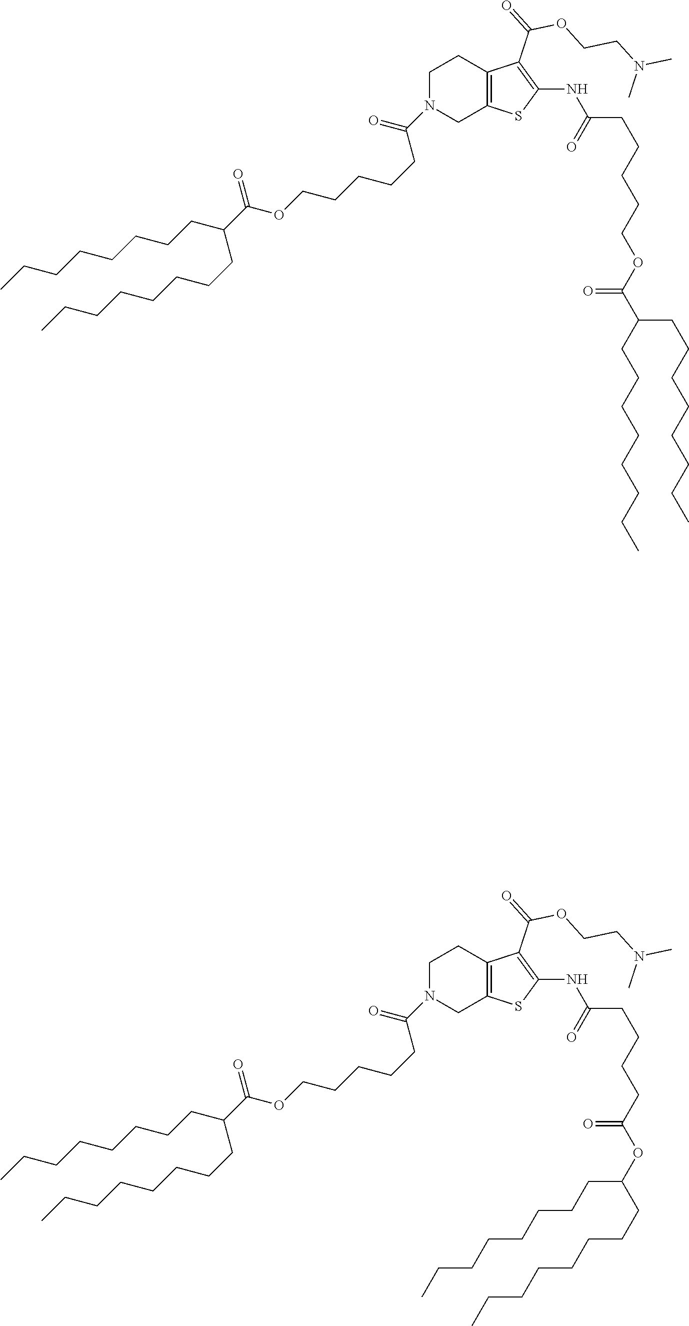

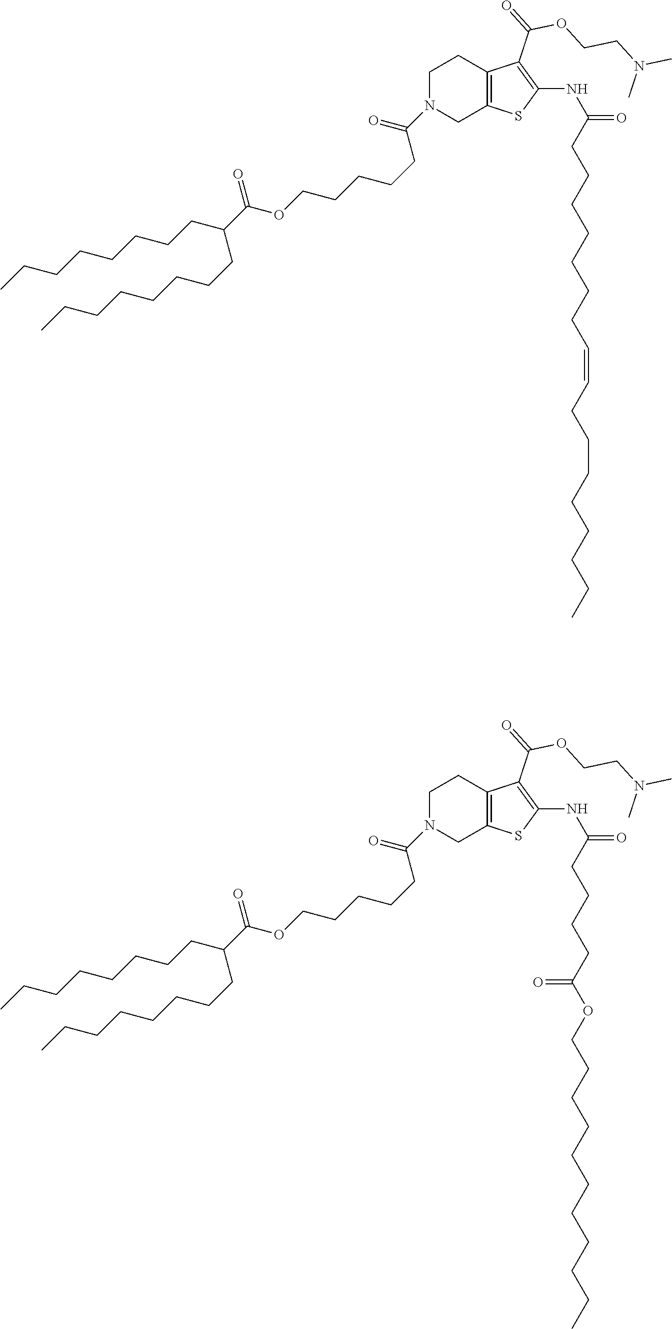

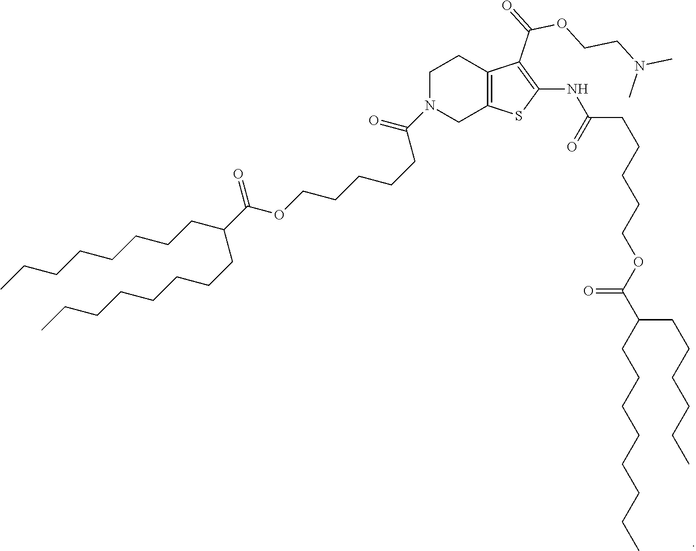 [SMILES: CCCCCCCCC(CCCCCC)C(=O)OCCCCCC(=O)Nc1sc2c(c1C(=O)OCCN(C)C)CCN(C(=O)CCCCCOC(=O)C(CCCCCCCC)CCCCCCCC)C2]

78. The compound of claim 1, wherein the compound is of the formula:

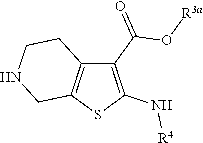

| | R^{3a} | | | |
|---|---|---|---|---|
| R⁴ | 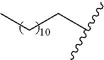 | 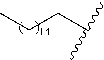 | 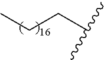 | 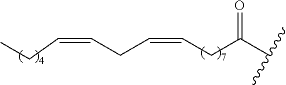 |
| 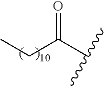 | 1-1 | 1-2 | 1-3 | 1-4 |
| 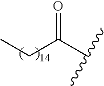 | 2-1 | 2-2 | 2-3 | 2-4 |
| 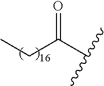 | 3-1 | 3-2 | 3-3 | |
| 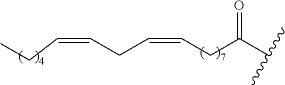 | 4-1 | 4-2 | 4-3 | |
| 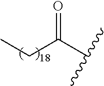 | 5-1 | 5-2 | 5-3 | 5-4 |
| 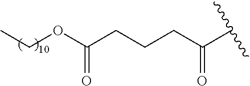 | 6-1 | 6-2 | 6-3 | 6-4 |
| 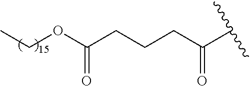 | 7-1 | 7-2 | 7-3 | 7-4 |
| 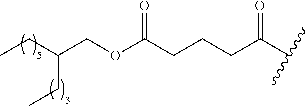 | 8-1 | 8-2 | 8-3 | 8-4 |
| 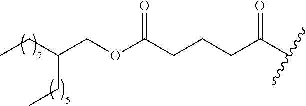 | 9-1 | 9-2 | 9-3 | 9-4 |
| 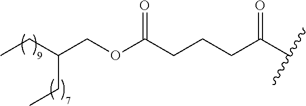 | 10-1 | 10-2 | 10-3 | 10-4 |
| 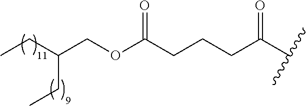 | 11-1 | 11-2 | 11-3 | 11-4 |
| | R^{3a} | | |
|---|---|---|---|
| R⁴ | 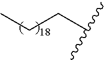 | 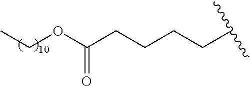 | 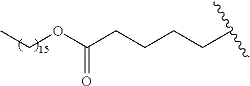 |
| 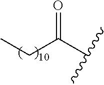 | 1-5 | 1-6 | 1-7 |
| 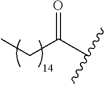 | 2-5 | 2-6 | 2-7 |
| 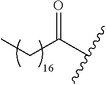 | 3-5 | 3-6 | 3-7 |
| 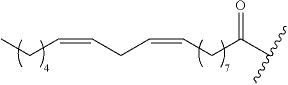 | 4-5 | 4-6 | 4-7 |
| 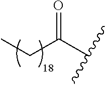 | 5-5 | 5-6 | 5-7 |
| 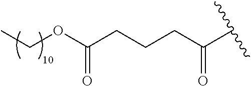 | 6-5 | 6-6 | 6-7 |
| 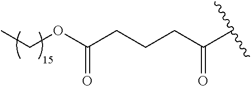 | 7-5 | 7-6 | 7-7 |
| 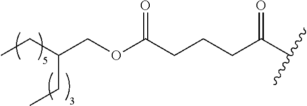 | 8-5 | 8-6 | 8-7 |
| 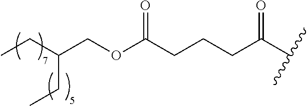 | 9-5 | 9-6 | 9-7 |
| 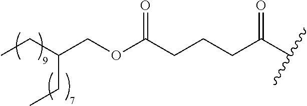 | 10-5 | 10-6 | 10-7 |
| 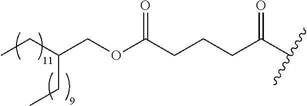 | 11-5 | 11-6 | 11-7 |
| | R^{3a} | |
|---|---|---|
| R⁴ | 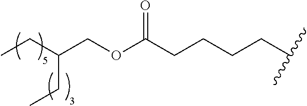 | 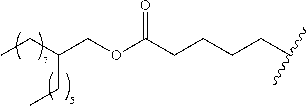 |
| 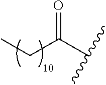 | 1-8 | 1-9 |
| 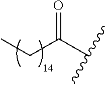 | 2-8 | 2-9 |
| 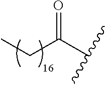 | 3-8 | 3-9 |
| 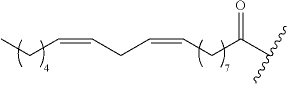 | 4-8 | 4-9 |
| 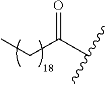 | 5-8 | 5-9 |
| 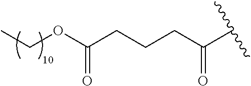 | 6-8 | 6-9 |
| 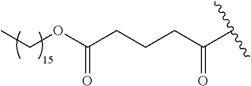 | 7-8 | 7-9 |
| 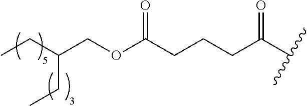 | 8-8 | 8-9 |
| 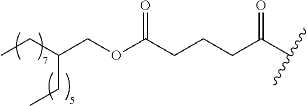 | 9-8 | 9-9 |
| 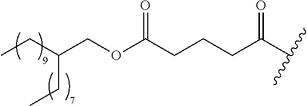 | 10-8 | 10-9 |
| 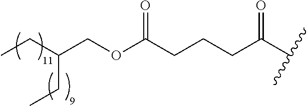 | 11-8 | 11-9 |
| | R^{3a} | |
|---|---|---|
| R⁴ | 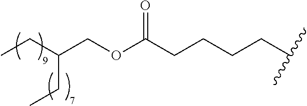 | 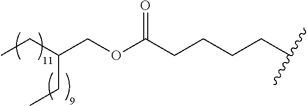 |
| 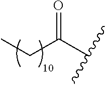 | 1-10 | 1-11 |
| 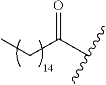 | 2-10 | 2-11 |
| 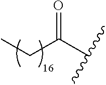 | 3-10 | 3-11 |
| 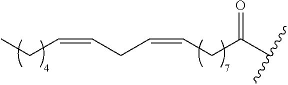 | 4-10 | 4-11 |
| 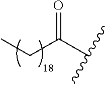 | 5-10 | 5-11 |
| 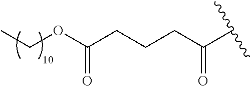 | 6-10 | 6-11 |
| 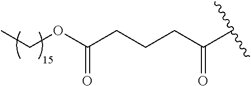 | 7-10 | 7-11 |
| 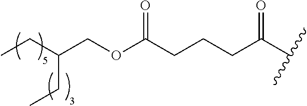 | 8-10 | 8-11 |
| 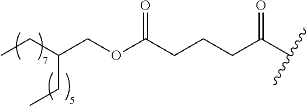 | 9-10 | 9-11 |
| 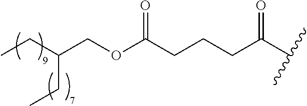 | 10-10 | 10-11 |
| 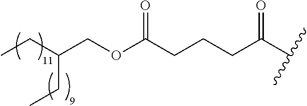 | 11-10 | 11-11. |

79. The compound of claim 1, wherein the compound is of the formula:

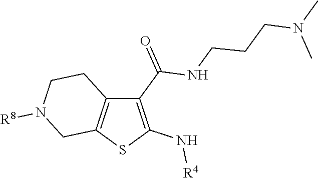

| | R⁴ | | | |
|---|---|---|---|---|
| R⁸ | 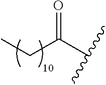 | 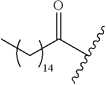 | 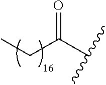 | 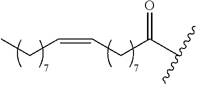 |
| 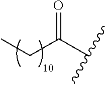 | 12-12 | 12-13 | 12-14 | 12-15 |
| 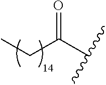 | 13-12 | 13-13 | 13-14 | 13-15 |
| 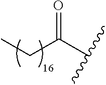 | 14-12 | 14-13 | | 14-15 |
| 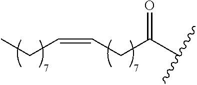 | 15-12 | 15-13 | 15-14 | |
| 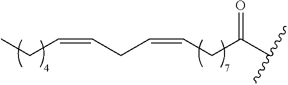 | 16-12 | 16-13 | 16-14 | |
| 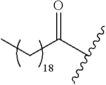 | 17-12 | 17-13 | 17-14 | 17-15 |
| 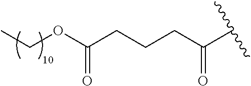 | 18-12 | 18-13 | 18-14 | 18-15 |
| 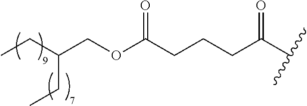 | 19-12 | 19-13 | 19-14 | 19-15 |
| 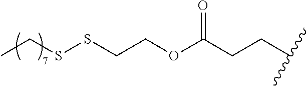 | 20-12 | 20-13 | 20-14 | 20-15 |
| | R⁴ | | |
|---|---|---|---|
| R⁸ | 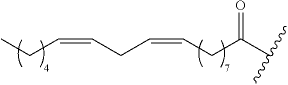 | 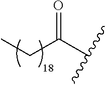 | 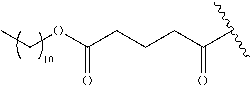 |
| 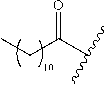 | 12-16 | 12-17 | 12-18 |
| 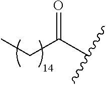 | 13-16 | 13-17 | 13-18 |
| 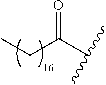 | 14-16 | 14-17 | 14-18 |
| 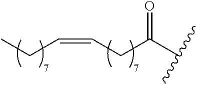 | | 15-17 | 15-18 |
| 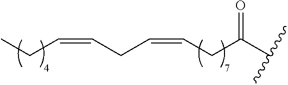 | | 16-17 | 16-18 |
| 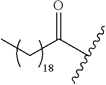 | 17-16 | 17-17 | 17-18 |
| 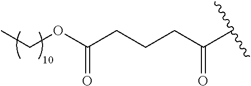 | 18-16 | 18-17 | 18-18 |
| 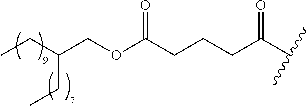 | 19-16 | 19-17 | 19-18 |
| 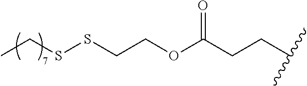 | 20-16 | 20-17 | 20-18 |
| | R⁴ | |
|---|---|---|
| R⁸ | 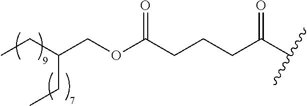 | 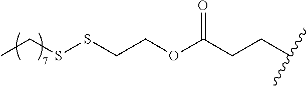 |
| 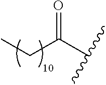 | 12-19 | 12-20 |
| 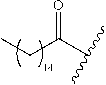 | 13-19 | 13-20 |
| 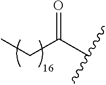 | 14-19 | 14-20 |
| 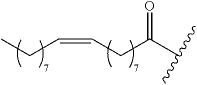 | 15-19 | 15-20 |
| 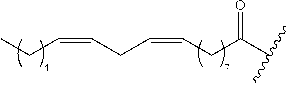 | 16-19 | 16-20 |
| 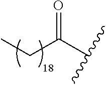 | 17-19 | 17-20 |
| 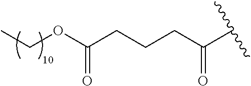 | 18-19 | 18-20 |
| 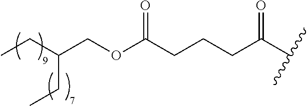 | 19-19 | 19-20 |
| 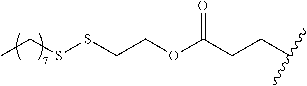 | 20-19 | 20-20. |

80. The compound according to claim 1 in a pharmaceutically acceptable salt form.

81. The compound according to claim 1 not in a pharmaceutically acceptable salt form.

82. A composition comprising a compound according to claim 1.

83. The composition of claim 82, wherein the composition is a nanoparticle.

84. The composition of claim 82, wherein the composition further comprises an agent.

85. The composition of claim 84, wherein the agent comprises a nucleic acid, small molecule drug, protein, polypeptide, antibody, peptide, or a combination thereof.

86. The composition of claim 84, wherein the agent is a nucleic acid.

87. The composition of claim 86, wherein the nucleic acid is not covalently attached to the compound.

88. The composition of claim 86, wherein the nucleic acid is a single stranded DNA, single stranded RNA, double-stranded DNA, RNA-RNA hybrid, DNA-RNA hybrid, shortmer, antagomir, antisense, ribozyme, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), or a combination thereof.

89. The composition of claim 86, wherein the nucleic acid is a plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, viral DNA, circular RNA (circRNA), precursor messenger RNA (pre-mRNA), microRNA (miRNA), guide RNA (gRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, viral satellite RNA, or a combination thereof.

90. The composition of claim 82, wherein the compound is present in the composition in an amount of from 20 mol % to 70 mol %.

91. The composition of claim 82, further comprising a phospholipid, a structural lipid, a polymer-conjugated lipid, or a combination thereof.

92. The composition of claim 91, wherein the phospholipid is 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or a combination thereof.

93. The composition of claim 91, wherein the structural lipid is cholesterol, beta-sitosterol, fucosterol, campesterol, stigmastanol, or a combination thereof.

94. The composition of claim 91, wherein the polymer-conjugated lipid is selected from PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof.

95. The composition of claim 91, wherein the polymer-conjugated lipid comprises a PEG moiety having a number-average molecular weight of from 1000 daltons to 20,000 daltons.

96. The composition of claim 91, wherein the composition is a nanoparticle comprising:
from 0 to 30 mol % of the phospholipid;
from 0 to 60 mol % of the structural lipid; and
from 0 to 10 mol % of the polymer-conjugated lipid;
providing that at least one of the phospholipid, the structural lipid, and the polymer-conjugated lipid is present in the nanoparticle.

97. The composition of claim 91, wherein the polymer-conjugated lipid is (distearoyl-phosphatidyl-ethanolamine)-PEG-C(O)OH or (distearoyl-phosphatidyl-ethanolamine)-PEG-carboxy-NHS.

98. The composition of claim 91 further comprising a sterol, carboxy-PEG, 1,2-distearoyl-sn-glycero-3-phosphocholine or a negatively charged phospholipid, and a PEG-lipid.

99. The composition of claim 98, wherein:
the concentration of the compound is 50%±its 20%;
the concentration of the sterol is 38.5%±its 20%;
the concentration of the carboxy-PEG is between 0 and 0.36%, e.g., between 0.15% and 0.35%, inclusive;
the concentration of 1,2-distearoyl-sn-glycero-3-phosphocholine or the negatively charged phospholipid is 10%±its 20%; and/or
the concentration of the PEG-lipid is 1.2%±its 20%;
provided that the combined concentrations of the compound, sterol, carboxy-PEG, 1,2-distearoyl-sn-glycero-3-phosphocholine or negatively charged phospholipid, and PEG-lipid is 100%.

100. The composition of claim 99, wherein the sterol is cholesterol or beta-sitosterol.

101. The composition of claim 86, wherein a ratio of the nucleic acid to the compound is from 2:1 to 30:1.

102. The composition of claim 86, wherein a wt/wt ratio of total lipid component to nucleic acid is from 5:1 to 60:1.

103. A pharmaceutical composition comprising the composition of claim 82, and a pharmaceutically acceptable excipient.

104. A method for making a nanoparticle according to claim 83, the method comprises:
providing a first solution comprising a compound according to Formula A 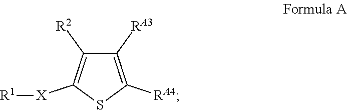
providing a second solution comprising an agent; and
mixing the first and second solutions to form a mixture comprising the nanoparticle;
wherein
X is a bond, —C(O)O—, —OC(O)—, —NH—, —N(R¹)—, —C(O)N(R¹)—, —N(R¹)C(O)—, —C(O)NH—, —NHC(O)—, —C≡C—, —CH═CH—, —O—, —S—, —NHC(O)NH—, —OC(O)O—, —OC(O)NH—, —NHC(O)O—, —O(CH₂)ₘ or —(CH₂)ₘO—;
m is from 0 to 6;
each R¹ is H, halogen, CN, or aliphatic;
R² is H, halogen, aromatic, or aliphatic;
or X is a bond and R¹ and R², together with the atoms to which there are attached, form a 4- to 7-membered nitrogen-containing non-aromatic heterocyclyl;
R^{A3} is halogen, R³, —C(O)N(R⁶)(R⁷), —C(O)R³, —CN, —(CH₂)ᵣN(R⁶)(R⁷), —N(R⁶)(R⁷), —S(O)R^{d}, —S(O)R³, —CH₂SR^{d}, —CH₂R³, —S(O)N(R⁶)(R⁷), or —X¹(CH₂)ᵣN(R^{a})₂;
X¹ is O, S or NH;
r is from 1 to 8;
each R^{a} independently is H, C₁₋₆alkyl, C₃₋₆cycloalkyl, or both R^{a}s together with the nitrogen to which they are attached form a 5- or 6-membered heterocyclyl;
R³ is aliphatic, —O-aliphatic or N(R⁶)(R⁷);
R^{A4} is halogen, boronic acid, —N(R⁴)(R⁵), NHC(O)N(R⁴)(R⁵), —N═CHR^{d}, —NHC(S)N(R⁴)(R⁵), —NHC(O)(CH₂)_{z}—N(R⁴)(R⁵), —NHSO₂R^{d}, —NHS(O)R^{d}, —NHC(O)C(O)NH(CH₂)_{z}—N(R⁴)(R⁵), or —OR⁴, where z is an integer from 1 to 6;
each R^{d} is aliphatic;
R⁴ is aliphatic or —C(O)aliphatic;
R⁵ is H, aliphatic, or —C(O)aliphatic;
R⁶ is H or aliphatic;
R⁷ is aliphatic or C(O)aliphatic;
or R⁶ and R⁷, together with the atom to which there are attached, form a 4- to 7-membered nitrogen-containing heterocyclyl; and
wherein the compound comprises at least one C₆₋₃₀aliphatic moiety.

105. A method for making a nanoparticle according to claim 83, the method comprises:
providing a first solution comprising a compound according to Formula A 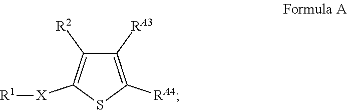
providing a third solution; and
mixing the first and third solutions to form a mixture comprising the nanoparticle;
wherein
X is a bond, —C(O)O—, —OC(O)—, —NH—, —N(R¹)—, —C(O)N(R¹)—, —N(R¹)C(O)—, —C(O)NH—, —NHC(O)—, —C≡C—, —CH═CH—, —O—, —S—, —NHC(O)NH—, —OC(O)O—, —OC(O)NH—, —NHC(O)O—, —O(CH₂)ₘ or —(CH₂)ₘO—;
m is from 0 to 6;
each R¹ is H, halogen, CN, or aliphatic;
R² is H, halogen, aromatic, or aliphatic;
or X is a bond and R¹ and R², together with the atoms to which there are attached, form a 4- to 7-membered nitrogen-containing non-aromatic heterocyclyl;
R^{A3} is halogen, R³, —C(O)N(R⁶)(R⁷), —C(O)R³, —CN, —(CH₂)ᵣN(R⁶)(R⁷), —N(R⁶)(R⁷), —S(O)R^{d}, —S(O)R³, —CH₂SR^{d}, —CH₂R³, —S(O)N(R⁶)(R⁷), or —X¹(CH₂)ᵣN(R^{a})₂;
X¹ is O, S or NH;
r is from 1 to 8;
each R^{a} independently is H, C₁₋₆alkyl, C₃₋₆cycloalkyl, or both R^{a}s together with the nitrogen to which they are attached form a 5- or 6-membered heterocyclyl;
R³ is aliphatic, —O-aliphatic or N(R⁶)(R⁷);
R^{A4} is halogen, boronic acid, —N(R⁴)(R⁵), NHC(O)N(R⁴)(R⁵), —N═CHR^{d}, —NHC(S)N(R⁴)(R⁵), —NHC(O)(CH₂)_{z}—N(R⁴)(R⁵), —NHSO₂R^{d}, —NHS(O)R^{d}, —NHC(O)C(O)NH(CH₂)_{z}—N(R⁴)(R⁵), or —OR⁴, where z is an integer from 1 to 6;
each R^{d} is aliphatic;
R⁴ is aliphatic or —C(O)aliphatic;
R⁵ is H, aliphatic, or —C(O)aliphatic;
R⁶ is H or aliphatic;
R⁷ is aliphatic or C(O)aliphatic;
or R⁶ and R⁷, together with the atom to which there are attached, form a 4- to 7-membered nitrogen-containing heterocyclyl; and
wherein the compound comprises at least one C₆₋₃₀aliphatic moiety.

106. The method of claim 105, wherein the third solution is an aqueous buffer solution.

107. The method of claim 105, further comprising:
providing a fourth solution comprising an agent; and
mixing the mixture comprising the nanoparticle with the fourth solution to form a composition.

108. The method of claim 107, wherein the agent is a nucleic acid.

109. The method of claim 104, further comprising providing a phospholipid, a structural lipid, a polymer-conjugated lipid, or a combination thereof, and wherein mixing the first and second solutions further comprises mixing the phospholipid, the structural lipid, and/or the polymer-conjugated lipid with the first solution, the second solution, or the mixture of the first and second solutions.

110. The method of claim 109, wherein the phospholipid, the structural lipid, the polymer-conjugated lipid, or a combination thereof, are provided in the first solution.

111. A method of using a composition according to claim 82, comprising administering an effective amount of the composition to a subject.

112. The method of claim 111, wherein administering to the subject comprises administration by an intravenous, intramuscular, subretinal, intravitreal or intradermal route.

113. The method of claim 111, wherein administering the nanoparticle comprises administration by an intravenous, intramuscular, subretinal or intravitreal or intradermal route.

114. The method of claim 111, wherein administering to the subject comprises administering to lung, liver and/or spleen tissue.

115. The method of claim 114, wherein administering to the subject comprises administering to lung tissue.

116. A method of delivering a pharmaceutical agent to a subject in need thereof comprising administering to or implanting in the subject in need thereof an effective amount of:
a compound of claim 1; and
a pharmaceutical agent.

117. A method of treating a disease comprising administering to or implanting in a subject in need thereof an effective amount of:
a compound of claim 1; and
a therapeutic agent.

118. A method of preventing a disease comprising administering to or implanting in a subject in need thereof an effective amount of:
a compound of claim 1; and
a prophylactic agent.

119. A method of diagnosing a disease comprising administering to or implanting in a subject in need thereof an effective amount of:
a compound of claim 1; and
a diagnostic agent.

120. The method of claim 117, wherein the disease is an ocular disease.

121. The method of claim 120, wherein the disease is inherited retinal degeneration or macular degeneration.

122. The method of claim 117, wherein the disease is cystic fibrosis.

123. The method of claim 117, wherein the disease is cancer, benign neoplasm, pathologic angiogenesis, inflammatory disease, autoinflammatory disease, autoimmune disease, metabolic disease, neurological disease, genetic disease, hematological disease, painful condition, or psychiatric disease.

124. The method of claim 123, wherein the disease is cancer.

125. Use of a compound of claim 1 in the preparation of a medicament for administration to a subject.

126. The method of claim 111, wherein the subject is a human.

127. A method of making a compound of claim 27, comprising:
when R⁵ is H: reacting a compound of the formula: 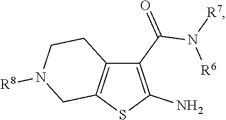
with a compound of the formula: R⁴-(leaving group) or R⁴—OH under suitable conditions to provide the compound of claim 27; or
when R⁵ is aliphatic or —C(O)aliphatic: reacting a compound of the formula: 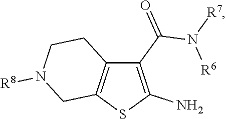
with a compound of the formula: R⁴-(leaving group) or R⁴—OH, and a compound of the formula: R⁵-(leaving group) or R⁵—OH, in the same step or separate steps, under suitable conditions to provide the compound of claim 27.

128. The method of claim 127, further comprising reacting a compound of the formula: 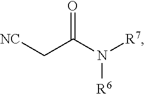
with a compound of the formula: 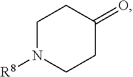
in the presence of S₈ under suitable conditions to provide the compound of the formula:

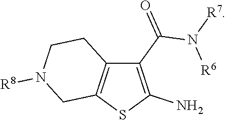

129. The method of claim 127, wherein R⁵ is H.

130. The method of claim 127, wherein R⁴ is —C(O)aliphatic.

131. The method of claim 127, wherein R⁸ is —C(O)aliphatic.

132. The compound of claim 1, wherein the compound comprises only one C₆₋₃₀aliphatic moiety.

133. The compound of claim 1, wherein the compound comprises only two C₆₋₃₀aliphatic moieties.

134. The compound of claim 1, wherein the compound comprises only three C₆₋₃₀aliphatic moieties.

135. The compound of claim 1, wherein the compound comprises only four C₆₋₃₀aliphatic moieties.

136. The compound of claim 1, wherein each aliphatic moiety is unsubstituted.

137. The compound of claim 1, wherein at least one aliphatic moiety is substituted.

138. The compound of claim 1, wherein each aliphatic moiety is linear.

139. The compound of claim 1, wherein at least one aliphatic moiety is branched.

140. A kit comprising:
a compound of claim 1; and
instructions for using the compound or composition.
